(19) United States
(12) Patent Application Publication
(10) Pub. No.: US 2023/0150974 A1
(43) Pub. Date: May 18, 2023

(54) COMPOUNDS AND USES THEREOF

(71) Applicant: Foghorn Therapeutics Inc., Indianapolis, IN (US)

(72) Inventors: Matthew NETHERTON, Cambridge, MA (US); Francois BRUCELLE, Belmont, MA (US)

(21) Appl. No.: 17/907,463

(22) PCT Filed: Apr. 6, 2021
(86) PCT No.: PCT/US2021/026069

§ 371 Date: Sep. 27, 2022

Related U.S. Application Data
(60) Provisional application No. 63/005,829, filed on Apr. 6, 2020.

(51) Int. Cl. C07D 401/14; C07D 417/14; C07D 487/10; C07D 471/10

(52) U.S. Cl. CPC C07D 401/14; C07D 417/14; C07D 487/10; C07D 471/10

(57) ABSTRACT

The present disclosure features compounds useful for the treatment of BAF complex-related disorders.

### BACKGROUND

The invention relates to compounds useful for modulating BRG1- or BRM-associated factors (BAF) complexes. In particular, the invention relates to compounds useful for treatment of disorders associated with BAF complex function.

Chromatin regulation is essential for gene expression, and ATP-dependent chromatin remodeling is a mechanism by which such gene expression occurs. The human Switch/Sucrose Non-Fermentable (SWI/SNF) chromatin remodeling complex, also known as BAF complex, has two SWI2-like ATPases known as BRG1 (Brahma-related gene-1) and BRM (Brahma). The transcription activator BRG1, also known as ATP-dependent chromatin remodeler SMARCA4, is encoded by the SMARCA4 gene on chromosome 19. BRG1 is overexpressed in some cancer tumors and is needed for cancer cell proliferation. BRM, also known as probable global transcription activator SNF2L2 and/or ATP-dependent chromatin remodeler SMARCA2, is encoded by the SMARCA2 gene on chromosome 9 and has been shown to be essential for tumor cell growth in cells characterized by loss of BRG1 function mutations. Deactivation of BRG and/or BRM results in downstream effects in cells, including cell cycle arrest and tumor suppression.

### SUMMARY

The present invention features compounds useful for modulating a BAF complex. In some embodiments, the compounds are useful for the treatment of disorders associated with an alteration in a BAF complex, e.g., a disorder associated with an alteration in one or both of the BRG1 and BRM proteins. The compounds of the invention, alone or in combination with other pharmaceutically active agents, can be used for treating such disorders.

In an aspect, the invention features a compound having the structure of Formula I:

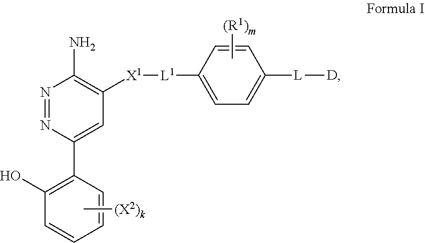

where

X¹ is O or NR²;

each X² is independently a halogen;

k is 0, 1, 2, 3, or 4;

m is 0, 1, 2, 3, or 4;

R¹ is halo or optionally substituted C₁-C₆ alkyl;

R² is H or optionally substituted C₁-C₆ alkyl;

L¹ is optionally substituted C₁-C₆ alkylene;

L is a linker including the structure of

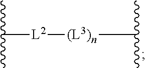

n is 0, 1, 2, or 3;

L² is optionally substituted C₁-C₆ alkylene, optionally substituted C₁-C₂₀ heteroalkylene, or optionally substituted C₂-C₉ heterocyclylene;

each L³ is, independently, —O—, optionally substituted C₁-C₂₀ heteroalkylene, optionally substituted C₃-C₁₀ carbocyclylene, optionally substituted C₃-C₁₀ carbocyclylene-C₁-C₂₀ alkylene, optionally substituted C₂-C₉ heterocyclylene, or optionally substituted C₂-C₉ heterocyclylene-C₁-C₂₀ alkylene; and

D is a degradation moiety,

or a pharmaceutically acceptable salt thereof.

In some embodiments, m is 0. In some embodiments, m is 1 or 2.

In some embodiments, X¹ is O. In some embodiments, X¹ is NR².

In some embodiments, R² is optionally substituted C₁-C₆ alkyl. In some embodiments, R² is methyl or ethyl. In some embodiments, R² is methyl.

In some embodiments, L¹ is

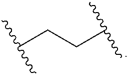 [SMILES: *CC*]

In some embodiments, L² is optionally substituted C₁-C₆ alkylene or optionally substituted C₁-C₂₀ heteroalkylene. In some embodiments, L² is optionally substituted C₂-C₉ heterocyclylene.

In some embodiments, L² is optionally substituted C₁-C₆ alkylene. In some embodiments, L² is optionally substituted C₁-C₂₀ heteroalkylene.

In some embodiments, L² is

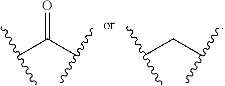

In some embodiments, L² is

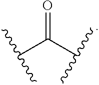 [SMILES: *C(*)=O]

In some embodiments, L² is

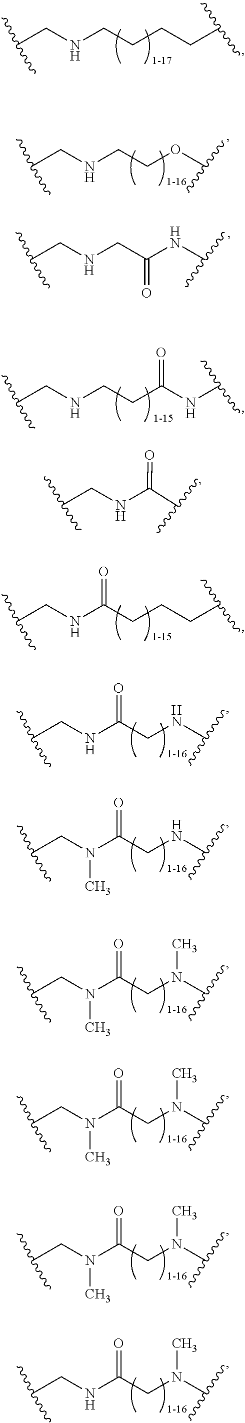

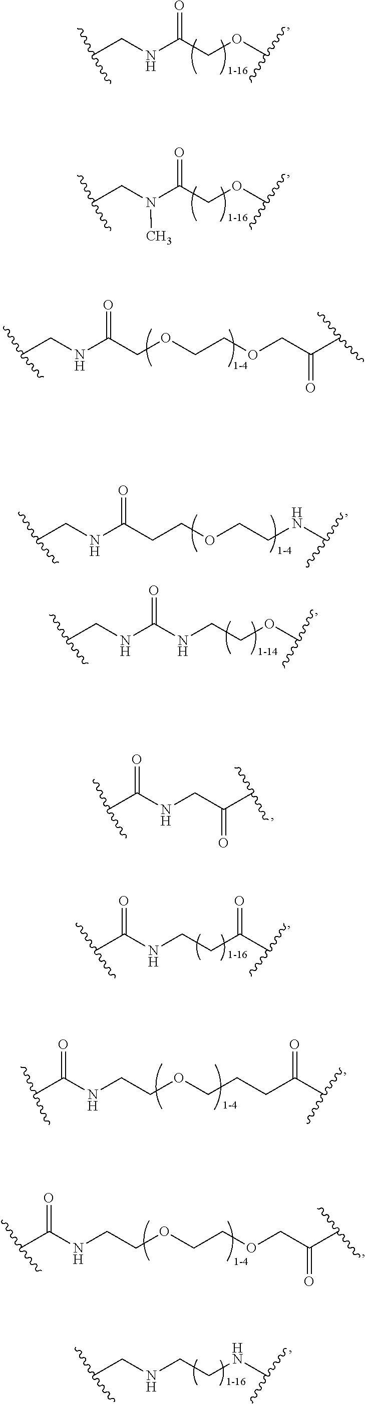

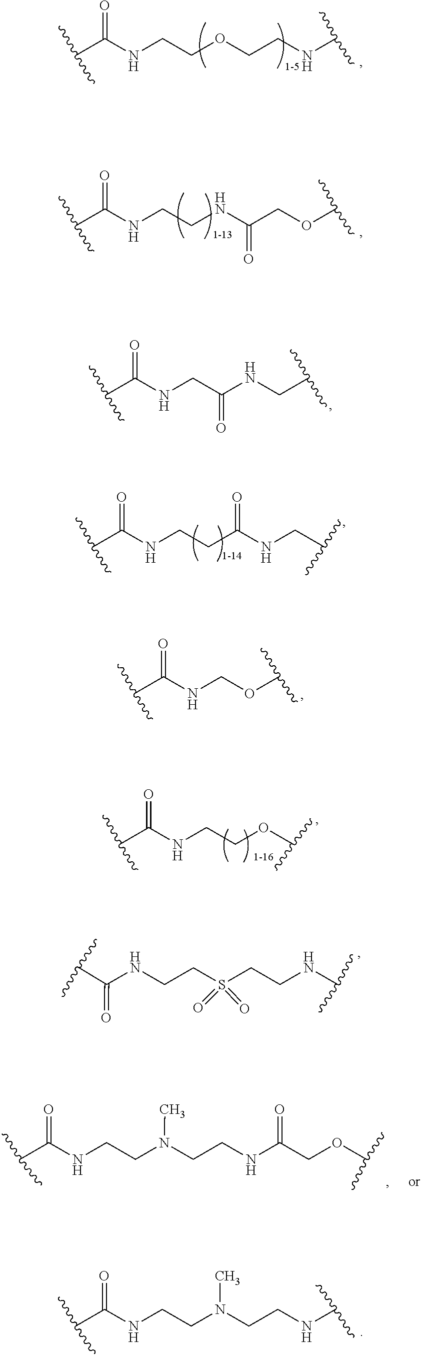

In some embodiments, n is 1. In some embodiments, n is 2. In some embodiments, n is 3.

In some embodiments, each L³ is, independently, optionally substituted C₁-C₂₀ heteroalkylene, optionally substituted C₃-C₁₀ carbocyclylene, optionally substituted C₃-C₁₀ carbocyclylene-C₁-C₆ alkylene, optionally substituted C₂-C₉ heterocyclylene, or optionally substituted C₂-C₉ heterocyclylene-C₁-C₆ alkylene. In some embodiments, each L³ is, independently, optionally substituted C₃-C₁₀ carbocyclylene-C₁-C₆ alkylene, optionally substituted C₂-C₉ heterocyclylene, or optionally substituted C₂-C₉ heterocyclylene-C₁-C₆ alkylene.

In some embodiments, each L³ is, independently,

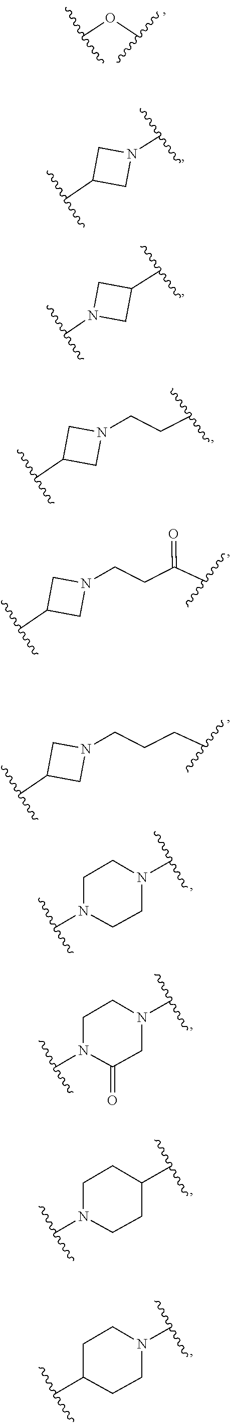

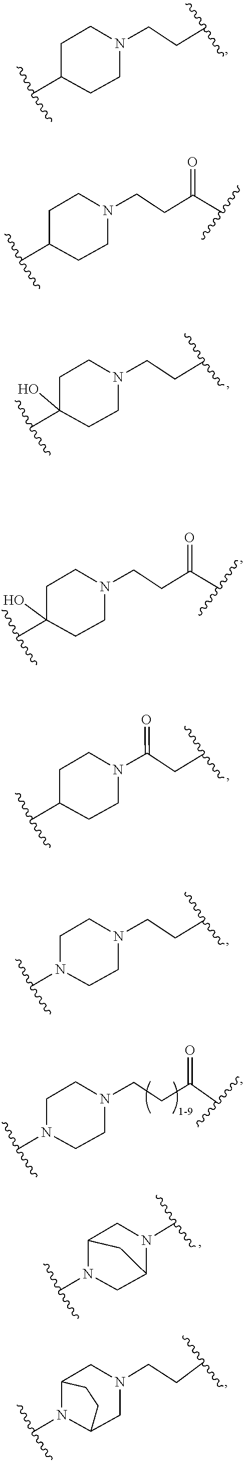

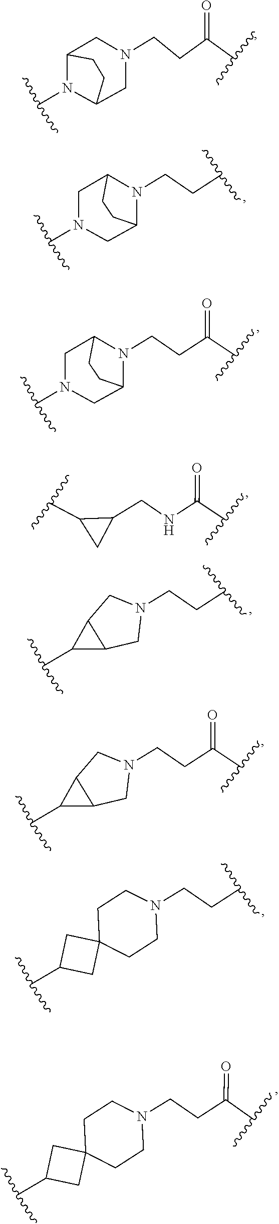

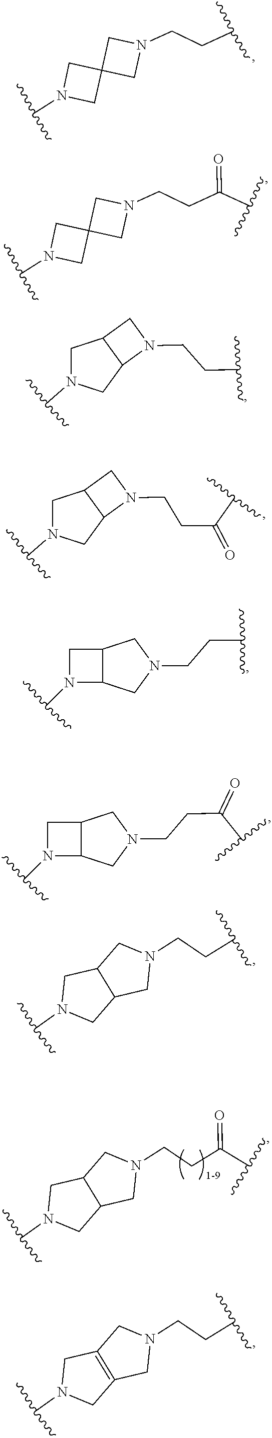

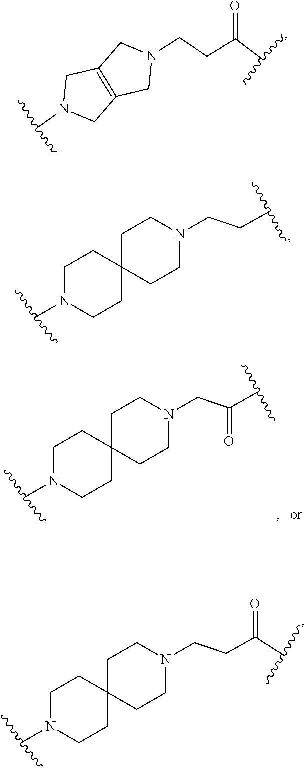 [SMILES: *C(=O)CCN1CC2=C(CN(*)C2)C1 ; *CCN1CCC2(CCN(*)CC2)CC1 ; *C(=O)CN1CCC2(CCN(*)CC2)CC1 ; *C(=O)CCN1CCC2(CCN(*)CC2)CC1]

In some embodiments, each L³ is, independently,

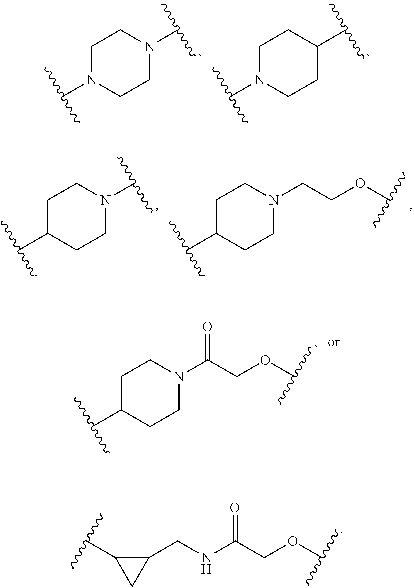 [SMILES: *N1CCN(*)CC1 ; *C1CCN(*)CC1 ; *C1CCN(*)CC1 ; *OCCN1CCC(*)CC1 ; *OCC(=O)N1CCC(*)CC1 ; *OCC(=O)NCC1CC1*]

In some embodiments, n is 0.

In some embodiments, k is 0, 1, or 2. In some embodiments, each X² is independently fluorine or chlorine.

In some embodiments, the compound is of Formula Ib:

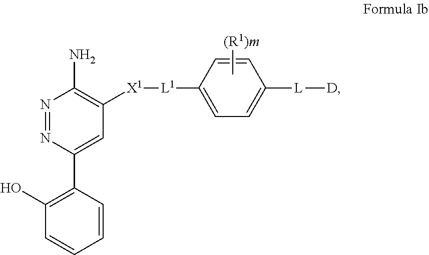

or a pharmaceutically acceptable salt thereof.

In an aspect, the invention features a compound having the structure of Formula II:

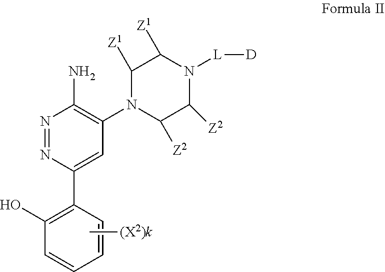

where

one Z¹ and one Z² combine to form an optionally substituted C₁-C₄ alkylene, and the remaining Z¹ and Z² are each hydrogen;

each X² is independently a halogen;

k is 0, 1, 2, 3, or 4;

L is a linker having the structure of

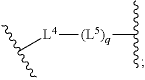

q is 0, 1, 2, 3, or 4;

L⁴ is optionally substituted C₁-C₆ alkylene, optionally substituted C₁-C₂₀ heteroalkylene, or optionally substituted C₂-C₉ heteroarylene;

each L⁵ is independently —O—, optionally substituted C₁-C₆ alkylene, optionally substituted C₁-C₂₀ heteroalkylene, optionally substituted C₃-C₁₀ carbocyclylene, optionally substituted C₃-C₁₀ carbocyclylene-C₁-C₆ alkylene, optionally substituted C₂-C₉ heterocyclylene, or C₂-C₉ heterocyclylene-C₁-C₂₀ alkylene; and

D is a degradation moiety,

or a pharmaceutically acceptable salt thereof.

In some embodiments, the compound is of the compound is of Formula IIa:

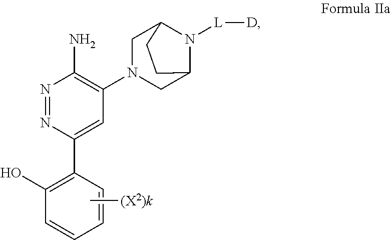

or a pharmaceutically acceptable salt thereof.

In some embodiments, the compound is of Formula IIb:

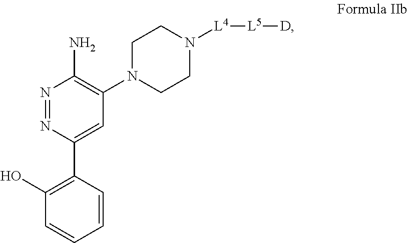

where

L⁴ is optionally substituted C₁-C₆ alkylene or optionally substituted C₁-C₂₀ heteroalkylene;

L⁵ is absent, optionally substituted C₃-C₁₀ carbocyclylene-C₁-C₆ alkylene, or optionally substituted C₂-C₉ heterocyclylene-C₁-C₆ alkylene; and

D is a degradation moiety,

or a pharmaceutically acceptable salt thereof.

In some embodiments, q is 1. In some embodiments, q is 2. In some embodiments, q is 3. In some embodiments, q is 4.

In some embodiments, L is

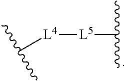

In some embodiments, L⁴ is optionally substituted C₁-C₆ alkylene. In some embodiments, L⁴ is optionally substituted C₁-C₂₀ heteroalkylene.

In some embodiments, L⁴ is

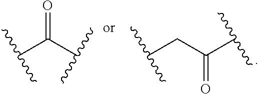

In some embodiments, L⁴ is

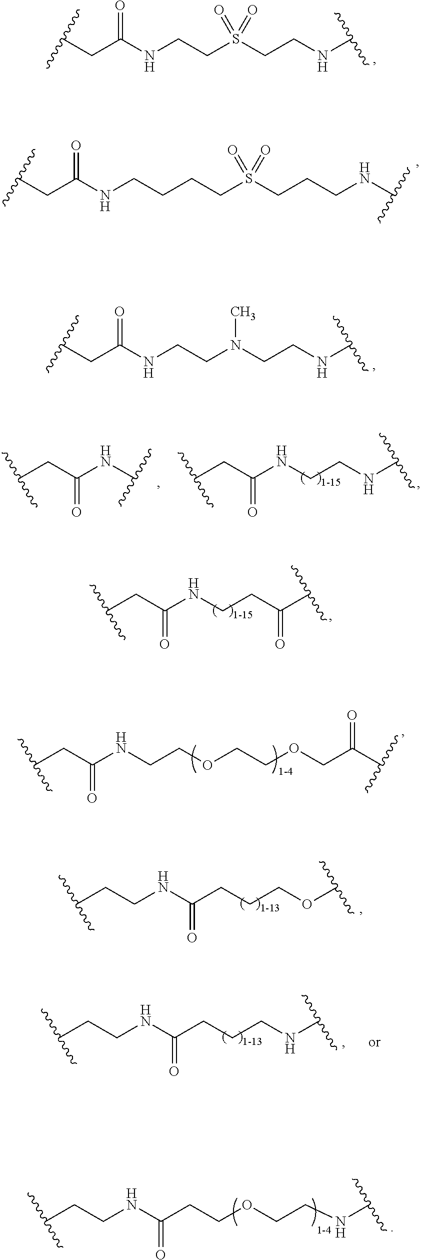

In some embodiments, L⁵ is absent. In some embodiments, each L⁵ is independently —O—, optionally substituted C₁-C₆ alkylene, optionally substituted C₁-C₂₀ heteroalkylene, optionally substituted C₃-C₁₀ carbocyclylene, optionally substituted C₃-C₁₀ carbocyclylene-C₁-C₆ alkylene or optionally substituted C₂-C₉ heterocyclylene-C₁-C₆ alkylene. In some embodiments, each L⁵ is independently optionally substituted C₃-C₁₀ carbocyclylene-C₁-C₆ alkylene or optionally substituted C₂-C₉ heterocyclylene-C₁-C₆ alkylene.

In some embodiments, (L⁵)_{q} is

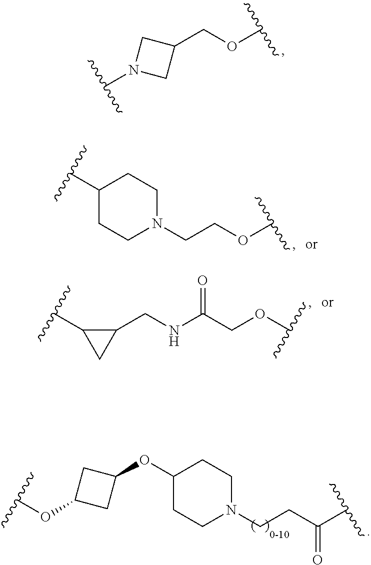

In some embodiments, (L⁵)_{q} is

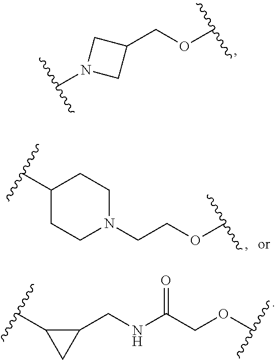 [SMILES: *OCC1CN(*)C1 ; *OCCN1CCC(*)CC1 ; *OCC(=O)NCC1CC1*]

In some embodiments, the compound is of Formula IIb:

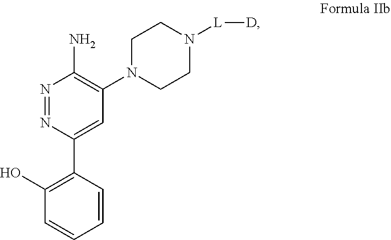

or a pharmaceutically acceptable salt thereof.

In some embodiments of any of the foregoing compounds, the degradation moiety is a ubiquitin ligase binding moiety.

In some embodiments, the ubiquitin ligase binding moiety includes a Cereblon ligand, an IAP (Inhibitors of Apoptosis) ligand, a mouse double minute 2 homolog (MDM2), or a von Hippel-Lindau ligand, or derivatives or analogs thereof.

In some embodiments, the degradation moiety includes the structure of Formula Y:

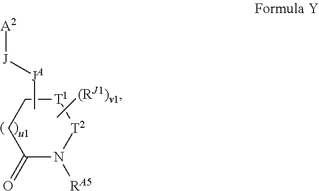

where

A² is a bond between the degradation moiety and the linker;

v1 is 0, 1, 2, 3, 4, or 5;

u1 is 1, 2, or 3;

T¹ is a bond or

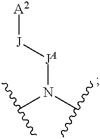

T² is

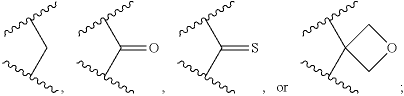

R^{5A} is H, optionally substituted C₁-C₆ alkyl, or optionally substituted C₁-C₆ heteroalkyl; each R^{J1} is, independently, halogen, optionally substituted C₁-C₆ alkyl, or optionally substituted C₁-C₆ heteroalkyl;

J^{A} is absent, O, optionally substituted amino, optionally substituted C₁-C₆ alkyl, or optionally substituted C₁-C₆ heteroalkyl; and

J is absent, optionally substituted C₃-C₁₀ carbocyclylene, optionally substituted C₆-C₁₀ arylene, optionally substituted C₂-C₉ heterocyclylene, or optionally substituted C₂-C₉ heteroarylene, or a pharmaceutically acceptable salt thereof.

In some embodiments, T² is

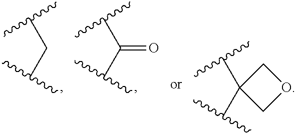

In some embodiments, T² is

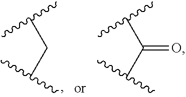

In some embodiments, T² is

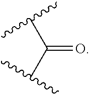 [SMILES: *C(*)=O]

In some embodiments, T² is

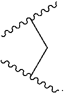 [SMILES: *C*]

In some embodiments, the degradation moiety includes the structure of Formula Y1:

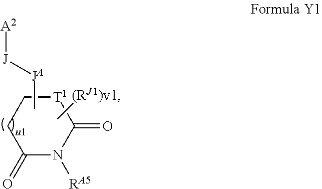

or a pharmaceutically acceptable salt thereof.

In some embodiments, T¹ is a bond. In some embodiments, T¹ is

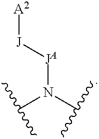

In some embodiments, the degradation moiety includes the structure of Formula Y2:

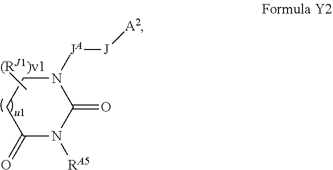

or a pharmaceutically acceptable salt thereof.

In some embodiments, the degradation moiety includes the structure of Formula Z:

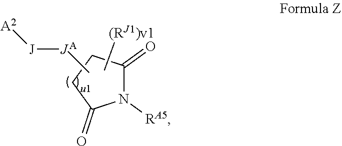

or a pharmaceutically acceptable salt thereof.

In some embodiments, u1 is 1. In some embodiments, u1 is 2. In some embodiments u1 is 3.

In some embodiments, the degradation moiety includes the structure of Formula AA0:

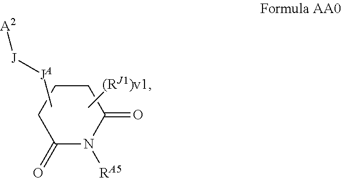

or a pharmaceutically acceptable salt thereof.

In some embodiments, the degradation moiety includes the structure of Formula AB:

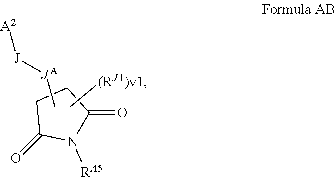

or a pharmaceutically acceptable salt thereof.

In some embodiments, the degradation moiety includes the structure of Formula AC:

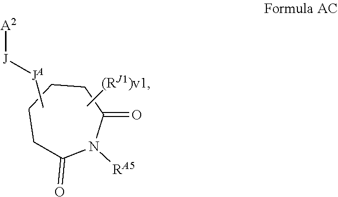

or a pharmaceutically acceptable salt thereof.

In some embodiments, J^{A} is absent. In some embodiments, J^{A} is optionally substituted C₁-C₆ alkyl. In some embodiments, J^{A} is optionally substituted C₁-C₆ heteroalkyl. In some embodiments, J^{A} is O or optionally substituted amino.

In some embodiments, J^{A} is

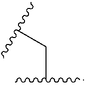 [SMILES: *C*]

In some embodiments, the degradation moiety includes the structure of Formula AA0:

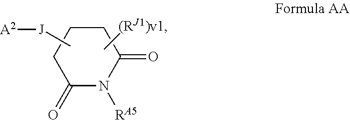

or a pharmaceutically acceptable salt thereof.

In some embodiments, v1 is 0, 1, 2, or 3. In some embodiments, v1 is 0. In some embodiments, v1 is 1. In some embodiments, v1 is 2. In some embodiments, v1 is 3.

In some embodiments, the degradation moiety includes the structure of Formula AA1:

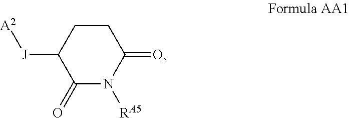

or a pharmaceutically acceptable salt thereof.

In some embodiments, the degradation moiety includes the structure of Formula AB1:

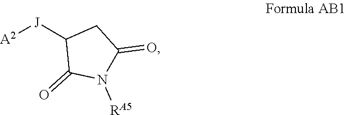

or a pharmaceutically acceptable salt thereof.

In some embodiments, the degradation moiety includes the structure of Formula AC1:

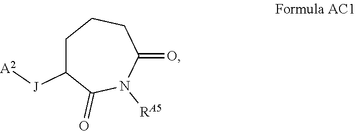

or a pharmaceutically acceptable salt thereof.

In some embodiments, J is absent. In some embodiments, J is optionally substituted C₃-C₁₀ carbocyclylene or optionally substituted C₆-C₁₀ arylene. In some embodiments, J is optionally substituted C₂-C₉ heterocyclylene or optionally substituted C₂-C₉ heteroarylene.

In some embodiments, J is optionally substituted heterocyclylene. In some embodiments, J is optionally substituted C₆-C₁₀ arylene.

In some embodiments, J is

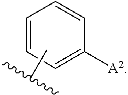

In some embodiments, the degradation moiety includes the structure of Formula AA2:

or a pharmaceutically acceptable salt thereof.

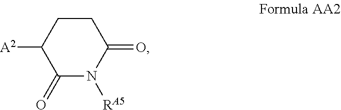

In some embodiments, the degradation moiety includes the structure of Formula AA3:

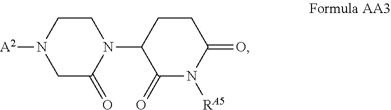

or a pharmaceutically acceptable salt thereof.

In some embodiments, the degradation moiety includes the structure of Formula AA4:

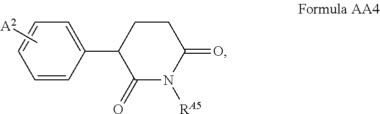

or a pharmaceutically acceptable salt thereof.

In some embodiments, R^{A5} is H or optionally substituted C₁-C₆ alkyl. In some embodiments, R^{A5} is optionally substituted C₁-C₆ heteroalkyl.

In some embodiments, R^{A5} is H or methyl. In some embodiments, R^{A5} is H. In some embodiments, R^{A5} is methyl. In some embodiments, R^{A5} is

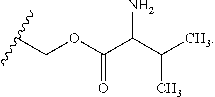 [SMILES: *COC(=O)C(N)C(C)C]

In some embodiments, the degradation moiety includes the structure of Formula A:

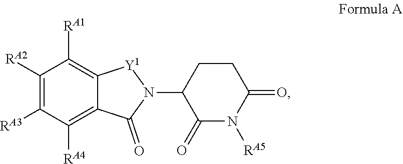

where

Y¹ is

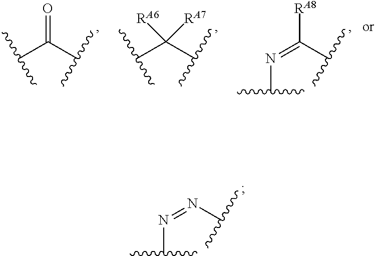

R^{A5} is H, optionally substituted C₁-C₆ alkyl, or optionally substituted C₁-C₆ heteroalkyl;

R^{A6} is H or optionally substituted C₁-C₆ alkyl; and R^{A7} is H or optionally substituted C₁-C₆ alkyl; or R^{A6} and R^{A7}, together with the carbon atom to which each is bound, combine to form optionally substituted C₃-C₆ carbocyclyl or optionally substituted C₂-C₅ heterocyclyl; or R^{A6} and R^{A7}, together with the carbon atom to which each is bound, combine to form optionally substituted C₃-C₆ carbocyclyl or optionally substituted C₂-C₅ heterocyclyl;

R^{A8} is H, optionally substituted C₁-C₆ alkyl, or optionally substituted C₁-C₆ heteroalkyl;

each of R^{A1}, R^{A2}, R^{A3}, and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form

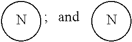

is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A², where one of R^{A1}, R^{A2}, R^{A3}, and R^{A4} is A², or

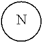

is substituted with A², or a pharmaceutically acceptable salt thereof.

In some embodiments, each of R^{A1}, R^{A2}, R^{A3}, and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form

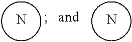

is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A², where one of R^{A1}, R^{A2}, R^{A3}, and R^{A4} is A², or

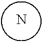

is substituted with A², or a pharmaceutically acceptable salt thereof.

In some embodiments, each of R^{A1}, R^{A2}, R^{A3}, and R^{A4} is, H, A², halogen, optionally substituted C₁-C₆ alkyl, optionally substituted C₁-C₆ heteroalkyl, optionally substituted —O—C₃-C₆ carbocyclyl, hydroxyl, optionally substituted amino; or R^{A1} and R^{A2}, R^{A2} and R^{A3}, or R^{A3} and R^{A4}, together with the carbon atoms to which each is attached, combine to form

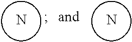

and is optionally substituted C₂-C₉ heterocyclyl, which is optionally substituted with A², where one of R^{A1}, R^{A2}, R^{A3}, and R^{A4} is A², or

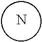

is substituted with A².

In some embodiments, each of R^{A1}, R^{A2}, R^{A3}, and R^{A4} is, independently, H, A², F,

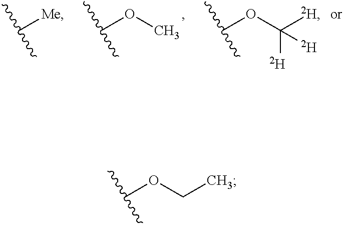

or R^{A1} and R^{A2}, R^{A2} and R^{A3}, or R^{A3} and R^{A4}, together with the carbon atoms to which each is attached, combine to form

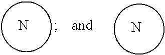

is optionally substituted C₂-C₉ heterocyclyl, which is optionally substituted with A², where one of R^{A1}, R^{A2}, R^{A3}, and R^{A4} is A², or

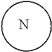

is substituted with A².

In some embodiments, R^{A1} is A². In some embodiments, R^{A2} is A². In some embodiments, R^{A3} is A². In some embodiments, R^{A4} is A². In some embodiments, R^{A5} is A².

In some embodiments, each of R^{A1}, R^{A2}, R^{A3}, and R^{A4} is, independently, H or A².

In some embodiments, R^{A1} is A² and each of R^{A2}, R^{A3}, and R^{A4} is H. In some embodiments, R^{A2} is A² and each of R^{A1}, R^{A3}, and R^{A4} is H. In some embodiments, R^{A3} is A² and each of R^{A1}, R^{A2}, and R^{A4} is H. In some embodiments, R^{A4} is A² and each of R^{A1}, R^{A2}, and R^{A3} is H.

In some embodiments, R^{A5} is H or optionally substituted C₁-C₆ alkyl. In some embodiments, R^{A5} is H or

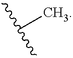

In some embodiments, R^{A5} is H. In some embodiments, R^{A5} is

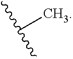

In some embodiments, R^{A8} is H or optionally substituted C₁-C₆ alkyl. In some embodiments, R^{A8} is H or

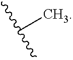

In some embodiments, R^{A8} is H. In some embodiments, R^{A8} is

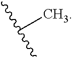

In some embodiments, Y¹ is

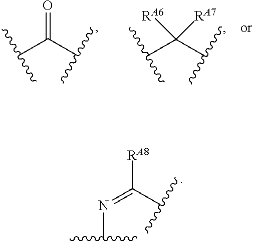

In some embodiments, Y¹ is

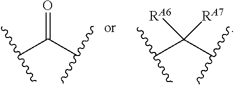

In some embodiments, Y¹ is

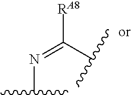

In some embodiments, Y¹ is

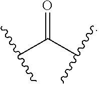 [SMILES: *C(*)=O]

In some embodiments, Y¹ is

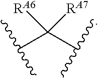

In some embodiments, Y¹ is

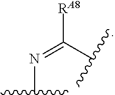

In some embodiments, Y¹ is

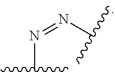 [SMILES: */N=N\*]

In some embodiments, each of R^{A6} and R^{A7} is, independently, H, F,

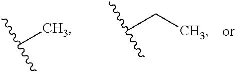

or R^{A6} and R^{A7}, together with the carbon atom to which each is bound, combine to form

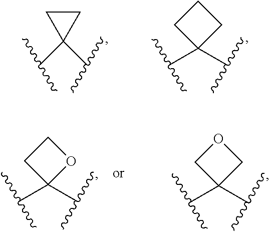 [SMILES: *C1(*)CC1 ; *C1(*)CCC1 ; *C1(*)CCO1 ; *C1(*)COC1]

In some embodiments, R^{A6} is H and R^{A7} is H.

In some embodiments, Y¹ is

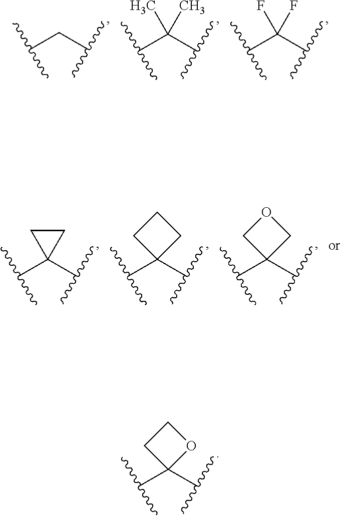

In some embodiments, Y¹ is

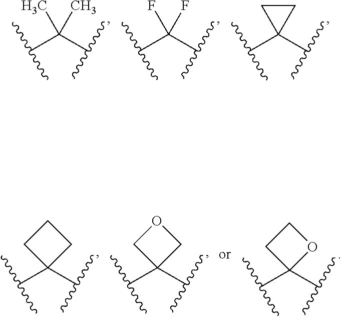

In some embodiments, Y¹ is

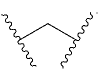 [SMILES: *C*]

In some embodiments, the degradation moiety includes the structure of Formula A1:

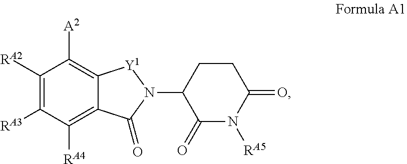

or a pharmaceutically acceptable salt thereof.

In some embodiments, the degradation moiety includes the structure of Formula A2:

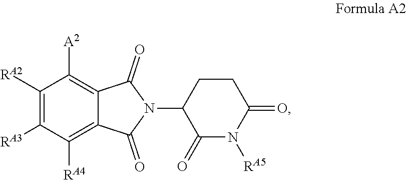

or a pharmaceutically acceptable salt thereof.

In some embodiments, the degradation moiety includes the structure of Formula A3:

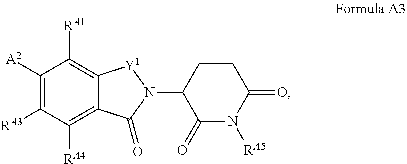

or a pharmaceutically acceptable salt thereof.

In some embodiments, the degradation moiety includes the structure of Formula A4:

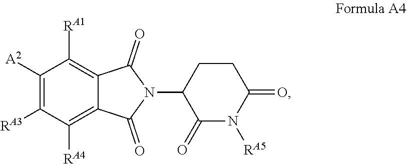

or a pharmaceutically acceptable salt thereof.

In some embodiments, the degradation moiety includes the structure of Formula A5:

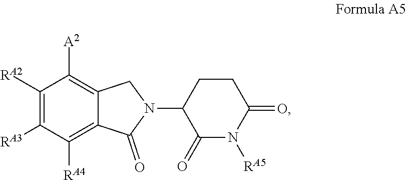

or a pharmaceutically acceptable salt thereof.

In some embodiments, the degradation moiety includes the structure of Formula A6:

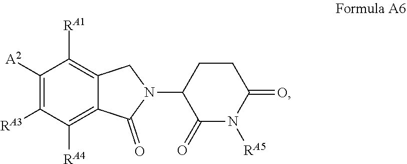

or a pharmaceutically acceptable salt thereof.

In some embodiments, the degradation moiety includes the structure of Formula A7:

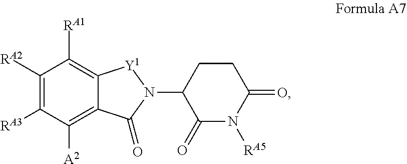

or a pharmaceutically acceptable salt thereof.

In some embodiments, the degradation moiety includes the structure of Formula A8:

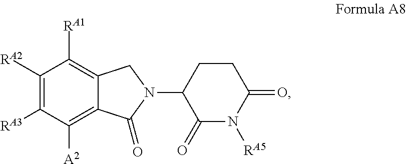

or a pharmaceutically acceptable salt thereof.

In some embodiments, the degradation moiety includes the structure of Formula A9:

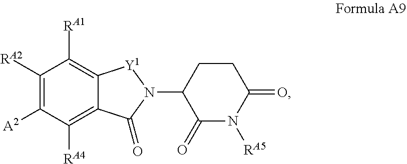

or a pharmaceutically acceptable salt thereof.

In some embodiments, the degradation moiety includes the structure of Formula A10:

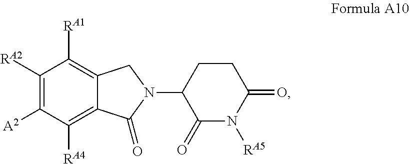

or a pharmaceutically acceptable salt thereof.

In some embodiments, where the degradation moiety includes the structure of

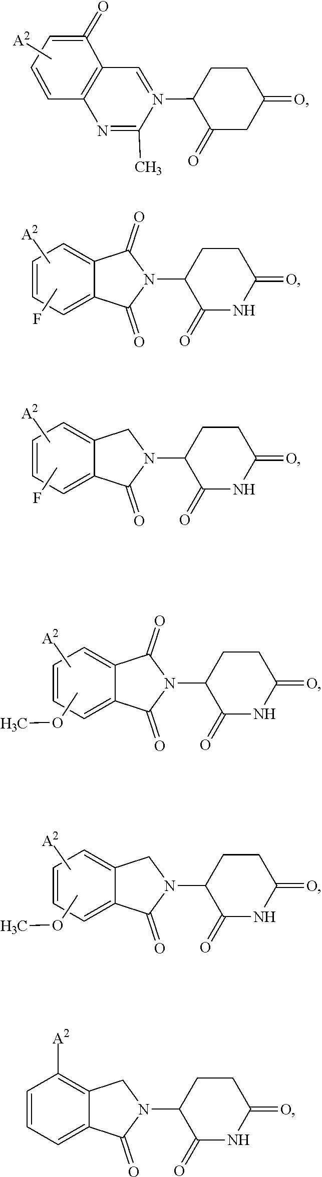

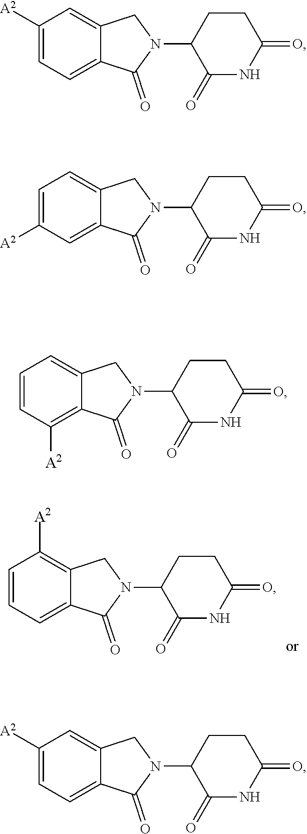

or derivative or analog thereof.

In some embodiments, the degradation moiety includes the structure of

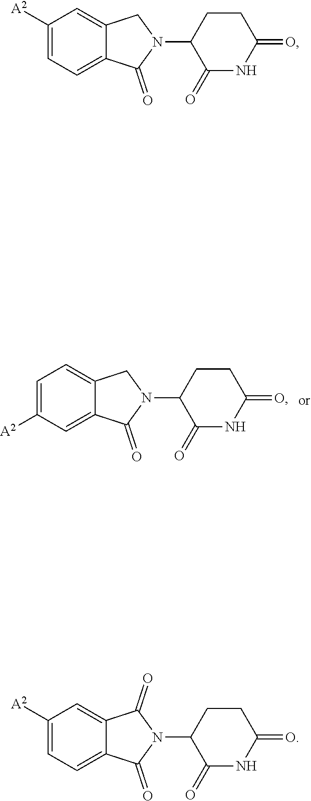

In some embodiments, the degradation moiety includes the structure of

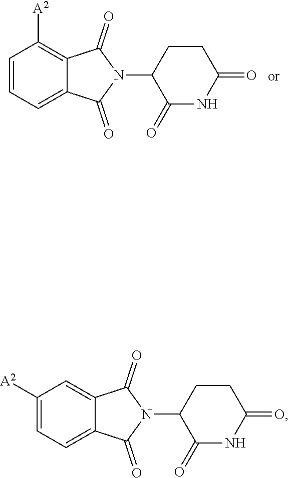

or derivative or analog thereof.

In some embodiments, where the degradation moiety includes the structure of

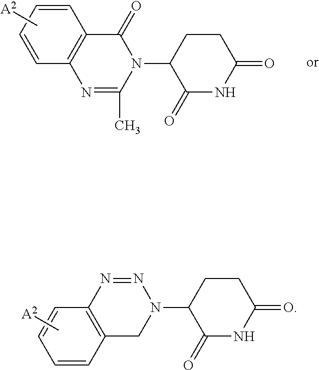

In some embodiments, the degradation moiety includes the structure of

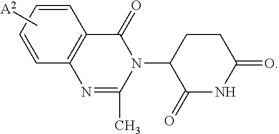

In some embodiments, the degradation moiety includes the structure of

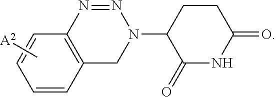

In some embodiments,

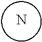

is

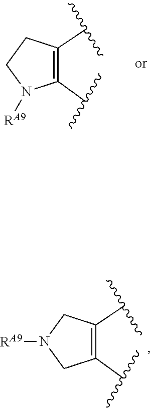

where R^{A9} is H, A², optionally substituted C₁-C₆ alkyl, or optionally substituted C₁-C₆ heteroalkyl.

In some embodiments, the degradation moiety includes the structure of

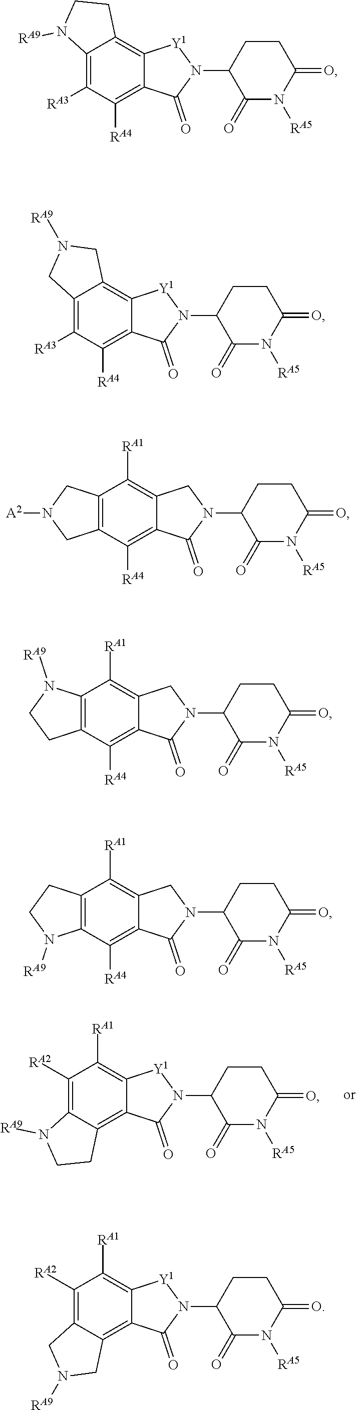

In some embodiments, R^{A9} is H, A², or optionally substituted C₁-C₆ alkyl. In some embodiments, R^{A9} is H, A², or methyl. In some embodiments, R^{9A} is H. In some embodiments, R^{9A} is methyl. In some embodiments, R^{A9} is A².

In some embodiments, the degradation moiety includes the structure of

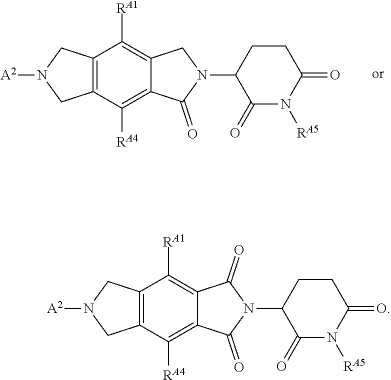

In some embodiments, the degradation moiety includes the structure of Formula B:

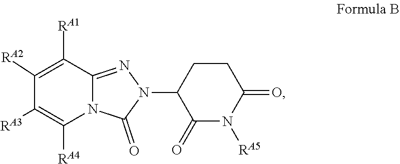

where

R^{A5} is H, optionally substituted C₁-C₆ alkyl, or optionally substituted C₁-C₆ heteroalkyl;

each of R^{A1}, R^{A2}, R^{A3}, and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form

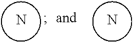

is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A², where one of R^{A1}, R^{A2}, R^{A3}, and R^{A4} is A², or

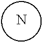

is substituted with A², or a pharmaceutically acceptable salt thereof.

In some embodiments, each of R^{A1}, R^{A2}, R^{A3}, and R^{A4} is, H, A², halogen, optionally substituted C₁-C₆ alkyl, optionally substituted C₁-C₆ heteroalkyl, optionally substituted —O—C₃-C₆ carbocyclyl, hydroxyl, optionally substituted amino; or R^{A1} and R^{A2}, R^{A2}, and R^{A3}, or R^{A3} and R^{A4}, together with the carbon atoms to which each is attached, combine to form

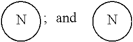

is optionally substituted C₂-C₉ heterocyclyl, which is optionally substituted with A², where one of R^{A1}, R^{A2}, R^{A3}, and R^{A4} is A², or

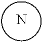

is substituted with A².

In some embodiments, each of R^{A1}, R^{A2}, R^{A3}, and R^{A4} is, independently, H, A², F,

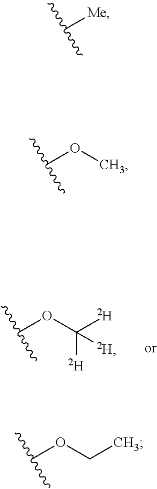

or R^{A1} and R^{A2}, R^{A2} and R^{A3}, or R^{A3} and R^{A4}, together with the carbon atoms to which each is attached, combine to form

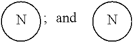

is optionally substituted C₂-C₉ heterocyclyl, which is optionally substituted with A², where one of R^{A1}, R^{A2}, R^{A3}, and R^{A4} is A², or

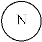

is substituted with A².

In some embodiments, R^{A1} is A². In some embodiments, R^{A2} is A². In some embodiments, R^{A3} is A². In some embodiments, R^{A4} is A². In some embodiments, R^{A5} is A².

In some embodiments, R^{A5} is H or optionally substituted C₁-C₆ alkyl.

In some embodiments, R^{A5} is H or

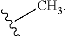

In some embodiments, R^{A5} is H. In some embodiments, R^{A5} is

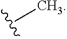

In some embodiments, the degradation moiety includes the structure of Formula B1:

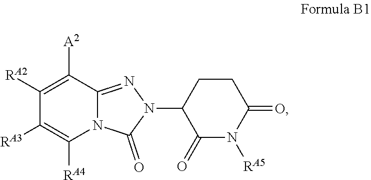

or a pharmaceutically acceptable salt thereof.

In some embodiments, the degradation moiety includes the structure of Formula B2:

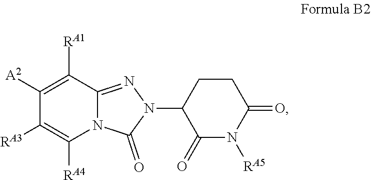

or a pharmaceutically acceptable salt thereof.

In some embodiments, the degradation moiety includes the structure of Formula B3:

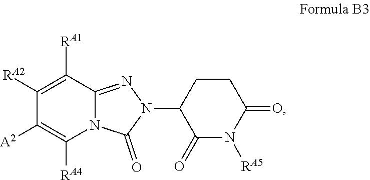

or a pharmaceutically acceptable salt thereof.

In some embodiments, the degradation moiety includes the structure of Formula B4:

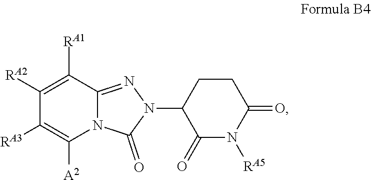

or a pharmaceutically acceptable salt thereof.

In some embodiments, the degradation moiety includes the structure of

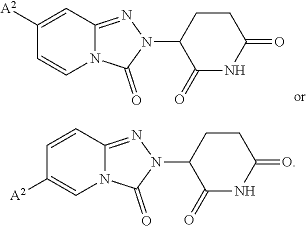

In some embodiments, the degradation moiety includes the structure of

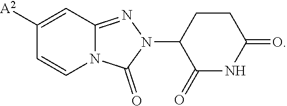

In some embodiments, the degradation moiety includes the structure of

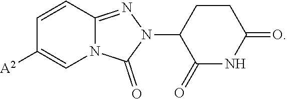

In some embodiments, the ubiquitin ligase binding moiety includes a von Hippel-Lindau ligand.

In some embodiments, the von Hippel-Lindau ligand includes the structure of

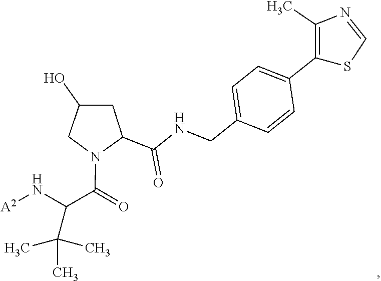

where A² is a bond between the degradation moiety and the linker, or derivative or analog thereof.

In some embodiments, the degradation moiety includes the structure of Formula C:

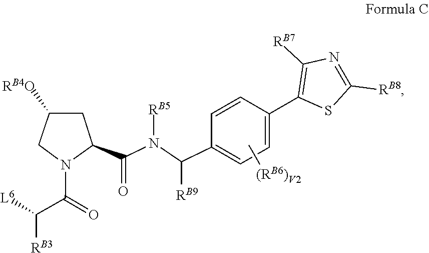

where

L⁶ is —N(R^{B1})(R^{B2}),

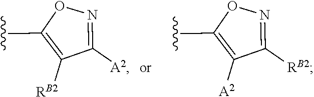

R^{B1} is H, A², optionally substituted C₁-C₆ alkyl, or optionally substituted C₁-C₆ heteroalkyl;

R^{B2} is H, optionally substituted C₁-C₆ alkyl, or optionally substituted C₁-C₆ heteroalkyl;

R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;

R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;

R^{B5} is H, optionally substituted C₁-C₆ alkyl, or optionally substituted C₁-C₆ heteroalkyl;

v2 is 0, 1, 2, 3, or 4;

each R^{B6} is, independently, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; each of R^{B7} and R^{B8} is, independently, H, halogen, optionally substituted C₁-C₆ alkyl, or optionally substituted C₆-C₁₀ aryl;

R^{B9} is H or optionally substituted C₁-C₆ alkyl; and

A² is a bond between the degradation moiety and the linker;

where one and only one of R^{B1}, R^{B3}, and R^{B6} is A².

In some embodiments, the degradation moiety has the structure of Formula C1:

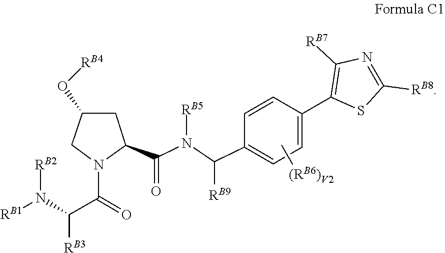

In some embodiments, the degradation moiety has the structure of Formula C2:

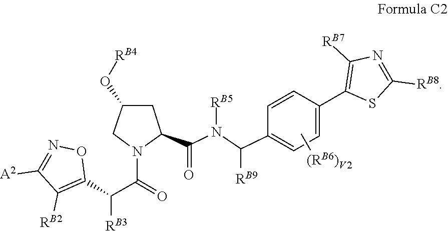

In some embodiments, R^{B9} is optionally substituted C₁-C₆ alkyl. In some embodiments, R^{B9} is methyl. In some embodiments, R^{B9} is bonded to (S)-stereogenic center. In some embodiments, R^{B9} is hydrogen.

In some embodiments, the degradation moiety has the following structure:

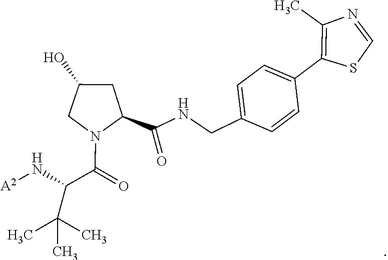

In some embodiments, the degradation moiety has the following structure:

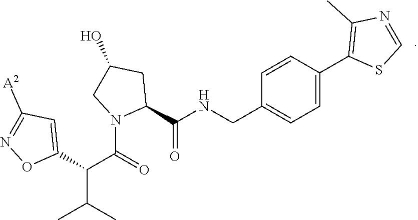

In some embodiments, the degradation moiety has the following structure:

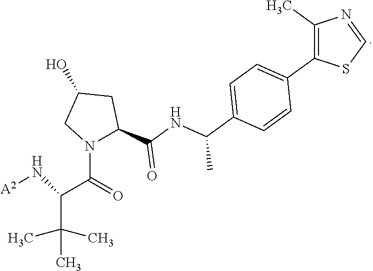

In some embodiments, the degradation moiety has the following structure:

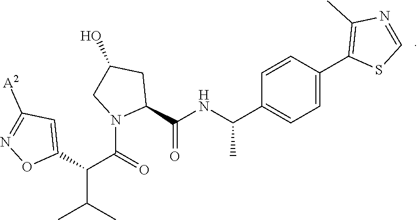

In some embodiments, the degradation moiety is

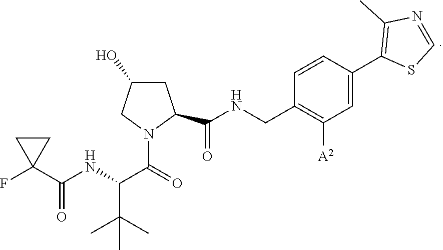

In some embodiments, the degradation moiety includes the structure of

In some embodiments, the degrader moiety includes the structure of Formula D:

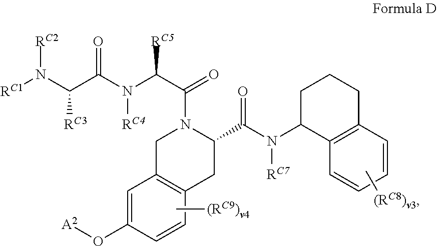

where

A² is a bond between B and the linker;

each of R^{C1}, R^{C2}, and R^{C7} is, independently, H, optionally substituted C₁-C₆ alkyl, or optionally substituted C₁-C₆ heteroalkyl;

R^{C3} is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;

R^{C5} is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆alkyl C₆-C₁₀ aryl;

v3 is 0, 1, 2, 3, or 4;

each R^{C8} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;

v4 is 0, 1, 2, 3, or 4; and

each R^{C9} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino, or a pharmaceutically acceptable salt thereof.

In some embodiments, the degradation moiety includes the structure of

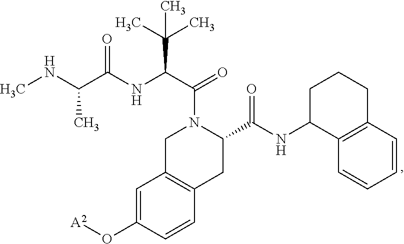

or derivative or analog thereof.

In some embodiments, the degradation moiety includes the structure of Formula E:

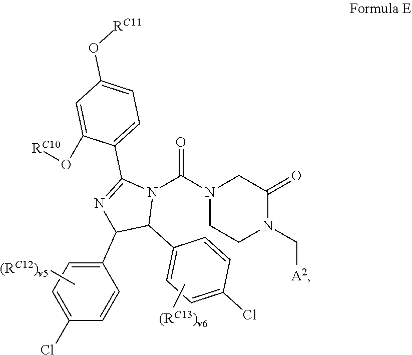

where

A² is a bond between B and the linker;

each of R^{C10} and R^{C11} is, independently, H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;

v5 is 0, 1, 2, 3, or 4;

each R^{C12} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;

v6 is 0, 1, 2, 3, or 4; and

each R²¹ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino, or a pharmaceutically acceptable salt thereof.

In some embodiments, the degradation moiety includes the structure of

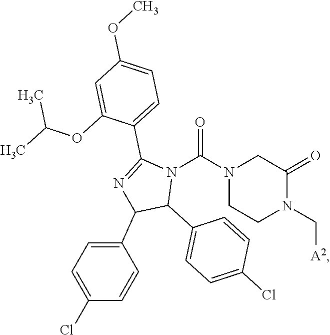

or derivative or analog thereof.

In some embodiments, the degradation moiety includes the structure of Formula FA:

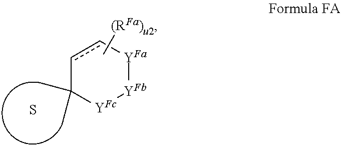

where

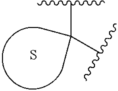

is

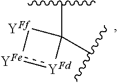

or a bicyclic moiety which is substituted with A² and substituted with one or more groups independently selected from H, R^{FF1}, and oxo;

[symbol] is a single bond or a double bond;

u2 is 0, 1, 2, or 3;

A² is a bond between the degrader and the linker;

Y^{Fa} is CR^{Fb}R^{Fc}, C═O, C═S, C═CH₂, SO₂, S(O), P(O)Oalkyl, P(O)NHalkyl, P(O)N(alkyl)₂, P(O)alkyl, P(O)OH, P(O)NH₂;

Y^{Fb} is NH, NR^{FF1}, CH₂, CHR^{FF1}, C(R^{FF1})₂, O, or S;

Y^{Fc} is CR^{Fd}R^{Fe}, C═O, C═S, C═CH₂, SO₂, S(O), P(O)Oalkyl, P(O)NHalkyl, P(O)N(alkyl)₂, P(O)alkyl, P(O)OH, P(O)NH₂;

each of R^{Fb}, R^{Fc}, R^{Fd}, and R^{Fe} is, independently, H, alkyl, aliphatic, heteroaliphatic, aryl, heteroaryl, carbocyclyl, hydroxyl, alkoxy, amino, —NHalkyl, or —Nalkyl₂;

or R^{Fb} and R^{Fc}, together with the carbon atom to which each is attached, combine to form a 3-, 4-, 5-, or 6-membered spirocarbocyclylene, or a 4-, 5-, or 6-membered spiroheterocyclylene including 1 or 2 heteroatoms selected from N and O;

or R^{Fd} and R^{Fe}, together with the carbon atom to which each is attached, combine to form a 3-, 4-, 5-, or 6-membered spirocarbocyclylene, or a 4-, 5-, or 6-membered spiroheterocyclylene including 1 or 2 heteroatoms selected from N and O; and

or R^{Fd} and R^{Fb}, together with the carbon atoms to which each is attached, combine to form a 1, 2, 3, or 4 carbon bridged ring;

each of Y^{Fd} and Y^{Ff} is, independently, CH₂, CHR^{FF2}, C(R^{FF2})₂, C(O), N, NH, NR^{FF3}, O, S or S(O);

Y^{Fe} is a bond or a divalent moiety attached to Y^{Fd} and Y^{Ff} that contains 1 to 5 contiguous carbon atoms that form a 3 to 8-membered ring,
where 1, 2, or 3 carbon atoms can be replaced with a nitrogen, oxygen, or sulfur atom;
where one of the ring atoms is substituted with A² and the others are substituted with one or more groups independently selected from H and R^{FF1}; and
where the contiguous atoms of Y^{Fe} can be attached rough a single or double bond;

each R^{FF1} is, independently, H, alkyl, alkenyl, alkynyl, aliphatic, heteroaliphatic, carbocyclyl, halogen, hydroxyl, amino, cyano, alkoxy, aryl, heteroaryl, heterocyclyl alkylamino, alkylhydroxyl, or haloalkyl;

each R^{FF2} is, independently, alkyl, alkene, alkyne, halogen, hydroxyl, alkoxy, azide, amino, —C(O)H, —C(O)OH, —C(O)(aliphatic, including alkyl), —C(O)O(aliphatic, including alkyl), —NH(aliphatic, including alkyl), —N(aliphatic including alkyl)(aliphatic including alkyl), —NHSO₂alkyl, —N(alkyl)SO₂alkyl, —NHSO₂aryl, —N(alkyl)SO₂aryl, —NHSO₂alkenyl, —N(alkyl)SO₂alkenyl, —NHSO₂alkynyl, —N(alkyl)SO₂alkynyl, aliphatic, heteroaliphatic, aryl, heteroaryl, hetercyclic, carbocyclic, cyano, nitro, nitroso, —SH, —Salkyl, or haloalkyl; and

R^{FF3} is alkyl, alkenyl, alkynyl, —C(O)H, —C(O)OH, —C(O)alkyl, or —C(O)Oalkyl,

where if Y^{Fd} or Y^{Ff} is substituted with A², then Y^{Fe} is a bond, or a pharmaceutically acceptable salt thereof.

In some embodiments, the degradation moiety includes the structure of Formula FA1:

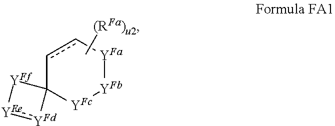

or a pharmaceutically acceptable salt thereof.

In some embodiments, the degradation moiety includes the structure of Formula FB:

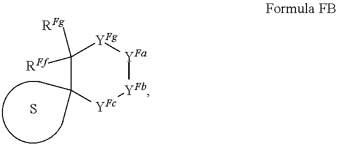

where

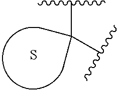

is

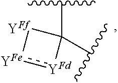

or a bicyclic moiety which is substituted with A² and substituted with one or more groups independently selected from H, R^{FF1}, and oxo;

A² is a bond between the degrader and the linker;

Y^{Fa} is CR^{Fb}R^{Fc}, C═O, C═S, C═CH₂, SO₂, S(O), P(O)Oalkyl, P(O)NHalkyl, P(O)N(alkyl)₂, P(O)alkyl, P(O)OH, P(O)NH₂;

each of Y^{Fb} and Y^{Fg} is, independently, NH, NR^{FF1}, CH₂, CHR^{FF1}, C(R^{FF1})₂, O, or S;

Y^{Fc} is CR^{Fd}R^{Fe}, C═O, C═S, C═CH₂, SO₂, S(O), P(O)Oalkyl, P(O)NHalkyl, P(O)N(alkyl)₂, P(O)alkyl, P(O)OH, P(O)NH₂;

each of R^{Fb}, R^{Fc}, R^{Fd}, R^{Fe}, R^{Ff} and R^{Fg} is, independently, H, alkyl, aliphatic, heteroaliphatic, aryl, heteroaryl, carbocyclyl, hydroxyl, alkoxy, amino, —NHalkyl, or —Nalkyl₂;

or R^{Fb} and R^{Fc}, together with the carbon atom to which each is attached, combine to form a 3-, 4-, 5-, or 6-membered spirocarbocyclylene, or a 4-, 5-, or 6-membered spiroheterocyclylene including 1 or 2 heteroatoms selected from N and O;

or R^{Fd} and R^{Fe}, together with the carbon atom to which each is attached, combine to form a 3-, 4-, 5-, or 6-membered spirocarbocyclylene, or a 4-, 5-, or 6-membered spiroheterocyclylene including 1 or 2 heteroatoms selected from N and O;

or R^{Ff} and R^{Fg}, together with the carbon atom to which each is attached, combine to form a 3-, 4-, 5-, or 6-membered spirocarbocyclylene, or a 4-, 5-, or 6-membered spiroheterocyclylene including 1 or 2 heteroatoms selected from N and O;

or R^{Fd} and R^{Fb}, together with the carbon atoms to which each is attached, combine to form a 1, 2, 3, or 4 carbon bridged ring;

or R^{Fd} and R^{Ff}, together with the carbon atoms to which each is attached, combine to form a 1, 2, 3, or 4 carbon bridged ring;

or R^{Fb} and R^{Fg}, together with the carbon atoms to which each is attached, combine to form a 1, 2, 3, or 4 carbon bridged ring;

each of Y^{Fd} and Y^{Ff} is, independently, CH₂, CHR^{FF2}, C(R^{FF2})₂, C(O), N, NH, NR^{FF3}, O, S, or S(O);

Y^{Fe} is a bond or a divalent moiety attached to Y^{Fd} and Y^{Ff} that contains 1 to 5 contiguous carbon atoms that form a 3 to 8-membered ring,
where 1, 2, or 3 carbon atoms can be replaced with a nitrogen, oxygen, or sulfur atom;
where one of the ring atoms is substituted with A² and the others are substituted with one or more groups independently selected from H and R^{FF1}; and
where the contiguous atoms of Y^{Fe} can be attached through a single or double bond;

each R^{FF1} is, independently, H, alkyl, alkenyl, alkynyl, aliphatic, heteroaliphatic, carbocyclyl, halogen, hydroxyl, amino, cyano, alkoxy, aryl, heteroaryl, heterocyclyl, alkylamino, alkylhydroxyl, or haloalkyl;

each R^{FF2} is, independently, alkyl, alkene, alkyne, halogen, hydroxyl, alkoxy, azide, amino, —C(O)H, —C(O)OH, —C(O)(aliphatic, including alkyl), —C(O)O(aliphatic, including alkyl), —NH(aliphatic, including alkyl), —N(aliphatic including alkyl)(aliphatic including alkyl), —NHSO₂alkyl, —N(alkyl)SO₂alkyl, —NHSO₂aryl, —N(alkyl)SO₂aryl, —NHSO₂alkenyl, —N(alkyl)SO₂alkenyl, —NHSO₂alkynyl, —N(alkyl)SO₂alkynyl, aliphatic, heteroaliphatic, aryl, heteroaryl, hetercyclic, carbocyclic, cyano, nitro, nitroso, —SH, —Salkyl, or haloalkyl; and

R^{FF3} is alkyl, alkenyl, alkynyl, —C(O)H, —C(O)OH, —C(O)alkyl, or —C(O)Oalkyl,

where if Y^{Fd} or Y^{Ff} is substituted with A², then Y^{Fe} is a bond, or a pharmaceutically acceptable salt thereof.

In some embodiments, the degradation moiety includes the structure of Formula FBI:

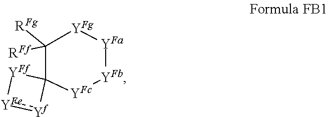

or a pharmaceutically acceptable salt thereof.

In some embodiments, the degradation moiety includes the structure of Formula F1:

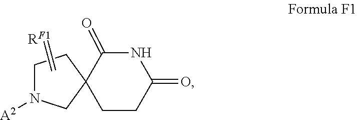

where A² is a bond between the degrader and the linker; and R^{F1} is absent or O, or a pharmaceutically acceptable salt thereof.

In some embodiments, R^{F1} is absent. In some embodiments, R^{F1} is O.

In some embodiments, the degradation moiety includes the structure of

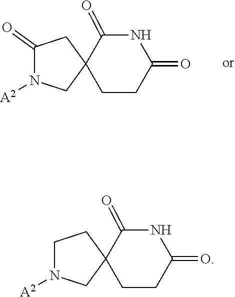

In some embodiments, the degradation moiety includes the structure Formula F2:

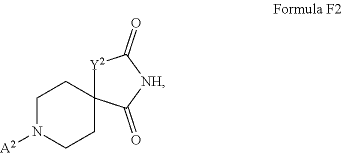

where A² is a bond between the degrader and the linker; and Y² is CH₂ or NH, or a pharmaceutically acceptable salt thereof.

In some embodiments, Y² is NH. In some embodiments, Y² is CH₂.

In some embodiments, the degradation moiety includes the structure of

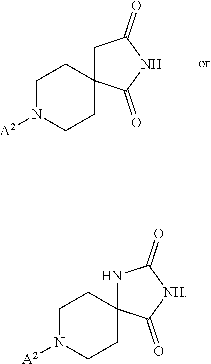

In some embodiments, the degradation moiety includes the structure Formula G:

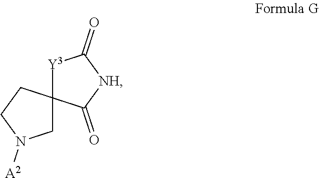

where A² is a bond between the degrader and the linker; and Y³ is CH₂ or NH, or a pharmaceutically acceptable salt thereof.

In some embodiments, Y³ is NH. In some embodiments, Y³ is CH₂.

In some embodiments, the degradation moiety includes the structure of

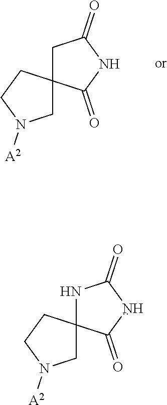

The degradation moiety may also include structures found in, e.g., WO2017/197036; WO2019/204354, WO2019/236483, WO2020/010177; and WO2020/010227, the structures of which are herein incorporated by reference.

In some embodiments, the degradation moiety includes the structure of

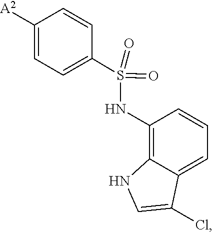

where A² is a bond between the degradation moiety and the linker or is a derivative or an analog thereof.

In some embodiments, the compound has the structure of any one of compounds 1-75 in Table 1, or a pharmaceutically acceptable salt thereof. In some embodiments, the compound has the structure of any one of compounds 76-104 in Table 2, or a pharmaceutically acceptable salt thereof.

In an aspect, the invention features a compound having the structure of any one of compounds 1-75 in Table 1, or a pharmaceutically acceptable salt thereof.

In an aspect, the invention features a compound having the structure of any one of compounds 105-272 in Table 2, or a pharmaceutically acceptable salt thereof.

In another aspect, the invention features a compound having the structure of any one of compounds 76-104 in Table 2, or a pharmaceutically acceptable salt thereof.

TABLE 1
Compounds of the Invention

| Comp. | |
|---|---|
| No. | Structure |
| 1 | 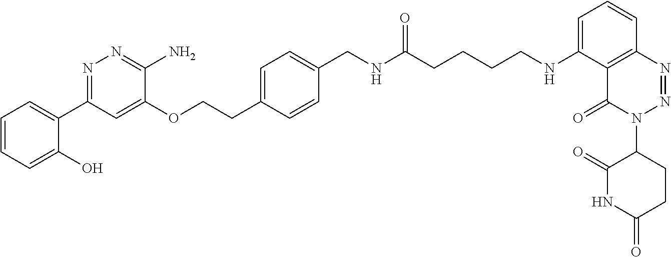 [SMILES: Nc1nnc(-c2ccccc2O)cc1OCCc1ccc(CNC(=O)CCCCNc2cccc3nnn(C4CCC(=O)NC4=O)c(=O)c23)cc1] |
| 2 | 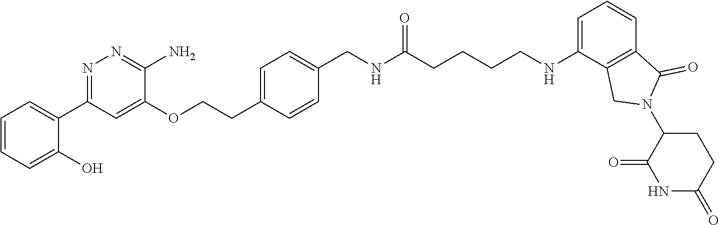 [SMILES: Nc1nnc(-c2ccccc2O)cc1OCCc1ccc(CNC(=O)CCCCNc2cccc3c2CN(C2CCC(=O)NC2=O)C3=O)cc1] |
| 3 | 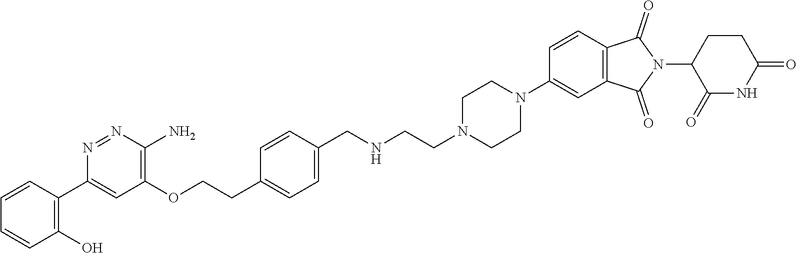 [SMILES: Nc1nnc(-c2ccccc2O)cc1OCCc1ccc(CNCCN2CCN(c3ccc4c(c3)C(=O)N(C3CCC(=O)NC3=O)C4=O)CC2)cc1] |
| 4 | 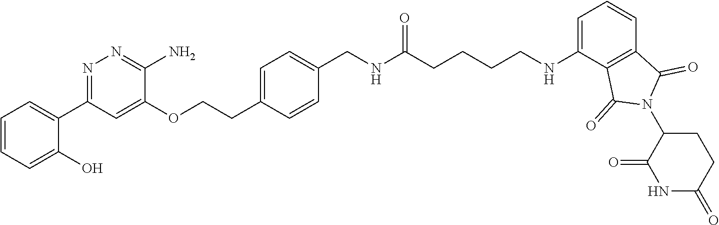 [SMILES: Nc1nnc(-c2ccccc2O)cc1OCCc1ccc(CNC(=O)CCCCNc2cccc3c2C(=O)N(C2CCC(=O)NC2=O)C3=O)cc1] |
| 5 | 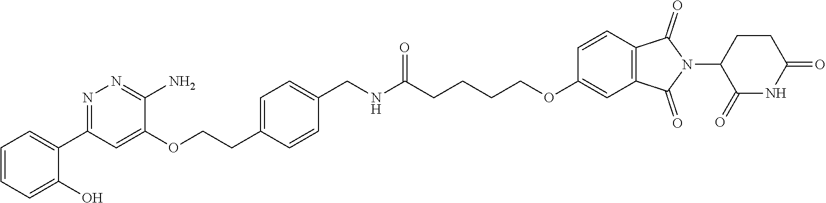 [SMILES: Nc1nnc(-c2ccccc2O)cc1OCCc1ccc(CNC(=O)CCCCOc2ccc3c(c2)C(=O)N(C2CCC(=O)NC2=O)C3=O)cc1] |
| 6 | 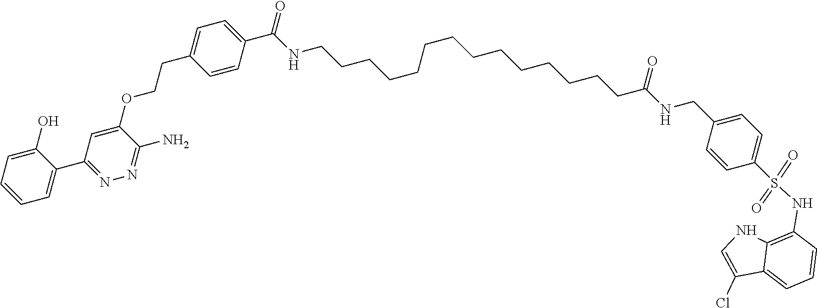 [SMILES: Nc1nnc(-c2ccccc2O)cc1OCCc1ccc(C(=O)NCCCCCCCCCCCCCCC(=O)NCc2ccc(S(=O)(=O)Nc3cccc4c(Cl)c[nH]c34)cc2)cc1] |
| 7 | 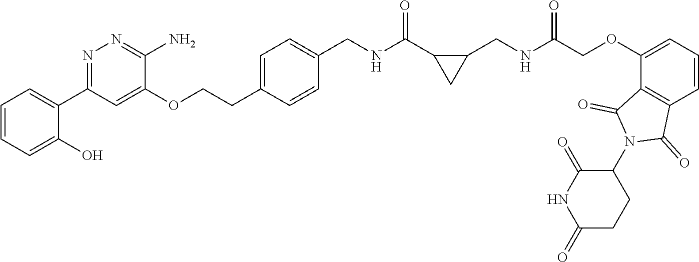 [SMILES: Nc1nnc(-c2ccccc2O)cc1OCCc1ccc(CNC(=O)C2CC2CNC(=O)COc2cccc3c2C(=O)N(C2CCC(=O)NC2=O)C3=O)cc1] |
| 8 | 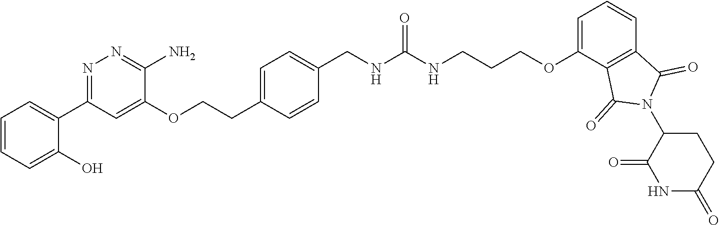 [SMILES: Nc1nnc(-c2ccccc2O)cc1OCCc1ccc(CNC(=O)NCCCOc2cccc3c2C(=O)N(C2CCC(=O)NC2=O)C3=O)cc1] |
| 9 | 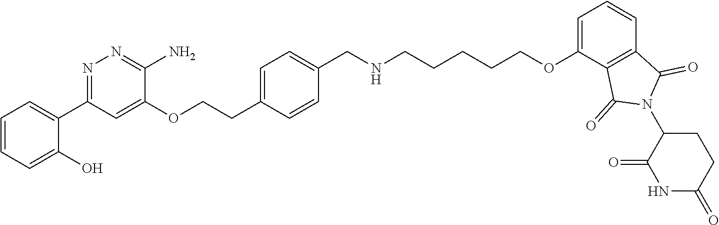 [SMILES: Nc1nnc(-c2ccccc2O)cc1OCCc1ccc(CNCCCCCOc2cccc3c2C(=O)N(C2CCC(=O)NC2=O)C3=O)cc1] |
| 10 | 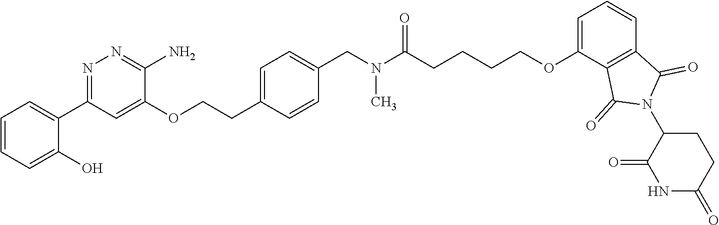 [SMILES: CN(Cc1ccc(CCOc2cc(-c3ccccc3O)nnc2N)cc1)C(=O)CCCCOc1cccc2c1C(=O)N(C1CCC(=O)NC1=O)C2=O] |
| 11 | 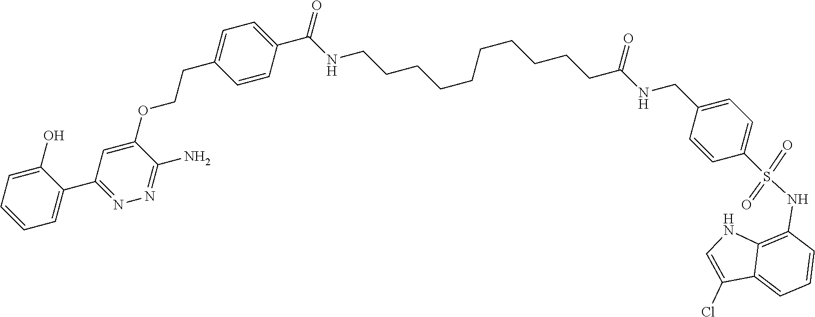 [SMILES: Nc1nnc(-c2ccccc2O)cc1OCCc1ccc(C(=O)NCCCCCCCCCCC(=O)NCc2ccc(S(=O)(=O)Nc3cccc4c(Cl)c[nH]c34)cc2)cc1] |
| 12 | 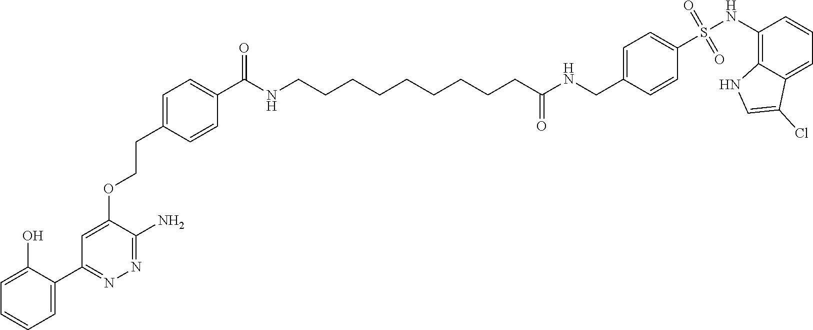 [SMILES: Nc1nnc(-c2ccccc2O)cc1OCCc1ccc(C(=O)NCCCCCCCCCC(=O)NCc2ccc(S(=O)(=O)Nc3cccc4c(Cl)c[nH]c34)cc2)cc1] |
| 13 | 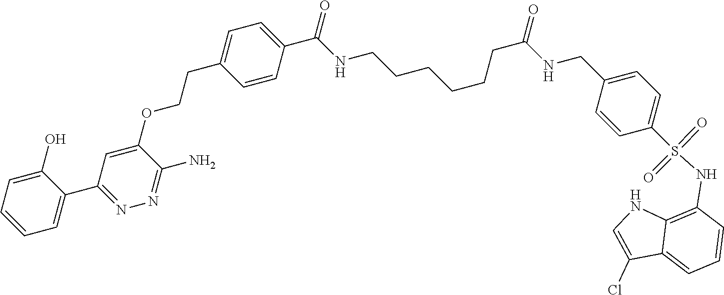 [SMILES: Nc1nnc(-c2ccccc2O)cc1OCCc1ccc(C(=O)NCCCCCCC(=O)NCc2ccc(S(=O)(=O)Nc3cccc4c(Cl)c[nH]c34)cc2)cc1] |
| 14 | 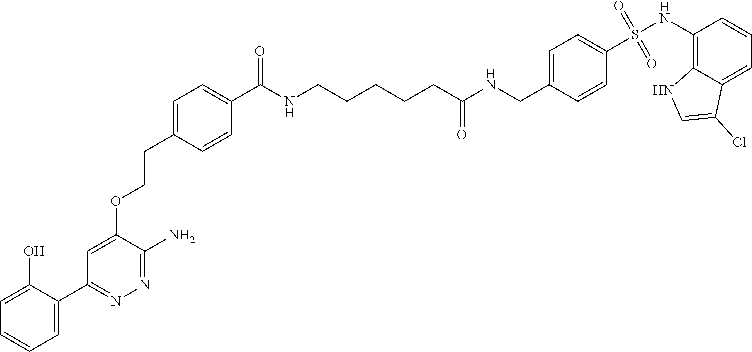 [SMILES: Nc1nnc(-c2ccccc2O)cc1OCCc1ccc(C(=O)NCCCCCC(=O)NCc2ccc(S(=O)(=O)Nc3cccc4c(Cl)c[nH]c34)cc2)cc1] |
| 15 | 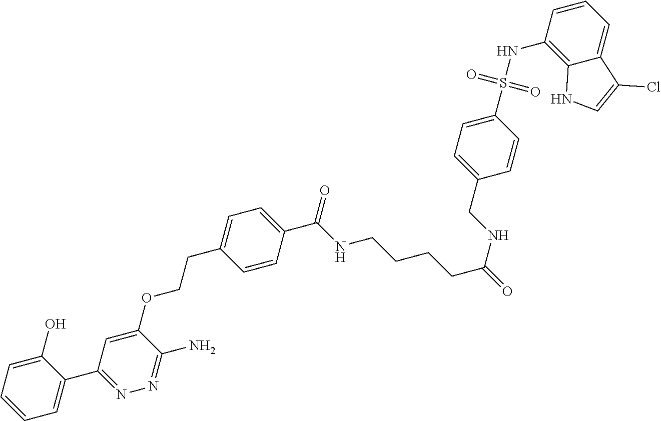 [SMILES: Nc1nnc(-c2ccccc2O)cc1OCCc1ccc(C(=O)NCCCCC(=O)NCc2ccc(S(=O)(=O)Nc3cccc4c(Cl)c[nH]c34)cc2)cc1] |
| 16 | 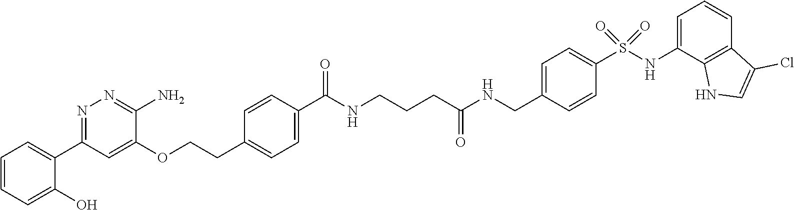 [SMILES: Nc1nnc(-c2ccccc2O)cc1OCCc1ccc(C(=O)NCCCC(=O)NCc2ccc(S(=O)(=O)Nc3cccc4c(Cl)c[nH]c34)cc2)cc1] |
| 17 | 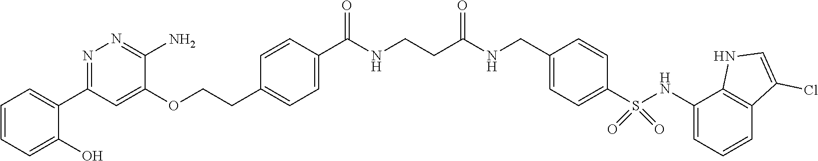 [SMILES: Nc1nnc(-c2ccccc2O)cc1OCCc1ccc(C(=O)NCCC(=O)NCc2ccc(S(=O)(=O)Nc3cccc4c(Cl)c[nH]c34)cc2)cc1] |
| 18 | 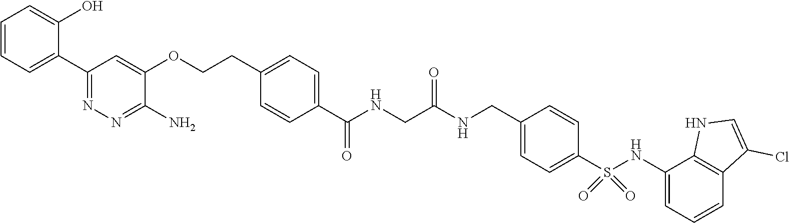 [SMILES: Nc1nnc(-c2ccccc2O)cc1OCCc1ccc(C(=O)NCC(=O)NCc2ccc(S(=O)(=O)Nc3cccc4c(Cl)c[nH]c34)cc2)cc1] |
| 19 | 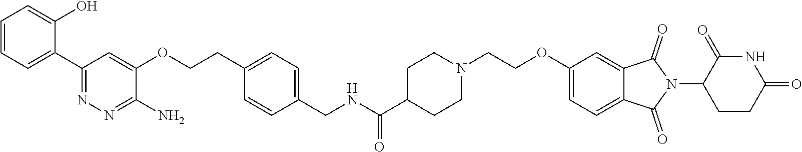 [SMILES: Nc1nnc(-c2ccccc2O)cc1OCCc1ccc(CNC(=O)C2CCN(CCOc3ccc4c(c3)C(=O)N(C3CCC(=O)NC3=O)C4=O)CC2)cc1] |
| 20 | 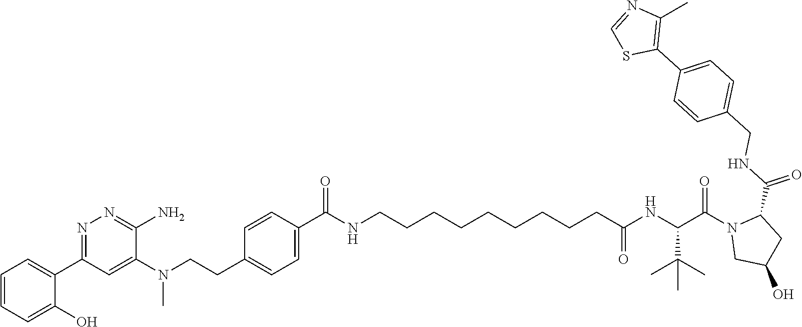 [SMILES: Cc1ncsc1-c1ccc(CNC(=O)[C@@H]2C[C@@H](O)CN2C(=O)[C@@H](NC(=O)CCCCCCCCCNC(=O)c2ccc(CCN(C)c3cc(-c4ccccc4O)nnc3N)cc2)C(C)(C)C)cc1] |
| 21 | 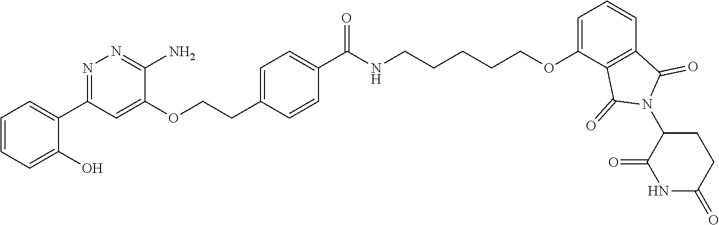 [SMILES: Nc1nnc(-c2ccccc2O)cc1OCCc1ccc(C(=O)NCCCCCOc2cccc3c2C(=O)N(C2CCC(=O)NC2=O)C3=O)cc1] |
| 22 | 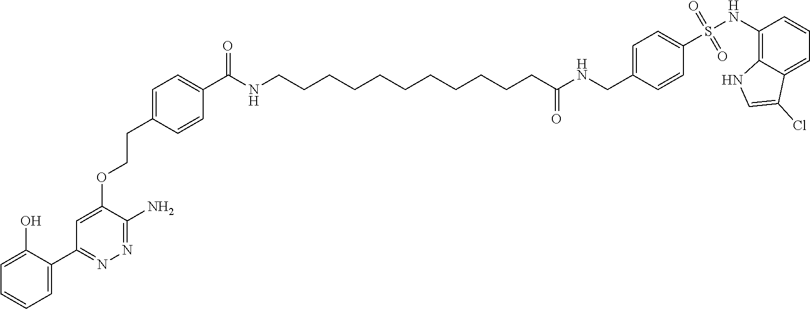 [SMILES: Nc1nnc(-c2ccccc2O)cc1OCCc1ccc(C(=O)NCCCCCCCCCCCC(=O)NCc2ccc(S(=O)(=O)Nc3cccc4c(Cl)c[nH]c34)cc2)cc1] |
| 23 | 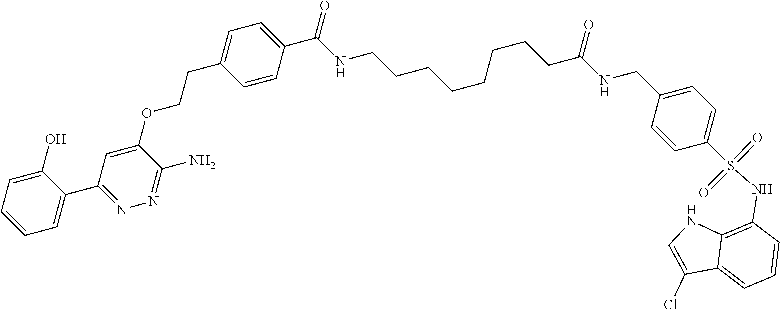 [SMILES: Nc1nnc(-c2ccccc2O)cc1OCCc1ccc(C(=O)NCCCCCCCCC(=O)NCc2ccc(S(=O)(=O)Nc3cccc4c(Cl)c[nH]c34)cc2)cc1] |
| 24 | 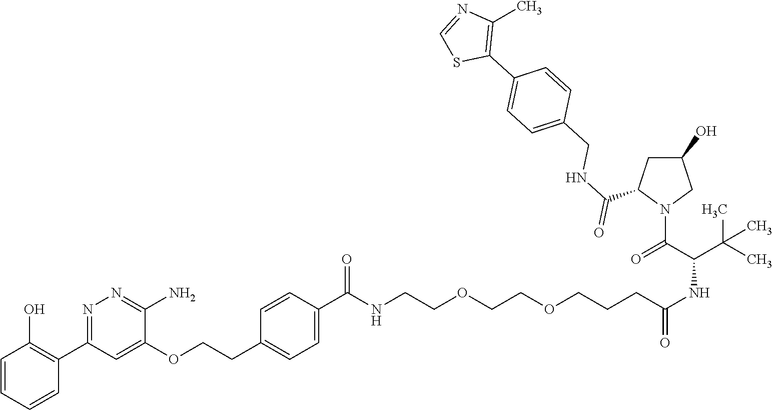 [SMILES: Cc1ncsc1-c1ccc(CNC(=O)[C@@H]2C[C@@H](O)CN2C(=O)[C@@H](NC(=O)CCCOCCOCCNC(=O)c2ccc(CCOc3cc(-c4ccccc4O)nnc3N)cc2)C(C)(C)C)cc1] |
| 25 | 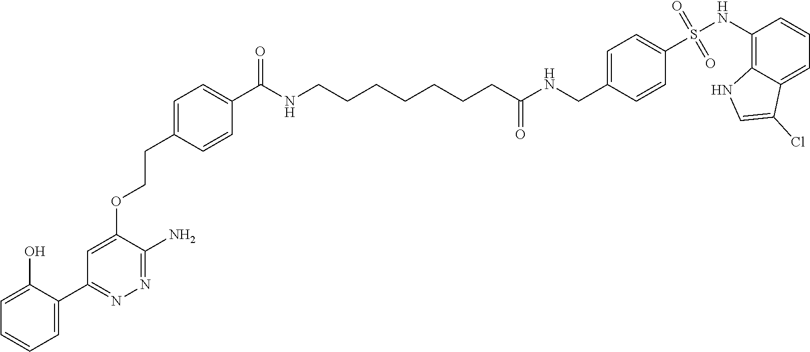 [SMILES: Nc1nnc(-c2ccccc2O)cc1OCCc1ccc(C(=O)NCCCCCCCC(=O)NCc2ccc(S(=O)(=O)Nc3cccc4c(Cl)c[nH]c34)cc2)cc1] |
| 26 | 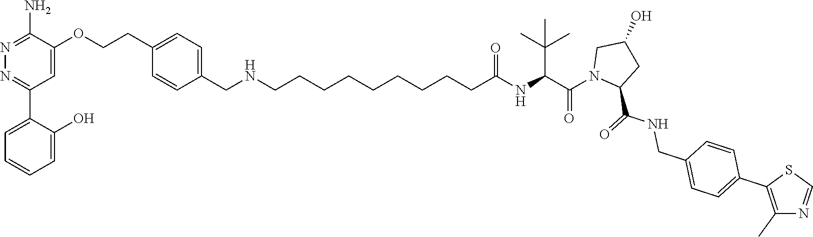 [SMILES: Cc1ncsc1-c1ccc(CNC(=O)[C@@H]2C[C@@H](O)CN2C(=O)[C@@H](NC(=O)CCCCCCCCCNCc2ccc(CCOc3cc(-c4ccccc4O)nnc3N)cc2)C(C)(C)C)cc1] |
| 27 | 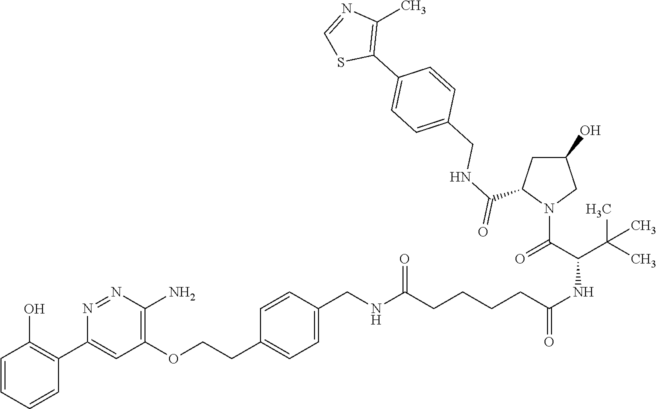 [SMILES: Cc1ncsc1-c1ccc(CNC(=O)[C@@H]2C[C@@H](O)CN2C(=O)[C@@H](NC(=O)CCCCC(=O)NCc2ccc(CCOc3cc(-c4ccccc4O)nnc3N)cc2)C(C)(C)C)cc1] |
| 28 | 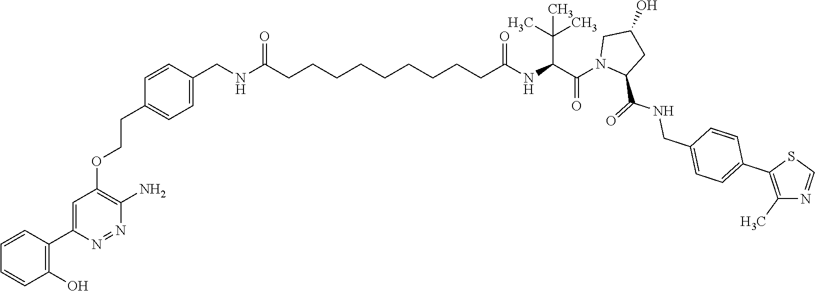 [SMILES: Cc1ncsc1-c1ccc(CNC(=O)[C@@H]2C[C@@H](O)CN2C(=O)[C@@H](NC(=O)CCCCCCCCCC(=O)NCc2ccc(CCOc3cc(-c4ccccc4O)nnc3N)cc2)C(C)(C)C)cc1] |
| 29 | 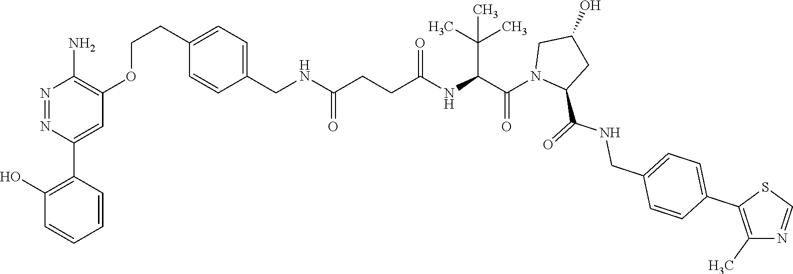 [SMILES: Cc1ncsc1-c1ccc(CNC(=O)[C@@H]2C[C@@H](O)CN2C(=O)[C@@H](NC(=O)CCC(=O)NCc2ccc(CCOc3cc(-c4ccccc4O)nnc3N)cc2)C(C)(C)C)cc1] |
| 30 | 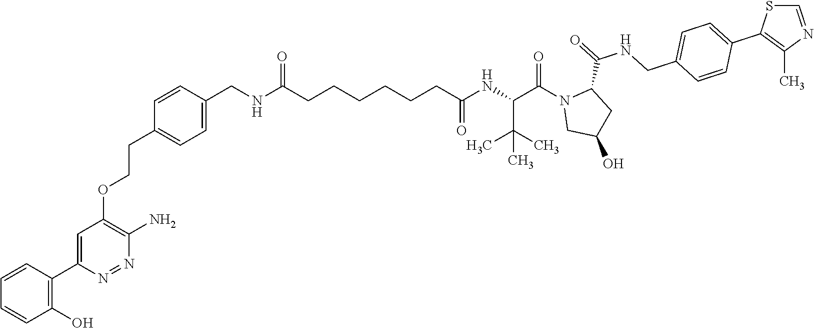 [SMILES: Cc1ncsc1-c1ccc(CNC(=O)[C@@H]2C[C@@H](O)CN2C(=O)[C@@H](NC(=O)CCCCCCC(=O)NCc2ccc(CCOc3cc(-c4ccccc4O)nnc3N)cc2)C(C)(C)C)cc1] |
| 31 | 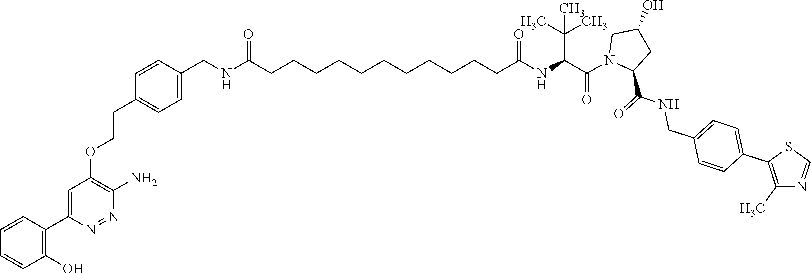 [SMILES: Cc1ncsc1-c1ccc(CNC(=O)[C@@H]2C[C@@H](O)CN2C(=O)[C@@H](NC(=O)CCCCCCCCCCCC(=O)NCc2ccc(CCOc3cc(-c4ccccc4O)nnc3N)cc2)C(C)(C)C)cc1] |
| 32 | 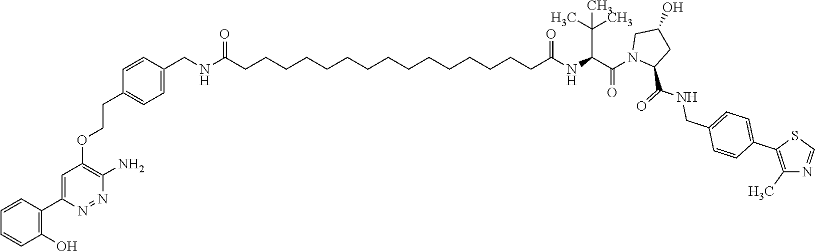 [SMILES: Cc1ncsc1-c1ccc(CNC(=O)[C@@H]2C[C@@H](O)CN2C(=O)[C@@H](NC(=O)CCCCCCCCCCCCCCCC(=O)NCc2ccc(CCOc3cc(-c4ccccc4O)nnc3N)cc2)C(C)(C)C)cc1] |
| 33 | 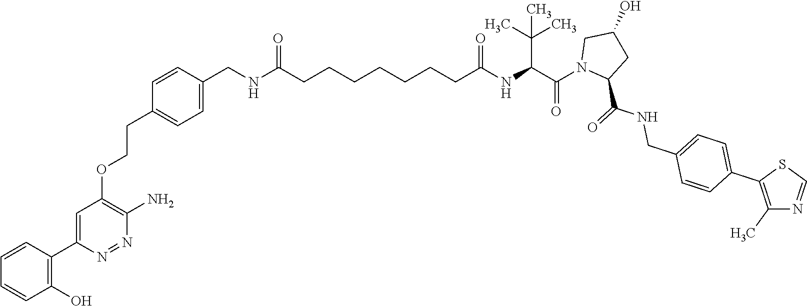 [SMILES: Cc1ncsc1-c1ccc(CNC(=O)[C@@H]2C[C@@H](O)CN2C(=O)[C@@H](NC(=O)CCCCCCCC(=O)NCc2ccc(CCOc3cc(-c4ccccc4O)nnc3N)cc2)C(C)(C)C)cc1] |
| 34 | 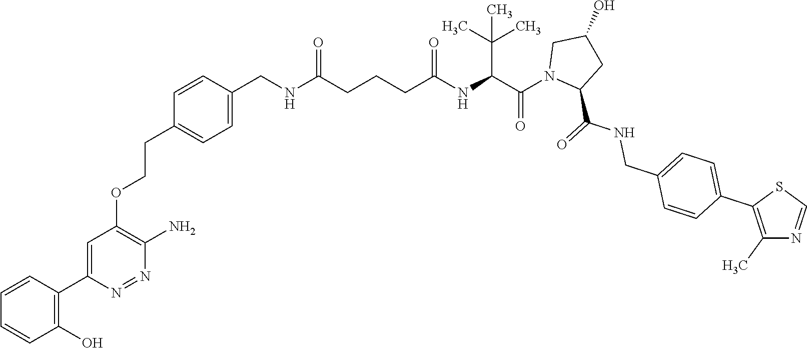 [SMILES: Cc1ncsc1-c1ccc(CNC(=O)[C@@H]2C[C@@H](O)CN2C(=O)[C@@H](NC(=O)CCCC(=O)NCc2ccc(CCOc3cc(-c4ccccc4O)nnc3N)cc2)C(C)(C)C)cc1] |
| 35 | 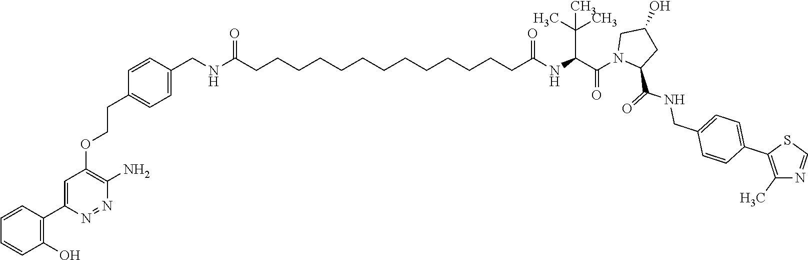 [SMILES: Cc1ncsc1-c1ccc(CNC(=O)[C@@H]2C[C@@H](O)CN2C(=O)[C@@H](NC(=O)CCCCCCCCCCCCCC(=O)NCc2ccc(CCOc3cc(-c4ccccc4O)nnc3N)cc2)C(C)(C)C)cc1] |
| 36 | 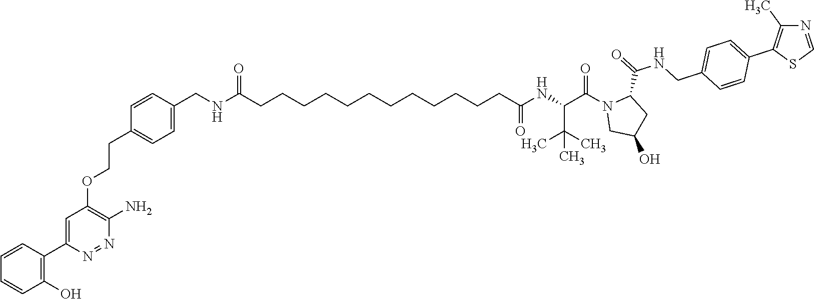 [SMILES: Cc1ncsc1-c1ccc(CNC(=O)[C@@H]2C[C@@H](O)CN2C(=O)[C@@H](NC(=O)CCCCCCCCCCCCC(=O)NCc2ccc(CCOc3cc(-c4ccccc4O)nnc3N)cc2)C(C)(C)C)cc1] |
| 37 | 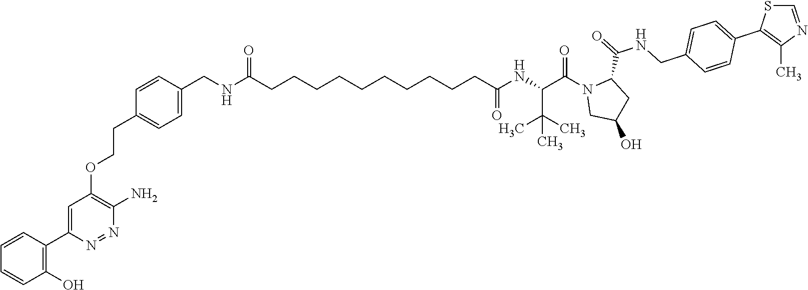 [SMILES: Cc1ncsc1-c1ccc(CNC(=O)[C@@H]2C[C@@H](O)CN2C(=O)[C@@H](NC(=O)CCCCCCCCCCC(=O)NCc2ccc(CCOc3cc(-c4ccccc4O)nnc3N)cc2)C(C)(C)C)cc1] |
| 38 | 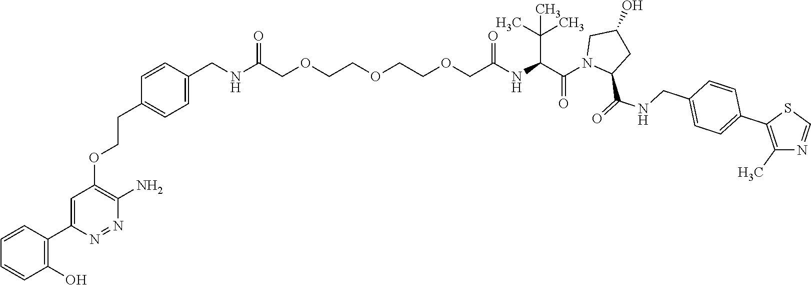 [SMILES: Cc1ncsc1-c1ccc(CNC(=O)[C@@H]2C[C@@H](O)CN2C(=O)[C@@H](NC(=O)COCCOCCOCC(=O)NCc2ccc(CCOc3cc(-c4ccccc4O)nnc3N)cc2)C(C)(C)C)cc1] |
| 39 | 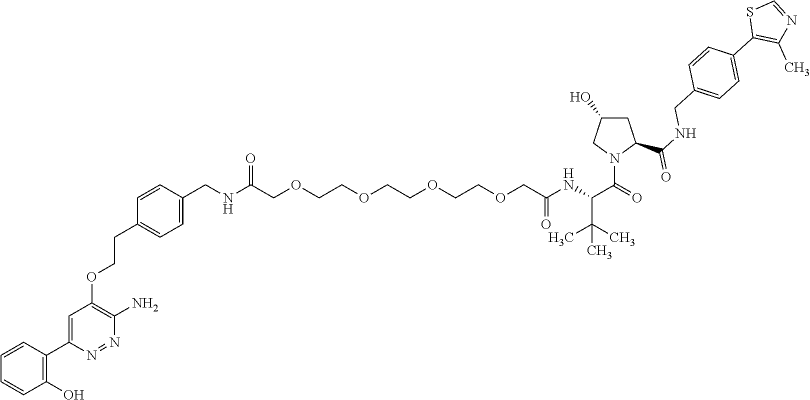 [SMILES: Cc1ncsc1-c1ccc(CNC(=O)[C@@H]2C[C@@H](O)CN2C(=O)[C@@H](NC(=O)COCCOCCOCCOCC(=O)NCc2ccc(CCOc3cc(-c4ccccc4O)nnc3N)cc2)C(C)(C)C)cc1] |
| 40 | 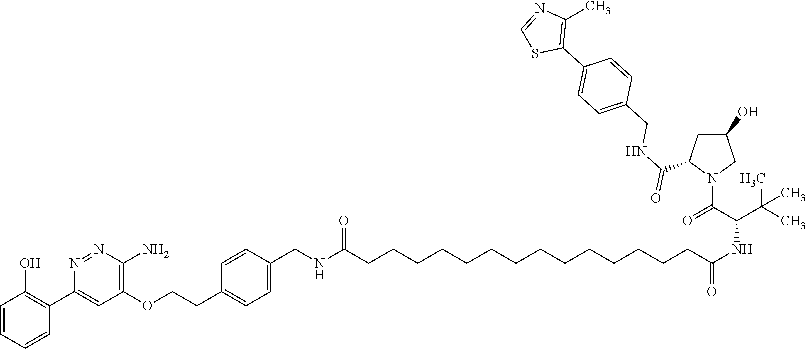 [SMILES: Cc1ncsc1-c1ccc(CNC(=O)[C@@H]2C[C@@H](O)CN2C(=O)[C@@H](NC(=O)CCCCCCCCCCCCCCC(=O)NCc2ccc(CCOc3cc(-c4ccccc4O)nnc3N)cc2)C(C)(C)C)cc1] |
| 41 | 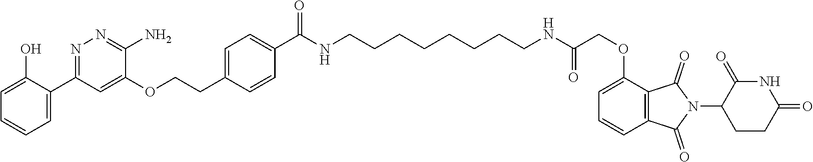 [SMILES: Nc1nnc(-c2ccccc2O)cc1OCCc1ccc(C(=O)NCCCCCCCCNC(=O)COc2cccc3c2C(=O)N(C2CCC(=O)NC2=O)C3=O)cc1] |
| 42 | 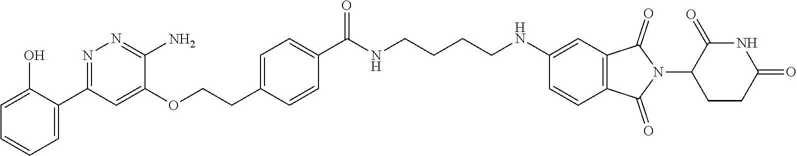 [SMILES: Nc1nnc(-c2ccccc2O)cc1OCCc1ccc(C(=O)NCCCCNc2ccc3c(c2)C(=O)N(C2CCC(=O)NC2=O)C3=O)cc1] |
| 43 | 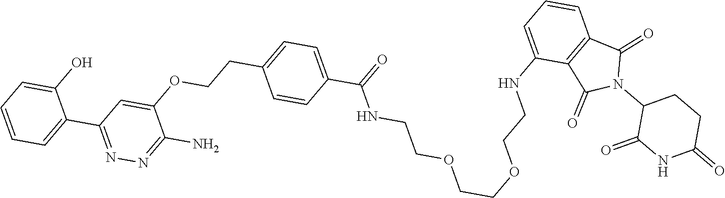 [SMILES: Nc1nnc(-c2ccccc2O)cc1OCCc1ccc(C(=O)NCCOCCOCCNc2cccc3c2C(=O)N(C2CCC(=O)NC2=O)C3=O)cc1] |
| 44 | 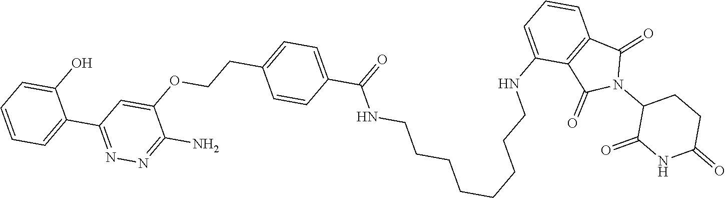 [SMILES: Nc1nnc(-c2ccccc2O)cc1OCCc1ccc(C(=O)NCCCCCCCCNc2cccc3c2C(=O)N(C2CCC(=O)NC2=O)C3=O)cc1] |
| 45 | 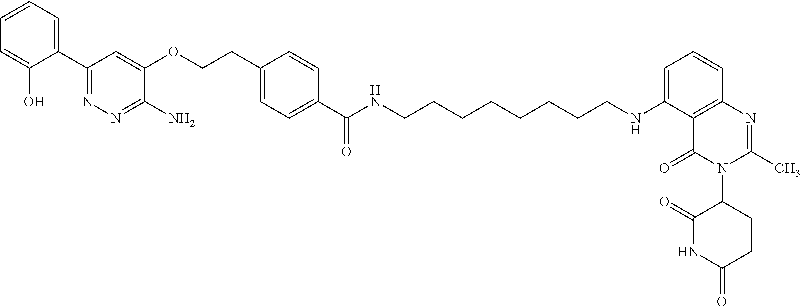 [SMILES: Cc1nc2cccc(NCCCCCCCCNC(=O)c3ccc(CCOc4cc(-c5ccccc5O)nnc4N)cc3)c2c(=O)n1C1CCC(=O)NC1=O] |
| 46 | 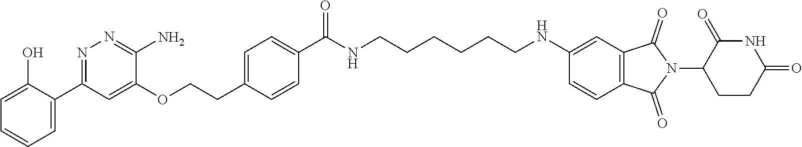 [SMILES: Nc1nnc(-c2ccccc2O)cc1OCCc1ccc(C(=O)NCCCCCCNc2ccc3c(c2)C(=O)N(C2CCC(=O)NC2=O)C3=O)cc1] |
| 47 | 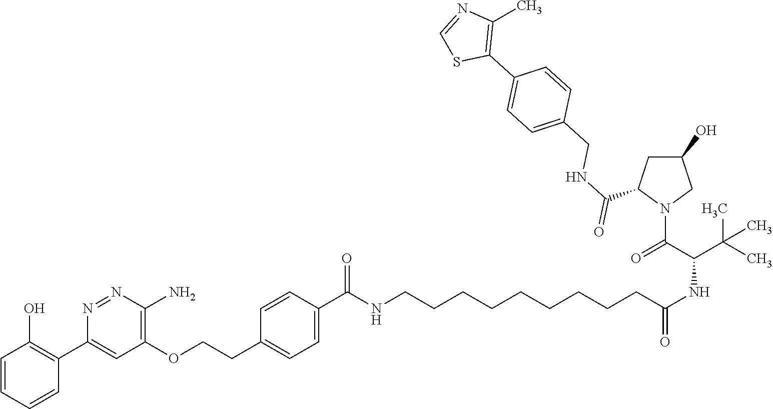 [SMILES: Cc1ncsc1-c1ccc(CNC(=O)[C@@H]2C[C@@H](O)CN2C(=O)[C@@H](NC(=O)CCCCCCCCCNC(=O)c2ccc(CCOc3cc(-c4ccccc4O)nnc3N)cc2)C(C)(C)C)cc1] |
| 48 | 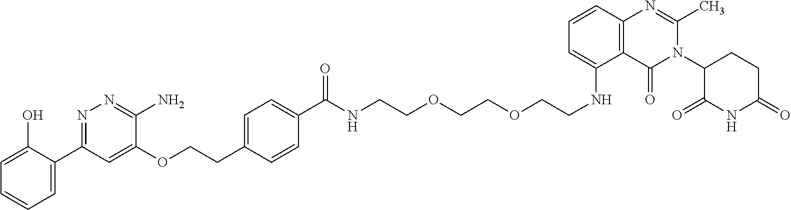 [SMILES: Cc1nc2cccc(NCCOCCOCCNC(=O)c3ccc(CCOc4cc(-c5ccccc5O)nnc4N)cc3)c2c(=O)n1C1CCC(=O)NC1=O] |
| 49 | 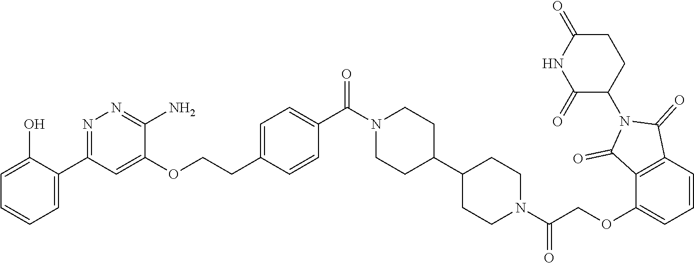 [SMILES: Nc1nnc(-c2ccccc2O)cc1OCCc1ccc(C(=O)N2CCC(C3CCN(C(=O)COc4cccc5c4C(=O)N(C4CCC(=O)NC4=O)C5=O)CC3)CC2)cc1] |
| 50 | 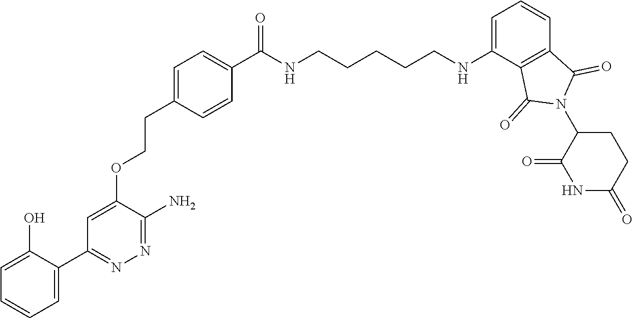 [SMILES: Nc1nnc(-c2ccccc2O)cc1OCCc1ccc(C(=O)NCCCCCNc2cccc3c2C(=O)N(C2CCC(=O)NC2=O)C3=O)cc1] |
| 51 | 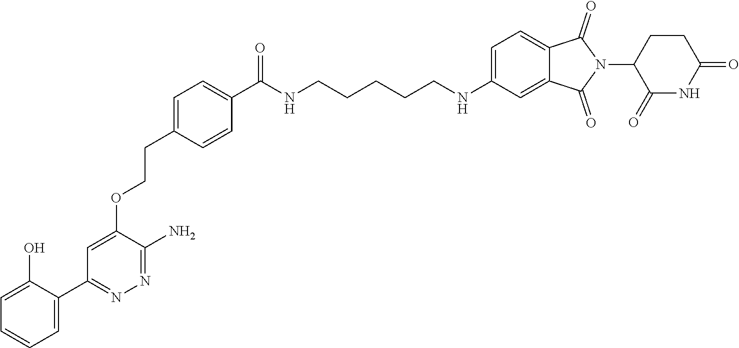 [SMILES: Nc1nnc(-c2ccccc2O)cc1OCCc1ccc(C(=O)NCCCCCNc2ccc3c(c2)C(=O)N(C2CCC(=O)NC2=O)C3=O)cc1] |
| 52 | 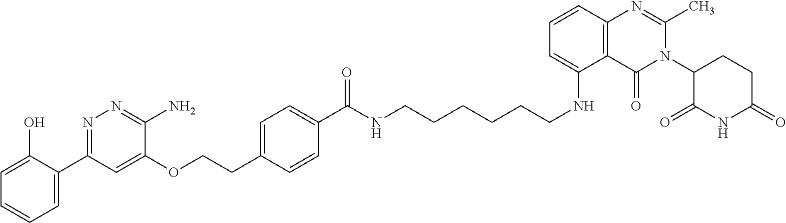 [SMILES: Cc1nc2cccc(NCCCCCCNC(=O)c3ccc(CCOc4cc(-c5ccccc5O)nnc4N)cc3)c2c(=O)n1C1CCC(=O)NC1=O] |
| 53 | 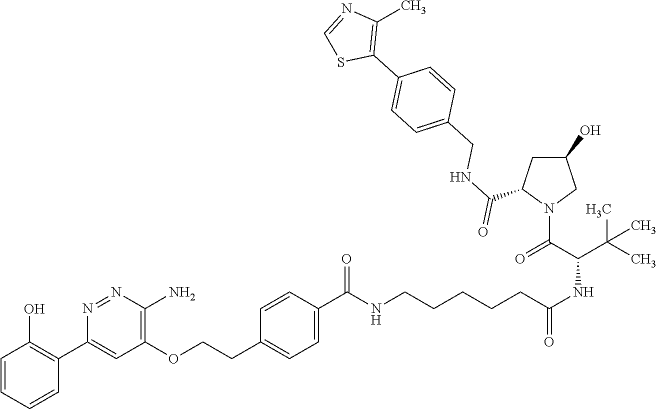 [SMILES: Cc1ncsc1-c1ccc(CNC(=O)[C@@H]2C[C@@H](O)CN2C(=O)[C@@H](NC(=O)CCCCCNC(=O)c2ccc(CCOc3cc(-c4ccccc4O)nnc3N)cc2)C(C)(C)C)cc1] |
| 54 | 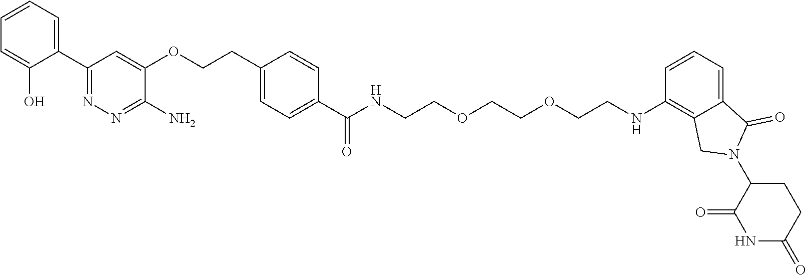 [SMILES: Nc1nnc(-c2ccccc2O)cc1OCCc1ccc(C(=O)NCCOCCOCCNc2cccc3c2CN(C2CCC(=O)NC2=O)C3=O)cc1] |
| 55 | 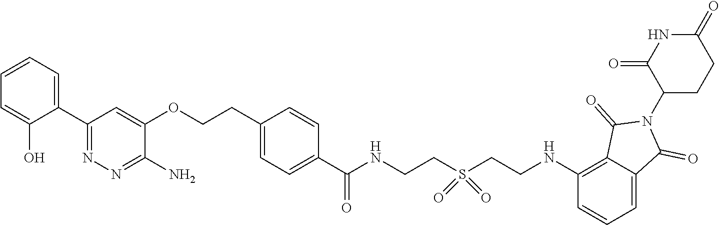 [SMILES: Nc1nnc(-c2ccccc2O)cc1OCCc1ccc(C(=O)NCCS(=O)(=O)CCNc2cccc3c2C(=O)N(C2CCC(=O)NC2=O)C3=O)cc1] |
| 56 | 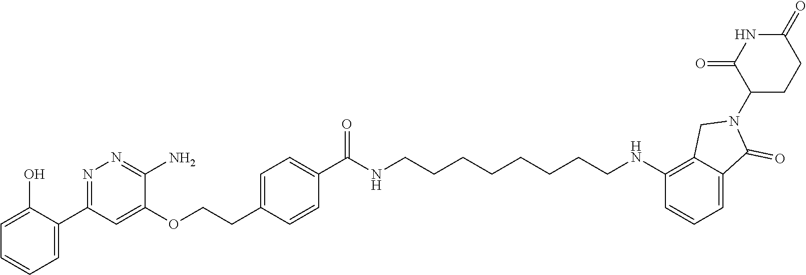 [SMILES: Nc1nnc(-c2ccccc2O)cc1OCCc1ccc(C(=O)NCCCCCCCCNc2cccc3c2CN(C2CCC(=O)NC2=O)C3=O)cc1] |
| 57 | 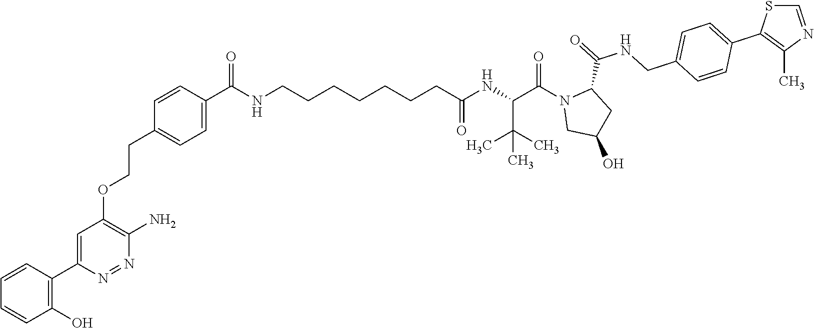 [SMILES: Cc1ncsc1-c1ccc(CNC(=O)[C@@H]2C[C@@H](O)CN2C(=O)[C@@H](NC(=O)CCCCCCCNC(=O)c2ccc(CCOc3cc(-c4ccccc4O)nnc3N)cc2)C(C)(C)C)cc1] |
| 58 | 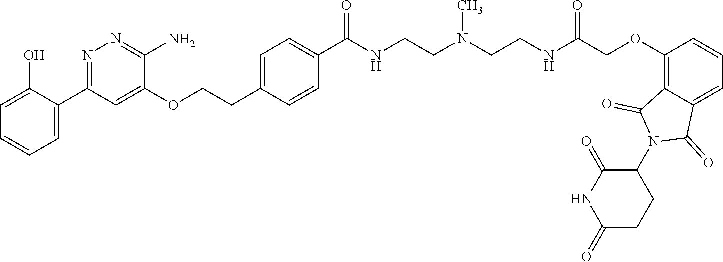 [SMILES: CN(CCNC(=O)COc1cccc2c1C(=O)N(C1CCC(=O)NC1=O)C2=O)CCNC(=O)c1ccc(CCOc2cc(-c3ccccc3O)nnc2N)cc1] |
| 59 | 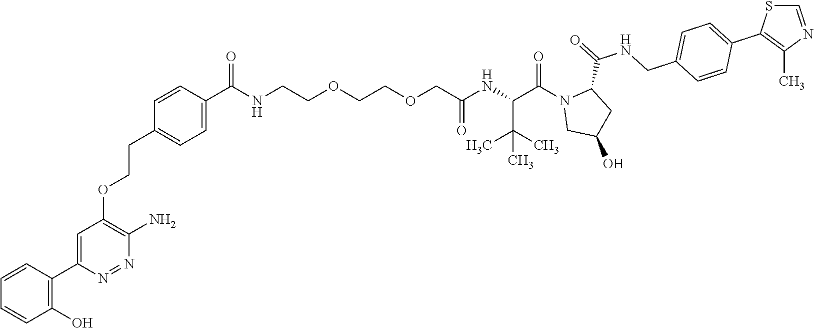 [SMILES: Cc1ncsc1-c1ccc(CNC(=O)[C@@H]2C[C@@H](O)CN2C(=O)[C@@H](NC(=O)COCCOCCNC(=O)c2ccc(CCOc3cc(-c4ccccc4O)nnc3N)cc2)C(C)(C)C)cc1] |
| 60 | 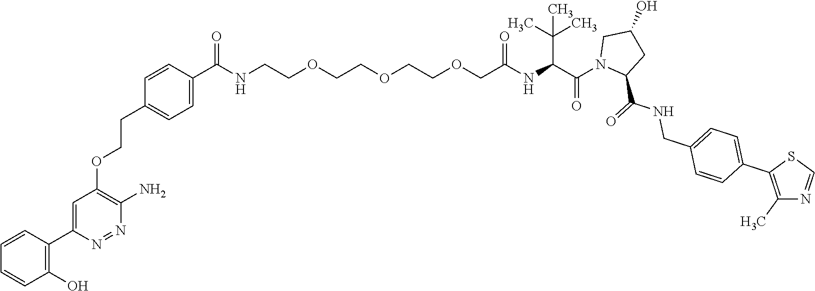 [SMILES: Cc1ncsc1-c1ccc(CNC(=O)[C@@H]2C[C@@H](O)CN2C(=O)[C@@H](NC(=O)COCCOCCOCCNC(=O)c2ccc(CCOc3cc(-c4ccccc4O)nnc3N)cc2)C(C)(C)C)cc1] |
| 61 | 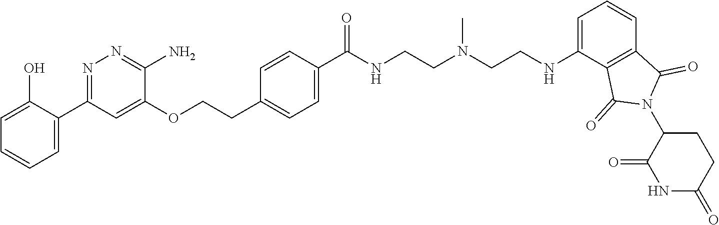 [SMILES: CN(CCNC(=O)c1ccc(CCOc2cc(-c3ccccc3O)nnc2N)cc1)CCNc1cccc2c1C(=O)N(C1CCC(=O)NC1=O)C2=O] |
| 62 | 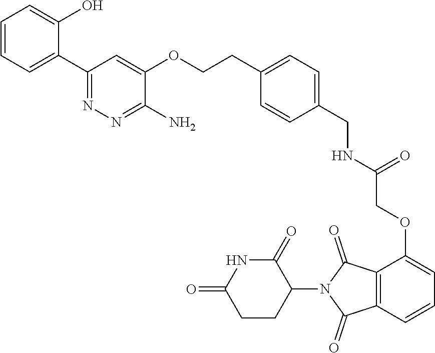 [SMILES: Nc1nnc(-c2ccccc2O)cc1OCCc1ccc(CNC(=O)COc2cccc3c2C(=O)N(C2CCC(=O)NC2=O)C3=O)cc1] |
| 63 | 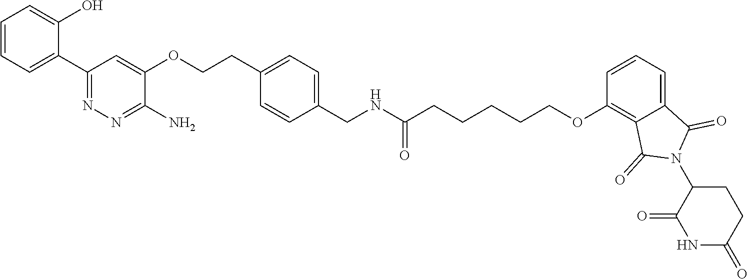 [SMILES: Nc1nnc(-c2ccccc2O)cc1OCCc1ccc(CNC(=O)CCCCCOc2cccc3c2C(=O)N(C2CCC(=O)NC2=O)C3=O)cc1] |
| 64 | 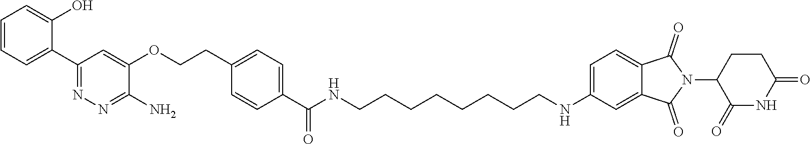 [SMILES: Nc1nnc(-c2ccccc2O)cc1OCCc1ccc(C(=O)NCCCCCCCCNc2ccc3c(c2)C(=O)N(C2CCC(=O)NC2=O)C3=O)cc1] |
| 65 | 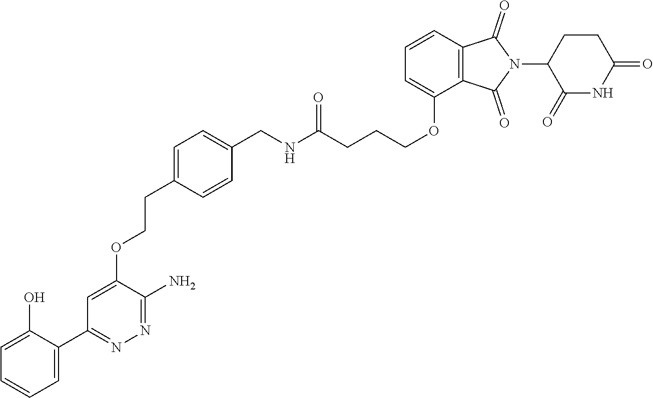 [SMILES: Nc1nnc(-c2ccccc2O)cc1OCCc1ccc(CNC(=O)CCCOc2cccc3c2C(=O)N(C2CCC(=O)NC2=O)C3=O)cc1] |
| 66 | 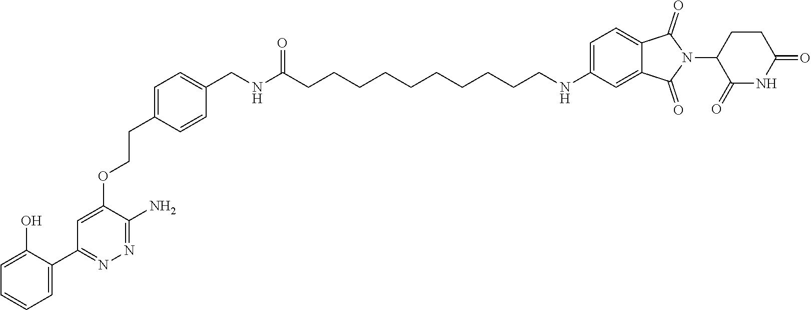 [SMILES: Nc1nnc(-c2ccccc2O)cc1OCCc1ccc(CNC(=O)CCCCCCCCCCNc2ccc3c(c2)C(=O)N(C2CCC(=O)NC2=O)C3=O)cc1] |
| 67 | 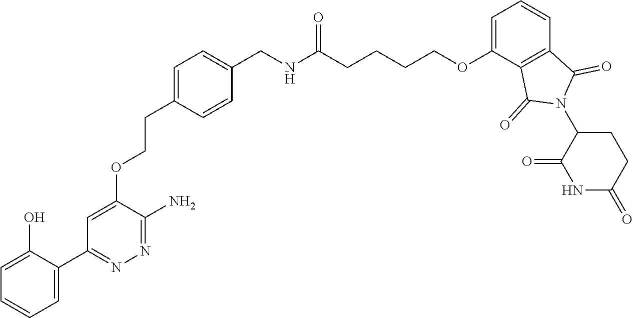 [SMILES: Nc1nnc(-c2ccccc2O)cc1OCCc1ccc(CNC(=O)CCCCOc2cccc3c2C(=O)N(C2CCC(=O)NC2=O)C3=O)cc1] |
| 68 | 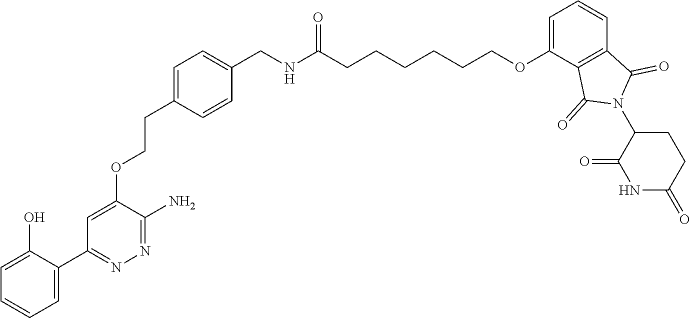 [SMILES: Nc1nnc(-c2ccccc2O)cc1OCCc1ccc(CNC(=O)CCCCCCOc2cccc3c2C(=O)N(C2CCC(=O)NC2=O)C3=O)cc1] |
| 69 | 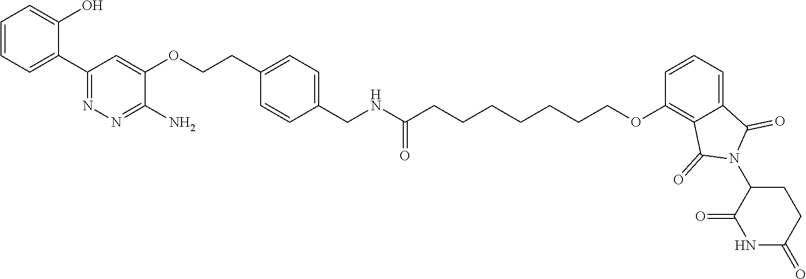 [SMILES: Nc1nnc(-c2ccccc2O)cc1OCCc1ccc(CNC(=O)CCCCCCCOc2cccc3c2C(=O)N(C2CCC(=O)NC2=O)C3=O)cc1] |
| 70 | 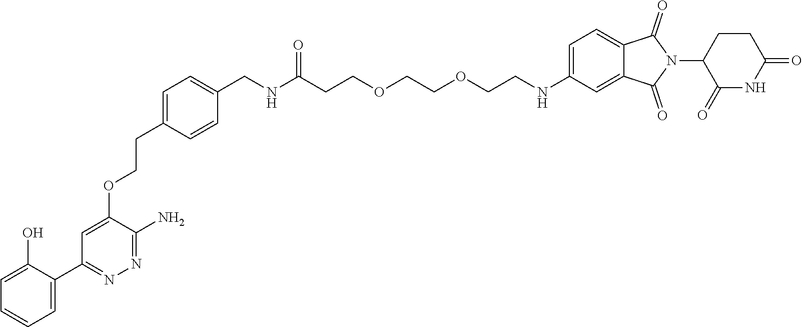 [SMILES: Nc1nnc(-c2ccccc2O)cc1OCCc1ccc(CNC(=O)CCOCCOCCNc2ccc3c(c2)C(=O)N(C2CCC(=O)NC2=O)C3=O)cc1] |
| 71 | 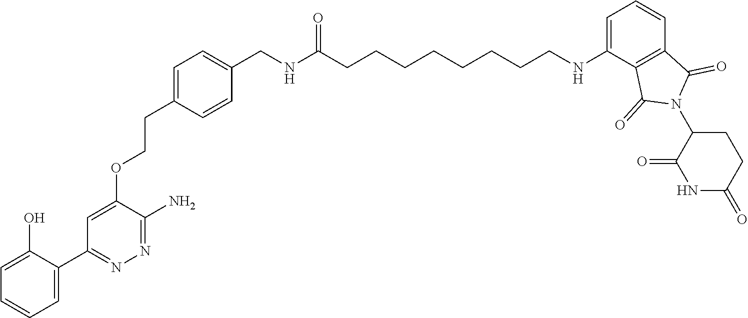 [SMILES: Nc1nnc(-c2ccccc2O)cc1OCCc1ccc(CNC(=O)CCCCCCCCNc2cccc3c2C(=O)N(C2CCC(=O)NC2=O)C3=O)cc1] |
| 72 | 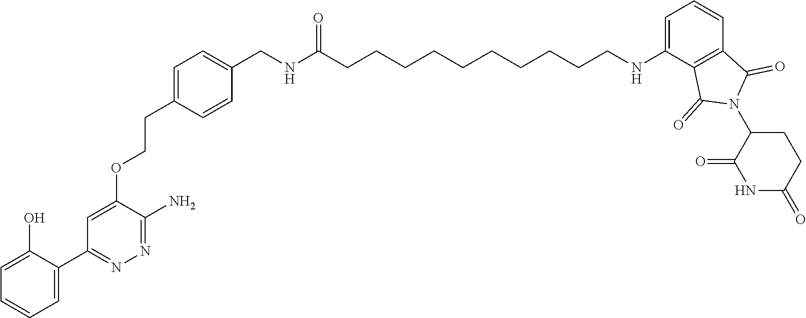 [SMILES: Nc1nnc(-c2ccccc2O)cc1OCCc1ccc(CNC(=O)CCCCCCCCCCNc2cccc3c2C(=O)N(C2CCC(=O)NC2=O)C3=O)cc1] |
| 73 | 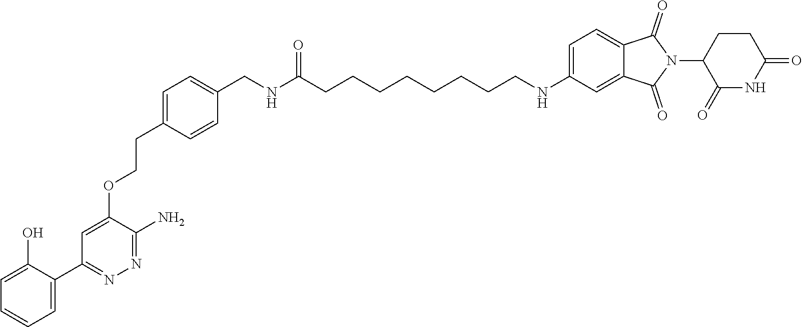 [SMILES: Nc1nnc(-c2ccccc2O)cc1OCCc1ccc(CNC(=O)CCCCCCCCNc2ccc3c(c2)C(=O)N(C2CCC(=O)NC2=O)C3=O)cc1] |
| 74 | 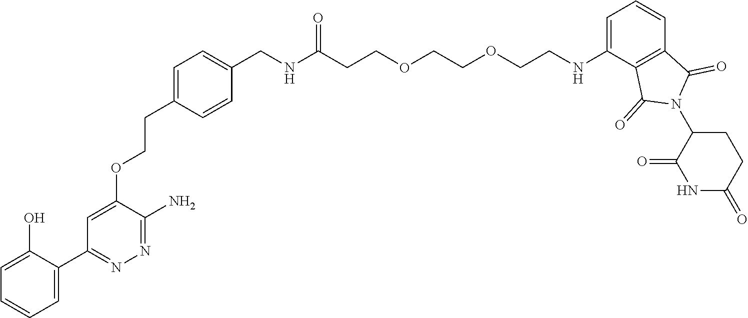 [SMILES: Nc1nnc(-c2ccccc2O)cc1OCCc1ccc(CNC(=O)CCOCCOCCNc2cccc3c2C(=O)N(C2CCC(=O)NC2=O)C3=O)cc1] |
| 75 | 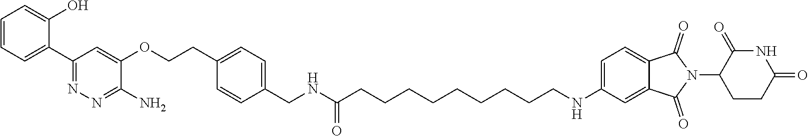 [SMILES: Nc1nnc(-c2ccccc2O)cc1OCCc1ccc(CNC(=O)CCCCCCCCCNc2ccc3c(c2)C(=O)N(C2CCC(=O)NC2=O)C3=O)cc1] |

TABLE 2
Compounds of the Invention

| Comp. | |
|---|---|
| No. | Structure |
| 76 | 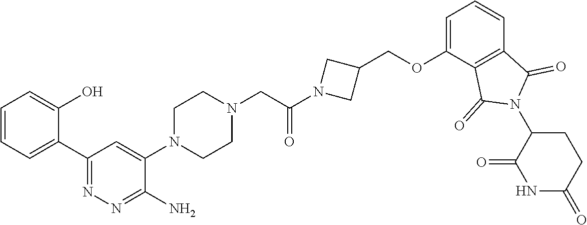 [SMILES: Nc1nnc(-c2ccccc2O)cc1N1CCN(CC(=O)N2CC(COc3cccc4c3C(=O)N(C3CCC(=O)NC3=O)C4=O)C2)CC1] |
| 77 | 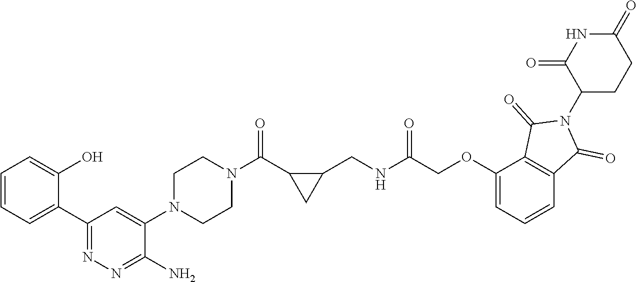 [SMILES: Nc1nnc(-c2ccccc2O)cc1N1CCN(C(=O)C2CC2CNC(=O)COc2cccc3c2C(=O)N(C2CCC(=O)NC2=O)C3=O)CC1] |
| 78 | 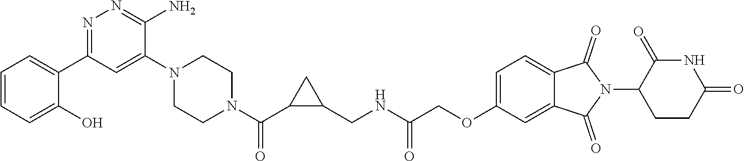 [SMILES: Nc1nnc(-c2ccccc2O)cc1N1CCN(C(=O)C2CC2CNC(=O)COc2ccc3c(c2)C(=O)N(C2CCC(=O)NC2=O)C3=O)CC1] |
| 79 | 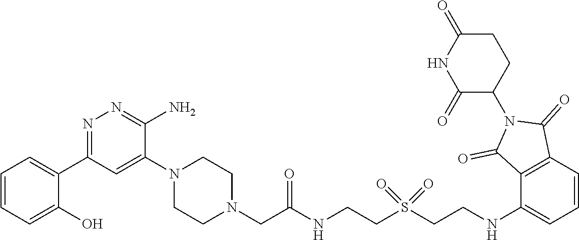 [SMILES: Nc1nnc(-c2ccccc2O)cc1N1CCN(CC(=O)NCCS(=O)(=O)CCNc2cccc3c2C(=O)N(C2CCC(=O)NC2=O)C3=O)CC1] |
| 80 | 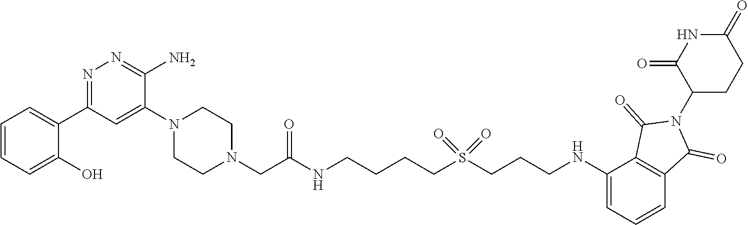 [SMILES: Nc1nnc(-c2ccccc2O)cc1N1CCN(CC(=O)NCCCCS(=O)(=O)CCCNc2cccc3c2C(=O)N(C2CCC(=O)NC2=O)C3=O)CC1] |
| 81 | 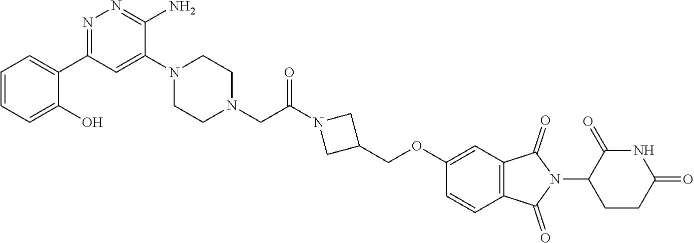 [SMILES: Nc1nnc(-c2ccccc2O)cc1N1CCN(CC(=O)N2CC(COc3ccc4c(c3)C(=O)N(C3CCC(=O)NC3=O)C4=O)C2)CC1] |
| 82 | 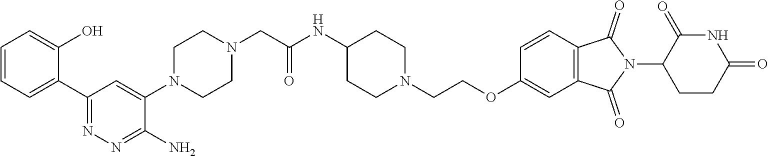 [SMILES: Nc1nnc(-c2ccccc2O)cc1N1CCN(CC(=O)NC2CCN(CCOc3ccc4c(c3)C(=O)N(C3CCC(=O)NC3=O)C4=O)CC2)CC1] |
| 83 | 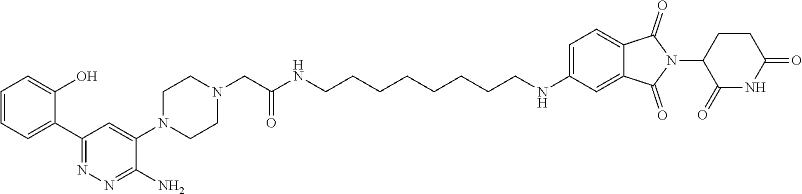 [SMILES: Nc1nnc(-c2ccccc2O)cc1N1CCN(CC(=O)NCCCCCCCCNc2ccc3c(c2)C(=O)N(C2CCC(=O)NC2=O)C3=O)CC1] |
| 84 | 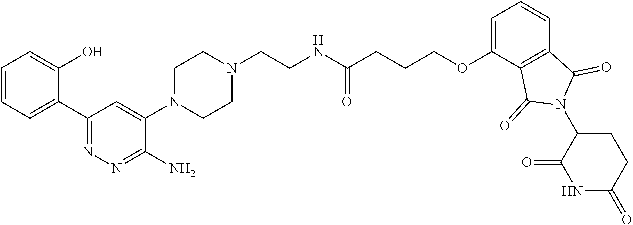 [SMILES: Nc1nnc(-c2ccccc2O)cc1N1CCN(CCNC(=O)CCCOc2cccc3c2C(=O)N(C2CCC(=O)NC2=O)C3=O)CC1] |
| 85 | 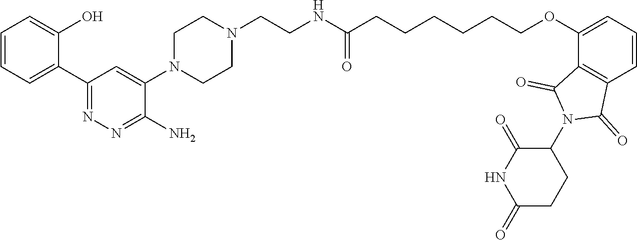 [SMILES: Nc1nnc(-c2ccccc2O)cc1N1CCN(CCNC(=O)CCCCCCOc2cccc3c2C(=O)N(C2CCC(=O)NC2=O)C3=O)CC1] |
| 86 | 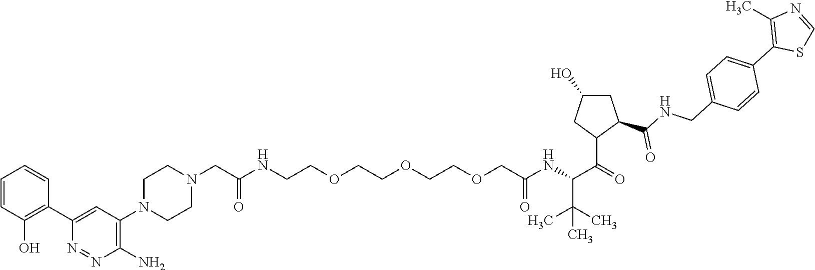 [SMILES: Cc1ncsc1-c1ccc(CNC(=O)[C@@H]2C[C@@H](O)CC2C(=O)[C@@H](NC(=O)COCCOCCOCCNC(=O)CN2CCN(c3cc(-c4ccccc4O)nnc3N)CC2)C(C)(C)C)cc1] |
| 87 | 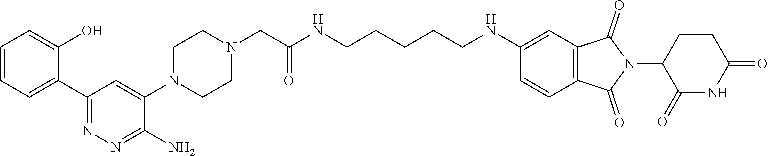 [SMILES: Nc1nnc(-c2ccccc2O)cc1N1CCN(CC(=O)NCCCCCNc2ccc3c(c2)C(=O)N(C2CCC(=O)NC2=O)C3=O)CC1] |
| 88 | 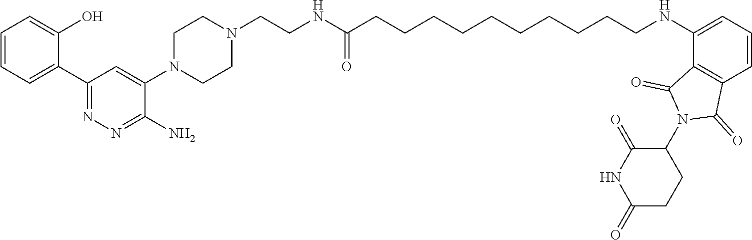 [SMILES: Nc1nnc(-c2ccccc2O)cc1N1CCN(CCNC(=O)CCCCCCCCCCNc2cccc3c2C(=O)N(C2CCC(=O)NC2=O)C3=O)CC1] |
| 89 | 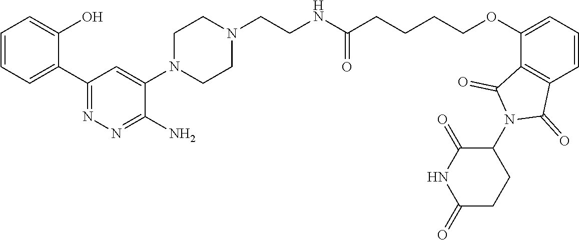 [SMILES: Nc1nnc(-c2ccccc2O)cc1N1CCN(CCNC(=O)CCCCOc2cccc3c2C(=O)N(C2CCC(=O)NC2=O)C3=O)CC1] |
| 90 | 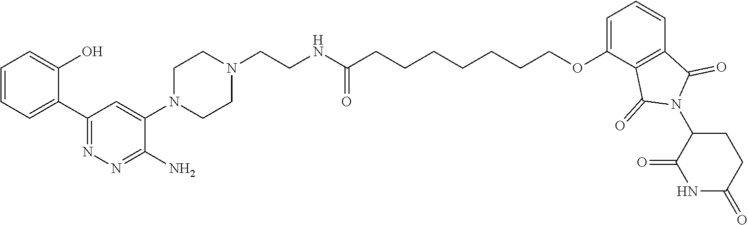 [SMILES: Nc1nnc(-c2ccccc2O)cc1N1CCN(CCNC(=O)CCCCCCCOc2cccc3c2C(=O)N(C2CCC(=O)NC2=O)C3=O)CC1] |
| 91 | 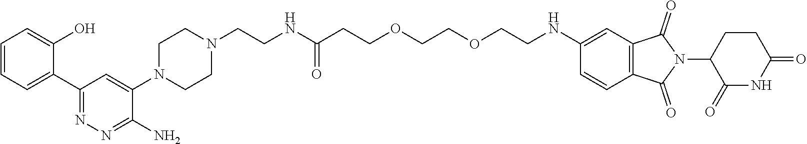 [SMILES: Nc1nnc(-c2ccccc2O)cc1N1CCN(CCNC(=O)CCOCCOCCNc2ccc3c(c2)C(=O)N(C2CCC(=O)NC2=O)C3=O)CC1] |
| 92 | 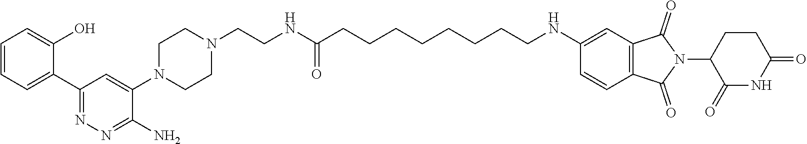 [SMILES: Nc1nnc(-c2ccccc2O)cc1N1CCN(CCNC(=O)CCCCCCCCNc2ccc3c(c2)C(=O)N(C2CCC(=O)NC2=O)C3=O)CC1] |
| 93 | 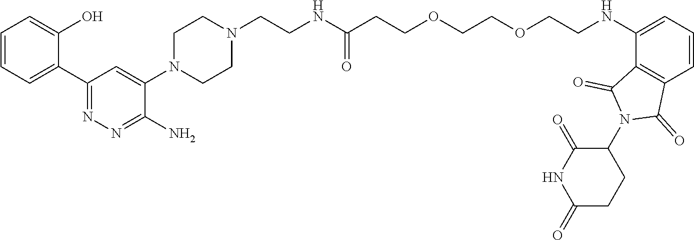 [SMILES: Nc1nnc(-c2ccccc2O)cc1N1CCN(CCNC(=O)CCOCCOCCNc2cccc3c2C(=O)N(C2CCC(=O)NC2=O)C3=O)CC1] |
| 94 | 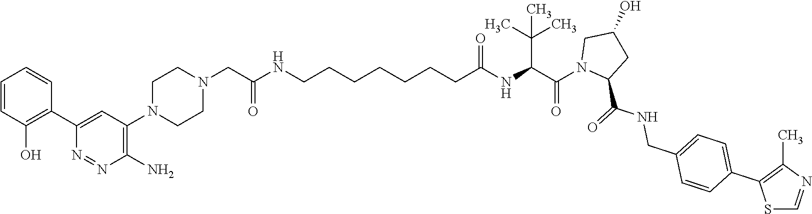 [SMILES: Cc1ncsc1-c1ccc(CNC(=O)[C@@H]2C[C@@H](O)CN2C(=O)[C@@H](NC(=O)CCCCCCCNC(=O)CN2CCN(c3cc(-c4ccccc4O)nnc3N)CC2)C(C)(C)C)cc1] |
| 95 | 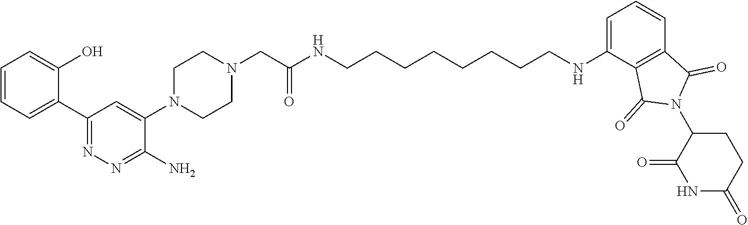 [SMILES: Nc1nnc(-c2ccccc2O)cc1N1CCN(CC(=O)NCCCCCCCCNc2cccc3c2C(=O)N(C2CCC(=O)NC2=O)C3=O)CC1] |
| 96 | 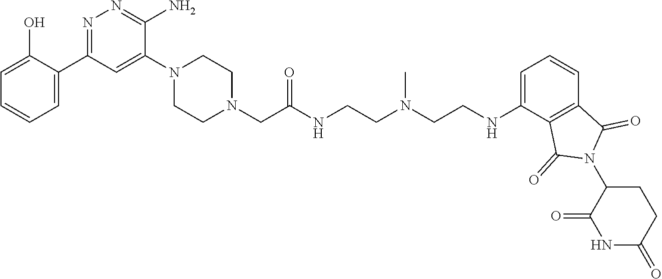 [SMILES: CN(CCNC(=O)CN1CCN(c2cc(-c3ccccc3O)nnc2N)CC1)CCNc1cccc2c1C(=O)N(C1CCC(=O)NC1=O)C2=O] |
| 97 | 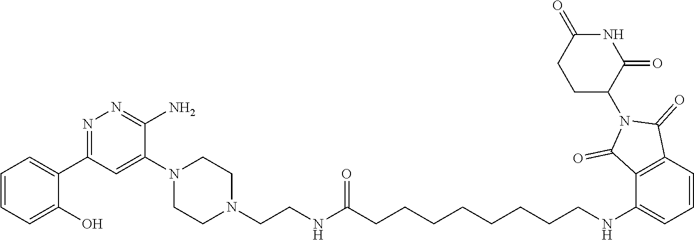 [SMILES: Nc1nnc(-c2ccccc2O)cc1N1CCN(CCNC(=O)CCCCCCCCNc2cccc3c2C(=O)N(C2CCC(=O)NC2=O)C3=O)CC1] |
| 98 | 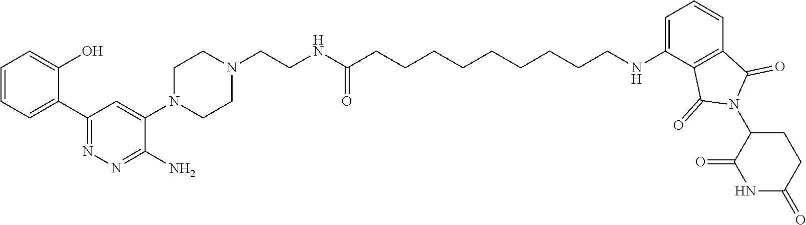 [SMILES: Nc1nnc(-c2ccccc2O)cc1N1CCN(CCNC(=O)CCCCCCCCCNc2cccc3c2C(=O)N(C2CCC(=O)NC2=O)C3=O)CC1] |
| 99 | 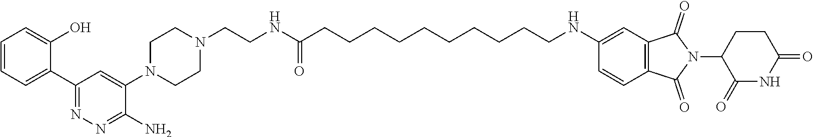 [SMILES: Nc1nnc(-c2ccccc2O)cc1N1CCN(CCNC(=O)CCCCCCCCCCNc2ccc3c(c2)C(=O)N(C2CCC(=O)NC2=O)C3=O)CC1] |
| 100 | 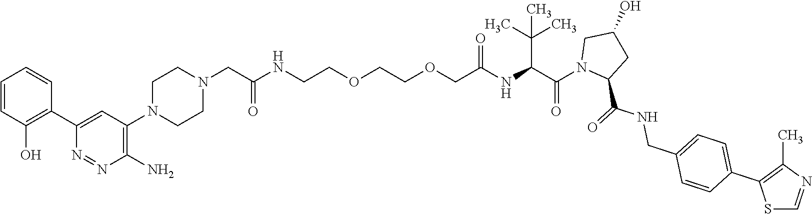 [SMILES: Cc1ncsc1-c1ccc(CNC(=O)[C@@H]2C[C@@H](O)CN2C(=O)[C@@H](NC(=O)COCCOCCNC(=O)CN2CCN(c3cc(-c4ccccc4O)nnc3N)CC2)C(C)(C)C)cc1] |
| 101 | 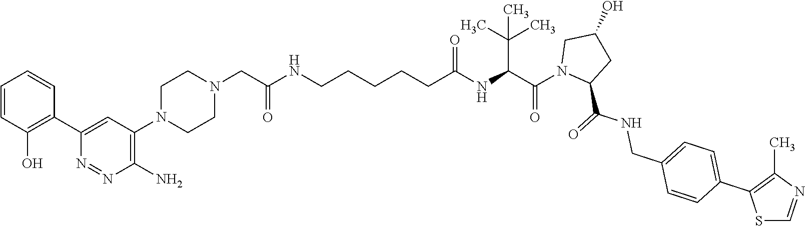 [SMILES: Cc1ncsc1-c1ccc(CNC(=O)[C@@H]2C[C@@H](O)CN2C(=O)[C@@H](NC(=O)CCCCCNC(=O)CN2CCN(c3cc(-c4ccccc4O)nnc3N)CC2)C(C)(C)C)cc1] |
| 102 | 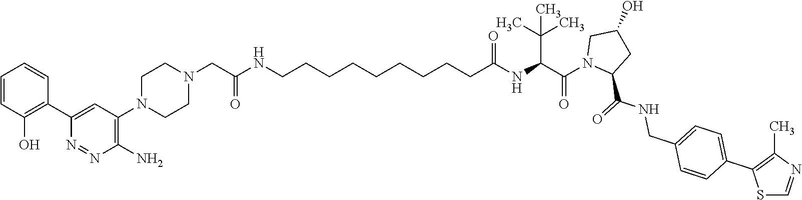 [SMILES: Cc1ncsc1-c1ccc(CNC(=O)[C@@H]2C[C@@H](O)CN2C(=O)[C@@H](NC(=O)CCCCCCCCCNC(=O)CN2CCN(c3cc(-c4ccccc4O)nnc3N)CC2)C(C)(C)C)cc1] |
| 103 | 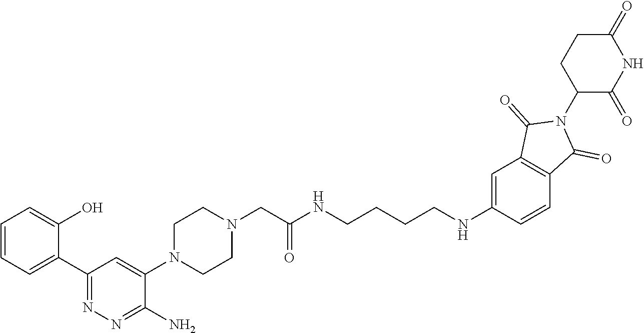 [SMILES: Nc1nnc(-c2ccccc2O)cc1N1CCN(CC(=O)NCCCCNc2ccc3c(c2)C(=O)N(C2CCC(=O)NC2=O)C3=O)CC1] |
| 104 | 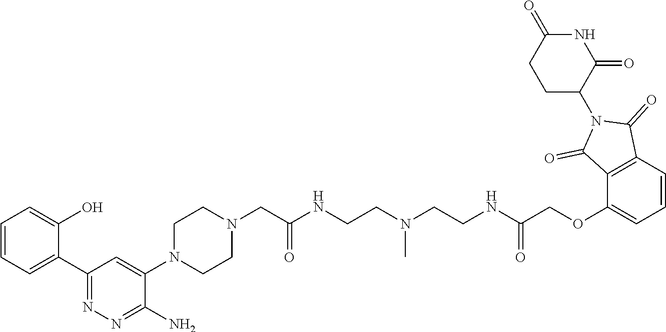 [SMILES: CN(CCNC(=O)COc1cccc2c1C(=O)N(C1CCC(=O)NC1=O)C2=O)CCNC(=O)CN1CCN(c2cc(-c3ccccc3O)nnc2N)CC1] |
| 105 | 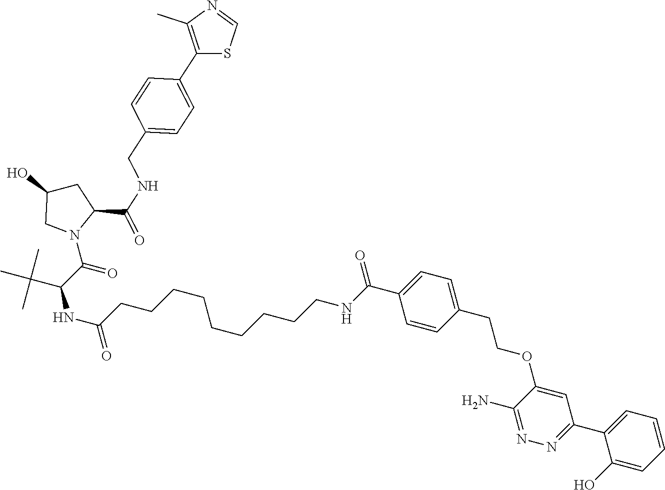 [SMILES: Cc1ncsc1-c1ccc(CNC(=O)[C@@H]2C[C@H](O)CN2C(=O)[C@@H](NC(=O)CCCCCCCCCNC(=O)c2ccc(CCOc3cc(-c4ccccc4O)nnc3N)cc2)C(C)(C)C)cc1] |
| 106 | 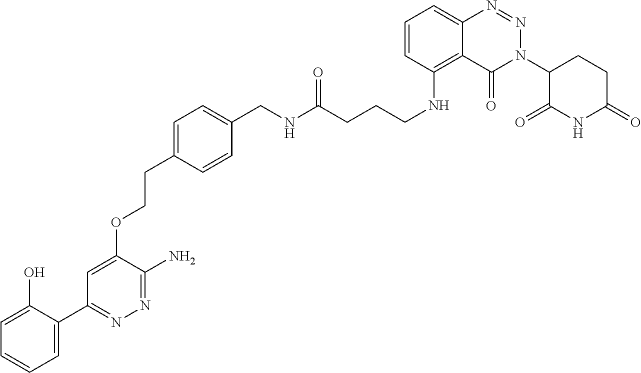 [SMILES: Nc1nnc(-c2ccccc2O)cc1OCCc1ccc(CNC(=O)CCCNc2cccc3nnn(C4CCC(=O)NC4=O)c(=O)c23)cc1] |
| 107 | 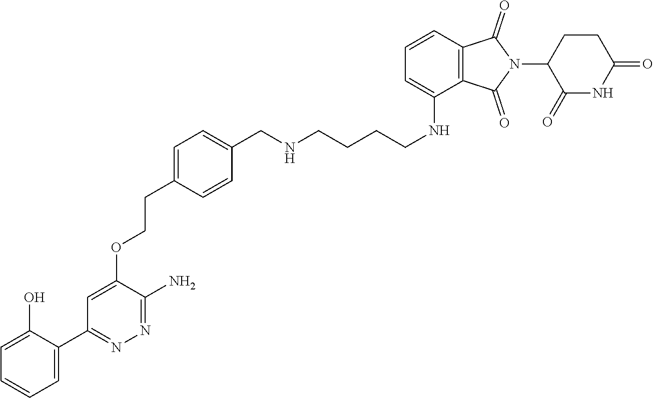 [SMILES: Nc1nnc(-c2ccccc2O)cc1OCCc1ccc(CNCCCCNc2cccc3c2C(=O)N(C2CCC(=O)NC2=O)C3=O)cc1] |
| 108 | 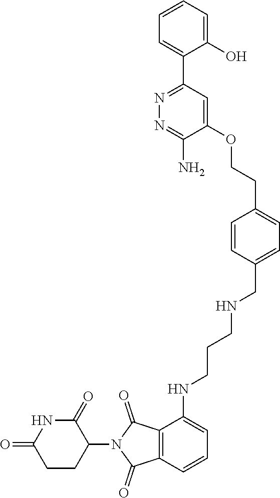 [SMILES: Nc1nnc(-c2ccccc2O)cc1OCCc1ccc(CNCCCNc2cccc3c2C(=O)N(C2CCC(=O)NC2=O)C3=O)cc1] |
| 109 | 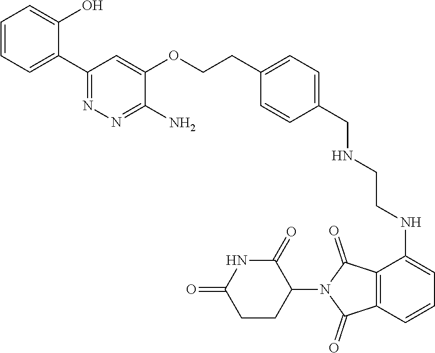 [SMILES: Nc1nnc(-c2ccccc2O)cc1OCCc1ccc(CNCCNc2cccc3c2C(=O)N(C2CCC(=O)NC2=O)C3=O)cc1] |
| 110 | 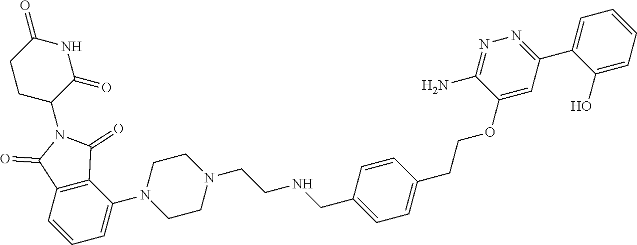 [SMILES: Nc1nnc(-c2ccccc2O)cc1OCCc1ccc(CNCCN2CCN(c3cccc4c3C(=O)N(C3CCC(=O)NC3=O)C4=O)CC2)cc1] |
| 111 | 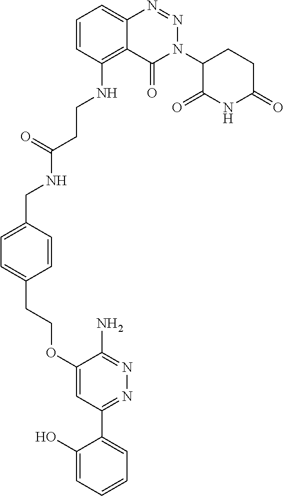 [SMILES: Nc1nnc(-c2ccccc2O)cc1OCCc1ccc(CNC(=O)CCNc2cccc3nnn(C4CCC(=O)NC4=O)c(=O)c23)cc1] |
| 112 | 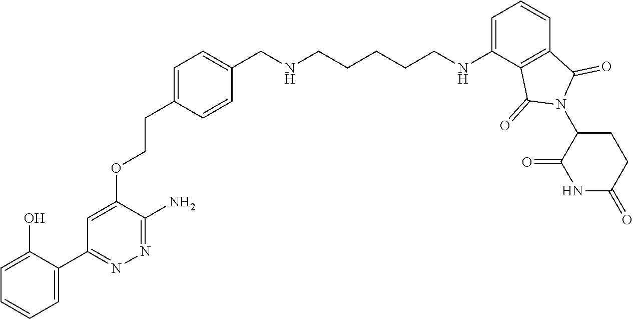 [SMILES: Nc1nnc(-c2ccccc2O)cc1OCCc1ccc(CNCCCCCNc2cccc3c2C(=O)N(C2CCC(=O)NC2=O)C3=O)cc1] |
| 113 | 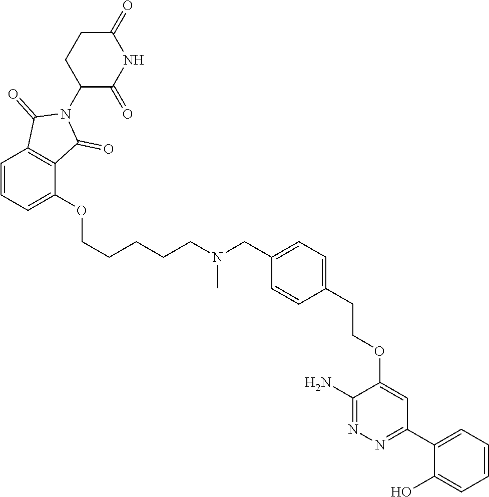 [SMILES: CN(CCCCCOc1cccc2c1C(=O)N(C1CCC(=O)NC1=O)C2=O)Cc1ccc(CCOc2cc(-c3ccccc3O)nnc2N)cc1] |
| 114 | 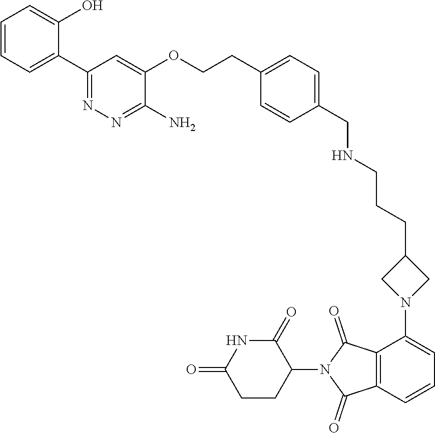 [SMILES: Nc1nnc(-c2ccccc2O)cc1OCCc1ccc(CNCCCC2CN(c3cccc4c3C(=O)N(C3CCC(=O)NC3=O)C4=O)C2)cc1] |
| 115 | 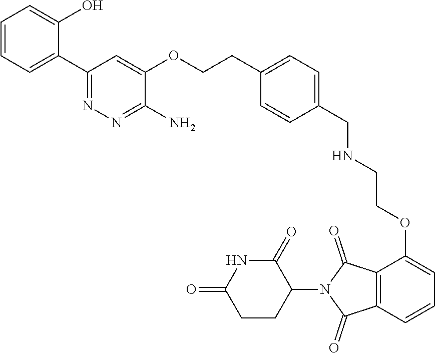 [SMILES: Nc1nnc(-c2ccccc2O)cc1OCCc1ccc(CNCCOc2cccc3c2C(=O)N(C2CCC(=O)NC2=O)C3=O)cc1] |
| 116 | 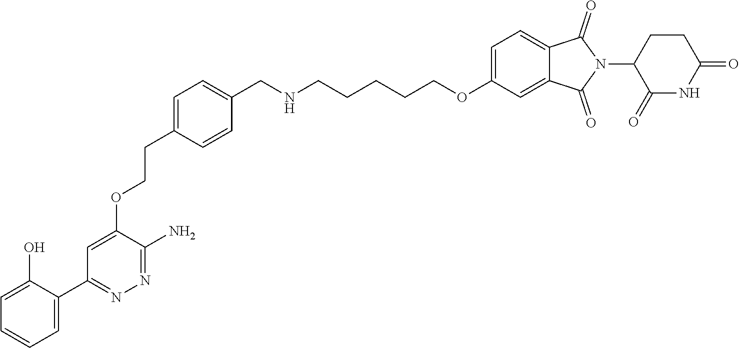 [SMILES: Nc1nnc(-c2ccccc2O)cc1OCCc1ccc(CNCCCCCOc2ccc3c(c2)C(=O)N(C2CCC(=O)NC2=O)C3=O)cc1] |
| 117 | 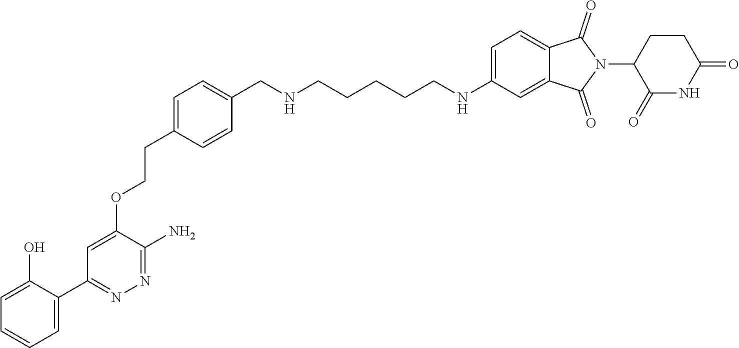 [SMILES: Nc1nnc(-c2ccccc2O)cc1OCCc1ccc(CNCCCCCNc2ccc3c(c2)C(=O)N(C2CCC(=O)NC2=O)C3=O)cc1] |
| 118 | 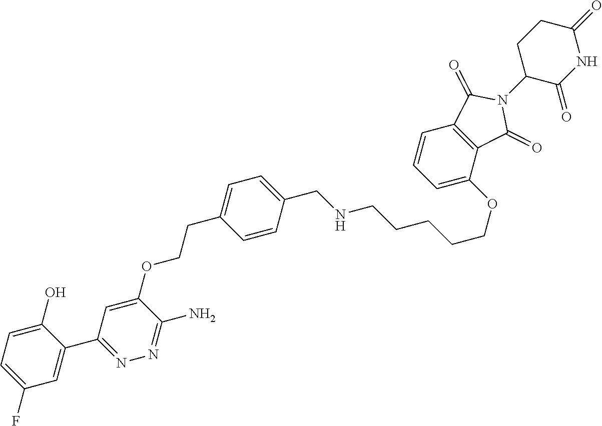 [SMILES: Nc1nnc(-c2cc(F)ccc2O)cc1OCCc1ccc(CNCCCCCOc2cccc3c2C(=O)N(C2CCC(=O)NC2=O)C3=O)cc1] |
| 119 | 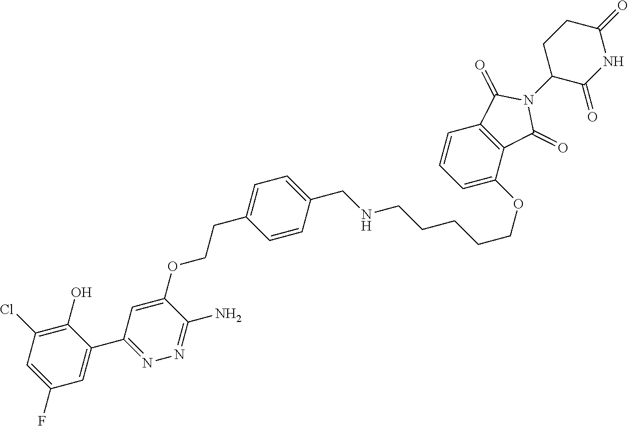 [SMILES: Nc1nnc(-c2cc(F)cc(Cl)c2O)cc1OCCc1ccc(CNCCCCCOc2cccc3c2C(=O)N(C2CCC(=O)NC2=O)C3=O)cc1] |
| 120 | 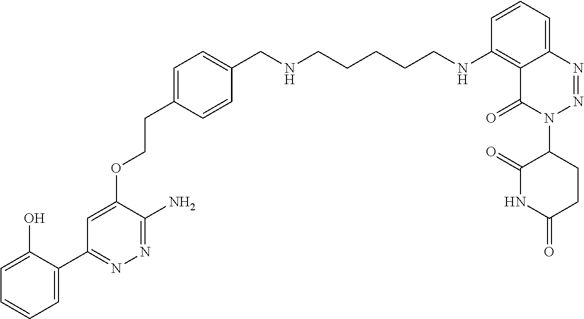 [SMILES: Nc1nnc(-c2ccccc2O)cc1OCCc1ccc(CNCCCCCNc2cccc3nnn(C4CCC(=O)NC4=O)c(=O)c23)cc1] |
| 121 | 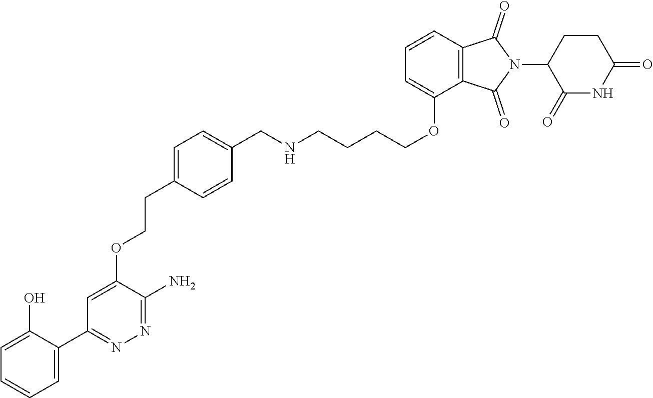 [SMILES: Nc1nnc(-c2ccccc2O)cc1OCCc1ccc(CNCCCCOc2cccc3c2C(=O)N(C2CCC(=O)NC2=O)C3=O)cc1] |
| 122 | 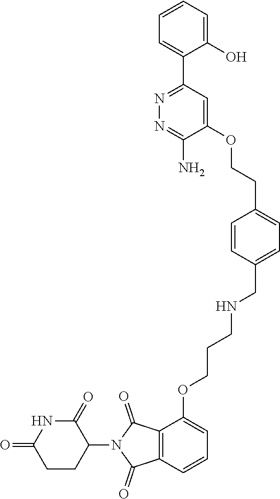 [SMILES: Nc1nnc(-c2ccccc2O)cc1OCCc1ccc(CNCCCOc2cccc3c2C(=O)N(C2CCC(=O)NC2=O)C3=O)cc1] |
| 123 | 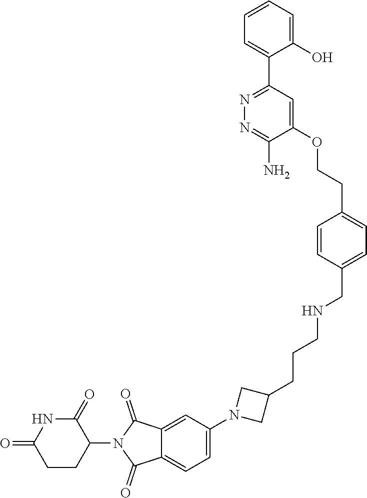 [SMILES: Nc1nnc(-c2ccccc2O)cc1OCCc1ccc(CNCCCC2CN(c3ccc4c(c3)C(=O)N(C3CCC(=O)NC3=O)C4=O)C2)cc1] |
| 124 | 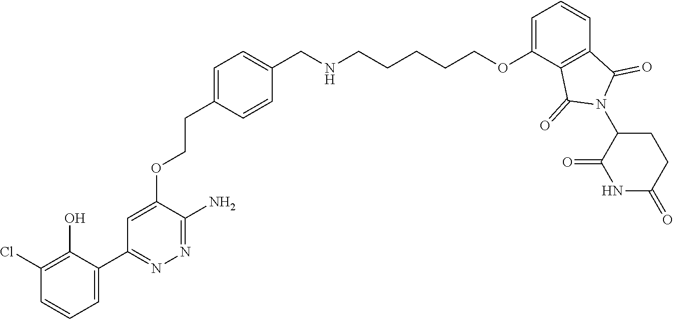 [SMILES: Nc1nnc(-c2cccc(Cl)c2O)cc1OCCc1ccc(CNCCCCCOc2cccc3c2C(=O)N(C2CCC(=O)NC2=O)C3=O)cc1] |
| 125 | 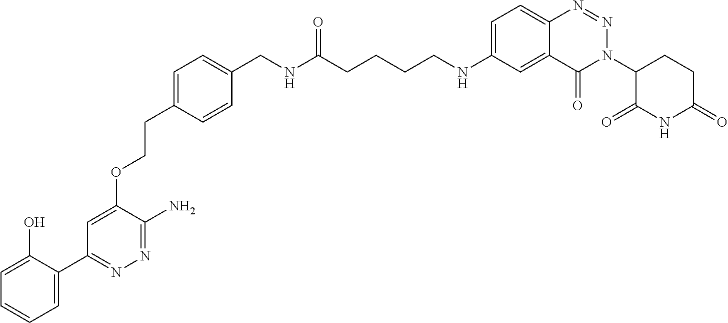 [SMILES: Nc1nnc(-c2ccccc2O)cc1OCCc1ccc(CNC(=O)CCCCNc2ccc3nnn(C4CCC(=O)NC4=O)c(=O)c3c2)cc1] |
| 126 | 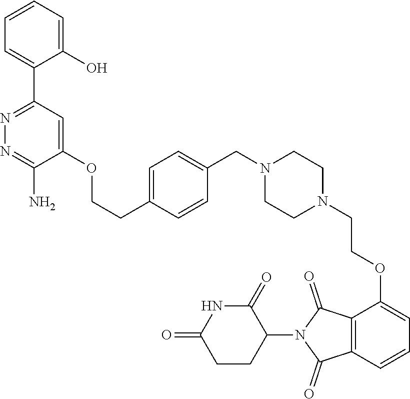 [SMILES: Nc1nnc(-c2ccccc2O)cc1OCCc1ccc(CN2CCN(CCOc3cccc4c3C(=O)N(C3CCC(=O)NC3=O)C4=O)CC2)cc1] |
| 127 | 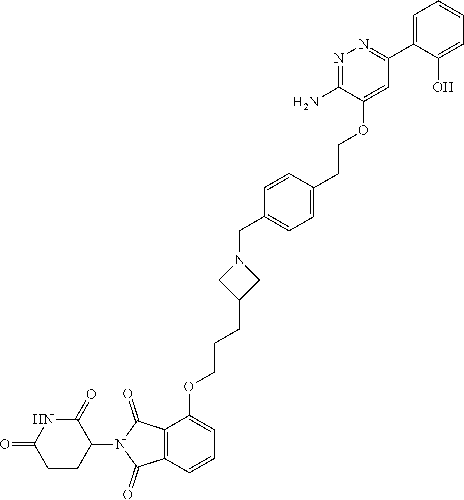 [SMILES: Nc1nnc(-c2ccccc2O)cc1OCCc1ccc(CN2CC(CCCOc3cccc4c3C(=O)N(C3CCC(=O)NC3=O)C4=O)C2)cc1] |
| 128 | 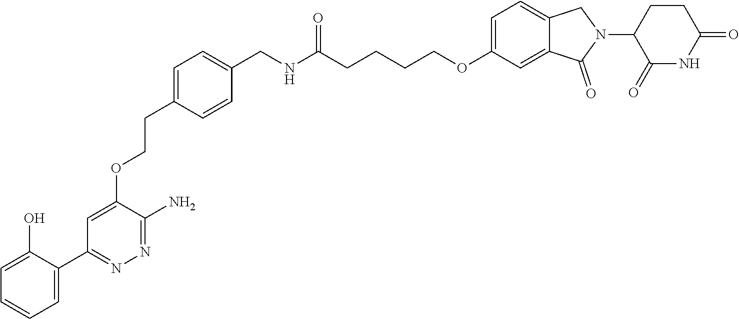 [SMILES: Nc1nnc(-c2ccccc2O)cc1OCCc1ccc(CNC(=O)CCCCOc2ccc3c(c2)C(=O)N(C2CCC(=O)NC2=O)C3)cc1] |
| 129 | 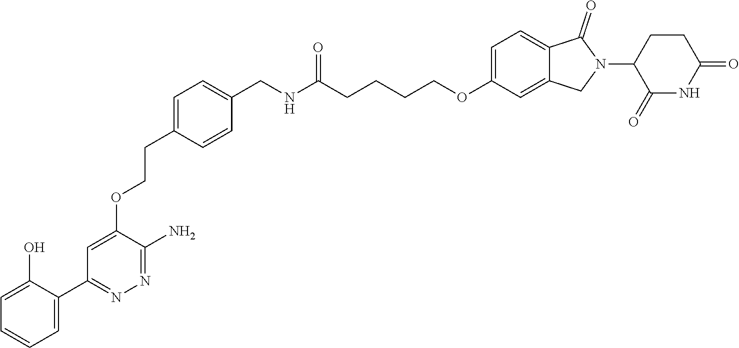 [SMILES: Nc1nnc(-c2ccccc2O)cc1OCCc1ccc(CNC(=O)CCCCOc2ccc3c(c2)CN(C2CCC(=O)NC2=O)C3=O)cc1] |
| 130 | 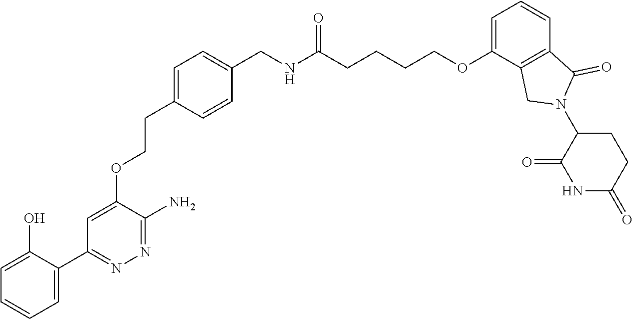 [SMILES: Nc1nnc(-c2ccccc2O)cc1OCCc1ccc(CNC(=O)CCCCOc2cccc3c2CN(C2CCC(=O)NC2=O)C3=O)cc1] |
| 131 | 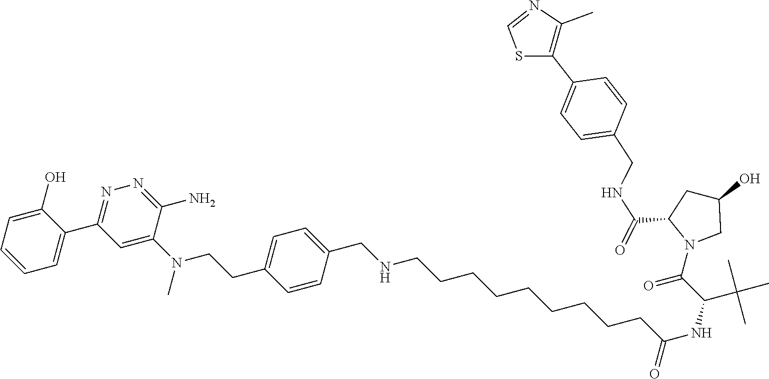 [SMILES: Cc1ncsc1-c1ccc(CNC(=O)[C@@H]2C[C@@H](O)CN2C(=O)[C@@H](NC(=O)CCCCCCCCCNCc2ccc(CCN(C)c3cc(-c4ccccc4O)nnc3N)cc2)C(C)(C)C)cc1] |
| 132 | 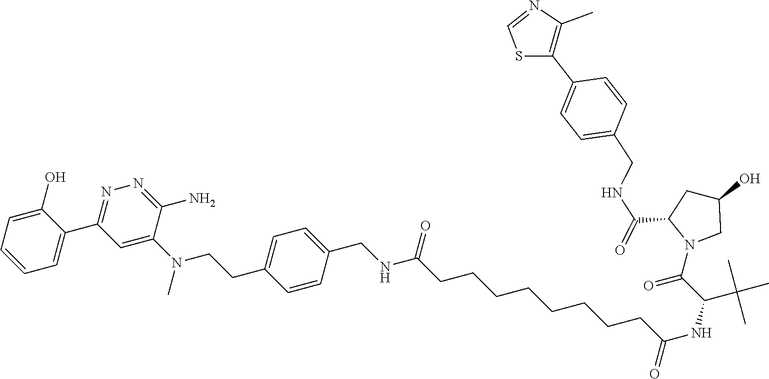 [SMILES: Cc1ncsc1-c1ccc(CNC(=O)[C@@H]2C[C@@H](O)CN2C(=O)[C@@H](NC(=O)CCCCCCCCC(=O)NCc2ccc(CCN(C)c3cc(-c4ccccc4O)nnc3N)cc2)C(C)(C)C)cc1] |
| 133 | 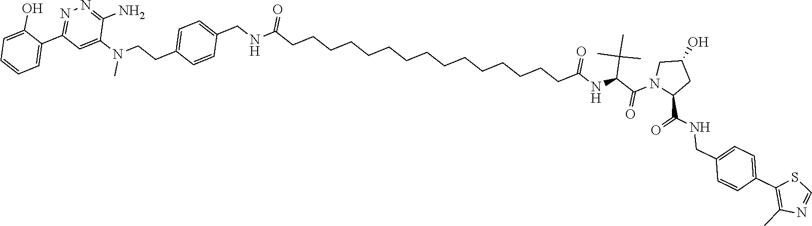 [SMILES: Cc1ncsc1-c1ccc(CNC(=O)[C@@H]2C[C@@H](O)CN2C(=O)[C@@H](NC(=O)CCCCCCCCCCCCCCCC(=O)NCc2ccc(CCN(C)c3cc(-c4ccccc4O)nnc3N)cc2)C(C)(C)C)cc1] |
| 134 | 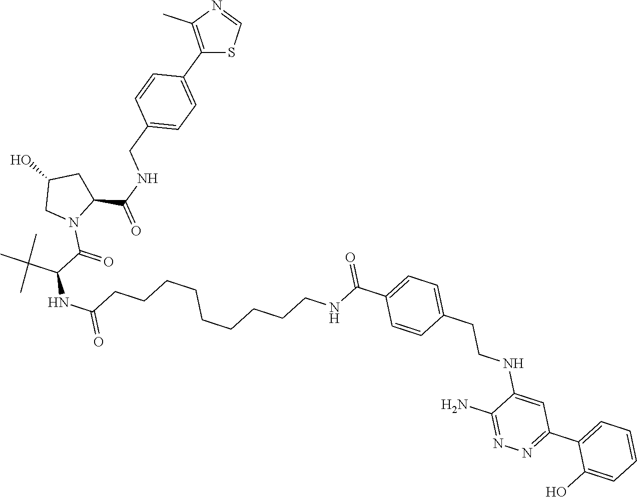 [SMILES: Cc1ncsc1-c1ccc(CNC(=O)[C@@H]2C[C@@H](O)CN2C(=O)[C@@H](NC(=O)CCCCCCCCCNC(=O)c2ccc(CCNc3cc(-c4ccccc4O)nnc3N)cc2)C(C)(C)C)cc1] |
| 135 | 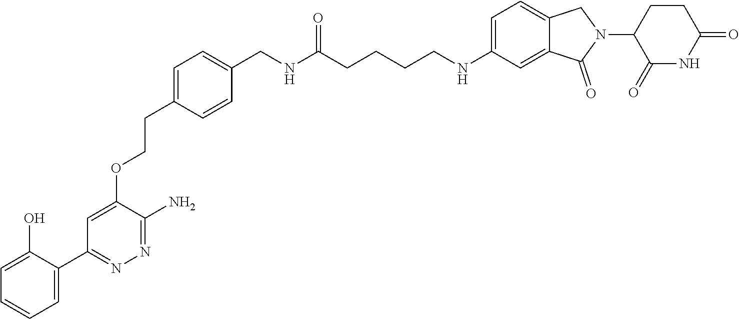 [SMILES: Nc1nnc(-c2ccccc2O)cc1OCCc1ccc(CNC(=O)CCCCNc2ccc3c(c2)C(=O)N(C2CCC(=O)NC2=O)C3)cc1] |
| 136 | 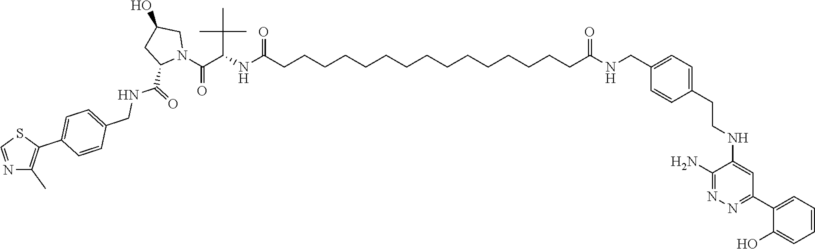 [SMILES: Cc1ncsc1-c1ccc(CNC(=O)[C@@H]2C[C@@H](O)CN2C(=O)[C@@H](NC(=O)CCCCCCCCCCCCCCCC(=O)NCc2ccc(CCNc3cc(-c4ccccc4O)nnc3N)cc2)C(C)(C)C)cc1] |
| 137 | 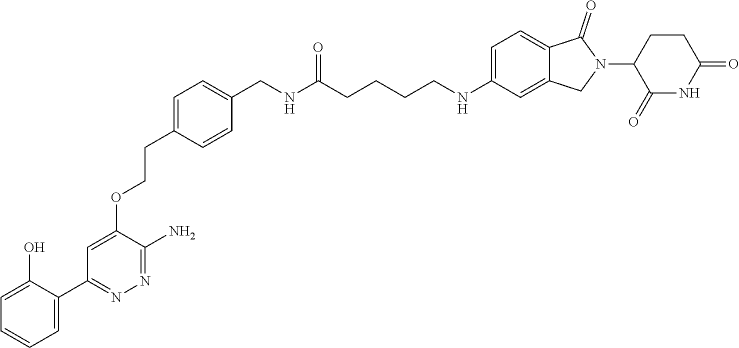 [SMILES: Nc1nnc(-c2ccccc2O)cc1OCCc1ccc(CNC(=O)CCCCNc2ccc3c(c2)CN(C2CCC(=O)NC2=O)C3=O)cc1] |
| 138 | 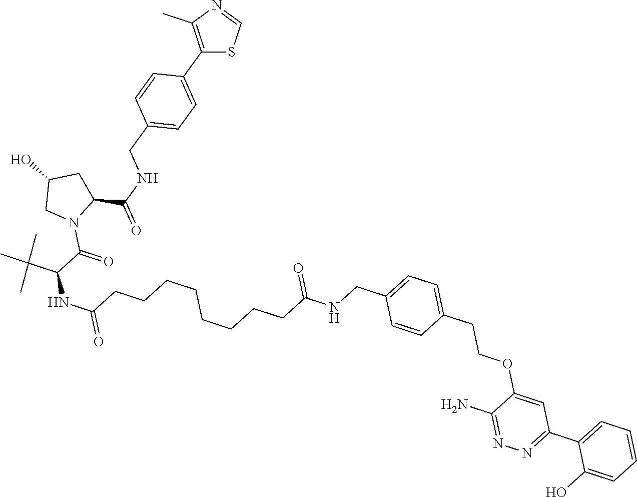 [SMILES: Cc1ncsc1-c1ccc(CNC(=O)[C@@H]2C[C@@H](O)CN2C(=O)[C@@H](NC(=O)CCCCCCCCC(=O)NCc2ccc(CCOc3cc(-c4ccccc4O)nnc3N)cc2)C(C)(C)C)cc1] |
| 139 | 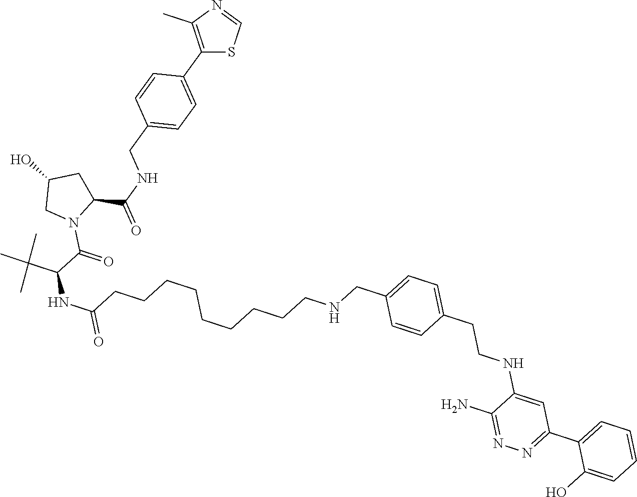 [SMILES: Cc1ncsc1-c1ccc(CNC(=O)[C@@H]2C[C@@H](O)CN2C(=O)[C@@H](NC(=O)CCCCCCCCCNCc2ccc(CCNc3cc(-c4ccccc4O)nnc3N)cc2)C(C)(C)C)cc1] |
| 140 | 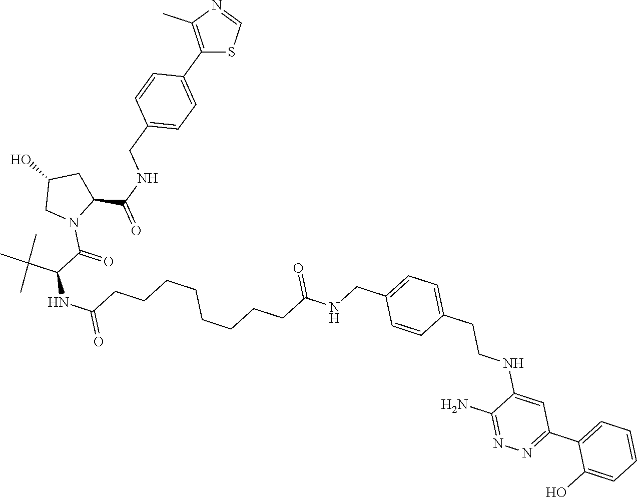 [SMILES: Cc1ncsc1-c1ccc(CNC(=O)[C@@H]2C[C@@H](O)CN2C(=O)[C@@H](NC(=O)CCCCCCCCC(=O)NCc2ccc(CCNc3cc(-c4ccccc4O)nnc3N)cc2)C(C)(C)C)cc1] |
| 141 | 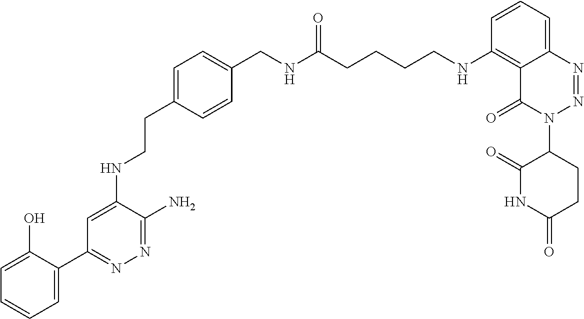 [SMILES: Nc1nnc(-c2ccccc2O)cc1NCCc1ccc(CNC(=O)CCCCNc2cccc3nnn(C4CCC(=O)NC4=O)c(=O)c23)cc1] |
| 142 | 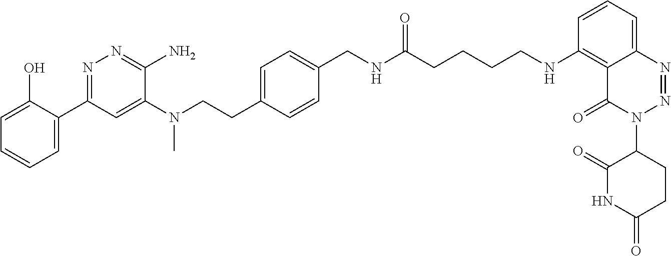 [SMILES: CN(CCc1ccc(CNC(=O)CCCCNc2cccc3nnn(C4CCC(=O)NC4=O)c(=O)c23)cc1)c1cc(-c2ccccc2O)nnc1N] |
| 143 | 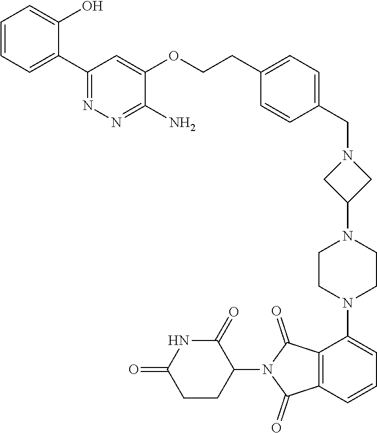 [SMILES: Nc1nnc(-c2ccccc2O)cc1OCCc1ccc(CN2CC(N3CCN(c4cccc5c4C(=O)N(C4CCC(=O)NC4=O)C5=O)CC3)C2)cc1] |
| 144 | 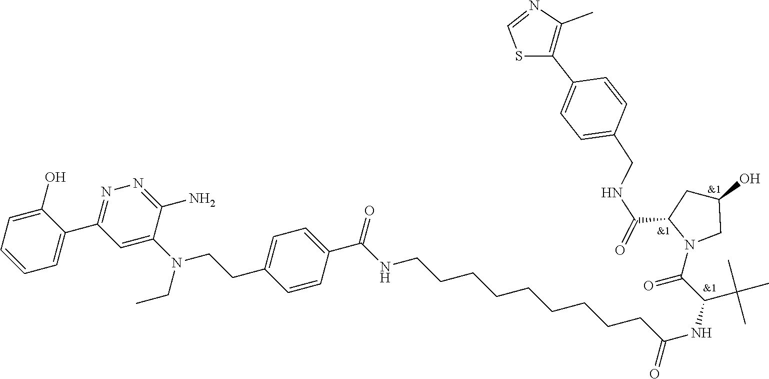 [SMILES: CCN(CCc1ccc(C(=O)NCCCCCCCCCC(=O)N[C@H](C(=O)N2C[C@H](O)C[C@H]2C(=O)NCc2ccc(-c3scnc3C)cc2)C(C)(C)C)cc1)c1cc(-c2ccccc2O)nnc1N] |
| 145 | 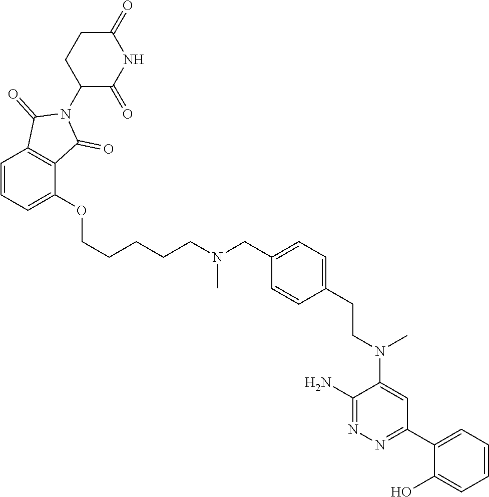 [SMILES: CN(CCCCCOc1cccc2c1C(=O)N(C1CCC(=O)NC1=O)C2=O)Cc1ccc(CCN(C)c2cc(-c3ccccc3O)nnc2N)cc1] |
| 146 | 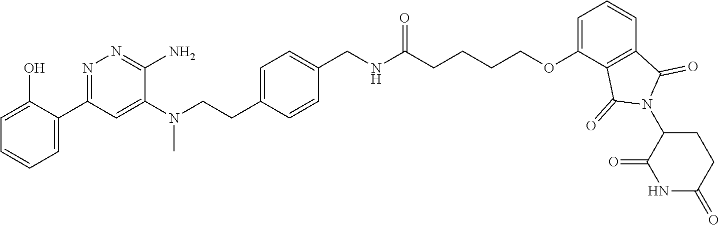 [SMILES: CN(CCc1ccc(CNC(=O)CCCCOc2cccc3c2C(=O)N(C2CCC(=O)NC2=O)C3=O)cc1)c1cc(-c2ccccc2O)nnc1N] |
| 147 | 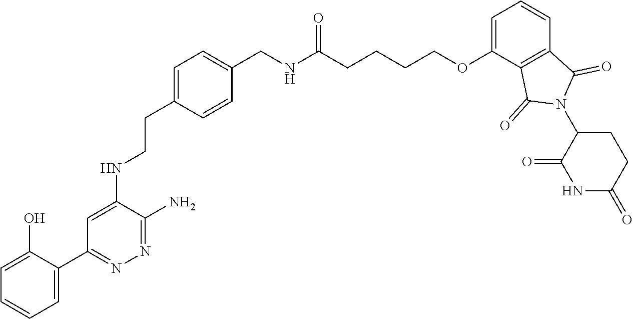 [SMILES: Nc1nnc(-c2ccccc2O)cc1NCCc1ccc(CNC(=O)CCCCOc2cccc3c2C(=O)N(C2CCC(=O)NC2=O)C3=O)cc1] |
| 148 | 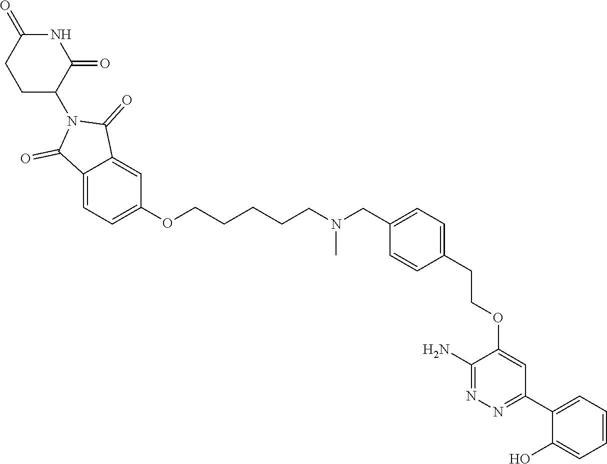 [SMILES: CN(CCCCCOc1ccc2c(c1)C(=O)N(C1CCC(=O)NC1=O)C2=O)Cc1ccc(CCOc2cc(-c3ccccc3O)nnc2N)cc1] |
| 149 | 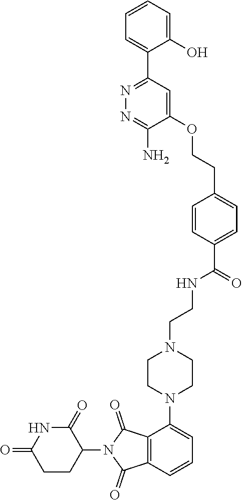 [SMILES: Nc1nnc(-c2ccccc2O)cc1OCCc1ccc(C(=O)NCCN2CCN(c3cccc4c3C(=O)N(C3CCC(=O)NC3=O)C4=O)CC2)cc1] |
| 150 | 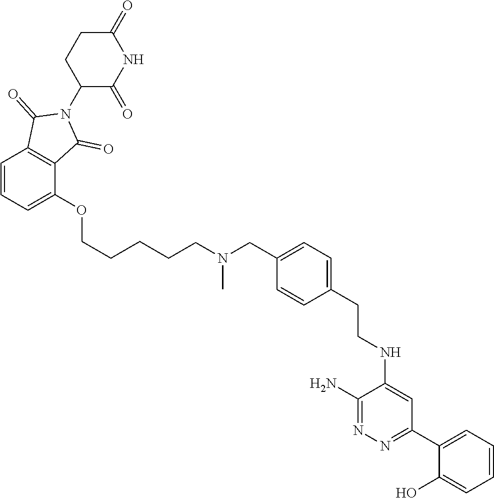 [SMILES: CN(CCCCCOc1cccc2c1C(=O)N(C1CCC(=O)NC1=O)C2=O)Cc1ccc(CCNc2cc(-c3ccccc3O)nnc2N)cc1] |
| 151 | 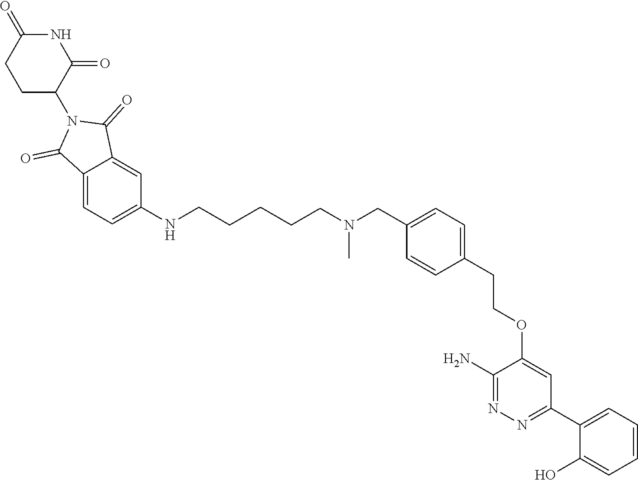 [SMILES: CN(CCCCCNc1ccc2c(c1)C(=O)N(C1CCC(=O)NC1=O)C2=O)Cc1ccc(CCOc2cc(-c3ccccc3O)nnc2N)cc1] |
| 152 | 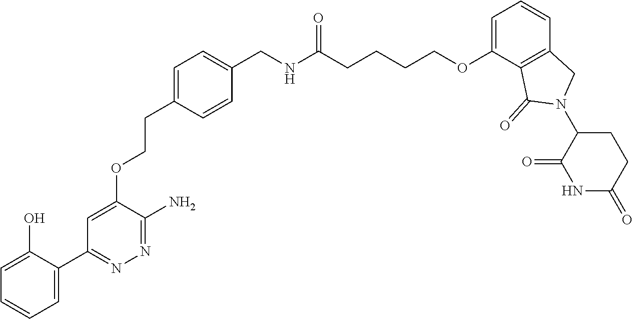 [SMILES: Nc1nnc(-c2ccccc2O)cc1OCCc1ccc(CNC(=O)CCCCOc2cccc3c2C(=O)N(C2CCC(=O)NC2=O)C3)cc1] |
| 153 | 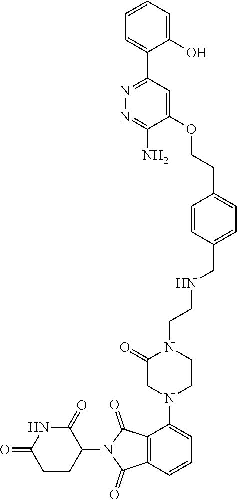 [SMILES: Nc1nnc(-c2ccccc2O)cc1OCCc1ccc(CNCCN2CCN(c3cccc4c3C(=O)N(C3CCC(=O)NC3=O)C4=O)CC2=O)cc1] |
| 154 | 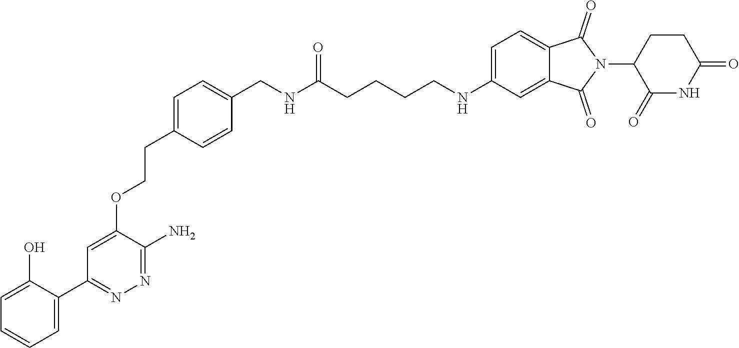 [SMILES: Nc1nnc(-c2ccccc2O)cc1OCCc1ccc(CNC(=O)CCCCNc2ccc3c(c2)C(=O)N(C2CCC(=O)NC2=O)C3=O)cc1] |
| 155 | 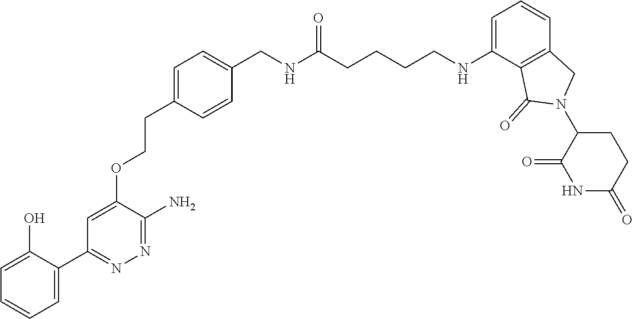 [SMILES: Nc1nnc(-c2ccccc2O)cc1OCCc1ccc(CNC(=O)CCCCNc2cccc3c2C(=O)N(C2CCC(=O)NC2=O)C3)cc1] |
| 156 | 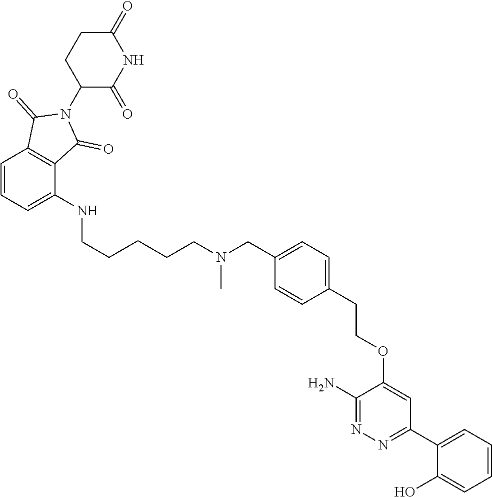 [SMILES: CN(CCCCCNc1cccc2c1C(=O)N(C1CCC(=O)NC1=O)C2=O)Cc1ccc(CCOc2cc(-c3ccccc3O)nnc2N)cc1] |
| 158 | 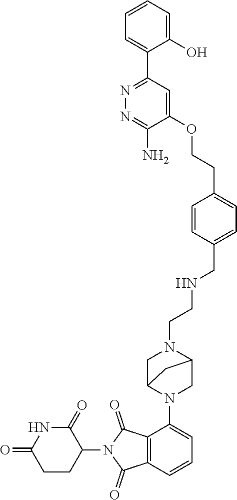 [SMILES: Nc1nnc(-c2ccccc2O)cc1OCCc1ccc(CNCCN2CC3CC2CN3c2cccc3c2C(=O)N(C2CCC(=O)NC2=O)C3=O)cc1] |
| 159 | 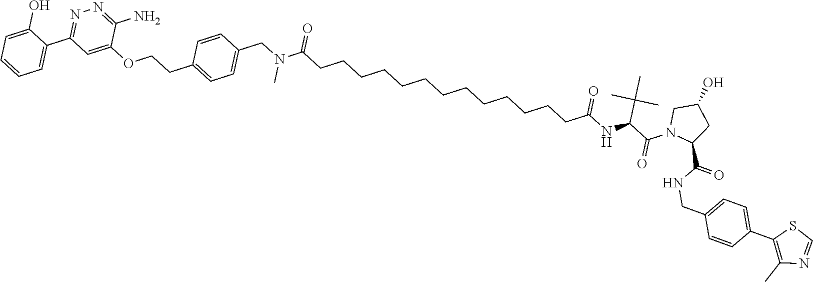 [SMILES: Cc1ncsc1-c1ccc(CNC(=O)[C@@H]2C[C@@H](O)CN2C(=O)[C@@H](NC(=O)CCCCCCCCCCCCCC(=O)N(C)Cc2ccc(CCOc3cc(-c4ccccc4O)nnc3N)cc2)C(C)(C)C)cc1] |
| 160 | 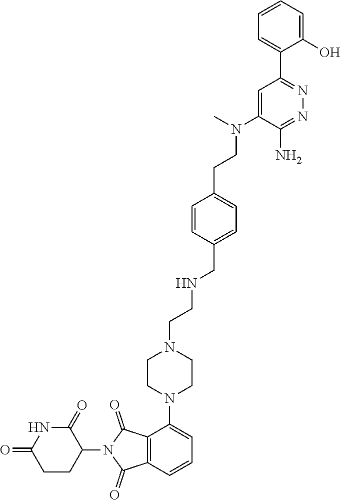 [SMILES: CN(CCc1ccc(CNCCN2CCN(c3cccc4c3C(=O)N(C3CCC(=O)NC3=O)C4=O)CC2)cc1)c1cc(-c2ccccc2O)nnc1N] |
| 161 | 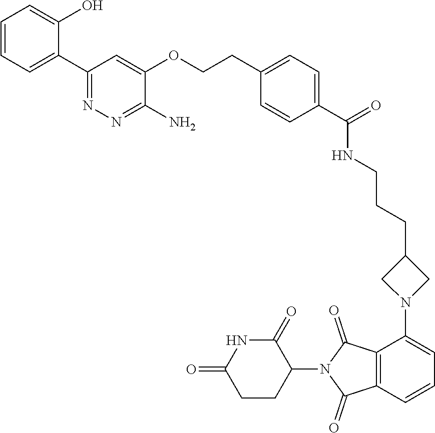 [SMILES: Nc1nnc(-c2ccccc2O)cc1OCCc1ccc(C(=O)NCCCC2CN(c3cccc4c3C(=O)N(C3CCC(=O)NC3=O)C4=O)C2)cc1] |
| 162 | 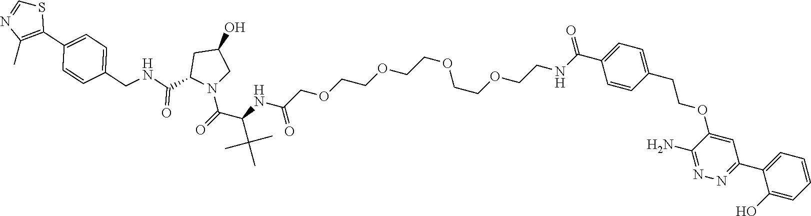 [SMILES: Cc1ncsc1-c1ccc(CNC(=O)[C@@H]2C[C@@H](O)CN2C(=O)[C@@H](NC(=O)COCCOCCOCCOCCNC(=O)c2ccc(CCOc3cc(-c4ccccc4O)nnc3N)cc2)C(C)(C)C)cc1] |
| 163 | 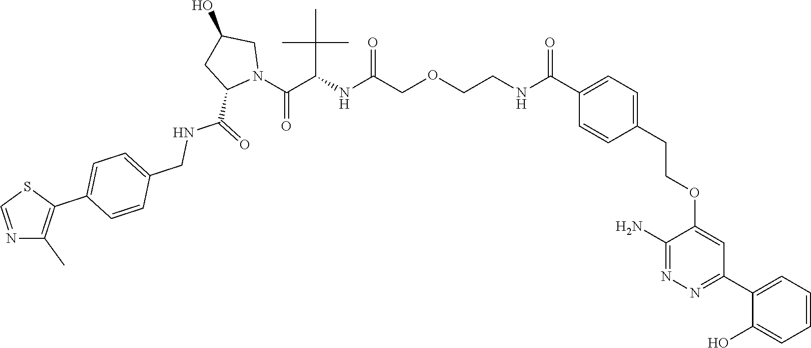 [SMILES: Cc1ncsc1-c1ccc(CNC(=O)[C@@H]2C[C@@H](O)CN2C(=O)[C@@H](NC(=O)COCCNC(=O)c2ccc(CCOc3cc(-c4ccccc4O)nnc3N)cc2)C(C)(C)C)cc1] |
| 164 | 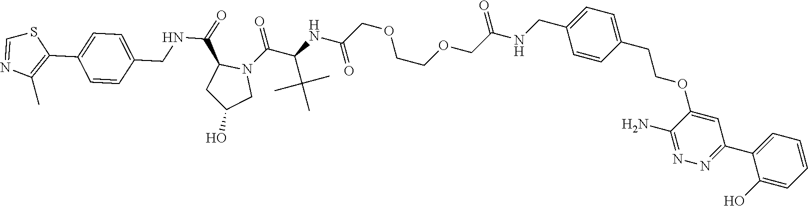 [SMILES: Cc1ncsc1-c1ccc(CNC(=O)[C@@H]2C[C@@H](O)CN2C(=O)[C@@H](NC(=O)COCCOCC(=O)NCc2ccc(CCOc3cc(-c4ccccc4O)nnc3N)cc2)C(C)(C)C)cc1] |
| 165 | 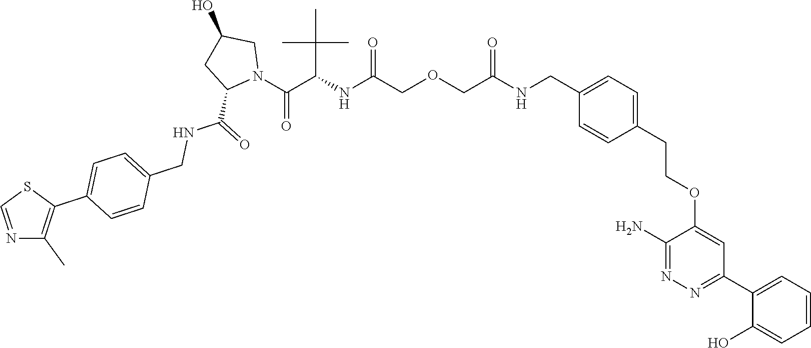 [SMILES: Cc1ncsc1-c1ccc(CNC(=O)[C@@H]2C[C@@H](O)CN2C(=O)[C@@H](NC(=O)COCC(=O)NCc2ccc(CCOc3cc(-c4ccccc4O)nnc3N)cc2)C(C)(C)C)cc1] |
| 166 | 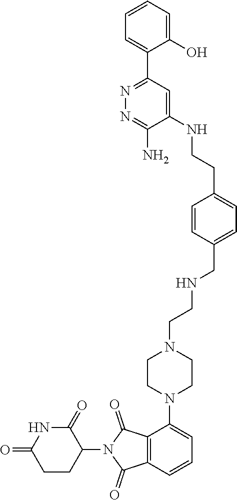 [SMILES: Nc1nnc(-c2ccccc2O)cc1NCCc1ccc(CNCCN2CCN(c3cccc4c3C(=O)N(C3CCC(=O)NC3=O)C4=O)CC2)cc1] |
| 167 | 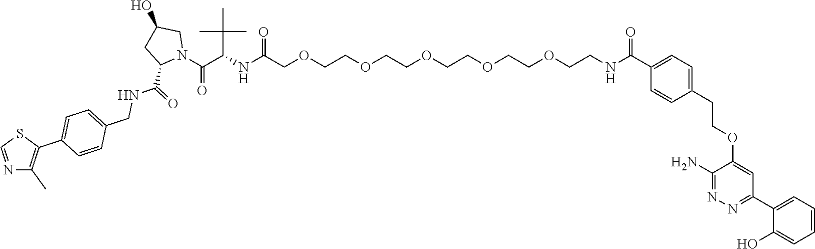 [SMILES: Cc1ncsc1-c1ccc(CNC(=O)[C@@H]2C[C@@H](O)CN2C(=O)[C@@H](NC(=O)COCCOCCOCCOCCOCCNC(=O)c2ccc(CCOc3cc(-c4ccccc4O)nnc3N)cc2)C(C)(C)C)cc1] |
| 168 | 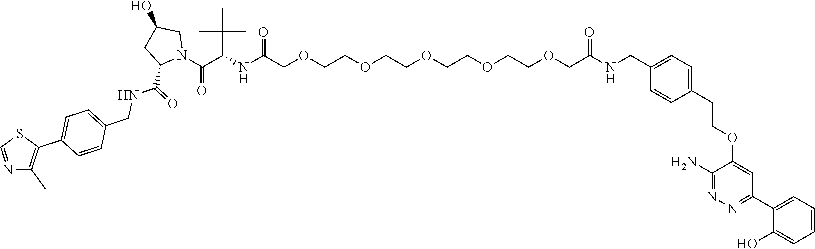 [SMILES: Cc1ncsc1-c1ccc(CNC(=O)[C@@H]2C[C@@H](O)CN2C(=O)[C@@H](NC(=O)COCCOCCOCCOCCOCC(=O)NCc2ccc(CCOc3cc(-c4ccccc4O)nnc3N)cc2)C(C)(C)C)cc1] |
| 169 | 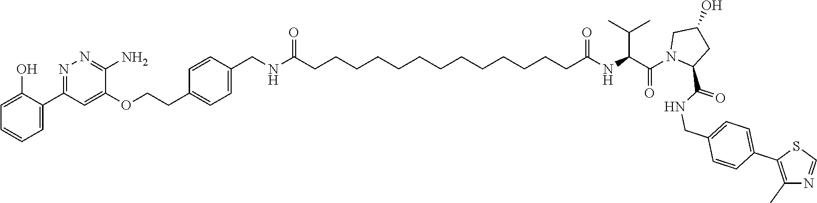 [SMILES: Cc1ncsc1-c1ccc(CNC(=O)[C@@H]2C[C@@H](O)CN2C(=O)[C@@H](NC(=O)CCCCCCCCCCCCCC(=O)NCc2ccc(CCOc3cc(-c4ccccc4O)nnc3N)cc2)C(C)C)cc1] |
| 170 | 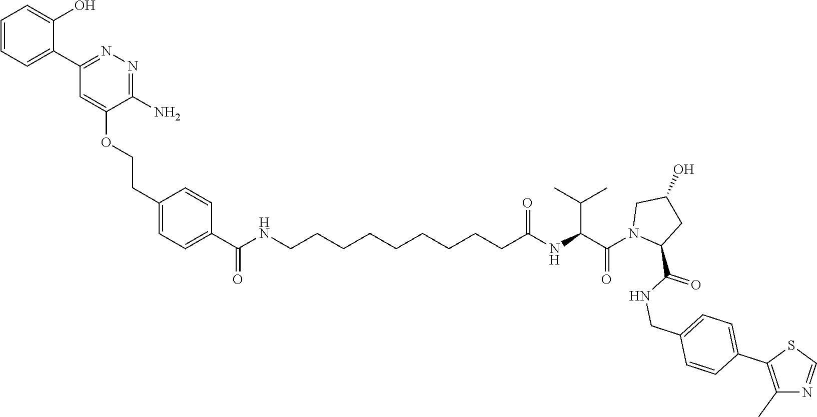 [SMILES: Cc1ncsc1-c1ccc(CNC(=O)[C@@H]2C[C@@H](O)CN2C(=O)[C@@H](NC(=O)CCCCCCCCCNC(=O)c2ccc(CCOc3cc(-c4ccccc4O)nnc3N)cc2)C(C)C)cc1] |
| 171 | 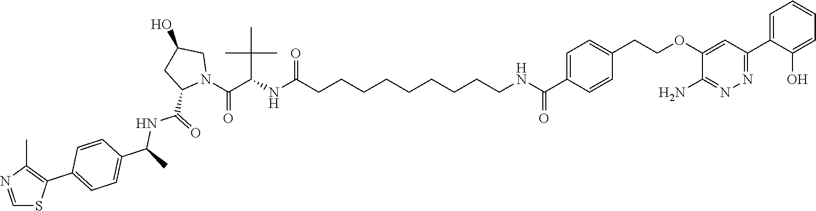 [SMILES: Cc1ncsc1-c1ccc([C@H](C)NC(=O)[C@@H]2C[C@@H](O)CN2C(=O)[C@@H](NC(=O)CCCCCCCCCNC(=O)c2ccc(CCOc3cc(-c4ccccc4O)nnc3N)cc2)C(C)(C)C)cc1] |
| 172 | 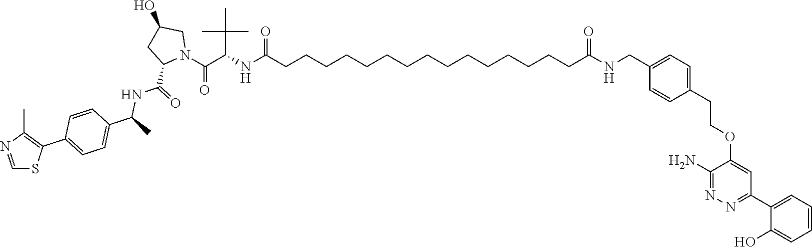 [SMILES: Cc1ncsc1-c1ccc([C@H](C)NC(=O)[C@@H]2C[C@@H](O)CN2C(=O)[C@@H](NC(=O)CCCCCCCCCCCCCCCC(=O)NCc2ccc(CCOc3cc(-c4ccccc4O)nnc3N)cc2)C(C)(C)C)cc1] |
| 173 | 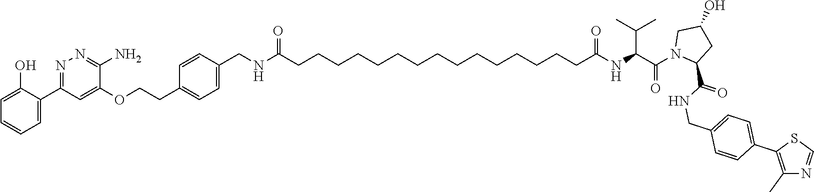 [SMILES: Cc1ncsc1-c1ccc(CNC(=O)[C@@H]2C[C@@H](O)CN2C(=O)[C@@H](NC(=O)CCCCCCCCCCCCCCCC(=O)NCc2ccc(CCOc3cc(-c4ccccc4O)nnc3N)cc2)C(C)C)cc1] |
| 174 | 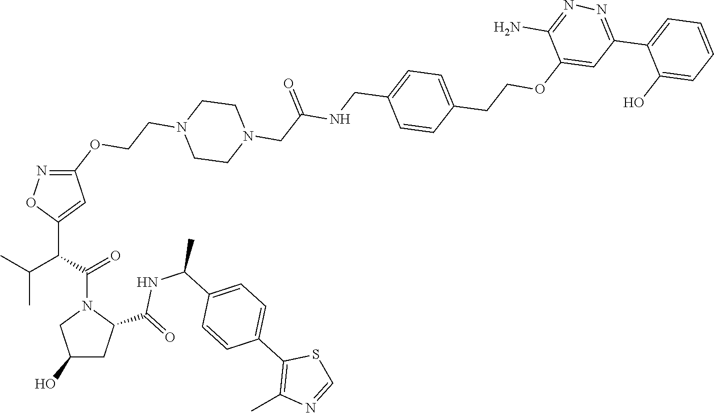 [SMILES: Cc1ncsc1-c1ccc([C@H](C)NC(=O)[C@@H]2C[C@@H](O)CN2C(=O)[C@@H](c2cc(OCCN3CCN(CC(=O)NCc4ccc(CCOc5cc(-c6ccccc6O)nnc5N)cc4)CC3)no2)C(C)C)cc1] |
| 175 | 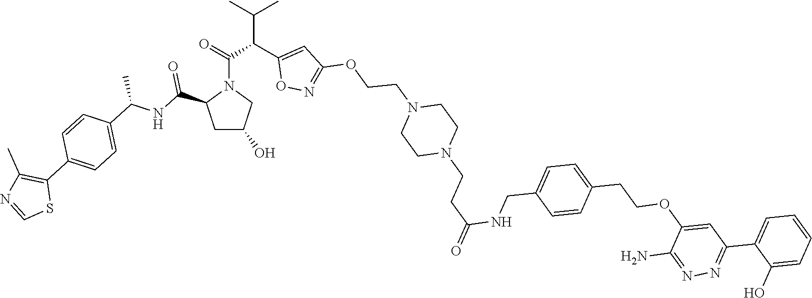 [SMILES: Cc1ncsc1-c1ccc([C@H](C)NC(=O)[C@@H]2C[C@@H](O)CN2C(=O)[C@@H](c2cc(OCCN3CCN(CCC(=O)NCc4ccc(CCOc5cc(-c6ccccc6O)nnc5N)cc4)CC3)no2)C(C)C)cc1] |
| 176 | 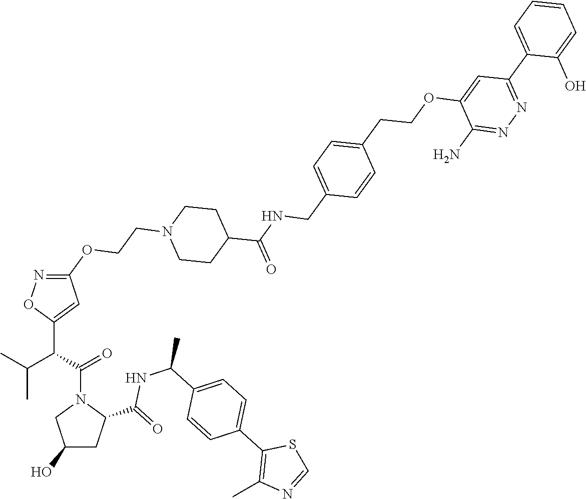 [SMILES: Cc1ncsc1-c1ccc([C@H](C)NC(=O)[C@@H]2C[C@@H](O)CN2C(=O)[C@@H](c2cc(OCCN3CCC(C(=O)NCc4ccc(CCOc5cc(-c6ccccc6O)nnc5N)cc4)CC3)no2)C(C)C)cc1] |
| 177 | 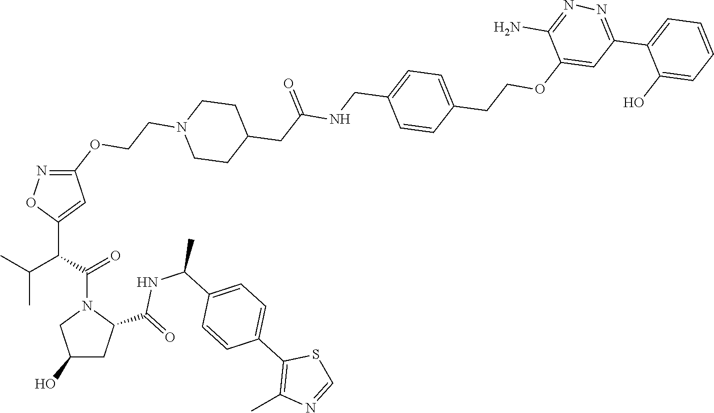 [SMILES: Cc1ncsc1-c1ccc([C@H](C)NC(=O)[C@@H]2C[C@@H](O)CN2C(=O)[C@@H](c2cc(OCCN3CCC(CC(=O)NCc4ccc(CCOc5cc(-c6ccccc6O)nnc5N)cc4)CC3)no2)C(C)C)cc1] |
| 178 | 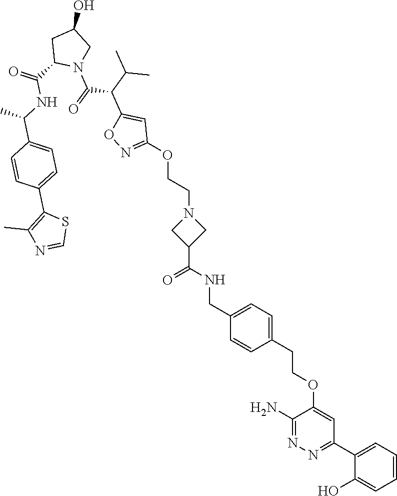 [SMILES: Cc1ncsc1-c1ccc([C@H](C)NC(=O)[C@@H]2C[C@@H](O)CN2C(=O)[C@@H](c2cc(OCCN3CC(C(=O)NCc4ccc(CCOc5cc(-c6ccccc6O)nnc5N)cc4)C3)no2)C(C)C)cc1] |
| 179 | 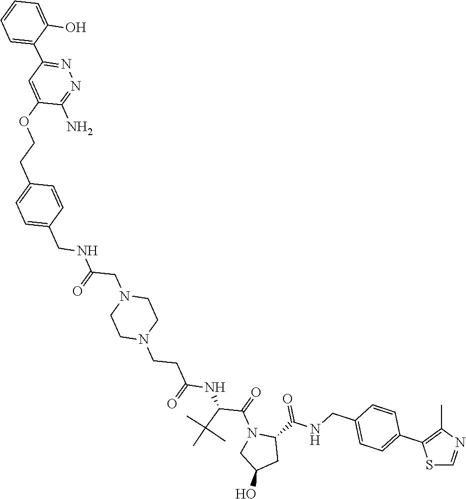 [SMILES: Cc1ncsc1-c1ccc(CNC(=O)[C@@H]2C[C@@H](O)CN2C(=O)[C@@H](NC(=O)CCN2CCN(CC(=O)NCc3ccc(CCOc4cc(-c5ccccc5O)nnc4N)cc3)CC2)C(C)(C)C)cc1] |
| 180 | 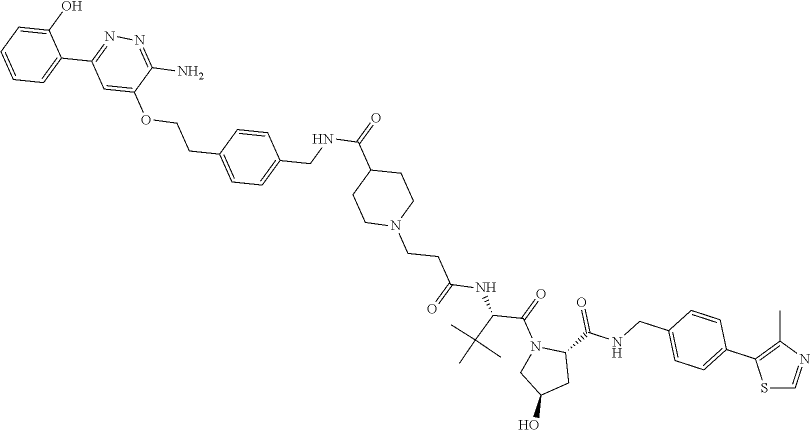 [SMILES: Cc1ncsc1-c1ccc(CNC(=O)[C@@H]2C[C@@H](O)CN2C(=O)[C@@H](NC(=O)CCN2CCC(C(=O)NCc3ccc(CCOc4cc(-c5ccccc5O)nnc4N)cc3)CC2)C(C)(C)C)cc1] |
| 181 | 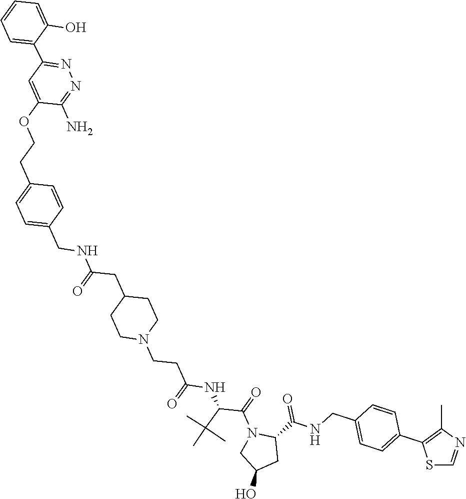 [SMILES: Cc1ncsc1-c1ccc(CNC(=O)[C@@H]2C[C@@H](O)CN2C(=O)[C@@H](NC(=O)CCN2CCC(CC(=O)NCc3ccc(CCOc4cc(-c5ccccc5O)nnc4N)cc3)CC2)C(C)(C)C)cc1] |
| 182 | 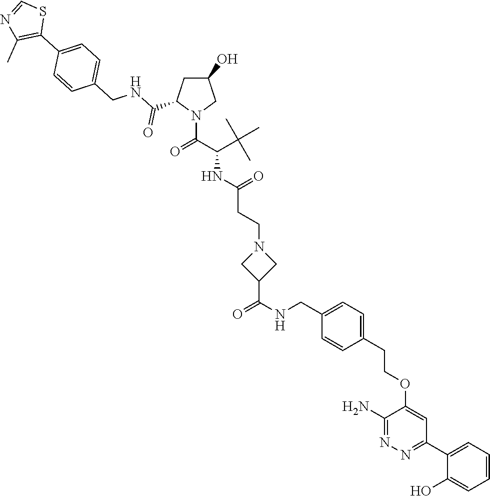 [SMILES: Cc1ncsc1-c1ccc(CNC(=O)[C@@H]2C[C@@H](O)CN2C(=O)[C@@H](NC(=O)CCN2CC(C(=O)NCc3ccc(CCOc4cc(-c5ccccc5O)nnc4N)cc3)C2)C(C)(C)C)cc1] |
| 183 | 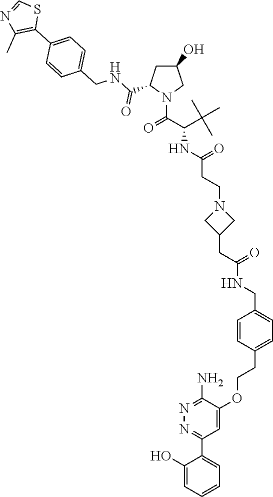 [SMILES: Cc1ncsc1-c1ccc(CNC(=O)[C@@H]2C[C@@H](O)CN2C(=O)[C@@H](NC(=O)CCN2CC(CC(=O)NCc3ccc(CCOc4cc(-c5ccccc5O)nnc4N)cc3)C2)C(C)(C)C)cc1] |
| 184 | 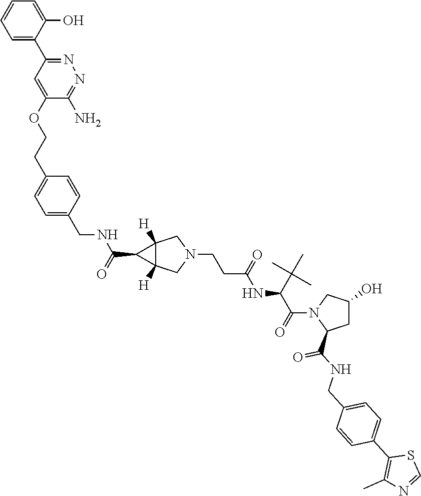 [SMILES: Cc1ncsc1-c1ccc(CNC(=O)[C@@H]2C[C@@H](O)CN2C(=O)[C@@H](NC(=O)CCN2C[C@@H]3[C@H](C2)[C@H]3C(=O)NCc2ccc(CCOc3cc(-c4ccccc4O)nnc3N)cc2)C(C)(C)C)cc1] |
| 185 | 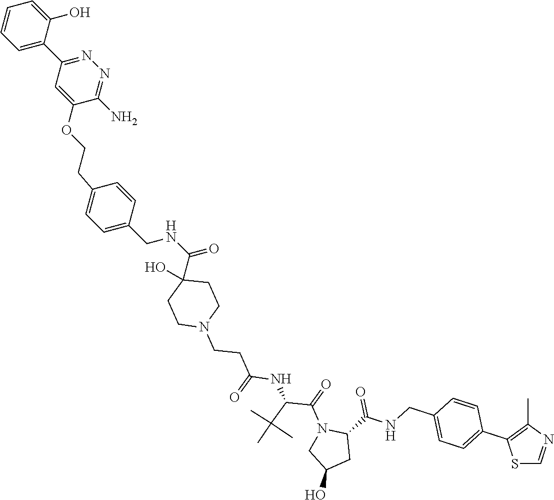 [SMILES: Cc1ncsc1-c1ccc(CNC(=O)[C@@H]2C[C@@H](O)CN2C(=O)[C@@H](NC(=O)CCN2CCC(O)(C(=O)NCc3ccc(CCOc4cc(-c5ccccc5O)nnc4N)cc3)CC2)C(C)(C)C)cc1] |
| 186 | 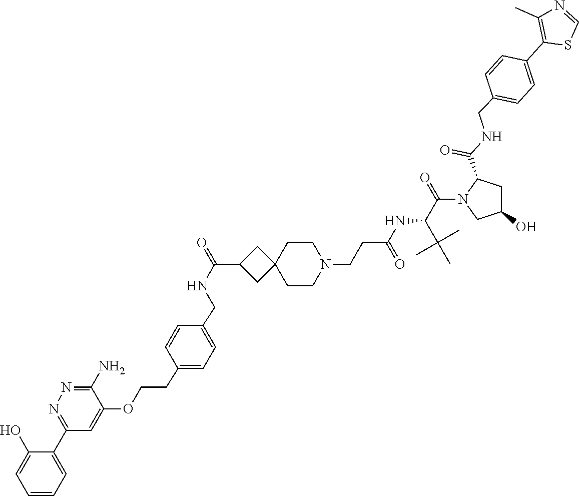 [SMILES: Cc1ncsc1-c1ccc(CNC(=O)[C@@H]2C[C@@H](O)CN2C(=O)[C@@H](NC(=O)CCN2CCC3(CC2)CC(C(=O)NCc2ccc(CCOc4cc(-c5ccccc5O)nnc4N)cc2)C3)C(C)(C)C)cc1] |
| 187 | 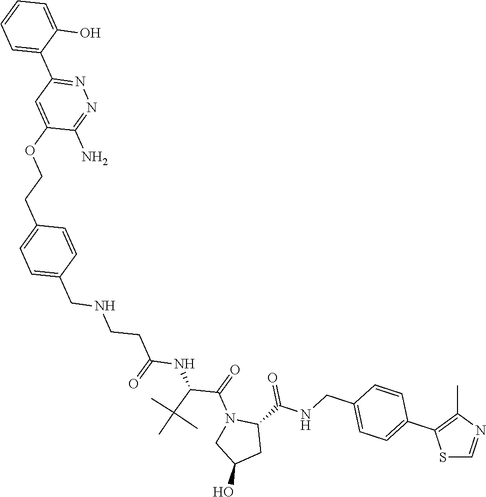 [SMILES: Cc1ncsc1-c1ccc(CNC(=O)[C@@H]2C[C@@H](O)CN2C(=O)[C@@H](NC(=O)CCNCc2ccc(CCOc3cc(-c4ccccc4O)nnc3N)cc2)C(C)(C)C)cc1] |
| 188 | 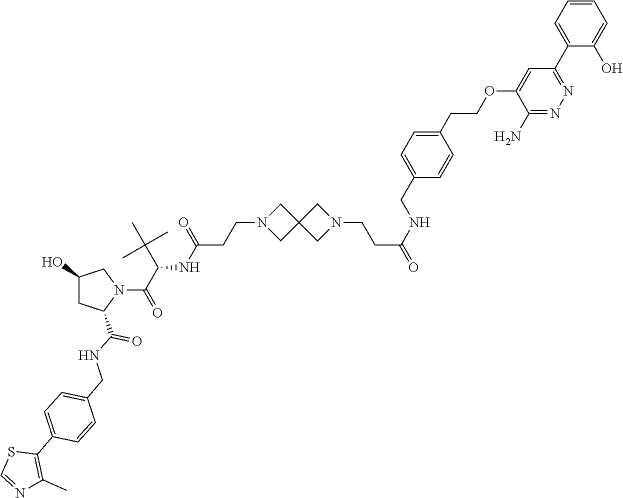 [SMILES: Cc1ncsc1-c1ccc(CNC(=O)[C@@H]2C[C@@H](O)CN2C(=O)[C@@H](NC(=O)CCN2CC3(CN(CCC(=O)NCc4ccc(CCOc5cc(-c6ccccc6O)nnc5N)cc4)C3)C2)C(C)(C)C)cc1] |
| 189 | 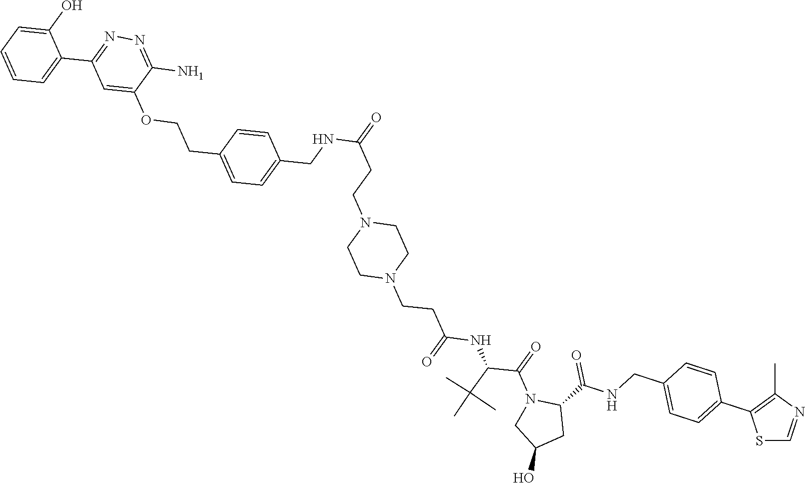 [SMILES: Cc1ncsc1-c1ccc(CNC(=O)[C@@H]2C[C@@H](O)CN2C(=O)[C@@H](NC(=O)CCN2CCN(CCC(=O)NCc3ccc(CCOc4cc(-c5ccccc5O)nnc4N)cc3)CC2)C(C)(C)C)cc1] |
| 190 | 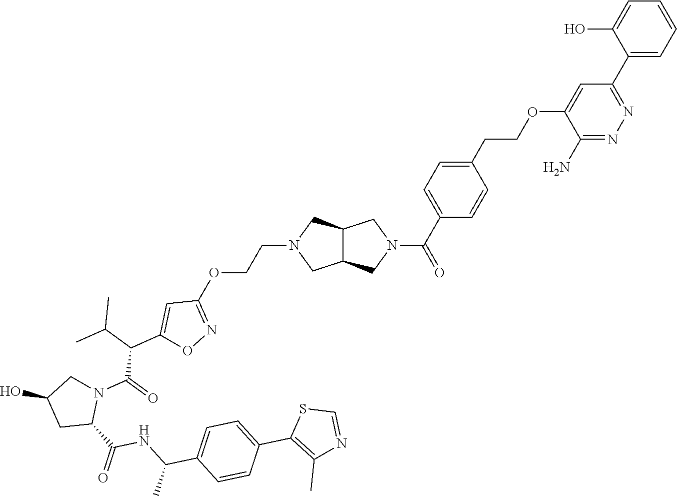 [SMILES: Cc1ncsc1-c1ccc([C@H](C)NC(=O)[C@@H]2C[C@@H](O)CN2C(=O)[C@@H](c2cc(OCCN3C[C@@H]4CN(C(=O)c5ccc(CCOc6cc(-c7ccccc7O)nnc6N)cc5)C[C@@H]4C3)no2)C(C)C)cc1] |
| 191 | 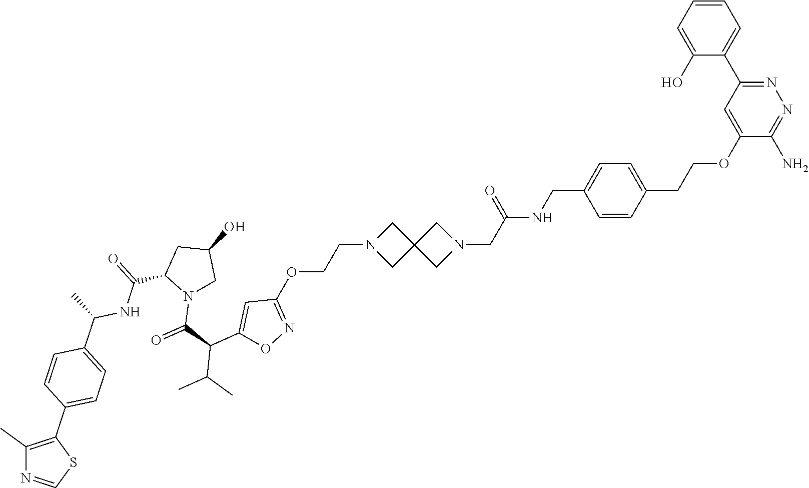 [SMILES: Cc1ncsc1-c1ccc([C@H](C)NC(=O)[C@@H]2C[C@@H](O)CN2C(=O)[C@@H](c2cc(OCCN3CC4(C3)CN(CC(=O)NCc3ccc(CCOc5cc(-c6ccccc6O)nnc5N)cc3)C4)no2)C(C)C)cc1] |
| 192 | 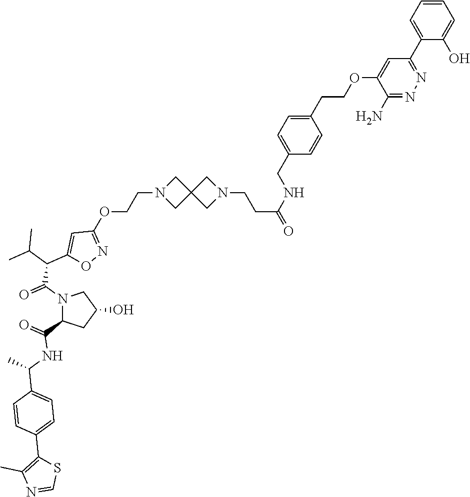 [SMILES: Cc1ncsc1-c1ccc([C@H](C)NC(=O)[C@@H]2C[C@@H](O)CN2C(=O)[C@@H](c2cc(OCCN3CC4(C3)CN(CCC(=O)NCc3ccc(CCOc5cc(-c6ccccc6O)nnc5N)cc3)C4)no2)C(C)C)cc1] |
| 193 | 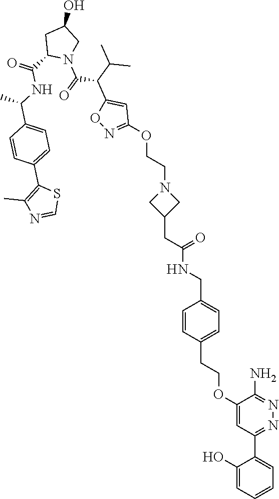 [SMILES: Cc1ncsc1-c1ccc([C@H](C)NC(=O)[C@@H]2C[C@@H](O)CN2C(=O)[C@@H](c2cc(OCCN3CC(CC(=O)NCc4ccc(CCOc5cc(-c6ccccc6O)nnc5N)cc4)C3)no2)C(C)C)cc1] |
| 194 | 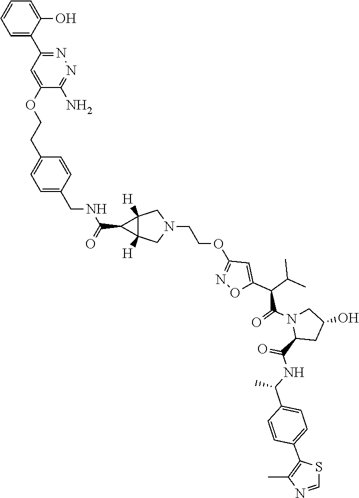 [SMILES: Cc1ncsc1-c1ccc([C@H](C)NC(=O)[C@@H]2C[C@@H](O)CN2C(=O)[C@@H](c2cc(OCCN3C[C@@H]4[C@H](C3)[C@H]4C(=O)NCc3ccc(CCOc4cc(-c5ccccc5O)nnc4N)cc3)no2)C(C)C)cc1] |
| 195 | 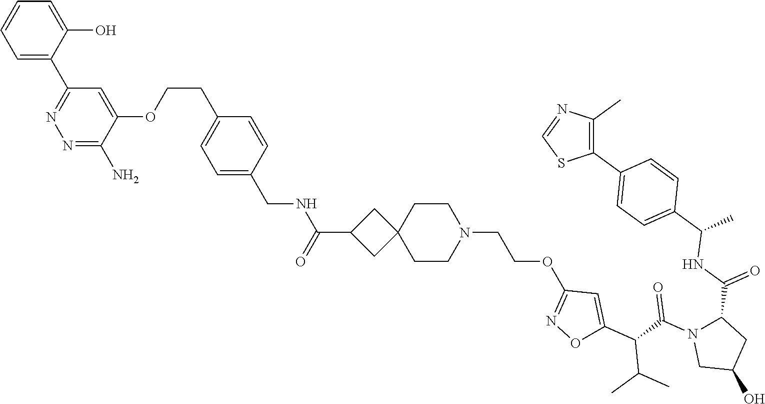 [SMILES: Cc1ncsc1-c1ccc([C@H](C)NC(=O)[C@@H]2C[C@@H](O)CN2C(=O)[C@@H](c2cc(OCCN3CCC4(CC3)CC(C(=O)NCc3ccc(CCOc5cc(-c6ccccc6O)nnc5N)cc3)C4)no2)C(C)C)cc1] |
| 196 | 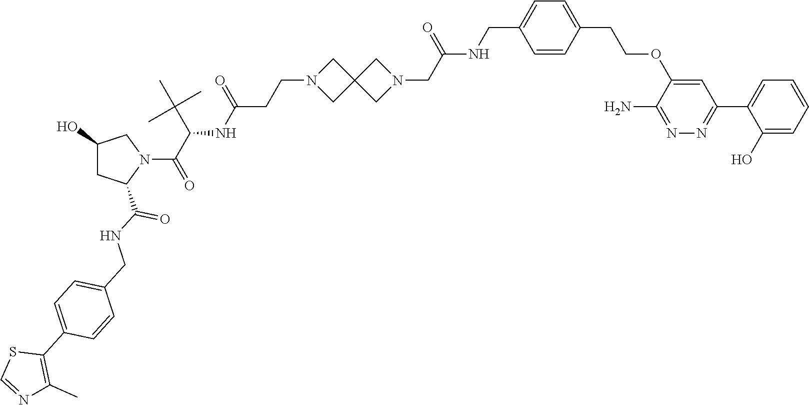 [SMILES: Cc1ncsc1-c1ccc(CNC(=O)[C@@H]2C[C@@H](O)CN2C(=O)[C@@H](NC(=O)CCN2CC3(C2)CN(CC(=O)NCc2ccc(CCOc4cc(-c5ccccc5O)nnc4N)cc2)C3)C(C)(C)C)cc1] |
| 197 | 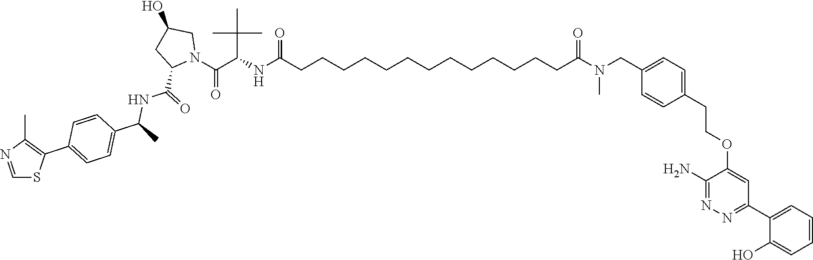 [SMILES: Cc1ncsc1-c1ccc([C@H](C)NC(=O)[C@@H]2C[C@@H](O)CN2C(=O)[C@@H](NC(=O)CCCCCCCCCCCCCC(=O)N(C)Cc2ccc(CCOc3cc(-c4ccccc4O)nnc3N)cc2)C(C)(C)C)cc1] |
| 198 | 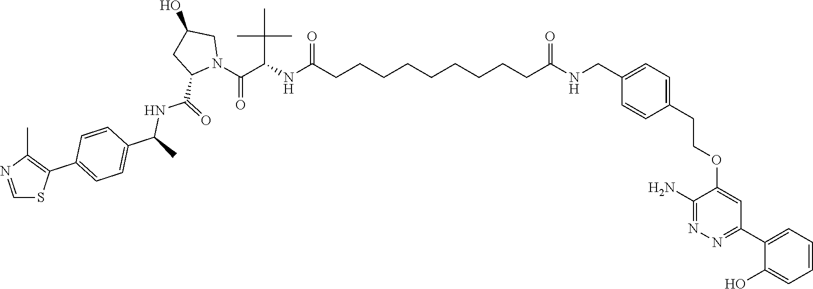 [SMILES: Cc1ncsc1-c1ccc([C@H](C)NC(=O)[C@@H]2C[C@@H](O)CN2C(=O)[C@@H](NC(=O)CCCCCCCCCC(=O)NCc2ccc(CCOc3cc(-c4ccccc4O)nnc3N)cc2)C(C)(C)C)cc1] |
| 199 | 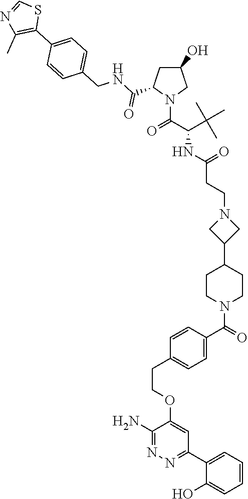 [SMILES: Cc1ncsc1-c1ccc(CNC(=O)[C@@H]2C[C@@H](O)CN2C(=O)[C@@H](NC(=O)CCN2CC(C3CCN(C(=O)c4ccc(CCOc5cc(-c6ccccc6O)nnc5N)cc4)CC3)C2)C(C)(C)C)cc1] |
| 200 | 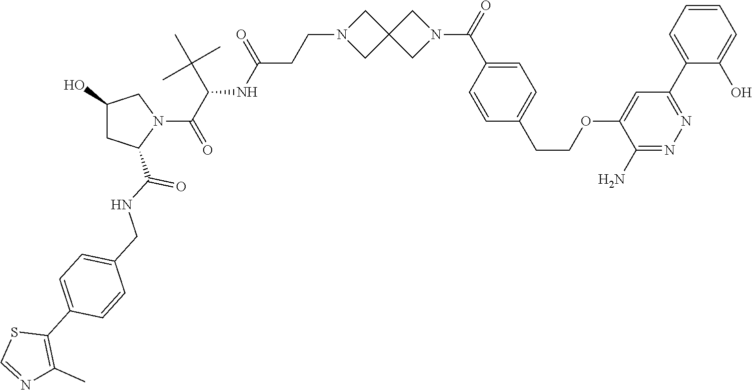 [SMILES: Cc1ncsc1-c1ccc(CNC(=O)[C@@H]2C[C@@H](O)CN2C(=O)[C@@H](NC(=O)CCN2CC3(C2)CN(C(=O)c2ccc(CCOc4cc(-c5ccccc5O)nnc4N)cc2)C3)C(C)(C)C)cc1] |
| 201 | 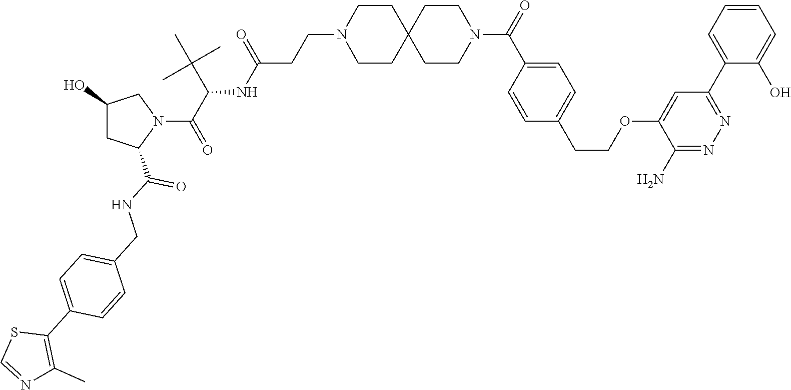 [SMILES: Cc1ncsc1-c1ccc(CNC(=O)[C@@H]2C[C@@H](O)CN2C(=O)[C@@H](NC(=O)CCN2CCC3(CC2)CCN(C(=O)c2ccc(CCOc4cc(-c5ccccc5O)nnc4N)cc2)CC3)C(C)(C)C)cc1] |
| 202 | 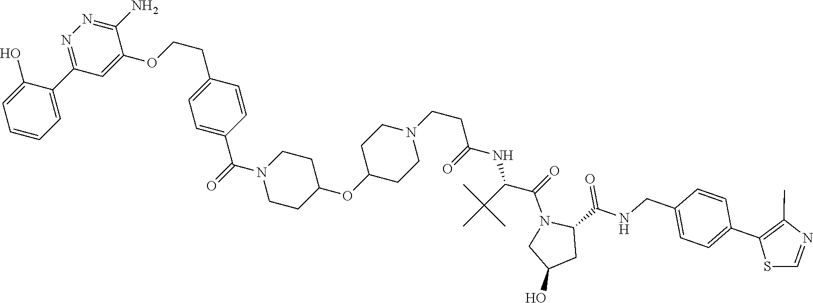 [SMILES: Cc1ncsc1-c1ccc(CNC(=O)[C@@H]2C[C@@H](O)CN2C(=O)[C@@H](NC(=O)CCN2CCC(OC3CCN(C(=O)c4ccc(CCOc5cc(-c6ccccc6O)nnc5N)cc4)CC3)CC2)C(C)(C)C)cc1] |
| 203 | 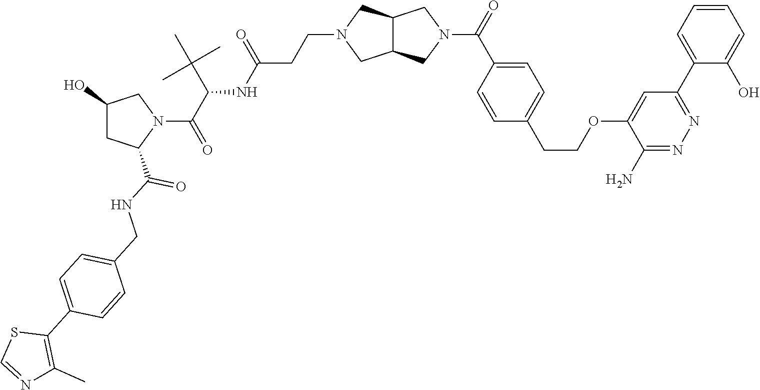 [SMILES: Cc1ncsc1-c1ccc(CNC(=O)[C@@H]2C[C@@H](O)CN2C(=O)[C@@H](NC(=O)CCN2C[C@@H]3CN(C(=O)c4ccc(CCOc5cc(-c6ccccc6O)nnc5N)cc4)C[C@@H]3C2)C(C)(C)C)cc1] |
| 204 | 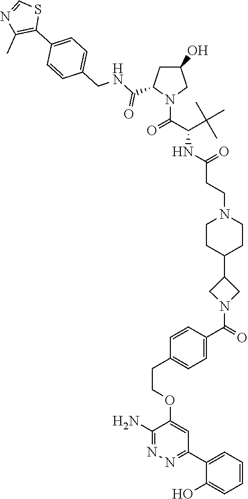 [SMILES: Cc1ncsc1-c1ccc(CNC(=O)[C@@H]2C[C@@H](O)CN2C(=O)[C@@H](NC(=O)CCN2CCC(C3CN(C(=O)c4ccc(CCOc5cc(-c6ccccc6O)nnc5N)cc4)C3)CC2)C(C)(C)C)cc1] |
| 205 | 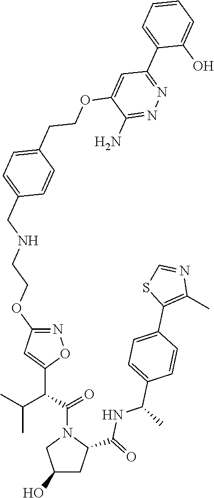 [SMILES: Cc1ncsc1-c1ccc([C@H](C)NC(=O)[C@@H]2C[C@@H](O)CN2C(=O)[C@@H](c2cc(OCCNCc3ccc(CCOc4cc(-c5ccccc5O)nnc4N)cc3)no2)C(C)C)cc1] |
| 206 | 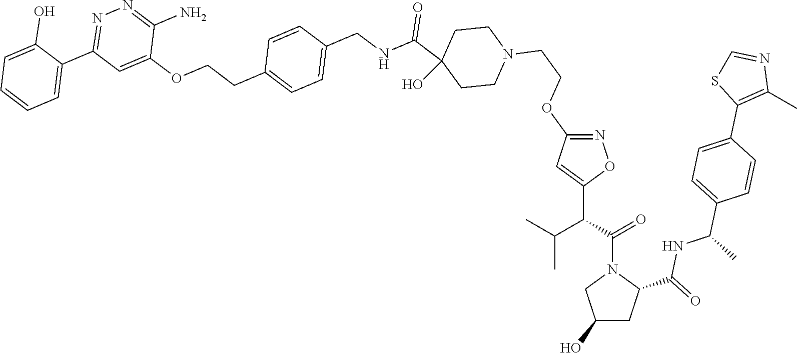 [SMILES: Cc1ncsc1-c1ccc([C@H](C)NC(=O)[C@@H]2C[C@@H](O)CN2C(=O)[C@@H](c2cc(OCCN3CCC(O)(C(=O)NCc4ccc(CCOc5cc(-c6ccccc6O)nnc5N)cc4)CC3)no2)C(C)C)cc1] |
| 207 | 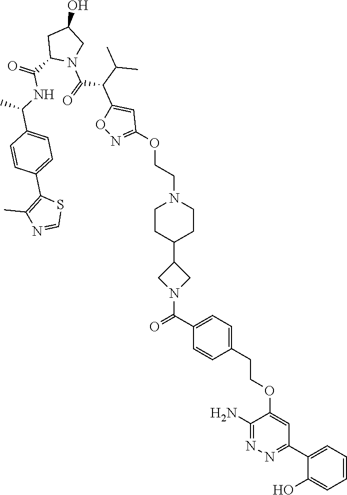 [SMILES: Cc1ncsc1-c1ccc([C@H](C)NC(=O)[C@@H]2C[C@@H](O)CN2C(=O)[C@@H](c2cc(OCCN3CCC(C4CN(C(=O)c5ccc(CCOc6cc(-c7ccccc7O)nnc6N)cc5)C4)CC3)no2)C(C)C)cc1] |
| 208 | 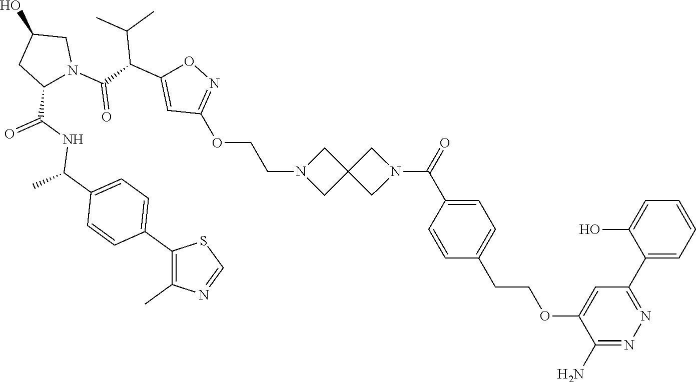 [SMILES: Cc1ncsc1-c1ccc([C@H](C)NC(=O)[C@@H]2C[C@@H](O)CN2C(=O)[C@@H](c2cc(OCCN3CC4(C3)CN(C(=O)c3ccc(CCOc5cc(-c6ccccc6O)nnc5N)cc3)C4)no2)C(C)C)cc1] |
| 209 | 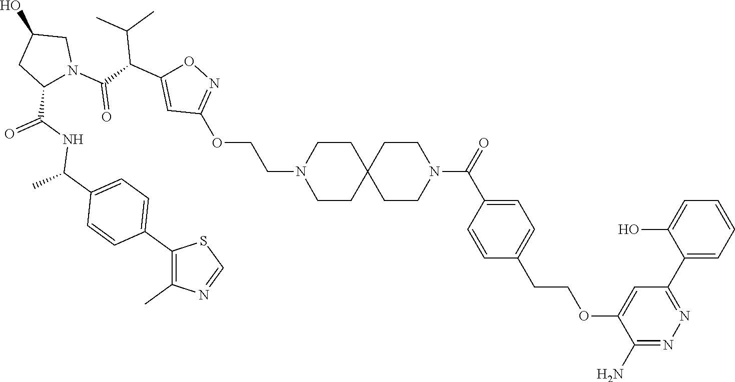 [SMILES: Cc1ncsc1-c1ccc([C@H](C)NC(=O)[C@@H]2C[C@@H](O)CN2C(=O)[C@@H](c2cc(OCCN3CCC4(CC3)CCN(C(=O)c3ccc(CCOc5cc(-c6ccccc6O)nnc5N)cc3)CC4)no2)C(C)C)cc1] |
| 210 | 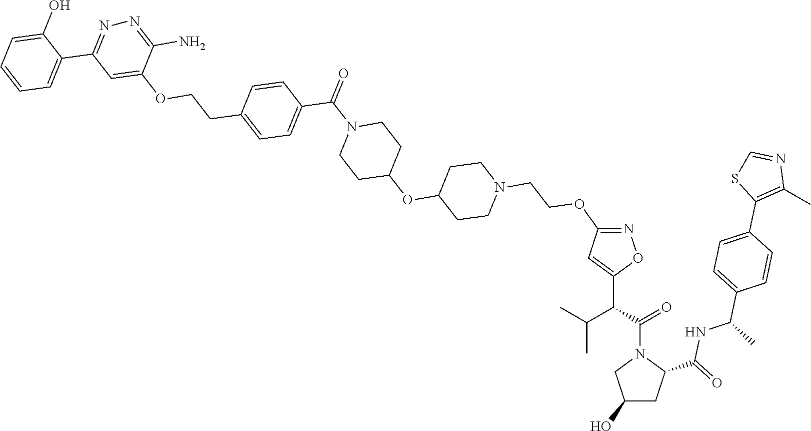 [SMILES: Cc1ncsc1-c1ccc([C@H](C)NC(=O)[C@@H]2C[C@@H](O)CN2C(=O)[C@@H](c2cc(OCCN3CCC(OC4CCN(C(=O)c5ccc(CCOc6cc(-c7ccccc7O)nnc6N)cc5)CC4)CC3)no2)C(C)C)cc1] |
| 211 | 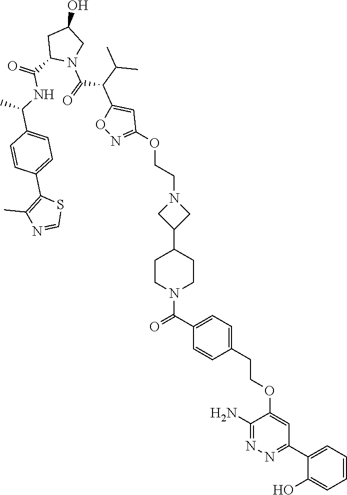 [SMILES: Cc1ncsc1-c1ccc([C@H](C)NC(=O)[C@@H]2C[C@@H](O)CN2C(=O)[C@@H](c2cc(OCCN3CC(C4CCN(C(=O)c5ccc(CCOc6cc(-c7ccccc7O)nnc6N)cc5)CC4)C3)no2)C(C)C)cc1] |
| 212 | 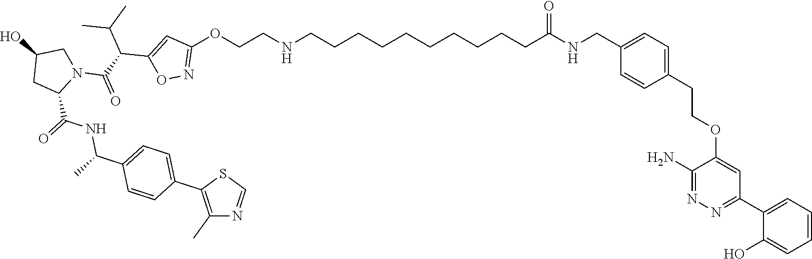 [SMILES: Cc1ncsc1-c1ccc([C@H](C)NC(=O)[C@@H]2C[C@@H](O)CN2C(=O)[C@@H](c2cc(OCCNCCCCCCCCCCC(=O)NCc3ccc(CCOc4cc(-c5ccccc5O)nnc4N)cc3)no2)C(C)C)cc1] |
| 213 | 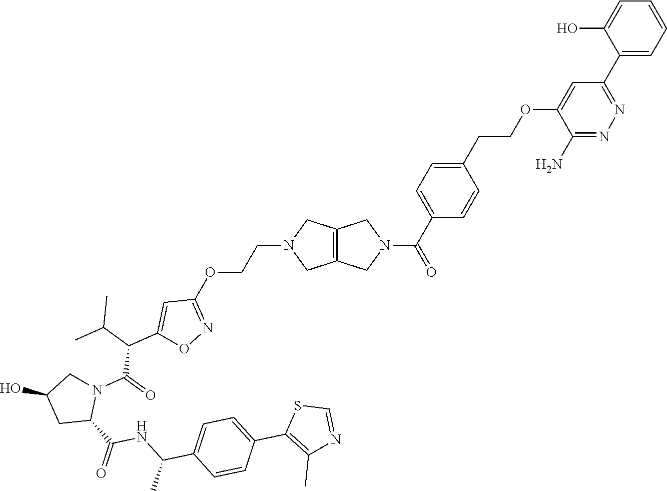 [SMILES: Cc1ncsc1-c1ccc([C@H](C)NC(=O)[C@@H]2C[C@@H](O)CN2C(=O)[C@@H](c2cc(OCCN3CC4=C(C3)CN(C(=O)c3ccc(CCOc5cc(-c6ccccc6O)nnc5N)cc3)C4)no2)C(C)C)cc1] |
| 214 | 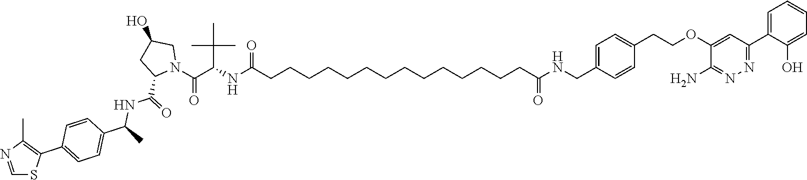 [SMILES: Cc1ncsc1-c1ccc([C@H](C)NC(=O)[C@@H]2C[C@@H](O)CN2C(=O)[C@@H](NC(=O)CCCCCCCCCCCCCCC(=O)NCc2ccc(CCOc3cc(-c4ccccc4O)nnc3N)cc2)C(C)(C)C)cc1] |
| 215 | 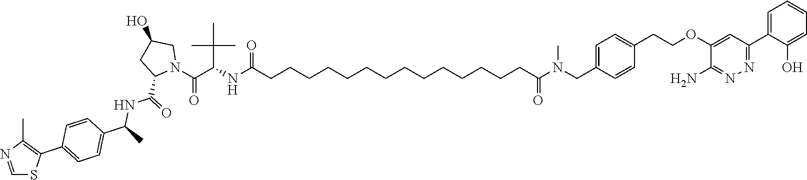 [SMILES: Cc1ncsc1-c1ccc([C@H](C)NC(=O)[C@@H]2C[C@@H](O)CN2C(=O)[C@@H](NC(=O)CCCCCCCCCCCCCCC(=O)N(C)Cc2ccc(CCOc3cc(-c4ccccc4O)nnc3N)cc2)C(C)(C)C)cc1] |
| 216 | 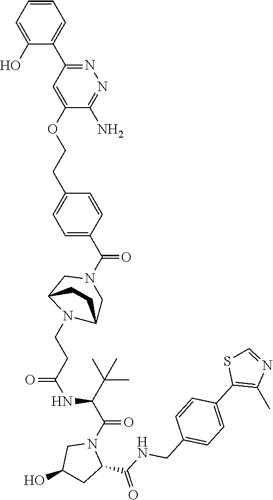 |
| 217 | 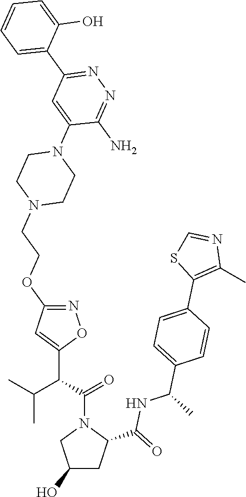 [SMILES: Cc1ncsc1-c1ccc([C@H](C)NC(=O)[C@@H]2C[C@@H](O)CN2C(=O)[C@@H](c2cc(OCCN3CCN(c4cc(-c5ccccc5O)nnc4N)CC3)no2)C(C)C)cc1] |
| 218 | 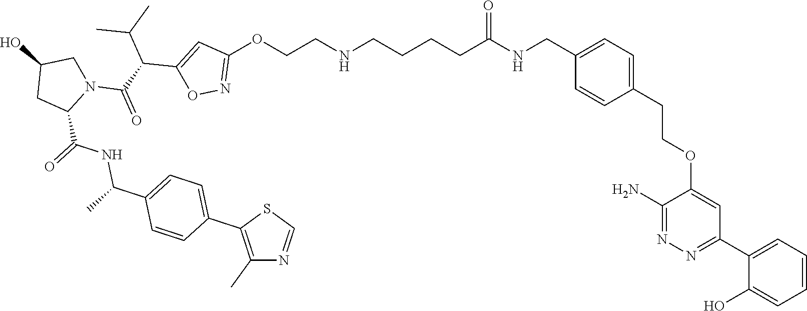 [SMILES: Cc1ncsc1-c1ccc([C@H](C)NC(=O)[C@@H]2C[C@@H](O)CN2C(=O)[C@@H](c2cc(OCCNCCCCC(=O)NCc3ccc(CCOc4cc(-c5ccccc5O)nnc4N)cc3)no2)C(C)C)cc1] |
| 219 | 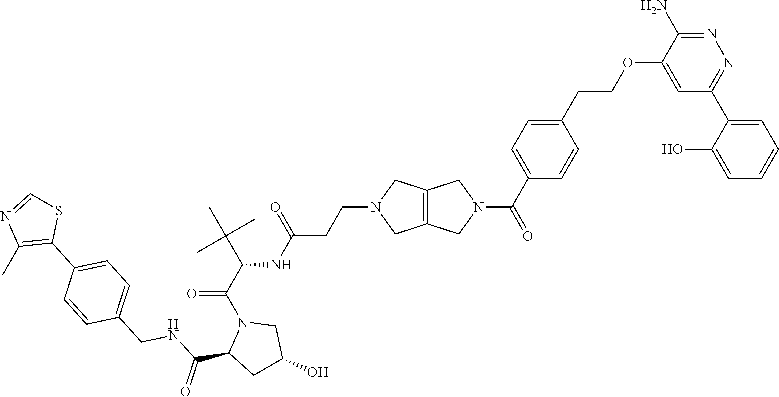 [SMILES: Cc1ncsc1-c1ccc(CNC(=O)[C@@H]2C[C@@H](O)CN2C(=O)[C@@H](NC(=O)CCN2CC3=C(C2)CN(C(=O)c2ccc(CCOc4cc(-c5ccccc5O)nnc4N)cc2)C3)C(C)(C)C)cc1] |
| 220 | 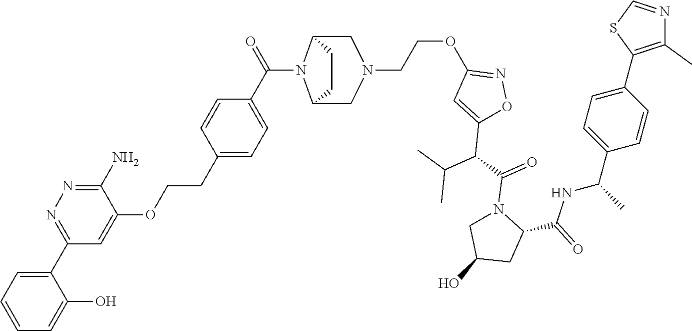 [SMILES: Cc1ncsc1-c1ccc([C@H](C)NC(=O)[C@@H]2C[C@@H](O)CN2C(=O)[C@@H](c2cc(OCCN3C[C@H]4CC[C@@H](C3)N4C(=O)c3ccc(CCOc4cc(-c5ccccc5O)nnc4N)cc3)no2)C(C)C)cc1] |
| 221 | 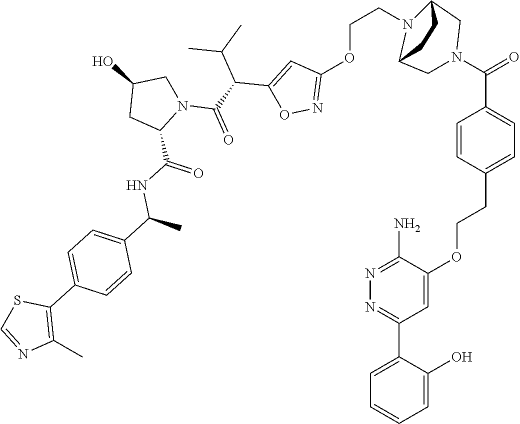 |
| 222 | 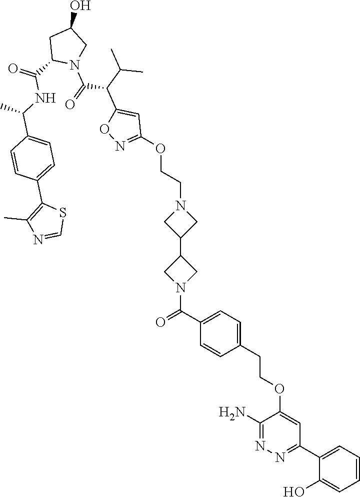 [SMILES: Cc1ncsc1-c1ccc([C@H](C)NC(=O)[C@@H]2C[C@@H](O)CN2C(=O)[C@@H](c2cc(OCCN3CC(C4CN(C(=O)c5ccc(CCOc6cc(-c7ccccc7O)nnc6N)cc5)C4)C3)no2)C(C)C)cc1] |
| 223 | 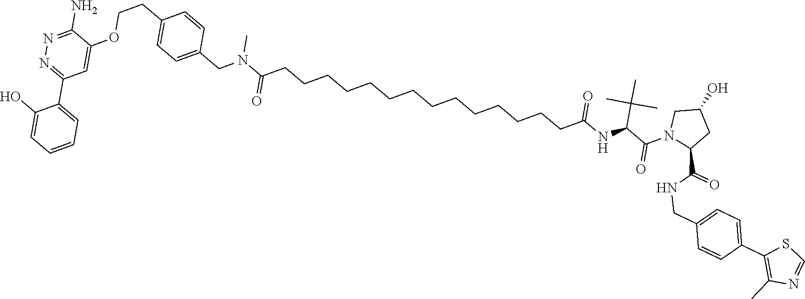 [SMILES: Cc1ncsc1-c1ccc(CNC(=O)[C@@H]2C[C@@H](O)CN2C(=O)[C@@H](NC(=O)CCCCCCCCCCCCCCC(=O)N(C)Cc2ccc(CCOc3cc(-c4ccccc4O)nnc3N)cc2)C(C)(C)C)cc1] |
| 224 | 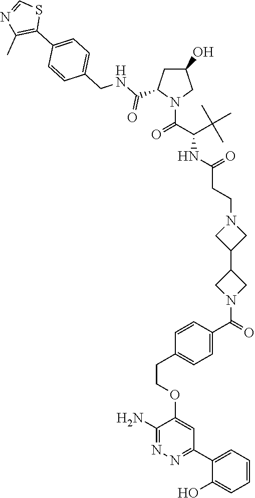 [SMILES: Cc1ncsc1-c1ccc(CNC(=O)[C@@H]2C[C@@H](O)CN2C(=O)[C@@H](NC(=O)CCN2CC(C3CN(C(=O)c4ccc(CCOc5cc(-c6ccccc6O)nnc5N)cc4)C3)C2)C(C)(C)C)cc1] |
| 225 | 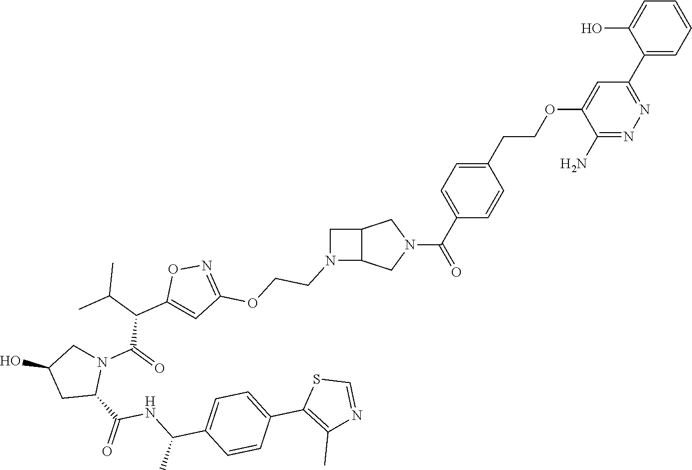 [SMILES: Cc1ncsc1-c1ccc([C@H](C)NC(=O)[C@@H]2C[C@@H](O)CN2C(=O)[C@@H](c2cc(OCCN3CC4CN(C(=O)c5ccc(CCOc6cc(-c7ccccc7O)nnc6N)cc5)CC43)no2)C(C)C)cc1] |
| 226 | 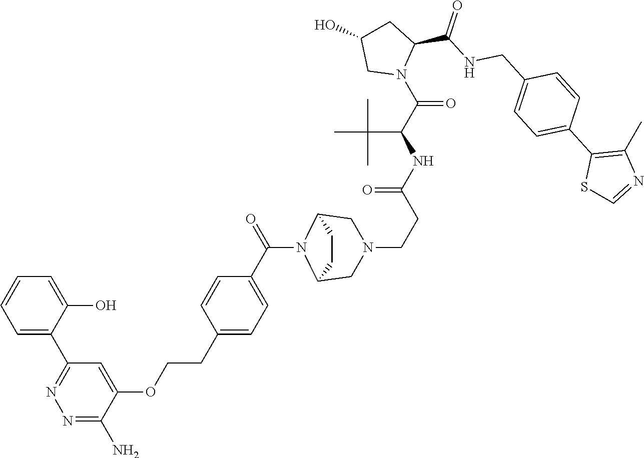 [SMILES: Cc1ncsc1-c1ccc(CNC(=O)[C@@H]2C[C@@H](O)CN2C(=O)[C@@H](NC(=O)CCN2C[C@H]3CC[C@@H](C2)N3C(=O)c2ccc(CCOc3cc(-c4ccccc4O)nnc3N)cc2)C(C)(C)C)cc1] |
| 227 | 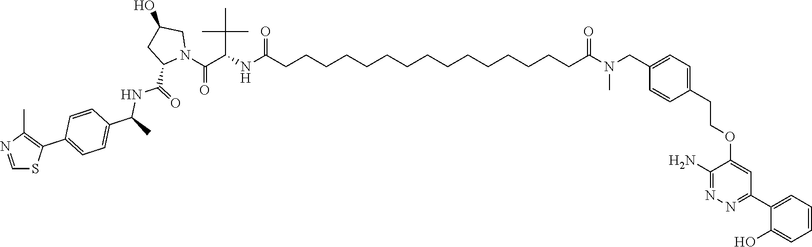 [SMILES: Cc1ncsc1-c1ccc([C@H](C)NC(=O)[C@@H]2C[C@@H](O)CN2C(=O)[C@@H](NC(=O)CCCCCCCCCCCCCCCC(=O)N(C)Cc2ccc(CCOc3cc(-c4ccccc4O)nnc3N)cc2)C(C)(C)C)cc1] |
| 228 | 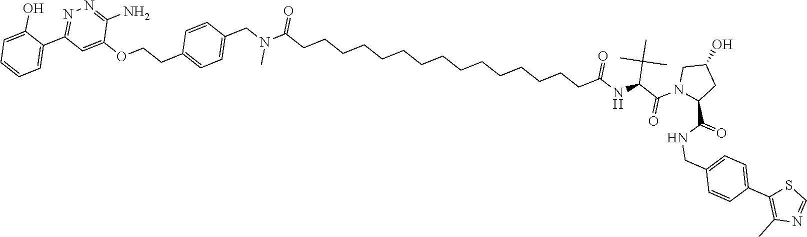 [SMILES: Cc1ncsc1-c1ccc(CNC(=O)[C@@H]2C[C@@H](O)CN2C(=O)[C@@H](NC(=O)CCCCCCCCCCCCCCCC(=O)N(C)Cc2ccc(CCOc3cc(-c4ccccc4O)nnc3N)cc2)C(C)(C)C)cc1] |
| 229 | 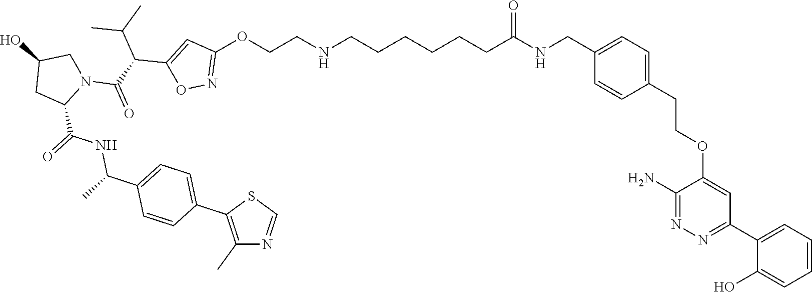 [SMILES: Cc1ncsc1-c1ccc([C@H](C)NC(=O)[C@@H]2C[C@@H](O)CN2C(=O)[C@@H](c2cc(OCCNCCCCCCC(=O)NCc3ccc(CCOc4cc(-c5ccccc5O)nnc4N)cc3)no2)C(C)C)cc1] |
| 230 | 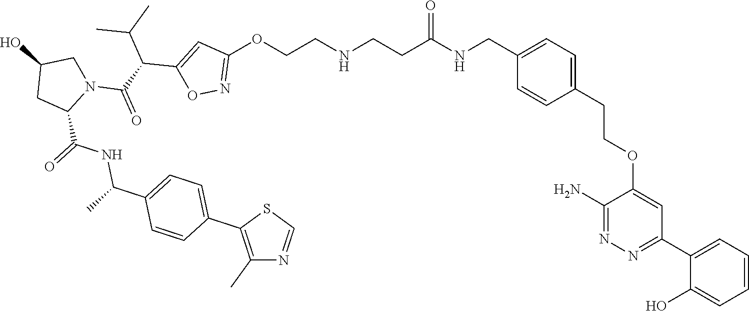 [SMILES: Cc1ncsc1-c1ccc([C@H](C)NC(=O)[C@@H]2C[C@@H](O)CN2C(=O)[C@@H](c2cc(OCCNCCC(=O)NCc3ccc(CCOc4cc(-c5ccccc5O)nnc4N)cc3)no2)C(C)C)cc1] |
| 231 | 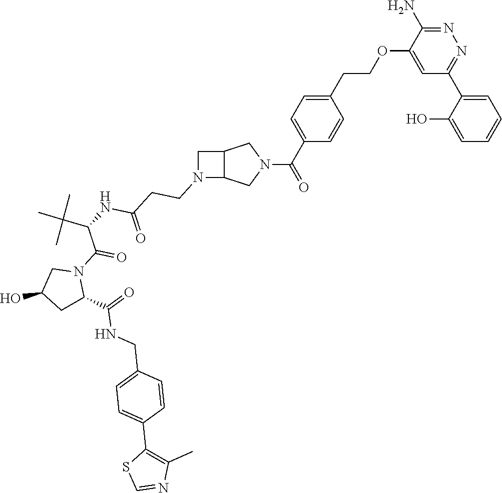 [SMILES: Cc1ncsc1-c1ccc(CNC(=O)[C@@H]2C[C@@H](O)CN2C(=O)[C@@H](NC(=O)CCN2CC3CN(C(=O)c4ccc(CCOc5cc(-c6ccccc6O)nnc5N)cc4)CC32)C(C)(C)C)cc1] |
| 232 | 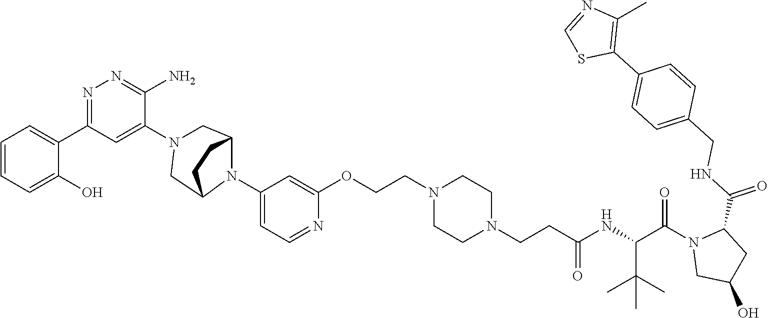 |
| 233 | 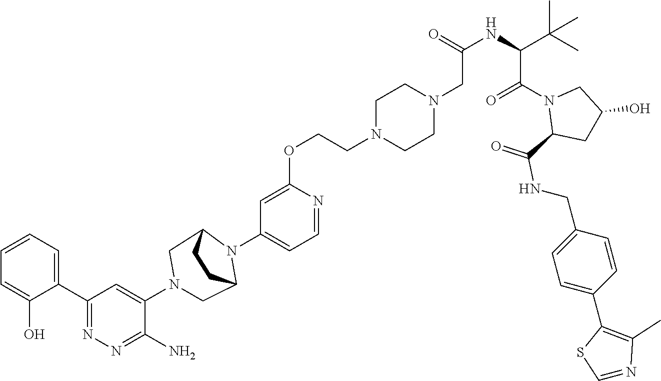 |
| 234 | 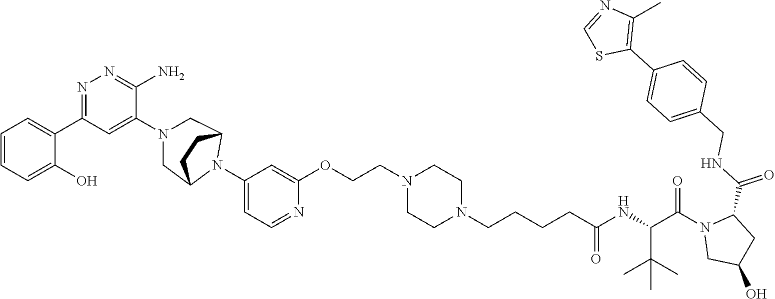 |
| 235 | 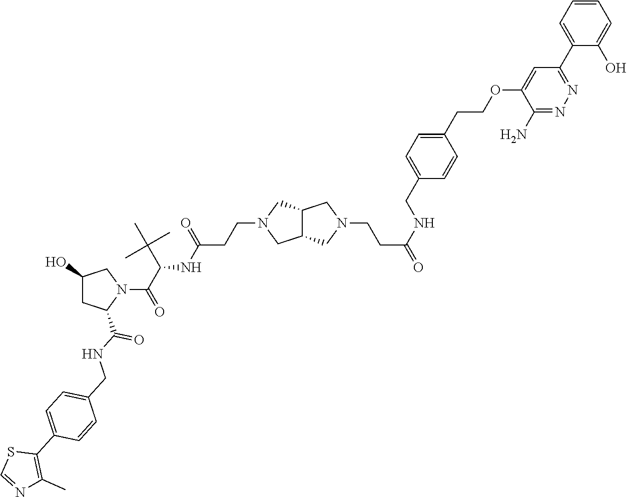 [SMILES: Cc1ncsc1-c1ccc(CNC(=O)[C@@H]2C[C@@H](O)CN2C(=O)[C@@H](NC(=O)CCN2C[C@H]3CN(CCC(=O)NCc4ccc(CCOc5cc(-c6ccccc6O)nnc5N)cc4)C[C@H]3C2)C(C)(C)C)cc1] |
| 236 | 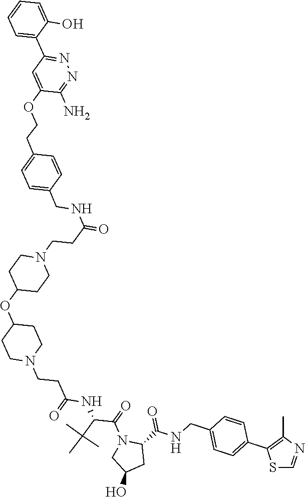 [SMILES: Cc1ncsc1-c1ccc(CNC(=O)[C@@H]2C[C@@H](O)CN2C(=O)[C@@H](NC(=O)CCN2CCC(OC3CCN(CCC(=O)NCc4ccc(CCOc5cc(-c6ccccc6O)nnc5N)cc4)CC3)CC2)C(C)(C)C)cc1] |
| 237 | 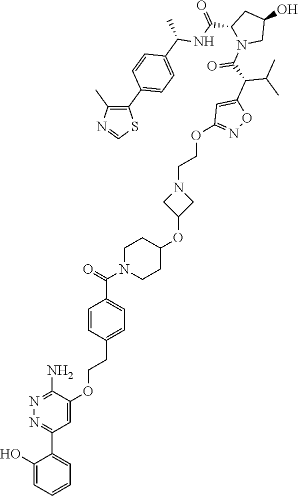 [SMILES: Cc1ncsc1-c1ccc([C@H](C)NC(=O)[C@@H]2C[C@@H](O)CN2C(=O)[C@@H](c2cc(OCCN3CC(OC4CCN(C(=O)c5ccc(CCOc6cc(-c7ccccc7O)nnc6N)cc5)CC4)C3)no2)C(C)C)cc1] |
| 238 | 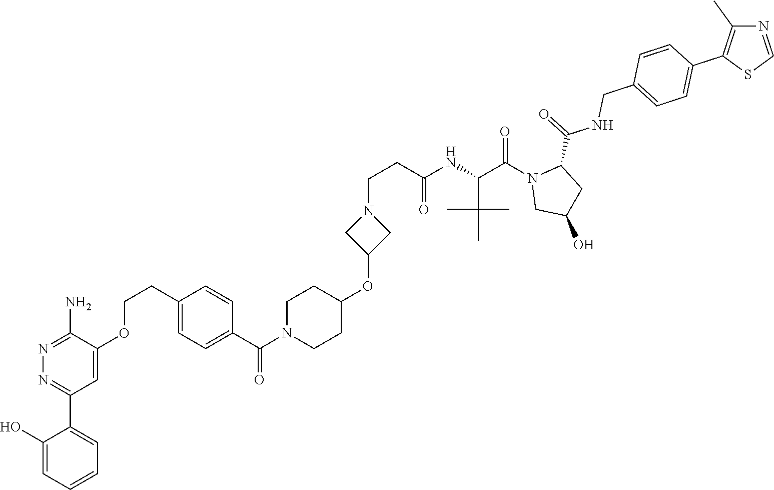 [SMILES: Cc1ncsc1-c1ccc(CNC(=O)[C@@H]2C[C@@H](O)CN2C(=O)[C@@H](NC(=O)CCN2CC(OC3CCN(C(=O)c4ccc(CCOc5cc(-c6ccccc6O)nnc5N)cc4)CC3)C2)C(C)(C)C)cc1] |
| 239 | 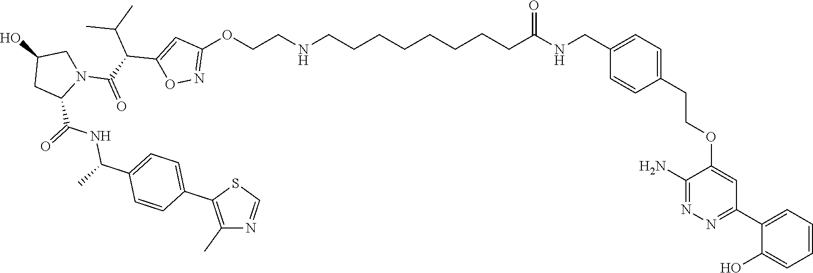 [SMILES: Cc1ncsc1-c1ccc([C@H](C)NC(=O)[C@@H]2C[C@@H](O)CN2C(=O)[C@@H](c2cc(OCCNCCCCCCCCC(=O)NCc3ccc(CCOc4cc(-c5ccccc5O)nnc4N)cc3)no2)C(C)C)cc1] |
| 240 | 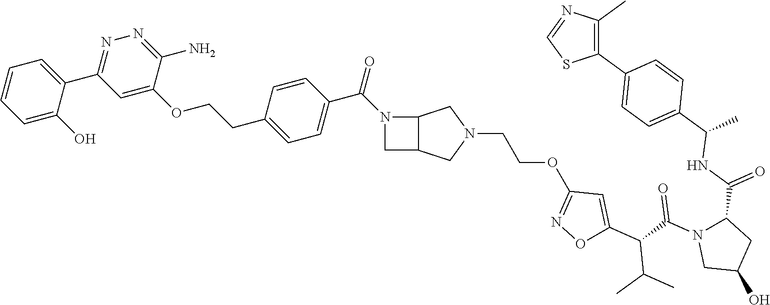 [SMILES: Cc1ncsc1-c1ccc([C@H](C)NC(=O)[C@@H]2C[C@@H](O)CN2C(=O)[C@@H](c2cc(OCCN3CC4CN(C(=O)c5ccc(CCOc6cc(-c7ccccc7O)nnc6N)cc5)C4C3)no2)C(C)C)cc1] |
| 241 | 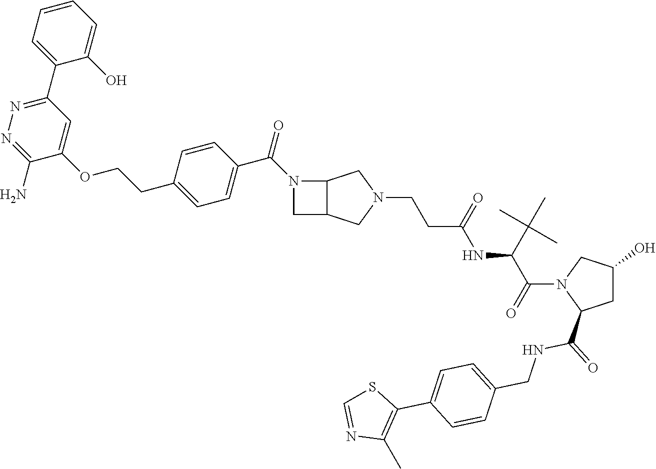 [SMILES: Cc1ncsc1-c1ccc(CNC(=O)[C@@H]2C[C@@H](O)CN2C(=O)[C@@H](NC(=O)CCN2CC3CN(C(=O)c4ccc(CCOc5cc(-c6ccccc6O)nnc5N)cc4)C3C2)C(C)(C)C)cc1] |
| 242 | 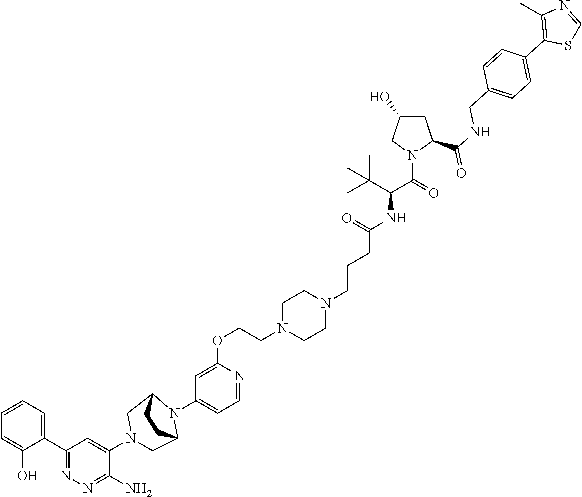 |
| 243 | 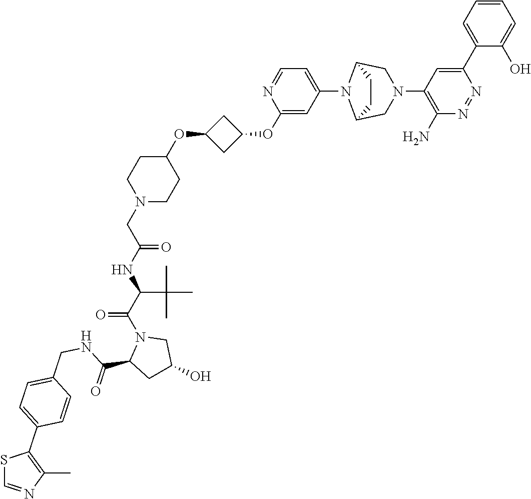 [SMILES: Cc1ncsc1-c1ccc(CNC(=O)[C@@H]2C[C@@H](O)CN2C(=O)[C@@H](NC(=O)CN2CCC(O[C@H]3C[C@H](Oc4cc(N5[C@@H]6CC[C@H]5CN(c5cc(-c7ccccc7O)nnc5N)C6)ccn4)C3)CC2)C(C)(C)C)cc1] |
| 244 | 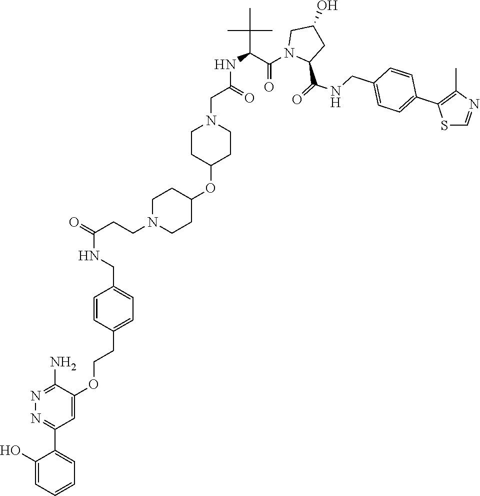 [SMILES: Cc1ncsc1-c1ccc(CNC(=O)[C@@H]2C[C@@H](O)CN2C(=O)[C@@H](NC(=O)CN2CCC(OC3CCN(CCC(=O)NCc4ccc(CCOc5cc(-c6ccccc6O)nnc5N)cc4)CC3)CC2)C(C)(C)C)cc1] |
| 245 | 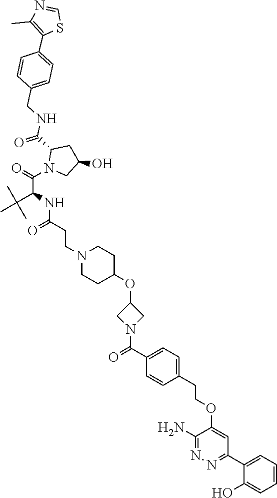 [SMILES: Cc1ncsc1-c1ccc(CNC(=O)[C@@H]2C[C@@H](O)CN2C(=O)[C@@H](NC(=O)CCN2CCC(OC3CN(C(=O)c4ccc(CCOc5cc(-c6ccccc6O)nnc5N)cc4)C3)CC2)C(C)(C)C)cc1] |
| 246 | 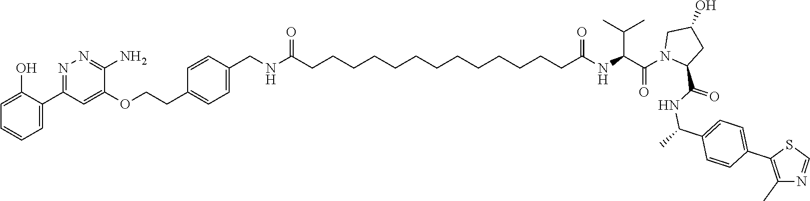 [SMILES: Cc1ncsc1-c1ccc([C@H](C)NC(=O)[C@@H]2C[C@@H](O)CN2C(=O)[C@@H](NC(=O)CCCCCCCCCCCCCC(=O)NCc2ccc(CCOc3cc(-c4ccccc4O)nnc3N)cc2)C(C)C)cc1] |
| 247 | 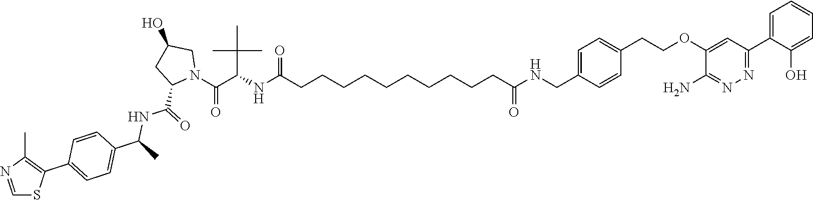 [SMILES: Cc1ncsc1-c1ccc([C@H](C)NC(=O)[C@@H]2C[C@@H](O)CN2C(=O)[C@@H](NC(=O)CCCCCCCCCCC(=O)NCc2ccc(CCOc3cc(-c4ccccc4O)nnc3N)cc2)C(C)(C)C)cc1] |
| 248 | 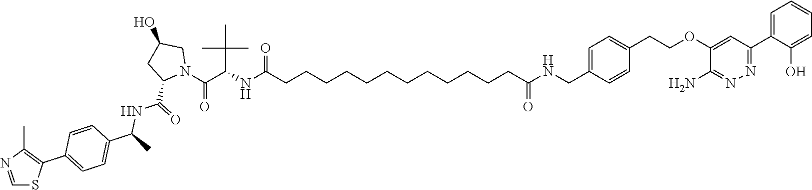 [SMILES: Cc1ncsc1-c1ccc([C@H](C)NC(=O)[C@@H]2C[C@@H](O)CN2C(=O)[C@@H](NC(=O)CCCCCCCCCCCCC(=O)NCc2ccc(CCOc3cc(-c4ccccc4O)nnc3N)cc2)C(C)(C)C)cc1] |
| 249 | 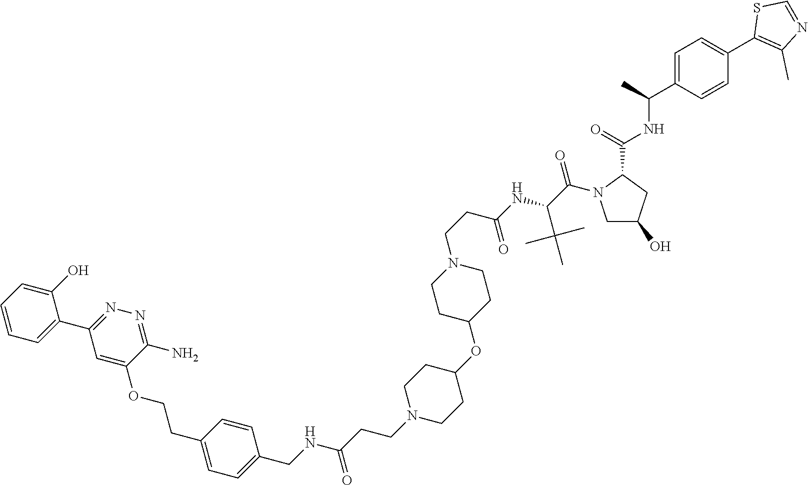 [SMILES: Cc1ncsc1-c1ccc([C@H](C)NC(=O)[C@@H]2C[C@@H](O)CN2C(=O)[C@@H](NC(=O)CCN2CCC(OC3CCN(CCC(=O)NCc4ccc(CCOc5cc(-c6ccccc6O)nnc5N)cc4)CC3)CC2)C(C)(C)C)cc1] |
| 250 | 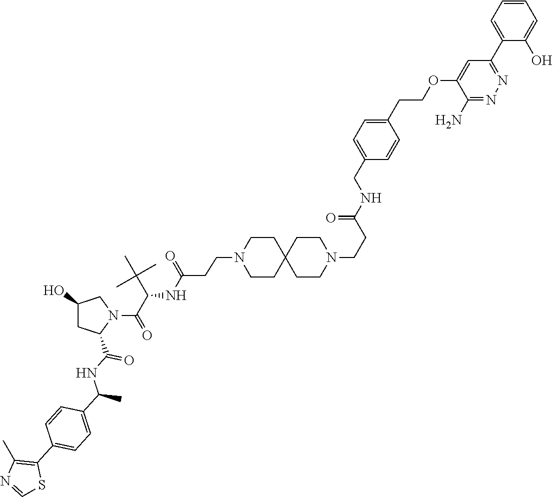 [SMILES: Cc1ncsc1-c1ccc([C@H](C)NC(=O)[C@@H]2C[C@@H](O)CN2C(=O)[C@@H](NC(=O)CCN2CCC3(CCN(CCC(=O)NCc4ccc(CCOc5cc(-c6ccccc6O)nnc5N)cc4)CC3)CC2)C(C)(C)C)cc1] |
| 251 | 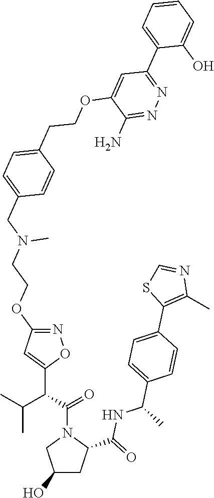 [SMILES: Cc1ncsc1-c1ccc([C@H](C)NC(=O)[C@@H]2C[C@@H](O)CN2C(=O)[C@@H](c2cc(OCCN(C)Cc3ccc(CCOc4cc(-c5ccccc5O)nnc4N)cc3)no2)C(C)C)cc1] |
| 252 | 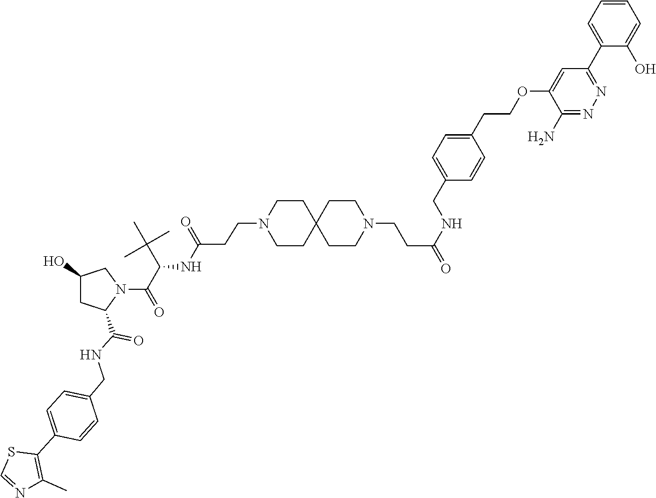 [SMILES: Cc1ncsc1-c1ccc(CNC(=O)[C@@H]2C[C@@H](O)CN2C(=O)[C@@H](NC(=O)CCN2CCC3(CCN(CCC(=O)NCc4ccc(CCOc5cc(-c6ccccc6O)nnc5N)cc4)CC3)CC2)C(C)(C)C)cc1] |
| 253 | 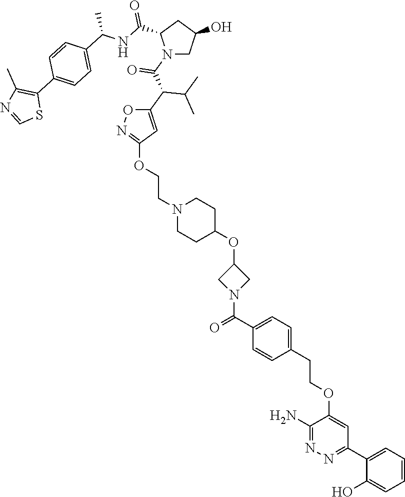 [SMILES: Cc1ncsc1-c1ccc([C@H](C)NC(=O)[C@@H]2C[C@@H](O)CN2C(=O)[C@@H](c2cc(OCCN3CCC(OC4CN(C(=O)c5ccc(CCOc6cc(-c7ccccc7O)nnc6N)cc5)C4)CC3)no2)C(C)C)cc1] |
| 254 | 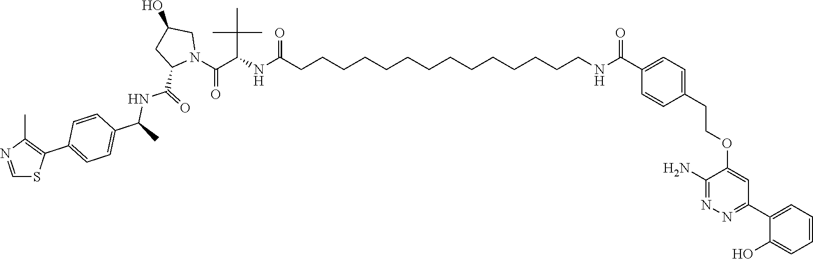 [SMILES: Cc1ncsc1-c1ccc([C@H](C)NC(=O)[C@@H]2C[C@@H](O)CN2C(=O)[C@@H](NC(=O)CCCCCCCCCCCCCCNC(=O)c2ccc(CCOc3cc(-c4ccccc4O)nnc3N)cc2)C(C)(C)C)cc1] |
| 255 | 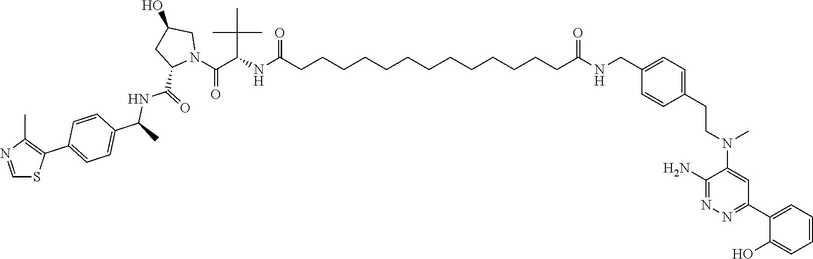 [SMILES: Cc1ncsc1-c1ccc([C@H](C)NC(=O)[C@@H]2C[C@@H](O)CN2C(=O)[C@@H](NC(=O)CCCCCCCCCCCCCC(=O)NCc2ccc(CCN(C)c3cc(-c4ccccc4O)nnc3N)cc2)C(C)(C)C)cc1] |
| 256 | 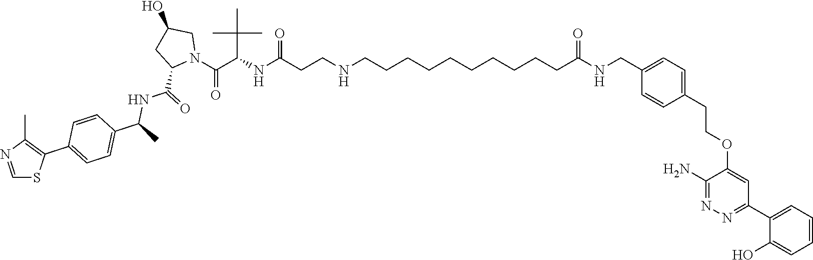 [SMILES: Cc1ncsc1-c1ccc([C@H](C)NC(=O)[C@@H]2C[C@@H](O)CN2C(=O)[C@@H](NC(=O)CCNCCCCCCCCCCC(=O)NCc2ccc(CCOc3cc(-c4ccccc4O)nnc3N)cc2)C(C)(C)C)cc1] |
| 257 | 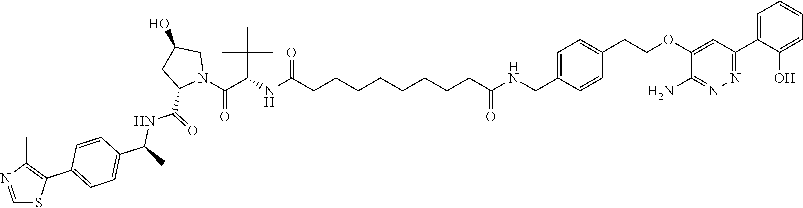 [SMILES: Cc1ncsc1-c1ccc([C@H](C)NC(=O)[C@@H]2C[C@@H](O)CN2C(=O)[C@@H](NC(=O)CCCCCCCCC(=O)NCc2ccc(CCOc3cc(-c4ccccc4O)nnc3N)cc2)C(C)(C)C)cc1] |
| 258 | 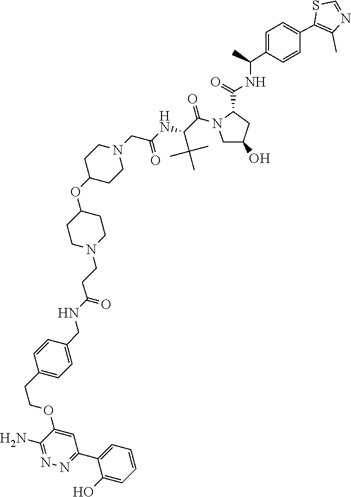 [SMILES: Cc1ncsc1-c1ccc([C@H](C)NC(=O)[C@@H]2C[C@@H](O)CN2C(=O)[C@@H](NC(=O)CN2CCC(OC3CCN(CCC(=O)NCc4ccc(CCOc5cc(-c6ccccc6O)nnc5N)cc4)CC3)CC2)C(C)(C)C)cc1] |
| 259 | 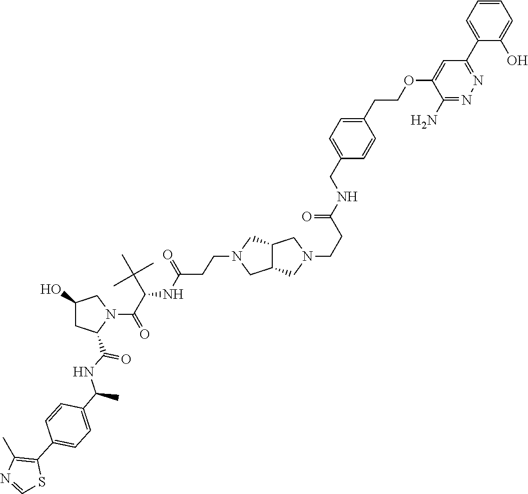 [SMILES: Cc1ncsc1-c1ccc([C@H](C)NC(=O)[C@@H]2C[C@@H](O)CN2C(=O)[C@@H](NC(=O)CCN2C[C@H]3CN(CCC(=O)NCc4ccc(CCOc5cc(-c6ccccc6O)nnc5N)cc4)C[C@H]3C2)C(C)(C)C)cc1] |
| 260 | 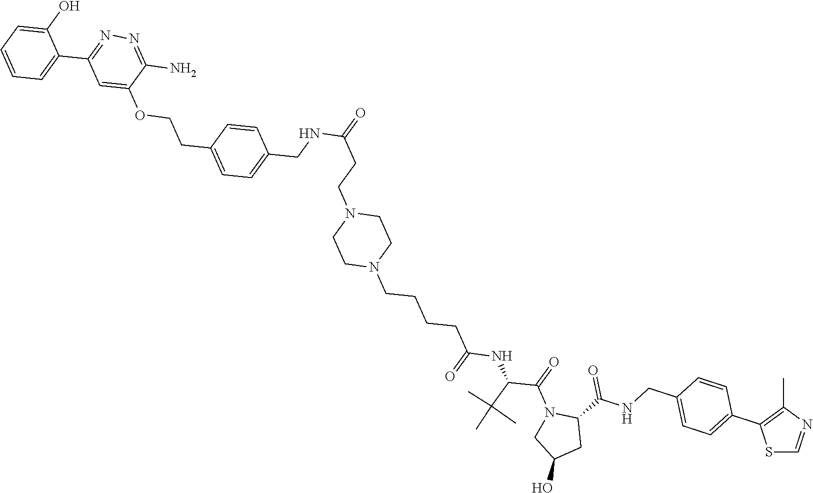 [SMILES: Cc1ncsc1-c1ccc(CNC(=O)[C@@H]2C[C@@H](O)CN2C(=O)[C@@H](NC(=O)CCCCN2CCN(CCC(=O)NCc3ccc(CCOc4cc(-c5ccccc5O)nnc4N)cc3)CC2)C(C)(C)C)cc1] |
| 261 | 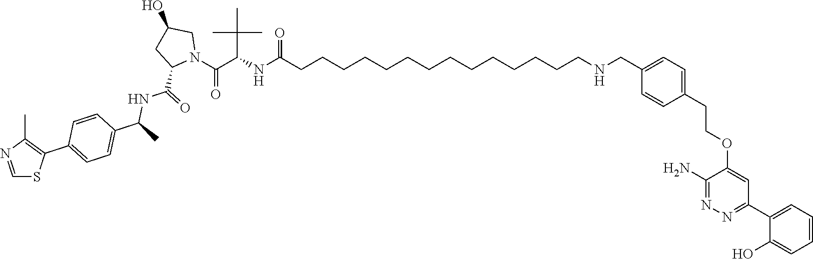 [SMILES: Cc1ncsc1-c1ccc([C@H](C)NC(=O)[C@@H]2C[C@@H](O)CN2C(=O)[C@@H](NC(=O)CCCCCCCCCCCCCCNCc2ccc(CCOc3cc(-c4ccccc4O)nnc3N)cc2)C(C)(C)C)cc1] |
| 262 | 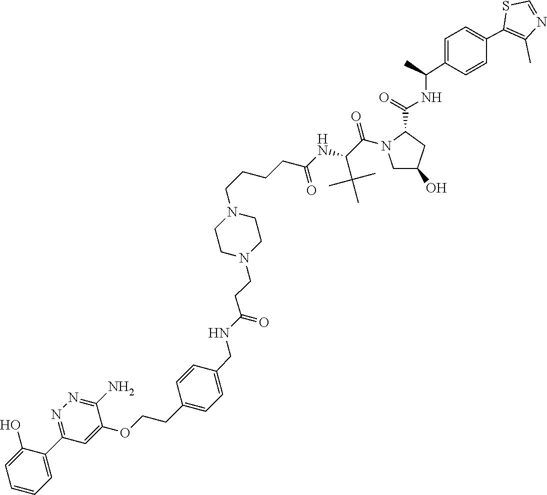 [SMILES: Cc1ncsc1-c1ccc([C@H](C)NC(=O)[C@@H]2C[C@@H](O)CN2C(=O)[C@@H](NC(=O)CCCCN2CCN(CCC(=O)NCc3ccc(CCOc4cc(-c5ccccc5O)nnc4N)cc3)CC2)C(C)(C)C)cc1] |
| 263 | 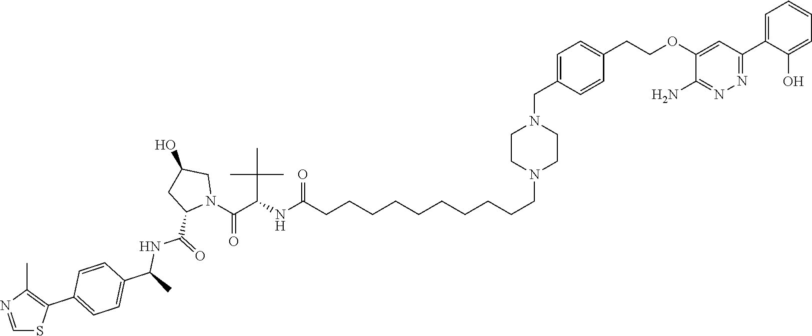 [SMILES: Cc1ncsc1-c1ccc([C@H](C)NC(=O)[C@@H]2C[C@@H](O)CN2C(=O)[C@@H](NC(=O)CCCCCCCCCCN2CCN(Cc3ccc(CCOc4cc(-c5ccccc5O)nnc4N)cc3)CC2)C(C)(C)C)cc1] |
| 264 | 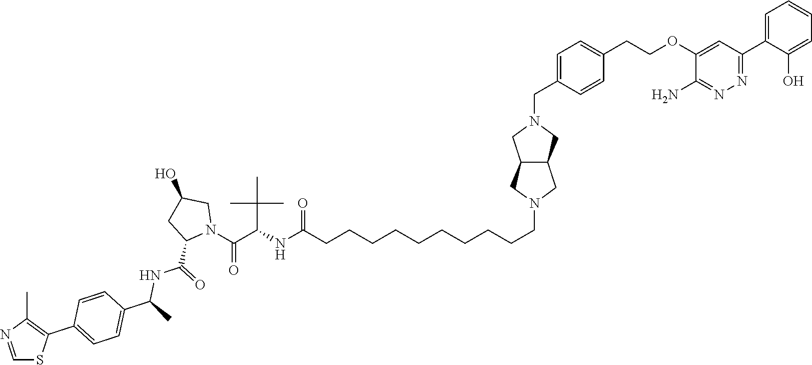 [SMILES: Cc1ncsc1-c1ccc([C@H](C)NC(=O)[C@@H]2C[C@@H](O)CN2C(=O)[C@@H](NC(=O)CCCCCCCCCCN2C[C@@H]3CN(Cc4ccc(CCOc5cc(-c6ccccc6O)nnc5N)cc4)C[C@@H]3C2)C(C)(C)C)cc1] |
| 265 | 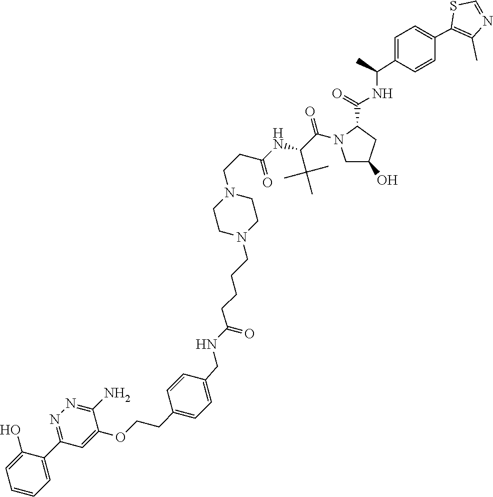 [SMILES: Cc1ncsc1-c1ccc([C@H](C)NC(=O)[C@@H]2C[C@@H](O)CN2C(=O)[C@@H](NC(=O)CCN2CCN(CCCCC(=O)NCc3ccc(CCOc4cc(-c5ccccc5O)nnc4N)cc3)CC2)C(C)(C)C)cc1] |
| 266 | 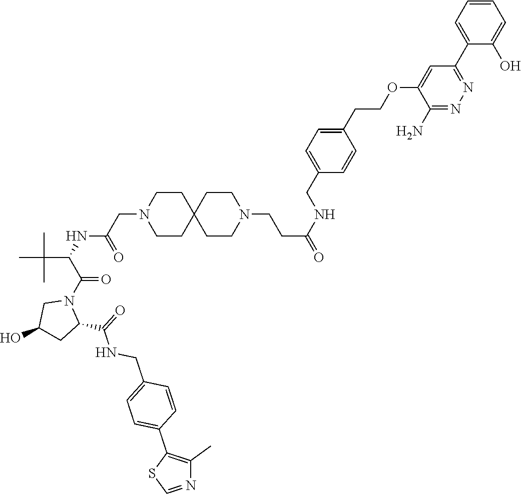 [SMILES: Cc1ncsc1-c1ccc(CNC(=O)[C@@H]2C[C@@H](O)CN2C(=O)[C@@H](NC(=O)CN2CCC3(CCN(CCC(=O)NCc4ccc(CCOc5cc(-c6ccccc6O)nnc5N)cc4)CC3)CC2)C(C)(C)C)cc1] |
| 267 | 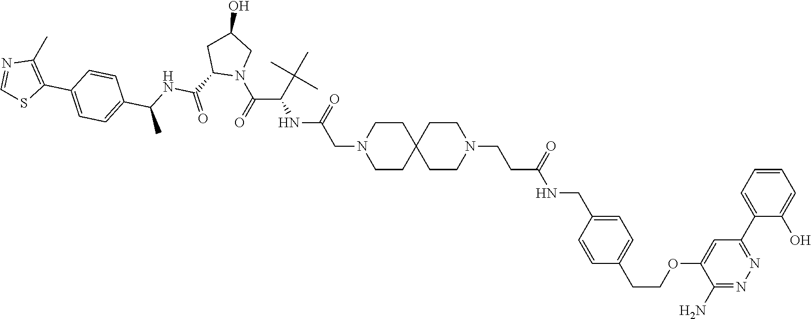 [SMILES: Cc1ncsc1-c1ccc([C@H](C)NC(=O)[C@@H]2C[C@@H](O)CN2C(=O)[C@@H](NC(=O)CN2CCC3(CCN(CCC(=O)NCc4ccc(CCOc5cc(-c6ccccc6O)nnc5N)cc4)CC3)CC2)C(C)(C)C)cc1] |

In an aspect, the invention features a pharmaceutical composition comprising any of the foregoing compounds and a pharmaceutically acceptable excipient.

In another aspect, the invention features a method of decreasing the activity of a BAF complex in a cell, the method involving contacting the cell with an effective amount of any of the foregoing compounds or a pharmaceutical composition thereof.

In some embodiments, the cell is a cancer cell.

In another aspect, the invention features a method of treating a BAF complex-related disorder in a subject in need thereof, the method involving administering to the subject an effective amount of any of the foregoing compounds (e.g., a BRM/BRG1 dual inhibitor compound or a BRM-selective compound) or a pharmaceutical composition thereof.

In some embodiments, the BAF complex-related disorder is cancer.

In a further aspect, the invention features a method of inhibiting BRM, the method involving contacting a cell with an effective amount of any of the foregoing compounds (e.g., a BRM/BRG1 dual inhibitor compound or a BRM-selective compound) or a pharmaceutical composition thereof.

In some embodiments, the cell is a cancer cell.

In another aspect, the invention features a method of inhibiting BRG1, the method involving contacting the cell with an effective amount of any of the foregoing compounds or a pharmaceutical composition thereof.

In some embodiments, the cell is a cancer cell.

In a further aspect, the invention features a method of inhibiting BRM and BRG1, the method involving contacting the cell with an effective amount of any of the foregoing compounds or a pharmaceutical composition thereof.

In some embodiments, the cell is a cancer cell.

In another aspect, the invention features a method of treating a disorder related to a BRG1 loss of function mutation in a subject in need thereof, the method involving administering to the subject an effective amount of any of the foregoing compounds (e.g., a BRM/BRG1 dual inhibitor compound or a BRM-selective compound) or a pharmaceutical composition thereof.

In some embodiments, the disorder related to a BRG1 loss of function mutation is cancer. In other embodiments, the subject is determined to have a BRG1 loss of function disorder, for example, is determined to have a BRG1 loss of function cancer (for example, the cancer has been determined to include cancer cells with loss of BRG1 function).

In another aspect, the invention features a method of inducing apoptosis in a cell, the method involving contacting the cell with an effective amount of any of the foregoing compounds (e.g., a BRM/BRG1 dual inhibitor compound or a BRM-selective compound) or a pharmaceutical composition thereof.

In some embodiments, the cell is a cancer cell.

In a further aspect, the invention features a method of treating cancer in a subject in need thereof, the method including administering to the subject an effective amount of any of the foregoing compounds (e.g., a BRM/BRG1 dual inhibitor compound or a BRM-selective compound) or a pharmaceutical composition thereof.

In some embodiments of any of the foregoing methods, the cancer is non-small cell lung cancer, colorectal cancer, bladder cancer, cancer of unknown primary, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.

In some embodiments of any of the foregoing methods, the cancer is non-small cell lung cancer, colorectal cancer, bladder cancer, cancer of unknown primary, glioma, breast cancer, melanoma, non-melanoma skin cancer, endometrial cancer, or penile cancer.

In some embodiments of any of the foregoing methods, the cancer is a drug resistant cancer or has failed to respond to a prior therapy (e.g., vemurafenib, dacarbazine, a CTLA4 inhibitor, a PD1 inhibitor, interferon therapy, a BRAF inhibitor, a MEK inhibitor, radiotherapy, temozolomide, irinotecan, a CAR-T therapy, Herceptin®, Perjeta®, tamoxifen, Xeloda®, docetaxol, platinum agents such as carboplatin, taxanes such as paclitaxel and docetaxel, ALK inhibitors, MET inhibitors, Alimta®, Abraxane®, Adriamycin®, gemcitabine, Avastin®, Halaven®, neratinib, a PARP inhibitor, ARN810, an mTOR inhibitor, topotecan, Gemzar®, a VEGFR2 inhibitor, a folate receptor antagonist, demcizumab, fosbretabulin, or a PDL1 inhibitor).

In some embodiments of any of the foregoing methods, the cancer has or has been determined to have BRG1 mutations. In some embodiments of any of the foregoing methods, the BRG1 mutations are homozygous. In some embodiments of any of the foregoing methods, the cancer does not have, or has been determined not to have, an epidermal growth factor receptor (EGFR) mutation. In some embodiments of any of the foregoing methods, the cancer does not have, or has been determined not to have, an anaplastic lymphoma kinase (ALK) driver mutation. In some embodiments of any of the foregoing methods, the cancer has, or has been determined to have, a KRAS mutation. In some embodiments of any of the foregoing methods, the BRG1 mutation is in the ATPase catalytic domain of the protein. In some embodiments of any of the foregoing methods, the BRG1 mutation is a deletion at the C-terminus of BRG1.

In another aspect, the disclosure provides a method treating a disorder related to BAF (e.g., cancer or viral infections) in a subject in need thereof. This method includes contacting a cell with an effective amount of any of the foregoing compounds (e.g., a BRM/BRG1 dual inhibitor compound or a BRM-selective compound), or pharmaceutically acceptable salts thereof, or any of the foregoing pharmaceutical compositions. In some embodiments, the disorder is a viral infection is an infection with a virus of the Retroviridae family such as the lentiviruses (e.g., Human immunodeficiency virus (HIV) and deltaretroviruses (e.g., human T cell leukemia virus I (HTLV-I), human T cell leukemia virus II (HTLV-II)), Hepadnaviridae family (e.g., hepatitis B virus (HBV)), Flaviviridae family (e.g., hepatitis C virus (HCV)), Adenoviridae family (e.g., Human Adenovirus), Herpesviridae family (e.g., Human cytomegalovirus (HCMV), Epstein-Barr virus, herpes simplex virus 1 (HSV-1), herpes simplex virus 2 (HSV-2), human herpesvirus 6 (HHV-6), Herpesvitus K*, CMV, varicella-zoster virus), Papillomaviridae family (e.g., Human Papillomavirus (HPV, HPV E1)), Parvoviridae family (e.g., Parvovirus B19), Polyomaviridae family (e.g., JC virus and BK virus), Paramyxoviridae family (e.g., Measles virus), Togaviridae family (e.g., Rubella virus). In some embodiments, the disorder is Coffin Siris, Neurofibromatosis (e.g., NF-1, NF-2, or Schwannomatosis), or Multiple Meningioma.

In another aspect, the disclosure provides a method for treating a viral infection in a subject in need thereof. This method includes administering to the subject an effective amount of any of the foregoing compounds (e.g., a BRM/BRG1 dual inhibitor compound or a BRM-selective compound), or pharmaceutically acceptable salts thereof, or any of the foregoing pharmaceutical compositions. In some embodiments, the viral infection is an infection with a virus of the Retroviridae family such as the lentiviruses (e.g., Human immunodeficiency virus (HIV) and deltaretroviruses (e.g., human T cell leukemia virus I (HTLV-I), human T cell leukemia virus II (HTLV-II)), Hepadnaviridae family (e.g., hepatitis B virus (HBV)), Flaviviridae family (e.g., hepatitis C virus (HCV)), Adenoviridae family (e.g., Human Adenovirus), Herpesviridae family (e.g., Human cytomegalovirus (HCMV), Epstein-Barr virus, herpes simplex virus 1 (HSV-1), herpes simplex virus 2 (HSV-2), human herpesvirus 6 (HHV-6), Herpesvitus K*, CMV, varicella-zoster virus), Papillomaviridae family (e.g., Human Papillomavirus (HPV, HPV E1)), Parvoviridae family (e.g., Parvovirus B19), Polyomaviridae family (e.g., JC virus and BK virus), Paramyxoviridae family (e.g., Measles virus), or Togaviridae family (e.g., Rubella virus).

In some embodiments of any of the foregoing aspects, the compound is a BRM-selective compound. In some embodiments, the BRM-selective compound inhibits the level and/or activity of BRM at least 10-fold greater than the compound inhibits the level and/or activity of BRG1 and/or the compound binds to BRM at least 10-fold greater than the compound binds to BRG1. For example, in some embodiments, a BRM-selective compound has an IC₅₀ or IP₅₀ that is at least 10-fold lower than the IC₅₀ or IP₅₀ against BRG1. In some embodiments of any of the foregoing aspects, the compound is a BRM/BRG1 dual inhibitor compound. In some embodiments, the BRM/BRG1 dual inhibitor compound has similar activity against both BRM and BRG1 (e.g., the activity of the compound against BRM and BRG1 with within 10-fold (e.g., less than 5-fold, less than 2-fold). In some embodiments, the activity of the BRM/BRG1 dual inhibitor compound is greater against BRM. In some embodiments, the activity of the BRM/BRG1 dual inhibitor compound is greater against BRG1. For example, in some embodiments, a BRM/BRG1 dual inhibitor compound has an IC₅₀ or IP₅₀ against BRM that is within 10-fold of the IC₅₀ or IP₅₀ against BRG1.

In another aspect, the invention features a method of treating melanoma, prostate cancer, breast cancer, bone cancer, renal cell carcinoma, or a hematologic cancer in a subject in need thereof, the method including administering to the subject an effective amount of any of the foregoing compounds or pharmaceutical compositions thereof.

In another aspect, the invention features a method of reducing tumor growth of melanoma, prostate cancer, breast cancer, bone cancer, renal cell carcinoma, or a hematologic cancer in a subject in need thereof, the method including administering to the subject an effective amount of any of the foregoing compounds or pharmaceutical compositions thereof.

In another aspect, the invention features a method of suppressing metastatic progression of melanoma, prostate cancer, breast cancer, bone cancer, renal cell carcinoma, or a hematologic cancer in a subject, the method including administering an effective amount of any of the foregoing compounds or pharmaceutical compositions thereof.

In another aspect, the invention features a method of suppressing metastatic colonization of melanoma, prostate cancer, breast cancer, bone cancer, renal cell carcinoma, or a hematologic cancer in a subject, the method including administering an effective amount of any of the foregoing compounds or pharmaceutical compositions thereof.

In another aspect, the invention features a method of reducing the level and/or activity of BRG1 and/or BRM in a melanoma, prostate cancer, breast cancer, bone cancer, renal cell carcinoma, or hematologic cancer cell, the method including contacting the cell with an effective amount of any of the foregoing compounds or pharmaceutical compositions thereof.

In some embodiments of any of the above aspects, the melanoma, prostate cancer, breast cancer, bone cancer, renal cell carcinoma, or hematologic cell is in a subject.

In some embodiments of any of the above aspects, the effective amount of the compound reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference. In some embodiments, the effective amount of the compound that reduces the level and/or activity of BRG1 by at least 50% (e.g., 55%, 60%, 65%, 70%, 75%, 80%, 85%, 90%, or 95%) as compared to a reference. In some embodiments, the effective amount of the compound that reduces the level and/or activity of BRG1 by at least 90% (e.g., 91%, 92%, 93%, 94%, 95%, 96%, 97%, 98%, or 99%).

In some embodiments, the effective amount of the compound reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 12 hours (e.g., 14 hours, 16 hours, 18 hours, 20 hours, 22 hours, 24 hours, 30 hours, 36 hours, 48 hours, 72 hours, or more). In some embodiments, the effective amount of the compound that reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 4 days (e.g., 5 days, 6 days, 7 days, 14 days, 28 days, or more).

In some embodiments of any of the above aspects, the effective amount of the compound reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference. In some embodiments, the effective amount of the compound that reduces the level and/or activity of BRM by at least 50% (e.g., 55%, 60%, 65%, 70%, 75%, 80%, 85%, 90%, or 95%) as compared to a reference. In some embodiments, the effective amount of the compound that reduces the level and/or activity of BRM by at least 90% (e.g., 91%, 92%, 93%, 94%, 95%, 96%, 97%, 98%, or 99%).

In some embodiments, the effective amount of the compound reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 12 hours (e.g., 14 hours, 16 hours, 18 hours, 20 hours, 22 hours, 24 hours, 30 hours, 36 hours, 48 hours, 72 hours, or more). In some embodiments, the effective amount of the compound that reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 4 days (e.g., 5 days, 6 days, 7 days, 14 days, 28 days, or more).

In some embodiments, the subject has cancer. In some embodiments, the cancer expresses BRG1 and/or BRM protein and/or the cell or subject has been identified as expressing BRG1 and/or BRM. In some embodiments, the cancer expresses BRG1 protein and/or the cell or subject has been identified as expressing BRG1. In some embodiments, the cancer expresses BRM protein and/or the cell or subject has been identified as expressing BRM. In some embodiments, the cancer is melanoma (e.g., uveal melanoma, mucosal melanoma, or cutaneous melanoma). In some embodiments, the cancer is prostate cancer. In some embodiments, the cancer is a hematologic cancer, e.g., multiple myeloma, large cell lymphoma, acute T-cell leukemia, acute myeloid leukemia, myelodysplastic syndrome, immunoglobulin A lambda myeloma, diffuse mixed histiocytic and lymphocytic lymphoma, B-cell lymphoma, acute lymphoblastic leukemia (e.g., T-cell acute lymphoblastic leukemia or B-cell acute lymphoblastic leukemia), diffuse large cell lymphoma, or non-Hodgkin's lymphoma. In some embodiments, the cancer is breast cancer (e.g., an ER positive breast cancer, an ER negative breast cancer, triple positive breast cancer, or triple negative breast cancer). In some embodiments, the cancer is a bone cancer (e.g., Ewing's sarcoma). In some embodiments, the cancer is a renal cell carcinoma (e.g., a Microphthalmia Transcription Factor (MITF) family translocation renal cell carcinoma (tRCC)). In some embodiments, the cancer is metastatic (e.g., the cancer has spread to the liver). The metastatic cancer can include cells exhibiting migration and/or invasion of migrating cells and/or include cells exhibiting endothelial recruitment and/or angiogenesis. In other embodiments, the migrating cancer is a cell migration cancer. In still other embodiments, the cell migration cancer is a non-metastatic cell migration cancer. The metastatic cancer can be a cancer spread via seeding the surface of the peritoneal, pleural, pericardial, or subarachnoid spaces. Alternatively, the metastatic cancer can be a cancer spread via the lymphatic system, or a cancer spread hematogenously. In some embodiments, the effective amount of an agent that reduces the level and/or activity of BRG1 and/or BRM is an amount effective to inhibit metastatic colonization of the cancer to the liver.

In some embodiments the cancer harbors a mutation in GNAQ. In some embodiments the cancer harbors a mutation in GNA11. In some embodiments the cancer harbors a mutation in PLCB4. In some embodiments the cancer harbors a mutation in CYSLTR2. In some embodiments the cancer harbors a mutation in BAP1. In some embodiments the cancer harbors a mutation in SF3B1. In some embodiments the cancer harbors a mutation in EIF1AX. In some embodiments the cancer harbors a TFE3 translocation. In some embodiments the cancer harbors a TFEB translocation. In some embodiments the cancer harbors a MITF translocation. In some embodiments the cancer harbors an EZH2 mutation. In some embodiments the cancer harbors a SUZ12 mutation. In some embodiments the cancer harbors an EED mutation.

In some embodiments, the method further includes administering to the subject or contacting the cell with an anticancer therapy, e.g., a chemotherapeutic or cytotoxic agent, immunotherapy, surgery, radiotherapy, thermotherapy, or photocoagulation. In some embodiments, the anticancer therapy is a chemotherapeutic or cytotoxic agent, e.g., an antimetabolite, antimitotic, antitumor antibiotic, asparagine-specific enzyme, bisphosphonates, antineoplastic, alkylating agent, DNA-Repair enzyme inhibitor, histone deacetylase inhibitor, corticosteroid, demethylating agent, immunomodulatory, janus-associated kinase inhibitor, phosphinositide 3-kinase inhibitor, proteasome inhibitor, or tyrosine kinase inhibitor.

In some embodiments, the compound of the invention is used in combination with another anti-cancer therapy used for the treatment of uveal melanoma such as surgery, a MEK inhibitor, and/or a PKC inhibitor. For example, in some embodiments, the method further comprises performing surgery prior to, subsequent to, or at the same time as administration of the compound of the invention. In some embodiments, the method further comprises administration of a MEK inhibitor and/or a PKC inhibitor prior to, subsequent to, or at the same time as administration of the compound of the invention.

In some embodiments, the anticancer therapy and the compound of the invention are administered within 28 days of each other and each in an amount that together are effective to treat the subject.

In some embodiments, the subject or cancer has and/or has been identified as having a BRG1 loss of function mutation. In some embodiments, the subject or cancer has and/or has been identified as having a BRM loss of function mutation.

In some embodiments, the cancer is resistant to one or more chemotherapeutic or cytotoxic agents (e.g., the cancer has been determined to be resistant to chemotherapeutic or cytotoxic agents such as by genetic markers, or is likely to be resistant, to chemotherapeutic or cytotoxic agents such as a cancer that has failed to respond to a chemotherapeutic or cytotoxic agent). In some embodiments, the cancer has failed to respond to one or more chemotherapeutic or cytotoxic agents. In some embodiments, the cancer is resistant or has failed to respond to dacarbazine, temozolomide, cisplatin, treosulfan, fotemustine, IMCgp100, a CTLA-4 inhibitor (e.g., ipilimumab), a PD-1 inhibitor (e.g., Nivolumab or pembrolizumab), a PD-L1 inhibitor (e.g., atezolizumab, avelumab, or durvalumab), a mitogen-activated protein kinase (MEK) inhibitor (e.g., selumetinib, binimetinib, or tametinib), and/or a protein kinase C (PKC) inhibitor (e.g., sotrastaurin or IDE196).

In some embodiments, the cancer is resistant to or failed to respond to a previously administered therapeutic used for the treatment of uveal melanoma such as a MEK inhibitor or PKC inhibitor. For example, in some embodiments, the cancer is resistant to or failed to respond to a mitogen-activated protein kinase (MEK) inhibitor (e.g., selumetinib, binimetinib, or tametinib), and/or a protein kinase C (PKC) inhibitor (e.g., sotrastaurin or IDE196).

### Chemical Terms

The terminology employed herein is for the purpose of describing particular embodiments and is not intended to be limiting.

For any of the following chemical definitions, a number following an atomic symbol indicates that total number of atoms of that element that are present in a particular chemical moiety. As will be understood, other atoms, such as H atoms, or substituent groups, as described herein, may be present, as necessary, to satisfy the valences of the atoms. For example, an unsubstituted C₂ alkyl group has the formula —CH₂CH₃. When used with the groups defined herein, a reference to the number of carbon atoms includes the divalent carbon in acetal and ketal groups but does not include the carbonyl carbon in acyl, ester, carbonate, or carbamate groups. A reference to the number of oxygen, nitrogen, or sulfur atoms in a heteroaryl group only includes those atoms that form a part of a heterocyclic ring.

The term “acyl,” as used herein, represents a H or an alkyl group that is attached to a parent molecular group through a carbonyl group, as defined herein, and is exemplified by formyl (i.e., a carboxaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, 1 to 6 carbon atoms, or 1 to 3 carbon atoms).

An alkylene is a divalent alkyl group. The term “alkenyl,” as used herein, alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6 carbon atoms, or 2 carbon atoms).

The term “alkynyl,” as used herein, alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6 carbon atoms, or 2 carbon atoms).

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the invention can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁-C₆ alkyl C₆-C₁₀ aryl, C₁-C₁₀ alkyl C₆-C₁₀ aryl, or C₁-C₂₀ alkyl C₆-C₁₀ aryl), such as, benzyl and phenethyl. In some embodiments, the alkyl and the aryl each can be further substituted with 1, 2, 3, or 4 substituent groups as defined herein for the respective groups.

The term “azido,” as used herein, represents a —N₃ group.

The term “bridged polycycloalkyl,” as used herein, refers to a bridged polycyclic group of 5 to 20 carbons, containing from 1 to 3 bridges.

The term “cyano,” as used herein, represents a —CN group.

The term “carbocyclyl,” as used herein, refers to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, and monovalent mono- or polycarbocyclic radical of 3 to 10, preferably 3 to 6 carbon atoms. This term is further exemplified by radicals such as cyclopropyl, cyclobutyl, cyclopentyl, cyclohexyl, cycloheptyl, norbornyl, and adamantyl.

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group. The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group. The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups.

Examples of heteroalkynyl groups are an “alkynoxy” which, as used herein, refers alkynyl-O—. A heteroalkynylene is a divalent heteroalkynyl group.

The term “heteroaryl,” as used herein, refers to a mono- or polycyclic radical of 5 to 12 atoms having at least one aromatic ring containing 1, 2, or 3 ring atoms selected from nitrogen, oxygen, and sulfur, with the remaining ring atoms being carbon. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

The term “heterocyclyl,” as used herein, refers a mono- or polycyclic radical having 3 to 12 atoms having at least one ring containing 1, 2, 3, or 4 ring atoms selected from N, O or S, wherein no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

The term “hydroxyalkyl,” as used herein, represents alkyl group substituted with an —OH group.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3rd Edition (John Wiley & Sons, New York, 1999). N-protecting groups include, but are not limited to, acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L, or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-20 dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halo (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, oxo, or thiol. In some embodiments, substituents include: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halo (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbents or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent. Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

In this application, unless otherwise clear from context, (i) the term “a” may be understood to mean “at least one”; (ii) the term “or” may be understood to mean “and/or”; and (iii) the terms “comprising” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “BAF complex” refers to the BRG1- or HRBM-associated factors complex in a human cell.

As used herein, the term “BAF complex-related disorder” refers to a disorder that is caused or affected by the level of activity of a BAF complex.

As used herein, the term “BRG1 loss of function mutation” refers to a mutation in BRG1 that leads to the protein having diminished activity (e.g., at least 1% reduction in BRG1 activity, for example 2%, 5%, 10%, 25%, 50%, or 100% reduction in BRG1 activity). Exemplary BRG1 loss of function mutations include, but are not limited to, a homozygous BRG1 mutation and a deletion at the C-terminus of BRG1.

As used herein, the term “BRG1 loss of function disorder” refers to a disorder (e.g., cancer) that exhibits a reduction in BRG1 activity (e.g., at least 1% reduction in BRG1 activity, for example 2%, 5%, 10%, 25%, 50%, or 100% reduction in BRG1 activity).

The term “cancer” refers to a condition caused by the proliferation of malignant neoplastic cells, such as tumors, neoplasms, carcinomas, sarcomas, leukemias, and lymphomas.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

By “determining the level” of a protein or RNA is meant the detection of a protein or an RNA, by methods known in the art, either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure RNA levels are known in the art and include, but are not limited to, quantitative polymerase chain reaction (qPCR) and Northern blot analyses.

By “decreasing the activity of a BAF complex” is meant decreasing the level of an activity related to a BAF complex, or a related downstream effect. A non-limiting example of decreasing an activity of a BAF complex is Sox2 activation. The activity level of a BAF complex may be measured using any method known in the art, e.g., the methods described in Kadoch et al. Cell, 2013, 153, 71-85, the methods of which are herein incorporated by reference.

As used herein, the term “degrader” refers to a small molecule compound including a degradation moiety, wherein the compound interacts with a protein (e.g., BRG1 and/or BRM) in a way which results in degradation of the protein, e.g., binding of the compound results in at least 5% reduction of the level of the protein, e.g., in a cell or subject.

As used herein, the term “degradation moiety” refers to a moiety whose binding results in degradation of a protein, e.g., BRG1 and/or BRM. In one example, the moiety binds to a protease or a ubiquitin ligase that metabolizes the protein, e.g., BRG1 and/or BRM.

By “modulating the activity of a BAF complex,” is meant altering the level of an activity related to a BAF complex (e.g., GBAF), or a related downstream effect. The activity level of a BAF complex may be measured using any method known in the art, e.g., the methods described in Kadoch et al, Cell 153:71-85 (2013), the methods of which are herein incorporated by reference.

By “reducing the activity of BRG1 and/or BRM,” is meant decreasing the level of an activity related to an BRG1 and/or BRM, or a related downstream effect. A non-limiting example of inhibition of an activity of BRG1 and/or BRM is decreasing the level of a BAF complex in a cell. The activity level of BRG1 and/or BRM may be measured using any method known in the art. In some embodiments, an agent which reduces the activity of BRG1 and/or BRM is a small molecule BRG1 and/or BRM degrader.

By “reducing the level of BRG1 and/or BRM,” is meant decreasing the level of BRG1 and/or BRM in a cell or subject. The level of BRG1 and/or BRM may be measured using any method known in the art.

By “level” is meant a level of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or mRNA in a sample.

As used herein, the term “inhibiting BRM” refers to blocking or reducing the level or activity of the ATPase catalytic binding domain or the bromodomain of the protein. BRM inhibition may be determined using methods known in the art, e.g., a BRM ATPase assay, a Nano DSF assay, or a BRM Luciferase cell assay.

The term “pharmaceutical composition,” as used herein, represents a composition containing a compound described herein formulated with a pharmaceutically acceptable excipient and appropriate for administration to a mammal, for example a human. Typically, a pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration.

As used herein, the term “pharmaceutically acceptable salt” means any pharmaceutically acceptable salt of a compound, for example, any compound of Formula I or II. Pharmaceutically acceptable salts of any of the compounds described herein may include those that are within the scope of sound medical judgment, suitable for use in contact with the tissues of humans and animals without undue toxicity, irritation, allergic response and are commensurate with a reasonable benefit/risk ratio.

Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

By a “reference” is meant any useful reference used to compare protein or RNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound of the invention; a sample from a subject that has been treated by a compound of the invention; or a sample of a purified protein or RNA (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound of the invention. In preferred embodiments, the reference sample, standard, or level is matched to the sample subject sample by at least one of the following criteria: age, weight, sex, disease stage, and overall health. A standard curve of levels of a purified protein or RNA, e.g., any described herein, within the normal reference range can also be used as a reference.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean therapeutic treatment or any measures whose object is to slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total); an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment. Compounds of the invention may also be used to “prophylactically treat” or “prevent” a disorder, for example, in a subject at increased risk of developing the disorder.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

The details of one or more embodiments of the invention are set forth in the description below. Other features, objects, and advantages of the invention will be apparent from the description and from the claims.

### DETAILED DESCRIPTION

The present disclosure features compounds useful for the inhibition of BRG1 and/or BRM. These compounds may be used to modulate the activity of a BAF complex, for example, for the treatment of a BAF-related disorder, such as cancer. Exemplary compounds described herein include compounds having a structure according to Formula I or II, or a pharmaceutically acceptable salt thereof.

Formula I:

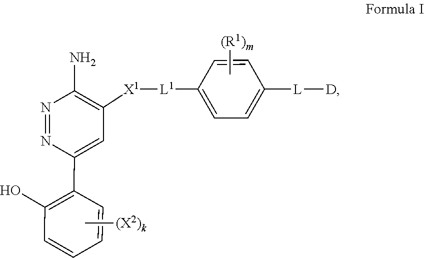

where

X¹ is O or NR²;

each X² is independently a halogen;

k is 0, 1, 2, 3, or 4;

m is 0, 1, 2, 3, or 4;

R¹ is halo or optionally substituted C₁-C₆ alkyl;

R² is H or optionally substituted C₁-C₆ alkyl;

L¹ is optionally substituted C₁-C₆ alkylene;

L is a linker including the structure of

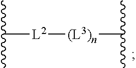

n is 0, 1, 2, or 3;

L² is optionally substituted C₁-C₆ alkylene, optionally substituted C₁-C₂₀ heteroalkylene, or optionally substituted C₂-C₉ heterocyclylene;

each L³ is, independently, —O—, optionally substituted C₁-C₂₀ heteroalkylene, optionally substituted C₃-C₁₀ carbocyclylene, optionally substituted C₃-C₁₀ carbocyclylene-C₁-C₂₀ alkylene, optionally substituted C₂-C₉ heterocyclylene, or optionally substituted C₂-C₉ heterocyclylene-C₁-C₂₀ alkylene; and

D is a degradation moiety.

Formula II:

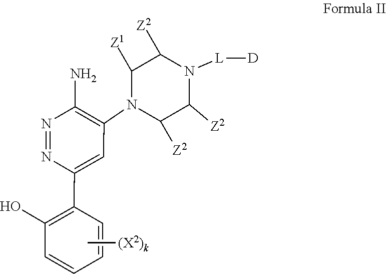

where

one Z¹ and one Z² combine to form an optionally substituted C₁-C₄ alkylene, and the remaining Z¹ and Z² are each hydrogen;

each X² is independently a halogen;

k is 0, 1, 2, 3, or 4;

L is a linker having the structure of

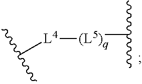

q is 0, 1, 2, 3, or 4;

L⁴ is optionally substituted C₁-C₆ alkylene, optionally substituted C₁-C₂₀ heteroalkylene, or optionally substituted C₂-C₉ heteroarylene;

each L⁵ is independently —O—, optionally substituted C₁-C₆ alkylene, optionally substituted C₁-C₂₀ heteroalkylene, optionally substituted C₃-C₁₀ carbocyclylene, optionally substituted C₃-C₁₀ carbocyclylene-C₁-C₆ alkylene, optionally substituted C₂-C₉ heterocyclylene, or C₂-C₉ heterocyclylene-C₁-C₂₀ alkylene; and

D is a degradation moiety.

In some embodiments, the compound has the structure of any one of compounds 1-75 in Table 1 or 76-104 in Table 2, or pharmaceutically acceptable salt thereof.

Other embodiments, as well as exemplary methods for the synthesis of production of these compounds, are described herein.

### Pharmaceutical Uses

The compounds described herein are useful in the methods of the invention and, while not bound by theory, are believed to exert their ability to modulate the level, status, and/or activity of a BAF complex, i.e., by inhibiting the activity of the BRG1 and/or BRM proteins within the BAF complex in a mammal. BAF complex-related disorders include, but are not limited to, BRG1 loss of function mutation-related disorders.

An aspect of the present invention relates to methods of treating disorders related to BRG1 loss of function mutations such as cancer (e.g., non-small cell lung cancer, colorectal cancer, bladder cancer, cancer of unknown primary, glioma, breast cancer, melanoma, non-melanoma skin cancer, endometrial cancer, or penile cancer) in a subject in need thereof. In some embodiments, the compound is administered in an amount and for a time effective to result in one or more (e.g., two or more, three or more, four or more) of: (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, (i) increased progression free survival of subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound of the invention. An increase in average survival time of a population may also be measured, for example, by calculating for a population the average length of survival following completion of a first round of treatment with a pharmaceutically acceptable salt of the invention.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of the invention. A decrease in the mortality rate of a population may also be measured, for example, by calculating for a population the average number of disease-related deaths per unit time following completion of a first round of treatment with a pharmaceutically acceptable salt of the invention.

Exemplary cancers that may be treated by the invention include, but are not limited to, non-small cell lung cancer, small-cell lung cancer, colorectal cancer, bladder cancer, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer and penile cancer.

### Combination Formulations and Uses Thereof

The compounds of the invention can be combined with one or more therapeutic agents. In particular, the therapeutic agent can be one that treats or prophylactically treats any cancer described herein.

### Combination Therapies

A compound of the invention can be used alone or in combination with an additional therapeutic agent, e.g., other agents that treat cancer or symptoms associated therewith, or in combination with other types of treatment to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammaII and calicheamicin omegaII (see, e.g., Agnew, Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, Adriamycin® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), ABraxane®, cremophor-free, albumin-engineered nanoparticle formulation of paclitaxel (American Pharmaceutical Partners, Schaumberg, III.), and Taxotere® doxetaxel (Rhone-Poulenc Rorer, Antony, France); chloranbucil; Gemzar® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al. (1999) Proc ASCO 18:233a and Douillard et al. (2000) Lancet 355:1041-7.

In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (Avastin®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response or antagonizes an antigen important for cancer. Such agents include Rituxan (Rituximab); Zenapax (Daclizumab); Simulect (Basiliximab); Synagis (Palivizumab); Remicade (Infliximab); Herceptin (Trastuzumab); Mylotarg (Gemtuzumab ozogamicin); Campath (Alemtuzumab); Zevalin (Ibritumomab tiuxetan); Humira (Adalimumab); Xolair (Omalizumab); Bexxar (Tositumomab-I-131); Raptiva (Efalizumab); Erbitux (Cetuximab); Avastin (Bevacizumab); Tysabri (Natalizumab); Actemra (Tocilizumab); Vectibix (Panitumumab); Lucentis (Ranibizumab); Soliris (Eculizumab); Cimzia (Certolizumab pegol); Simponi (Golimumab); Ilaris (Canakinumab); Stelara (Ustekinumab); Arzerra (Ofatumumab); Prolia (Denosumab); Numax (Motavizumab); ABThrax (Raxibacumab); Benlysta (Belimumab); Yervoy (Ipilimumab); Adcetris (Brentuximab Vedotin); Perjeta (Pertuzumab); Kadcyla (Ado-trastuzumab emtansine); and Gazyva (Obinutuzumab). Also included are antibody-drug conjugates.

The second agent may be a therapeutic agent which is a non-drug treatment. For example, the second therapeutic agent is radiation therapy, cryotherapy, hyperthermia and/or surgical excision of tumor tissue.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab). In some embodiments, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011). In some embodiments, the inhibitor of checkpoint is an inhibitor (e.g., an inhibitory antibody or small molecule inhibitor) of PDL1 (e.g., MPDL3280A/RG7446; MEDI4736; MSB0010718C; BMS 936559). In some embodiments, the inhibitor of checkpoint is an inhibitor (e.g., an inhibitory antibody or Fc fusion or small molecule inhibitor) of PDL2 (e.g., a PDL2/Ig fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### Pharmaceutical Compositions

The compounds of the invention are preferably formulated into pharmaceutical compositions for administration to a mammal, preferably, a human, in a biologically compatible form suitable for administration in vivo. Accordingly, in an aspect, the present invention provides a pharmaceutical composition comprising a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard- or soft-shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers. A compound of the invention may also be administered parenterally. Solutions of a compound of the invention can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003, 20th ed.) and in The United States Pharmacopeia: The National Formulary (USP 24 NF19), published in 1999. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter. A compound described herein may be administered intratumorally, for example, as an intratumoral injection. Intratumoral injection is injection directly into the tumor vasculature and is specifically contemplated for discrete, solid, accessible tumors. Local, regional, or systemic administration also may be appropriate. A compound described herein may advantageously be contacted by administering an injection or multiple injections to the tumor, spaced for example, at approximately, 1 cm intervals. In the case of surgical intervention, the present invention may be used preoperatively, such as to render an inoperable tumor subject to resection. Continuous administration also may be applied where appropriate, for example, by implanting a catheter into a tumor or into tumor vasculature.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of, for example, between 0.05 mg and 3000 mg (measured as the solid form). Dose ranges include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered.

Alternatively, the dosage amount can be calculated using the body weight of the patient. For example, the dose of a compound, or pharmaceutical composition thereof, administered to a patient may range from 0.1-100 mg/kg (e.g., 0.25-25 mg/kg). In exemplary, non-limiting embodiments, the dose may range from 0.5-5.0 mg/kg (e.g., 0.5, 1.0, 1.5, 2.0, 2.5, 3.0, 3.5, 4.0, 4.5, or 5.0 mg/kg) or from 5.0-20 mg/kg (e.g., 5.5, 6.0, 6.5, 7.0, 7.5, 8.0, 8.5, 9.0, 9.5, 10, 11, 12, 13, 14, 15, 16, 17, 18, 19, or 20 mg/kg).

### EXAMPLES

The following abbreviations are used throughout the Examples below.
Ac acetyl
ACN or MeCN acetonitrile
AcOH acetic acid
Ac₂O acetic anhydride
aq. aqueous
Boc tert-butoxycarbonyl
Bu or n-Bu butyl
CDI 1,1′-carbonyldiimidazole
DCE or 1,2-DCE 1,2-dichloroethane
DCM dichloromethane
DIAD diisopropyl azodicarboxylate
DIPEA or DIEA N.N-diisopropylethylamine
DMAP 4-(dimethylamino)pyridine
DME 1,2-dimethoxyethane
DMF N.N-dimethylformamide
DMSO dimethyl sulfoxide
EA or EtOAc ethyl acetate
EDCI N-(3-dimethylaminopropyl)-N′-ethylcarbodiimide hydrochloride
equiv equivalents
Et₃N or TEA triethylamine
EtOH ethyl alcohol
FA formic acid
h or hr hour
HATU 1-[bis(dimethylamino)methylene]-1H-1,2,3-triazolo[4,5-b]pyridinium 3-oxid hexafluorophosphate
HOAt 1-hydroxy-7-azabenzotriazole
HOBt or HOBT 1-hydroxybenzotriazole hydrate
iPr Isopropyl
MeOH methyl alcohol
Me₄t-BuXphos ditert-butyl-[2,3,4,5-tetramethyl-6-(2,4,6-triisopropylphenyl)phenyl]phosphane
min minute
MTBE tert-butyl methyl ether
n-BuLi n-butylithium
NMP 1-methyl-2-pyrrolidinone
OAc acetate
Pd/C palladium on carbon
PDC pyridinium dichromate
PdCl₂(dtbpf) or Pd(dtbpf)Cl₂ dichloro[1,1′-bis(di-t-butylphosphino)ferrocene]palladium(II)
PdCl₂(dppf) or Pd(dppf)Cl₂[1,1′-bis(diphenylphosphino)ferrocene]dichloropalladium(II)
Pd₂(dba)₃ tris(dibenzylideneacetone)dipalladium(0)
Pd(PPh₃)₄ tetrakis(triphenylphosphine)palladium(0
Pd(PPh₃)₂Cl₂ dichlorobis(triphenylphosphine)palladium(II)
PE petroleum ether
PPh₃ triphenylphosphine
Pr n-propyl
Py pyridine
rac racemic
Rf retention factor
r.t. or rt room temperature
sat. saturated
SFC supercritical fluid chromatography
t-Bu tert-butyl
tBuXphos-Pd-G3 or [2-(2-aminophenyl)phenyl]-methylsulfonyloxypalladium; ditert-tBuXphos Pd G₃ or butyl-[2-(2,4,6-triisopropylphenyl)phenyl]phosphane t-BuXphos-Pd (gen 3)
TFA trifluoroacetic acid
Tf₂O trifluoromethanesulfonic anhydride
THF tetrahydrofuran
TLC thin layer chromatography
Xantphos-Pd-G3 [2-(2-aminophenyl)phenyl]-methylsulfonyloxy-palladium; (5-diphenylphosphanyl-9,9-dimethyl-xanthen-4-yl)-diphenyl-phosphane

### Example 1. Preparation of Intermediates

### Preparation of (2S,4R)-1-[(2S)-2-(10-aminodecanamido)-3,3-dimethylbutanoyl]-4-hydroxy-N-[[4-(4-methyl-1,3-thiazol-5-yl)phenyl]methyl]pyrrolidine-2-carboxamide (I-1)

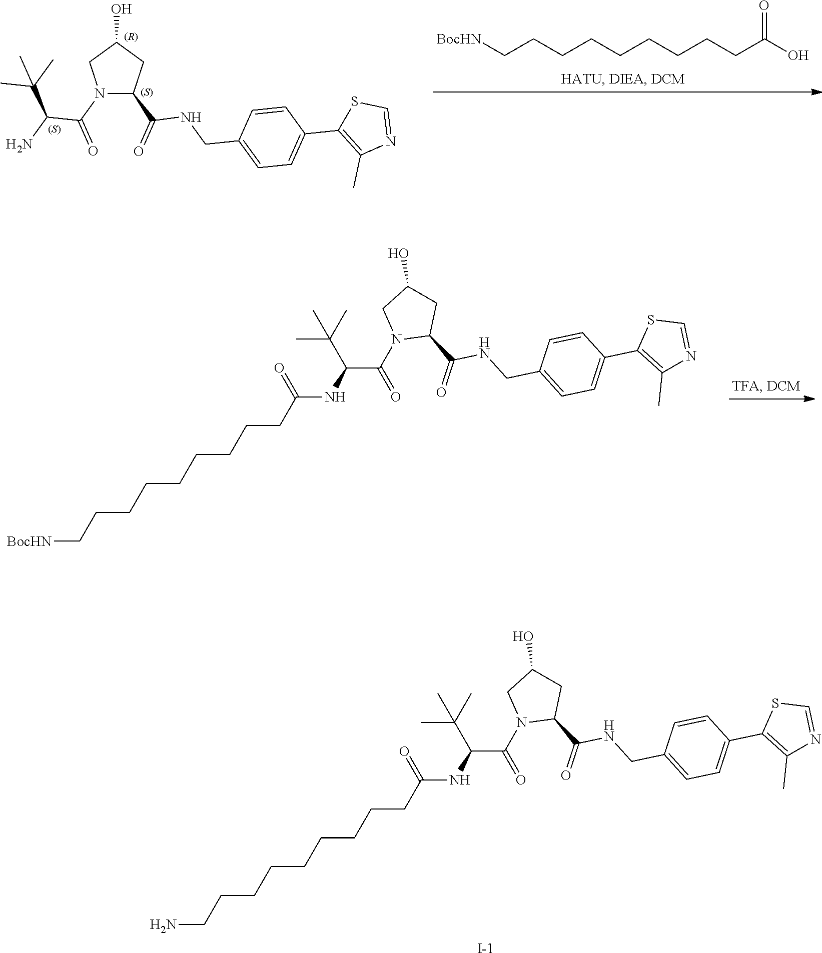

### Step 1: Preparation of tert-butyl N-(9-[[(2S)-1-[(2S,4R)-4-hydroxy-2-([[4-(4-methyl-1,3-thiazol-5-yl)phenyl]methyl]carbamoyl)pyrrolidin-1-yl]-3,3-dimethyl-1-oxobutan-2-yl]carbamoyl]nonyl)carbamate

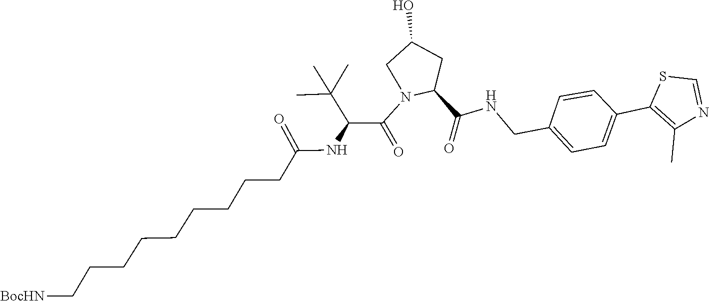 [SMILES: Cc1ncsc1-c1ccc(CNC(=O)[C@@H]2C[C@@H](O)CN2C(=O)[C@@H](NC(=O)CCCCCCCCCNC(=O)OC(C)(C)C)C(C)(C)C)cc1]

To a stirred mixture of (2S,4R)-1-[(2S)-2-amino-3,3-dimethylbutanoyl]-4-hydroxy-N-[[4-(4-methyl-1,3-thiazol-5-yl)phenyl]methyl]pyrrolidine-2-carboxamide (1.00 g, 0.002 mmol, 1.00 equiv) and 10-[(tert-butoxycarbonyl)amino]decanoic acid (0.73 g, 0.003 mmol, 1.10 equiv) in DCM (20.00 mL) was added DIEA (0.90 g, 0.007 mmol, 3.00 equiv). The mixture was stirred at room temperature for 5 min, then HATU (1.32 g, 0.003 mmol, 1.50 equiv) was added. After stirring at room temperature for 2 h, to the mixture was added water (100 mL) and the mixture was extracted with DCM (100 mL×4). The organic fractions were combined and dried over anhydrous sodium sulfate and filtered, and the filtrate was concentrated to give crude product that was purified by chromatography on silica gel, eluting with DCM/MeOH (from 100:0 DCM:MeOH ratio to 100:7 DCM:MeOH ratio) to give tert-butyl N-(9-[[(2S)-1-[(2S,4R)-4-hydroxy-2-([[4-(4-methyl-1,3-thiazol-5-yl)phenyl]methyl]carbamoyl)pyrrolidin-1-yl]-3,3-dimethyl-1-oxobutan-2-yl]carbamoyl]nonyl)carbamate (1.67 g, 92.46%). LCMS (ESI) m/z [M+H]⁺=700.

### Step 2: Preparation of (2S,4R)-1-[(2S)-2-(10-aminodecanamido)-3,3-dimethylbutanoyl]-4-hydroxy-N-[[4-(4-methyl-1,3-thiazol-5-yl)phenyl]methyl]pyrrolidine-2-carboxamide (I-1)

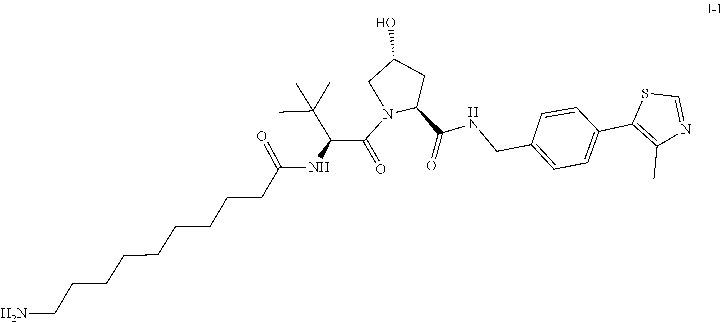 [SMILES: Cc1ncsc1-c1ccc(CNC(=O)[C@@H]2C[C@@H](O)CN2C(=O)[C@@H](NC(=O)CCCCCCCCCN)C(C)(C)C)cc1]

To a stirred solution of tert-butyl N-(9-[[(2S)-1-[(2S,4R)-4-hydroxy-2-([[4-(4-methyl-1,3-thiazol-5-yl)phenyl]methyl]carbamoyl)pyrrolidin-1-yl]-3,3-dimethyl-1-oxobutan-2-yl]carbamoyl]nonyl)carbamate (1.67 g, 2.386 mmol, 1 equiv) in DCM (10 mL) was added TFA (2 mL, 26.926 mmol, 11.29 equiv) and the mixture was stirred for 2 h at room temperature. The resulting mixture was concentrated under vacuum. A solution of was 5% K₂CO₃ (MeOH/water=5/2) added and the pH adjusted to 8-9. The mixture was stirred for 2 h at room temperature. The final mixture was concentrated under vacuum and the crude material was purified by reversed phase flash chromatography (C18 silica gel column), eluting with 10-35% acetonitrile in 0.1% aqueous NH₄HCO₃ over 20 min, detected at UV 254 nm, to provide (2S,4R)-1-[(2S)-2-(10-aminodecanamido)-3,3-dimethylbutanoyl]-4-hydroxy-N-[[4-(4-methy-1,3-thiazol-5-yl)phenyl]methyl]pyrrolidine-2-carboxamide (I-1, 1.11 g, 73.68%). LCMS (ESI) m/z: [M+H]⁺=600.40.

The following intermediates in Table A1 were prepared by a route analogous to that used for the preparation of intermediate I-1.

TABLE A1

| | | | LCMS |
|---|---|---|---|
| | Intermediate | | (ESI) m/z: |
| Structure | No. | Name | [M + H]⁺ |
| 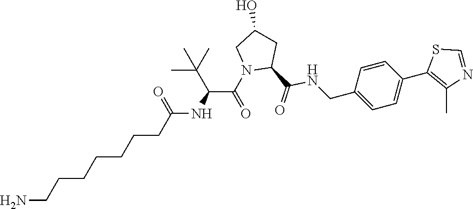 [SMILES: Cc1ncsc1-c1ccc(CNC(=O)[C@@H]2C[C@@H](O)CN2C(=O)[C@@H](NC(=O)CCCCCCCN)C(C)(C)C)cc1] | I-2 | (2S,4R)-1-[(2S)-2-(8- aminooctanamido)-3,3- dimethylbutanoyl]-4-hydroxy- N-[[4-(4-methyl-1,3-thiazol-5- yl)phenyl]methyl]pyrrolidine-2- carboxamide | 572.2 |
| 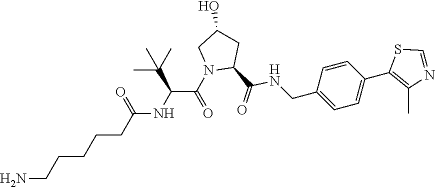 [SMILES: Cc1ncsc1-c1ccc(CNC(=O)[C@@H]2C[C@@H](O)CN2C(=O)[C@@H](NC(=O)CCCCCN)C(C)(C)C)cc1] | I-3 | (2S,4R)-1-[(2S)-2-(6- aminohexanamido)-3,3- dimethylbutanoyl]-4-hydroxy- N-[[4-(4-methyl-1,3-thiazol-5- yl)phenyl]methyl]pyrrolidine-2- carboxamide | 544.4 |
| 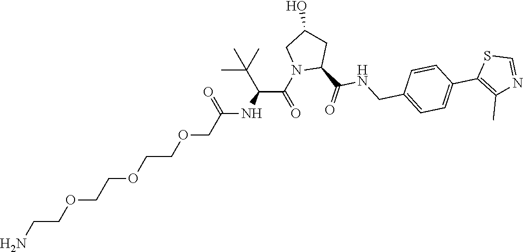 [SMILES: Cc1ncsc1-c1ccc(CNC(=O)[C@@H]2C[C@@H](O)CN2C(=O)[C@@H](NC(=O)COCCOCCOCCN)C(C)(C)C)cc1] | I-4 | (2S,4R)-1-((S)-14-amino-2- (tert-butyl)-4-oxo-6,9,12-trioxa- 3-azatetradecanoyl)-4- hydroxy-N-(4-(4-methylthiazol- 5-yl)benzyl)pyrrolidine-2- carboxamide | 620.3 |
| 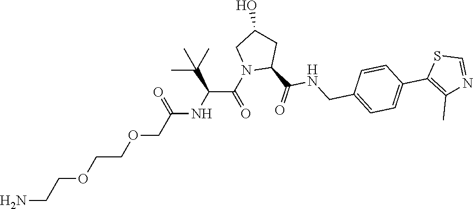 [SMILES: Cc1ncsc1-c1ccc(CNC(=O)[C@@H]2C[C@@H](O)CN2C(=O)[C@@H](NC(=O)COCCOCCN)C(C)(C)C)cc1] | I-5 | (2S,4R)-1-[(2S)-2-[2-[2-(2- aminoethoxy)ethoxy] acetamido]-3,3- dimethylbutanoyl]-4- hydroxy-N-[[4-(4-methyl-1,3- thiazol-5- yl)phenyl]methyl]pyrrolidine- 2-carboxamide | 576.3 |

### Preparation of 5-(2-(4-aminopiperidin-1-yl)ethoxy)-2-(2,6-dioxopiperidin-3-yl)isoindoline-1,3-dione FA (I-6)

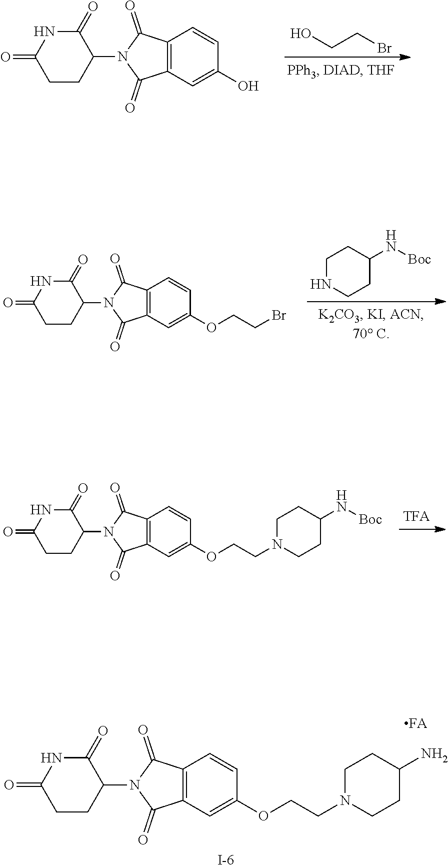

### Step 1: Preparation of 5-(2-bromoethoxy)-2-(2,6-dioxopiperidin-3-yl)isoindoline-1,3-dione

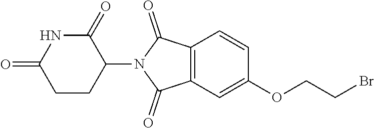 [SMILES: O=C1CCC(N2C(=O)c3ccc(OCCBr)cc3C2=O)C(=O)N1]

To a solution of 2-(2,6-dioxopiperidin-3-yl)-5-hydroxyisoindole-1,3-dione (1.37 g, 4.996 mmol, 1.00 equiv) in THF (35 mL) was added 2-bromoethanol (0.94 g, 7.494 mmol, 1.5 equiv), PPh₃ (1.97 g, 7.494 mmol, 1.5 equiv) and DIAD (1.52 g, 7.494 mmol, 1.5 equiv) at 0° C. The resulting mixture was stirred for 2 h at room temperature. The residue was purified by reversed phase flash chromatography (C18 silica gel column), eluting with 0-100% ACN in water over 45 min, to provide 5-(2-bromoethoxy)-2-(2,6-dioxopiperidin-3-yl)isoindoline-1,3-dione (1.52 g, 79.82%) as a black solid. LCMS (ESI) m/z: [M+H]⁺=381.38.

### Step 2: Preparation of tert-butyl (1-(2-((2-(2,6-dioxopiperidin-3-yl)-1,3-dioxoisoindolin-5-yl)oxy)ethyl)piperidin-4-yl)carbamate

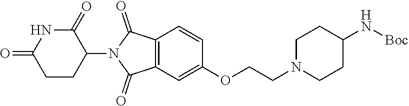 [SMILES: CC(C)(C)OC(=O)NC1CCN(CCOc2ccc3c(c2)C(=O)N(C2CCC(=O)NC2=O)C3=O)CC1]

To a solution of 5-(2-bromoethoxy)-2-(2,6-dioxopiperidin-3-yl)isoindoline-1,3-dione (1.52 g, 3.988 mmol, 1.00 equiv) in ACN (35.00 mL) was added tert-butyl N-(piperidin-4-yl)carbamate (0.80 g, 3.988 mmol, 1.00 equiv), KI (0.66 g, 3.988 mmol, 1.00 equiv) and K₂CO₃ (1.65 g, 11.963 mmol, 3.00 equiv). The resulting solution was stirred at 70° C. for 2 h. The residue was purified by reversed phase flash chromatography (C18 silica gel), eluting with 0-100% ACN in water over 30 min, to provide tert-butyl (1-(2-((2-(2,6-dioxopiperidin-3-yl)-1,3-dioxoisoindolin-5-yl)oxy)ethyl)piperidin-4-yl)carbamate (1.402 g, 70.24%) as a colorless solid. LCMS (ESI) m/z: [M+H]⁺=501.

### Step 3: Preparation of 5-(2-(4-aminopiperidin-1-yl)ethoxy)-2-(2,6-dioxopiperidin-3-yl)isoindoline-1,3-dione FA (I-6)

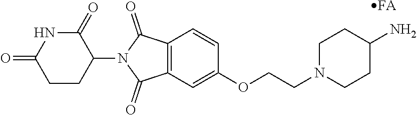

To a solution of tert-butyl (1-(2-((2-(2,6-dioxopiperidin-3-yl)-1,3-dioxoisoindolin-5-yl)oxy)ethyl)piperidin-4-yl)carbamate (1.66 g, 3.316 mmol, 1.00 equiv) in DCM (10.00 mL) was added TFA (10.00 mL, 134.630 mmol, 40.60 equiv). The resulting solution was stirred at room temperature for 3 h, then concentrated under reduced pressure. The residue was purified by reversed phase flash chromatography (C18 silica gel column), eluting with 0-100% ACN in 0.1% formic acid in water over 45 min, to give 5-(2-(4-aminopiperidin-1-yl)ethoxy)-2-(2,6-dioxopiperidin-3-yl)isoindoline-1,3-dione formate (I-6, 840 mg, 62.52%) as a white solid. LCMS (ESI) m/z: [M+H]⁺=401.17.

The following intermediates in Table A2 were prepared by a route analogous to that used for the preparation of intermediate I-6.

TABLE A2

| | | | LCMS |
|---|---|---|---|
| | Intermediate | | (ESI) m/z: |
| Structure | No. | Name | [M + H]⁺ |
| 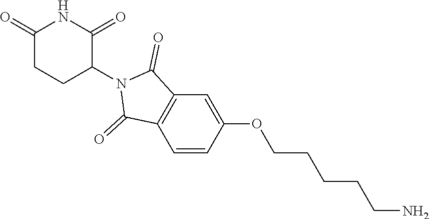 [SMILES: NCCCCCOc1ccc2c(c1)C(=O)N(C1CCC(=O)NC1=O)C2=O] | K-9 | 5-((5-aminopentyl)oxy)-2-(2,6- dioxopiperidin-3-yl)isoindoline- 1,3-dione | 360 |

### Preparation of 4-(azetidin-3-ylmethoxy)-2-(2,6-dioxopiperidin-3-yl)isoindoline-1,3-dione TFA (I-7)

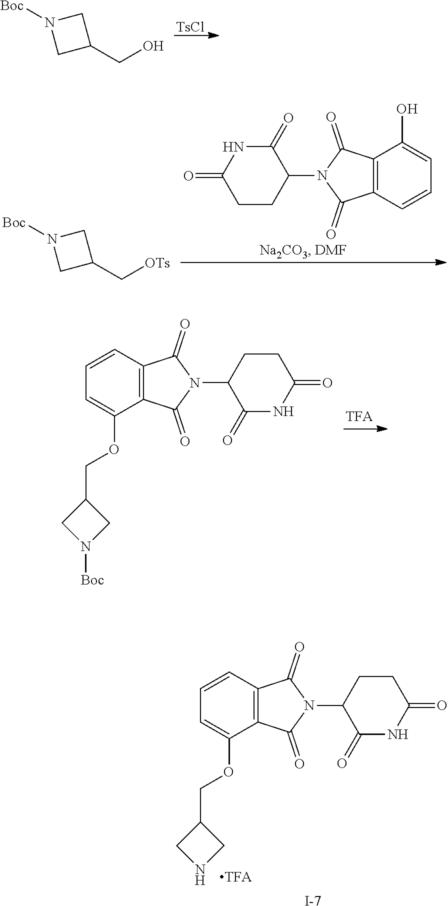

### Step 1: Preparation of tert-butyl 3-((tosyloxy)methyl)azetidine-1-carboxylate

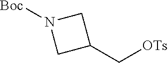 [SMILES: Cc1ccc(S(=O)(=O)OCC2CN(C(=O)OC(C)(C)C)C2)cc1]

To a stirred solution of tert-butyl 3-(hydroxymethyl)azetidine-1-carboxylate (1.87 g, 9.987 mmol, 1.00 equiv) in DCM (50.00 mL, 786.502 mmol, 78.75 equiv) was added DMAP (0.18 g, 1.498 mmol, 0.15 equiv), TEA (2.53 g, 24.968 mmol, 2.50 equiv) and p-toluenesulfonyl chloride (2.86 g, 14.981 mmol, 1.50 equiv) at 0° C. The resulting mixture was stirred for 2 h at 0° C., then allowed to warm to room temperature and stirred for an additional 5 h. The residue was purified by silica gel column chromatography, eluting with petroleum ether/THF (1:1), to afford tert-butyl 3-((tosyloxy)methyl)azetidine-1-carboxylate (2.65 g, 77.72%) as a colorless oil. LCMS (ESI) m/z: [M+H]⁺=342.

### Step 2: Preparation of tert-butyl 3-([[2-(2,6-dioxopiperidin-3-yl)-1,3-dioxoisoindol-4-yl]oxy]methyl)azetidine-1-carboxylate

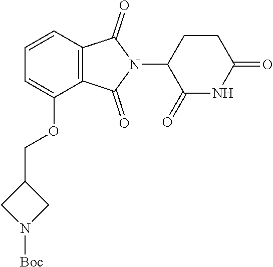 [SMILES: CC(C)(C)OC(=O)N1CC(COc2cccc3c2C(=O)N(C2CCC(=O)NC2=O)C3=O)C1]

To a solution of tert-butyl 3-((tosyloxy)methyl)azetidine-1-carboxylate (2.30 g, 8.387 mmol, 1.00 equiv) in DMF (15.00 mL) was added 2-(2,6-dioxopiperidin-3-yl)-4-hydroxyisoindoline-1,3-dione (2.86 g, 8.387 mmol, 1 equiv) and Na₂CO₃ (1.33 g, 12.581 mmol, 1.5 equiv). The resulting mixture was stirred at 80° C. for 5 h under an atmosphere of dry nitrogen. The reaction was quenched with water at room temperature. The resulting mixture was extracted with EtOAc (3×100 mL). The combined organic layers were washed with brine (3×100 mL) and dried over anhydrous Na₂SO₄. After filtration, the filtrate was concentrated under reduced pressure to provide tert-butyl 3-([[2-(2,6-dioxopiperidin-3-yl)-1,3-dioxoisoindol-4-yl]oxy]methyl)azetidine-1-carboxylate (3.37 g, 90.61%) as a light yellow solid. LCMS (ESI) m/z: [M+H]⁺=444.

### Step 3: Preparation of 4-(azetidin-3-ylmethoxy)-2-(2,6-dioxopiperidin-3-yl)isoindoline-1,3-dione TFA (I-7)

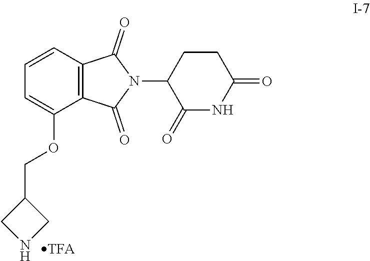 [SMILES: O=C1CCC(N2C(=O)c3cccc(OCC4CNC4)c3C2=O)C(=O)N1]

To a solution of tert-butyl 3-([[2-(2,6-dioxopiperidin-3-yl)-1,3-dioxoisoindol-4-yl]oxy]methyl)azetidine-1-carboxylate (2.39 g, 5.389 mmol, 1.00 equiv) in DCM (10.00 mL) was added TFA (10.00 mL, 134.630 mmol, 42.34 equiv). The resulting solution was stirred at room temperature for 3 h. The residue was purified by reversed phase flash chromatography (C18 silica gel column), eluting with 0-100% ACN in water over 45 min, to provide 4-(azetidin-3-ylmethoxy)-2-(2,6-dioxopiperidin-3-yl)isoindoline-1,3-dione TFA (I-7, 1.712 g, 87.72%) as a light yellow solid. LCMS (ESI) m/z: [M+H]⁺=344.12.

The following intermediates in Table A3 were prepared by a route analogous to that used for the preparation of intermediate I-7.

TABLE A3

| | | | LCMS |
|---|---|---|---|
| | Intermediate | | (ESI) m/z: |
| Structure | No. | Name | [M + H]⁺ |
| 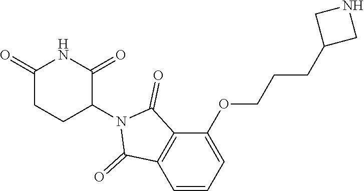 [SMILES: O=C1CCC(N2C(=O)c3cccc(OCCCC4CNC4)c3C2=O)C(=O)N1] | K-10 | 4-[3-(azetidin-3-yl)propoxy]-2- (2,6-dioxopiperidin-3- yl)isoindole-1,3-dione | 372 |

### Preparation of 5-(azetidin-3-ylmethoxy)-2-(2,6-dioxopiperidin-3-yl)isoindole-1,3-dione TFA (I-8)

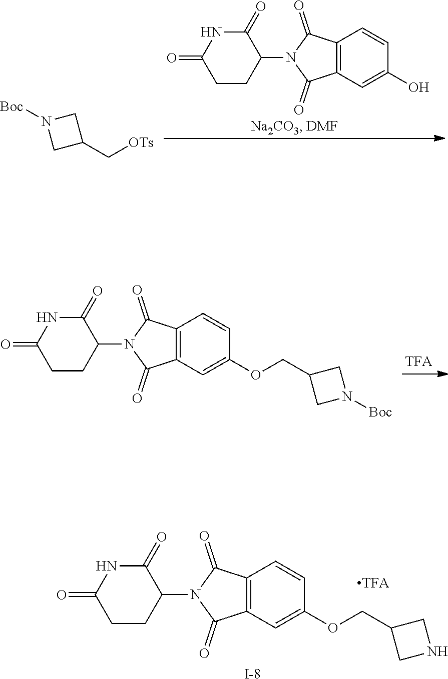

### Step 1: Preparation of tert-butyl 3-([[2-(2,6-dioxopiperidin-3-yl)-1,3-dioxoisoindol-5-yl]oxy]methyl)azetidine-1-carboxylate

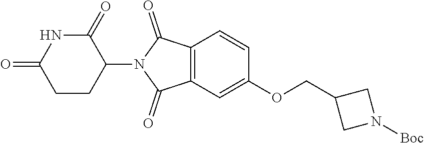 [SMILES: CC(C)(C)OC(=O)N1CC(COc2ccc3c(c2)C(=O)N(C2CCC(=O)NC2=O)C3=O)C1]

To a solution of tert-butyl 3-((tosyloxy)methyl)azetidine-1-carboxylate (2.50 g, 9.116 mmol, 1.00 equiv) in DMF (15.00 mL) was added 2-(2,6-dioxopiperidin-3-yl)-5-hydroxyisoindoline-1,3-dione (3.11 g, 9.116 mmol, 1 equiv) and Na₂CO₃ (1.45 g, 13.675 mmol, 1.5 equiv). The resulting solution was stirred at 80° C. for 5 h under an atmosphere of dry nitrogen. The reaction was allowed to cool to room temperature and quenched with water, then extracted with EtOAc (3×100 mL). The combined organic layers were washed with brine (3×100 mL) and dried over anhydrous Na₂SO₄. After filtration, the filtrate was concentrated under reduced pressure. This resulted in tert-butyl 3-([[2-(2,6-dioxopiperidin-3-yl)-1,3-dioxoisoindol-5-yl]oxy]methyl)azetidine-1-carboxylate (2.15 g, 53.18%) as a light yellow solid. LCMS (ESI) m/z: [M+H]⁺=444.

### Step 2: Preparation of 5-(azetidin-3-ylmethoxy)-2-(2,6-dioxopiperidin-3-yl)isoindole-1,3-dione TFA (I-8)

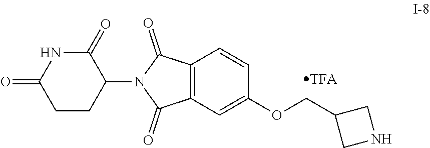 [SMILES: O=C1CCC(N2C(=O)c3ccc(OCC4CNC4)cc3C2=O)C(=O)N1]

To a solution of tert-butyl 3-([[2-(2,6-dioxopiperidin-3-yl)-1,3-dioxoisoindol-5-yl]oxy]methyl)azetidine-1-carboxylate (1.41 g, 3.180 mmol, 1.00 equiv) in DCM (10.00 mL) was added TFA (10.00 mL, 134.630 mmol, 42.34 equiv). The resulting solution was stirred at room temperature for 3 h, then concentrated under reduced pressure. The residue was purified by reversed phase flash chromatography (C18 silica gel column), eluting with 0-100% ACN in water over 45 min, to provide 5-(azetidin-3-ylmethoxy)-2-(2,6-dioxopiperidin-3-yl)isoindole-1,3-dione TFA (I-8, 906.2 mg, 81.38%) as a white solid. LCMS (ESI) m/z: [M+H]+=344.12.

### Preparation of 4-((3-((4-aminobutyl)sulfonyl)propyl)amino)-2-(2,6-dioxopiperidin-3-yl)isoindoline-1,3-dione FA (I-9)

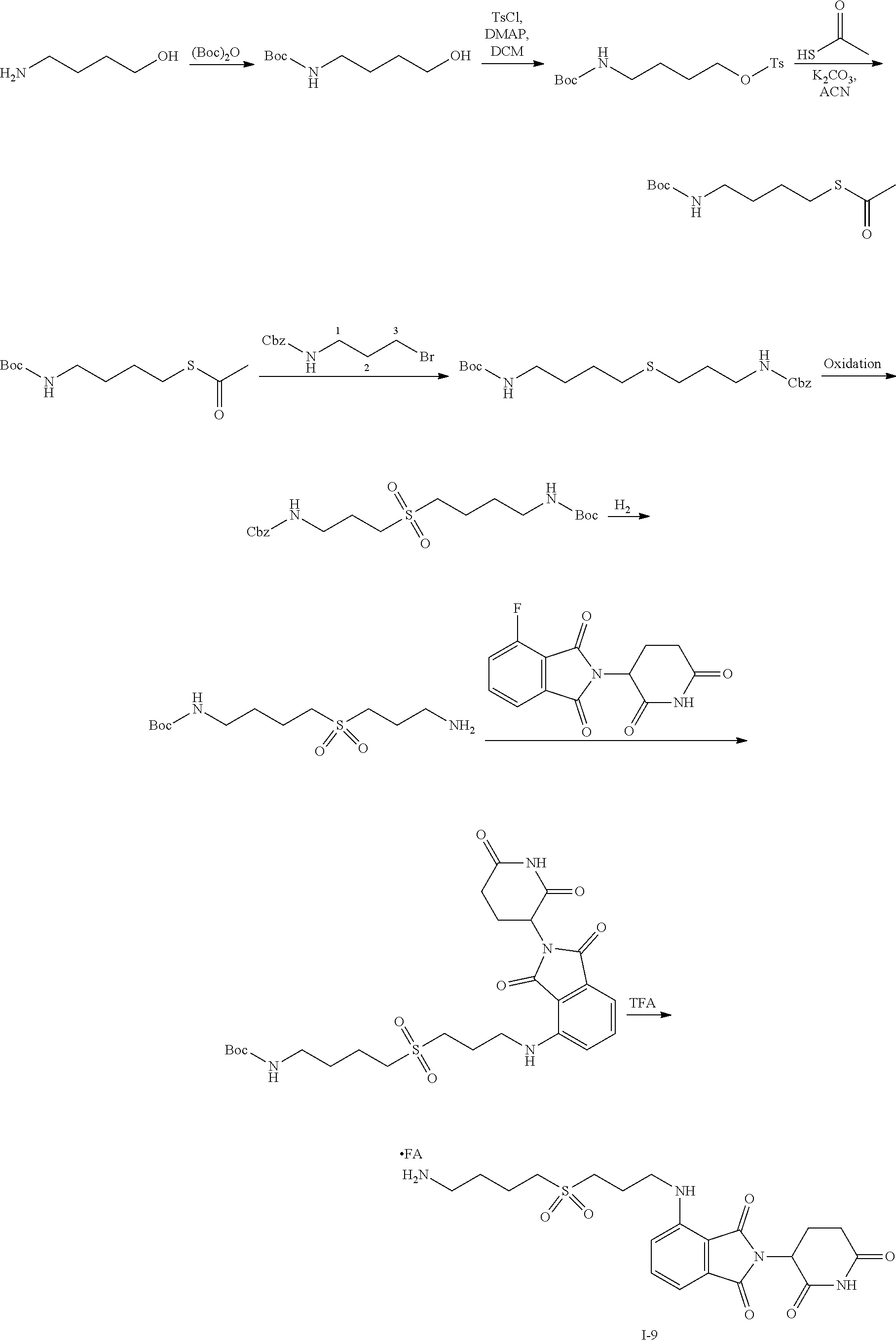

### Step 1: Preparation of tert-butyl N-(4-hydroxybutyl)carbamate

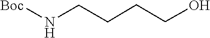 [SMILES: CC(C)(C)OC(=O)NCCCCO]

A solution of di-tert-butyl decarbonate (52.89 g, 242.321 mmol, 1.5 equiv) and 4-aminobutan-1-ol (14.40 g, 161.547 mmol, 1.00 equiv) in THF (160.00 mL) was stirred at room temperature for 1 h. The solution was concentrated to dryness, and the oily residue was purified by flash column chromatography (eluting with 40-60% EtOAc-hexane) to provide tert-butyl N-(4-hydroxybutyl)carbamate as a colorless oil that solidified to a white solid on standing. LCMS (ESI) m/z: [M+H]⁺=190.

### Step 2: Preparation of 4-((tert-butoxycarbonyl)amino)butyl 4-methylbenzenesulfonate

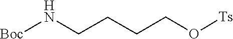 [SMILES: Cc1ccc(S(=O)(=O)OCCCCNC(=O)OC(C)(C)C)cc1]

To a stirred solution of tert-butyl N-(4-hydroxybutyl)carbamate (30.58 g, 161.581 mmol, 1.00 equiv) in DCM (400.00 mL) was added DMAP (2.96 g, 24.237 mmol, 0.15 equiv), TEA (40.88 g, 403.952 mmol, 2.5 equiv) and p-toluenesulfonyl chloride (46.21 g, 242.371 mmol, 1.5 equiv) at 0° C. The resulting mixture was stirred for 2 h at 0° C., then 5 h at room temperature. The solution was concentrated under reduced pressure and the residue was purified by silica gel column chromatography, eluting with petroleum ether/THF (1:1) to afford 4-((tert-butoxycarbonyl)amino)butyl 4-methylbenzenesulfonate (45.6 g, 82.17%) as a light-yellow oil. LCMS (ESI) m/z: [M+H]⁺=344.

### Step 3: Preparation of S-(4-((tert-butoxycarbonyl)amino)butyl) ethanethioate

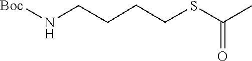 [SMILES: CC(=O)SCCCCNC(=O)OC(C)(C)C]

To a stirred solution of 4-((tert-butoxycarbonyl)amino)butyl 4-methylbenzenesulfonate (45.60 g, 132.774 mmol, 1.00 equiv) in ACN (300.00 mL) was added ethanethioic S-acid (15.16 g, 199.161 mmol, 1.5 equiv), and K₂CO₃ (55.05 g, 398.323 mmol, 3 equiv). The resulting mixture was stirred for 12 h at room temperature. The mixture was concentrated under reduced pressure and the residue was purified by silica gel column chromatography, eluting with petroleum ether/THF (1:1) to afford S-(4-((tert-butoxycarbonyl)amino)butyl) ethanethioate (28.7 g, 87.39%) as a light-yellow oil. LCMS (ESI) m/z: [M+H]⁺=248.

### Step 4: Preparation of benzyl N-[3-([4-[(tert-butoxycarbonyl)amino]butyl]sulfanyl)propyl]carbamate

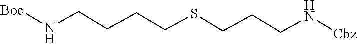 [SMILES: CC(C)(C)OC(=O)NCCCCSCCCNC(=O)OCc1ccccc1]

To a solution of S-(4-((tert-butoxycarbonyl)amino)butyl) ethanethioate (3.60 g, 14.554 mmol, 1.00 equiv) in MeOH (90.00 mL, 2222.902 mmol, 152.73 equiv) was added benzyl (3-bromopropyl)carbamate (4.36 g, 16.010 mmol, 1.1 equiv) and NaOMe (3.15 g, 58.217 mmol, 4 equiv). The resulting solution was stirred at room temperature for 3 h. The reaction was quenched with water at room temperature. The resulting mixture was extracted with EtOAc (3×100 mL). The combined organic layers were washed with brine (3×100 mL) and dried over anhydrous Na₂SO₄. After filtration, the filtrate was concentrated under reduced pressure. The residue was purified by reversed phase flash chromatography (C18 silica gel column), eluting with 0-100% ACN in NH₄HCO₃ in water, to provide benzyl N-[3-([4-[(tert-butoxycarbonyl)amino]butyl]sulfanyl)propyl]carbamate (4.122 g, 71.42%) as a light yellow oil. LCMS (ESI) m/z: [M+H]⁺=397.

### Step 5: Preparation of benzyl N-(3-[4-[(tert-butoxycarbonyl)amino]butanesulfonyl]propyl)carbamate

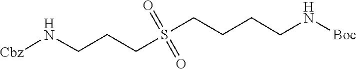 [SMILES: CC(C)(C)OC(=O)NCCCCS(=O)(=O)CCCNC(=O)OCc1ccccc1]

To a solution of benzyl N-[3-([4-[(tert-butoxycarbonyl)amino]butyl]sulfanyl)propyl]carbamate (4.13 g, 10.415 mmol, 1.00 equiv) in MeOH (60.00 mL) was added Oxone® (3.50 g, 20.840 mmol, 2 equiv). The resulting solution was stirred at room temperature for 12 h then concentrated under reduced pressure. The residue was purified by reversed phase flash chromatography (C18 silica gel column), eluting with 0-100% ACN in NH₄HCO₃ in water over 30 min, to provide benzyl N-(3-[4-[(tert-butoxycarbonyl)amino]butanesulfonyl]propyl)carbamate (2 g, 44.81%) as a white solid. LCMS (ESI) m/z: [M+H]⁺=429.

### Step 6: Preparation of tert-butyl (4-((3-aminopropyl)sulfonyl)butyl)carbamate

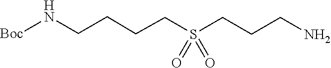 [SMILES: CC(C)(C)OC(=O)NCCCCS(=O)(=O)CCCN]

To a solution of benzyl N-(3-[4-[(tert-butoxycarbonyl)amino]butanesulfonyl]propyl)carbamate (1.95 g, 4.550 mmol, 1.00 equiv) in EtOH (30.00 mL) was added ammonium formate (573.85 mg, 9.101 mmol, 2 equiv) and 5% Pd(OH)₂/C (977.68 mg, 6.962 mmol, 1.53 equiv). The resulting solution was stirred at 60° C. for 12 h under one atmosphere of hydrogen. The resulting mixture was filtered, the filter cake was washed with MeOH (3×30 mL), and the filtrate was concentrated under reduced pressure to provide tert-butyl (4-((3-aminopropyl)sulfonyl)butyl)carbamate (1.12 g, crude) as a black solid. LCMS (ESI) m/z: [M+H]⁺=295.

### Step 7: Preparation of tert-butyl (4-((3-((2-(2,6-dioxopiperidin-3-yl)-1,3-dioxoisoindolin-4-yl)amino)propyl)sulfonyl)butyl)carbamate

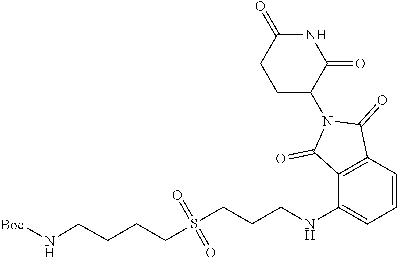 [SMILES: CC(C)(C)OC(=O)NCCCCS(=O)(=O)CCCNc1cccc2c1C(=O)N(C1CCC(=O)NC1=O)C2=O]

To a solution of tert-butyl (4-((3-aminopropyl)sulfonyl)butyl)carbamate (1.12 g, 3.804 mmol, 1.00 equiv) in NMP (30.00 mL) was added 2-(2,6-dioxopiperidin-3-yl)-4-fluoroisoindole-1,3-dione (1.05 g, 3.804 mmol, 1 equiv) and DIEA (1.48 g, 11.413 mmol, 3 equiv). The resulting solution was stirred at 90° C. for 3 h under an atmosphere of dry nitrogen. The resulting mixture was diluted with water (30 mL) and extracted with EtOAc (3×50 mL). The combined organic layers were washed with brine (3×30 mL) and dried over anhydrous Na₂SO₄. After filtration, the filtrate was concentrated under reduced pressure. The residue was purified by reversed phase flash chromatography (C18 silica gel column), eluting with 0-100% ACN in 0.1% formic acid in water over 45 min, to provide tert-butyl (4-((3-((2-(2,6-dioxopiperidin-3-yl)-1,3-dioxoisoindolin-4-yl)amino)propyl)sulfonyl)butyl)carbamate (970 mg, 46.31%) as a yellow solid. LCMS (ESI) m/z: [M+H]⁺=551.

### Step 8: Preparation of 4-((3-((4-aminobutyl)sulfonyl)propyl)amino)-2-(2,6-dioxopiperidin-3-yl)isoindoline-1,3-dione FA (I-9)

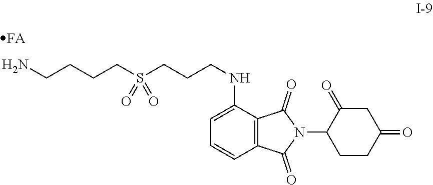 [SMILES: "·FA" ; NCCCCS(=O)(=O)CCCNc1cccc2c1C(=O)N(C1CCC(=O)CC1=O)C2=O]

To a solution of tert-butyl (4-((3-((2-(2,6-dioxopiperidin-3-yl)-1,3-dioxoisoindolin-4-yl)amino)propyl)sulfonyl)butyl)carbamate (970.00 mg, 1.762 mmol, 1.00 equiv) was added 4N dry HCl in dioxane (5.00 mL, 164.559 mmol, 93.41 equiv). The resulting solution was stirred at room temperature for 3 h and solvent was removed under reduced pressure. The residue was purified by reversed phase flash chromatography (C18 silica gel column), eluting with 0-100% ACN in 0.1% formic acid in water over 45 min, to provide 4-((3-((4-aminobutyl)sulfonyl)propyl)amino)-2-(2,6-dioxopiperidin-3-yl)isoindoline-1,3-dione FA (I-9, 712 mg, 89.17%) as a yellow solid. LCMS (ESI) m/z: [M+H]⁺=451.16.

### Preparation of 4-[[2-(2-aminoethanesulfonyl)ethyl]amino]-2-(2,6-dioxopiperidin-3-yl)isoindole-1,3-dione TFA (I-10)

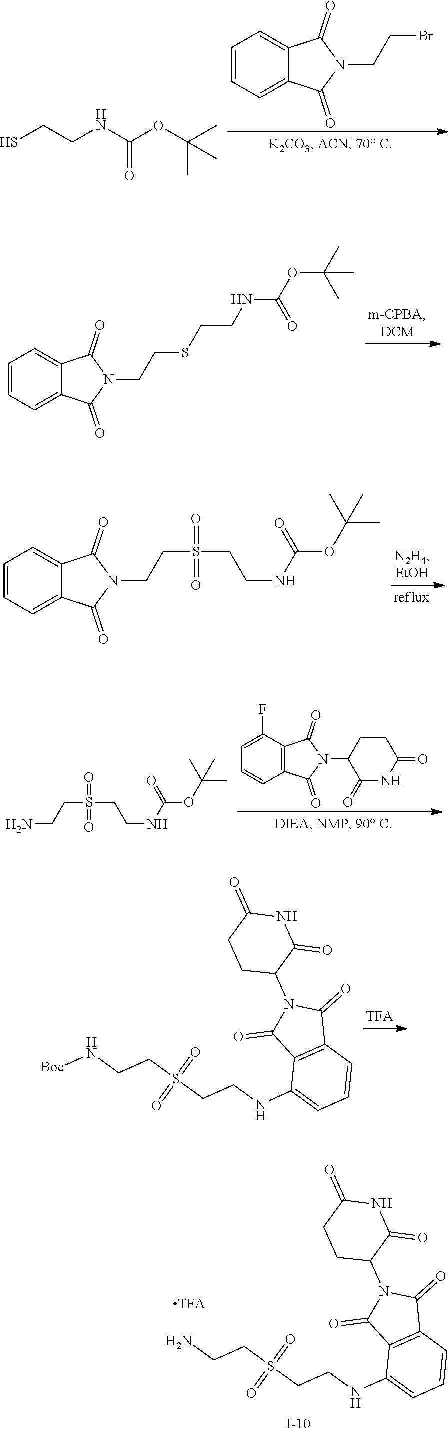

### Step 1: Preparation of tert-butyl N-(2-[[2-(1,3-dioxoisoindol-2-yl)ethyl]sulfanyl]ethyl)carbamate

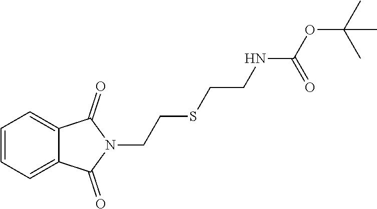 [SMILES: CC(C)(C)OC(=O)NCCSCCN1C(=O)c2ccccc2C1=O]

To a stirred mixture of tert-butyl N-(2-sulfanylethyl)carbamate (5.00 g, 28.207 mmol, 1.00 equiv) and N-(2-bromoethyl)phthalimide (7.17 g, 0.028 mmol, 1.00 equiv) in ACN (10.00 mL) was added K₂CO₃ (11.70 g, 0.085 mmol, 3.00 equiv) at 70° C. under an atmosphere of dry nitrogen. After 5 h, the resulting mixture was extracted with EtOAc (3×500 mL). The combined organic layers were washed with brine (3×300 mL) and dried over anhydrous Na₂SO₄. After filtration, the filtrate was concentrated under reduced pressure. The residue was purified by silica gel column chromatography, eluted with petroleum ether/EtOAc (5:1 to 1:1) to afford tert-butyl N-(2-[[2-(1,3-dioxoisoindol-2-yl)ethyl]sulfanyl]ethyl)carbamate (8.20 g, 82.96%) as a white solid. LCMS (ESI) m/z: [M+H]⁺=351.

### Step 2: Preparation of tert-butyl N-[2-[2-(1,3-dioxoisoindol-2-yl)ethanesulfonyl]ethyl]carbamate

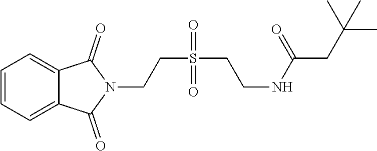 [SMILES: CC(C)(C)CC(=O)NCCS(=O)(=O)CCN1C(=O)c2ccccc2C1=O]

To a stirred mixture of tert-butyl N-(2-[[2-(1,3-dioxoisoindol-2-yl)ethyl]sulfanyl]ethyl)carbamate (8.20 g, 23.400 mmol, 1.00 equiv) in DCM (100 mL) was added m-CPBA (12.11 g, 70.199 mmol, 3.00 equiv) at room temperature under an atmosphere of dry nitrogen. Then reaction was quenched with saturated aqueous Na₂S₂O₃ (100 mL) at room temperature. To the resulting mixture was added saturated aqueous NaHCO₃ (100 mL) and the mixture was extracted with EtOAc (3×400 mL). The organic phase was separated and dried over anhydrous Na₂SO₄. After filtration, the filtrate was concentrated under reduced pressure. The residue was purified by silica gel column chromatography, eluting with petroleum ether/EtOAc (5:1 to 1:1) to afford tert-butyl N-[2-[2-(1,3-dioxoisoindol-2-yl)ethanesulfonyl]ethyl]carbamate (8.40 g, 87.30%) as a white solid. LCMS (ESI) m/z: [M+H]⁺=383.

### Step 3: Preparation of tert-butyl N-[2-(2-aminoethanesulfonyl)ethyl]carbamate

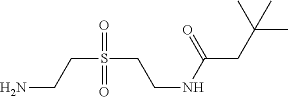 [SMILES: CC(C)(C)CC(=O)NCCS(=O)(=O)CCN]

To a stirred mixture of tert-butyl N-[2-[2-(1,3-dioxoisoindol-2-yl)ethanesulfonyl]ethyl]carbamate (3.40 g, 8.891 mmol, 1.00 equiv) in EtOH (100 mL) was added hydrazine hydrate (0.89 g, 17.781 mmol, 2.00 equiv) at 80° C. under an atmosphere of dry nitrogen. The resulting mixture was stirred for 1 h at 80° C. under an atmosphere of dry nitrogen. The resulting mixture was filtered, the filter cake was washed with EtOH (100 mL), and the filtrate was concentrated under reduced pressure to provide tert-butyl N-[2-(2-aminoethanesulfonyl)ethyl]carbamate (1.88 g, 77.94%) as a white solid. LCMS (ESI) m/z: [M+H]⁺=253.

### Step 4: Preparation of tert-butyl (2-((2-((2-(2,6-dioxopiperidin-3-yl)-1,3-dioxoisoindolin-4-yl)amino)ethyl)sulfonyl)ethyl)carbamate

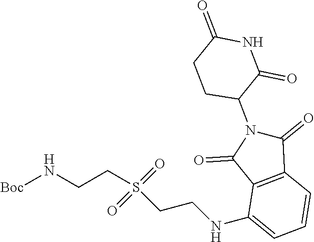 [SMILES: CC(C)(C)OC(=O)NCCS(=O)(=O)CCNc1cccc2c1C(=O)N(C1CCC(=O)NC1=O)C2=O]

To a stirred mixture of tert-butyl N-[2-(2-aminoethanesulfonyl)ethyl]carbamate (1.88 g, 7.451 mmol, 1.00 equiv) and 2-(2,6-dioxopiperidin-3-yl)-4-fluoroisoindole-1,3-dione (2.26 g, 8.196 mmol, 1.10 equiv) in NMP (25.00 mL) was added DIEA (2.89 g, 22.352 mmol, 3.00 equiv) dropwise at 90° C. under an atmosphere of dry nitrogen and the solution was stirred for 12 hr. The resulting mixture was cooled and extracted with EtOAc (3×300 mL). The combined organic layers were washed with saturated brine (3×100 mL) and dried over anhydrous Na₂SO₄. After filtration, the filtrate was concentrated under reduced pressure. The residue was purified by silica gel column chromatography, eluting with petroleum ether/EtOAc (1:1) to afford tert-butyl (2-((2-((2-(2,6-dioxopiperidin-3-yl)-1,3-dioxoisoindolin-4-yl)amino)ethyl)sulfonyl)ethyl)carbamate (1.58 g, 40.03%) as a yellow solid. LCMS (ESI) m/z: [M+H]⁺=509.

### Step 5: Preparation of 4-[[2-(2-aminoethanesulfonyl)ethyl]amino]-2-(2,6-dioxopiperidin-3-yl)isoindole-1,3-dione TFA (I-10)

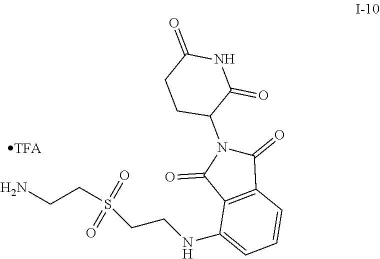 [SMILES: NCCS(=O)(=O)CCNc1cccc2c1C(=O)N(C1CCC(=O)NC1=O)C2=O]

To a stirred mixture of tert-butyl (2-((2-((2-(2,6-dioxopiperidin-3-yl)-1,3-dioxoisoindolin-4-yl)amino)ethyl)sulfonyl)ethyl)carbamate (1.54 g, 3.028 mmol, 1.00 equiv) in DCM (20 mL) was added TFA (5.0 mL) dropwise at 25° C. under an atmosphere of dry nitrogen. After 1 hr, the resulting mixture was concentrated under vacuum to give intermediate I-10 (1.68 g, 122.25%) as a yellow solid. LCMS (ESI) m/z: [M+H]⁺=409.11.

### Preparation of 3-[5-([2-[2-(2-aminoethoxy)ethoxy]ethyl]amino)-2-methyl-4-oxoquinazolin-3-yl]piperidine-2,6-dione TFA (I-11)

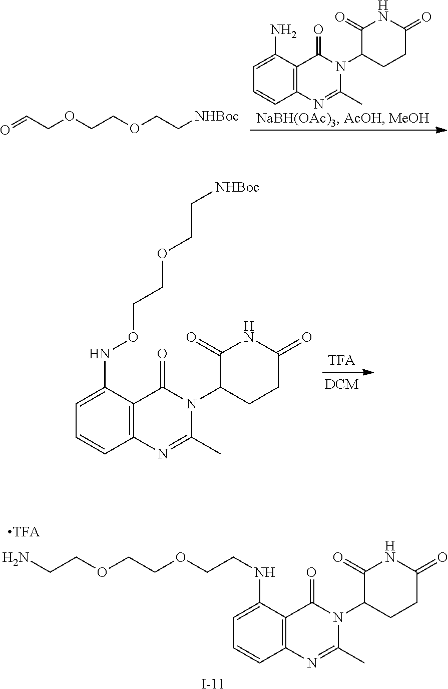

### Step 1: Preparation of tert-butyl (2-(2-(2-((3-(2,6-dioxopiperidin-3-yl)-2-methyl-4-oxo-3,4-dihydroquinazolin-5-yl)amino)ethoxy)ethoxy)ethyl)carbamate

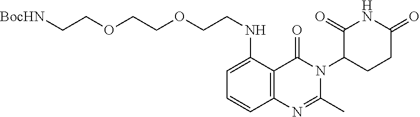 [SMILES: Cc1nc2cccc(NCCOCCOCCNC(=O)OC(C)(C)C)c2c(=O)n1C1CCC(=O)NC1=O]

To a stirred solution of 4-hydroxy-2,2-dimethyl-3,8,11-trioxa-5-azatridecan-13-al (1.25 g, 4.995 mmol, 1 equiv) and 3-(5-amino-2-methyl-4-oxoquinazolin-3-yl)piperidine-2,6-dione (1.43 g, 4.995 mmol, 1.00 equiv) in MeOH (30.00 mL) was added NaBH₃CN (0.63 g, 9.990 mmol, 2 equiv) at room temperature and the resulting mixture was stirred for 1 h. The reaction was quenched with saturated aqueous NH₄Cl at 0° C., the solvent was evaporated, and the resulting residue was purified by silica gel column chromatography, eluting with petroleum ether/EtOAc (9:1) to afford tert-butyl (2-(2-(2-((3-(2,6-dioxopiperidin-3-yl)-2-methyl-4-oxo-3,4-dihydroquinazolin-5-yl)amino)ethoxy)ethoxy)ethyl)carbamate (1.29 g, 49.70%) as a yellow solid. LCMS (ESI) m/z [M+H]⁺=518.

### Step 2: Preparation of 3-[5-([2-[2-(2-aminoethoxy)ethoxy]ethyl]amino)-2-methyl-4-oxoquinazolin-3-yl]piperidine-2,6-dione TFA (I-11)

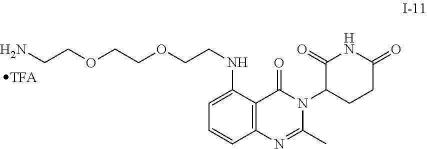 [SMILES: Cc1nc2cccc(NCCOCCOCCN)c2c(=O)n1C1CCC(=O)NC1=O]

A solution of tert-butyl (2-(2-(2-((3-(2,6-dioxopiperidin-3-yl)-2-methyl-4-oxo-3,4-dihydroquinazolin-5-yl)amino)ethoxy)ethoxy)ethyl)carbamate (1.29 g, 2.483 mmol, 1.00 equiv) and TFA (8.49 g, 74.481 mmol, 30 equiv) in DCM (6.00 mL) was stirred for 1 h at room temperature. The reaction mixture was concentrated, and the resulting residue was purified by reversed phase flash chromatography with the following conditions (column: C18 silica gel; mobile phase: ACN in water, 10% to 50% gradient in 10 min; detector, UV 254 nm) to afford 3-[5-([2-[2-(2-aminoethoxy)ethoxy]ethyl]amino)-2-methyl-4-oxoquinazolin-3-yl]piperidine-2,6-dione TFA (I-11, 1.56 g, 95.13%) as a light brown solid. LCMS (ESI) m/z: [M+H]⁺=418.20.

### Preparation of 3-[4-([2-[2-(2-aminoethoxy)ethoxy]ethyl]amino)-1-oxo-3H-isoindol-2-yl]piperidine-2,6-dione TFA (I-12)

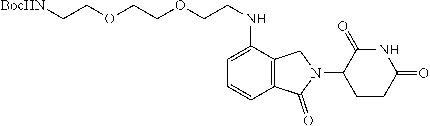 [SMILES: CC(C)(C)OC(=O)NCCOCCOCCNc1cccc2c1CN(C1CCC(=O)NC1=O)C2=O]

### Step 1: Preparation of tert-butyl (2-(2-(2-((2-(2,6-dioxopiperidin-3-yl)-1-oxoisoindolin-4-yl)amino)ethoxy)ethoxy)ethyl)carbamate

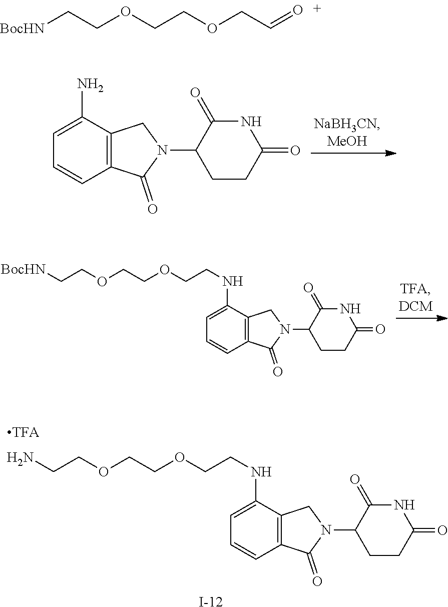

To a stirred solution of tert-butyl (2-(2-(2-oxoethoxy)ethoxy)ethyl)carbamate (1.33 g, 0.005 mmol, 1.00 equiv) and lenalidomide (1.38 g, 5.323 mmol, 1.00 equiv) in MeOH (20.00 mL) was added NaBH₃CN (0.67 g, 0.011 mmol, 2.00 equiv) at room temperature and the resulting mixture was stirred for 1 h. Then, the reaction was quenched with saturated aqueous NH₄Cl at 0° C., the solvent was evaporated, and the resulting residue was purified by silica gel column chromatography, eluting with petroleum ether/EtOAc (9:1) to afford tert-butyl (2-(2-(2-((2-(2,6-dioxopiperidin-3-yl)-1-oxoisoindolin-4-yl)amino)ethoxy)ethoxy)ethyl)carbamate (0.92 g, 34.12%) as a yellow solid. LCMS (ESI) m/z [M+H]⁺=491.

### Step 2: Preparation of 3-[4-([2-[2-(2-aminoethoxy)ethoxy]ethyl]amino)-1-oxo-3H-isoindol-2-yl]piperidine-2,6-dione TFA (I-12)

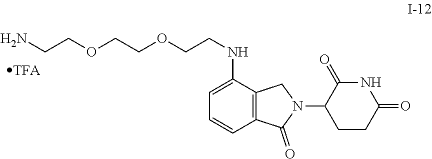 [SMILES: NCCOCCOCCNc1cccc2c1CN(C1CCC(=O)NC1=O)C2=O]

A solution of tert-butyl (2-(2-(2-((2-(2,6-dioxopiperidin-3-yl)-1-oxoisoindolin-4-yl)amino)ethoxy)ethoxy)ethyl)carbamate (1.20 g, 2.436 mmol, 1.00 equiv) and TFA (5.56 g, 48.724 mmol, 20.00 equiv) in DCM (5.00 mL) was stirred for 1 h at room temperature. The reaction mixture was concentrated, and the resulting residue was purified by reversed phase flash chromatography (C18 silica gel column), eluting with 10-50% ACN in water over 10 min, detected at UV 254 nm, to afford 3-[4-([2-[2-(2-aminoethoxy)ethoxy]ethyl]amino)-1-oxo-3H-isoindol-2-yl]piperidine-2,6-dione TFA (I-12, 979 mg, 93.87%) as a yellow solid. LCMS (ESI) m/z: [M+H]⁺=391.19.

### Preparation of 3-(5-((6-aminohexyl)amino)-2-methyl-4-oxoquinazolin-3(4H)-yl)piperidine-2,6-dione trifluoroacetate (I-13)

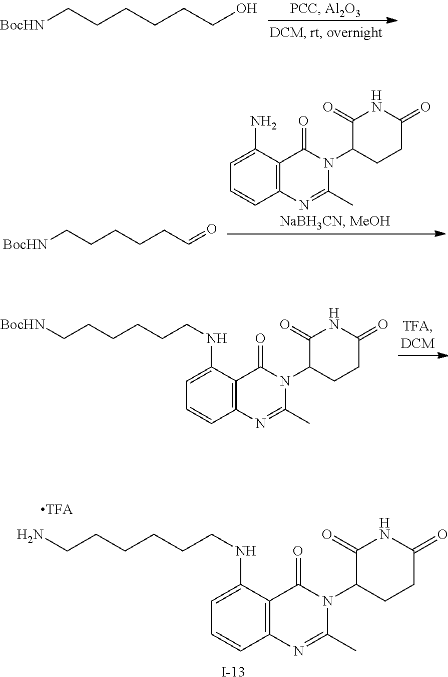

### Step 1: Preparation of tert-butyl N-(6-oxohexyl)carbamate

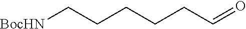 [SMILES: CC(C)(C)OC(=O)NCCCCCC=O]

To a stirred solution of tert-butyl (6-hydroxyhexyl)carbamate (800.00 mg, 3.681 mmol, 1.00 equiv) in DCM (10.00 mL) was added PCC (1190.31 mg, 5.522 mmol, 1.50 equiv) and aluminum oxide (75.07 mg, 0.736 mmol, 0.20 equiv). The resulting mixture was stirred overnight at room temperature. The solution was concentrated and the residue was purified by silica gel column chromatography, eluting with DCM/MeOH (10:1) to afford tert-butyl N-(6-oxohexyl)carbamate (500 mg, 63.09%) as a yellow oil.

### Step 2: Preparation of tert-butyl (6-((3-(2,6-dioxopiperidin-3-yl)-2-methyl-4-oxo-3,4-dihydroquinazolin-5-yl)amino)hexyl)carbamate

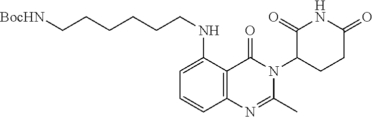 [SMILES: Cc1nc2cccc(NCCCCCCNC(=O)OC(C)(C)C)c2c(=O)n1C1CCC(=O)NC1=O]

To a stirred solution of tert-butyl N-(6-oxohexyl)carbamate (380.00 mg, 1.765 mmol, 1.00 equiv) and 3-(5-amino-2-methyl-4-oxoquinazolin-3(4H)-yl)piperidine-2,6-dione (252.66 mg, 0.883 mmol, 0.50 equiv) in MeOH (5.00 mL) was added NaBH₃CN (221.84 mg, 3.530 mmol, 2.00 equiv). The resulting mixture was stirred for 2 h at room temperature. The solution was concentrated and purified by Preparative TLC (eluting with DCM/MeOH, 20:1) to afford tert-butyl (6-((3-(2,6-dioxopiperidin-3-yl)-2-methyl-4-oxo-3,4-dihydroquinazolin-5-yl)amino)hexyl)carbamate (200 mg, 23.34%) as a yellow solid.

LCMS (ESI) m/z: [M+H]⁺=486.

### Step 3: Preparation of 3-(5-((6-aminohexyl)amino)-2-methyl-4-oxoquinazolin-3(4H)-yl)piperidine-2,6-dione TFA (I-13)

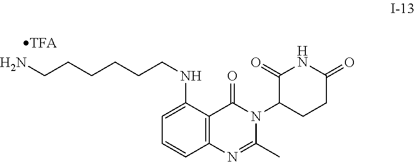 [SMILES: Cc1nc2cccc(NCCCCCCN)c2c(=O)n1C1CCC(=O)NC1=O]

To a stirred solution of tert-butyl (6-((3-(2,6-dioxopiperidin-3-yl)-2-methyl-4-oxo-3,4-dihydroquinazolin-5-yl)amino)hexyl)carbamate (100 mg) in DCM (1.00 mL) was added TFA (0.50 mL). The resulting mixture was stirred for 1 h at room temperature. The resulting mixture was concentrated under reduced pressure to provide 3-(5-((6-aminohexyl)amino)-2-methyl-4-oxoquinazolin-3(4H)-yl)piperidine-2,6-dione trifluoroacetate (80 mg, 83.98%) as a yellow solid. LCMS (ESI) m/z: [M+H]⁺=386.21.

### Preparation of 3-(5-((8-aminooctyl)amino)-2-methyl-4-oxoquinazolin-3(4H)-yl)piperidine-2,6-dione TFA (I-14)

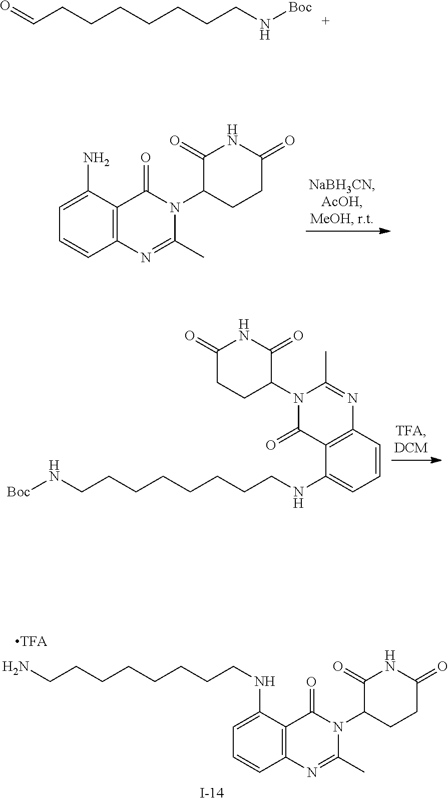

### Step 1: Preparation of tert-butyl (8-((3-(2,6-dioxopiperidin-3-yl)-2-methyl-4-oxo-3,4-dihydroquinazolin-5-yl)amino)octyl)carbamate

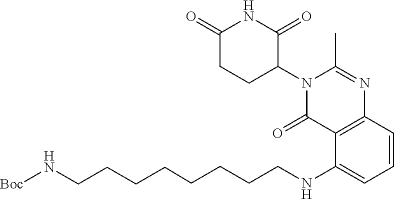 [SMILES: Cc1nc2cccc(NCCCCCCCCNC(=O)OC(C)(C)C)c2c(=O)n1C1CCC(=O)NC1=O]

To a stirred mixture of 3-(5-amino-2-methyl-4-oxoquinazolin-3(4H)-yl)piperidine-2,6-dione (500.00 mg, 1.746 mmol, 1.00 equiv) and tert-butyl (8-oxooctyl)carbamate (850.00 mg, 3.493 mmol, 2.00 equiv) in MeOH was added AcOH (350.00 mg, 5.828 mmol, 3.34 equiv) at room temperature. The resulting mixture was stirred for 2 h at room temperature and NaBH₃CN (9.22 mg, 0.147 mmol, 2.00 equiv) was then added at room temperature. The resulting mixture was stirred for an additional 1 h at room temperature. The residue was purified by reversed phase flash chromatography (C18 silica gel column), eluting with 0-100% ACN in water over 30 min, to provide tert-butyl (8-((3-(2,6-dioxopiperidin-3-yl)-2-methyl-4-oxo-3,4-dihydroquinazolin-5-yl)amino)octyl)carbamate (420 mg, 46.82%) as yellow solid. LCMS (ESI) m/z: [M+H]⁺=514.

### Step 2: Preparation of 3-(5-((8-aminooctyl)amino)-2-methyl-4-oxoquinazolin-3(4H)-yl)piperidine-2,6-dione TFA (I-14)

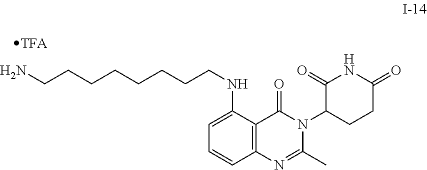 [SMILES: Cc1nc2cccc(NCCCCCCCCN)c2c(=O)n1C1CCC(=O)NC1=O]

To a solution of tert-butyl (8-((3-(2,6-dioxopiperidin-3-yl)-2-methyl-4-oxo-3,4-dihydroquinazolin-5-yl)amino)octyl)carbamate (1.54 g, 2.998 mmol, 1.00 equiv) in DCM (10.00 mL) was added TFA (10.00 mL, 134.630 mmol, 44.90 equiv). The resulting solution was stirred at room temperature for 6 h. The solution was concentrated and the residue was purified by reversed phase flash chromatography (C18 silica gel column), eluting with 0-100% ACN in water over 35 min, to provide 3-(5-((8-aminooctyl)amino)-2-methyl-4-oxoquinazolin-3(4H)-yl)piperidine-2,6-dione TFA (I-14, 1.4 g, 112.92%) as a light yellow solid. LCMS (ESI) m/z: [M+H]⁺=414.24.

### Preparation of 3-(4-((8-aminooctyl)amino)-1-oxoisoindolin-2-yl)piperidine-2,6-dione (I-15)

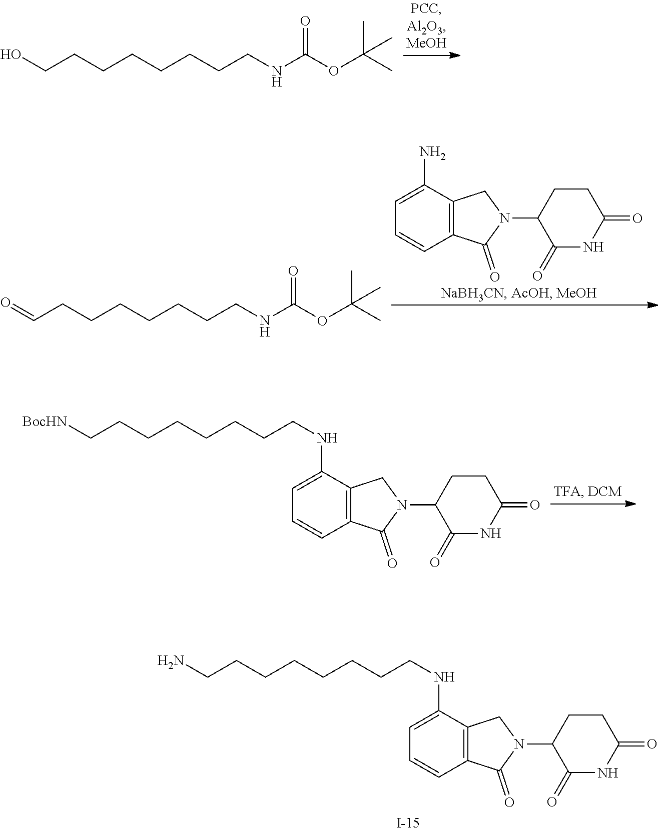

### Step 1: Preparation of tert-butyl (8-oxooctyl)carbamate

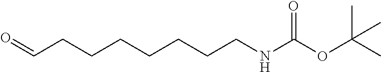 [SMILES: CC(C)(C)OC(=O)NCCCCCCCC=O]

To a stirred mixture of tert-butyl (8-hydroxyoctyl)carbamate (1.00 g, 4.076 mmol, 1.00 equiv) and PCC (878.51 mg, 4.076 mmol, 1.00 equiv) in MeOH (15.0 mL) was added Al₂O₃ (2.49 g, 24.454 mmol, 6.00 equiv) in portions at room temperature and the suspension was stirred for 2 h under an atmosphere of dry nitrogen. The precipitated solid was collected by filtration and washed with DCM (10×10 mL). The filtrate was concentrated under vacuum and the mixture was used in the next step directly without further purification. LCMS (ESI) m/z: [M+H]⁺=243.34.

### Step 2: Preparation of tert-butyl (8-((2-(2,6-dioxopiperidin-3-yl)-1-oxoisoindolin-4-yl)amino)octyl)carbamate

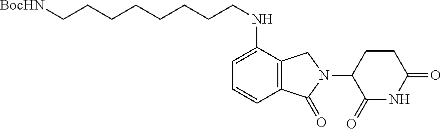 [SMILES: CC(C)(C)OC(=O)NCCCCCCCCNc1cccc2c1CN(C1CCC(=O)NC1=O)C2=O]

To a stirred mixture of tert-butyl (8-oxooctyl)carbamate (403.00 mg, 1.656 mmol, 1.00 equiv) and 3-(4-amino-1-oxoisoindolin-2-yl)piperidine-2,6-dione (429.36 mg, 1.656 mmol, 1.00 equiv) in MeOH (15.0 mL) was added NaBH₃CN (312.21 mg, 4.968 mmol, 3.00 equiv) and AcOH (0.01 mL, 0.158 mmol, 0.10 equiv) in portions at room temperature. The resulting mixture was stirred for 12 h under an atmosphere of dry nitrogen, then filtered. The filter cake was washed with MeOH (10×10 mL). The filtrate was concentrated under reduced pressure. The resulting mixture was purified by Preparative TLC (DCM/MeOH 40:1) to afford tert-butyl (8-((2-(2,6-dioxopiperidin-3-yl)-1-oxoisoindolin-4-yl)amino)octyl)carbamate (153 mg, 18.61%) as a dark green oil. LCMS (ESI) m/z: [M+H]⁺=486.61.

### Step 3: Preparation of 3-(4-((8-aminooctyl)amino)-1-oxoisoindolin-2-yl)piperidine-2,6-dione (I-15)

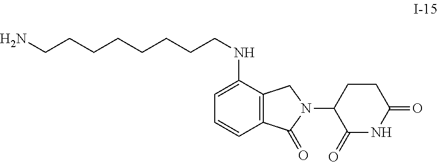 [SMILES: NCCCCCCCCNc1cccc2c1CN(C1CCC(=O)NC1=O)C2=O]

To a stirred mixture of tert-butyl (8-((2-(2,6-dioxopiperidin-3-yl)-1-oxoisoindolin-4-yl)amino)octyl)carbamate (153.00 mg, 0.314 mmol, 1.00 equiv) in DCM (4.00 mL) was added TFA (1.00 mL) at room temperature and the solution was stirred for 2 h under an atmosphere of dry nitrogen. The reaction mixture was concentrated under reduced pressure. The residue was purified by Preparative TLC (DCM/MeOH 40:1) to afford 3-(4-((8-aminooctyl)amino)-1-oxoisoindolin-2-yl)piperidine-2,6-dione (I-15, 110 mg, 88.71%) as a yellow solid. LCMS (ESI) m/z: [M+H]⁺=386.5.

### Preparation of 4-((8-aminooctyl)amino)-2-(2,6-dioxopiperidin-3-yl)isoindoline-1,3-dione TFA (I-16)

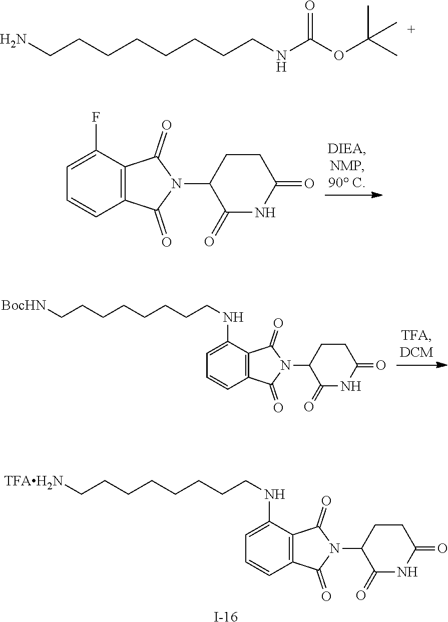

### Step 1: Preparation of tert-butyl (8-((2-(2,6-dioxopiperidin-3-yl)-1,3-dioxoisoindolin-4-yl)amino)octyl)carbamate

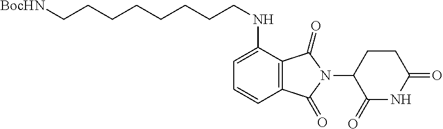 [SMILES: CC(C)(C)OC(=O)NCCCCCCCCNc1cccc2c1C(=O)N(C1CCC(=O)NC1=O)C2=O]

To a stirred solution of 2-(2,6-dioxopiperidin-3-yl)-4-fluoroisoindoline-1,3-dione (1.00 g, 3.620 mmol, 1.00 equiv) and tert-butyl (8-aminooctyl)carbamate (973.19 mg, 3.982 mmol, 1.10 equiv) in NMP (16 mL) was added DIEA (935.79 mg, 7.241 mmol, 2.00 equiv) at room temperature. The resulting mixture was stirred overnight at 90° C. The reaction was cooled to room temperature and quenched with water (10 mL). The aqueous layer was extracted with EtOAc (3×50 mL) and the combined organic phase was dried over sodium sulfate, filtered, and filtrate concentrated. The residue was purified by silica gel column chromatography, eluting with petroleum ether/EtOAc (1:1) to afford tert-butyl (8-((2-(2,6-dioxopiperidin-3-yl)-1,3-dioxoisoindolin-4-yl)amino)octyl)carbamate (1.19 g, 65.66%) as a light yellow solid. LCMS (ESI) m/z: [M+H]⁺=501.

### Step 2: Preparation of 4-((8-aminooctyl)amino)-2-(2,6-dioxopiperidin-3-yl)isoindoline-1,3-dione TFA (I-16)

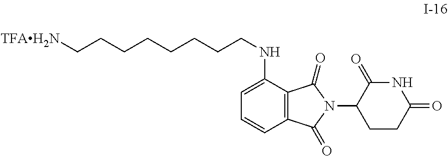

To a stirred solution of tert-butyl (8-((2-(2,6-dioxopiperidin-3-yl)-1,3-dioxoisoindolin-4-yl)amino)octyl)carbamate (300 mg, 0.599 mmol, 1 equiv), in DCM (10.00 mL) was added TFA (2.00 mL) dropwise at room temperature. The resulting mixture was stirred for 4 h at room temperature, then concentrated under reduced pressure. The residue was purified by CombiFlash to afford 4-((8-aminooctyl)amino)-2-(2,6-dioxopiperidin-3-yl)isoindoline-1,3-dione TFA (I-16, 130 mg, 54.17%) as a yellow solid. LCMS (ESI) m/z: [M+H]⁺=401.15.

### Preparation of 4-(2-([4,4′-bipiperidin]-1-yl)-2-oxoethoxy)-2-(2,6-dioxopiperidin-3-yl)isoindoline-1,3-dione (I-17)

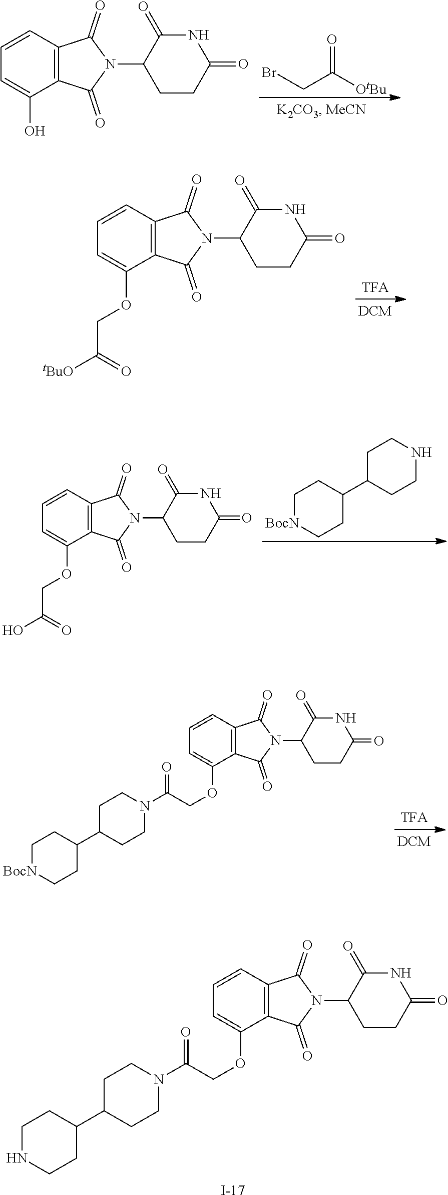

### Step 1: Preparation of tert-butyl 2-((2-(2,6-dioxopiperidin-3-yl)-1,3-dioxoisoindolin-4-yl)oxy)acetate

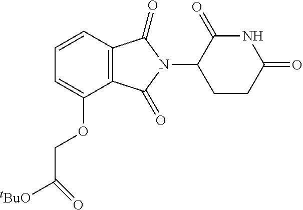 [SMILES: CC(C)(C)OC(=O)COc1cccc2c1C(=O)N(C1CCC(=O)NC1=O)C2=O]

To a stirred mixture of 2-(2,6-dioxopiperidin-3-yl)-4-hydroxyisoindoline-1,3-dione (10 g, 36.5 mmol, 1.0 equiv) and K₂CO₃ (7.56 g, 54.7 mmol, 1.5 equiv) in ACN (300 mL) was added tert-butyl-2-bromoacetate (7.82 g, 40.1 mmol, 1.1 equiv) at room temperature. The resulting mixture was stirred for 2 h at room temperature and then concentrated under reduced pressure. The residue was purified by reversed phase flash chromatography to afford tert-butyl 2-((2-(2,6-dioxopiperidin-3-yl)-1,3-dioxoisoindolin-4-yl)oxy)acetate (5.32 g, 37.56%) as a white solid. LCMS (ESI) m/z [M+H]⁺=389.

### Step 2: Preparation of 2-((2-(2,6-dioxopiperidin-3-yl)-1,3-dioxoisoindolin-4-yl)oxy)acetic acid

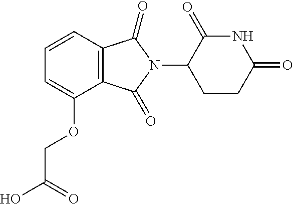 [SMILES: O=C(O)COc1cccc2c1C(=O)N(C1CCC(=O)NC1=O)C2=O]

To a stirred mixture of tert-butyl 2-((2-(2,6-dioxopiperidin-3-yl)-1,3-dioxoisoindolin-4-yl)oxy)acetate (5.32 g, 13.69 mmol, 1.00 equiv) in DCM (20 mL) was added TFA (4.0 mL) dropwise at room temperature. The resulting mixture was stirred for 2 h at room temperature. The crude solution was concentrated under reduced pressure. The residue was purified by reversed phase flash chromatography to provide 2-((2-(2,6-dioxopiperidin-3-yl)-1,3-dioxoisoindolin-4-yl)oxy)acetic acid (4.66 g, quant) as a white solid. LCMS (ESI) m/z [M+H]⁺=333.

### Step 3: Preparation of tert-butyl 1′-(2-((2-(2,6-dioxopiperidin-3-yl)-1,3-dioxoisoindolin-4-yl)oxy)acetyl)-[4,4′-bipiperidine]-1-carboxylate

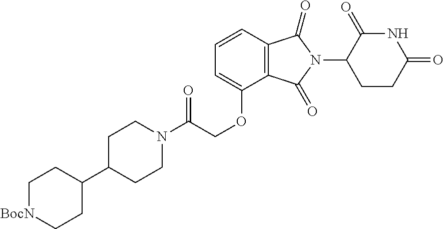 [SMILES: CC(C)(C)OC(=O)N1CCC(C2CCN(C(=O)COc3cccc4c3C(=O)N(C3CCC(=O)NC3=O)C4=O)CC2)CC1]

To a stirred mixture of 2-((2-(2,6-dioxopiperidin-3-yl)-1,3-dioxoisoindolin-4-yl)oxy)acetic acid (1.10 g, 3.311 mmol, 1.00 equiv) in DMF (10 mL) was added DIEA (8.54 g, 6.622 mmol, 2.00 equiv) and HATU (1.637 g, 4.30 mmol, 1.30 equiv) at room temperature under an atmosphere of dry nitrogen. The resulting mixture was stirred for 30 min and then tert-butyl[4,4-bipiperidine]-1-carboxylate (0.89 g, 3.311 mmol, 1.0 equiv) was added at room temperature. The resulting mixture was stirred for an additional 1 h then quenched with water (20 mL). The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by reversed phase flash chromatography to afford tert-butyl 1′-(2-((2-(2,6-dioxopiperidin-3-yl)-1,3-dioxoisoindolin-4-yl)oxy)acetyl)-[4,4′-bipiperidine]-1-carboxylate (1.36 g, 70.51%) as a yellow solid. LCMS (ESI) m/z [M+H]⁺=583.

### Step 4: Preparation of 4-(2-([4,4′-bipiperidin]-1-yl)-2-oxoethoxy)-2-(2,6-dioxopiperidin-3-yl)isoindoline-1,3-dione (I-17)

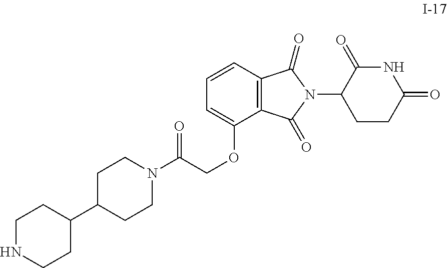 [SMILES: O=C1CCC(N2C(=O)c3cccc(OCC(=O)N4CCC(C5CCNCC5)CC4)c3C2=O)C(=O)N1]

To a stirred mixture of tert-butyl 1′-(2-((2-(2,6-dioxopiperidin-3-yl)-1,3-dioxoisoindolin-4-yl)oxy)acetyl)-[4,4′-bipiperidine]-1-carboxylate (1.36 g, 2.334 mmol, 1.0 equiv) in DCM (40 mL) was added TFA (10 mL) dropwise at room temperature. The resulting mixture was stirred for 2 h and then concentrated under reduced pressure. The residue was purified by reversed phase flash chromatography to yield 4-(2-([4,4′-bipiperidin]-1-yl)-2-oxoethoxy)-2-(2,6-dioxopiperidin-3-yl)isoindoline-1,3-dione (I-17, 780 mg, 69.25%) as a yellow solid. LCMS (ESI) m/z [M+H]⁺=483.

The following intermediates in Table A4 were prepared in a similar manner as described in the preparation of I-17.

TABLE A4

| | | | LCMS |
|---|---|---|---|
| | Intermediate | | (ESI)m/z: |
| Structure | No. | Name | [M + H]⁺ |
| 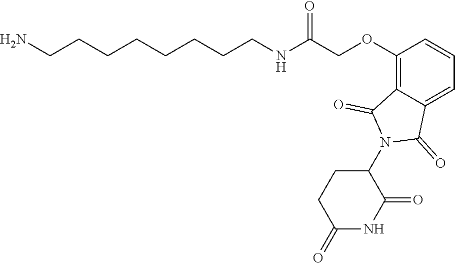 [SMILES: NCCCCCCCCNC(=O)COc1cccc2c1C(=O)N(C1CCC(=O)NC1=O)C2=O] | I-18 | N-(8-aminooctyl)-2-((2-(2,6- dioxopiperidin-3-yl)-1,3- dioxoisoindolin-4- yl)oxy)acetamide | 459.4 |

### Preparation of (2S,4R)-1-((S)-2-(4-(2-(2-aminoethoxy)ethoxy)butanamido)-3,3-dimethylbutanoyl)-4-hydroxy-N-(4-(4-methylthiazol-5-yl)benzyl)pyrrolidine-2-carboxamide (I-19)

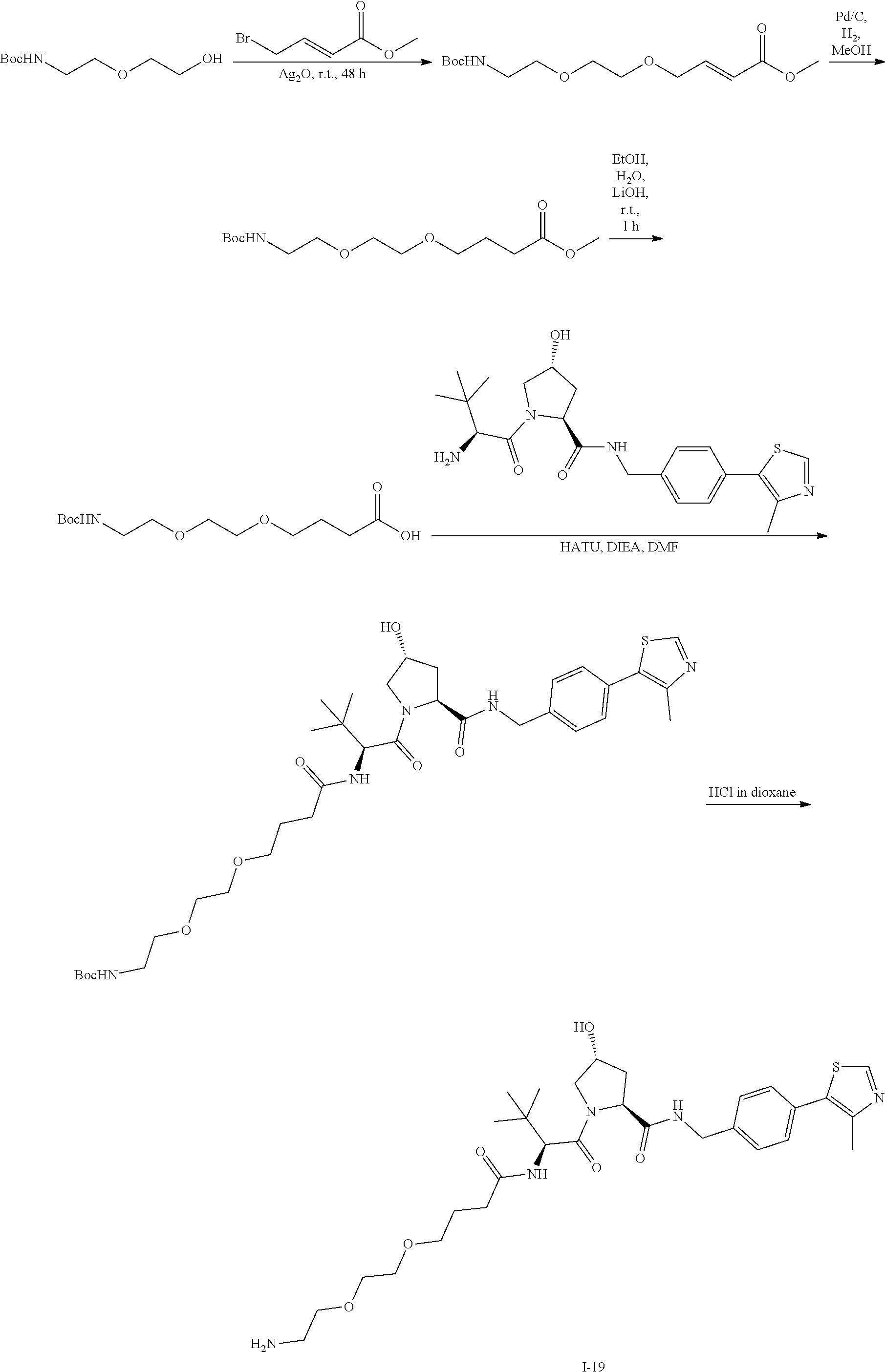

### Step 1: Preparation of methyl (2E)-4-(2-[2-[(tert-butoxycarbonyl)amino]ethoxy]ethoxy)but-2-enoate

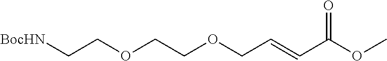 [SMILES: COC(=O)/C=C/COCCOCCNC(=O)OC(C)(C)C]

To a 40 mL sealed tube was added tert-butyl N-[2-(2-hydroxyethoxy)ethyl]carbamate (1.00 g, 4.872 mmol, 1.00 equiv), methyl (2E)-4-bromobut-2-enoate (8.77 g, 48.991 mmol, 10.06 equiv) and Ag₂O (3.22 g, 13.895 mmol, 2.85 equiv) at room temperature. The resulting mixture was stirred for 2 days at room temperature under an atmosphere of dry nitrogen. The resulting mixture was filtered, and the filter cake was washed with EtOAc (2×10 mL). The filtrate was concentrated under reduced pressure. The residue was purified by silica gel column chromatography, eluting with petroleum ether/EtOAc (1:1) to afford methyl (2E)-4-(2-[2-[(tert-butoxycarbonyl)amino]ethoxy]ethoxy)but-2-enoate (380 mg, 25.71%) as a yellow oil. LCMS (ESI) m/z: [M+H]⁺=304.

### Step 2: Preparation of methyl 2,2-dimethyl-4-oxo-3,8,11-trioxa-5-azapentadecan-15-oate

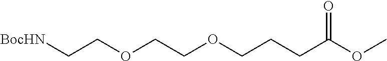 [SMILES: COC(=O)CCCOCCOCCNC(=O)OC(C)(C)C]

To a solution of methyl (2E)-4-(2-[2-[(tert-butoxycarbonyl)amino]ethoxy]ethoxy)but-2-enoate (380.00 mg, 1.253 mmol, 1.00 equiv) in MeOH (10.00 mL) was added 10% Pd/C (66.65 mg) under a nitrogen atmosphere in a 50 mL, 3-necked round-bottomed flask. The mixture was stirred for 4 h under one atmosphere of hydrogen. The reaction mixture was filtered through a Celite® pad and concentrated under reduced pressure to provide methyl 2,2-dimethyl-4-oxo-3,8,11-trioxa-5-azapentadecan-15-oate (300 mg, 78.43%) as a yellow oil that was used for next step without further purification.

### Step 3: Preparation of 2,2-dimethyl-4-oxo-3,8,11-trioxa-5-azapentadecan-15-oic acid

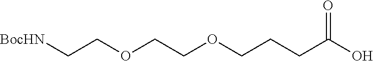 [SMILES: CC(C)(C)OC(=O)NCCOCCOCCCC(=O)O]

To a stirred solution of provide methyl 2,2-dimethyl-4-oxo-3,8,11-trioxa-5-azapentadecan-15-oate (280.00 mg, 0.917 mmol, 1.00 equiv) in MeOH (2.00 mL) was added a solution of LiOH (87.83 mg, 3.668 mmol, 4.00 equiv) in water (2.00 mL) dropwise at room temperature. The resulting mixture was stirred overnight at room temperature. The aqueous layer was extracted with EtOAc (2×10 mL). The aqueous layer was then acidified to pH 6 with HCl. The resulting mixture was extracted with EtOAc (2×10 mL). These combined organic layers were washed with water (30 mL) and dried over anhydrous Na₂SO₄. Upon filtration, the filtrate was concentrated under reduced pressure to provide 2,2-dimethyl-4-oxo-3,8,11-trioxa-5-azapentadecan-15-oic acid (100 mg) as a colorless oil that was directly used for next step. ¹H NMR (300 MHz, DMSO-d₆) δ 12.06 (s, 1H), 6.78 (d, J=8.7 Hz, 1H), 3.54-3.34 (m, 8H), 3.06 (qd, J=6.0, 3.3 Hz, 2H), 2.25 (t, J=7.4 Hz, 2H), 1.79-1.63 (m, 2H), 1.38 (d, J=1.3 Hz, 9H).

### Step 4: Preparation of tert-butyl N-[2-[2-(3-[[(2S)-1-[(2S,4R)-4-hydroxy-2-([[4-(4-methyl-1,3-thiazol-5-yl)phenyl]methyl]carbamoyl)pyrrolidin-1-yl]-3,3-dimethyl-1-oxobutan-2-yl]carbamoyl]propoxy)ethoxy]ethyl]carbamate

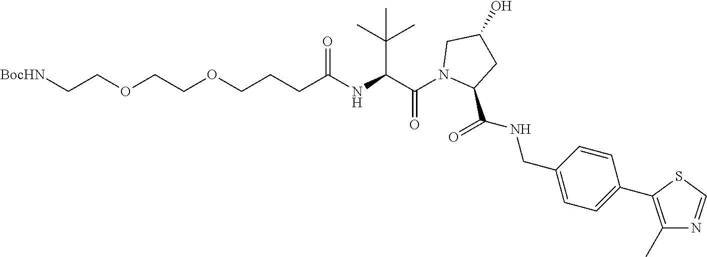 [SMILES: Cc1ncsc1-c1ccc(CNC(=O)[C@@H]2C[C@@H](O)CN2C(=O)[C@@H](NC(=O)CCCOCCOCCNC(=O)OC(C)(C)C)C(C)(C)C)cc1]

To a stirred mixture of 2,2-dimethyl-4-oxo-3,8,11-trioxa-5-azapentadecan-15-oic acid (400.00 mg, 1.373 mmol, 1.00 equiv) and HATU (626.44 mg, 1.648 mmol, 1.20 equiv) in DMF (10.00 mL) was added DIEA (212.93 mg, 1.648 mmol, 1.20 equiv) dropwise at room temperature under an atmosphere of dry nitrogen. The resulting mixture was stirred for 30 min. To the above mixture was added (2S,4R)-1-[(2S)-2-amino-3,3-dimethylbutanoyl]-4-hydroxy-N-[[4-(4-methyl-1,3-thiazol-5-yl)phenyl]methyl]pyrrolidine-2-carboxamide (472.92 mg, 1.098 mmol, 0.8 equiv) at room temperature. The resulting mixture was stirred overnight at room temperature, then partitioned between EtOAc (20 mL) and water (50 mL). The organic phase was separated and aqueous layer was extracted with EtOAc (2×10 mL). The combined organic layers were washed with brine (50 mL) and dried over anhydrous Na₂SO₄. After filtration, the filtrate was concentrated under reduced pressure. The residue was purified by silica gel column chromatography, eluting with DCM/MeOH (20:1) to afford tert-butyl N-[2-[2-(3-[[(2S)-1-[(2S,4R)-4-hydroxy-2-([[4-(4-methyl-1,3-thiazol-5-yl)phenyl]methyl]carbamoyl)pyrrolidin-1-yl]-3,3-dimethyl-1-oxobutan-2-yl]carbamoyl]propoxy)ethoxy]ethyl]carbamate (300 mg, 31.04%) as a yellow oil. LCMS (ESI) m/z: [M+H]⁺=704.

### Step 5: Preparation of (2S,4R)-1-((S)-2-(4-(2-(2-aminoethoxy)ethoxy)butanamido)-3,3-dimethylbutanoyl)-4-hydroxy-N-(4-(4-methylthiazol-5-yl)benzyl)pyrrolidine-2-carboxamide (I-19)

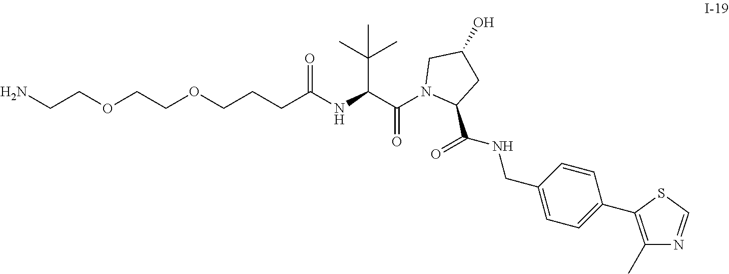 [SMILES: Cc1ncsc1-c1ccc(CNC(=O)[C@@H]2C[C@@H](O)CN2C(=O)[C@@H](NC(=O)CCCOCCOCCN)C(C)(C)C)cc1]

Into a 8 mL vial was added tert-butyl N-[2-[2-(3-[[(2S)-1-[(2S,4R)-4-hydroxy-2-([[4-(4-methyl-1,3-thiazol-5-yl)phenyl]methyl]carbamoyl)pyrrolidin-1-yl]-3,3-dimethyl-1-oxobutan-2-yl]carbamoyl]propoxy)ethoxy]ethyl]carbamate (300.00 mg, 0.426 mmol, 1.00 equiv) and 4 M HCl in dioxane (3.00 mL) at room temperature and the resulting mixture was stirred for 2 h. The solution was concentrated under reduced pressure to afford (2S,4R)-1-((S)-2-(4-(2-(2-aminoethoxy)ethoxy)butanamido)-3,3-dimethylbutanoyl)-4-hydroxy-N-(4-(4-methylthiazol-5-yl)benzyl)pyrrolidine-2-carboxamide (I-19, 300 mg) as a yellow oil that was used without further purification. LCMS (ESI) m/z: [M+H]⁺=604.

### Preparation of 4-(aminomethyl)-N-(3-chloro-1H-indol-7-yl)benzenesulfonamide (I-20) and 2-amino-N-([4-[(3-chloro-1H-indol-7-yl)sulfamoyl]phenyl]methyl)acetamide (I-21)

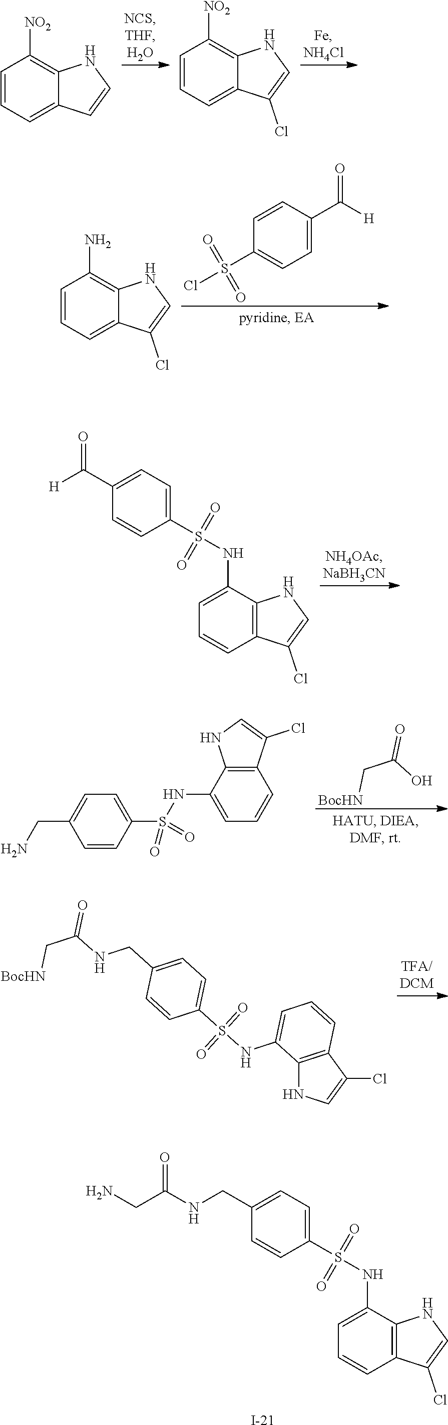

### Step 1: Preparation of 3-chloro-7-nitro-1H-indole

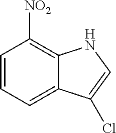 [SMILES: O=[N+]([O-])c1cccc2c(Cl)c[nH]c12]

To a mixture of 7-nitroindole (20.00 g, 123.344 mmol, 1.00 equiv) in THF (100.00 mL) and 0.1 N HCl (1.6 mL) was added NCS (16.47 g, 123.344 mmol, 1 equiv) in one portion. The reaction was stirred at room temperature for 5 h. Water (30 mL) was added. The resulting precipitate were collected by filtration; washed successively with water, MeOH/water (1:1), and isopropyl ether, and dried to afford 3-chloro-7-nitro-1H-indole (24.4 g). LCMS (ESI) m/z: [M+H]⁺=198.

### Step 2: Preparation of 3-chloro-1H-indol-7-amine

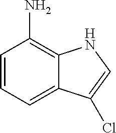 [SMILES: Nc1cccc2c(Cl)c[nH]c12]

To a suspension of 3-chloro-7-nitro-1H-indole (24.40 g, 124.116 mmol, 1.00 equiv) and iron powder (27.73 g, 496.465 mmol, 4 equiv) in IPA (350.00 mL) was added NH₄Cl (53.11 g, 992.929 mmol, 8 equiv) and water (350.00 mL). The resulting mixture was stirred for 2 h at 60° C. The resulting mixture was filtered and the filter cake was washed with EtOH (3×500 mL). The filtrate was concentrated under reduced pressure. The resulting mixture was diluted with EtOAc (500 mL) then was basified to pH 8 with saturated aqueousNaHCO₃. The resulting mixture was extracted with EtOAc (3×30 mL). The combined organic layers were washed with brine (3×500 mL) and dried over anhydrous Na₂SO₄. After filtration, the filtrate was concentrated under reduced pressure to afford 3-chloro-1H-indol-7-amine (16 g, 77.37%) as a black solid. LCMS (ESI) m/z: [M+H]⁺=167.

### Step 3: Preparation of N-(3-chloro-1H-indol-7-yl)-4-formylbenzenesulfonamide

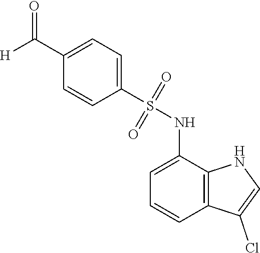 [SMILES: O=Cc1ccc(S(=O)(=O)Nc2cccc3c(Cl)c[nH]c23)cc1]

To 3-chloro-1H-indol-7-amine (1.55 g, 9.303 mmol, 1.00 equiv) in EtOAc (20.00 mL), 4-formylbenzenesulfonyl chloride (2.09 g, 10.233 mmol, 1.1 equiv) and pyridine (1.47 g, 18.606 mmol, 2 equiv) were added. The reaction mixture was stirred at room temperature for 3 h, diluted with EtOAc and washed successively with 1 N HCl, water, saturated aqueous solution of NaHCO₃, and brine. The organic layer was dried over MgSO₄, filtered and concentrated under vacuum. The crude was purified by flash column chromatography, using a gradient from 10% to 60% of EtOAc in heptane, to obtain N-(3-chloro-1H-indol-7-yl)-4-formylbenzenesulfonamide (1.90 g, 61.01%). LCMS (ESI) m/z: [M+H]⁺=335.

### Step 4: Preparation of 4-(aminomethyl)-N-(3-chloro-1H-indol-7-yl)benzenesulfonamide (I-20)

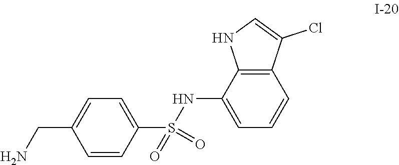 [SMILES: NCc1ccc(S(=O)(=O)Nc2cccc3c(Cl)c[nH]c23)cc1]

N-(3-chloro-1H-indol-7-yl)-4-formylbenzenesulfonamide (2.29 g, 6.841 mmol, 1.00 equiv) was dissolved in a saturated solution of ammonium acetate in EtOH (137 mL, prepared as follows: 150 ml of EtOH was heated to reflux, then ammonium acetate was added until saturation, followed by addition of 30% aqueous ammonium hydroxide (15.84 mL)). After 5 h, NaBH₃CN (0.09 mmol, 3 equiv) was added and the reaction mixture was heated to 100° C. for 15 min. The residue was purified by reversed phase flash chromatography (C18 silica gel column), eluting with 0-100% acetonitrile in 0.1% formic acid/water over 10 min, to provide 4-(aminomethyl)-N-(3-chloro-1H-indol-7-yl)benzenesulfonamide (I-20, 45 mg, 40.05%) as a light yellow solid. LCMS (ESI) m/z: [M+H]⁺=336.10.

### Step 5: Preparation of tert-butyl (2-((4-(N-(3-chloro-1H-indol-7-yl)sulfamoyl)benzyl)amino)-2-oxoethyl)carbamate

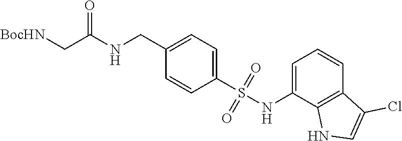 [SMILES: CC(C)(C)OC(=O)NCC(=O)NCc1ccc(S(=O)(=O)Nc2cccc3c(Cl)c[nH]c23)cc1]

To a stirred mixture of 4-(aminomethyl)-N-(3-chloro-1H-indol-7-yl)benzenesulfonamide (280.0 mg, 0.834 mmol, 1.00 equiv) and [(tert-butoxycarbonyl)amino]acetic acid (175.28 mg, 1.001 mmol, 1.20 equiv) in DMF (3.00 mL) was added HATU (412.15 mg, 1.084 mmol, 1.30 equiv) and DIEA (323.29 mg, 2.501 mmol, 3.00 equiv) in portions at room temperature. The mixture was stirred for 2 h under an atmosphere of dry nitrogen. The mixture was concentrated and the residue was purified by reversed phase flash chromatography to give tert-butyl (2-((4-(N-(3-chloro-1H-indol-7-yl)sulfamoyl)benzyl)amino)-2-oxoethyl)carbamate (172 mg, 40.17%) as a white solid. LCMS (ESI) m/z: [M+H]⁺=493.

### Step 6: Preparation of 2-amino-N-([4-[(3-chloro-1H-indol-7-yl)sulfamoyl]phenyl]methyl)acetamide (I-21)

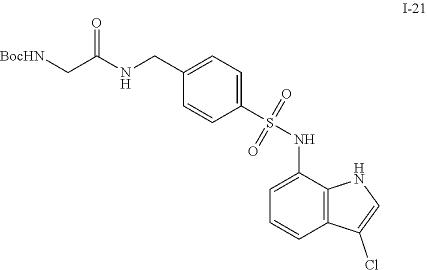 [SMILES: CC(C)(C)OC(=O)NCC(=O)NCc1ccc(S(=O)(=O)Nc2cccc3c(Cl)c[nH]c23)cc1]

A mixture of tert-butyl (2-((4-(N-(3-chloro-1H-indol-7-yl)sulfamoyl)benzyl)amino)-2-oxoethyl)carbamate (162.00 mg, 0.329 mmol, 1.00 equiv) and TFA (1.00 mL, 13.463 mmol, 40.97 equiv) in DCM (4.00 mL) was stirred at room temperature for 2 h under an atmosphere of dry nitrogen. The solution was concentrated, and the residue purified by reversed phase flash chromatography to afford 2-amino-N-([4-[(3-chloro-1H-indol-7-yl)sulfamoyl]phenyl]methyl)acetamide (I-21, 112 mg, 83.28%) as a white solid. LCMS (ESI) m/z: [M+H]⁺=393.

### Preparation of 5-[(8-aminooctyl)amino]-2-(2,6-dioxopiperidin-3-yl)isoindole-1,3-dione TFA (I-22)

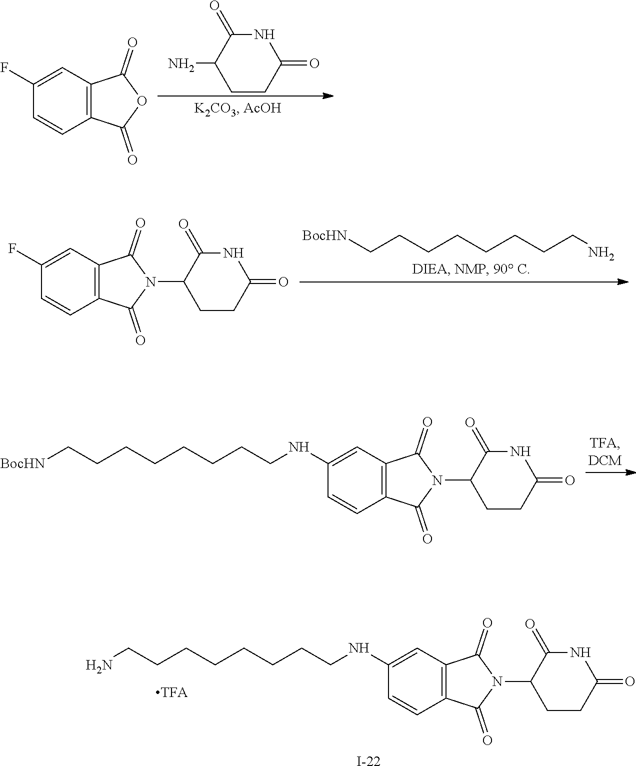

### Step 1: Preparation of 2-(2,6-dioxopiperidin-3-yl)-5-fluoro-2,3-dihydro-1H-isoindole-1,3-dione

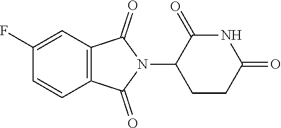 [SMILES: O=C1CCC(N2C(=O)c3ccc(F)cc3C2=O)C(=O)N1]

To a stirred mixture of 5-fluoro-1,3-dihydro-2-benzofuran-1,3-dione (5.6 g, 33.713 mmol, 1.00 equiv) in AcOH (60 mL) was added 2,6-dioxopiperidin-3-aminium chloride (5.55 g, 33.713 mmol, 1.00 equiv) and sodium acetate (5.53 g, 67.426 mmol, 2.00 equiv) at 120° C. under an atmosphere of dry nitrogen. After 14 h, the reaction mixture was concentrated under reduced pressure to remove most acetic acid. The residue was poured into water (100 mL) and stirred for 10 min. The mixture was filtered.

The filtered cake was washed with water and dried. This gave the title compound (8.03 g, 81.92%) as a pink solid. LCMS (ESI) m/z: [M+H]⁺=277.

### Step 2: Preparation of tert-butyl N-(8-[[2-(2,6-dioxopiperidin-3-yl)-1,3-dioxo-2,3-dihydro-1H-isoindol-5-yl]amino]octyl)carbamate

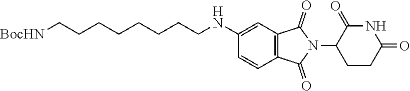 [SMILES: CC(C)(C)OC(=O)NCCCCCCCCNc1ccc2c(c1)C(=O)N(C1CCC(=O)NC1=O)C2=O]

To a stirred mixture of 2-(2,6-dioxopiperidin-3-yl)-5-fluoro-2,3-dihydro-1H-isoindole-1,3-dione (5.00 g, 18.101 mmol, 1.00 equiv) and tert-butyl N-(8-aminooctyl)carbamate (6.64 g, 27.152 mmol, 1.50 equiv) in NMP (50.00 mL) was added DIEA (7.02 g, 54.304 mmol, 3.00 equiv) at 90° C. After 3 h, to the mixture was added water (100 mL), followed by extraction with EtOAc (200 mL×3). The residue was purified by reversed phase flash chromatography with the following conditions: column, C18 silica gel; mobile phase, ACN in water, 0% to 50% gradient; detector, UV 254 nm. This gave the title compound (3.50 g, 36.69%) as a yellow solid. LCMS (ESI) m/z: [M+H]⁺=501.

### Step 3: Preparation of 5-[(8-aminooctyl)amino]-2-(2,6-dioxopiperidin-3-yl)isoindole-1,3-dione trifluoroacetate (I-22)

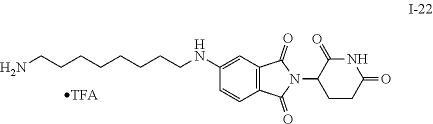 [SMILES: NCCCCCCCCNc1ccc2c(c1)C(=O)N(C1CCC(=O)NC1=O)C2=O]

To a stirred mixture of tert-butyl N-(8-[[2-(2,6-dioxopiperidin-3-yl)-1,3-dioxo-2,3-dihydro-1H-isoindol-5-yl]amino]octyl)carbamate (3.85 g, 7.691 mmol, 1.00 equiv) in DCM (9.00 ml) was added TFA (3.00 mL) at room temperature. The resulting mixture was stirred for 1 h at room temperature. The resulting mixture was concentrated under reduced pressure. The residue was purified by CombiFlash to afford I-22 (2.22 g, 56.20%) as a yellow solid. ¹H NMR (400 MHz, DMSO) δ 11.08 (s, 1H), 7.80-7.72 (m, 1H), 7.72-7.67 (m, 2H), 7.55 (dd, 1H), 7.14 (s, 1H), 6.95 (d, 1H), 6.88-6.77 (m, 1H), 5.08-4.98 (m, 1H), 3.21-3.05 (m, 1H), 2.95-2.75 (m, 2H), 2.66-2.51 (m, 2H), 2.12-1.94 (m, 1H), 1.63-1.49 (m, 3H), 1.45-1.31 (m, 4H). LCMS (ESI) m/z: [M+H]⁺=401.21.

The following intermediates in Table A5 were prepared in a similar manner as described in the preparation of I-22.

TABLE A5

| | | | LCMS |
|---|---|---|---|
| | Intermediate | | (ESI)m/z: |
| Structure | No. | Name | [M + H]⁺ |
| 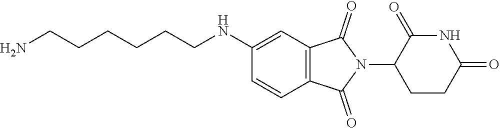 [SMILES: NCCCCCCNc1ccc2c(c1)C(=O)N(C1CCC(=O)NC1=O)C2=O] | I-23 | 5-((6-aminohexyl)amino)-2-(2,6- dioxopiperidin-3-yl)isoindoline- 1,3-dione | 373.3 |
| 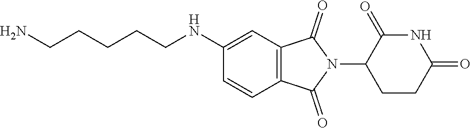 [SMILES: NCCCCCNc1ccc2c(c1)C(=O)N(C1CCC(=O)NC1=O)C2=O] | I-24 | 5-((5-aminopentyl)amino)-2- (2,6-dioxopiperidin-3- yl) isoindoline-1,3-dione | 359.15 |
| 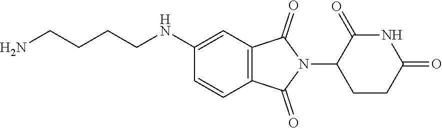 [SMILES: NCCCCNc1ccc2c(c1)C(=O)N(C1CCC(=O)NC1=O)C2=O] | I-25 | 5-((4-aminobutyl)amino)-2-(2,6- dioxopiperidin-3-yl)isoindoline- 1,3-dione | 345.15 |
| 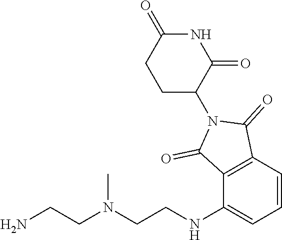 [SMILES: CN(CCN)CCNc1cccc2c1C(=O)N(C1CCC(=O)NC1=O)C2=O] | I-26 | 4-([2-[(2- aminoethyl)(methyl)amino]ethyl] amino)-2-(2,6-dioxopiperidin-3- yl) isoindole-1,3-dione | 374.15 |
| 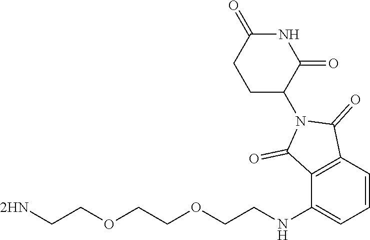 | I-27 | 4-((2-(2-(2- aminoethoxy)ethoxy)ethyl) amino)-2-(2,6-dioxopiperidin-3- yl) isoindoline-1,3-dione | 405.4 |
| 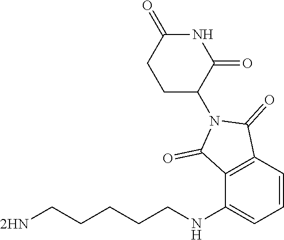 | I-28 | 4-((5-aminopentyl)amino)-2- (2,6-dioxopiperidin-3- yl) isoindoline-1,3-dione | 359.4 |

### Preparation of 4-[[2-(2,6-Dioxopiperidin-3-yl)-1,3-dioxo-2,3-dihydro-1H-isoindol-4-yl]oxy]butanoic acid (I-29)

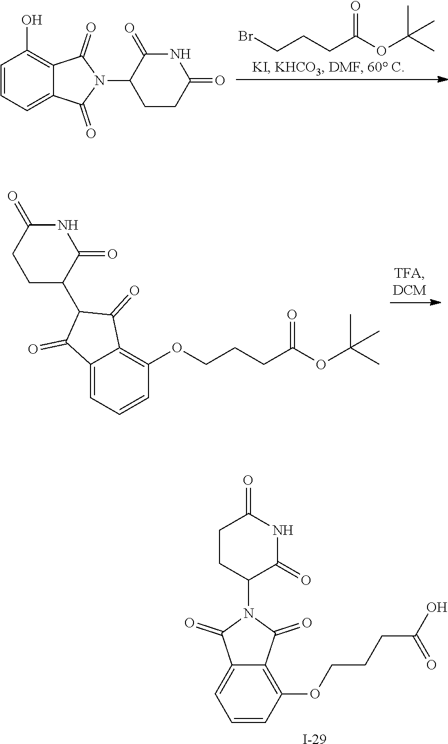

### Step 1: Preparation of tert-butyl 4-[[2-(2,6-dioxopiperidin-3-yl)-1,3-dioxoisoindol-4-yl]oxy]butanoate

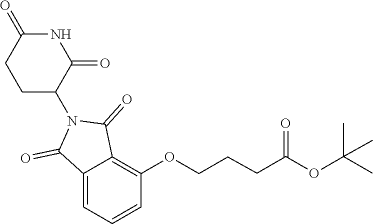 [SMILES: CC(C)(C)OC(=O)CCCOc1cccc2c1C(=O)N(C1CCC(=O)NC1=O)C2=O]

To a solution of 2-(2,6-dioxopiperidin-3-yl)-4-hydroxyisoindole-1,3-dione (2.00 g, 7.293 mmol, 1.00 equiv) and tert-butyl 4-bromobutanoate (1.95 g, 8.752 mmol, 1.2 equiv) in DMF (10.00 mL) was added KI (0.12 g, 0.729 mmol, 0.1 equiv) and KHCO₃ (1.10 g, 10.940 mmol, 1.5 equiv). The resulting solution was stirred at 60° C. for 5 h. The mixture was diluted with EtOAc (50 mL) and washed with water (50 mL×3). The organic layer was dried over anhydrous sodium sulfate, filtered and concentrated to give a crude product. The crude product was purified by flash C18 chromatography, elution gradient 0 to 32% ACN in water to give the title product (1.5 g, 49.39%) as an off-white solid. LCMS (ESI) m/z [M+H]⁺=417.

### Step 2: Preparation of 4-[[2-(2-(2,6-dioxopiperidin-3-yl)-1,3-dioxo-2,3-dihydro-1H-isoindol-4-yl]oxy]butanoic acid (I-29)

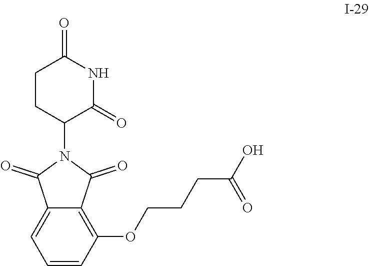 [SMILES: O=C(O)CCCOc1cccc2c1C(=O)N(C1CCC(=O)NC1=O)C2=O]

To a stirred solution of tert-butyl 4-[[2-(2,6-dioxopiperidin-3-yl)-1,3-dioxoisoindol-4-yl]oxy]butanoate (450 mg, 1.081 mmol, 1 equiv) in DCM (5 ml) was added TFA (1 mL). The resulting solution was stirred for 2 h at 25° C. The resulting mixture was concentrated. This provided I-29 (360 mg, 92.46%) as a white solid. ¹H NMR (400 MHz, Methanol-d4) δ 7.79 (t, J=8.4, 7.4 Hz, 1H), 7.47 (d, J=7.8 Hz, 2H), 5.12 (dd, J=12.6, 5.5 Hz, 1H), 4.30 (t, J=6.2 Hz, 2H), 2.95-2.66 (m, 3H), 2.60 (t, J=7.3 Hz, 2H), 2.25-2.18 (m, 3H). LCMS (ESI) m/z: [M+H]⁺=361.10.

The following intermediates in Table A6 were prepared in a similar manner as described in the preparation of intermediate I-29.

TABLE A6

| | | | LCMS |
|---|---|---|---|
| | Intermediate | | (ESI)m/z: |
| Structure | No. | Name | [M + H]⁺ |
| 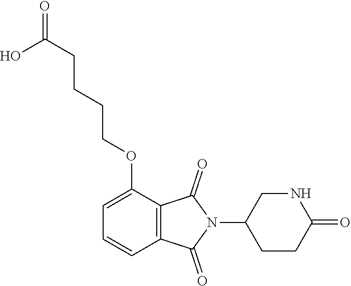 [SMILES: O=C(O)CCCCOc1cccc2c1C(=O)N(C1CCC(=O)NC1)C2=O] | I-30 | 5-((2-(2,6-dioxopiperidin-3-yl)- 1,3-dioxoisoindolin-4- yl)oxy)pentanoic acid | 375.1 |
| 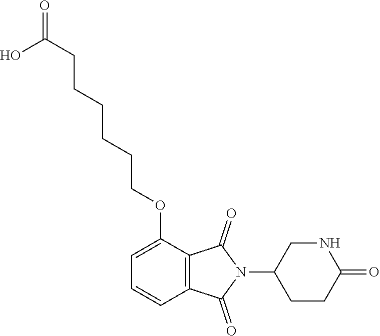 [SMILES: O=C(O)CCCCCCOc1cccc2c1C(=O)N(C1CCC(=O)NC1)C2=O] | I-31 | 7-((2-(2,6-dioxopiperidin-3-yl)- 1,3-dioxoisoindolin-4- yl)oxy)heptanoic acid | 403.1 |
| 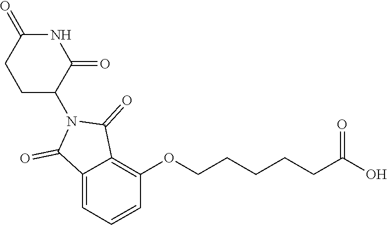 [SMILES: O=C(O)CCCCCOc1cccc2c1C(=O)N(C1CCC(=O)NC1=O)C2=O] | I-32 | 6-((2-(2,6-dioxopiperidin-3-yl)- 1,3-dioxoisoindolin-4- yl)oxy)hexanoic acid | 389.4 |
| 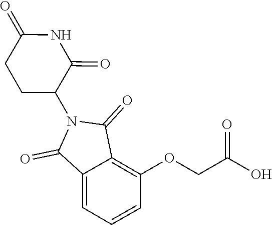 [SMILES: O=C(O)COc1cccc2c1C(=O)N(C1CCC(=O)NC1=O)C2=O] | I-33 | 2-((2-(2,6-dioxopiperidin-3-yl)- 1,3-dioxoisoindolin-4- yl)oxy)acetic acid | 333.2 |

### Preparation of 3-(2-(2-((2-(2,6-dioxopiperidin-3-yl)-1,3-dioxoisoindolin-4-yl)amino)ethoxy)ethoxy)propanoic acid (I-34)

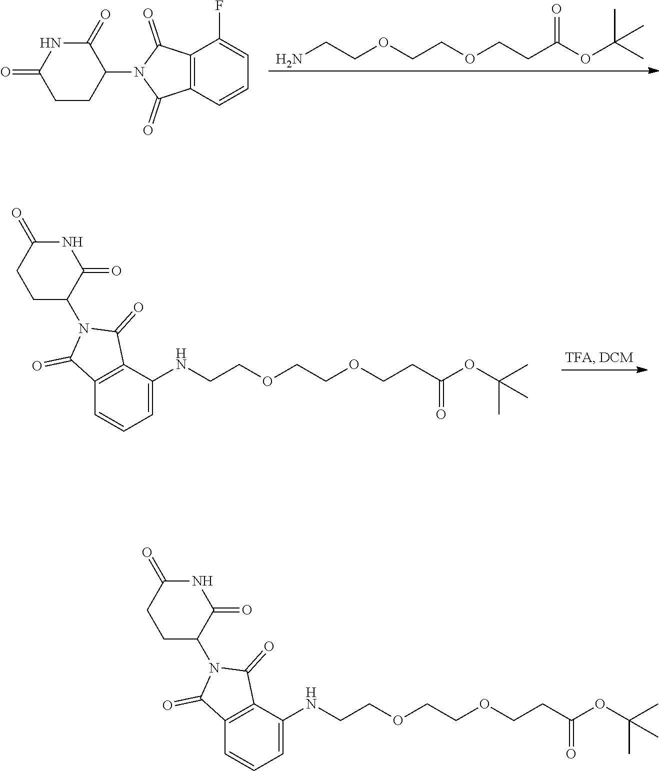 [SMILES: O=C1CCC(N2C(=O)c3cccc(F)c3C2=O)C(=O)N1 ; CC(C)(C)OC(=O)CCOCCOCCN ; CC(C)(C)OC(=O)CCOCCOCCNc1cccc2c1C(=O)N(C1CCC(=O)NC1=O)C2=O ; CC(C)(C)OC(=O)CCOCCOCCNc1cccc2c1C(=O)N(C1CCC(=O)NC1=O)C2=O]

### Step 1: Preparation of tert-butyl 3-(2-(2-((2-(2,6-dioxopiperidin-3-yl)-1,3-dioxoisoindolin-4-yl)amino)ethoxy)ethoxy)propanoate

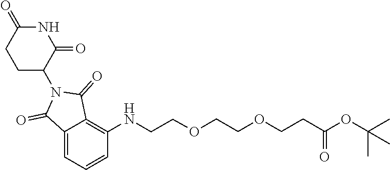 [SMILES: CC(C)(C)OC(=O)CCOCCOCCNc1cccc2c1C(=O)N(C1CCC(=O)NC1=O)C2=O]

To a solution of 2-(2,6-dioxopiperidin-3-yl)-4-fluoro-2,3-dihydro-1H-isoindole-1,3-dione (1.00 g, 3.620 mmol, 1.00 equiv) in NMP (10.00 mL) was added tert-butyl 3-[2-(2-aminoethoxy)ethoxy]propanoate (929.10 mg, 3.982 mmol, 1.10 equiv). The resulting mixture was stirred overnight at 90° C. The mixture was allowed to cool down to room temperature. The resulting mixture was diluted with EtOAc (30 mL). The organic layer was washed with water (10 mL×5), and then brine (20 mL). The resulting mixture was concentrated under reduced pressure. The residue was purified by silica gel column chromatography, eluted with Petroleum ether/EtOAc (from 5:1 to 1:1) to afford tert-butyl 3-(2-(2-((2-(2,6-dioxopiperidin-3-yl)-1,3-dioxoisoindolin-4-yl)amino)ethoxy)ethoxy)propanoate (1.14 g, 64.33%) as a yellow solid. LCMS (ESI) m/z: [M+H]⁺=490.

### Step 2: Preparation of 3-(2-(2-((2-(2,6-dioxopiperidin-3-yl)-1,3-dioxoisoindolin-4-yl)amino)ethoxy)ethoxy)propanoic acid (I-34)

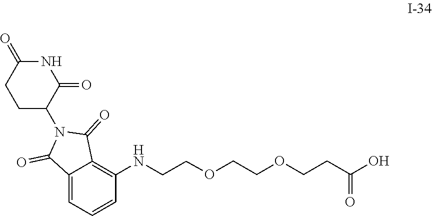 [SMILES: O=C(O)CCOCCOCCNc1cccc2c1C(=O)N(C1CCC(=O)NC1=O)C2=O]

To a stirred solution of tert-butyl 3-(2-(2-((2-(2,6-dioxopiperidin-3-yl)-1,3-dioxoisoindolin-4-yl)amino)ethoxy)ethoxy)propanoate (1.14 g, 2.329 mmol, 1.00 equiv) in DCM (10.00 ml) was added TFA (0.52 mL, 4.551 mmol, 3.00 equiv) dropwise at room temperature. The resulting mixture was stirred for 2 h at room temperature. The resulting mixture was concentrated under vacuum and the residue was purified by reversed phase flash chromatography with the following conditions: column, C18 spherical column; mobile phase, ACN in water, 0% to 100% gradient in 50 min; 70 mL/min detector, UV 254 nm to provide I-34 (896 mg, 70.28%) as a yellow solid. ¹H NMR (400 MHz, DMSO) δ 12.15 (s, 1H), 11.09 (s, 1H), 7.63-7.55 (m, 1H), 7.15 (d, 1H), 7.05 (d, 1H), 6.61 (t, 1H), 5.06 (dd, 1H), 3.65-3.44 (m, 8H), 2.87 (d, 1H), 2.59 (d, 2H), 2.43 (t, 2H), 2.04 (m, 1H); LCMS (ESI) m/z: [M+H]⁺=434.15.

The following intermediates in Table A7 were prepared in a similar manner as described in the preparation of intermediate I-34.

TABLE A7

| | | | LCMS |
|---|---|---|---|
| | Intermediate | | (ESI)m/z: |
| Structure | No. | Name | [M + H]⁺ |
| 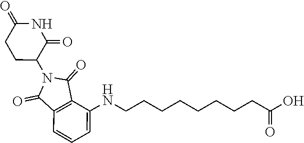 [SMILES: O=C(O)CCCCCCCCNc1cccc2c1C(=O)N(C1CCC(=O)NC1=O)C2=O] | I-35 | 9-[[2-(2,6-dioxopiperidin-3- yl)-1,3-dioxo-2,3-dihydro-1H- isoindol-4-yl]amino]nonanoic acid | 430.19 |
| 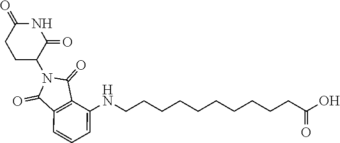 [SMILES: O=C(O)CCCCCCCCCCNc1cccc2c1C(=O)N(C1CCC(=O)NC1=O)C2=O] | I-36 | 11-((2-(2,6-dioxopiperidin-3- yl)-1,3-dioxoisoindolin-4- yl)amino)undecanoic acid | 458.3 |
| 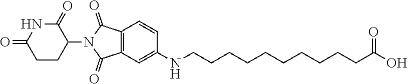 [SMILES: O=C(O)CCCCCCCCCCNc1ccc2c(c1)C(=O)N(C1CCC(=O)NC1=O)C2=O] | I-37 | 11-((2-(2,6-dioxopiperidin-3- yl)-1,3-dioxoisoindolin-5- yl)amino)undecanoic acid | 458.4 |
| 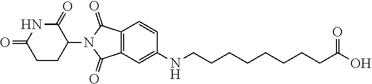 [SMILES: O=C(O)CCCCCCCCNc1ccc2c(c1)C(=O)N(C1CCC(=O)NC1=O)C2=O] | I-38 | 9-((2-(2,6-dioxopiperidin-3- yl)-1,3-dioxoisoindolin-5- yl)amino)nonanoic acid | 430.2 |
| 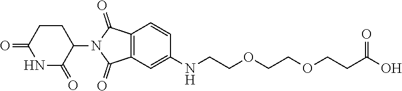 [SMILES: O=C(O)CCOCCOCCNc1ccc2c(c1)C(=O)N(C1CCC(=O)NC1=O)C2=O] | I-39 | 3-(2-(2-((2-(2,6- dioxopiperidin-3-yl)-1,3- dioxoisoindolin-5- yl)amino)ethoxy)ethoxy)prop anoic acid | 434.2 |
| 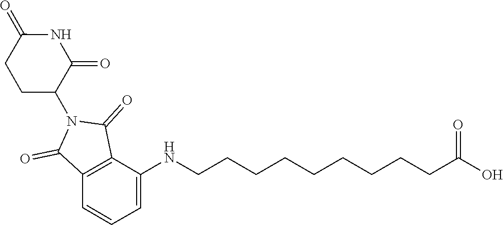 [SMILES: O=C(O)CCCCCCCCCNc1cccc2c1C(=O)N(C1CCC(=O)NC1=O)C2=O] | I-40 | 10-((2-(2,6-dioxopiperidin-3- yl)-1,3-dioxoisoindolin-4- yl)amino)decanoic acid | 444.25 |
| 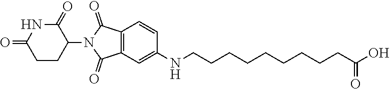 [SMILES: O=C(O)CCCCCCCCCNc1ccc2c(c1)C(=O)N(C1CCC(=O)NC1=O)C2=O] | I-41 | 10-((2-(2,6-dioxopiperidin-3- yl)-1,3-dioxoisoindolin-5- yl)amino)decanoic acid | 444.30 |
| 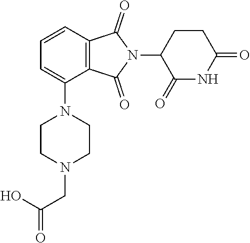 [SMILES: O=C(O)CN1CCN(c2cccc3c2C(=O)N(C2CCC(=O)NC2=O)C3=O)CC1] | K-18 | 2-(4-(2-(2,6-dioxopiperidin-3- yl)-1,3-dioxoisoindolin-4- yl)piperazin-1-yl)acetic acid | 401 |

### Preparation of 4-((3-(2,6-dioxopiperidin-3-yl)-4-oxo-3,4-dihydrobenzo[d][1,2,3]triazin-5-yl)amino)butanoic acid (K-19)

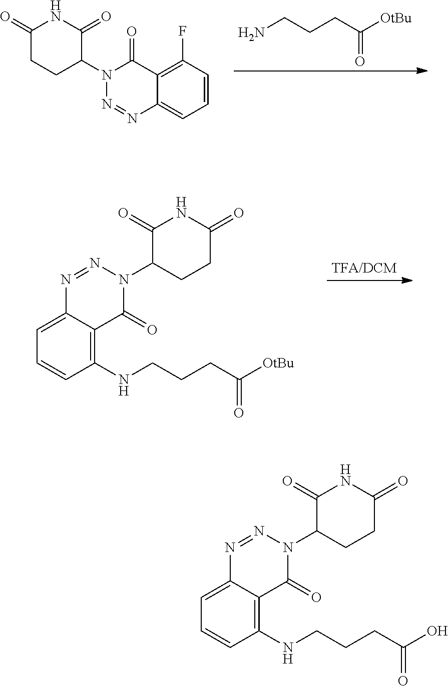 [SMILES: O=C1CCC(n2nnc3cccc(F)c3c2=O)C(=O)N1 ; CC(C)(C)OC(=O)CCCN ; CC(C)(C)OC(=O)CCCNc1cccc2nnn(C3CCC(=O)NC3=O)c(=O)c12 ; O=C(O)CCCNc1cccc2nnn(C3CCC(=O)NC3=O)c(=O)c12]

### Step 1: Preparation of tert-butyl 4-((3-(2,6-dioxopiperidin-3-yl)-4-oxo-3,4-dihydrobenzo[d][1,2,3]triazin-5-yl)amino)butanoate

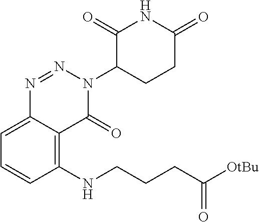 [SMILES: CC(C)(C)OC(=O)CCCNc1cccc2nnn(C3CCC(=O)NC3=O)c(=O)c12]

Into a 8-mL sealed tube was placed 3-(5-fluoro-4-oxo-1,2,3-benzotriazin-3-yl)piperidine-2,6-dione (100.00 mg, 0.362 mmol, 1.00 equiv), NMP (1.50 mL), tert-butyl 4-aminobutanoate (86.47 mg, 0.543 mmol, 1.5 equiv), and DIEA (140.37 mg, 1.086 mmol, 3 equiv). The resulting solution was stirred for 5 h at 90 degrees C. The reaction mixture was purified by reverse flash chromatography with the following conditions: column, C18 silica gel; mobile phase, MeCN in water, 10% to 50% gradient over 10 min; detector, UV 254 nm. This resulted in 100 mg (66.49%) of tert-butyl 4-((3-(2,6-dioxopiperidin-3-yl)-4-oxo-3,4-dihydrobenzo[d][1,2,3]triazin-5-yl)amino)butanoate as a yellow solid. LCMS (ESI) m/z: [M+H]⁺=416.

### Step 2: Preparation of 4-((3-(2,6-dioxopiperidin-3-yl)-4-oxo-3,4-dihydrobenzo[d][1,2,3]triazin-5-yl)amino)butanoic acid (K-19)

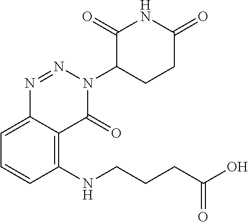 [SMILES: O=C(O)CCCNc1cccc2nnn(C3CCC(=O)NC3=O)c(=O)c12]

Into a 8-mL sealed tube, was placed tert-butyl 4-((3-(2,6-dioxopiperidin-3-yl)-4-oxo-3,4-dihydrobenzo[d][1,2,3]triazin-5-yl)amino)butanoate (90.00 mg, 0.217 mmol, 1.00 equiv), DCM (5.00 mL), and TFA (0.50 mL). The resulting solution was stirred for 30 min at room temperature, then concentrated under reduced pressure to afford 4-((3-(2,6-dioxopiperidin-3-yl)-4-oxo-3,4-dihydrobenzo[d][1,2,3]triazin-5-yl)amino)butanoic acid (70 mg, quant.) as a light yellow crude solid. The crude product was used in the next step directly without further purification. LCMS (ESI) m/z: [M+H]⁺=360.

The following intermediates in Table A8 were prepared in a similar manner as described in the preparation of intermediate K-19.

TABLE A8

| | | | LCMS |
|---|---|---|---|
| | Intermediate | | (ESI)m/z: |
| Structure | No. | Name | [M + H]⁺ |
| 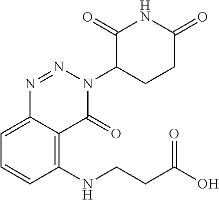 [SMILES: O=C(O)CCNc1cccc2nnn(C3CCC(=O)NC3=O)c(=O)c12] | K-20 | 3-((3-(2,6-dioxopiperidin-3- yl)-4-oxo-3,4- dihydrobenzo[d][1,2,3]triazin- 5-yl)amino)propanoic acid | 346 |
| 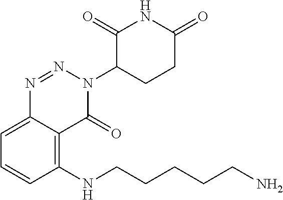 [SMILES: NCCCCCNc1cccc2nnn(C3CCC(=O)NC3=O)c(=O)c12] | K-21 | 3-[5-[(5-aminopentyl)amino]- 4-oxo-1,2,3-benzotriazin-3- yl]piperidine-2, 6-dione | 359 |

### Preparation of 5-((3-(2,6-dioxopiperidin-3-yl)-4-oxo-3,4-dihydrobenzo[d] [1,2,3] triazin-6-yl)amino)pentanoic acid (K-22)

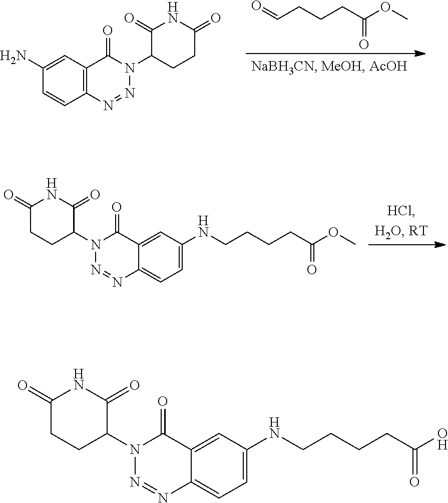

### Step 1: Preparation of methyl 5-((3-(2,6-dioxopiperidin-3-yl)-4-oxo-3,4-dihydrobenzo[d][1,2,3] triazin-6-yl)amino)pentanoate

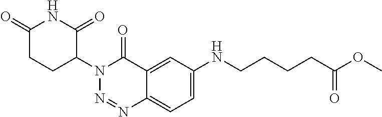 [SMILES: COC(=O)CCCCNc1ccc2nnn(C3CCC(=O)NC3=O)c(=O)c2c1]

To a stirred solution of 3-(6-amino-4-oxo-1,2,3-benzotriazin-3-yl) piperidine-2,6-dione (120.00 mg, 0.439 mmol, 1.00 equiv) and methyl 5-oxopentanoate (57.15 mg, 0.439 mmol, 1.00 equiv) in MeOH (2.00 mL) was added AcOH (0.20 mL) in portions at room temperature. The resulting mixture was stirred for 2 h at 50 degrees C. To the above mixture was added NaBH₃CN (82.79 mg, 1.317 mmol, 3.00 equiv) in portions over 1 min. The resulting mixture was stirred for additional 2 h at 50 degrees C. The resulting mixture was diluted with water (20 mL). The aqueous layer was extracted with CH₂Cl₂ (4×20 mL). The resulting mixture was concentrated under reduced pressure to provide methyl 5-((3-(2,6-dioxopiperidin-3-yl)-4-oxo-3,4-dihydrobenzo[d] [1,2,3] triazin-6-yl)amino)pentanoate which was used in the next step directly without further purification. LCMS (ESI) m/z [M+H]⁺=388.15.

### Step 2: Preparation of 5-((3-(2,6-dioxopiperidin-3-yl)-4-oxo-3,4-dihydrobenzo[d] [1,2,3] triazin-6-yl)amino)pentanoic acid (K-22)

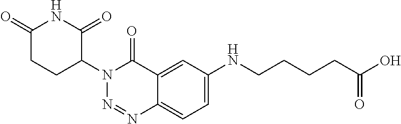 [SMILES: O=C(O)CCCCNc1ccc2nnn(C3CCC(=O)NC3=O)c(=O)c2c1]

To a 25 mL vial was added methyl 5-((3-(2,6-dioxopiperidin-3-yl)-4-oxo-3,4-dihydrobenzo[d] [1,2,3]triazin-6-yl)amino)pentanoate (120.00 mg, 0.310 mmol, 1.00 equiv) and HCl (6 M, 5.00 mL, 0.027 mmol, 5.00 equiv) at room temperature. The resulting mixture was stirred for 1 h at room temperature under air atmosphere, then concentrated under reduced pressure, to provide crude 5-((3-(2,6-dioxopiperidin-3-yl)-4-oxo-3,4-dihydrobenzo[d] [1,2,3] triazin-6-yl)amino)pentanoic acid which was used in the next step directly without further purification. LCMS (ESI) m/z [M+H]⁺=374.10.

### Preparation of 2-[(2-[[2-(2,6-dioxopiperidin-3-yl)-1,3-dioxoisoindol-5-yl]oxy]acetamido) methyl]cyclopropane-1-carboxylic acid (I-42)

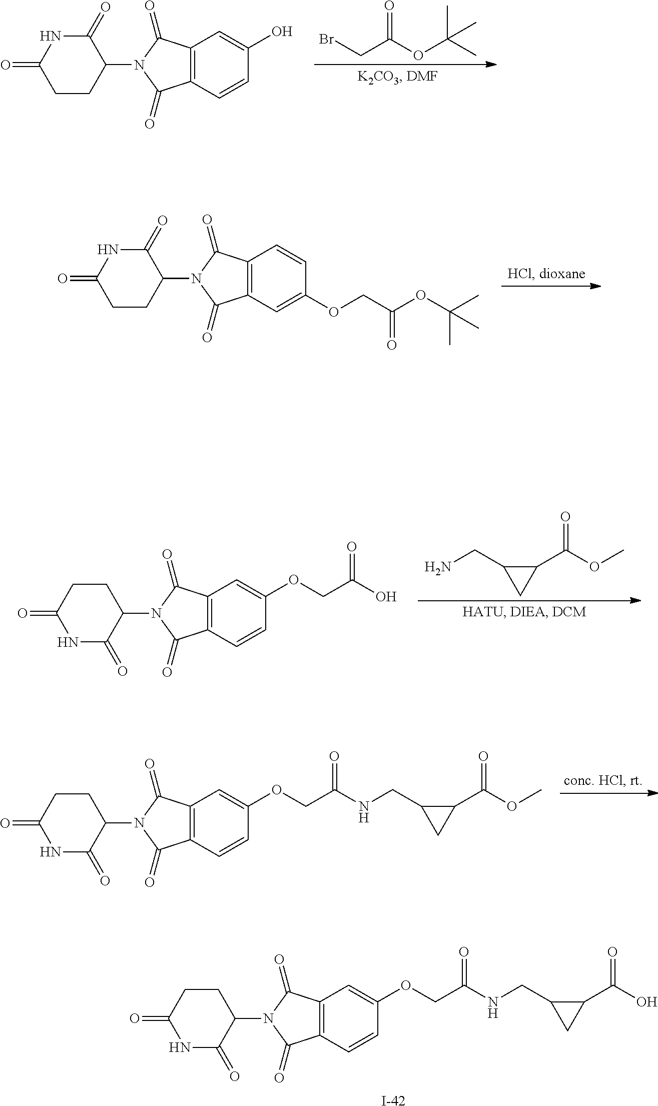

### Step 1: Preparation of tert-butyl 2-[[2-(2,6-dioxopiperidin-3-yl)-1,3-dioxoisoindol-5-yl]oxy] acetate

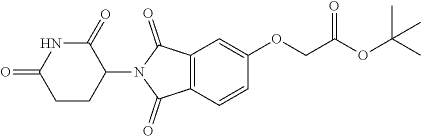 [SMILES: CC(C)(C)OC(=O)COc1ccc2c(c1)C(=O)N(C1CCC(=O)NC1=O)C2=O]

To a stirred solution of 2-(2,6-dioxopiperidin-3-yl)-5-hydroxyisoindole-1,3-dione (5.50 g, 20.056 mmol, 1.00 equiv) and tert-butyl 2-bromoacetate (3.91 g, 20.056 mmol, 1.00 equiv) in DMF (15.00 mL) was added K2CO3 (8.32 g, 60.168 mmol, 3 equiv). The resulting mixture stirred overnight at room temperature, then taken up in water, extracted with EtOAc (3×200 mL), and concentrated. The residue was purified by reversed phase flash chromatography with the following conditions: column, C18 silica gel; mobile phase, ACN in water, 10% to 50% gradient in 10 min; detector, UV 254 nm. This resulted in tert-butyl 2-[[2-(2,6-dioxopiperidin-3-yl)-1,3-dioxoisoindol-5-yl]oxy] acetate (3.2 g, 45.19%) as an off-white solid. LCMS (ESI) m/z: [M+H]⁺=389.

### Step 2: Preparation of [[2-(2,6-dioxopiperidin-3-yl)-1,3-dioxoisoindol-5-yl]oxy]acetic acid

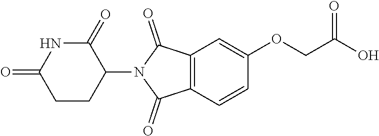 [SMILES: O=C(O)COc1ccc2c(c1)C(=O)N(C1CCC(=O)NC1=O)C2=O]

A solution of tert-butyl 2-[[2-(2,6-dioxopiperidin-3-yl)-1,3-dioxoisoindol-5-yl]oxy] acetate (3.20 g, 8.239 mmol, 1.00 equiv) and dry HCl in dioxane (15.00 mL, 493.678 mmol, 59.92 equiv) was stirred for 2 h at room temperature. The resulting mixture was concentrated under reduced pressure. This provided the title compound (1.12 g) as a yellow solid. LCMS (ESI) m/z: [M+H]⁺=289.

### Step 3: Preparation of methyl 2-[(2-[[2-(2,6-dioxopiperidin-3-yl)-1,3-dioxoisoindol-5-yl]oxy]acetamido) methyl]cyclopropane-1-carboxylate

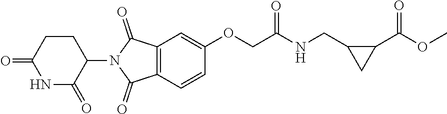 [SMILES: COC(=O)C1CC1CNC(=O)COc1ccc2c(c1)C(=O)N(C1CCC(=O)NC1=O)C2=O]

To a stirred solution of [[2-(2,6-dioxopiperidin-3-yl)-1,3-dioxoisoindol-5-yl]oxy]acetic acid (670.00 mg, 2.016 mmol, 1.00 equiv) and methyl 2-(aminomethyl)cyclopropane-1-carboxylate (260.44 mg, 2.016 mmol, 1.00 equiv) in DMF (15.00 mL) was added HATU (1150.07 mg, 3.025 mmol, 1.50 equiv) and DIEA (781.84 mg, 6.049 mmol, 3.00 equiv) dropwise at room temperature over 2 h. The resulting mixture was concentrated under reduced pressure. The residue was purified by reversed phase flash chromatography with the following conditions: column, C18 silica gel; mobile phase, ACN in water, 10% to 50% gradient in 10 min; detector, UV 254 nm. This resulted in methyl 2-[(2-[[2-(2,6-dioxopiperidin-3-yl)-1,3-dioxoisoindol-5-yl]oxy]acetamido) methyl]cyclopropane-1-carboxylate (778.6 mg, 78.37%) as a yellow oil. LCMS (ESI) m/z: [M+H]⁺=444.

### Step 4: Preparation of 2-[(2-[[2-(2,6-dioxopiperidin-3-yl)-1,3-dioxoisoindol-5-yl]oxy]acetamido)methyl]cyclopropane-1-carboxylic acid (I-42)

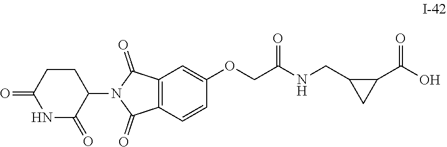 [SMILES: O=C(COc1ccc2c(c1)C(=O)N(C1CCC(=O)NC1=O)C2=O)NCC1CC1C(=O)O]

A mixture of methyl 2-[(2-[[2-(2,6-dioxopiperidin-3-yl)-1,3-dioxoisoindol-5-yl]oxy]acetamido) methyl]cyclopropane-1-carboxylate (763.90 mg, 1.00 equiv) and 4 N dry HCl in dioxane (5.00 mL) was stirred for 2 h at room temperature. The resulting mixture was concentrated under reduced pressure. The residue was purified by reversed phase flash chromatography with the following conditions: column, C18 silica gel; mobile phase, ACN in water, 10% to 50% gradient in 10 min; detector, UV 254 nm. This provided I-42 (338.2 mg) as a yellow solid. ¹H NMR (400 MHz, DMSO) δ 12.17 (s, 1H), 11.12 (s, 1H), 8.33 (t, 1H), 7.88 (d, 1H), 7.46 (d, 1H), 7.39 (dd, 1H), 5.13 (dd, 1H), 4.74 (s, 2H), 2.96-2.83 (m, 1H), 2.65-2.52 (m, 1H), 2.08 (s, 4H), 1.66 (td, 1H), 1.48 (h, 1H), 1.02 (td, 1H), 0.85 (dt, 1H). LCMS (ESI) m/z: [M+H]⁺=430.05.

### Preparation of methyl 2-[(2-[[2-(2,6-dioxopiperidin-3-yl)-1,3-dioxoisoindol-4-yl]oxy]acetamido) methyl]cyclopropane-1-carboxylic acid (I-43)

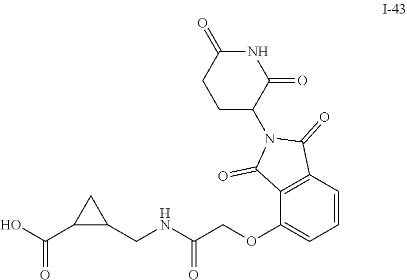 [SMILES: O=C(COc1cccc2c1C(=O)N(C1CCC(=O)NC1=O)C2=O)NCC1CC1C(=O)O]

I-43 (423 mg, 35.74%) as a white solid was prepared in a similar manner as described in the preparation of I-42. ¹H NMR (300 MHz, DMSO) δ 12.16 (s, 1H), 11.12 (s, 1H), 8.10 (s, 1H), 7.82 (t, 1H), 7.51 (d, 1H), 7.41 (d, 1H), 5.17-5.07 (m, 1H), 4.80 (s, 2H), 2.60 (d, 2H), 2.08 (s, 3H), 1.70-1.60 (m, 1H), 1.47 (d, 1H), 1.03 (d, 1H), 0.84 (d, 1H). LCMS (ESI) m/z: [M+H]⁺=430.12.

### Preparation of 7-[[(2S)-1-[(2S,4R)-4-hydroxy-2-([[4-(4-methyl-1,3-thiazol-5-yl)phenyl]methyl]carbamoyl)pyrrolidin-1-yl]-3,3-dimethyl-1-oxobutan-2-yl]carbamoyl]heptanoic acid (I-44)

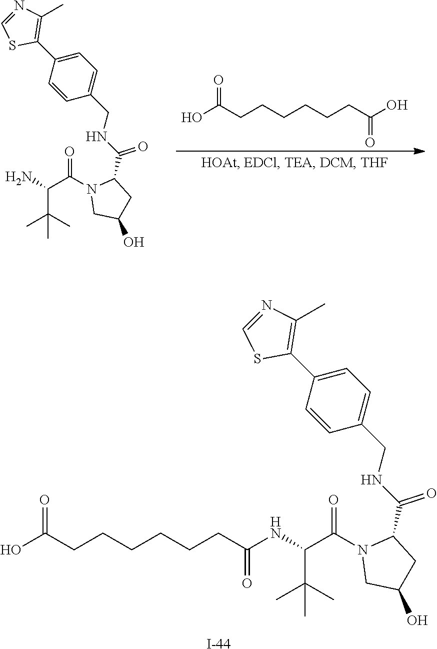

To a stirred solution of octanedioic acid (2.02 g, 11.596 mmol, 4.99 equiv) in DCM (25.00 mL) and THF (25.00 mL) were added of (2S,4R)-1-(2-amino-3,3-dimethyl-butanoyl)-4-hydroxy-N-[[4-(4-methylthiazol-5-yl)phenyl]methyl]pyrrolidine-2-carboxamide (1.00 g, 2.323 mmol, 1.00 equiv) and TEA (822.55 mg, 8.129 mmol, 3.50 equiv) and HOAt (347.73 mg, 2.555 mmol, 1.10 equiv) and EDCI (489.75 mg, 2.555 mmol, 1.10 equiv) at 0° C. The resulting solution was stirred for 2 h at 0° C. Reaction was completed as confirmed by LCMS and the resulting mixture was concentrated under reduced pressure. The residue was purified by reverse phase flash to afford I-44 (900 mg, 66.04%) as a white solid. LCMS (ESI) m/z: [M+H]⁺=587.

### Preparation of 3-(2-(3-(((S)-1-((2S,4R)-4-hydroxy-2-((4-(4-methylthiazol-5-yl)benzyl)carbamoyl) pyrrolidin-1-yl)-3,3-dimethyl-1-oxobutan-2-yl)amino)-3-oxopropoxy)ethoxy)propanoic acid (I-45)

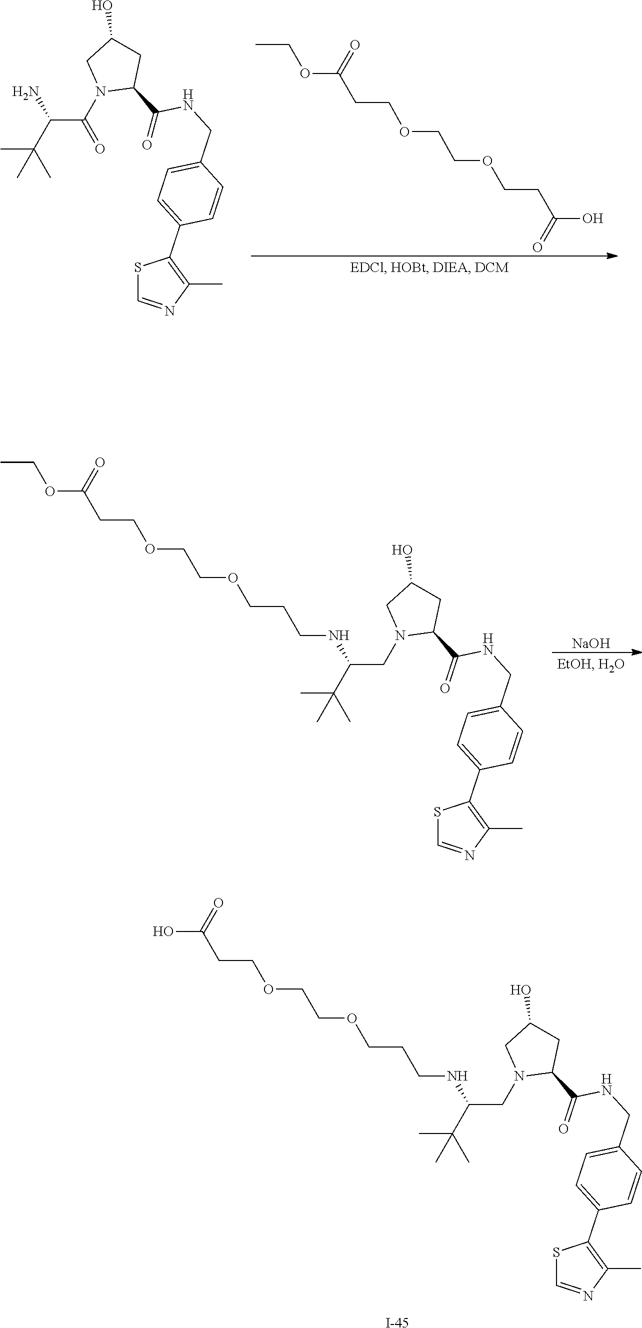

### Step 1: Preparation of ethyl 3-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)propanoate

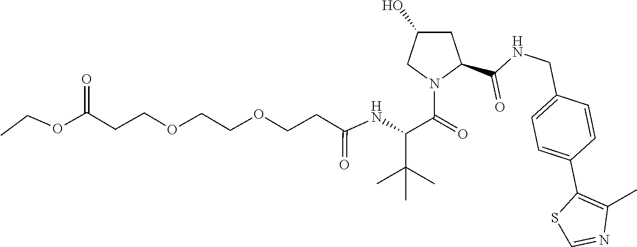 [SMILES: CCOC(=O)CCOCCOCCC(=O)N[C@H](C(=O)N1C[C@H](O)C[C@H]1C(=O)NCc1ccc(-c2scnc2C)cc1)C(C)(C)C]

To a solution of (2S,4R)-1-(2-amino-3,3-dimethyl-butanoyl)-4-hydroxy-N-[[4-(4-methylthiazol-5-yl)phenyl]methyl]pyrrolidine-2-carboxamide (500 mg, 1.07 mmol, HCl), 3-[2-(3-ethoxy-3-oxo-propoxy)ethoxy]propanoic acid (250.79 mg, 1.07 mmol, 250.79 μL), and DIEA (691.84 mg, 5.35 mmol, 932.40 μL) in DCM (5 mL) was added EDCI (246.29 mg, 1.28 mmol) and HOBt (173.60 mg, 1.28 mmol). The mixture was stirred at 20° C. for 16 h. The mixture was concentrated under reduced pressure to give a residue. The residue was purified by reversed-phase HPLC (ACN/0.1% formic acid in water). The solution was lyophilized to give ethyl 3-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)propanoate (500 mg, 70.00%) as a yellow oil. LCMS (ESI) m/z: [M+H]⁺=647.6.

### Step 2: Preparation of 3-(2-(3-(((S)-1-((2S,4R)-4-hydroxy-2-((4-(4-methylthiazol-5-yl)benzyl)carbamoyl) pyrrolidin-1-yl)-3,3-dimethyl-1-oxobutan-2-yl)amino)-3-oxopropoxy)ethoxy)propanoic acid (I-45)

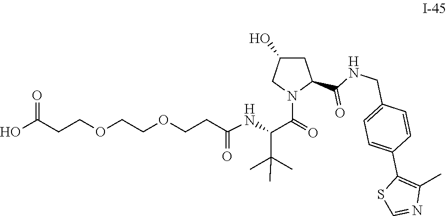 [SMILES: Cc1ncsc1-c1ccc(CNC(=O)[C@@H]2C[C@@H](O)CN2C(=O)[C@@H](NC(=O)CCOCCOCCC(=O)O)C(C)(C)C)cc1]

To a solution of give ethyl 3-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)propanoate (500 mg, 773.05 μm) in EtOH (5 mL) was added a solution of NaOH (77.30 mg, 1.93 mmol) in water (1 mL). The mixture was stirred at 20° C. for 1 h. The solution was diluted with water (30 mL), adjusted to pH 6 with 1N HCl and extracted with EtOAc (30 mL×3). The combined organic layers were dried over Na₂SO₄ and concentrated to give I-45 (470 mg, 95.02%) as a brown solid which was used into the next step without purification. LCMS (ESI) m/z: [M+Na]⁺=641.2.

The following intermediates in Table A9 were prepared in a similar manner as described in the preparation of I-45.

TABLE A9

| | | | LCMS |
|---|---|---|---|
| | Intermediate | | (ESI)m/z: |
| Structure | No. | Name | [M + H]⁺ |
| 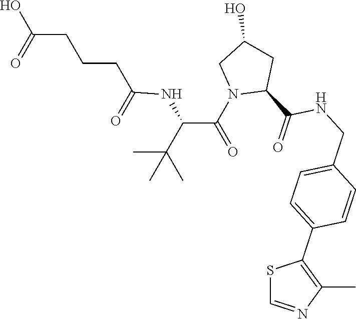 [SMILES: Cc1ncsc1-c1ccc(CNC(=O)[C@@H]2C[C@@H](O)CN2C(=O)[C@@H](NC(=O)CCCC(=O)O)C(C)(C)C)cc1] | I-46 | 5-[[(1S)-1-[(2S,4R)-4-hydroxy- 2-[[4-(4-methylthiazol-5- yl)phenyl]methylcarbamoyl] pyrrolidine-1-carbonyl]-2,2- dimethyl-propyl]amino]-5-oxo- pentanoic acid | 567.4 |
| 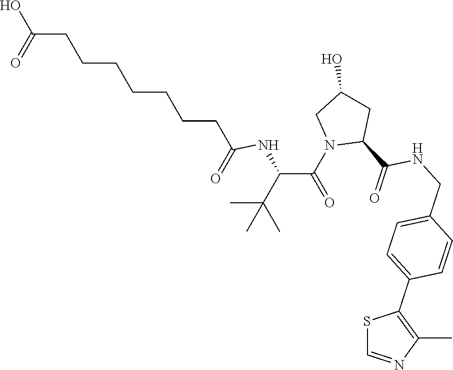 [SMILES: Cc1ncsc1-c1ccc(CNC(=O)[C@@H]2C[C@@H](O)CN2C(=O)[C@@H](NC(=O)CCCCCCCC(=O)O)C(C)(C)C)cc1] | I-47 | 9-(((S)-1-((2S,4R)-4-hydroxy- 2-((4-(4-methylthiazol-5- yl)benzyl)carbamoyl)pyrrolidin- 1-yl)-3,3-dimethyl-1-oxobutan- 2-yl)amino)-9-oxononanoic acid | 601.4 |
| 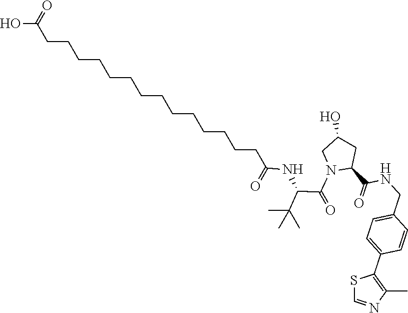 [SMILES: Cc1ncsc1-c1ccc(CNC(=O)[C@@H]2C[C@@H](O)CN2C(=O)[C@@H](NC(=O)CCCCCCCCCCCCCCC(=O)O)C(C)(C)C)cc1] | I-48 | 16-(((S)-1-((2S,4R)-4-hydroxy- 2-((4-(4-methylthiazol-5- yl)benzyl)carbamoyl) pyrrolidin-1-yl)-3,3-dimethyl-1- oxobutan-2-yl)amino)-16- oxohexadecanoic acid | 699.6 |
| 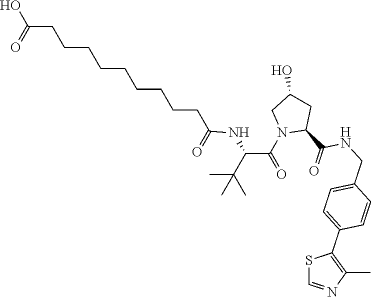 [SMILES: Cc1ncsc1-c1ccc(CNC(=O)[C@@H]2C[C@@H](O)CN2C(=O)[C@@H](NC(=O)CCCCCCCCCC(=O)O)C(C)(C)C)cc1] | I-49 | N-(2-((4-(3-(3- methoxyazetidin-1- yl)phenyl)thiazol-2-yl)amino)- 2-oxoethyl)-1-(methylsulfonyl)- 1H-pyrrole-3-carboxamide | 651.4 |
| 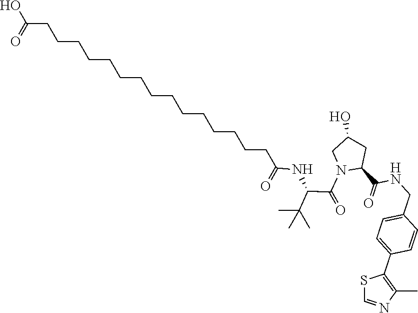 [SMILES: Cc1ncsc1-c1ccc(CNC(=O)[C@@H]2C[C@@H](O)CN2C(=O)[C@@H](NC(=O)CCCCCCCCCCCCCCCC(=O)O)C(C)(C)C)cc1] | I-50 | 17-(((S)-1-((2S,4R)-4-hydroxy- 2-((4-(4-methylthiazol-5- yl)benzyl)carbamoyl) pyrrolidin-1-yl)-3,3-dimethyl-1- oxobutan-2-yl)amino)-17- oxoheptadecanoic acid | 713.5 |
| 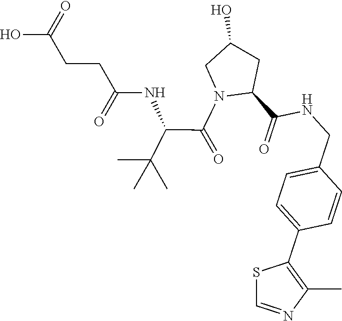 [SMILES: Cc1ncsc1-c1ccc(CNC(=O)[C@@H]2C[C@@H](O)CN2C(=O)[C@@H](NC(=O)CCC(=O)O)C(C)(C)C)cc1] | I-51 | 4-[[(1S)-1-[(2S,4R)-4-hydroxy- 2-[[4-(4-methylthiazol-5- yl)phenyl]methyl carbamoyl]pyrrolidine-1- carbonyl]-2,2-dimethyl- propyl]amino]-4-oxobutanoic acid | 531.2 |
| 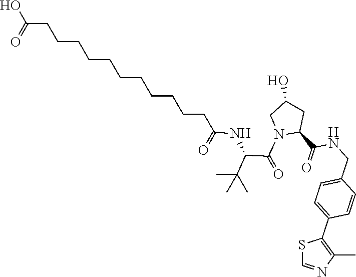 [SMILES: Cc1ncsc1-c1ccc(CNC(=O)[C@@H]2C[C@@H](O)CN2C(=O)[C@@H](NC(=O)CCCCCCCCCCCC(=O)O)C(C)(C)C)cc1] | I-52 | 13-[[(1S)-1-[(2S,4R)-4- hydroxy-2-[[4-(4-methylthiazol- 5-yl)phenyl]methyl carbamoyl]pyrrolidine-1- carbonyl]-2,2-dimethyl- propyl]amino]-13-oxo- tridecanoic acid | 657.3 |
| 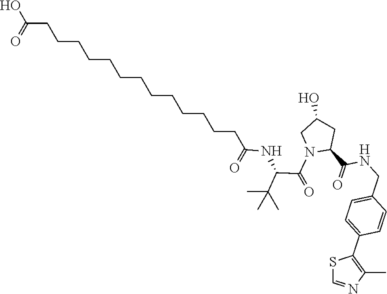 [SMILES: Cc1ncsc1-c1ccc(CNC(=O)[C@@H]2C[C@@H](O)CN2C(=O)[C@@H](NC(=O)CCCCCCCCCCCCCC(=O)O)C(C)(C)C)cc1] | I-53 | 15-(((S)-1-((2S,4R)-4-hydroxy- 2-((4-(4-methylthiazol-5- yl)benzyl)carbamoyl)pyrrolidin- 1-yl)-3,3-dimethyl-1-oxobutan- 2-yl)amino)-15- oxopentadecanoic acid | 685.4 |
| 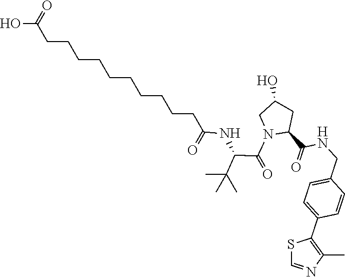 [SMILES: Cc1ncsc1-c1ccc(CNC(=O)[C@@H]2C[C@@H](O)CN2C(=O)[C@@H](NC(=O)CCCCCCCCCCC(=O)O)C(C)(C)C)cc1] | I-54 | 12-(((S)-1-((2S,4R)-4-hydroxy- 2-((4-(4-methylthiazol-5- yl)benzyl)carbamoyl)pyrrolidin- 1-yl)-3,3-dimethyl-1-oxobutan- 2-yl)amino)-12-oxododecanoic acid | 643.5 |
| 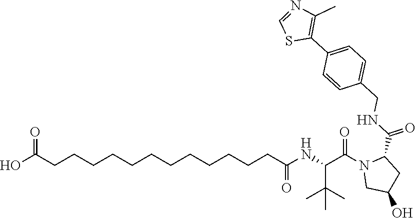 [SMILES: Cc1ncsc1-c1ccc(CNC(=O)[C@@H]2C[C@@H](O)CN2C(=O)[C@@H](NC(=O)CCCCCCCCCCCCC(=O)O)C(C)(C)C)cc1] | I-55 | 14-(((S)-1-((2S,4R)-4-hydroxy- 2-((4-(4-methylthiazol-5- yl)benzyl)carbamoyl)pyrrolidin- 1-yl)-3,3-dimethyl-1-oxobutan- 2-yl)amino)-14- oxotetradecanoic acid | 671.4 |
| 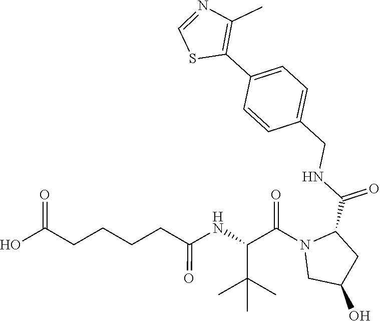 [SMILES: Cc1ncsc1-c1ccc(CNC(=O)[C@@H]2C[C@@H](O)CN2C(=O)[C@@H](NC(=O)CCCCC(=O)O)C(C)(C)C)cc1] | I-56 | 6-(((S)-1-((2S,4R)-4-hydroxy-2- ((4-(4-methylthiazol-5- yl)benzyl)carbamoyl)pyrrolidin- 1-yl)-3,3-dimethyl-1-oxobutan- 2-yl)amino)-6-oxohexanoic acid | 559.3 |

### Preparation of (S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecan-1-oic acid (I-57)

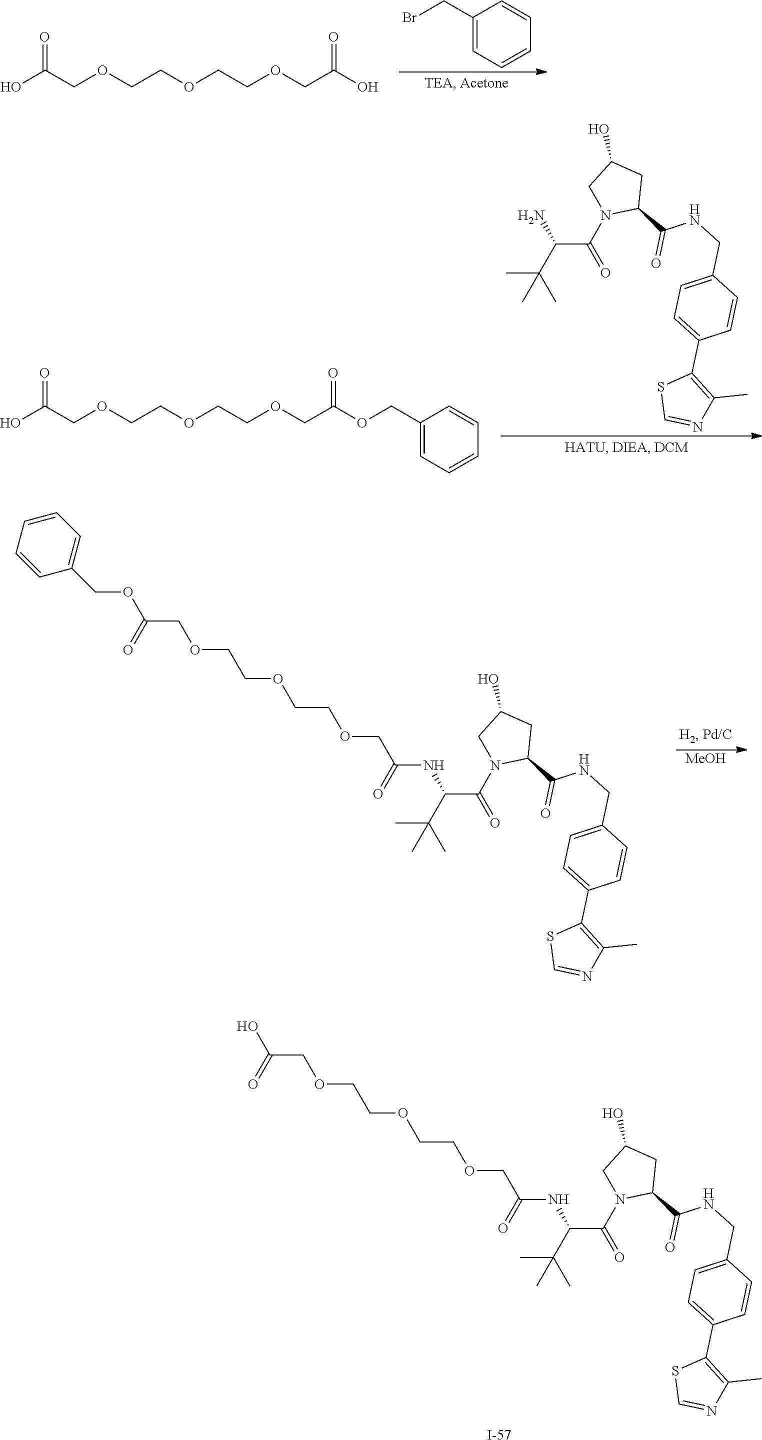

### Step 1: Preparation of 2-[2-[2-(2-benzyloxy-2-oxo-ethoxy)ethoxy]ethoxy]acetic acid

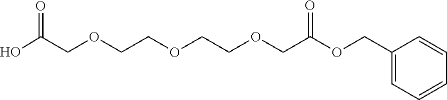 [SMILES: O=C(O)COCCOCCOCC(=O)OCc1ccccc1]

To a mixture of 2-[2-[2-(carboxymethoxy)ethoxy]ethoxy]acetic acid (3 g, 13.50 mmol) and TEA (3.51 g, 34.71 mmol, 4.83 mL) in acetone (20 mL) was added bromomethylbenzene (2.42 g, 14.18 mmol, 1.68 mL) dropwise at 0° C. The mixture was stirred at 20° C. for 16 h. A thick precipitate formed, was filtered off, and the filter cake was washed with acetone (10 mL). The filtrate was concentrated and the residue was dissolved in water (300 mL). The mixture was extracted by EtOAc (50 mL×3), then treated with 2 N HCl to pH 3-5. The mixture was extracted with EtOAc (50 mL×3) and the organic layers were combined and washed with brine (50 mL), then dried over Na₂SO₄, filtered and concentrated under vacuum to give 2-[2-[2-(2-benzyloxy-2-oxo-ethoxy)ethoxy]ethoxy]acetic acid (1.4 g, 33.20%) as a yellow oil which was used to next step without further purification. ¹H NMR (400 MHz, CDCl₃) δ=7.31-7.26 (m, 5H), 5.12 (s, 2H), 4.12 (s, 2H), 4.08 (s, 2H), 3.69-3.61 (m, 8H) ppm.

### Step 2: Preparation of (S)-benzyl 13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecan-1-oate

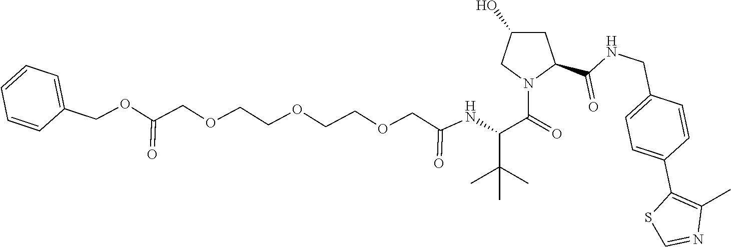 [SMILES: Cc1ncsc1-c1ccc(CNC(=O)[C@@H]2C[C@@H](O)CN2C(=O)[C@@H](NC(=O)COCCOCCOCC(=O)OCc2ccccc2)C(C)(C)C)cc1]

To a solution of 2-[2-[2-(2-benzyloxy-2-oxo-ethoxy)ethoxy]ethoxy]acetic acid (836.17 mg, 2.68 mmol) in DCM (10 mL) was added HATU (1.32 g, 3.48 mmol) and DIEA (900.50 mg, 6.97 mmol, 1.21 mL). (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (1 g, 2.14 mmol) was added. The mixture was stirred at 30° C. for 2 h. The mixture was concentrated under vacuum to a give yellow solid which was purified by reverse phase flash (ACN/0.1% formic acid in water) to give (S)-benzyl 13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecan-1-oate (1.2 g, 1.66 mmol, 71.28%) as a yellow oil. LCMS (ESI) m/z: [M+H]⁺=725.4. ¹H NMR (400 MHz, chloroform-d) δ=8.69 (s, 1H), 7.42-7.32 (m, 11H), 4.77-4.75 (m, 1H), 4.63-4.47 (m, 3H), 4.37-4.32 (m, 1H), 4.24-4.17 (m, 3H), 4.13 (d, J=11.6 Hz, 1H), 4.09-3.94 (m, 2H), 3.77-3.68 (m, 9H), 3.63-3.59 (m, 1H), 2.64-2.56 (m, 1H), 2.54 (s, 3H), 2.19-2.08 (m, 1H), 1.00-0.92 (m, 9H) ppm.

### Step 3: Preparation of(S)-13-((2S,4R)-4-hydroxy-2-((4-(4-methylthiazol-5-yl)benzyl)carbamoyl) pyrrolidine-1-carbonyl)-14,14-dimethyl-11-oxo-3,6,9-trioxa-12-azapentadecan-1-oic acid (I-57)

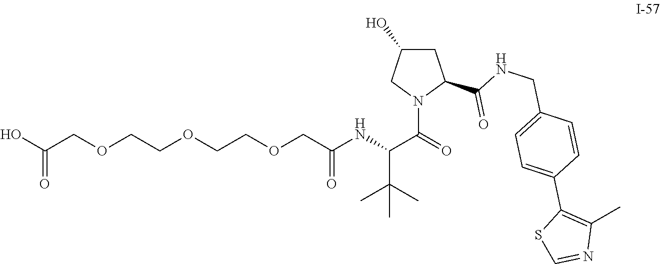 [SMILES: Cc1ncsc1-c1ccc(CNC(=O)[C@@H]2C[C@@H](O)CN2C(=O)[C@@H](NC(=O)COCCOCCOCC(=O)O)C(C)(C)C)cc1]

To a mixture of (S)-benzyl 13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecan-1-oate (1.1 g, 1.52 mmol) in MeOH (20 mL) was added 10% Pd/C (500 mg). The mixture was stirred at 25° C. for 12 h under 15 psi of hydrogen. The mixture was then stirred at 40° C. for 8 h. The mixture was filtered to remove Pd/C and the filtrate was concentrated under vacuum. The residue was purified by reversed phase chromatography to give I-57 (560 mg, 58.14%) as a white solid. LCMS (ESI) m/z: [M+H]⁺=635.2. ¹H NMR (400 MHz, methanol-d4) δ=8.94-8.86 (m, 1H), 7.50-7.43 (m, 4H), 4.62-4.50 (m, 3H), 4.43-4.34 (m, 1H), 4.12 (s, 2H), 4.08 (d, J=5.2 Hz, 2H), 3.93-3.86 (m, 1H), 3.85-3.79 (m, 1H), 3.76-3.70 (m, 8H), 2.51-2.49 (m, 3H), 2.29-2.21 (m, 1H), 2.16-2.07 (m, 1H), 1.06 (s, 9H) ppm. Chiral SFC: AD-3-MeOH+ACN (DEA)-40-3ML-35T.lcm, Rt=0.419 min, ee>100%.

The following intermediates in Table A10 were prepared in a similar manner as described in the preparation of I-57.

TABLE A10

| | | | LCMS |
|---|---|---|---|
| | Intermediate | | (ESI)m/z: |
| Structure | No. | Name | [M + H]⁺ |
| 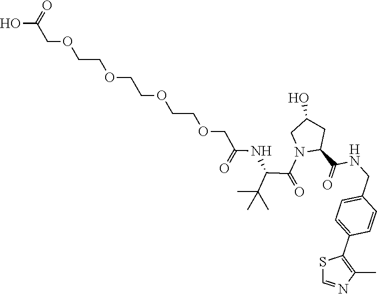 [SMILES: Cc1ncsc1-c1ccc(CNC(=O)[C@@H]2C[C@@H](O)CN2C(=O)[C@@H](NC(=O)COCCOCCOCCOCC(=O)O)C(C)(C)C)cc1] | 1-58 | (S)-16-((2S,4R)-4-hydroxy-2-((4- (4-methylthiazol-5- yl)benzyl)carbamoyl)pyrrolidine-1- carbonyl)-17,17-dimethyl-14-oxo- 3,6,9,12-tetraoxa-15- azaoctadecan-1-oic acid | 679.2 |

### Preparation of 2-(2,6-dioxopiperidin-3-yl)-5-(piperazin-1-yl)isoindole-1,3-dione; formic acid (I-59) and 5-[4-(2,2-diethoxyethyl)piperazin-1-yl]-2-(2,6-dioxopiperidin-3-yl)isoindole-1,3-dione (I-60)

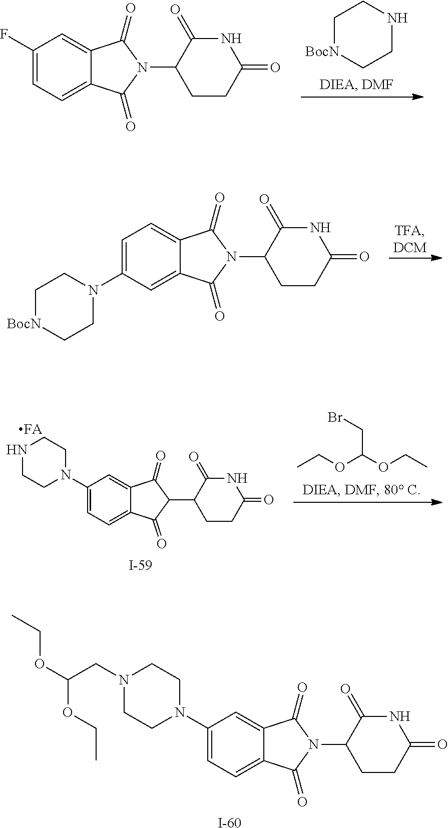

### Step 1: Preparation of tert-butyl 4-[2-(2,6-dioxopiperidin-3-yl)-1,3-dioxoisoindol-5-yl]piperazine-1-carboxylate

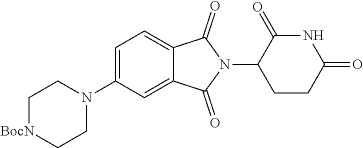 [SMILES: CC(C)(C)OC(=O)N1CCN(c2ccc3c(c2)C(=O)N(C2CCC(=O)NC2=O)C3=O)CC1]

To a solution of 2-(2,6-dioxopiperidin-3-yl)-5-fluoroisoindole-1,3-dione (5.40 g, 19.549 mmol, 1.00 equiv) and tert-butyl piperazine-1-carboxylate (3.64 g, 19.549 mmol, 1 equiv) in DMF (35.00 mL) was added DIEA (7.58 g, 58.648 mmol, 3 equiv) at room temperature. The resulting mixture was stirred for 5 h at 90° C. The reaction was quenched by the addition of water (50 mL) at room temperature. The resulting mixture was extracted with EtOAc (3×50 mL). The combined organic layers were washed with brine (3×100 mL), and dried over anhydrous Na₂SO₄. After filtration, the filtrate was concentrated under reduced pressure. This resulted in tert-butyl 4-[2-(2,6-dioxopiperidin-3-yl)-1,3-dioxoisoindol-5-yl]piperazine-1-carboxylate (7.32 g, 84.62%) as a light yellow solid. LCMS (ESI) m/z: [M+H]⁺=443.

### Step 2: Preparation of 2-(2,6-dioxopiperidin-3-yl)-5-(piperazin-1-yl)isoindole-1,3-dione formic acid (I-59)

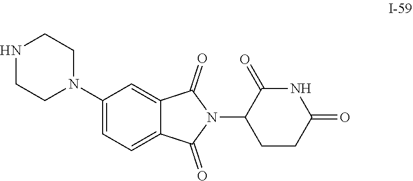 [SMILES: O=C1CCC(N2C(=O)c3ccc(N4CCNCC4)cc3C2=O)C(=O)N1]

To a solution of tert-butyl 4-[2-(2,6-dioxopiperidin-3-yl)-1,3-dioxoisoindol-5-yl]piperazine-1-carboxylate (900.00 mg, 2.034 mmol, 1.00 equiv) in DCM was added TFA (5.00 mL, 67.315 mmol, 33.09 equiv). The resulting mixture was stirred for 3 h at room temperature. The residue was purified by reversed phase flash chromatography with the following conditions: column, C18 silica gel; mobile phase, 0.1% formic acid in water/ACN, 0% to 100% gradient over 45 min; detector, UV 254 nm. This resulted in I-59 (786.8 mg, 99.41%) as a yellow solid. LCMS (ESI) m/z: [M+H]⁺=343.

### Step 3: Preparation of 5-[4-(2,2-diethoxyethyl)piperazin-1-yl]-2-(2,6-dioxopiperidin-3-yl)isoindole-1,3-dione (I-60)

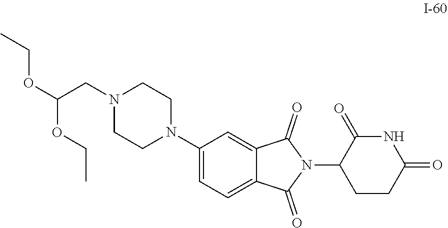 [SMILES: CCOC(CN1CCN(c2ccc3c(c2)C(=O)N(C2CCC(=O)NC2=O)C3=O)CC1)OCC]

To a solution of I-59 (1.15 g, 3.359 mmol, 1.00 equiv) and 2-bromo-1,1-diethoxyethane (0.66 g, 3.359 mmol, 1 equiv) in DMF (20.00 mL) was added DIEA (1.30 g, 10.077 mmol, 3 equiv). The resulting mixture was stirred for 12 h at 80° C. The residue was purified by reversed phase flash chromatography with the following conditions: column, C18 silica gel; mobile phase, ACN 0.1% formic acid in water, 0% to 100% gradient in 45 min; detector, UV 254 nm. This resulted in I-60 (987 mg, 64.08%) as a white solid; ¹H NMR (300 MHz, DMSO) δ 11.09 (s, 1H), 7.68 (d, 1H), 7.34 (d, 1H), 7.26 (dd, 1H), 5.08 (dd, 1H), 4.64 (t, 1H), 3.67-3.58 (m, 2H), 3.57-3.38 (m, 6H), 3.33 (s, 1H), 2.98-2.80 (m, 1H), 2.66-2.59 (m, 4H), 2.56 (s, 2H), 2.48 (s, 1H), 2.09-1.96 (m, 1H), 1.13 (t, 6H). LCMS (ESI) m/z: [M+H]⁺=459.30.

### Preparation of 1-(2-[[2-(2,6-dioxopiperidin-3-yl)-1,3-dioxoisoindol-5-yl]oxy]ethyl)piperidine-4-carboxylic acid (I-61)

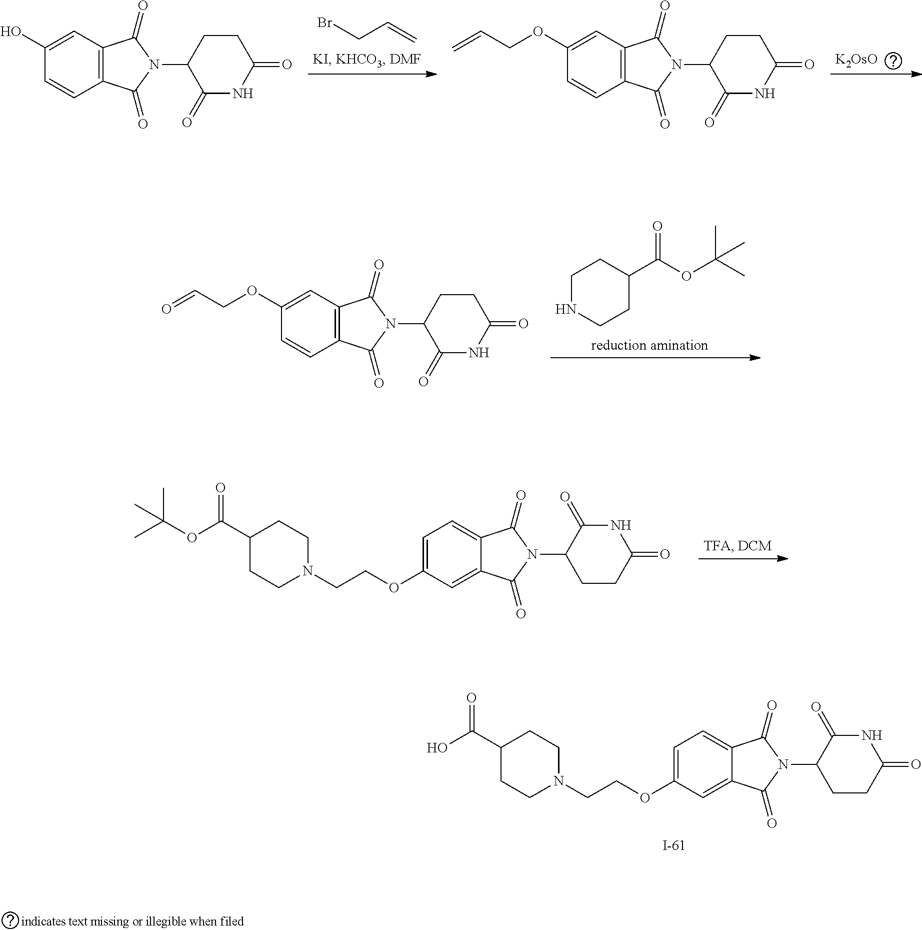

### Step 1: Preparation of 2-(2,6-dioxopiperidin-3-yl)-5-(prop-2-en-1-yloxy)isoindole-1,3-dione

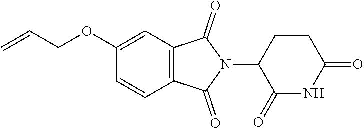 [SMILES: C=CCOc1ccc2c(c1)C(=O)N(C1CCC(=O)NC1=O)C2=O]

To a solution of 2-(2,6-dioxopiperidin-3-yl)-5-hydroxyisoindole-1,3-dione (5.48 g, 19.983 mmol, 1.00 equiv) and allyl bromide (3.63 g, 29.975 mmol, 1.5 equiv) in DMF (50.00 mL) were added KI (331.72 mg, 1.998 mmol, 0.1 equiv) and KHCO₃ (3.00 g, 29.975 mmol, 1.5 equiv). The resulting mixture was stirred for 12 h at 65° C., then diluted with water (100 mL) and extracted with EtOAc (3×50 mL). The combined organic layers were washed with brine (3×60 mL), and dried over anhydrous Na2SO4. After filtration, the filtrate was concentrated under reduced pressure. The residue was purified by silica gel column chromatography, eluted with hexane/EtOAc (1:1) to afford 2-(2,6-dioxopiperidin-3-yl)-5-(prop-2-en-1-yloxy)isoindole-1,3-dione (6.7 g, crude) as a yellow-green solid. LCMS (ESI) m/z: [M+H]⁺=315.

### Step 2: Preparation of 2-[[2-(2,6-dioxopiperidin-3-yl)-1,3-dioxoisoindol-5-yl]oxy]acetaldehyde

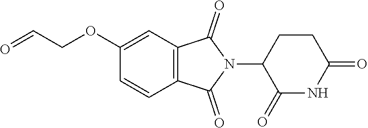 [SMILES: O=CCOc1ccc2c(c1)C(=O)N(C1CCC(=O)NC1=O)C2=O]

To a solution of 2-(2,6-dioxopiperidin-3-yl)-5-(prop-2-en-1-yloxy)isoindole-1,3-dione (3.14 g, 9.991 mmol, 1.00 equiv) in dioxane (30.00 mL) were added NaIO₄ (10.68 g, 49.953 mmol, 5.00 equiv), water (3.00 mL), and 2,6-lutidine (3.21 g, 29.972 mmol, 3 equiv). To the above mixture was added K₂OsO₄ dihydrate (0.37 g, 0.999 mmol, 0.1 equiv) at room temperature. The resulting mixture was stirred for additional an 2 h at room temperature. The reaction was quenched with water at room temperature and the resulting mixture was extracted with EtOAc (3×50 mL). The combined organic layers were washed with brine (3×100 mL), and dried over anhydrous Na₂SO₄. After filtration, the filtrate was concentrated under reduced pressure. This resulted in 2-[[2-(2,6-dioxopiperidin-3-yl)-1,3-dioxoisoindol-5-yl]oxy]acetaldehyde (1.83 g, 57.92%) as a light brown solid. LCMS (ESI) m/z: [M+H]⁺=317.

### Step 3: Preparation of tert-butyl 1-(2-[[2-(2,6-dioxopiperidin-3-yl)-1,3-dioxoisoindol-5-yl]oxy]ethyl)piperidine-4-carboxylate

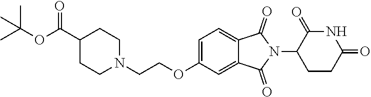 [SMILES: CC(C)(C)OC(=O)C1CCN(CCOc2ccc3c(c2)C(=O)N(C2CCC(=O)NC2=O)C3=O)CC1]

To a solution of 2-[[2-(2,6-dioxopiperidin-3-yl)-1,3-dioxoisoindol-5-yl]oxy]acetaldehyde (1.83 g, 5.786 mmol, 1.00 equiv) and tert-butyl piperidine-4-carboxylate (1.07 g, 5.786 mmol, 1.00 equiv) in DMF (35.00 mL) was added NaBH(OAc)₃ (3.68 g, 17.359 mmol, 3.00 equiv). The resulting mixture was stirred for 3 h at room temperature. The residue was purified by reversed phase flash chromatography with the following conditions: column, C18 silica gel; mobile phase, ACN/0.1% formic acid in water, 0% to 100% gradient in 45 min; detector, UV 254 nm. This resulted in tert-butyl 1-(2-[[2-(2,6-dioxopiperidin-3-yl)-1,3-dioxoisoindol-5-yl]oxy]ethyl)piperidine-4-carboxylate (1.16 g, 41.29%) as an off-white solid. LCMS (ESI) m/z: [M+H]⁺=401.

### Step 4: Preparation of 1-(2-[[2-(2,6-dioxopiperidin-3-yl)-1,3-dioxoisoindol-5-yl]oxy]ethyl)piperidine-4-carboxylic acid (I-61)

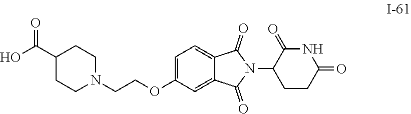 [SMILES: O=C1CCC(N2C(=O)c3ccc(OCCN4CCC(C(=O)O)CC4)cc3C2=O)C(=O)N1]

To a solution of tert-butyl 1-(2-[[2-(2,6-dioxopiperidin-3-yl)-1,3-dioxoisoindol-5-yl]oxy]ethyl)piperidine-4-carboxylate (1.16 g, 2.389 mmol, 1.00 equiv) in DCM (10.00 mL) was added TFA (10.00 mL, 134.630 mmol, 34.18 equiv). The resulting mixture was stirred for 5 h at room temperature. The residue was purified by reversed phase flash chromatography with the following conditions: column, C18 silica gel; mobile phase, CAN/0.1% formic acid in water, 0% to 100% gradient in 45 min; detector, UV 254 nm. This resulted in I-61 (845 mg, 73.42%) as a white solid. ¹H NMR (300 MHz, DMSO) δ 11.11 (s, 1H), 8.15 (d, 1H), 7.84 (d, 1H), 7.47 (d, 1H), 7.37 (dd, 1H), 5.12 (dd, 1H), 4.31 (t, 2H), 3.02-2.85 (m, 3H), 2.79 (t, 2H), 2.66-2.60 (m, 1H), 2.59-2.54 (m, 1H), 2.29-2.12 (m, 3H), 2.15-1.99 (m, 1H), 1.87-1.75 (m, 2H), 1.66-1.47 (m, 2H). LCMS (ESI) m/z: [M+H]⁺=430.15.

### Preparation of 4-(3-aminopropoxy)-2-(2,6-dioxopiperidin-3-yl)isoindoline-1,3-dione (K-32)

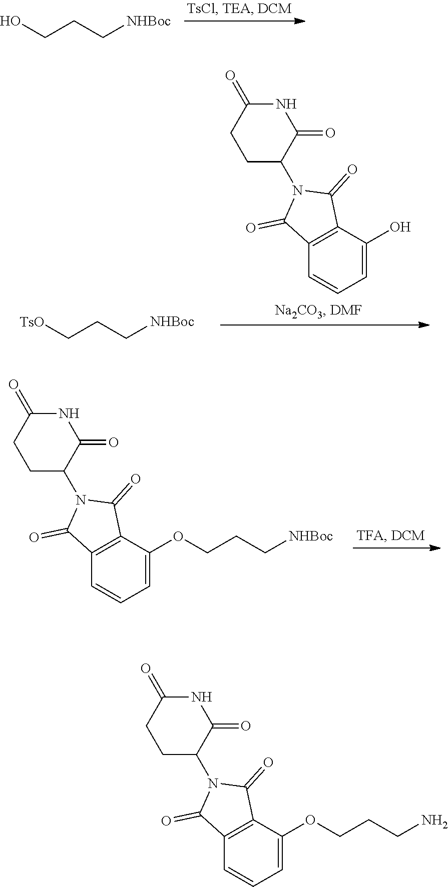 [SMILES: CC(C)(C)OC(=O)NCCCO ; O=C1CCC(N2C(=O)c3cccc(O)c3C2=O)C(=O)N1 ; Cc1ccc(S(=O)(=O)OCCCNC(=O)OC(C)(C)C)cc1 ; CC(C)(C)OC(=O)NCCCOc1cccc2c1C(=O)N(C1CCC(=O)NC1=O)C2=O ; NCCCOc1cccc2c1C(=O)N(C1CCC(=O)NC1=O)C2=O]

### Step 1: Preparation of tert-butyl N-[3-[(4-methylbenzenesulfonyl)oxy]propyl]carbamate

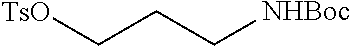 [SMILES: Cc1ccc(S(=O)(=O)OCCCNC(=O)OC(C)(C)C)cc1]

To a solution of tert-butyl N-(3-hydroxypropyl)carbamate (1.00 g, 5.707 mmol, 1.00 equiv) in DCM (40.00 mL) was added TsCl (1.32 g, 6.924 mmol, 1.21 equiv) and TEA (2.00 mL). The reaction mixture was stirred at room temperature overnight. The resulting mixture was neutralized to pH 8 with aqueous NaHCO₃. The organic layers were isolated and washed with brine (20 mL), then dried over anhydrous Na₂SO₄. After filtration, the filtrate was concentrated under reduced pressure. The residue was purified by silica gel column chromatography, eluting with PE/EtOAc (5:1), to afford tert-butyl N-[3-[(4-methylbenzenesulfonyl)oxy]propyl]carbamate (750 mg, 39.90%) as a colorless oil. LCMS (ESI) m/z: [M+H]⁺=330.

### Step 2: Preparation of tert-butyl N-(3-[[2-(2,6-dioxopiperidin-3-yl)-1,3-dioxoisoindol-4-yl]oxy]propyl)carbamate

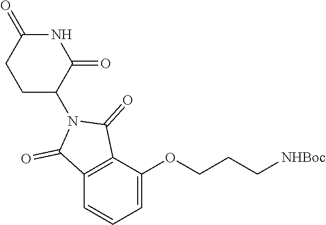 [SMILES: CC(C)(C)OC(=O)NCCCOc1cccc2c1C(=O)N(C1CCC(=O)NC1=O)C2=O]

A suspension of tert-butyl N-[3-[(4-methylbenzenesulfonyl)oxy]propyl]carbamate (225 mg), 2-(2,6-dioxopiperidin-3-yl)-4-hydroxyisoindole-1,3-dione (300 mg, 1.00 equiv) and Na₂CO₃ (200 mg) in DMF (2 mL) was prepared. The mixture was stirred at 80° C. for 2 h. The resulting mixture was diluted with DCM (10 mL) and washed with H₂O (2×10 mL). The organic layer was separated and dried over Na₂SO₄. After filtration, the filtrate was concentrated under reduced pressure. The residue was purified by Prep-TLC (eluting with PE/EtOAc 1:1) to afford tert-butyl N-(3-[[2-(2,6-dioxopiperidin-3-yl)-1,3-dioxoisoindol-4-yl]oxy]propyl)carbamate (70 mg, 14.83%) as a yellow solid. LCMS (ESI) m/z: [M+H]⁺=432.

### Step 3: Preparation of 4-(3-aminopropoxy)-2-(2,6-dioxopiperidin-3-yl)isoindole-1,3-dione (K-32)

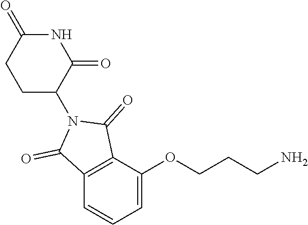 [SMILES: NCCCOc1cccc2c1C(=O)N(C1CCC(=O)NC1=O)C2=O]

To a solution of tert-butyl N-(3-[[2-(2,6-dioxopiperidin-3-yl)-1,3-dioxoisoindol-4-yl]oxy]propyl)carbamate (70.00 mg, 0.162 mmol, 1.00 equiv) in DCM (5.00 mL) was added TFA (1.00 mL). The mixture was stirred at room temperature for 0.5 h. The resulting mixture was concentrated under reduced pressure. The crude product 4-(3-aminopropoxy)-2-(2,6-dioxopiperidin-3-yl)isoindole-1,3-dione (70 mg) was used in the next step directly without further purification. LCMS (ESI) m/z: [M+H]⁺=332.

The following intermediates in Table A11 were prepared in a similar manner as described in the preparation of K-32.

TABLE A11

| | | | LCMS |
|---|---|---|---|
| | Intermediate | | (ESI)m/z: |
| Structure | No. | Name | [M + H]⁺ |
| 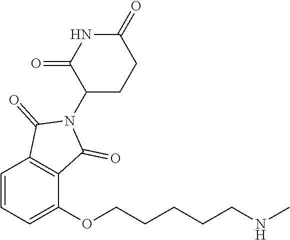 [SMILES: CNCCCCCOc1cccc2c1C(=O)N(C1CCC(=O)NC1=O)C2=O] | K-33 | 2-(2,6-dioxopiperidin-3-yl)-4-[[5- (methylamino)pentyl]oxy]isoindole- 1,3-dione | 374 |
| 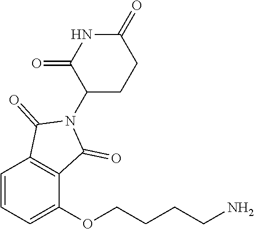 [SMILES: NCCCCOc1cccc2c1C(=O)N(C1CCC(=O)NC1=O)C2=O] | K-34 | 4-(4-aminobutoxy)-2-(2,6- dioxopiperidin-3-yl) isoindoline-1,3- dione | 346 |

### Preparation of 4-(2-aminoethoxy)-2-(2,6-dioxopiperidin-3-yl)isoindole-1,3-dione (K-35)

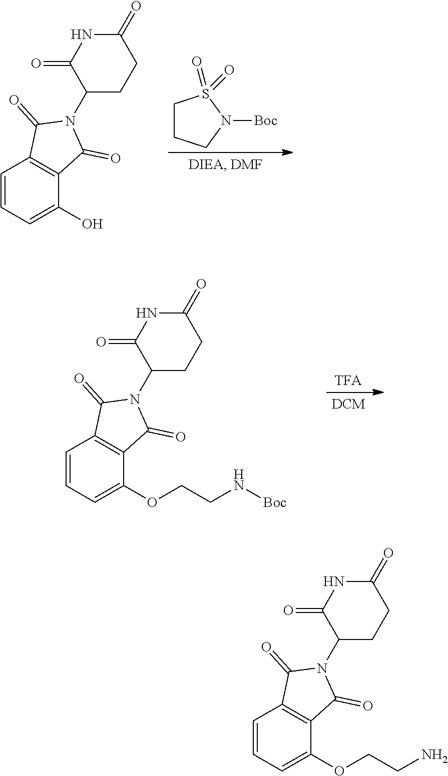 [SMILES: O=C1CCC(N2C(=O)c3cccc(O)c3C2=O)C(=O)N1 ; CC(C)(C)OC(=O)N1CCCS1(=O)=O ; CC(C)(C)OC(=O)NCCOc1cccc2c1C(=O)N(C1CCC(=O)NC1=O)C2=O ; NCCOc1cccc2c1C(=O)N(C1CCC(=O)NC1=O)C2=O]

### Step 1: Preparation of tert-butyl N-(2-[[2-(2,6-dioxopiperidin-3-yl)-1,3-dioxoisoindol-4-yl]oxy]ethyl)carbamate

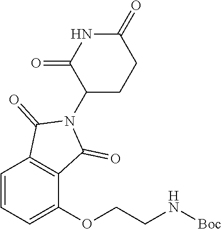 [SMILES: CC(C)(C)OC(=O)NCCOc1cccc2c1C(=O)N(C1CCC(=O)NC1=O)C2=O]

To a stirred mixture of 2-(2,6-dioxopiperidin-3-yl)-4-hydroxyisoindole-1,3-dione (500.00 mg, 1.823 mmol, 1.00 equiv) and tert-butyl 1,2,3-oxathiazolidine-3-carboxylate 2,2-dioxide (407.03 mg, 1.823 mmol, 1.00 equiv) in DMF (5.00 mL) was added DIEA (471.29 mg, 3.647 mmol, 2.00 equiv) dropwise at room temperature under nitrogen atmosphere. The resulting mixture was stirred for overnight at 80 degrees C. under nitrogen atmosphere. The resulting mixture was diluted with water (10 mL), then extracted with EtOAc (3×10 mL). The organic layers were combined and washed with brine (10 mL) and dried over anhydrous Na₂SO₄. After filtration, the filtrate was concentrated under reduced pressure. The residue was purified by reverse flash chromatography to afford tert-butyl N-(2-[[2-(2,6-dioxopiperidin-3-yl)-1,3-dioxoisoindol-4-yl]oxy]ethyl)carbamate (400 mg, 52.56%) as off-white solid. LCMS (ESI) m/z [M+H]⁺=418.

### Step 2: Preparation of 4-(2-aminoethoxy)-2-(2,6-dioxopiperidin-3-yl)isoindole-1,3-dione (K-35)

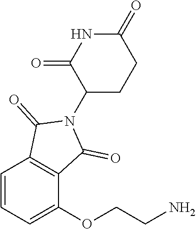 [SMILES: NCCOc1cccc2c1C(=O)N(C1CCC(=O)NC1=O)C2=O]

To a stirred solution of tert-butyl N-(2-[[2-(2,6-dioxopiperidin-3-yl)-1,3-dioxoisoindol-4-yl]oxy]ethyl)carbamate (150.00 mg, 0.359 mmol, 1.00 equiv) in DCM (4.00 mL) was added TFA (0.8 mL) dropwise at room temperature under nitrogen atmosphere. The resulting mixture was stirred for 1 h at room temperature under nitrogen atmosphere, then concentrated under reduced pressure to afford intermediate 3 (100 mg) as a colorless oil. The crude product 4-(2-aminoethoxy)-2-(2,6-dioxopiperidin-3-yl)isoindole-1,3-dione was used in the next step directly without further purification. LCMS (ESI) m/z [M+H]⁺=318.

### Preparation of 3-[1-[2-(2,6-dioxopiperidin-3-yl)-1,3-dioxoisoindol-4-yl]azetidin-3-yl]propanal (K-36)

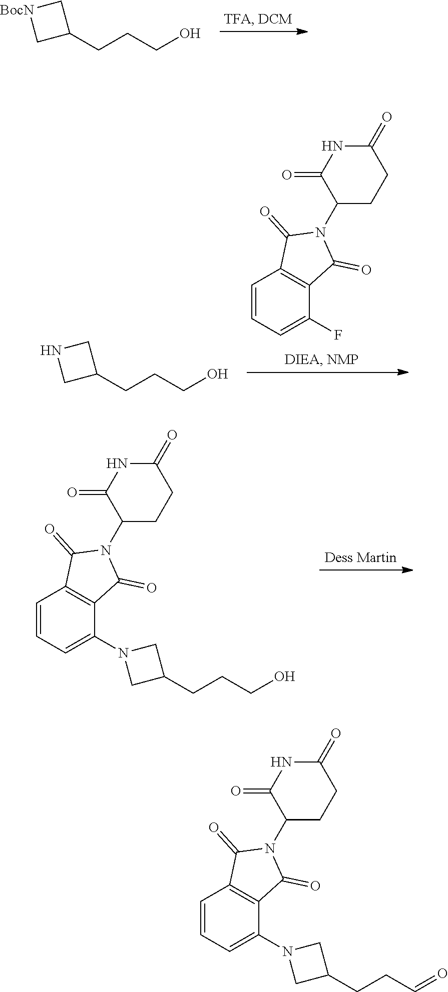

### Step 1: Preparation of 3-(azetidin-3-yl)propan-1-ol

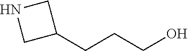

To a solution of tert-butyl 3-(3-hydroxypropyl)azetidine-1-carboxylate (120.00 mg, 0.557 mmol, 1.00 equiv) in DCM (2.00 mL) was added TFA (0.50 mL, 6.732 mmol, 12.08 equiv). The mixture was stirred at room temperature for 0.5 h. The reaction mixture was concentrated to afford 3-(azetidin-3-yl)propan-1-ol (70 mg, crude) as a yellow oil. LCMS (ESI) m/z [M+H]⁺=116.

### Step 2: Preparation of 2-(2,6-dioxopiperidin-3-yl)-4-[3-(3-hydroxypropyl)azetidin-1-yl]isoindole-1,3-dione

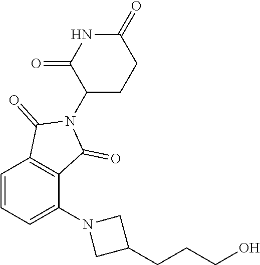 [SMILES: O=C1CCC(N2C(=O)c3cccc(N4CC(CCCO)C4)c3C2=O)C(=O)N1]

To a solution of 3-(azetidin-3-yl)propan-1-ol (70 mg, 1.337 mmol, 1.00 equiv) and 2-(2,6-dioxopiperidin-3-yl)-4-fluoroisoindole-1,3-dione (155.12 mg, 0.562 mmol, 1.00 equiv) in NMP (1 mL) was added DIEA (1 mL). The reaction mixture was stirred at 80 degrees C. for 2 h. The mixture was diluted with DCM (10 mL) and washed with brine (2×10 mL). The organic layer was dried over Na₂SO₄ and filtered. The filtrate was concentrated. The residue was purified by Prep-TLC (eluting with PE/EtOAc, 1:1) to afford 2-(2,6-dioxopiperidin-3-yl)-4-[3-(3-hydroxypropyl)azetidin-1-yl]isoindole-1,3-dione (100 mg, 47.95%) as a yellow solid. LCMS (ESI) m/z: [M+H]⁺=372.

### Step 3: Preparation of 3-[1-[2-(2,6-dioxopiperidin-3-yl)-1,3-dioxoisoindol-4-yl]azetidin-3-yl]propanal (Intermediate K-36)

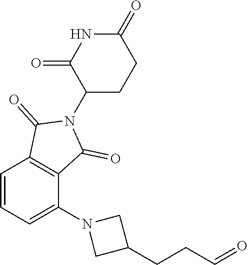 [SMILES: O=CCCC1CN(c2cccc3c2C(=O)N(C2CCC(=O)NC2=O)C3=O)C1]

2-(2,6-dioxopiperidin-3-yl)-4-[3-(3-hydroxypropyl)azetidin-1-yl]isoindole-1,3-dione (100.00 mg, 0.269 mmol, 1.00 equiv) and Dess-Martin periodinane (114.20 mg, 0.269 mmol, 1.00 equiv) were suspended in DCM (4.00 mL). The mixture was stirred overnight at room temperature. The mixture was purified by Prep-TLC (eluting with 1:1 PE/EtOAc) to afford 3-[1-[2-(2,6-dioxopiperidin-3-yl)-1,3-dioxoisoindol-4-yl]azetidin-3-yl]propanal (60 mg, 60.33%) as a yellow solid. LCMS (ESI) m/z [M+H]⁺=370.

The following intermediates in Table A12 were prepared in a similar manner as described in the preparation of K-36.

TABLE A12

| | | | LCMS |
|---|---|---|---|
| | Intermediate | | (ESI)m/z: |
| Structure | No. | Name | [M + H]⁺ |
| 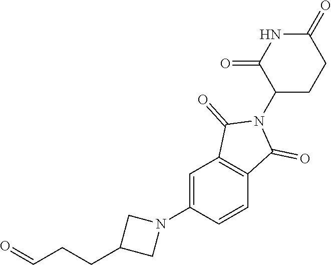 [SMILES: O=CCCC1CN(c2ccc3c(c2)C(=O)N(C2CCC(=O)NC2=O)C3=O)C1] | K-37 | 3-[1-[2-(2,6-dioxopiperidin-3-yl)- 1,3-dioxoisoindol-5-yl]azetidin-3- yl]propanal | 370 |
| 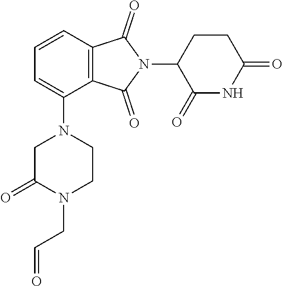 [SMILES: O=CCN1CCN(c2cccc3c2C(=O)N(C2CCC(=O)NC2=O)C3=O)CC1=O] | K-38 | 2-(4-(2-(2,6-dioxopiperidin-3-yl)- 1,3-dioxoisoindolin-4-yl)-2- oxopiperazin-1-yl)acetaldehyde | 399 |

### Preparation of 5-((2-(2,6-dioxopiperidin-3-yl)-1,3-dioxoisoindolin-4-yl)oxy)pentanal (K-39)

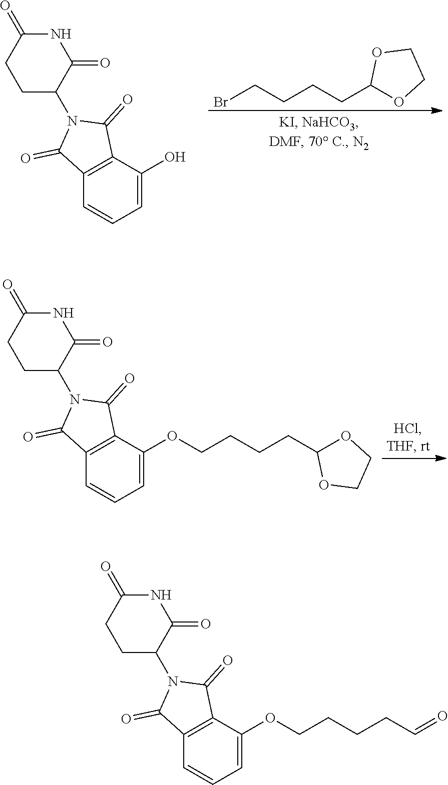 [SMILES: O=C1CCC(N2C(=O)c3cccc(O)c3C2=O)C(=O)N1 ; BrCCCCC1OCCO1 ; O=C1CCC(N2C(=O)c3cccc(OCCCCC4OCCO4)c3C2=O)C(=O)N1 ; O=CCCCCOc1cccc2c1C(=O)N(C1CCC(=O)NC1=O)C2=O]

### Step 1: Preparation of 4-(4-(1,3-dioxolan-2-yl)butoxy)-2-(2,6-dioxopiperidin-3-yl)isoindoline-1,3-dione

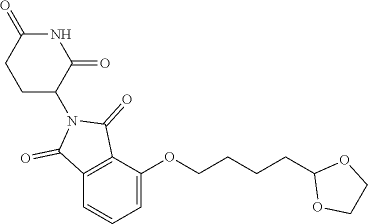 [SMILES: O=C1CCC(N2C(=O)c3cccc(OCCCCC4OCCO4)c3C2=O)C(=O)N1]

A solution of 2-(2,6-dioxopiperidin-3-yl)-4-hydroxyisoindole-1,3-dione (500.00 mg, 1.823 mmol, 1.00 equiv.), 2-(4-bromobutyl)-1,3-dioxolane (457.46 mg, 2.188 mmol, 1.20 equiv.), KI (60.53 mg, 0.365 mmol, 0.20 equiv.) and NaHCO₃ (306.33 mg, 3.646 mmol, 2.00 equiv.) in DMF (5.00 mL) was stirred for 24 h at 70 degrees C. under nitrogen atmosphere. The resulting mixture was diluted with ethyl acetate (20 mL), then washed with saturated NaCl (3×20 mL). The organic layers were dried over anhydrous Na₂SO₄, then filtered. The filtrate was concentrated under reduced pressure and the residue was purified by silica gel column chromatography, eluting with DCM/MeOH=100/1 to afford 4-(4-(1,3-dioxolan-2-yl)butoxy)-2-(2,6-dioxopiperidin-3-yl)isoindoline-1,3-dione (600 mg, 81.78%) as a light yellow solid. LCMS (ESI) m/z [M+H]⁺=403.

### Step 2: Preparation of 5-((2-(2,6-dioxopiperidin-3-yl)-1,3-dioxoisoindolin-4-yl)oxy)pentanal (K-39)

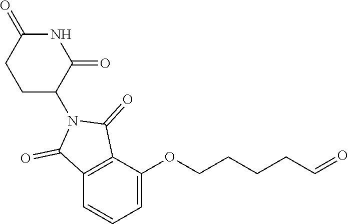 [SMILES: O=CCCCCOc1cccc2c1C(=O)N(C1CCC(=O)NC1=O)C2=O]

A solution of 4-(4-(1,3-dioxolan-2-yl)butoxy)-2-(2,6-dioxopiperidin-3-yl)isoindoline-1,3-dione (170.00 mg, 0.422 mmol, 1.00 equiv.) in HCl (4.00 mL)/THF (4.00 mL) was stirred for 10 h at room temperature. The mixture was neutralized to pH 7 with saturated NaHCO₃. The resulting mixture was extracted with ethyl acetate (3×20 mL). The combined organic layers were washed with saturated brine (20 mL), then dried over anhydrous Na₂SO₄. After filtration, the filtrate was concentrated under reduced pressure to afford crude 5-((2-(2,6-dioxopiperidin-3-yl)-1,3-dioxoisoindolin-4-yl)oxy)pentanal (140 mg, 92.48%) which was used in the next step directly without further purification. LCMS (ESI) m/z [M+H]⁺=359.

### Preparation of 2-(4-(3-amino-6-(2-hydroxyphenyl)pyridazin-4-yl)piperazin-1-yl)acetic acid (I-62)

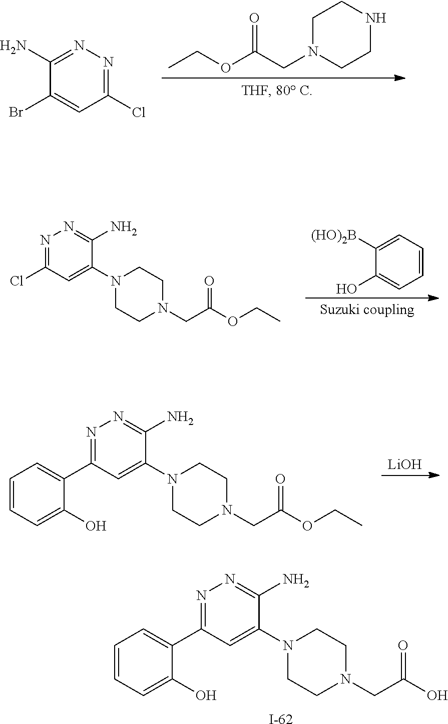

### Step 1: Preparation of ethyl 2-[4-(3-amino-6-chloropyridazin-4-yl)piperazin-1-yl]acetate

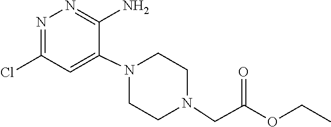 [SMILES: CCOC(=O)CN1CCN(c2cc(Cl)nnc2N)CC1]

To a stirred mixture of 4-bromo-6-chloropyridazin-3-amine (420.00 mg, 2.015 mmol, 1.00 equiv) and ethyl 2-(piperazin-1-yl)acetate (381.74 mg, 2.216 mmol, 1.10 equiv) in DMF (3.00 mL) was added DIEA (1.30 g, 10.059 mmol, 4.99 equiv) in portions at 120° C. over 12 h under an atmosphere of dry nitrogen. The mixture was allowed to cool down to room temperature. The residue was purified by reversed phase flash chromatography to afford ethyl 2-[4-(3-amino-6-chloropyridazin-4-yl)piperazin-1-yl]acetate (340 mg, 50.66%) as a light yellow solid. LCMS (ESI) m/z: [M+H]⁺=299.76.

### Step 2: Preparation of ethyl 2-[4-[3-amino-6-(2-hydroxyphenyl)pyridazin-4-yl]piperazin-1-yl]acetate

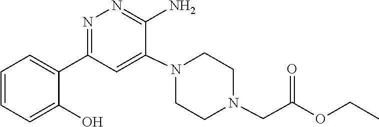 [SMILES: CCOC(=O)CN1CCN(c2cc(-c3ccccc3O)nnc2N)CC1]

To a stirred solution of ethyl 2-[4-(3-amino-6-chloropyridazin-4-yl)piperazin-1-yl]acetate (340.00 mg, 1.134 mmol, 1.00 equiv) and 2-hydroxyphenylboronic acid (234.67 mg, 1.701 mmol, 1.50 equiv) in dioxane:water (5 mL, 4:1) was added potassium carbonate (391.90 mg, 2.836 mmol, 2.50 equiv) and XPhos Pd G₃ (192.02 mg, 0.227 mmol, 0.20 equiv) in portions at 100° C. over 2 h under an atmosphere of dry nitrogen. The residue was purified by reversed phase flash chromatography to deliver ethyl 2-[4-[3-amino-6-(2-hydroxyphenyl)pyridazin-4-yl]piperazin-1-yl]acetate (253 mg, 58.04%) as an off-white solid. LCMS (ESI) m/z: [M+H]⁺=357.41.

### Step 3: Preparation 2-(4-(3-amino-6-(2-hydroxyphenyl)pyridazin-4-yl)piperazin-1-yl)acetic acid (I-62)

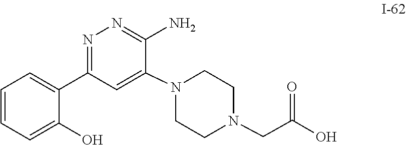 [SMILES: Nc1nnc(-c2ccccc2O)cc1N1CCN(CC(=O)O)CC1]

To a solution of ethyl 2-[4-[3-amino-6-(2-hydroxyphenyl)pyridazin-4-yl]piperazin-1-yl]acetate (250.00 mg, 0.699 mmol, 1.00 equiv) was added portionwise a solution of LiOH (348.92 mg, 14.570 mmol, 20.83 equiv) in 1:1 THF/water (10 mL) at room temperature over 2 h. The residue was purified by reversed phase flash chromatography to afford 2-(4-(3-amino-6-(2-hydroxyphenyl)pyridazin-4-yl)piperazin-1-yl)acetic acid (I-62, 192 mg, 76.67%) as an off-white solid. LCMS (ESI) m/z: [M+H]+=329.36.

### Preparation of N-[2-[(2-aminoethyl)(methyl)amino]ethyl]-2-[[2-(2,6-dioxopiperidin-3-yl)-1,3-dioxo-2,3-dihydro-1H-isoindol-4-yl]oxy]acetamide hydrochloride (I-63)

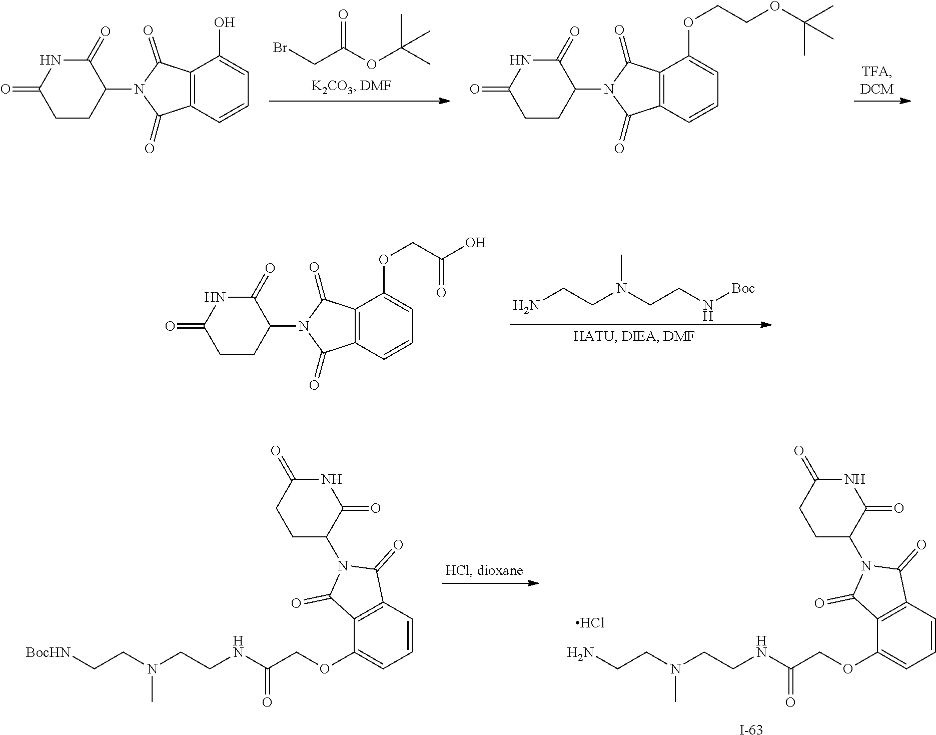

### Step 1: Preparation of tert-butyl 2-[[2-(2,6-dioxopiperidin-3-yl)-1,3-dioxoisoindol-4-yl]oxy]acetate

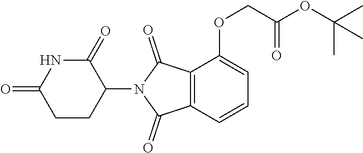 [SMILES: CC(C)(C)OC(=O)COc1cccc2c1C(=O)N(C1CCC(=O)NC1=O)C2=O]

To a solution of 2-(2,6-dioxopiperidin-3-yl)-4-hydroxyisoindole-1,3-dione (5.50 g, 20.056 mmol, 1.00 equiv) in DMF (65.0 mL) were added tert-butyl 2-bromoacetate (3.91 g, 20.056 mmol, 1 equiv) and K₂CO₃ (8.32 g, 60.168 mmol, 3 equiv). The resulting solution was stirred at room temperature for 12 h. The solution was concentrated under reduced pressure. The residue was purified by silica gel column chromatography, eluting with petroleum ether/EtOAc (1:1) to afford tert-butyl 2-[[2-(2,6-dioxopiperidin-3-yl)-1,3-dioxoisoindol-4-yl]oxy]acetate (5.2 g, 66.76%) as a white solid. LCMS (ESI) m/z: [M+H]⁺=389.

### Step 2: Preparation of [[2-(2,6-dioxopiperidin-3-yl)-1,3-dioxoisoindol-4-yl]oxy]acetic acid

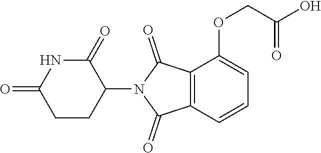 [SMILES: O=C(O)COc1cccc2c1C(=O)N(C1CCC(=O)NC1=O)C2=O]

A solution of tert-butyl 2-[[2-(2,6-dioxopiperidin-3-yl)-1,3-dioxoisoindol-4-yl]oxy]acetate (5.32 g, 13.698 mmol, 1.00 equiv) in 4 N HCl in dioxane (50.0 mL, 1645.594 mmol, 120.13 equiv) was stirred for 12 h at room temperature. The solution was concentrated under reduced pressure. The residue was purified by reversed phase flash chromatography (C18 silica gel column), eluting with 0-100% ACN in water over 35 minutes, detected at UV 254 nm. This resulted in [[2-(2,6-dioxopiperidin-3-yl)-1,3-dioxoisoindol-4-yl]oxy]acetic acid (4.66 g, 100%) as a white solid. LCMS (ESI) m/z: [M+H]⁺=333.

### Step 3: Preparation of tert-butyl N-(2-[[2-(2-[[2-(2,6-dioxopiperidin-3-yl)-1,3-dioxoisoindol-4-yl]oxy]acetamido)ethyl](methyl)amino]ethyl)carbamate

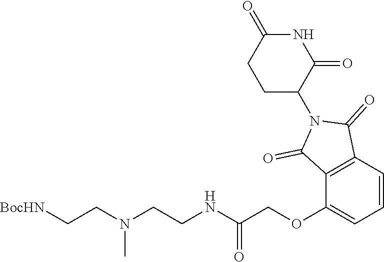 [SMILES: CN(CCNC(=O)COc1cccc2c1C(=O)N(C1CCC(=O)NC1=O)C2=O)CCNC(=O)OC(C)(C)C]

To a solution of [[2-(2,6-dioxopiperidin-3-yl)-1,3-dioxoisoindol-4-yl]oxy]acetic acid (926.00 mg, 2.787 mmol, 1.00 equiv) in DCM (35.00 mL) were added tert-butyl N-[2-[(2-aminoethyl)(methyl)amino]ethyl]carbamate (908.45 mg, 4.180 mmol, 1.5 equiv), HATU (1.59 g, 4.180 mmol, 1.5 equiv) and DIEA (1.08 g, 8.361 mmol, 3 equiv). The resulting solution was stirred at room temperature for 3 h. The residue was purified by silica gel column chromatography, eluting with petroleum ether/EtOAc (1:1) to afford tert-butyl N-(2-[[2-(2-[[2-(2,6-dioxopiperidin-3-yl)-1,3-dioxoisoindol-4-yl]oxy]acetamido)ethyl](methyl)amino]ethyl)carbamate (1.527 g, crude) as a light yellow solid. LCMS (ESI) m/z: [M+H]⁺=532.

### Step 4: Preparation of N-[2-[(2-aminoethyl)(methyl)amino]ethyl]-2-[[2-(2,6-dioxopiperidin-3-yl)-1,3-dioxo-2,3-dihydro-1H-isoindol-4-yl]oxy]acetamide hydrochloride (I-63)

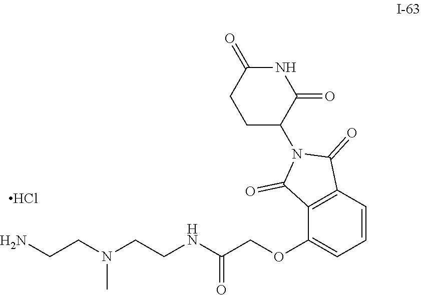 [SMILES: Cl ; CN(CCN)CCNC(=O)COc1cccc2c1C(=O)N(C1CCC(=O)NC1=O)C2=O]

A solution of tert-butyl N-(2-[[2-(2-[[2-(2,6-dioxopiperidin-3-yl)-1,3-dioxoisoindol-4-yl]oxy]acetamido)ethyl](methyl)amino]ethyl)carbamate (600.00 mg, 1.129 mmol, 1.00 equiv) was prepared in 4M HCl (123.47 mg, 3.386 mmol, 3.00 equiv) in dioxane (20.00 mL) stirred at room temperature for 2 h. The resulting mixture was concentrated under reduced pressure. The residue was purified by reversed phase flash chromatography (C18 silica gel column), eluting with 10-50% ACN in water over 10 minutes, detected at UV 254 nm, to afford N-[2-[(2-aminoethyl)(methyl)amino]ethyl]-2-[[2-(2,6-dioxopiperidin-3-yl)-1,3-dioxo-2,3-dihydro-1H-isoindol-4-yl]oxy]acetamide (I-63, 200 mg, 41.07%) as a yellow solid. LCMS (ESI) m/z: [M+H]⁺=432.18.

### Preparation of 2-(2,6-dioxopiperidin-3-yl)-4-[2-(piperazin-1-yl)ethoxy] isoindole-1,3-dione (K-40)

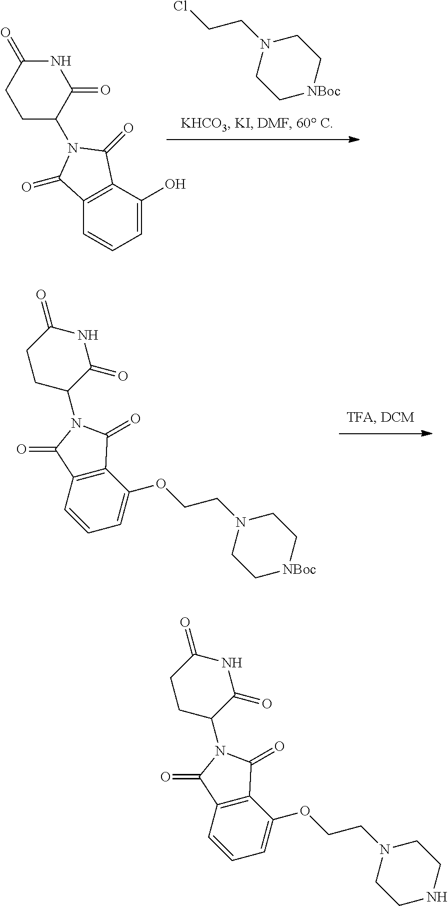 [SMILES: O=C1CCC(N2C(=O)c3cccc(O)c3C2=O)C(=O)N1 ; CC(C)(C)OC(=O)N1CCN(CCCl)CC1 ; CC(C)(C)OC(=O)N1CCN(CCOc2cccc3c2C(=O)N(C2CCC(=O)NC2=O)C3=O)CC1 ; O=C1CCC(N2C(=O)c3cccc(OCCN4CCNCC4)c3C2=O)C(=O)N1]

### Step 1: Preparation of tert-butyl 4-(2-[[2-(2,6-dioxopiperidin-3-yl)-1,3-dioxoisoindol-4-yl]oxy]ethyl)piperazine-1-carboxylate

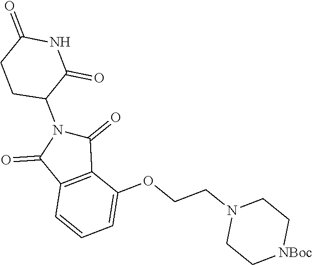 [SMILES: CC(C)(C)OC(=O)N1CCN(CCOc2cccc3c2C(=O)N(C2CCC(=O)NC2=O)C3=O)CC1]

To a stirred mixture of 2-(2,6-dioxopiperidin-3-yl)-4-hydroxyisoindole-1,3-dione (300.00 mg, 1.094 mmol, 1.00 equiv) and tert-butyl 4-(2-chloroethyl)piperazine-1-carboxylate (299.34 mg, 1.203 mmol, 1.10 equiv) in DMF (5.0 mL) were added KHCO₃ (219.04 mg, 2.188 mmol, 2.00 equiv) and KI (18.16 mg, 0.109 mmol, 0.10 equiv). The resulting mixture was stirred for 3 h at 60 degrees C. under a nitrogen atmosphere. The mixture was allowed to cool to room temperature, then filtered through a short pad of Celite and concentrated in vacuo. The residue was purified by reverse phase flash chromatography to afford tert-butyl 4-(2-[[2-(2,6-dioxopiperidin-3-yl)-1,3-dioxoisoindol-4-yl] oxy]ethyl)piperazine-1-carboxylate (200 mg, 35.70%) as a yellow solid. LCMS (ESI) m/z [M+H]⁺=487.

### Step 2: Preparation of 2-(2,6-dioxopiperidin-3-yl)-4-[2-(piperazin-1-yl)ethoxy] isoindole-1,3-dione (K-40)

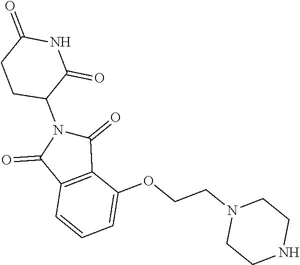 [SMILES: O=C1CCC(N2C(=O)c3cccc(OCCN4CCNCC4)c3C2=O)C(=O)N1]

A solution of tert-butyl 4-(2-[[2-(2,6-dioxopiperidin-3-yl)-1,3-dioxoisoindol-4-yl]oxy]ethyl)piperazine-1-carboxylate (200.00 mg, 0.413 mmol, 1.00 equiv) and TFA (1.0 mL) in DCM (3.0 mL) was stirred at 25 degrees C. for 1 h. The resulting mixture was concentrated under reduced pressure to afford crude 2-(2,6-dioxopiperidin-3-yl)-4-[2-(piperazin-1-yl)ethoxy] isoindole-1,3-dione (301 mg) as a white solid, which was used in the next step directly without further purification. LCMS (ESI) m/z [M+H]⁺=387.

### Preparation of 5-((2-(2,6-dioxopiperidin-3-yl)-3-oxoisoindolin-5-yl)oxy)pentanoic acid (K-41) and 5-((2-(2,6-dioxopiperidin-3-yl)-1-oxoisoindolin-5-yl)oxy)pentanoic acid (K-42)

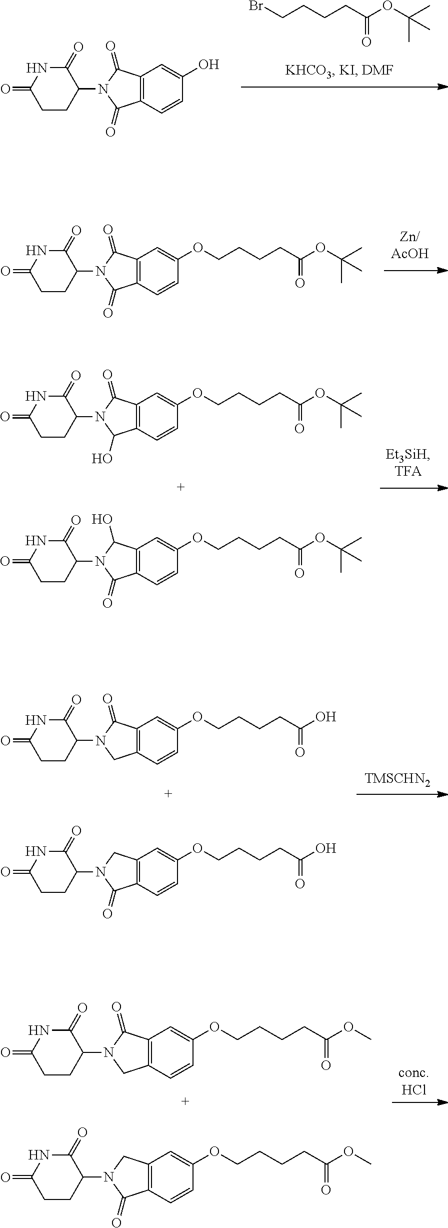

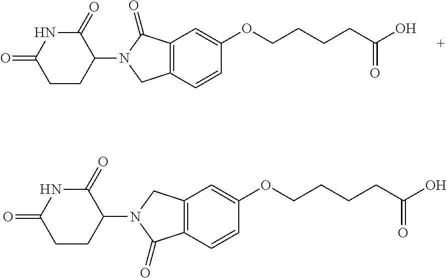 [SMILES: O=C(O)CCCCOc1ccc2c(c1)C(=O)N(C1CCC(=O)NC1=O)C2 ; O=C(O)CCCCOc1ccc2c(c1)CN(C1CCC(=O)NC1=O)C2=O]

### Step 1: Preparation of tert-butyl 5-((2-(2,6-dioxopiperidin-3-yl)-1,3-dioxoisoindolin-5-yl)oxy)pentanoate

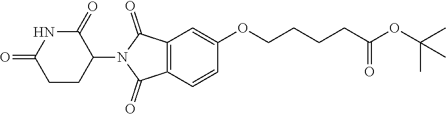 [SMILES: CC(C)(C)OC(=O)CCCCOc1ccc2c(c1)C(=O)N(C1CCC(=O)NC1=O)C2=O]

To a solution of 2-(2,6-dioxopiperidin-3-yl)-5-hydroxyisoindole-1,3-dione (1.00 g, 3.647 mmol, 1.00 equiv) and tert-butyl 5-bromopentanoate (0.95 g, 4.011 mmol, 1.10 equiv) in DMF (15.00 mL) was added KI (0.06 g, 0.365 mmol, 0.10 equiv) and KHCO₃ (0.73 g, 7.293 mmol, 2.00 equiv). The resulting mixture was stirred overnight at 60 degrees C. The resulting mixture was diluted with water (100 mL), extracted with EtOAc (100 mL×3), and the combined organic layers were washed with brine (50 mL), dried over anhydrous sodium sulfate, filtered, concentrated. The residue was purified by silica gel column chromatography, eluting with EtOAc in PE from 0% to 30% to afford tert-butyl 5-((2-(2,6-dioxopiperidin-3-yl)-1,3-dioxoisoindolin-5-yl)oxy)pentanoate (910.0 mg, 52.1%) as a light blue solid. LCMS (ESI) m/z: [M+H]⁺=431.

### Step 2: Preparation of tert-butyl 5-((2-(2,6-dioxopiperidin-3-yl)-1-hydroxy-3-oxoisoindolin-5-yl)oxy)pentanoate and tert-butyl 5-((2-(2,6-dioxopiperidin-3-yl)-3-hydroxy-1-oxoisoindolin-5-yl)oxy)pentanoate

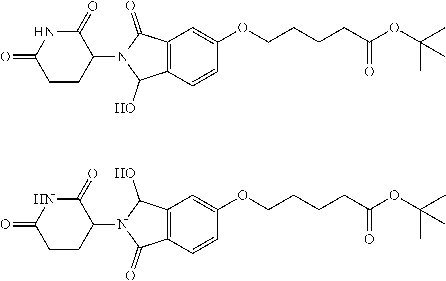 [SMILES: CC(C)(C)OC(=O)CCCCOc1ccc2c(c1)C(=O)N(C1CCC(=O)NC1=O)C2O ; CC(C)(C)OC(=O)CCCCOc1ccc2c(c1)C(O)N(C1CCC(=O)NC1=O)C2=O]

To a stirred mixture of tert-butyl 5-((2-(2,6-dioxopiperidin-3-yl)-1,3-dioxoisoindolin-5-yl)oxy)pentanoate (910.0 mg, 2.114 mmol, 1.00 equiv) in AcOH (30 mL) was added Zn (2.76 g, 42.281 mmol, 20.00 equiv). The resulting mixture was stirred for 2 hrs at 60 degrees C. The resulting mixture was filtered and the filtrate was concentrated under reduced pressure. The residue was purified by reverse phase flash chromatography with the following conditions: column, C18 silica gel; mobile phase, ACN in water, 10% to 50% gradient in 10 min; detector, UV 254 nm to afford a mixture of the title compounds (1.10 g, crude) as a light yellow solid. LCMS (ESI) m/z [M+H]⁺=433.

### Step 3: Preparation of 5-((2-(2,6-dioxopiperidin-3-yl)-3-oxoisoindolin-5-yl)oxy)pentanoic acid and 5-((2-(2,6-dioxopiperidin-3-yl)-1-oxoisoindolin-5-yl)oxy)pentanoic acid

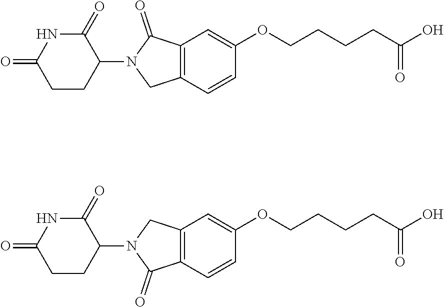 [SMILES: O=C(O)CCCCOc1ccc2c(c1)C(=O)N(C1CCC(=O)NC1=O)C2 ; O=C(O)CCCCOc1ccc2c(c1)CN(C1CCC(=O)NC1=O)C2=O]

To a stirred solution of tert-butyl 5-((2-(2,6-dioxopiperidin-3-yl)-1-hydroxy-3-oxoisoindolin-5-yl)oxy)pentanoate and tert-butyl 5-((2-(2,6-dioxopiperidin-3-yl)-3-hydroxy-1-oxoisoindolin-5-yl)oxy)pentanoate (1.10 g crude) in TFA (30 mL) was added Et₃SiH (6 mL). The resulting mixture was stirred for 2 hrs at room temperature. The reaction mixture was filtered through a short pad of Celite and concentrated in vacuo. The residue was purified by reverse flash phase chromatography with the following conditions: column, C18 silica gel; mobile phase, ACN in water, 10% to 50% gradient in 10 min; detector, UV 254 nm to afford the title compounds (820.0 mg, 89.5%). LCMS (ESI) m/z: [M+H]⁺=221.10.

### Step 4: Preparation of methyl 5-((2-(2,6-dioxopiperidin-3-yl)-3-oxoisoindolin-5-yl)oxy)pentanoate and methyl 5-((2-(2,6-dioxopiperidin-3-yl)-1-oxoisoindolin-5-yl)oxy)pentanoate

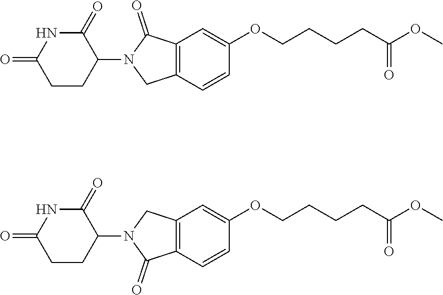 [SMILES: COC(=O)CCCCOc1ccc2c(c1)C(=O)N(C1CCC(=O)NC1=O)C2 ; COC(=O)CCCCOc1ccc2c(c1)CN(C1CCC(=O)NC1=O)C2=O]

To a stirred solution of 5-((2-(2,6-dioxopiperidin-3-yl)-3-oxoisoindolin-5-yl)oxy)pentanoic acid and 5-((2-(2,6-dioxopiperidin-3-yl)-1-oxoisoindolin-5-yl)oxy)pentanoic acid (820.0 mg, 2.277 mmol) in MeOH (30 mL) was added TMSCHN₂ (6 mL) at 0 degrees C. The resulting mixture was stirred for 2 hrs at room temperature. The reaction mixture was filtered through a short pad of Celite and concentrated in vacuo. The residue was purified by Prep-HPLC with follow conditions: Column: Xcelect CSH F-pheny OBD Column, 19*250 mm, 5 um; Mobile Phase A:Water (0.05% FA), Mobile Phase B:ACN; Flow rate: 25 mL/min; Gradient: 24% B to 40% B in 10 min to afford methyl 5-((2-(2,6-dioxopiperidin-3-yl)-3-oxoisoindolin-5-yl)oxy)pentanoate (80.0 mg, 10.1%) and methyl 5-((2-(2,6-dioxopiperidin-3-yl)-1-oxoisoindolin-5-yl)oxy)pentanoate (400.0 mg, 50.6%) as white solids. LCMS (ESI) m/z: [M+H]⁺=375.

### Step 5: Preparation of 5-((2-(2,6-dioxopiperidin-3-yl)-3-oxoisoindolin-5-yl)oxy)pentanoic acid (K-41) and 5-((2-(2,6-dioxopiperidin-3-yl)-1-oxoisoindolin-5-yl)oxy)pentanoic acid (K-42)

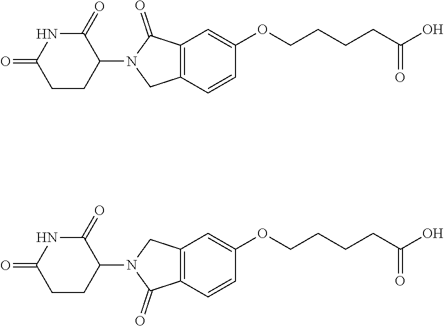 [SMILES: O=C(O)CCCCOc1ccc2c(c1)C(=O)N(C1CCC(=O)NC1=O)C2 ; O=C(O)CCCCOc1ccc2c(c1)CN(C1CCC(=O)NC1=O)C2=O]

To a stirred solution of methyl 5-((2-(2,6-dioxopiperidin-3-yl)-3-oxoisoindolin-5-yl)oxy)pentanoate (80.0 mg, 0.213 mmol, 1.00 equiv) in THF (1 mL) was added concentrated HCl (0.5 mL). The resulting mixture was stirred for an hour, the mixture was concentrated under vacuum to afford of 5-((2-(2,6-dioxopiperidin-3-yl)-3-oxoisoindolin-5-yl)oxy)pentanoic acid (90.0 mg, crude) as a white solid. LCMS (ESI) m/z: [M+H]⁺=361

To a stirred solution of methyl 5-((2-(2,6-dioxopiperidin-3-yl)-1-oxoisoindolin-5-yl)oxy)pentanoate (400.0 mg, 1.069 mmol, 1.00 equiv) in THF (3 mL) was added concentrated HCl (1.5 mL). The resulting mixture was stirred for an hour, the mixture was concentrated under vacuum to afford 5-((2-(2,6-dioxopiperidin-3-yl)-1-oxoisoindolin-5-yl)oxy)pentanoic acid (500.0 mg, crude) as a white solid. LCMS (ESI) m/z: [M+H]⁺=361.

### Preparation of 5-((2-(2,6-dioxopiperidin-3-yl)-1-oxoisoindolin-4-yl)oxy)pentanoic acid (K-43)

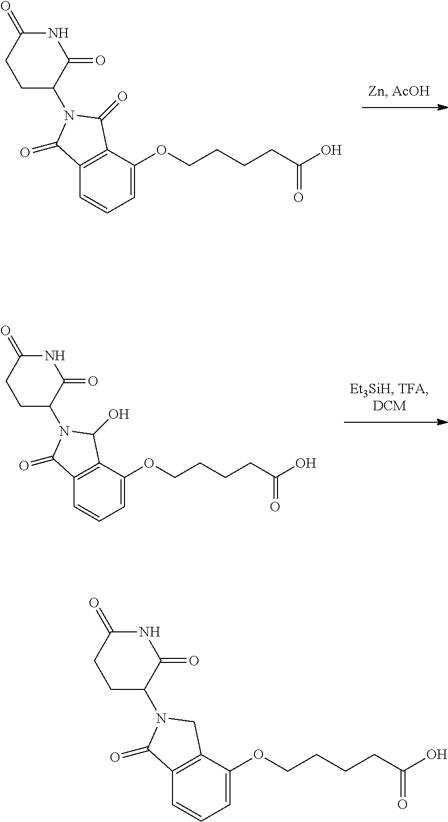 [SMILES: O=C(O)CCCCOc1cccc2c1C(=O)N(C1CCC(=O)NC1=O)C2=O ; O=C(O)CCCCOc1cccc2c1C(O)N(C1CCC(=O)NC1=O)C2=O ; O=C(O)CCCCOc1cccc2c1CN(C1CCC(=O)NC1=O)C2=O]

### Step 1: Preparation of 5-((2-(2,6-dioxopiperidin-3-yl)-3-hydroxy-1-oxoisoindolin-4-yl)oxy)pentanoic acid

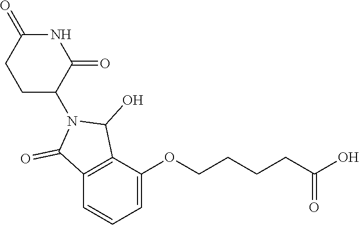 [SMILES: O=C(O)CCCCOc1cccc2c1C(O)N(C1CCC(=O)NC1=O)C2=O]

To a stirred solution of 5-[[2-(2,6-dioxopiperidin-3-yl)-1,3-dioxoisoindol-4-yl]oxy]pentanoic acid (I-30), (70.0 mg, 0.187 mmol, 1.00 equiv) in AcOH (3 mL) was added Zn (122.3 mg, 1.870 mmol, 10.00 equiv). The resulting mixture was stirred for 12 h at room temperature. The reaction mixture was filtered through a short pad of Celite and concentrated in vacuo. This resulted in crude 5-((2-(2,6-dioxopiperidin-3-yl)-3-hydroxy-1-oxoisoindolin-4-yl)oxy)pentanoic acid (117 mg, crude) as a light yellow solid. LCMS (ESI) m/z [M+H]⁺=377.

### Step 2: Preparation of 5-((2-(2,6-dioxopiperidin-3-yl)-1-oxoisoindolin-4-yl)oxy)pentanoic acid (K-43)

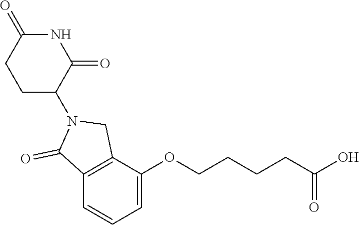 [SMILES: O=C(O)CCCCOc1cccc2c1CN(C1CCC(=O)NC1=O)C2=O]

To a stirred mixture of 5-((2-(2,6-dioxopiperidin-3-yl)-3-hydroxy-1-oxoisoindolin-4-yl)oxy)pentanoic acid (117.0 mg, 0.311 mmol, 1.00 equiv) and Et₃SiH (2 mL) in DCM (4 mL) was added TFA (1 mL) at room temperature. The resulting mixture was stirred for 12 h. The resulting mixture was concentrated under reduced pressure. The residue was purified by reverse phase flash chromatography with the following conditions: (Column, C18 silica gel; mobile phase, ACN in water, 0% to 80% gradient in 40 min; detector, UV 254 nm) to afford 5-((2-(2,6-dioxopiperidin-3-yl)-1-oxoisoindolin-4-yl)oxy)pentanoic acid (17.0 mg, 15.17%) as a white solid. LCMS (ESI) m/z [M+H]⁺=361.

### Preparation of 5-((2-(2,6-dioxopiperidin-3-yl)-1-oxoisoindolin-5-yl)amino)pentanoic acid (K-44)

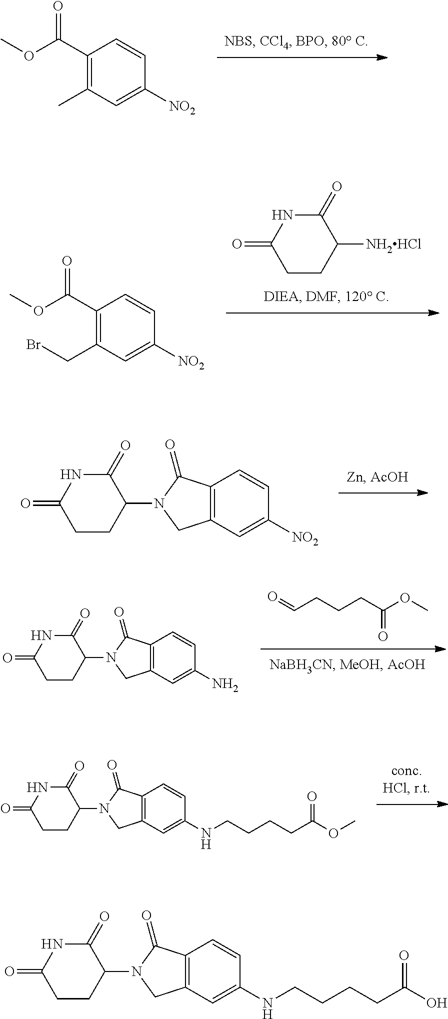

### Step 1: Preparation of methyl 2-(bromomethyl)-4-nitrobenzoate

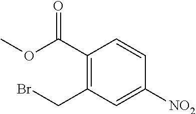

To a stirred solution of methyl 2-methyl-4-nitrobenzoate (2.00 g, 10.247 mmol, 1.00 equiv) in CCl₄ (20.00 mL) was added NBS (1.82 g, 0.010 mmol, 1.00 equiv) and BPO (0.21 g, 0.001 mmol, 0.08 equiv) at room temperature. The resulting mixture was stirred for 3 h at 80 degrees C. The mixture was allowed to cool down to room temperature. The reaction mixture was filtered through a short pad of Celite and concentrated in vacuo. The residue was purified by silica gel column chromatography, eluting with PE/EtOAc (10:1) to afford methyl 2-(bromomethyl)-4-nitrobenzoate (2.1 g, 74.77%) as a white solid. LCMS (ESI) m/z: [M+H]+=274.07.

### Step 2: Preparation of 3-(5-nitro-1-oxoisoindolin-2-yl)piperidine-2,6-dione

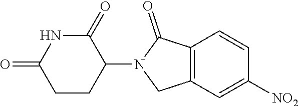 [SMILES: O=C1CCC(N2Cc3cc([N+](=O)[O-])ccc3C2=O)C(=O)N1]

To a stirred mixture of methyl 2-(bromomethyl)-4-nitrobenzoate (2.05 g, 7.480 mmol, 1.00 equiv) and the aminoglutarimide (1.02 g, 0.009 mmol, 1.20 equiv) in DMF (1.00 mL) was added DIEA (2.90 g, 0.022 mmol, 3.00 equiv) at room temperature. The resulting mixture was stirred for overnight at 120 degrees C. The mixture was allowed to cool to room temperature, then filtered through a short pad of Celite and concentrated in vacuo. The residue was purified by reverse flash chromatography with the following conditions: column, C18 silica gel; mobile phase, ACN in water, 0% to 100% gradient in 30 min; detector, UV 254 nm to afford 3-(5-nitro-1-oxoisoindolin-2-yl)piperidine-2,6-dione (940 mg, 34.76%) as a black solid. LCMS (ESI) m/z: [M+H]+=289.25.

### Step 3: Preparation of 3-(5-amino-1-oxoisoindolin-2-yl)piperidine-2,6-dione

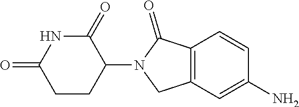 [SMILES: Nc1ccc2c(c1)CN(C1CCC(=O)NC1=O)C2=O]

To a stirred solution of 3-(5-nitro-1-oxoisoindolin-2-yl)piperidine-2,6-dione (380.00 mg, 1.314 mmol, 1.00 equiv) in AcOH (10.00 mL) was added Zn (859.31 mg, 13.138 mmol, 10 equiv) at room temperature. The resulting mixture was stirred for 4 h at room temperature. The resulting mixture was filtered and the filter cake was washed with H₂O (3×3 mL). The filtrate was concentrated under reduced pressure to afford 3-(5-amino-1-oxoisoindolin-2-yl)piperidine-2,6-dione (320 mg, 56.37%) as a black solid that was used in the next step directly without further purification. LCMS (ESI) m/z: [M+H]+=259.27.

### Step 4: Preparation of methyl 5-((2-(2,6-dioxopiperidin-3-yl)-1-oxoisoindolin-5-yl)amino)pentanoate

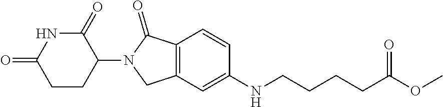 [SMILES: COC(=O)CCCCNc1ccc2c(c1)CN(C1CCC(=O)NC1=O)C2=O]

To a stirred mixture of 3-(5-amino-1-oxoisoindolin-2-yl)piperidine-2,6-dione (600.00 mg, 2.314 mmol, 1.00 equiv) and methyl 5-oxopentanoate (451.77 mg, 3.471 mmol, 1.50 equiv) in MeOH (20.00 mL) were added NaBH₃CN (436.29 mg, 6.943 mmol, 3.00 equiv) and AcOH (0.60 mL, 9.981 mmol, 4.52 equiv) at room temperature. The resulting mixture was stirred for 4 h at room temperature. The reaction mixture was filtered through a short pad of Celite and concentrated in vacuo. The residue was purified by reverse flash chromatography with the following conditions: column, C18 silica gel; mobile phase, ACN in water, 0% to 100% gradient in 30 min; detector, UV 254 nm to afford methyl 5-((2-(2,6-dioxopiperidin-3-yl)-1-oxoisoindolin-5-yl)amino)pentanoate (630 mg, 67.80%) as a grey solid. LCMS (ESI) m/z: [M+H]+=373.41.

### Step 5: Preparation of 5-((2-(2,6-dioxopiperidin-3-yl)-1-oxoisoindolin-5-yl)amino)pentanoic acid (K-44)

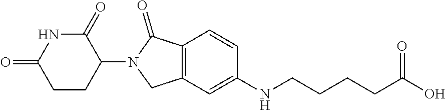 [SMILES: O=C(O)CCCCNc1ccc2c(c1)CN(C1CCC(=O)NC1=O)C2=O]

A solution of methyl 5-((2-(2,6-dioxopiperidin-3-yl)-1-oxoisoindolin-5-yl)amino)pentanoate (600 mg, 1.607 mmol, 1.00 equiv) in 2N HCl (15.00 mL, 30.000 mmol, 18.67 equiv) was stirred for overnight at room temperature. The reaction mixture was filtered through a short pad of Celite and concentrated in vacuo. The residue was purified by reverse flash chromatography with the following conditions: column, C18 silica gel; mobile phase, ACN in water, 0% to 100% gradient in 30 min; detector, UV 254 nm to afford the title compound (233 mg, 38.74%) as a grey solid. LCMS (ESI) m/z: [M+H]+=359.38.

### Preparation of 4-[3-(3-aminopropyl)azetidin-1-yl]-2-(2,6-dioxopiperidin-3-yl)isoindole-1,3-dione (K-45)

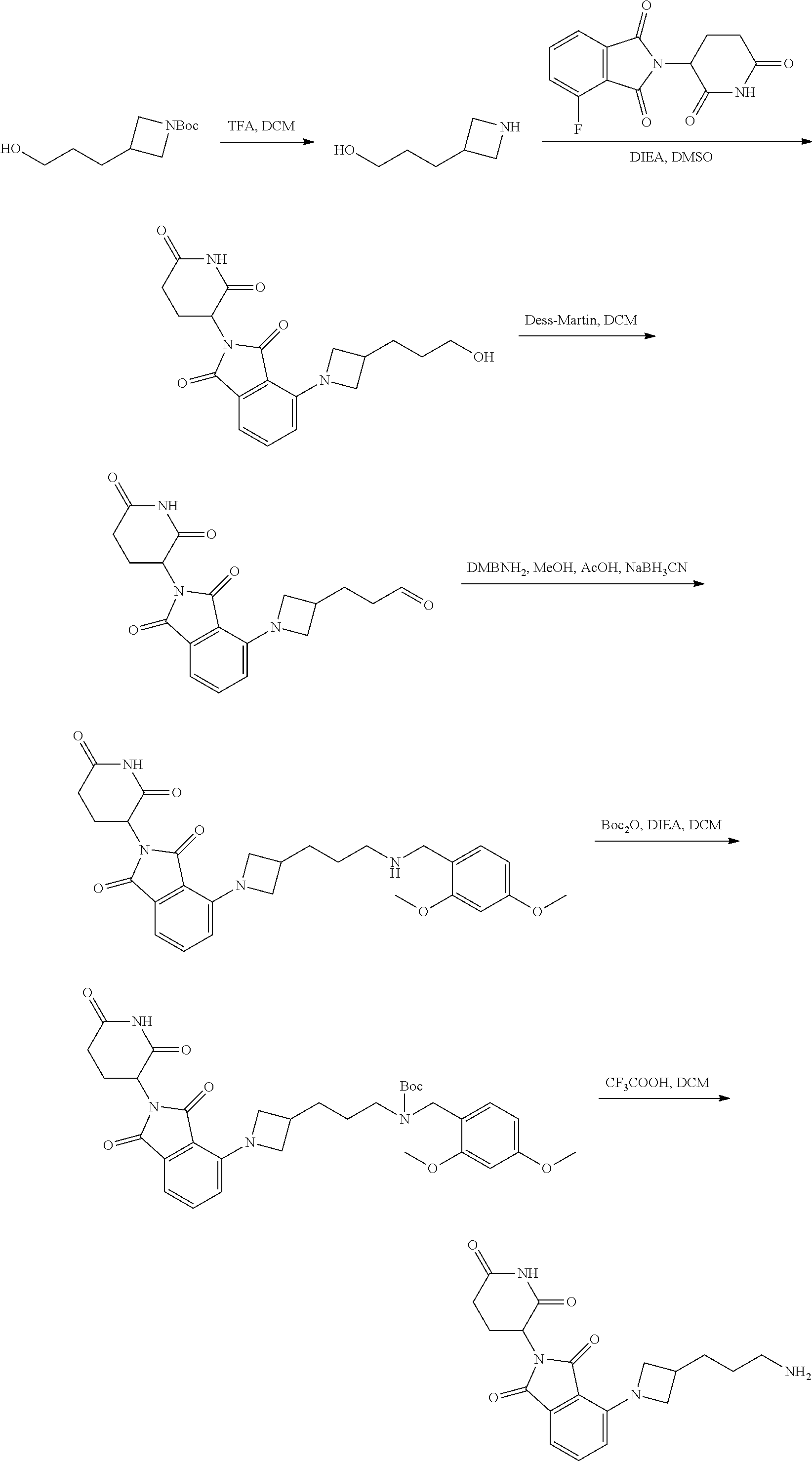

### Step 1: Preparation of 3-(azetidin-3-yl)propan-1-ol

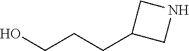

To a solution of tert-butyl 3-(3-hydroxypropyl)azetidine-1-carboxylate (400.00 mg, 1.858 mmol, 1.00 equiv) in DCM (4.00 mL) was added TFA (4.00 mL). The mixture was stirred at room temperature for 2 h. The reaction mixture was concentrated to afford 3-(azetidin-3-yl)propan-1-ol (200 mg, crude) as a yellow oil.

### Step 2: Preparation of 2-(2,6-dioxopiperidin-3-yl)-4-[3-(3-hydroxypropyl)azetidin-1-yl]isoindole-1,3-dione

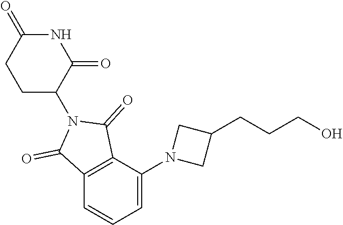 [SMILES: O=C1CCC(N2C(=O)c3cccc(N4CC(CCCO)C4)c3C2=O)C(=O)N1]

To a stirred mixture of 3-(azetidin-3-yl)propan-1-ol (200.00 mg, 1.736 mmol, 1.00 equiv) and 2-(2,6-dioxopiperidin-3-yl)-4-fluoroisoindole-1,3-dione (479.65 mg, 1.736 mmol, 1.00 equiv) in DMSO (1 mL) was added DIEA (2244.27 mg, 17.365 mmol, 10.00 equiv) at room temperature under a nitrogen atmosphere. The reaction mixture was stirred at 80 degrees C. for 16 h. The reaction solution was purified by reverse phase flash column chromatography with the following conditions: column C18 spherical gel; Mobile Phase A: Water, Mobile Phase B: MeOH; Flow rate: 40 mL/min; Gradient: 0% B to 100% B in 20 min; 254/220 nm. This resulted in of 2-(2,6-dioxopiperidin-3-yl)-4-[3-(3-hydroxypropyl)azetidin-1-yl]isoindole-1,3-dione (300 mg, 46.52%) as a yellow oil. LCMS (ESI) m/z [M+H]⁺=372.

### Step 3: Preparation of 3-[1-[2-(2,6-dioxopiperidin-3-yl)-1,3-dioxoisoindol-4-yl]azetidin-3-yl]propanal

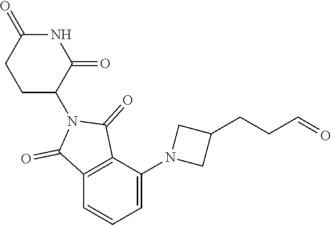 [SMILES: O=CCCC1CN(c2cccc3c2C(=O)N(C2CCC(=O)NC2=O)C3=O)C1]

To a stirred mixture of 2-(2,6-dioxopiperidin-3-yl)-4-[3-(3-hydroxypropyl)azetidin-1-yl]isoindole-1,3-dione (505.0 mg, 1.360 mmol, 1.00 equiv) in DCM (10.00 mL) was added Dess-Martin periodinane at room temperature under nitrogen atmosphere. The mixture was stirred overnight at room temperature. The mixture was purified by Prep-TLC (PE/EtOAc 1:1) to afford 3-[1-[2-(2,6-dioxopiperidin-3-yl)-1,3-dioxoisoindol-4-yl]azetidin-3-yl]propanal (322 mg, 64.2%) as a yellow solid. LCMS (ESI) m/z [M+H]⁺=370.

### Step 4: Preparation of 4-[3-(3-[[(2,4-dimethoxyphenyl)methyl]amino]propyl)azetidin-1-yl]-2-(2,6-dioxopiperidin-3-yl)isoindole-1,3-dione

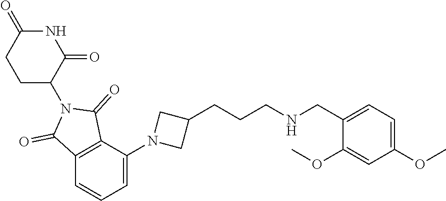 [SMILES: COc1ccc(CNCCCC2CN(c3cccc4c3C(=O)N(C3CCC(=O)NC3=O)C4=O)C2)c(OC)c1]

To a stirred solution of 3-[1-[2-(2,6-dioxopiperidin-3-yl)-1,3-dioxoisoindol-4-yl]azetidin-3-yl]propanal (200.00 mg, 0.541 mmol, 1.00 equiv) and 1-(2,4-dimethoxyphenyl)methanamine (181.07 mg, 1.082 mmol, 2.00 equiv) in MeOH (10 mL) was added AcOH (97.55 mg, 1.623 mmol, 3.00 equiv) and NaBH₃CN (102.08 mg, 1.623 mmol, 3.00 equiv) at room temperature under a nitrogen atmosphere. The resulting mixture was stirred for 16 h at room temperature. The reaction was quenched with water/ice at 0 degrees C. The resulting mixture was extracted with EtOAc (3×20 mL). The combined organic layers were washed with brine (2×20 mL), then dried over anhydrous Na₂SO₄. After filtration, the filtrate was concentrated under reduced pressure. The crude product was purified by flash chromatography under the following conditions: column C18 spherical gel; Mobile Phase A: Water, Mobile Phase B: ACN; Flow rate: 40 mL/min; Gradient: 0% B to 100% B in 20 min; 254/220 nm. This resulted in 4-[3-(3-[[(2,4-dimethoxyphenyl)methyl]amino]propyl)azetidin-1-yl]-2-(2,6-dioxopiperidin-3-yl)isoindole-1,3-dione (150 mg, 53.22%) as a yellow solid. LCMS (ESI) m/z [M+H]⁺=521.

### Step 5: Preparation of tert-butyl N-[(2,4-dimethoxyphenyl)methyl]-N-(3-[1-[2-(2,6-dioxopiperidin-3-yl)-1,3-dioxoisoindol-4-yl]azetidin-3-yl]propyl)carbamate

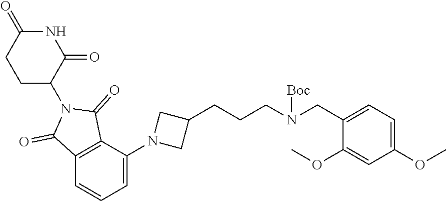 [SMILES: COc1ccc(CN(CCCC2CN(c3cccc4c3C(=O)N(C3CCC(=O)NC3=O)C4=O)C2)C(=O)OC(C)(C)C)c(OC)c1]

To a stirred solution of 4-[3-(3-[[(2,4-dimethoxyphenyl)methyl]amino]propyl)azetidin-1-yl]-2-(2,6-dioxopiperidin-3-yl)isoindole-1,3-dione (70.00 mg, 0.134 mmol, 1.00 equiv) and DIEA (52.14 mg, 0.403 mmol, 3.00 equiv) in DCM (4.00 mL) were added Boc₂O (58.69 mg, 0.269 mmol, 2.00 equiv) at 0 degrees C. under nitrogen atmosphere. The resulting mixture was stirred for 3 h at room temperature, then concentrated under vacuum. The crude product was purified by flash with the following conditions: column C18 spherical gel; Mobile Phase A: Water, Mobile Phase B: ACN; Flow rate: 40 mL/min; Gradient: 0% B to 100% B in 20 min; 254/220 nm. This resulted in tert-butyl N-[(2,4-dimethoxyphenyl)methyl]-N-(3-[1-[2-(2,6-dioxopiperidin-3-yl)-1,3-dioxoisoindol-4-yl]azetidin-3-yl]propyl)carbamate (48.0 mg, 57.51%) as a yellow solid. LCMS (ESI) m/z [M+H]⁺=621.

### Step 6: Preparation of 4-[3-(3-aminopropyl)azetidin-1-yl]-2-(2,6-dioxopiperidin-3-yl)isoindole-1,3-dione (K-45)

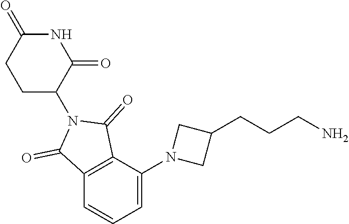 [SMILES: NCCCC1CN(c2cccc3c2C(=O)N(C2CCC(=O)NC2=O)C3=O)C1]

A solution of tert-butyl N-[(2,4-dimethoxyphenyl)methyl]-N-(3-[1-[2-(2,6-dioxopiperidin-3-yl)-1,3-dioxoisoindol-4-yl]azetidin-3-yl]propyl)carbamate (48.00 mg, 0.077 mmol, 1.00 equiv) in neat trifluoroacetic acid (1.00 mL) was stirred for 6 h at room temperature under a nitrogen atmosphere. The resulting mixture was concentrated under vacuum. The crude product was purified by flash chromatography with the following condition: column C18 spherical gel; Mobile Phase A: Water, Mobile Phase B: ACN; Flow rate: 40 mL/min; Gradient: 0% B to 100% B in 20 min; 254/220 nm. This resulted in 4-[3-(3-aminopropyl)azetidin-1-yl]-2-(2,6-dioxopiperidin-3-yl)isoindole-1,3- (21.0 mg, 73.31%) as a yellow solid. LCMS (ESI) m/z [M+H]⁺=371.

### Preparation of (2S,4R)-1-((S)-3,3-dimethyl-2-(2-oxoacetamido)butanoyl)-4-hydroxy-N-(4-(4-methylthiazol-5-yl)benzyl)pyrrolidine-2-carboxamide (K-46)

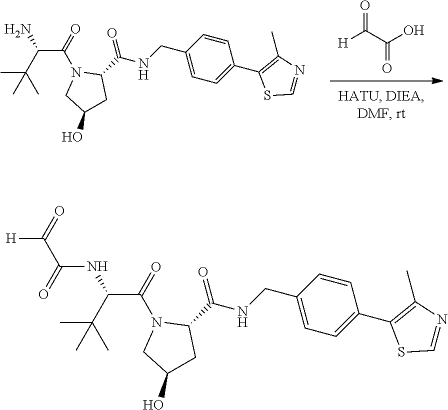 [SMILES: Cc1ncsc1-c1ccc(CNC(=O)[C@@H]2C[C@@H](O)CN2C(=O)[C@@H](N)C(C)(C)C)cc1 ; O=CC(=O)O ; Cc1ncsc1-c1ccc(CNC(=O)[C@@H]2C[C@@H](O)CN2C(=O)[C@@H](NC(=O)C=O)C(C)(C)C)cc1]

To a stirred solution of (2S,4R)-1-[(2S)-2-amino-3,3-dimethylbutanoyl]-4-hydroxy-N-[[4-(4-methyl-1,3-thiazol-5-yl)phenyl]methyl]pyrrolidine-2-carboxamide (50.00 mg, 0.116 mmol, 1.00 equiv) and glyoxalate (8.60 mg, 0.116 mmol, 1.00 equiv) in DMF (1.00 mL) was added HATU (66.23 mg, 0.174 mmol, 1.50 equiv) and DIEA (45.03 mg, 0.348 mmol, 3.00 equiv) at room temperature. The resulting mixture was stirred for 3 h at room temperature. The residue was purified by reverse flash chromatography (eluting with 0-100% acetonitrile/water with 0.1% formic acid) to provide the title compound (65 mg) as a yellow oil. LCMS (ESI) m/z: [M+H]⁺=487.

### Preparation of (2S,4R)-1-((S)-3,3-dimethyl-2-(11-oxoundecanamido)butanoyl)-4-hydroxy-N—((S)-1-(4-(4-methylthiazol-5-yl)phenyl)ethyl)pyrrolidine-2-carboxamide (K-47)

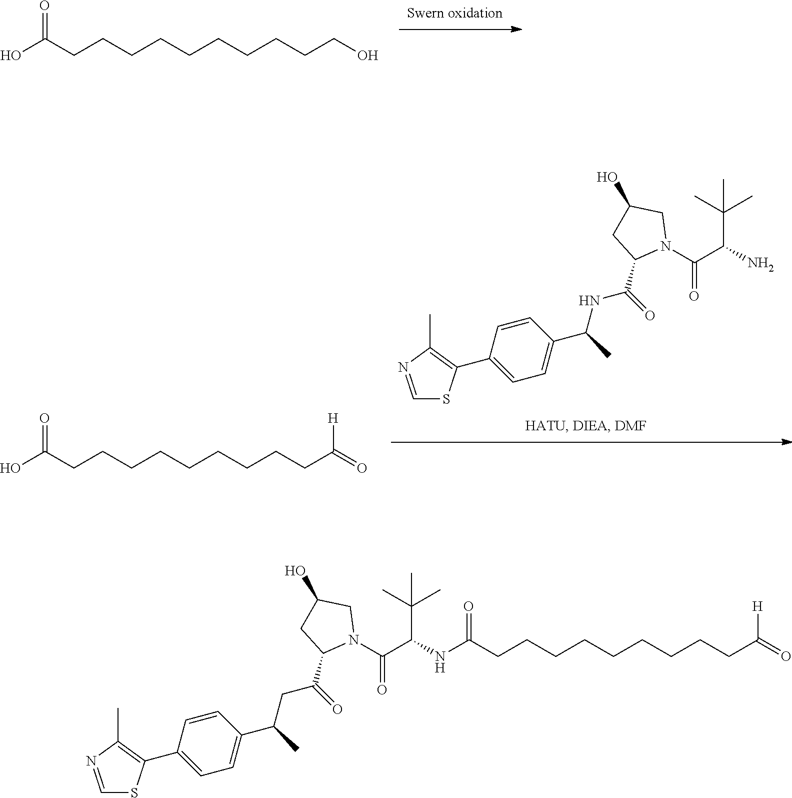 [SMILES: O=C(O)CCCCCCCCCCO ; Cc1ncsc1-c1ccc([C@H](C)NC(=O)[C@@H]2C[C@@H](O)CN2C(=O)[C@@H](N)C(C)(C)C)cc1 ; O=CCCCCCCCCCC(=O)O ; Cc1ncsc1-c1ccc([C@H](C)CC(=O)[C@@H]2C[C@@H](O)CN2C(=O)[C@@H](NC(=O)CCCCCCCCCC=O)C(C)(C)C)cc1]

### Step 1: Preparation of 11-oxoundecanoic acid

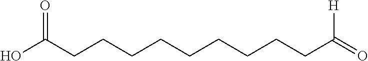 [SMILES: O=CCCCCCCCCCC(=O)O]

To a stirred solution of (COCl)₂ (2.51 g, 19.773 mmol, 4.00 equiv) in CH₂Cl₂ (10 mL) was added DMSO (1.16 g, 14.830 mmol, 3.00 equiv) dropwise at −78° C. under a nitrogen atmosphere. A solution of 11-hydroxyundecanoic acid (1.00 g, 4.943 mmol, 1.00 equiv) in CH₂Cl₂ (10 mL) was then added and the mixture was stirred for 30 min at degrees −60° C. under nitrogen atmosphere. Et₃N (2.50 g, 24.717 mmol, 5.00 equiv) was then added at −60° C. and the mixture was allowed to warm to room temperature and stirred for an additional 1.5 h. The resulting mixture was diluted with water (50 mL), then extracted with CH₂Cl₂ (50 mL×3). The combined organic layers were washed with brine (30 mL), then dried over anhydrous Na₂SO₄. After filtration, the filtrate was concentrated under reduced pressure. The crude product was used in the next step directly without further purification. This resulted in the title compound (950 mg, crude) as a yellow oil. LCMS (ESI) m/z: [M+H]⁺=200.

### Step 2: Preparation of (2S,4R)-1-((S)-3,3-dimethyl-2-(11-oxoundecanamido)butanoyl)-4-hydroxy-N—((S)-1-(4-(4-methylthiazol-5-yl)phenyl)ethyl)pyrrolidine-2-carboxamide (K-47)

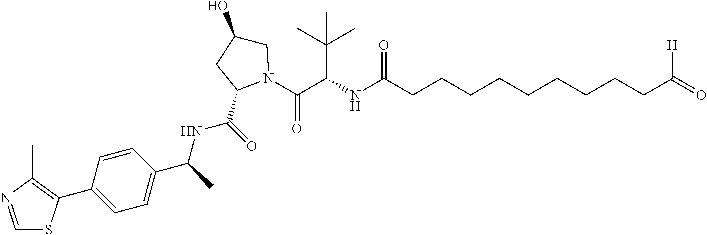 [SMILES: Cc1ncsc1-c1ccc([C@H](C)NC(=O)[C@@H]2C[C@@H](O)CN2C(=O)[C@@H](NC(=O)CCCCCCCCCC=O)C(C)(C)C)cc1]

To a stirred solution of 11-oxoundecanoic acid (100.00 mg, 0.499 mmol, 1.00 equiv) and (2S,4R)-1-[(2S)-2-amino-3,3-dimethylbutanoyl]-4-hydroxy-N-[(1S)-1-[4-(4-methyl-1,3-thiazol-5yl)phenyl]ethyl]pyrrolidine-2-carboxamide (266.38 mg, 1.20 equiv) in DMF (5 mL) was added HATU (284.78 mg, 0.748 mmol, 1.50 equiv) and DIEA (193.60 mg, 1.497 mmol, 3.00 equiv) in portions at room temperature. The resulting mixture was stirred for 6 h and then concentrated. The residue was purified by reverse flash chromatography to afford the title compound (90 mg, 28.74%) as a white solid. LCMS (ESI) m/z: [M+H]⁺=257.

### Preparation of (2S,4R)-1-((S)-2-acrylamido-3,3-dimethylbutanoyl)-4-hydroxy-N-(4-(4-methylthiazol-5-yl)benzyl)pyrrolidine-2-carboxamide (K-48)

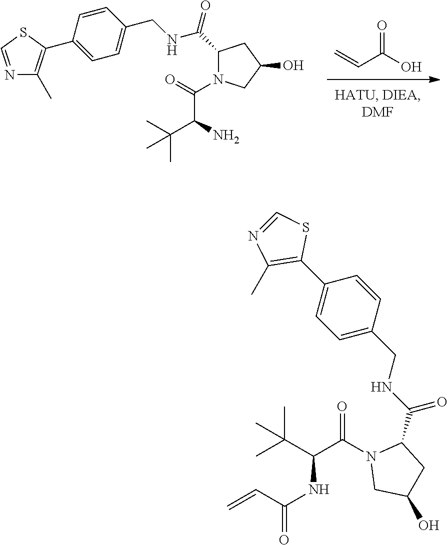

To a stirred RT mixture of (2S,4R)-1-[(2S)-2-amino-3,3-dimethylbutanoyl]-4-hydroxy-N-[[4-(4-methyl-1,3-thiazol-5-yl)phenyl]methyl]pyrrolidine-2-carboxamide (500.00 mg, 1.161 mmol, 1.00 equiv) and acrylic acid (83.68 mg, 1.161 mmol, 1.00 equiv) in DMF (4.00 mL) was added HATU (529.85 mg, 1.393 mmol, 1.20 equiv) and DIEA (450.25 mg, 3.483 mmol, 3.00 equiv). The resulting mixture was stirred for 2 h, then concentrated. The residue was purified by reverse flash chromatography (eluting with 10-50% acetonitrile in water over 30 min.), to provide the title compound (495 mg, 87.96%) as a yellow oil. LCMS (ESI) m/z [M+H]⁺=484.21.

### Preparation of (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (K-49)

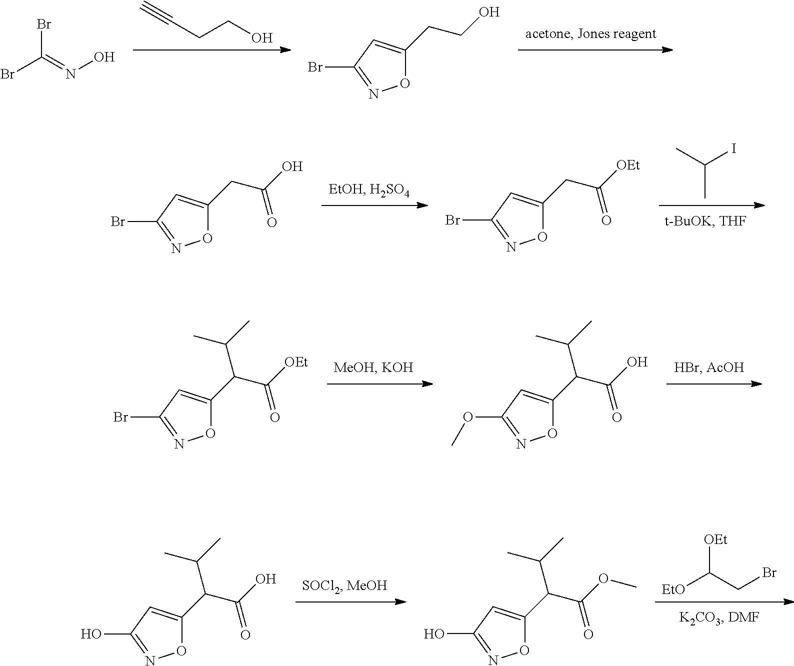

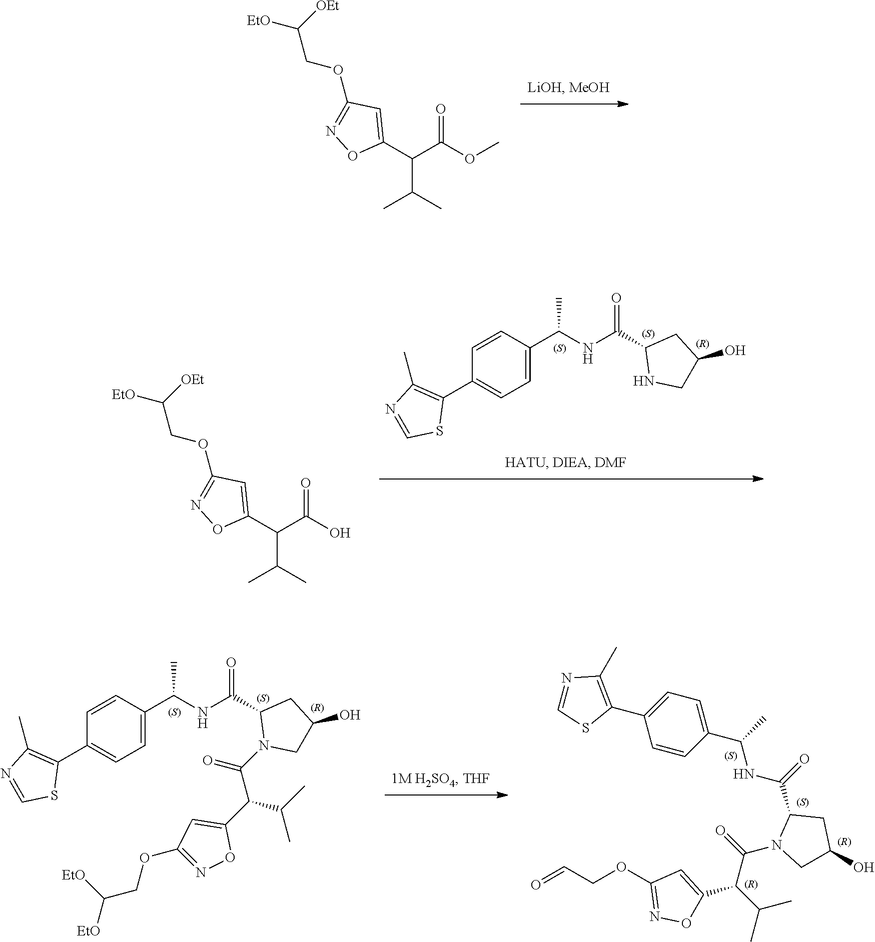

### Step 1: Preparation of 2-(3-bromoisoxazol-5-yl)ethan-1-ol

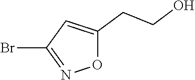

A solution of 3-butyn-1-ol (552.89 g, 7888.26 mmol, 4 equiv) and KHCO₃ (592.30 g, 5916.197 mmol, 3 equiv) in EtOAc (2600 mL) and H₂O (260 mL) was stirred at room temperature. 1-bromo-N-hydroxymethanecarbonimidoyl bromide (400.00 g in EA (840 mL), 1972.066 mmol, 1.00 equiv) was added dropwise over 60 min. The resulting mixture was stirred overnight at room temperature. The reaction mixture was washed with water (500 mL×2) and the organic layer was dried over anhydrous Na₂SO₄. After filtration, the filtrate was concentrated under reduced pressure. The residue was purified by silica gel column chromatography, eluting with PE/EtOAc (30:1) to afford the title compound (338.2 g, 88.98%) as an off-white solid. LCMS (ESI) m/z [M+H]⁺=192.

### Step 2: Preparation of 2-(3-bromoisoxazol-5-yl)acetic acid

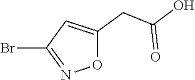

A solution of 2-(3-bromoisoxazol-5-yl)ethan-1-ol (360.00 g) in acetone (3600 mL) was stirred at 0 degrees C. under nitrogen atmosphere. To the above mixture was added Jones reagent (1760.00 mL) dropwise over 1 h at 0 degrees C. The resulting mixture was stirred overnight at room temperature. The reaction was quenched by the addition of ice water, and the resulting mixture was extracted with EtOAc (1000 mL×3). The combined organic layers were washed with water (500 mL×2) and the organic layer was dried over anhydrous Na₂SO₄. After filtration, the filtrate was concentrated under reduced pressure to afford the title compound (348.6 g, crude) as a green solid that was used directly without further purification. (LCMS (ESI) m/z [M+H]⁺=206.

### Step 3: Preparation of ethyl 2-(3-bromoisoxazol-5-yl)acetate

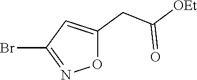 [SMILES: CCOC(=O)Cc1cc(Br)no1]

A solution of 2-(3-bromoisoxazol-5-yl)acetic acid (397.6 g, 1930.144 mmol, 1.00 equiv) and H₂SO₄ (18.92 g, 193.014 mmol, 0.1 equiv) in EtOH (2000 mL) was stirred for 2 h at 70 degrees C. The reaction mixture was concentrated under reduced pressure. The resulting mixture was diluted with EtOAc (3000 mL), washed with water (500 mL×2), and the organic layer was dried over anhydrous Na₂SO₄. After filtration, the filtrate was concentrated under reduced pressure. The residue was purified by silica gel column chromatography, eluting with PE/EtOAc (35:1), to afford the title compound (355 g, 78.61%) as a colorless oil. (LCMS (ESI) m/z [M+H]⁺=234.

### Step 4: Preparation of ethyl 2-(3-bromoisoxazol-5-yl)-3-methylbutanoate

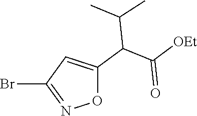

To a stirred solution of t-BuOK (244.51 g, 2179.031 mmol, 1.5 equiv) and ethyl 2-(3-bromoisoxazol-5-yl)acetate (340.00 g, 1452.687 mmol, 1.00 equiv) in THF (2000 mL) was added 2-iodopropane (321.03 g, 1888.493 mmol, 1.3 equiv) dropwise at 0 degrees C. under nitrogen atmosphere. The resulting mixture was stirred overnight at room temperature, then ice water was added. The mixture was extracted with EtOAc (1000 mL×2). The combined organic layers were washed with water (500 mL×1) and dried over anhydrous Na₂SO₄. After filtration, the filtrate was concentrated under reduced pressure. The residue was purified by silica gel column chromatography, eluting with PE/THF (10:1), to afford the title compound (284.1 g, 70.82%) as a colorless oil. (LCMS (ESI) m/z [M+H]⁺=276.

### Step 5: Preparation of 2-(3-methoxyisoxazol-5-yl)-3-methylbutanoic acid

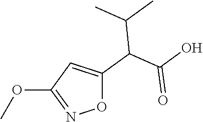

To a stirred solution of ethyl 2-(3-bromoisoxazol-5-yl)-3-methylbutanoate (90.00 g, 325.933 mmol, 1.00 equiv) in MeOH (270 mL) was added a solution of KOH (274.30 g, 4888.995 mmol, 15.00 equiv) in MeOH (210 mL) at 0 degrees C. The reaction mixture was stirred for overnight at 80 degrees C. The resulting solution was acidified to pH 4 with 1M solution of HCl (aq.) and concentrated under reduced pressure. The resulting mixture was diluted with EtOAc (1800 mL) and filtered. The filter cake was washed with EtOAc (100 mL×3). The filtrate was concentrated under reduced pressure to afford the title compound (62.9 g, 96.88%) as a yellow oil that was used directly without further purification. LCMS (ESI) m/z: [M+H]⁺=200.

### Step 6: Preparation of 2-(3-hydroxyisoxazol-5-yl)-3-methylbutanoic acid

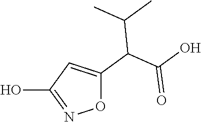

To a stirred solution of 2-(3-methoxyisoxazol-5-yl)-3-methylbutanoic acid (62.90 g, 315.754 mmol, 1.00 equiv) in HOAc (450.00 mL) was added 48% HBr (450.00 mL) at room temperature. The resulting mixture was stirred for 16 h at 60 degrees C., then concentrated under reduced pressure to give a residue. The residue was purified by flash C18-flash chromatography, eluting with 0 to 100% MeCN in water/0.05% formic acid), to afford the title compound (43.3 g, 74.05%) as a white solid. LCMS (ESI) m/z: [M+H]⁺=186.

### Step 7: Preparation of methyl 2-(3-hydroxyisoxazol-5-yl)-3-methylbutanoate

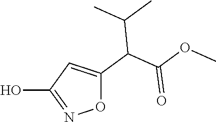

To a stirred solution of 2-(3-hydroxyisoxazol-5-yl)-3-methylbutanoic acid (20 g, 108.004 mmol, 1.00 equiv) in MeOH (72 mL) was added SOCl₂ (35.26 mL, 486.059 mmol, 4.50 equiv) at 0 degrees C. The resulting mixture was stirred for 16 h at room temperature, then concentrated under reduced pressure to give a residue. The residue was diluted with water (30 mL) and extracted with EtOAc (50 mL×3). The combined organic layers were washed with saturated brine (30 mL) and dried over anhydrous Na₂SO₄. After filtration, the filtrate was concentrated under reduced pressure. The crude product was purified by silica gel column chromatography, eluting with 0 to 100% THF in petroleum ether, to afford the title compound (15.1 g, 70.18%) as an off-white solid. ¹H NMR (400 MHz, DMSO-d₆) δ 11.24 (s, 1H), 5.95 (s, 1H), 3.71-3.58 (m, 4H), 2.32-2.20 (m, 1H), 0.88 (dd, J=34.2, 6.7 Hz, 6H). LCMS (ESI) m/z: [M+H]⁺=200.

### Step 8: Preparation of methyl 2-(3-(2,2-diethoxyethoxy)isoxazol-5-yl)-3-methylbutanoate

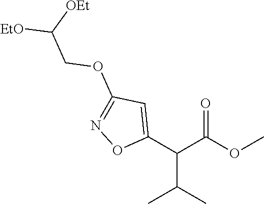 [SMILES: CCOC(COc1cc(C(C(=O)OC)C(C)C)on1)OCC]

To a stirred solution of methyl 2-(3-hydroxyisoxazol-5-yl)-3-methylbutanoate (7 g, 35.140 mmol, 1.00 equiv) and 2-bromo-1,1-diethoxyethane (7.62 g, 38.654 mmol, 1.1 equiv) in DMF (70 mL) was added K₂CO₃ (9.71 g, 70.280 mmol, 2 equiv). The resulting mixture was stirred for overnight at 80 degrees C. The resulting mixture was diluted with EtOAc (300 mL). The organic layer was washed with water (300 mL), brine (300 mL) then dried over anhydrous sodium sulfate, filtered and concentrated. The crude product was purified by preparative HPLC to afford the title compound (5.2 g, 46.92%) as a brown solid. LCMS (ESI) m/z: [M+H]⁺=316.

### Step 9: 2-(3-(2,2-diethoxyethoxy)isoxazol-5-yl)-3-methylbutanoic acid

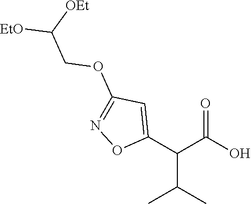 [SMILES: CCOC(COc1cc(C(C(=O)O)C(C)C)on1)OCC]

A solution of methyl 2-(3-(2,2-diethoxyethoxy)isoxazol-5-yl)-3-methylbutanoate (5.2 g, 16.489 mmol, 1.00 equiv) and LiOH (1.97 g, 82.445 mmol, 5 equiv) in MeOH (15 mL) and H₂O (45 mL) was prepared and the resulting mixture was stirred for 2 h at room temperature. The mixture was acidified to pH 5 with conc. HCl. The resulting mixture was extracted with EtOAc (300 mL×3). The combined organic layers were washed with brine (100 mL×1) and dried over anhydrous Na₂SO₄. After filtration, the filtrate was concentrated under reduced pressure and the filtrate was concentrated under vacuum. The crude product was used directly in the next step without further purification. LCMS (ESI) m/z: [M+H]⁺=302.

### Step 10: Preparation of (2S,4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide

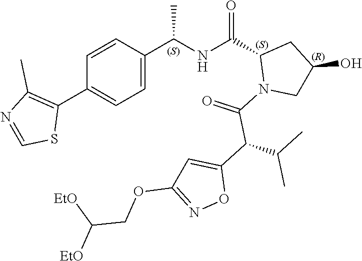

To a stirred solution of 2-(3-(2,2-diethoxyethoxy)isoxazol-5-yl)-3-methylbutanoic acid (4.95 g, 16.427 mmol, 1.00 equiv) and (2S,4R)-4-hydroxy-N-[(1S)-1-[4-(4-methyl-1,3-thiazol-5-yl)phenyl]ethyl]pyrrolidine-2-carboxamide (5.44 g, 16.427 mmol, 1 equiv) in DMF (50 mL) was added HATU (6.87 g, 18.070 mmol, 1.1 equiv) and DIEA (6.37 g, 49.281 mmol, 3 equiv). The resulting mixture was stirred for 2 h at room temperature. The resulting mixture was diluted with EtOAc (300 mL). The organic layer was washed with water (300 mL), brine (300 mL), then dried over anhydrous sodium sulfate, filtered and concentrated. The residue was purified by silica gel column chromatography, eluting with PE/EA (1:1) to afford crude product. The crude product (6.2 g) was purified by Chiral-HPLC, eluting with 40% methanol in carbon dioxide using a CHIRAL ART Amylose-SA column, to provide the title compound (2.8 g, 27.73%) as a white solid. 1H NMR (400 MHz, DMSO-d6) δ 8.99 (d, J=3.4 Hz, 1H), 8.43 (d, J=7.7 Hz, 1H), 7.49-7.41 (m, 2H), 7.41-7.31 (m, 2H), 6.14 (s, 1H), 5.10 (d, J=3.6 Hz, 1H), 4.97-4.87 (m, 1H), 4.81 (t, J=5.2 Hz, 1H), 4.37 (t, J=7.9 Hz, 1H), 4.32-4.23 (m, 1H), 4.09 (d, J=5.3 Hz, 2H), 3.73-3.49 (m, 6H), 3.45 (d, J=10.8 Hz, 1H), 2.46 (d, J=2.1 Hz, 3H), 2.31-2.15 (m, 1H), 2.03 (ddd, J=11.9, 8.1, 3.0 Hz, 1H), 1.78 (ddd, J=12.8, 8.0, 4.7 Hz, 1H), 1.41 (dd, J=29.6, 7.0 Hz, 3H), 1.13 (t, J=7.0 Hz, 6H), 0.96 (t, J=6.4 Hz, 3H), 0.81 (dd, J=14.4, 6.7 Hz, 3H). LCMS (ESI) m/z: [M+H]⁺=615.35.

### Step 11: Preparation of (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (K-49)

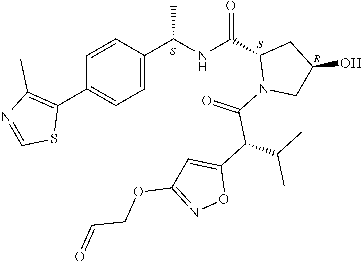

To a stirred solution of H₂SO₄ (1M) (6.00 mL) and THF (6.00 mL) was added (2S,4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide (300.00 mg, 0.499 mmol, 1.00 equiv) at room temperature. The resulting mixture was stirred for 8 h at 50 degrees C. The reaction was quenched by the addition of ice water, and the mixture was basified to pH 7 with saturated NaHCO₃ (aq.). The resulting mixture was extracted with EtOAc (3×100 mL). The combined organic layers were washed with brine (2×100 mL) and dried over anhydrous Na₂SO₄. After filtration, the filtrate was concentrated under reduced pressure. to afford (2S,4R)-4-hydroxy-N-[(1S)-1-[4-(4-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide (256 mg, 97.3%) as a white solid that was used directly without further purification. LCMS (ESI) m/z: [M+H]⁺=541.

### Preparation of 4-(2-((3-amino-6-(2-hydroxyphenyl)pyridazin-4-yl)oxy)ethyl)benzoic acid (I-64)

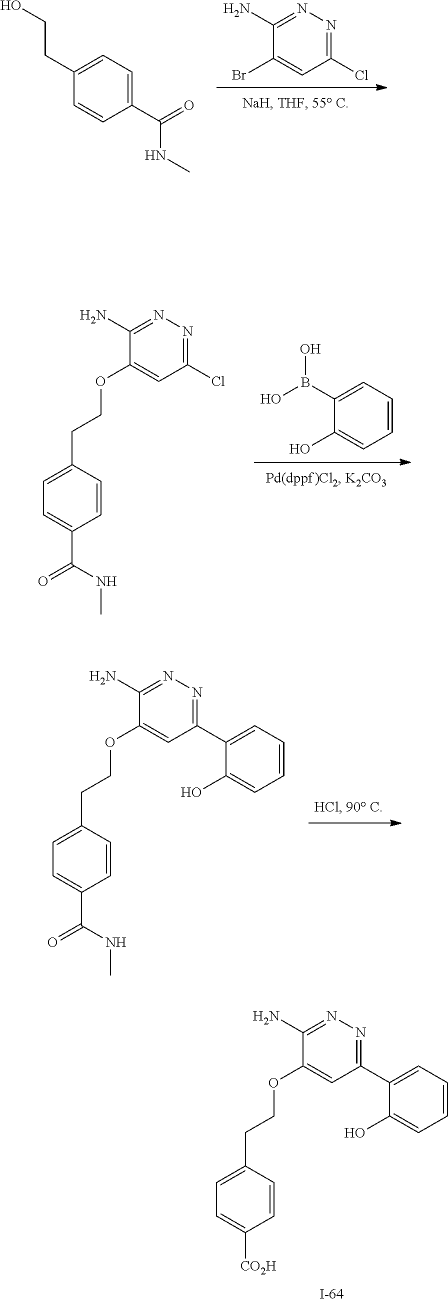

### Step 1: Preparation of 4-(2-((3-amino-6-chloropyridazin-4-yl)oxy)ethyl)-N-methylbenzamide

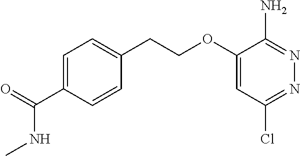 [SMILES: CNC(=O)c1ccc(CCOc2cc(Cl)nnc2N)cc1]

To a solution of 4-(2-hydroxyethyl)-N-methylbenzamide (1.00 g, 5.580 mmol, 1.00 equiv) in THF (30.00 mL) was added NaH (333.33 mg, 8.370 mmol, 1.50 equiv, 60% in oil) in portions at 0° C. under an atmosphere of dry nitrogen. The resulting mixture was stirred at 0° C. for 30 minutes and 4-bromo-6-chloropyridazin-3-amine (1.40 g, 6.696 mmol, 1.20 equiv) was added at room temperature. The mixture was heated to 60° C. and stirred for 6 h. The mixture was poured into saturated aqueous NH₄Cl (20 mL) and extracted with DCM (3×50 mL). The combined organic layers were dried over anhydrous Na₂SO₄, filtered and concentrated, and the residue was purified by silica gel column chromatography, eluting with DCM to 20:1 DCM/MeOH to afford 4-(2-((3-amino-6-chloropyridazin-4-yl)oxy)ethyl)-N-methylbenzamide (546 mg, 28.39%) as a light yellow solid. LCMS (ESI) m/z: [M+H]⁺=307.

### Step 2: Preparation of 4-(2-((3-amino-6-(2-hydroxyphenyl)pyridazin-4-yl)oxy)ethyl)-N-methylbenzamide

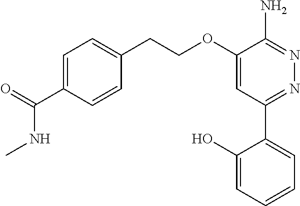 [SMILES: CNC(=O)c1ccc(CCOc2cc(-c3ccccc3O)nnc2N)cc1]

A mixture of 4-(2-((3-amino-6-chloropyridazin-4-yl)oxy)ethyl)-N-methylbenzamide (546.00 mg, 1.780 mmol, 1.00 equiv) and 2-hydroxyphenylboronic acid (368.26 mg, 2.670 mmol, 1.50 equiv) in 4:1 dioxane/water (10.00 mL) was sparged for 5 minutes with nitrogen. Pd(dppf)Cl₂ (130.24 mg, 0.178 mmol, 0.1 equiv) and K₂CO₃ (45.05 mg, 0.326 mmol, 2.0 equiv) were added. The resulting mixture was stirred at 80° C. for 4 h. The mixture was cooled to room temperature and quenched with water. The resulting mixture was extracted with EtOAc (3×50 mL). The combined organic layers were washed with brine (20 mL), dried over anhydrous Na₂SO₄. After filtration, the filtrate was concentrated under reduced pressure and purified to afford 4-(2-((3-amino-6-(2-hydroxyphenyl)pyridazin-4-yl)oxy)ethyl)-N-methylbenzamide (429 mg, 62.83%) as a brown solid. LCMS (ESI) m/z [M+H]⁺=365.

### Step 3: Preparation of 4-(2-((3-amino-6-(2-hydroxyphenyl)pyridazin-4-yl)oxy)ethyl)benzoic acid (I-64)

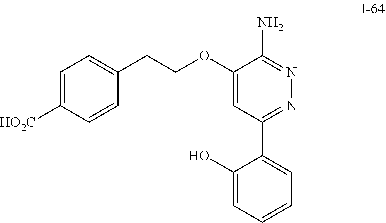 [SMILES: Nc1nnc(-c2ccccc2O)cc1OCCc1ccc(C(=O)O)cc1]

A mixture of 4-(2-((3-amino-6-(2-hydroxyphenyl)pyridazin-4-yl)oxy)ethyl)-N-methylbenzamide (429.00 mg, 1.179 mmol, 1.00 equiv) in concentrated HCl (10.00 mL, 12 M) was stirred at 90° C. for 8 h. The mixture was cooled to room temperature, diluted with water (5 mL), neutralized with NaHCO₃ to pH 3, and filtered. The filter cake was further purified by reversed-phase preparative HPLC to afford 4-(2-((3-amino-6-(2-hydroxyphenyl)pyridazin-4-yl)oxy)ethyl)benzoic acid (I-64, 389 mg, 90.91%) as a white solid. LCMS (ESI) m/z [M+H]⁺=352.20.

### Preparation of (4-(2-((3-amino-6-(2-hydroxyphenyl)pyridazin-4-yl)oxy)ethyl)phenyl)(3-(piperidin-4-yl)azetidin-1-yl)methanone (K-50)

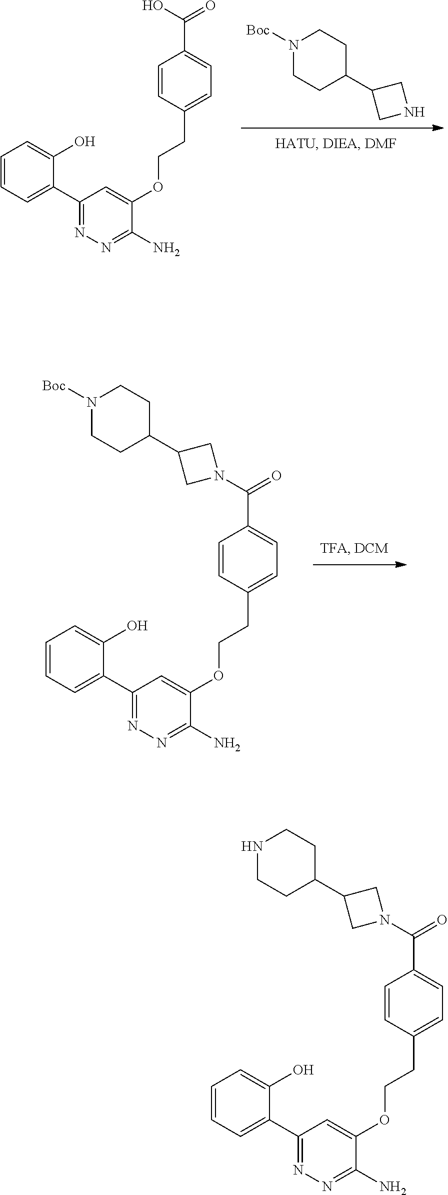 [SMILES: Nc1nnc(-c2ccccc2O)cc1OCCc1ccc(C(=O)O)cc1 ; CC(C)(C)OC(=O)N1CCC(C2CNC2)CC1 ; CC(C)(C)OC(=O)N1CCC(C2CN(C(=O)c3ccc(CCOc4cc(-c5ccccc5O)nnc4N)cc3)C2)CC1 ; Nc1nnc(-c2ccccc2O)cc1OCCc1ccc(C(=O)N2CC(C3CCNCC3)C2)cc1]

### Step 1: Preparation of tert-butyl 4-[1-[4-(2-[[3-amino-6-(2-hydroxyphenyl)pyridazin-4-yl]oxy]ethyl)benzoyl]azetidin-3-yl]piperidine-1-carboxylate

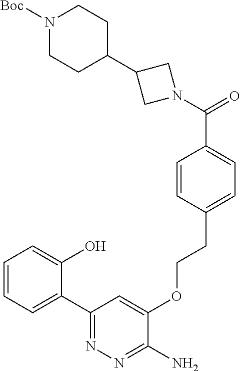 [SMILES: CC(C)(C)OC(=O)N1CCC(C2CN(C(=O)c3ccc(CCOc4cc(-c5ccccc5O)nnc4N)cc3)C2)CC1]

To a stirred solution of 4-(2-[[3-amino-6-(2-hydroxyphenyl)pyridazin-4-yl]oxy]ethyl)benzoic acid (50.0 mg, 0.142 mmol, 1.00 equiv) and tert-butyl 4-(azetidin-3-yl)piperidine-1-carboxylate (34.2 mg, 0.142 mmol, 1.00 equiv) in DMF (2.0 mL) were added HATU (64.9 mg, 0.171 mmol, 1.20 equiv) and DIEA (36.7 mg, 0.285 mmol, 2.00 equiv) at room temperature. The resulting mixture was stirred for 2 h at room temperature. The mixture was purified by reverse flash chromatography with the following conditions (column, C18 silica gel; mobile phase, MeCN in water (0.1% FA), 0 to 100% gradient in 30 min; detector, UV 254/220 nm). This resulted in the title compound (55 mg, 57.37%) as an off-white solid. LCMS (ESI) m/z: [M+H]⁺=574.

### Step 2: Preparation of 2-[6-amino-5-(2-[4-[3-(piperidin-4-yl)azetidine-1-carbonyl]phenyl]ethoxy)pyridazin-3-yl]phenol (K-50)

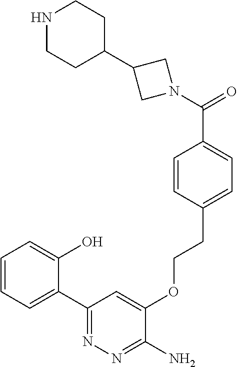 [SMILES: Nc1nnc(-c2ccccc2O)cc1OCCc1ccc(C(=O)N2CC(C3CCNCC3)C2)cc1]

To a stirred solution of tert-butyl 4-[1-[4-(2-[[3-amino-6-(2-hydroxyphenyl)pyridazin-4-yl]oxy]ethyl)benzoyl]azetidin-3-yl]piperidine-1-carboxylate (55.0 mg, 0.096 mmol, 1.00 equiv) in DCM (2.0 mL) was added TFA (1.0 mL) at room temperature. The resulting solution was stirred for 2 h and the resulting mixture was concentrated under reduced pressure. The residue was purified by reverse flash chromatography with the following conditions (column, C18 silica gel; mobile phase, MeCN in water (0.1% FA), 0 to 100% gradient in 30 min; detector, UV 254/220 nm). This resulted in the title compound (20 mg, 44.05%) as a pink solid. LCMS (ESI) m/z: [M+H]⁺=474.

The following intermediates in Table A13 were prepared in a similar manner as described in the preparation of K-50.

TABLE A13

| | | | LCMS |
|---|---|---|---|
| | Intermediate | | (ESI)m/z: |
| Structure | No. | Name | [M + H]⁺ |
| 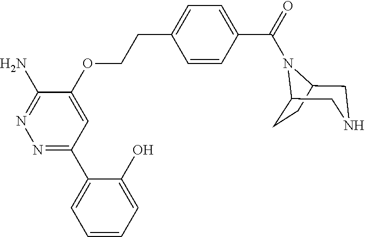 [SMILES: Nc1nnc(-c2ccccc2O)cc1OCCc1ccc(C(=O)N2C3CCC2CNC3)cc1] | K-51 | (4-(2-((3-amino-6-(2- hydroxyphenyl)pyridazin-4- yl)oxy)ethyl)phenyl)(3,8- diazabicyclo[3.2.1]octan-8- yl)methanone | 445.5 |
| 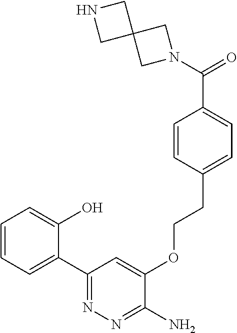 [SMILES: Nc1nnc(-c2ccccc2O)cc1OCCc1ccc(C(=O)N2CC3(CNC3)C2)cc1] | K-52 | (4-(2-((3-amino-6-(2- hydroxyphenyl)pyridazin-4- yl)oxy)ethyl)phenyl)(2,6- diazaspiro[3.3]heptan-2- yl)methanone | 432.0 |
| 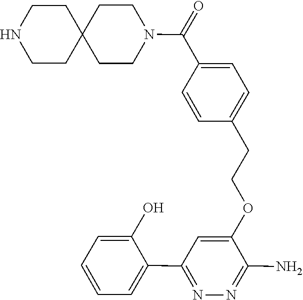 [SMILES: Nc1nnc(-c2ccccc2O)cc1OCCc1ccc(C(=O)N2CCC3(CCNCC3)CC2)cc1] | K-53 | (4-(2-((3-amino-6-(2- hydroxyphenyl)pyridazin-4- yl)oxy)ethyl)phenyl)(3,9- diazaspiro[5.5]undecan-3- yl)methanone | 488 |
| 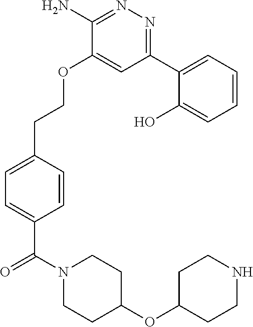 [SMILES: Nc1nnc(-c2ccccc2O)cc1OCCc1ccc(C(=O)N2CCC(OC3CCNCC3)CC2)cc1] | K-54 | (4-(2-((3-amino-6-(2- hydroxyphenyl)pyridazin-4- yl)oxy)ethyl)phenyl)(4-(piperidin-4- yloxy)piperidin-1-yl)methanone | 517.6 |
| 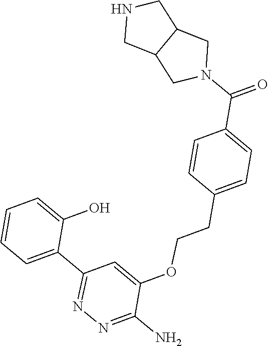 [SMILES: Nc1nnc(-c2ccccc2O)cc1OCCc1ccc(C(=O)N2CC3CNCC3C2)cc1] | K-55 | (4-(2-((3-amino-6-(2- hydroxyphenyl)pyridazin-4- yl)oxy)ethyl)phenyl)(hexahydropyrr olo[3,4-c]pyrrol-2(1H)- yl)methanone | 446.0 |
| 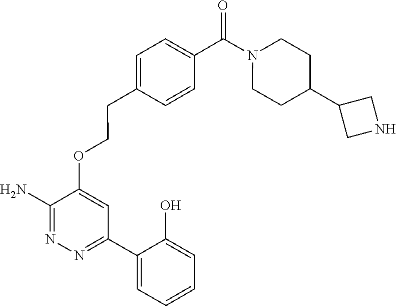 [SMILES: Nc1nnc(-c2ccccc2O)cc1OCCc1ccc(C(=O)N2CCC(C3CNC3)CC2)cc1] | K-56 | (4-(2-((3-amino-6-(2- hydroxyphenyl)pyridazin-4- yl)oxy)ethyl)phenyl)(4-(azetidin-3- yl)piperidin-1-yl)methanone | 474 |
| 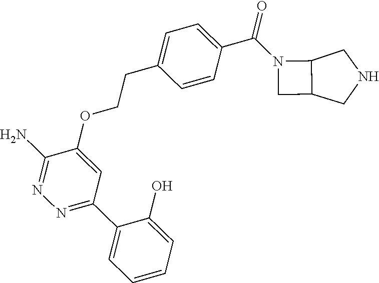 [SMILES: Nc1nnc(-c2ccccc2O)cc1OCCc1ccc(C(=O)N2CC3CNCC32)cc1] | K-57 | (4-(2-((3-amino-6-(2- hydroxyphenyl)pyridazin-4- yl)oxy)ethyl)phenyl)(3,6- diazabicyclo[3.2.0]heptan-6- yl)methanone | 432 |
| 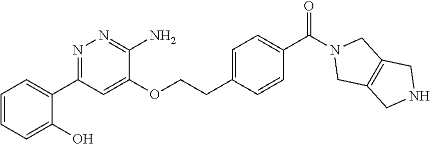 [SMILES: Nc1nnc(-c2ccccc2O)cc1OCCc1ccc(C(=O)N2CC3=C(CNC3)C2)cc1] | K-58 | (4-(2-((3-amino-6-(2- hydroxyphenyl)pyridazin-4- yl)oxy)ethyl)phenyl)(3,4,5,6- tetrahydropyrrolo[3,4-c]pyrrol- 2(1H)-yl)methanone | 444 |
| 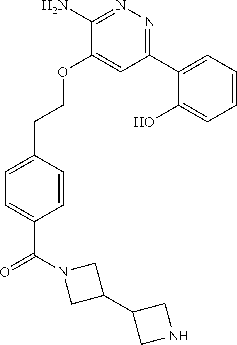 [SMILES: Nc1nnc(-c2ccccc2O)cc1OCCc1ccc(C(=O)N2CC(C3CNC3)C2)cc1] | K-59 | [3,3′-biazetidin]-1-yl(4-(2-((3- amino-6-(2- hydroxyphenyl)pyridazin-4- yl)oxy)ethyl)phenyl)methanone | 446.2 |
| 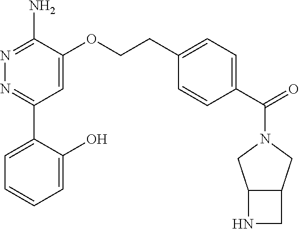 [SMILES: Nc1nnc(-c2ccccc2O)cc1OCCc1ccc(C(=O)N2CC3CNC3C2)cc1] | K-60 | (4-(2-((3-amino-6-(2- hydroxyphenyl)pyridazin-4- yl)oxy)ethyl)phenyl)(3,6- diazabicyclo[3.2.0]heptan-3- yl)methanone | 432.2 |
| 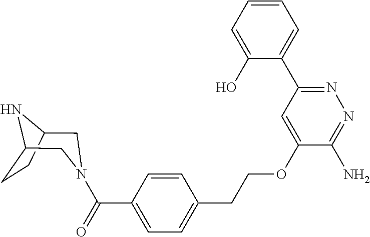 [SMILES: Nc1nnc(-c2ccccc2O)cc1OCCc1ccc(C(=O)N2CC3CCC(C2)N3)cc1] | K-61 | (4-(2-((3-amino-6-(2- hydroxyphenyl)pyridazin-4- yl)oxy)ethyl)phenyl)(3,8- diazabicyclo[3.2.1]octan-3- yl)methanone | 446.0 |

### Preparation of 2-(6-amino-5-[2-[4-(aminomethyl)phenyl]ethoxy]pyridazin-3-yl)phenol FA (I-65)

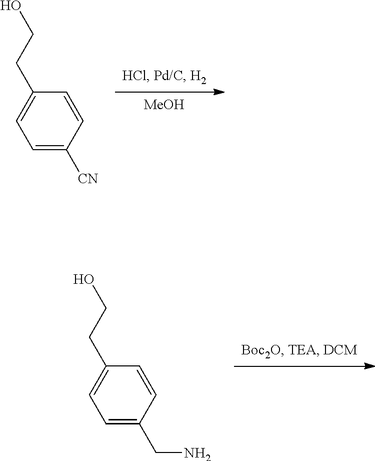 [SMILES: "MeOH" ; N#Cc1ccc(CCO)cc1 ; NCc1ccc(CCO)cc1]

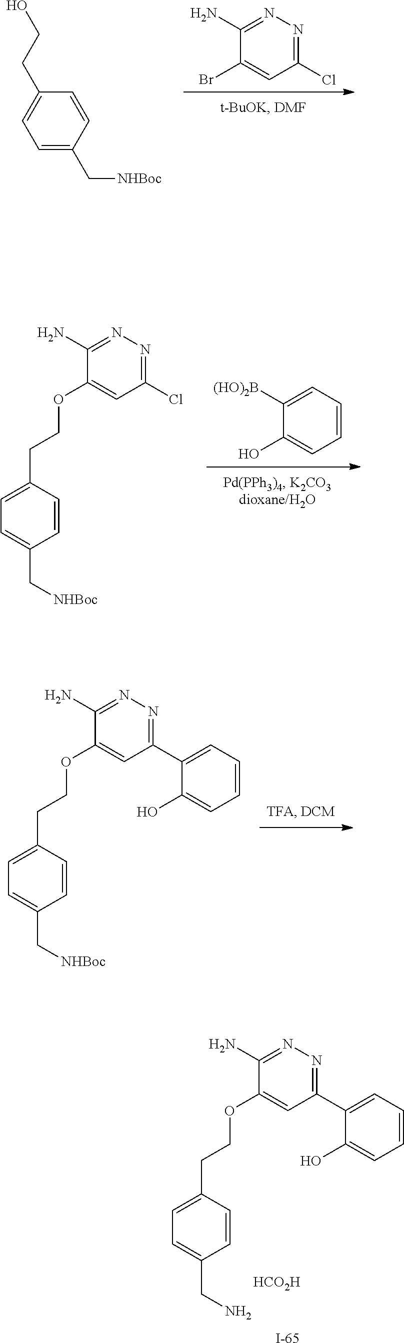

### Step 1: Preparation of 2-(4-(aminomethyl)phenyl)ethan-1-ol

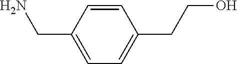 [SMILES: NCc1ccc(CCO)cc1]

To a stirred mixture of 4-(2-hydroxyethyl)benzonitrile (2.00 g, 13.589 mmol, 1.00 equiv) in MeOH (50.0 mL) were added concentrated HCl (1.0 mL) and wet 10% Pd/C (1.00 g) at room temperature. The reaction vessel was purged with hydrogen three times and the resulting mixture was stirred for 16 h at room temperature under 5 atm of hydrogen. The reaction vessel was sparged with nitrogen, and the catalyst removed by filtration through Celite®. The filter cake was washed with MeOH and the filtrate was concentrated in vacuum to afford of 2-(4-(aminomethyl)phenyl)ethan-1-ol (2.08 g, quantitative) as a light brown solid that was used in the next step without further purification. LCMS (ESI) m/z [M+H]⁺=152.

### Step 2: Preparation of tert-butyl (4-(2-hydroxyethyl)benzyl)carbamate

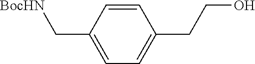 [SMILES: CC(C)(C)OC(=O)NCc1ccc(CCO)cc1]

To a stirred mixture of 2-(4-(aminomethyl)phenyl)ethan-1-ol (2.00 g, 1.00 equiv, 13.23 mmol) in DCM (30.00 mL) were added Boc₂O (4.33 g, 1.50 equiv, 19.840 mmol) and TEA (2.677 g, 2.00 equiv, 26.454 mmol) in portions at 0° C. The reaction mixture was stirred for 4 h at room temperature before quenching with water (10 mL). The resulting mixture was extracted with DCM (3×50 mL) and the combined organic layers were washed with brine (10 mL), dried over anhydrous Na₂SO₄, filtered, and concentrated under reduced pressure. The residue was purified by reversed phase flash chromatography (C18 column; mobile phase, ACN in water, 0% to 80% gradient in 30 min; detector, UV 254 nm) to afford tert-butyl (4-(2-hydroxyethyl)benzyl)carbamate (2.14 g, 64.46%) as a white solid. LCMS (ESI) m/z [M+H]⁺=252.

### Step 3: Preparation of tert-butyl N-[(4-[2-[(3-amino-6-chloropyridazin-4-yl)oxy]ethyl]phenyl)methyl]carbamate

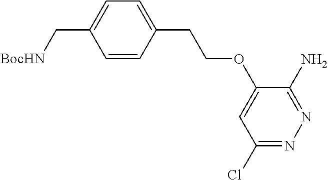 [SMILES: CC(C)(C)OC(=O)NCc1ccc(CCOc2cc(Cl)nnc2N)cc1]

To a stirred mixture of tert-butyl (4-(2-hydroxyethyl)benzyl)carbamate (2.14 g, 8.53 mmol, 1.00 equiv) in DMF (20 mL) was added t-BuOK (1.43 g, 12.79 mmol, 1.50 equiv) in portions at 0° C. The reaction mixture was stirred for 45 min at 0° C. A solution of 4-bromo-6-chloropyridazin-3-amine (1.78 g, 8.53 mmol, 1.00 equiv) in anhydrous DMF (10 mL) was added dropwise at room temperature. The reaction mixture was heated at 60° C. for 4 h before quenching with water (50 mL). The resulting mixture was extracted with DCM (3×50 mL) and the combined organic layers were washed with brine (100 mL), dried over anhydrous Na₂SO₄, filtered, and concentrated under reduced pressure. The residue was purified by reversed phase flash chromatography to afford tert-butyl N-[(4-[2-[(3-amino-6-chloropyridazin-4-yl)oxy]ethyl]phenyl)methyl]carbamate (1.65 g, 51.17%) as a brown solid. LCMS (ESI) m/z [M+H]⁺=379.

### Step 4: Preparation of tert-butyl N-[[4-(2-[[3-amino-6-(2-hydroxyphenyl)pyridazin-4-yl]oxy]ethyl)phenyl]methyl]carbamate

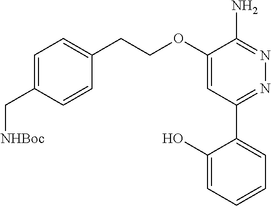 [SMILES: CC(C)(C)OC(=O)NCc1ccc(CCOc2cc(-c3ccccc3O)nnc2N)cc1]

To a stirred mixture of tert-butyl N-[(4-[2-[(3-amino-6-chloropyridazin-4-yl)oxy]ethyl]phenyl)methyl]carbamate (1.00 g, 2.640 mmol, 1.00 equiv) and 2-hydroxyphenylboronic acid (545 mg, 3.960 mmol, 1.50 equiv) in dioxane (12.0 mL) and water (3.0 mL) were added K₂CO₃ (0.729 g, 5.280 mmol, 2.0 equiv) and Pd(PPh₃)₄ (305.184 mg, 0.264 mmol, 0.10 equiv) at room temperature under an atmosphere of dry nitrogen. The resulting mixture was stirred for 4 h at 80° C. under an atmosphere of dry nitrogen. The mixture was allowed to cool down to room temperature and diluted with water (10 mL). The resulting mixture was extracted with EtOAc (3×50 mL). The combined organic layers were dried over anhydrous Na₂SO₄, filtered, and concentrated under reduced pressure. The residue was purified by silica gel column chromatography to afford tert-butyl N-[[4-(2-[[3-amino-6-(2-hydroxyphenyl)pyridazin-4-yl]oxy]ethyl)phenyl]methyl]carbamate (765 mg, 66.46%) as a white solid. LCMS (ESI) m/z [M+H]⁺=437.

### Step 5: Preparation of 2-(6-amino-5-[2-[4-(aminomethyl)phenyl]ethoxy]pyridazin-3-yl)phenol FA (I-65)

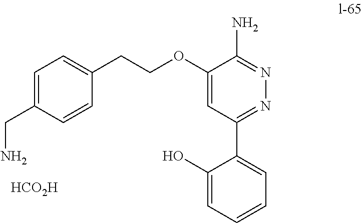 [SMILES: "HCO2H" ; NCc1ccc(CCOc2cc(-c3ccccc3O)nnc2N)cc1]

To a stirred mixture of tert-butyl N-[[4-(2-[[3-amino-6-(2-hydroxyphenyl)pyridazin-4-yl]oxy]ethyl)phenyl]methyl]carbamate (200.00 mg, 0.459 mmol, 1.00 equiv) in DCM (10 mL) was added TFA (2 mL) dropwise at room temperature under an atmosphere of dry nitrogen. The resulting mixture was stirred for 1 h at room temperature, then concentrated under reduced pressure. The residue was purified by reversed phase flash chromatography to afford intermediate I-65 (98.00 mg, 55.9%) as a white solid. LCMS (ESI) m/z [M+H]⁺=337.25.

The following intermediates in Table A14 were prepared in a similar manner as described in the preparation of I-65.

TABLE A14

| | | | LCMS |
|---|---|---|---|
| | Intermediate | | (ESI)m/z: |
| Structure | No. | Name | [M + H]⁺ |
| 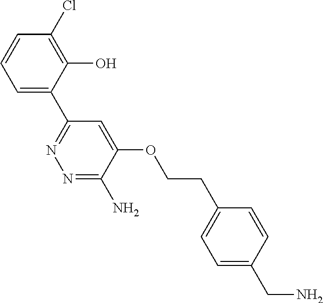 [SMILES: NCc1ccc(CCOc2cc(-c3cccc(Cl)c3O)nnc2N)cc1] | K-62 | 2-(6-amino-5-(4- (aminomethyl)phenethoxy) pyridazin-3-yl)-6-chlorophenol | 371 |
| 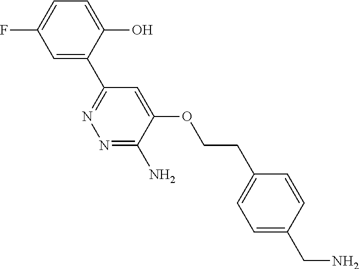 [SMILES: NCc1ccc(CCOc2cc(-c3cc(F)ccc3O)nnc2N)cc1] | K-63 | 2-(6-amino-5-(4- (aminomethyl)phenethoxy) pyridazin-3-yl)-4-fluorophenol | 355 |
| 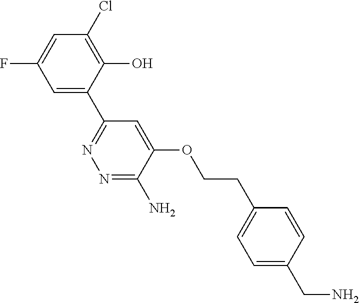 [SMILES: NCc1ccc(CCOc2cc(-c3cc(F)cc(Cl)c3O)nnc2N)cc1] | K-64 | 2-(6-amino-5-(4- (aminomethyl)phenethoxy) pyridazin-3-yl)-6-chloro- 4-fluorophenol | 389 |

### Preparation of N-(4-(2-((3-amino-6-(2-hydroxyphenyl)pyridazin-4-yl)oxy)ethyl)benzyl)-2-(2,6-diazaspiro[3.3]heptan-2-yl)acetamide (K-65)

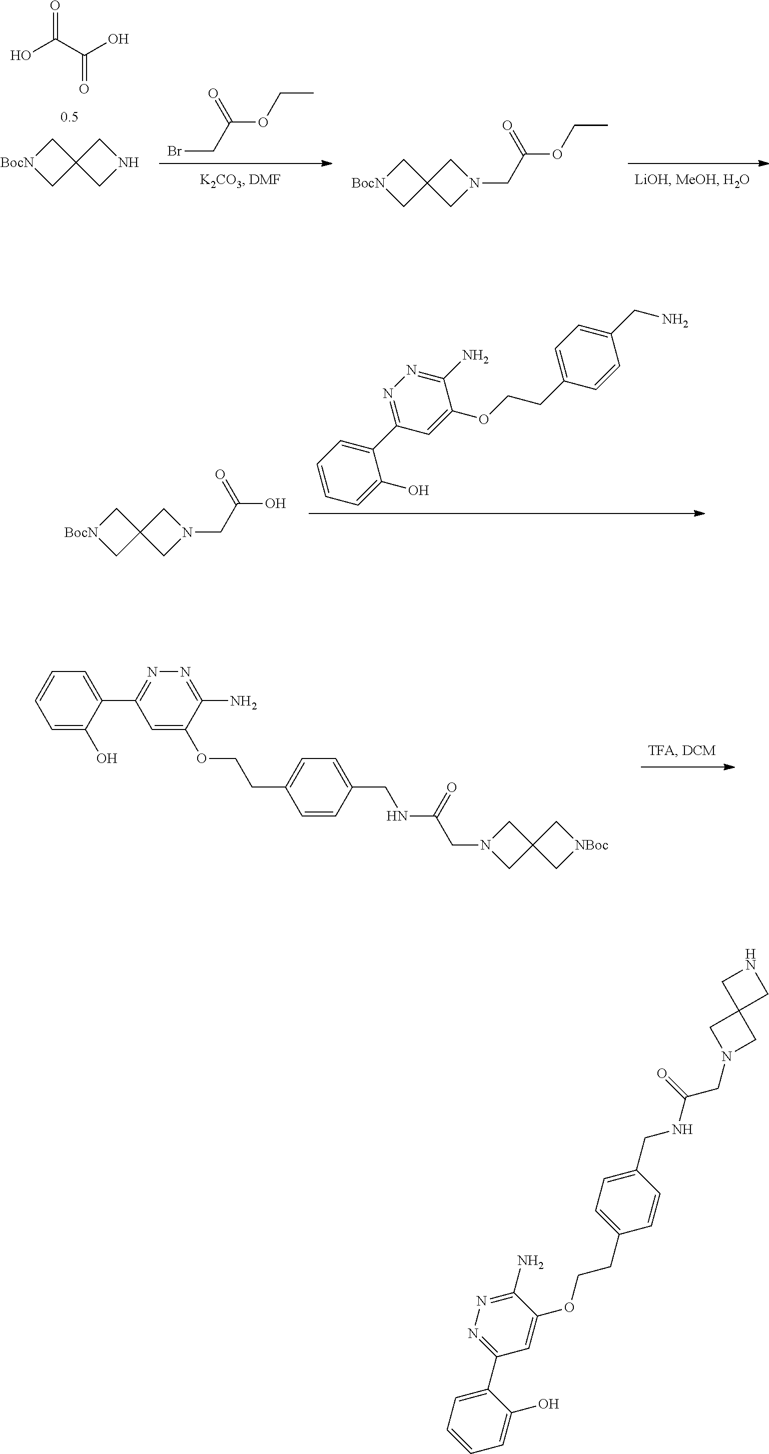

### Step 1: Preparation of tert-butyl 6-(2-ethoxy-2-oxoethyl)-2,6-diazaspiro[3.3]heptane-2-carboxylate

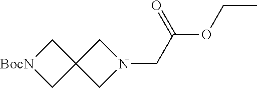 [SMILES: CCOC(=O)CN1CC2(C1)CN(C(=O)OC(C)(C)C)C2]

To a stirred solution of tert-butyl 2,6-diazaspiro[3.3]heptane-2-carboxylate hemi-oxalate (200.00 mg, 0.694 mmol, 1.00 equiv) and ethyl bromoacetate (139.02 mg, 0.832 mmol, 1.20 equiv) in DMF (4.00 mL) were added K₂CO₃ (239.69 mg, 1.734 mmol, 2.5 equiv) at room temperature. The resulting mixture was stirred for 2 h at 50 degrees C. The residue was purified by reverse flash chromatography to afford the title compound (280 mg, crude) as a white solid. LCMS (ESI) m/z: [M+H]⁺=285.

### Step 2: Preparation of 2-(6-(tert-butoxycarbonyl)-2,6-diazaspiro[3.3]heptan-2-yl)acetic acid

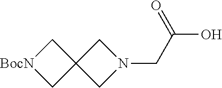 [SMILES: CC(C)(C)OC(=O)N1CC2(CN(CC(=O)O)C2)C1]

A solution of tert-butyl 6-(2-ethoxy-2-oxoethyl)-2,6-diazaspiro[3.3]heptane-2-carboxylate (200.00 mg, 0.703 mmol, 1.00 equiv) and LiOH (67.37 mg, 2.813 mmol, 4.00 equiv) in MeOH (2.00 mL) and H₂O (0.50 mL) was stirred for 2 h at 60 degrees C. The resulting mixture was concentrated under reduced pressure to afford the title compound (250 mg, crude) as a white solid. LCMS (ESI) m/z: [M+H]⁺=257.

### Step 3: Preparation of tert-butyl 6-(2-((4-(2-((3-amino-6-(2-hydroxyphenyl)pyridazin-4-yl)oxy)ethyl)benzyl)amino)-2-oxoethyl)-2,6-diazaspiro[3.3]heptane-2-carboxylate

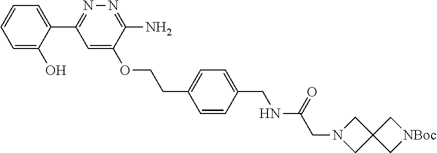 [SMILES: CC(C)(C)OC(=O)N1CC2(CN(CC(=O)NCc3ccc(CCOc4cc(-c5ccccc5O)nnc4N)cc3)C2)C1]

To a stirred solution of 2-(6-amino-5-(4-(aminomethyl)phenethoxy)pyridazin-3-yl)phenol (150.00 mg, 0.585 mmol, 1.00 equiv) and 2-(6-(tert-butoxycarbonyl)-2,6-diazaspiro[3.3]heptan-2-yl)acetic acid (196.87 mg, 0.585 mmol, 1.00 equiv) in DMF (4.00 mL) were added HATU (222.53 mg, 0.585 mmol, 1 equiv) and DIEA (226.92 mg, 1.756 mmol, 3 equiv) at room temperature. The resulting mixture was stirred for 2 h at room temperature. The residue was purified by reverse flash chromatography to afford the title compound (37 mg, 11.00%) as a white solid. LCMS (ESI) m/z: [M+H]⁺=575.

### Step 4: Preparation of N-[[4-(2-[[3-amino-6-(2-hydroxyphenyl)pyridazin-4-yl]oxy]ethyl)phenyl]methyl]-2-[2,6-diazaspiro[3.3]heptan-2-yl]acetamide (K-65)

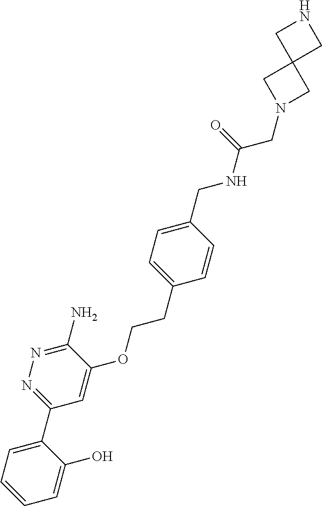 [SMILES: Nc1nnc(-c2ccccc2O)cc1OCCc1ccc(CNC(=O)CN2CC3(CNC3)C2)cc1]

To a stirred solution of tert-butyl 6-(2-((4-(2-((3-amino-6-(2-hydroxyphenyl)pyridazin-4-yl)oxy)ethyl)benzyl)amino)-2-oxoethyl)-2,6-diazaspiro[3.3]heptane-2-carboxylate (37.00 mg, 0.064 mmol, 1.00 equiv) in DCM (1.20 mL) was added TFA (0.40 mL) dropwise at room temperature. The resulting mixture was stirred for 2 h at room temperature. The resulting mixture was concentrated under reduced pressure. The residue was purified by reverse flash chromatography with the following conditions: column, C18 silica gel; mobile phase, MeCN in water (0.1% FA), 10% to 50% gradient in 10 min; detector, UV 254 nm. to afford the title compound (26.80 mg, 87.86%) as a white solid. LCMS (ESI) m/z: [M+H]⁺=475.

The following intermediates in Table A15 were prepared in a similar manner as described in the preparation of K-65.

TABLE A15

| | | | LCMS |
|---|---|---|---|
| | Intermediate | | (ESI)m/z: |
| Structure | No. | Name | [M + H]⁺ |
| 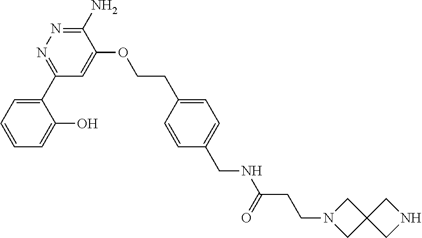 [SMILES: Nc1nnc(-c2ccccc2O)cc1OCCc1ccc(CNC(=O)CCN2CC3(CNC3)C2)cc1] | K-66 | N-(4-(2-((3-amino-6-(2- hydroxyphenyl)pyridazin-4- yl)oxy)ethyl)benzyl)-3-(2,6- diazaspiro[3.3]heptan-2- yl)propanamide | 488 |
| 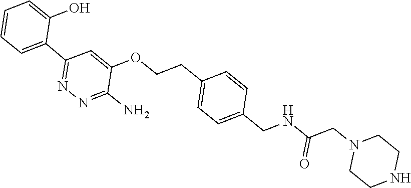 [SMILES: Nc1nnc(-c2ccccc2O)cc1OCCc1ccc(CNC(=O)CN2CCNCC2)cc1] | K-67 | N-(4-(2-((3-amino-6-(2- hydroxyphenyl)pyridazin-4- yl)oxy)ethyl)benzyl)-2-(piperazin-1- yl)acetamide | 463 |
| 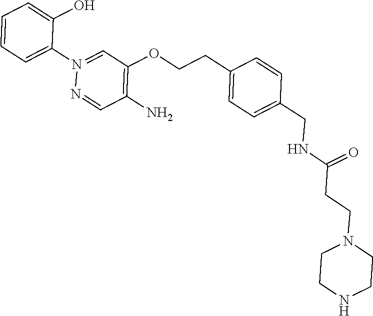 | K-68 | N-(4-(2-((3-amino-6-(2- hydroxyphenyl)pyridazin-4- yl)oxy)ethyl)benzyl)-3-(piperazin-1- yl)propanamide | 477 |
| 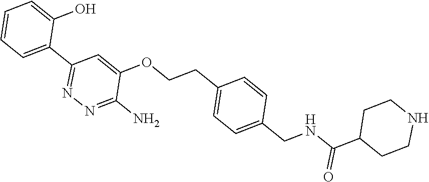 [SMILES: Nc1nnc(-c2ccccc2O)cc1OCCc1ccc(CNC(=O)C2CCNCC2)cc1] | K-69 | N-(4-(2-((3-amino-6-(2- hydroxyphenyl)pyridazin-4- yl)oxy)ethyl)benzyl)piperidine-4- carboxamide | 448 |
| 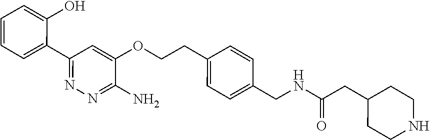 [SMILES: Nc1nnc(-c2ccccc2O)cc1OCCc1ccc(CNC(=O)CC2CCNCC2)cc1] | K-70 | N-(4-(2-((3-amino-6-(2- hydroxyphenyl)pyridazin-4- yl)oxy)ethyl)benzyl)-2-(piperidin-4- yl)acetamide | 462 |
| 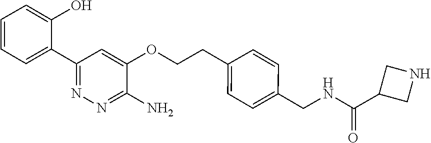 [SMILES: Nc1nnc(-c2ccccc2O)cc1OCCc1ccc(CNC(=O)C2CNC2)cc1] | K-71 | N-(4-(2-((3-amino-6-(2- hydroxyphenyl)pyridazin-4- yl)oxy)ethyl)benzyl)azetidine-3- carboxamide | 420 |
| 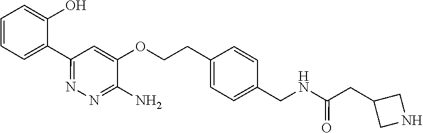 [SMILES: Nc1nnc(-c2ccccc2O)cc1OCCc1ccc(CNC(=O)CC2CNC2)cc1] | K-72 | N-(4-(2-((3-amino-6-(2- hydroxyphenyl)pyridazin-4- yl)oxy)ethyl)benzyl)-2-(azetidin-3- yl)acetamide | 434 |
| 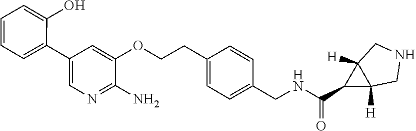 [SMILES: Nc1ncc(-c2ccccc2O)cc1OCCc1ccc(CNC(=O)[C@H]2[C@@H]3CNC[C@@H]32)cc1] | K-73 | (1R,5S,6r)-N-(4-(2-((3-amino-6-(2- hydroxyphenyl)pyridazin-4- yl)oxy)ethyl)benzyl)-3- azabicyclo[3.1.0]hexane-6- carboxamide | 446 |
| 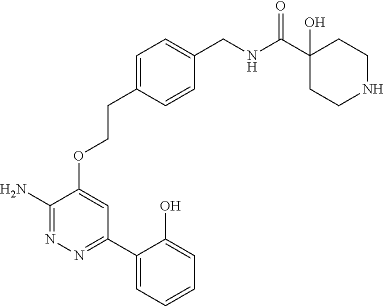 [SMILES: Nc1nnc(-c2ccccc2O)cc1OCCc1ccc(CNC(=O)C2(O)CCNCC2)cc1] | K-74 | N-[[4-(2-[[3-amino-6-(2- hydroxyphenyl)pyridazin-4- yl]oxy]ethyl)phenyl]methyl]-4- hydroxypiperidine-4-carboxamide | 464 |
| 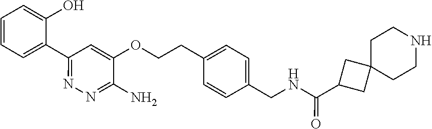 [SMILES: Nc1nnc(-c2ccccc2O)cc1OCCc1ccc(CNC(=O)C2CC3(CCNCC3)C2)cc1] | K-75 | N-(4-(2-((3-amino-6-(2- hydroxyphenyl)pyridazin-4- yl)oxy)ethyl)benzyl)-7- azaspiro[3.5]nonane-2- carboxamide | 487.60 |
| 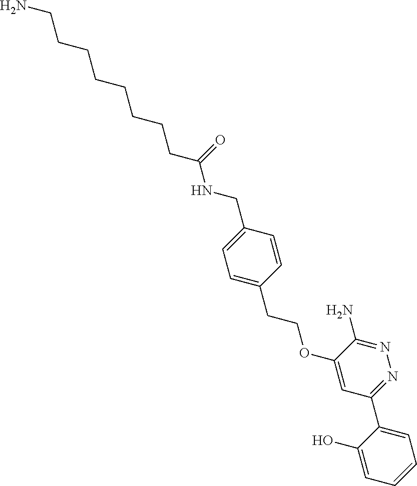 [SMILES: NCCCCCCCCC(=O)NCc1ccc(CCOc2cc(-c3ccccc3O)nnc2N)cc1] | K-76 | 9-amino-N-(4-(2-((3-amino-6-(2- hydroxyphenyl)pyridazin-4- yl)oxy)ethyl)benzyl)nonanamide | 491.64 |
| 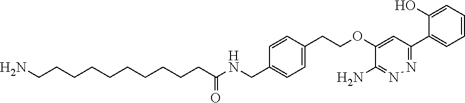 [SMILES: NCCCCCCCCCCC(=O)NCc1ccc(CCOc2cc(-c3ccccc3O)nnc2N)cc1] | K-77 | 11-amino-N-(4-(2-((3-amino-6-(2- hydroxyphenyl)pyridazin-4- yl)oxy)ethyl)benzyl)undecanamide | 520 |
| 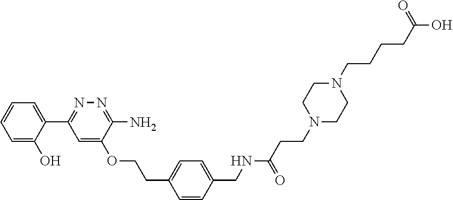 [SMILES: Nc1nnc(-c2ccccc2O)cc1OCCc1ccc(CNC(=O)CCN2CCN(CCCCC(=O)O)CC2)cc1] | K-78 | 5-(4-(3-((4-(2-((3-amino-6-(2- hydroxyphenyl)pyridazin-4- yl)oxy)ethyl)benzyl)amino)-3- oxopropyl)piperazin-1-yl) pentanoic acid | 685 |

### Preparation of 4-(2-[[3-amino-6-(2-hydroxyphenyl)pyridazin-4-yl](methyl)amino]ethyl)benzoic acid (I-66)

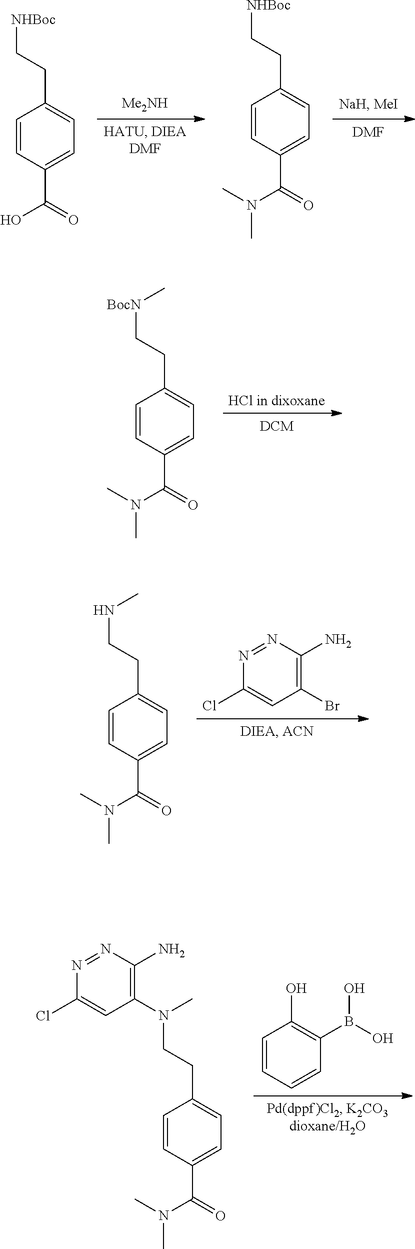

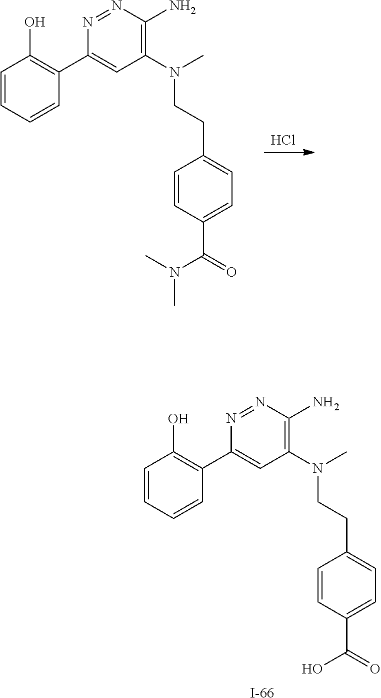

### Step 1: Preparation of tert-butyl N-[2-[4-(dimethylcarbamoyl)phenyl]ethyl]carbamate

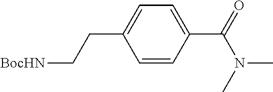 [SMILES: CN(C)C(=O)c1ccc(CCNC(=O)OC(C)(C)C)cc1]

To a stirred solution of 4-[2-[(tert-butoxycarbonyl)amino]ethyl]benzoic acid (5.0 g, 18.8 mmol, 1.0 equiv) in DMF (50.0 mL) were added DIEA (7.3 g, 56.5 mmol, 3.0 equiv) and HATU (1.3 equiv). The resulting mixture was stirred for 30 min at room temperature followed by addition of dimethylamine (1.02 g, 22.6 mmol, 1.2 equiv). The resulting mixture was stirred for an additional 2 h at room temperature, then diluted with water and extracted with EtOAc (3×15 mL). The combined organic layers were washed with brine (15 mL), dried over anhydrous Na₂SO₄, filtered, and concentrated under reduced pressure to afford tert-butyl N-[2-[4-(dimethylcarbamoyl)phenyl]ethyl]carbamate (5.9 g) as a yellow oil. The crude product was used in the next step without further purification. LCMS (ESI) m/z: [M+H]⁺=237.1.

### Step 2: Preparation of tert-butyl N-[2-[4-(dimethylcarbamoyl)phenyl]ethyl]-N-methylcarbamate

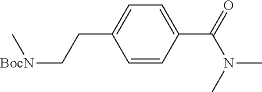

Into a 250 mL round-bottomed flask were added tert-butyl N-[2-[4-(dimethylcarbamoyl)phenyl]ethyl]carbamate (5.9 g, 20.2 mmol, 1.0 equiv) and DMF (100.0 mL). To the above mixture was added NaH (1.94 g, 80.8 mmol, 4.0 equiv) in portions at 0° C. The mixture was stirred for 30 min at room temperature, followed by dropwise addition of methyl iodide (5.7 g, 40.4 mmol, 2.0 equiv). The mixture was stirred for an additional 1 h and then quenched with water at 0° C. and extracted with EtOAc (3×30 mL). The combined organic layers were washed with brine (30 mL), dried over anhydrous Na₂SO₄, filtered, and concentrated under reduced pressure. The residue was purified by silica gel column chromatography (petroleum ether/EtOAc 1:1) to afford tert-butyl N-[2-[4-(dimethylcarbamoyl)phenyl]ethyl]-N-methylcarbamate (4 g, 64.69%) as a pale yellow oil. LCMS (ESI) m/z: [M+H]+=251.1

### Step 3: Preparation of N,N-dimethyl-4-[2-(methylamino)ethyl]benzamide

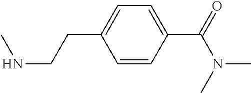

Into a 250 mL round-bottomed flask containing tert-butyl N-[2-[4-(dimethylcarbamoyl)phenyl]ethyl]-N-methylcarbamate (4.9 g, 16.0 mmol, 1.0 equiv) in DCM (50.0 mL) was added 4 M HCl in dioxane (50.0 mL) at 0° C. The resulting mixture was stirred for 2 h at room temperature and then concentrated under vacuum to give N,N-dimethyl-4-[2-(methylamino)ethyl]benzamide (4 g, quant.) as a yellow solid. The crude product was directly used in the next step without further purification. LCMS (ESI) m/z: [M+H]+=207.3

### Step 4: Preparation of 4-[2-[(3-amino-6-chloropyridazin-4-yl)(methyl)amino]ethyl]-N,N-dimethylbenzamide

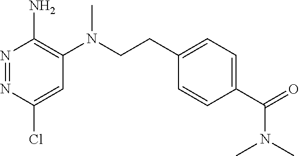 [SMILES: CN(C)C(=O)c1ccc(CCN(C)c2cc(Cl)nnc2N)cc1]

Into a 250 mL round-bottomed flask containing N,N-dimethyl-4-[2-(methylamino)ethyl]benzamide (4.0 g, 19.4 mmol, 1.0 equiv) in ACN (72.0 mL) were added DIEA (12.5 g, 96.9 mmol, 5.0 equiv) and 4-bromo-6-chloropyridazin-3-amine (4.9 g, 23.3 mmol, 1.2 equiv). The resulting mixture was stirred for 16 h at 100° C. under an atmosphere of dry nitrogen. The mixture was then cooled down to room temperature and concentrated under reduced pressure. The residue was purified by silica gel column chromatography (petroleum ether/EtOAc 1:1) to afford 4-[2-[(3-amino-6-chloropyridazin-4-yl)(methyl)amino]ethyl]-N,N-dimethylbenzamide (2.3 g, 35.53%) as a yellow solid. LCMS (ESI) m/z: [M+H]+=334.3

### Step 5: Preparation of 4-(2-[[3-amino-6-(2-hydroxyphenyl)pyridazin-4-yl](methyl)amino]ethyl)-N,N-dimethylbenzamide

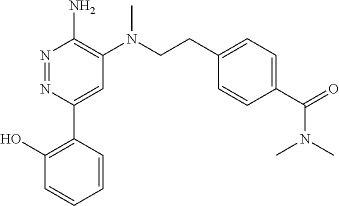 [SMILES: CN(C)C(=O)c1ccc(CCN(C)c2cc(-c3ccccc3O)nnc2N)cc1]

To a solution of 4-[2-[(3-amino-6-chloropyridazin-4-yl)(methyl)amino]ethyl]-N,N-dimethylbenzamide (1.0 g, 3.0 mmol, 1.0 equiv) and 2-hydroxyphenylboronic acid (537 mg, 3.9 mmol, 1.3 equiv) in dioxane (60.0 mL) and water (3.0 mL) was added K₂CO₃ (1.2 g, 9.0 mmol, 3.0 equiv) and Pd(dppf)Cl₂ (219 mg, 0.3 mmol, 0.1 equiv). After stirring for 2 h at 80° C. under a nitrogen atmosphere, the resulting mixture was cooled to room temperature and concentrated under reduced pressure. The residue was purified by silica gel column chromatography (petroleum ether/EtOAc 1:1) to afford 4-(2-[[3-amino-6-(2-hydroxyphenyl)pyridazin-4-yl](methyl)amino]ethyl)-N,N-dimethylbenzamide (530 mg, 45.19%) as a brown solid. LCMS (ESI) m/z: [M+H]⁺=392.2.

### Step 6: Preparation of 4-(2-[[3-amino-6-(2-hydroxyphenyl)pyridazin-4-yl](methyl)amino]ethyl)benzoic acid (I-66)

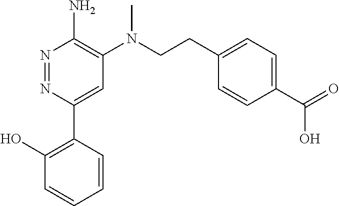 [SMILES: CN(CCc1ccc(C(=O)O)cc1)c1cc(-c2ccccc2O)nnc1N]

To a 25 mL round-bottomed flask were added 4-(2-[[3-amino-6-(2-hydroxyphenyl)pyridazin-4-yl](methyl)amino]ethyl)-N,N-dimethylbenzamide (360 mg, 0.9 mmol, 1.0 equiv) and 12 N HCl (5.0 mL). The resulting mixture was stirred for 16 h at 70° C., then cooled to room temperature and concentrated under reduced pressure. The crude product was purified by reversed-phase preparative HPLC to afford 4-(2-[[3-amino-6-(2-hydroxyphenyl)pyridazin-4-yl](methyl)amino]ethyl)benzoic acid (I-66, 37 mg, 10.26%) as a grey solid. LCMS (ESI) m/z: [M+H]⁺=365.3.

### Preparation of 4-(2-((3-amino-6-(2-hydroxyphenyl)pyridazin-4-yl)(ethyl)amino)ethyl)benzoic acid (K-79)

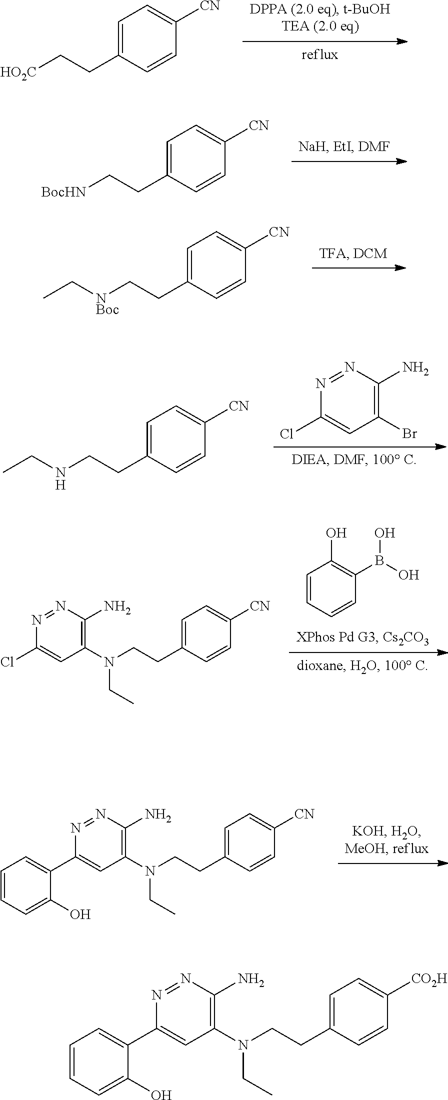

### Step 1: Preparation of tert-butyl N-[2-(4-cyanophenyl)ethyl]carbamate

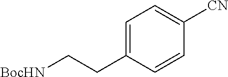 [SMILES: CC(C)(C)OC(=O)NCCc1ccc(C#N)cc1]

To a stirred solution of 3-(4-cyanophenyl)propanoic acid (2.00 g, 11.416 mmol, 1.00 equiv) in t-BuOH (20.00 mL) was added DPPA (6.28 g, 22.820 mmol, 2.00 equiv) and TEA (2.31 g, 22.833 mmol, 2.00 equiv) at room temperature. The resulting mixture was stirred for overnight at 60 degrees C. The resulting mixture was concentrated under reduced pressure, then washed with citric acid, followed by brine. The organic phase was separated and dried over anhydrous sodium sulfate, filtered and concentrated to give a crude product. The crude product was purified by silica gel column chromatography, eluting with PE/EtOAc (4:1) to afford tert-butyl N-[2-(4-cyanophenyl)ethyl]carbamate (1.5 g, 50.68%) as a white solid. LCMS (ESI) m/z [M+H]⁺=247.

### Step 2: Preparation of tert-butyl N-[2-(4-cyanophenyl)ethyl]-N-ethylcarbamate

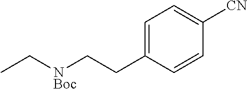 [SMILES: CCN(CCc1ccc(C#N)cc1)C(=O)OC(C)(C)C]

To a stirred solution of tert-butyl N-[2-(4-cyanophenyl)ethyl]carbamate (1.50 g, 6.090 mmol, 1.00 equiv) in DMF (20.00 mL) was added ethyl iodide (1.14 g, 7.309 mmol, 1.20 equiv) and NaH (0.49 g, 12.251 mmol, 2.01 equiv, 60%) at 0 degrees C. The resulting mixture was stirred for 2 h at room temperature. The reaction was quenched by the addition of ice water (100 mL) at room temperature. The resulting mixture was extracted with EtOAc (3×100 mL). The combined organic layers were washed with brine (3×100 mL) and dried over anhydrous Na₂SO₄. After filtration, the filtrate was concentrated under reduced pressure to afford tert-butyl N-[2-(4-cyanophenyl)ethyl]-N-ethylcarbamate (1.1 g, 60.57%) as a white solid that was used in the next step directly without further purification. LCMS (ESI) m/z: [M+H]⁺=275.

### Step 3: Preparation of 4-[2-(ethylamino)ethyl]benzonitrile

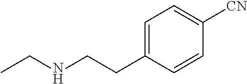

To a stirred solution of tert-butyl N-[2-(4-cyanophenyl)ethyl]-N-ethylcarbamate (1.10 g, 4.009 mmol, 1.00 equiv) in DCM (10.00 mL) was added TFA (3.67 mL, 49.409 mmol, 12.32 equiv) at room temperature. After stirring for 2 h at room temperature, the resulting mixture was concentrated under reduced pressure. The residue was purified by reverse flash chromatography with the following conditions: column, C18 silica gel; mobile phase, MeCN in water (0.2% FA), 0% to 100% gradient in 30 min; detector, UV 254 nm. The product was collected at gradient 10% to afford 4-[2-(ethylamino)ethyl]benzonitrile (600 mg, 77.30%) as a colorless syrup. LCMS (ESI) m/z [M+H]⁺=175.

### Step 4: Preparation of 4-[2-[(3-amino-6-chloropyridazin-4-yl)(ethyl)amino]ethyl]benzonitrile

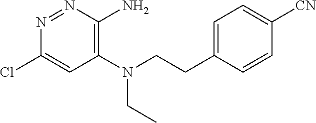 [SMILES: CCN(CCc1ccc(C#N)cc1)c1cc(Cl)nnc1N]

To a stirred mixture of 4-[2-(ethylamino)ethyl]benzonitrile (600.00 mg, 3.443 mmol, 1.00 equiv) and 4-bromo-6-chloropyridazin-3-amine (1435.48 mg, 6.886 mmol, 2.00 equiv) in DMF (10.00 mL) was added DIEA (1335.10 mg, 10.329 mmol, 3.00 equiv) at room temperature. The resulting mixture was stirred for overnight at 100 degrees C. The mixture was diluted with water (100 mL) and extracted with EtOAc (3×100 mL). The combined organic layers were washed with brine (3×100 mL), then dried over anhydrous Na₂SO₄. After filtration, the filtrate was concentrated under reduced pressure. The residue was purified by silica gel column chromatography, eluting with PE/EtOAc (3:1) to afford 4-[2-[(3-amino-6-chloropyridazin-4-yl)(ethyl)amino]ethyl]benzonitrile (360 mg, 31.87%) as a yellow solid. LCMS (ESI) m/z [M+H]⁺=302.

### Step 5: Preparation of 4-(2-[[3-amino-6-(2-hydroxyphenyl)pyridazin-4-yl](ethyl)amino]ethyl) benzonitrile

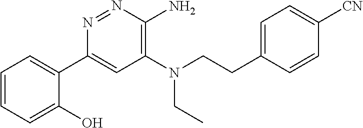 [SMILES: CCN(CCc1ccc(C#N)cc1)c1cc(-c2ccccc2O)nnc1N]

To a solution of 4-[2-[(3-amino-6-chloropyridazin-4-yl)(ethyl)amino]ethyl]benzonitrile (360.00 mg, 1.193 mmol, 1.00 equiv) and 2-hydroxyphenylboronic acid (493.62 mg, 3.579 mmol, 3.00 equiv) in dioxane (5.00 mL) and H₂O (1.00 mL) were added Cs₂CO₃ (1166.03 mg, 3.579 mmol, 3.00 equiv) and XPhos Pd G3 (100.97 mg, 0.119 mmol, 0.10 equiv). After stirring for 4 h at 95 degrees C. under a nitrogen atmosphere. The mixture was allowed to cool to room temperature. The reaction mixture was filtered and concentrated in vacuo. The residue was purified by reverse flash chromatography with the following conditions: column, C18 silica gel; mobile phase, ACN in water (0.2% FA), 0% to 100% gradient in 10 min; detector, UV 254 nm. The product was collected at gradient 30% to afford 4-(2-[[3-amino-6-(2-hydroxyphenyl)pyridazin-4-yl](ethyl)amino]ethyl) benzonitrile (320 mg, 67.17%) as a yellow solid. LCMS (ESI) m/z [M+H]⁺=360.

### Step 6: Preparation of 4-(2-[[3-amino-6-(2-hydroxyphenyl)pyridazin-4-yl](ethyl)amino]ethyl) benzoic acid (K-79)

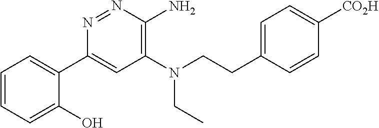 [SMILES: CCN(CCc1ccc(C(=O)O)cc1)c1cc(-c2ccccc2O)nnc1N]

To a stirred mixture of 4-(2-[[3-amino-6-(2-hydroxyphenyl)pyridazin-4-yl](ethyl)amino]ethyl) benzonitrile (120.00 mg, 0.334 mmol, 1.00 equiv) in H₂O (1.00 mL) and MeOH (1.00 mL) was added KOH (32.41 mg, 0.578 mmol, 1.73 equiv) in portions. The resulting mixture was stirred for 2 h at 80 degrees C. The mixture was allowed to cool to room temperature. The reaction mixture was filtered through a short pad of Celite and concentrated in vacuo. The residue was purified by reverse flash chromatography with the following conditions: column, C18 silica gel; mobile phase, ACN in water (0.2% FA), 0% to 100% gradient in 30 min; detector, UV 254 nm. This provided 4-(2-[[3-amino-6-(2-hydroxyphenyl)pyridazin-4-yl](ethyl)amino]ethyl) benzoic acid (61 mg, 45.87%) as a white solid. LCMS (ESI) m/z [M+H]⁺=379.

### Preparation of 2-[6-amino-5-[4-(2-aminoethyl)piperazin-1-yl]pyridazin-3-yl]phenol (I-67)

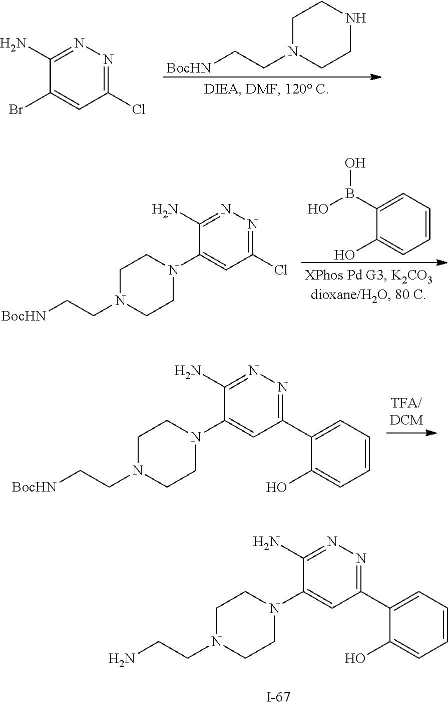

### Step 1: Preparation of tert-butyl N-[2-[4-(3-amino-6-chloropyridazin-4-yl)piperazin-1-yl]ethyl]carbamate

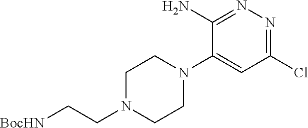 [SMILES: CC(C)(C)OC(=O)NCCN1CCN(c2cc(Cl)nnc2N)CC1]

To a solution of 3-amino-4-bromo-6-chloropyridazine (4.16 g, 19.958 mmol, 1.00 equiv) in DMF (25.00 mL) were added tert-butyl N-[2-(piperazin-1-yl)ethyl]carbamate (9.15 g, 0.040 mmol, 2 equiv) and DIEA (9.90 mL, 56.837 mmol, 2.85 equiv). The mixture was stirred overnight at 120° C. After cooling to room temperature, the solvent was removed under vacuum. The residue was purified by silica gel column chromatography, eluting with DCM/MeOH (20:1) to afford tert-butyl N-[2-[4-(3-amino-6-chloropyridazin-4-yl)piperazin-1-yl]ethyl]carbamate (2.1329 g, 29.95%) as a yellow solid. LCMS (ESI) m/z: [M+H]⁺=357.

### Step 2: Preparation of tert-butyl N-(2-[4-[3-amino-6-(2-hydroxyphenyl)pyridazin-4-yl]piperazin-1-yl]ethyl)carbamate

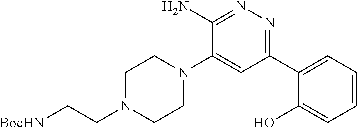 [SMILES: CC(C)(C)OC(=O)NCCN1CCN(c2cc(-c3ccccc3O)nnc2N)CC1]

To a solution of tert-butyl N-[2-[4-(3-amino-6-chloropyridazin-4-yl)piperazin-1-yl]ethyl]carbamate (1.98 g, 5.548 mmol, 1.00 equiv) in dioxane/water (4:1, 50 mL) were added (2-hydroxyphenyl)boronic acid (1.15 g, 8.338 mmol, 1.50 equiv), K₂CO₃ (3 eq, 2.3 g), and XPhos Pd G3 (0.1 eq, 0.555 mmol, 469.64 mg). The resulting mixture was stirred at 80° C. under an atmosphere of dry nitrogen. After cooling to room temperature, the solvent was removed under vacuum. The residue was applied onto a silica gel column, eluted with DCM/MeOH (30:1) to afford tert-butyl N-(2-[4-[3-amino-6-(2-hydroxyphenyl)pyridazin-4-yl]piperazin-1-yl]ethyl)carbamate (1.325 g, 57.6%) as a yellow solid. LCMS (ESI) m/z: [M+H]⁺=415.

### Step 3: Preparation of 2-[6-amino-5-[4-(2-aminoethyl)piperazin-1-yl]pyridazin-3-yl]phenol (I-67)

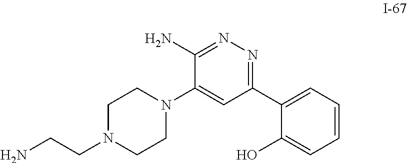 [SMILES: NCCN1CCN(c2cc(-c3ccccc3O)nnc2N)CC1]

A solution of tert-butyl N-(2-[4-[3-amino-6-(2-hydroxyphenyl)pyridazin-4-yl]piperazin-1-yl]ethyl)carbamate (1.3 g, 3.316 mmol, 1.00 equiv) in TFA/DCM (1:5, 108 mL) was stirred overnight at room temperature. After removing the solvent under vacuum, the residue was purified by reversed phase flash chromatography. This provided I-67 (0.497 g, 50.4%) as a yellow solid that was used without further purification. LCMS (ESI) m/z: [M+H]⁺=315.

### Preparation of 2-(6-amino-5-(4-((methylamino)methyl)phenethoxy)pyridazin-3-yl)phenol (I-68)

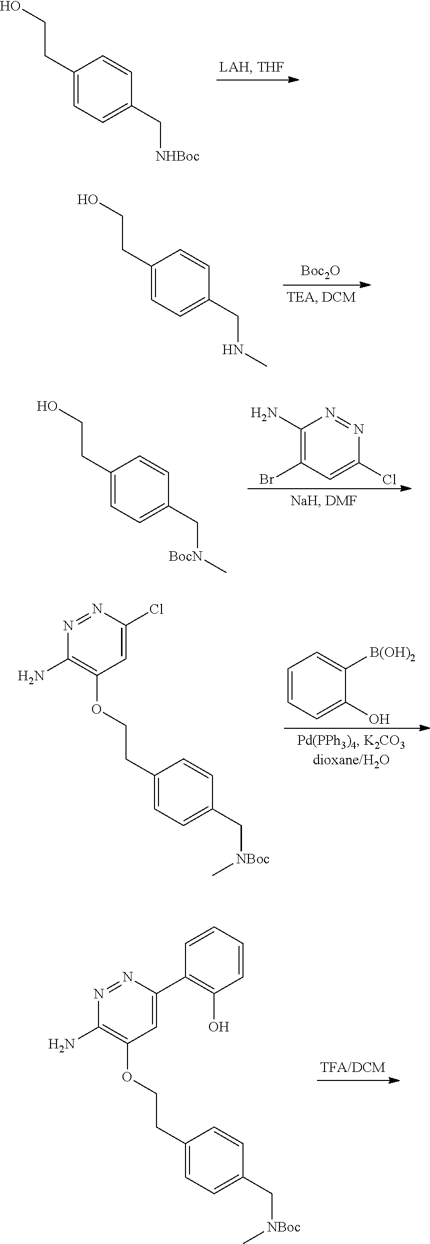

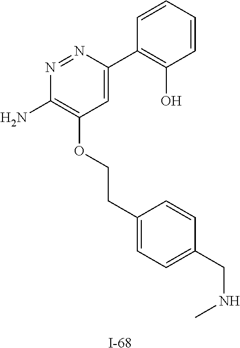

### Step 1: Preparation of 2-(4-((methylamino)methyl)phenyl)ethan-1-ol

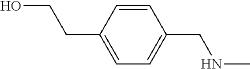

To a stirred solution of tert-butyl N-[[4-(2-hydroxyethyl)phenyl]methyl]carbamate (1.00 g, 3.979 mmol, 1.00 equiv) in THF (40 mL) was added LiAlH₄ (906.09 mg, 23.873 mmol, 6.00 equiv) in portions at 0° C. under an atmosphere of dry nitrogen. The resulting mixture was stirred for 4 h at 80° C., then cooled to room temperature, diluted with DCM (30 mL), and quenched with water. The resulting mixture was concentrated under reduced pressure and the residue purified by reversed phase flash chromatography to afford 2-(4-((methylamino)methyl)phenyl)ethan-1-ol (1.3 g) as an off-white solid. LCMS (ESI) m/z [M+H]⁺=166.

### Step 2: Preparation of tert-butyl (4-(2-hydroxyethyl)benzyl)(methyl)carbamate

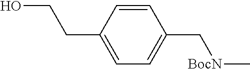 [SMILES: CN(Cc1ccc(CCO)cc1)C(=O)OC(C)(C)C]

To a stirred mixture of 2-(4-((methylamino)methyl)phenyl)ethan-1-ol (800.00 mg, 4.842 mmol, 1.00 equiv) and TEA (979.83 mg, 9.683 mmol, 2.00 equiv) in DCM (50 mL) was added di-tert-butyl dicarbonate (1585.00 mg, 7.262 mmol, 1.50 equiv) dropwise at room temperature under an atmosphere of dry nitrogen. The resulting mixture was stirred overnight, then concentrated under reduced pressure. The residue was purified by reversed phase flash chromatography to afford tert-butyl (4-(2-hydroxyethyl)benzyl)(methyl)carbamate (380 mg, 22.18%) as a white solid. LCMS (ESI) m/z [M+H]⁺=266.

### Step 3: Preparation of tert-butyl (4-(2-((3-amino-6-chloropyridazin-4-yl)oxy)ethyl)benzyl)(methyl) carbamate

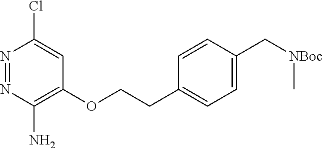 [SMILES: CN(Cc1ccc(CCOc2cc(Cl)nnc2N)cc1)C(=O)OC(C)(C)C]

To a stirred mixture of 4-bromo-6-chloropyridazin-3-amine (226.86 mg, 1.088 mmol, 0.76 equiv) and tert-butyl (4-(2-hydroxyethyl)benzyl)(methyl)carbamate (380.00 mg, 1.432 mmol, 1.00 equiv) in DMF (10 mL) was added NaH (51.89 mg, 2.162 mmol, 1.51 equiv) in portions at room temperature under an atmosphere of dry nitrogen. The resulting mixture was stirred for 3 h at 50° C. then cooled to room temperature and quenched with water (20 mL). The mixture was diluted with EtOAc (10 mL) and washed with brine (2×10 mL). The organic layer was dried over Na₂SO₄, filtered, and concentrated under reduced pressure. The residue was purified by reversed phase flash chromatography to afford tert-butyl (4-(2-((3-amino-6-chloropyridazin-4-yl)oxy)ethyl)benzyl)(methyl)carbamate (150 mg, 26.66%) as a brown solid. LCMS (ESI) m/z [M+H]⁺=393.

### Step 4: Preparation of tert-butyl (4-(2-((3-amino-6-(2-hydroxyphenyl)pyridazin-4-yl)oxy)ethyl)benzyl) (methyl)carbamate

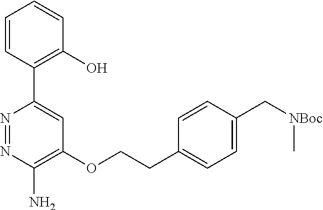 [SMILES: CN(Cc1ccc(CCOc2cc(-c3ccccc3O)nnc2N)cc1)C(=O)OC(C)(C)C]

To a solution of tert-butyl (4-(2-((3-amino-6-chloropyridazin-4-yl)oxy)ethyl)benzyl)(methyl) carbamate (100.00 mg, 0.255 mmol, 1.00 equiv) and 2-hydroxyphenylboronic acid (52.66 mg, 0.382 mmol, 1.50 equiv) in dioxane (2 mL) and water (0.5 mL) were added K₂CO₃ (70.36 mg, 0.509 mmol, 2.00 equiv) and Pd(PPh₃)₄ (29.41 mg, 0.025 mmol, 0.10 equiv). After stirring for 4 h at 100° C. under an atmosphere of dry nitrogen, the resulting mixture was cooled to room temperature and concentrated under reduced pressure. The residue was purified by Preparative TLC (DCM/MeOH 10:1) to afford tert-butyl (4-(2-((3-amino-6-(2-hydroxyphenyl)pyridazin-4-yl)oxy)ethyl)benzyl)(methyl)carbamate (140 mg) as a brown solid. LCMS (ESI) m/z [M+H]⁺=451.

### Step 5: Preparation of 2-(6-amino-5-(4-((methylamino)methyl)phenethoxy)pyridazin-3-yl)phenol (I-68)

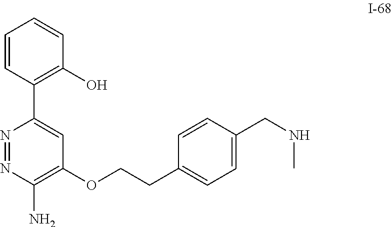 [SMILES: CNCc1ccc(CCOc2cc(-c3ccccc3O)nnc2N)cc1]

To a stirred solution of tert-butyl (4-(2-((3-amino-6-(2-hydroxyphenyl)pyridazin-4-yl)oxy)ethyl)benzyl)(methyl)carbamate (140.00 mg, 0.311 mmol, 1.00 equiv) in DCM (3.0 mL) was added TFA (1.00 mL) dropwise at room temperature under an atmosphere of dry nitrogen. The resulting mixture was stirred for 1 h, then concentrated under reduced pressure. The residue was purified by reversed phase flash chromatography to afford I-68 (80 mg, 73.50%) as a brown oil. LCMS (ESI) m/z [M+H]+=351.

### Preparation of 4-(2-((3-amino-6-(2-hydroxyphenyl)pyridazin-4-yl)(methyl)amino)ethyl) benzaldehyde (K-80)

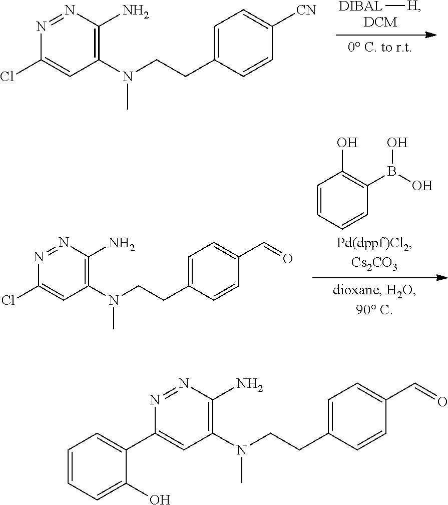

### Step 1: Preparation of 4-(2-((3-amino-6-chloropyridazin-4-yl)(methyl)amino)ethyl)benzaldehyde

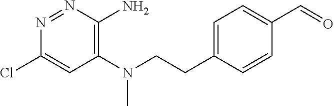 [SMILES: CN(CCc1ccc(C=O)cc1)c1cc(Cl)nnc1N]

To a stirred mixture of 4-[2-[(3-amino-6-chloropyridazin-4-yl)(methyl)amino]ethyl] benzonitrile (200.00 mg, 0.695 mmol, 1.00 equiv) in anhydrous DCM (3.0 mL) was added DIBAL-H (0.97 mL, 1.5 M in toluene, 1.459 mmol, 2.10 equiv) dropwise at 0 degrees C. under a nitrogen atmosphere. The resulting mixture was stirred for 1 h at room temperature. The mixture was acidified to pH 3 with HCl (0.5 M) at 0 degrees C., then neutralized to pH 7 with saturated NaHCO₃ (aq.). The resulting mixture was extracted with DCM (3×100 mL). The combined organic layers were washed with brine (3×100 mL) and dried over anhydrous Na₂SO₄. After filtration, the filtrate was concentrated under reduced pressure to result in 4-(2-((3-amino-6-chloropyridazin-4-yl)(methyl)amino)ethyl)benzaldehyde (162 mg, 75.84%) as a white solid. This material was used directly in the next step without further purification. LCMS (ESI) m/z: [M+H]⁺=291.

### Step 2: Preparation of 4-(2-((3-amino-6-(2-hydroxyphenyl)pyridazin-4-yl)(methyl)amino)ethyl) benzaldehyde (K-80)

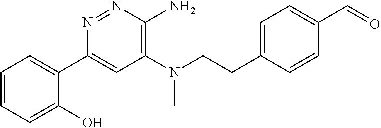 [SMILES: CN(CCc1ccc(C=O)cc1)c1cc(-c2ccccc2O)nnc1N]

To a stirred mixture of 4-(2-((3-amino-6-chloropyridazin-4-yl)(methyl)amino)ethyl)benzaldehyde (237.20 mg, 1.720 mmol, 5.00 equiv) in dioxane (5.00 mL) and H₂O (1.00 mL) was added Pd(dtbpf)Cl₂ (22.42 mg, 0.034 mmol, 0.10 equiv) and Cs₂CO₃ (448.25 mg, 1.376 mmol, 4.00 equiv). The resulting mixture was stirred overnight at 100 degrees C. under nitrogen atmosphere. The mixture was allowed to cool to room temperature, then filtered through a short pad of Celite and concentrated in vacuo. The residue was purified by reverse flash chromatography under the following conditions (column, C18 silica gel; mobile phase, ACN in water (0.1% FA), 0% to 60% gradient in 40 min; detector, UV 254 nm). This provided 4-(2-((3-amino-6-(2-hydroxyphenyl)pyridazin-4-yl)(methyl)amino)ethyl) benzaldehyde (29 mg, 20.57%) as a yellow solid. LCMS (ESI) m/z [M+H]+=349.

### Preparation of 4-(2-((3-amino-6-(2-hydroxyphenyl)pyridazin-4-yl)amino)ethyl)benzaldehyde (K-81)

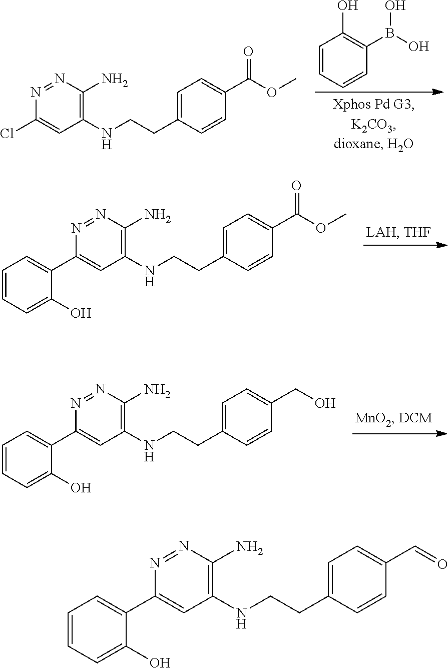

### Step 1: Preparation of methyl 4-(2-((3-amino-6-(2-hydroxyphenyl)pyridazin-4-yl)amino)ethyl)benzoate

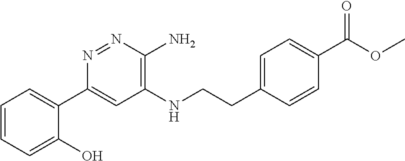 [SMILES: COC(=O)c1ccc(CCNc2cc(-c3ccccc3O)nnc2N)cc1]

Into a 40-mL vial purged and maintained with an inert atmosphere of nitrogen was placed methyl 4-[2-[(3-amino-6-chloropyridazin-4-yl)amino]ethyl]benzoate (250.00 mg, 0.815 mmol, 1.00 equiv), 2-hydroxyphenylboronic acid (168.62 mg, 1.222 mmol, 1.5 equiv), Xphos Pd G3 (68.99 mg, 0.081 mmol, 0.1 equiv), K₂CO₃ (225.27 mg, 1.630 mmol, 2 equiv), H₂O (1.00 mL) and 1,4-dioxane (10.00 mL). The resulting mixture was stirred for 12 h at 80 degrees C. The mixture was diluted with water (30 mL), extracted with 2×50 mL of ethyl acetate and the organic layers combined and dried over anhydrous sodium sulfate, and concentrated under reduced pressure. The crude product was purified by Flash-Prep-HPLC with the following conditions (IntelFlash-1): Column, silica gel; mobile phase, ACN=0 increasing to ACN=30 within 40 min; Detector: 254 nm. This provided 200 mg (67.34%) of methyl 4-(2-((3-amino-6-(2-hydroxyphenyl)pyridazin-4-yl)amino)ethyl)benzoate as a yellow solid. LCMS (ESI) m/z: [M+H]⁺=365.

### Step 2: Preparation of 2-(6-amino-5-((4-(hydroxymethyl)phenethyl)amino)pyridazin-3-yl)phenol

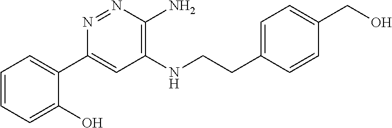 [SMILES: Nc1nnc(-c2ccccc2O)cc1NCCc1ccc(CO)cc1]

Into a 8-mL sealed tube was placed methyl 4-(2-((3-amino-6-(2-hydroxyphenyl)pyridazin-4-yl)amino)ethyl)benzoate (150.00 mg, 0.412 mmol, 1.00 equiv) and THF (2.00 mL), and the vessel was cooled to 0 degrees C. LAH (31.25 mg, 0.823 mmol, 2 equiv) was added at 0 degrees C. The resulting solution was stirred for 1 h at 0 degrees C. The reaction was quenched by the addition of 0.3 mL of water and 0.9 mL of 10% aqueous NaOH. The solids were removed by filtration and filtrate was concentrated under vacuum. This resulted in 110 mg (crude) of 2-(6-amino-5-((4-(hydroxymethyl)phenethyl)amino)pyridazin-3-yl)phenol as an off-white solid. LCMS (ESI) m/z: [M+H]⁺=337.

### Step 3: Preparation of 4-(2-((3-amino-6-(2-hydroxyphenyl)pyridazin-4-yl)amino)ethyl)benzaldehyde (K-81)

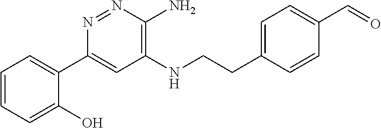 [SMILES: Nc1nnc(-c2ccccc2O)cc1NCCc1ccc(C=O)cc1]

Into a 50-mL round-bottom flask, was placed 2-(6-amino-5-((4-(hydroxymethyl)phenethyl)amino)pyridazin-3-yl)pheno (90.00 mg, 0.268 mmol, 1.00 equiv), DCM (20.00 mL), and MnO₂ (465.19 mg, 5.351 mmol, 20 equiv). The resulting solution was stirred for 24 h at room temperature. The solids were filtered off. The filtrate was concentrated under vacuum and the residue was purified by silica gel column with ethyl acetate/petroleum ether (4:1). This resulted in 25 mg (27.95%) of 4-(2-[[3-amino-6-(2-hydroxyphenyl)pyridazin-4-yl]amino]ethyl)benzaldehyde as a dark yellow solid. LCMS (ESI) m/z: [M+H]⁺=335.

### Preparation of 4-(2-((3-amino-6-(2-hydroxyphenyl)pyridazin-4-yl)amino)ethyl)benzoic acid (K-82)

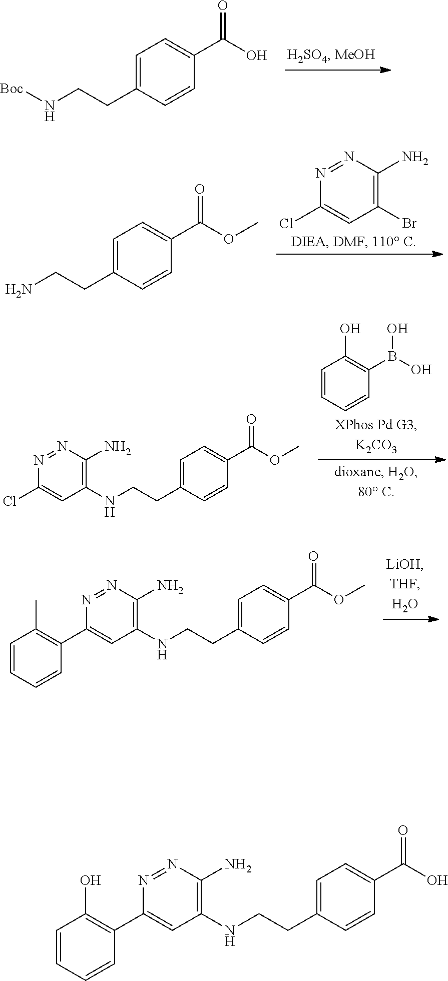

### Step 1: Preparation of methyl 4-(2-aminoethyl)benzoate

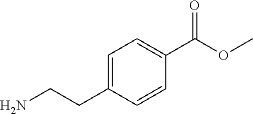

To a solution of 4-[2-[(tert-butoxycarbonyl)amino]ethyl]benzoic acid (3.00 g, 11.308 mmol, 1.00 equiv) in MeOH (20.00 mL) was added H₂SO₄ (2.50 mL, 46.901 mmol, 4.15 equiv), and the resulting solution was stirred at 25 degrees C. for 20 hours. The resulting mixture was concentrated under reduced pressure and diluted with water (20 mL). The mixture was adjusted pH 7-8 with saturated sodium bicarbonate solution and extracted with CH₂Cl₂ (50 mL×3). The organic layers were combined and dried over anhydrous sodium sulfate, filtered, and concentrated to give methyl 4-(2-aminoethyl)benzoate (1.4 g, 69.08%) as a brown solid that was used in the next step directly without further purification. LCMS (ESI) m/z: [M+H]⁺=180.

### Step 2: Preparation of methyl 4-[2-[(3-amino-6-chloropyridazin-4-yl)amino]ethyl]benzoate

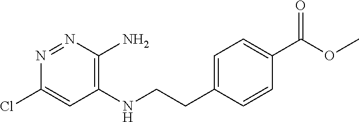 [SMILES: COC(=O)c1ccc(CCNc2cc(Cl)nnc2N)cc1]

To a solution of methyl 4-(2-aminoethyl)benzoate (1.40 g, 7.812 mmol, 1.00 equiv) and 4-bromo-6-chloropyridazin-3-amine (1.95 g, 9.374 mmol, 1.2 equiv) in DMF (10 mL) was added DIEA (2.02 g, 15.623 mmol, 2 equiv). The resulting solution was stirred at 110 degrees C. for 15 hours. The mixture was allowed to cool to room temperature, then filtered through a short pad of Celite and filtrate concentrated in vacuo. The residue was purified by flash C18 chromatography, elution gradient 0 to 26% ACN in H₂O, to give methyl 4-[2-[(3-amino-6-chloropyridazin-4-yl)amino]ethyl]benzoate (305 mg, 12.73%) as a yellow solid. LCMS (ESI) m/z: [M+H]⁺=307.

### Step 3: Preparation of methyl 4-(2-[[3-amino-6-(2-hydroxyphenyl)pyridazin-4-yl]amino]ethyl)benzoate

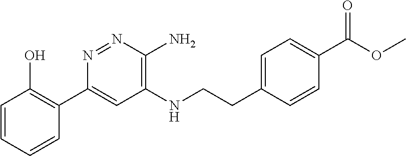 [SMILES: COC(=O)c1ccc(CCNc2cc(-c3ccccc3O)nnc2N)cc1]

To a solution of methyl 4-[2-[(3-amino-6-chloropyridazin-4-yl)amino]ethyl]benzoate and 2-hydroxyphenylboronic acid (70.0 mg, 0.652 mmol, 2 equiv) in dioxane (4.00 mL) and H₂O (1.00 mL) were added Xphos Pd G3 (27.6 mg, 0.033 mmol, 0.1 equiv) and K₂CO₃ (90.1, 0.652 mmol, 2 equiv). The resulting solution was stirred at 80 degrees C. for 3 hours. The mixture was diluted with water (30 mL) and extracted with EtOAc (30 mL×3). The organic layers were combined and dried over anhydrous sodium sulfate, filtered and concentrated to give a crude product. The crude product was purified by Prep-TLC (CH₂Cl₂/MeOH 10:1) to give methyl 4-(2-[[3-amino-6-(2-hydroxyphenyl)pyridazin-4-yl]amino]ethyl)benzoate (75 mg, 63.13%) as a yellow solid. LCMS (ESI) m/z: [M+H]⁺=365.

### Step 4: Preparation of 4-(2-[[3-amino-6-(2-hydroxyphenyl)pyridazin-4-yl]amino]ethyl)benzoic acid (K-82)

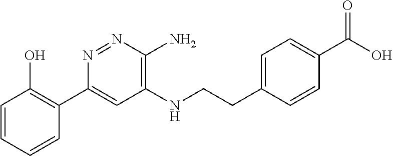 [SMILES: Nc1nnc(-c2ccccc2O)cc1NCCc1ccc(C(=O)O)cc1]

To a solution of methyl 4-(2-[[3-amino-6-(2-hydroxyphenyl)pyridazin-4-yl]amino]ethyl)benzoate (55.0 mg, 0.151 mmol, 1.00 equiv) in THF (0.50 mL) and H₂O (0.10 mL) was added LiOH (36.2 mg, 1.509 mmol, 10 equiv), and the resulting solution was stirred at 25 degrees C. for 15 hours. The mixture was diluted with water (10 mL) and extracted with EtOAc (10 mL×3). The organic layers were combined, dried over anhydrous sodium sulfate, filtered, and concentrated. The crude product was purified by flash C18 chromatography, elution gradient 0 to 26% ACN in H₂O to give 4-(2-[[3-amino-6-(2-hydroxyphenyl)pyridazin-4-yl]amino]ethyl)benzoic acid (32 mg, 60.51%) as a yellow solid. LCMS (ESI) m/z: [M+H]⁺=351.

### Preparation of 2-(6-amino-5-((4-(aminomethyl)phenethyl)amino)pyridazin-3-yl)phenol (K-83)

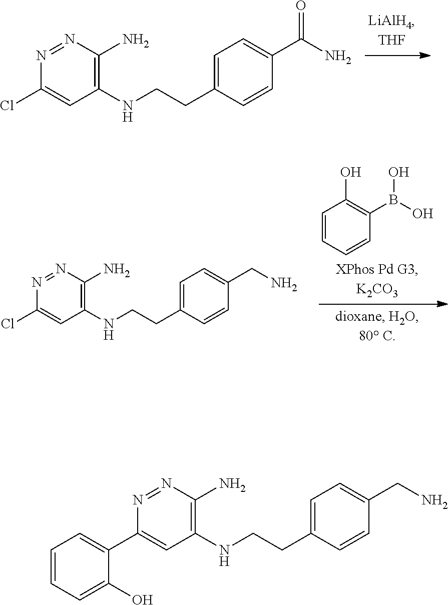 [SMILES: NC(=O)c1ccc(CCNc2cc(Cl)nnc2N)cc1 ; OB(O)c1ccccc1O ; NCc1ccc(CCNc2cc(Cl)nnc2N)cc1 ; NCc1ccc(CCNc2cc(-c3ccccc3O)nnc2N)cc1]

### Step 1: Preparation of N4-[2-[4-(aminomethyl)phenyl]ethyl]-6-chloropyridazine-3,4-diamine

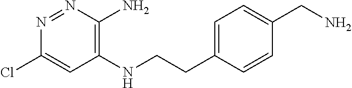 [SMILES: NCc1ccc(CCNc2cc(Cl)nnc2N)cc1]

To a solution of 4-[2-[(3-amino-6-chloropyridazin-4-yl)amino]ethyl]benzamide (100.00 mg, 0.343 mmol, 1.00 equiv) in THF (3 mL) was added LiAlH₄ (130.10 mg, 3.428 mmol, 10 equiv). The resulting solution was stirred at 25 degrees C. for 5 hours. The mixture was quenched carefully with water (5 mL), diluted with EtOAc (50 mL), and washed with water (50 mL×3). The organic layer was dried over anhydrous sodium sulfate, filtered, and concentrated to give a crude product. The crude product was purified by flash C18 chromatography, elution gradient 0 to 22% ACN in H₂O to give N4-[2-[4-(aminomethyl)phenyl]ethyl]-6-chloropyridazine-3,4-diamine (37 mg, 38.86%) as a white solid. LCMS (ESI) m/z: [M+H]⁺=278.

### Step 2: Preparation of 2-[6-amino-5-([2-[4-(aminomethyl)phenyl]ethyl]amino)pyridazin-3-yl]phenol (K-83)

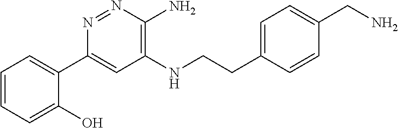 [SMILES: NCc1ccc(CCNc2cc(-c3ccccc3O)nnc2N)cc1]

To a stirred solution of N4-[2-[4-(aminomethyl)phenyl]ethyl]-6-chloropyridazine-3,4-diamine (37.00 mg, 0.133 mmol, 1.00 equiv) and 2-hydroxyphenylboronic acid (36.75 mg, 0.266 mmol, 2 equiv) in dioxane (1.6 mL) and H₂O (0.4 mL) was added Xphos Pd G3 (11.28 mg, 0.013 mmol, 0.1 equiv) and K₂CO₃ (36.82 mg, 0.266 mmol, 2.00 equiv). The resulting mixture was stirred for 2 h at 80 degrees C. The mixture was allowed to cool to room temperature. The reaction mixture was filtered through a short pad of Celite and concentrated in vacuo. The residue was purified by flash C18 chromatography, elution gradient 0 to 8% ACN in H₂O to give 2-[6-amino-5-([2-[4-(aminomethyl)phenyl]ethyl]amino)pyridazin-3-yl]phenol (19 mg, 42.52%) as a brown solid. LCMS (ESI) m/z: [M+H]⁺=336.

### Preparation of 4-(4-(2-((4-(3-(3-amino-6-(2-hydroxyphenyl)pyridazin-4-yl)-3,8-diazabicyclo[3.2.1]octan-8-yl)pyridin-2-yl)oxy)ethyl)piperazin-1-yl)butanoic acid (K-84)

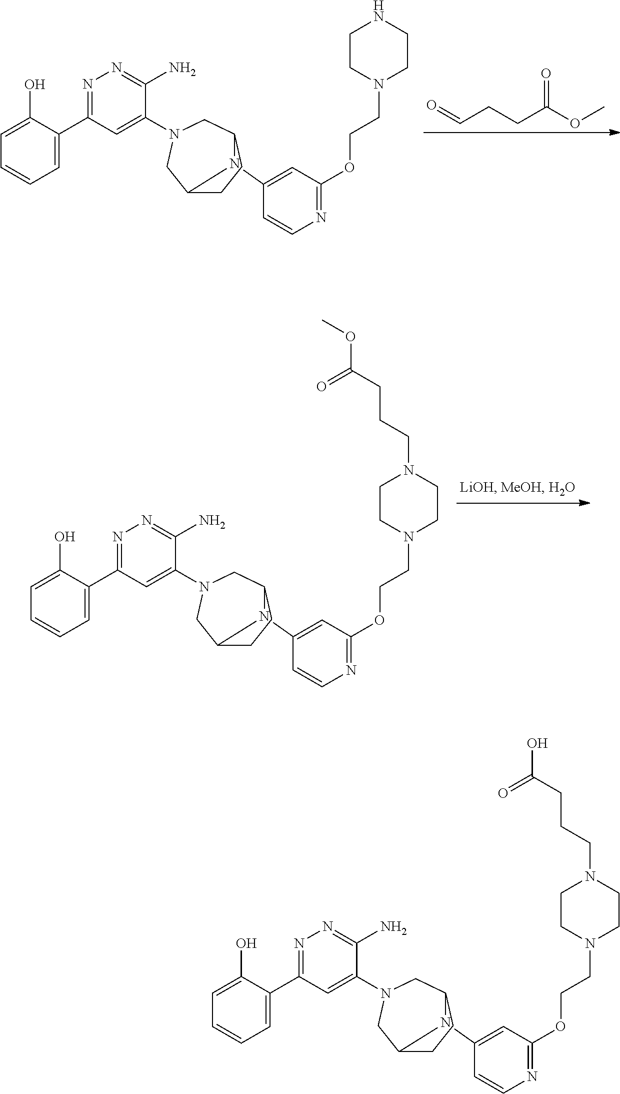

### Step 1: Preparation of methyl 5-(4-[2-[(4-[3-[3-amino-6-(2-hydroxyphenyl)pyridazin-4-yl]-3,8-diazabicyclo[3.2.1]octan-8-yl]pyridin-2-yl)oxy]ethyl]piperazin-1-yl)pentanoate

To a stirred solution of 2-[6-amino-5-(8-[2-[2-(piperazin-1-yl)ethoxy]pyridin-4-yl]-3,8-diazabicyclo[3.2.1]octan-3-yl)pyridazin-3-yl]phenol (20 mg, 0.040 mmol, 1.00 equiv) and methyl 4-oxobutanoate (9.24 mg, 2.00 equiv) in MeOH (2.00 mL) was added AcOH (0.03 mL, 0,040 mmol) and NaBH₃CN (12.50 mg, 0.200 mmol, 5.00 equiv) at room temperature. The resulting mixture was stirred for 2 h. The reaction was quenched with H2O at room temperature. The residue was purified by reverse flash chromatography to afford the title compound (8.9 mg, 36.26%). LCMS (ESI) m/z: [M+H]⁺=603.

### Step 2: Preparation of 4-(4-(2-((4-(3-(3-amino-6-(2-hydroxyphenyl)pyridazin-4-yl)-3,8-diazabicyclo[3.2.1]octan-8-yl)pyridin-2-yl)oxy)ethyl)piperazin-1-yl)butanoic acid (K-84)

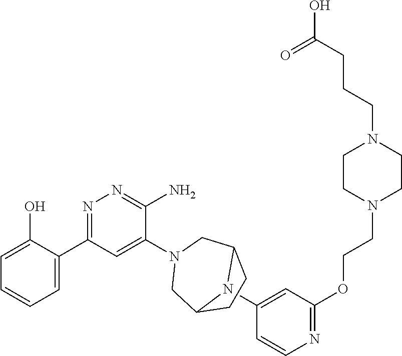 [SMILES: Nc1nnc(-c2ccccc2O)cc1N1CC2CCC(C1)N2c1ccnc(OCCN2CCN(CCCC(=O)O)CC2)c1]

To a stirred mixture of methyl 5-(4-[2-[(4-[3-[3-amino-6-(2-hydroxyphenyl)pyridazin-4-yl]-3,8-diazabicyclo[3.2.1]octan-8-yl]pyridin-2-yl)oxy]ethyl]piperazin-1-yl)pentanoate (8.00 mg, 0.013 mmol, 1.00 equiv) in MeOH (0.60 mL) and H₂O (0.30 mL) was added LiOH (3.18 mg, 0.130 mmol, 10.00 equiv) at room temperature. After 4 h the mixture was neutralized to pH 7 with HCl (1 M). The resulting mixture was concentrated under reduced pressure to afford the title compound which was used in the next step directly without further purification. LCMS (ESI) m/z: [M+H]⁺=589.

The following intermediates in Table A16 were prepared in a similar manner as described in the preparation of K-84.

TABLE A16

| | | | LCMS |
|---|---|---|---|
| | Intermediate | | (ESI)m/z: |
| Structure | No. | Name | [M + H]⁺ |
| 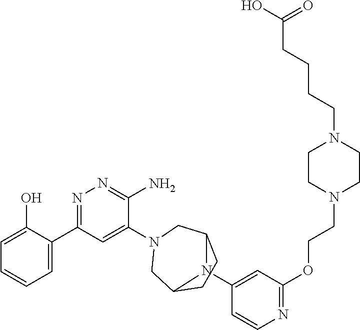 [SMILES: Nc1nnc(-c2ccccc2O)cc1N1CC2CCC(C1)N2c1ccnc(OCCN2CCN(CCCCC(=O)O)CC2)c1] | K-85 | N-(4-(2-((3-amino-6-(2- hydroxyphenyl)pyridazin-4- yl)oxy)ethyl)benzyl)-3-(2,6- diazaspiro[3.3]heptan-2- yl)propanamide | 603.0 |

### Preparation of 2-(6-amino-5-(8-(2-((1r,3r)-3-(piperidin-4-yloxy)cyclobutoxy)pyridin-4-yl)-3,8-diazabicyclo[3.2.1]octan-3-yl)pyridazin-3-yl)phenol (K-86)

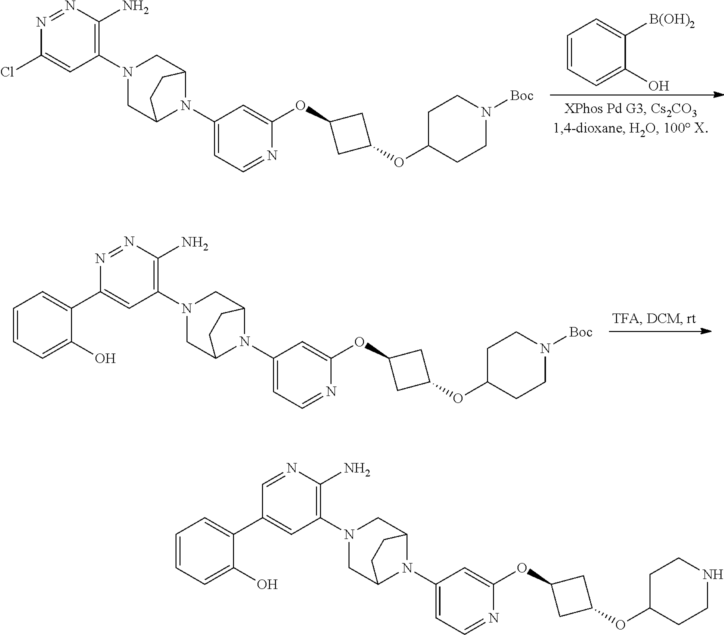 [SMILES: CC(C)(C)OC(=O)N1CCC(O[C@H]2C[C@H](Oc3cc(N4C5CCC4CN(c4cc(Cl)nnc4N)C5)ccn3)C2)CC1 ; OB(O)c1ccccc1O ; CC(C)(C)OC(=O)N1CCC(O[C@H]2C[C@H](Oc3cc(N4C5CCC4CN(c4cc(-c6ccccc6O)nnc4N)C5)ccn3)C2)CC1 ; Nc1ncc(-c2ccccc2O)cc1N1CC2CCC(C1)N2c1ccnc(O[C@H]2C[C@H](OC3CCNCC3)C2)c1]

### Step 1: Preparation of tert-butyl 4-((1r,3r)-3-((4-(3-(3-amino-6-(2-hydroxyphenyl)pyridazin-4-yl)-3,8-diazabicyclo[3.2.1]octan-8-yl)pyridin-2-yl)oxy)cyclobutoxy)piperidine-1-carboxylate

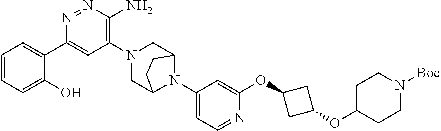 [SMILES: CC(C)(C)OC(=O)N1CCC(O[C@H]2C[C@H](Oc3cc(N4C5CCC4CN(c4cc(-c6ccccc6O)nnc4N)C5)ccn3)C2)CC1]

To a stirred solution of tert-butyl 4-[(1r,3r)-3-([4-[3-(3-amino-6-chloropyridazin-4-yl)-3,8-diazabicyclo[3.2.1]octan-8-yl]pyridin-2-yl]oxy)cyclobutoxy]piperidine-1-carboxylate (200.00 mg, 0.341 mmol, 1.00 equiv) and 2-hydroxyphenylboronic acid (141.19 mg, 1.023 mmol, 3.00 equiv) in 1,4-dioxane (8.00 mL) and H₂O (2.00 mL) was added XPhos Pd G3 (57.77 mg, 0.068 mmol, 0.20 equiv) and Cs₂CO₃ (333.53 mg, 1.023 mmol, 3.00 equiv) at room temperature. The resulting mixture was stirred for 1 h at 100 degrees C. under nitrogen atmosphere. The mixture was allowed to cool to room temperature. The residue was purified by reverse flash chromatography to provide the title compound (224 mg, crude) as a brown solid. LCMS (ESI) m/z: [M+H]⁺=644.

### Step 2: Preparation of 2-[6-amino-5-(8-[2-[(1r,3r)-3-(piperidin-4-yloxy)cyclobutoxy]pyridin-4-yl]-3,8-diazabicyclo[3.2.1]octan-3-yl)pyridazin-3-yl]phenol (K-86)

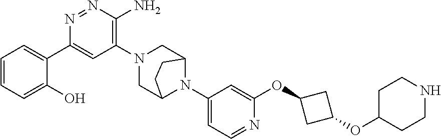 [SMILES: Nc1nnc(-c2ccccc2O)cc1N1CC2CCC(C1)N2c1ccnc(O[C@H]2C[C@H](OC3CCNCC3)C2)c1]

To a stirred solution of tert-butyl 4-((1 r,3r)-3-((4-(3-(3-amino-6-(2-hydroxyphenyl)pyridazin-4-yl)-3,8-diazabicyclo[3.2.1]octan-8-yl)pyridin-2-yl)oxy)cyclobutoxy)piperidine-1-carboxylate (224.00 mg, 0.348 mmol, 1.00 equiv) in DCM (6.00 mL) was added TFA (3.00 mL, 40.389 mmol, 116.08 equiv) at room temperature. The resulting mixture was stirred for 1 h at room temperature, then concentrated under reduced pressure, to provide the title compound (373 mg, crude) as a brown oil. LCMS (ESI) m/z: [M+H]⁺=544.

### Preparation of 2-(6-amino-5-(4-(piperazin-1-ylmethyl)phenethoxy)pyridazin-3-yl)phenol (K-87)

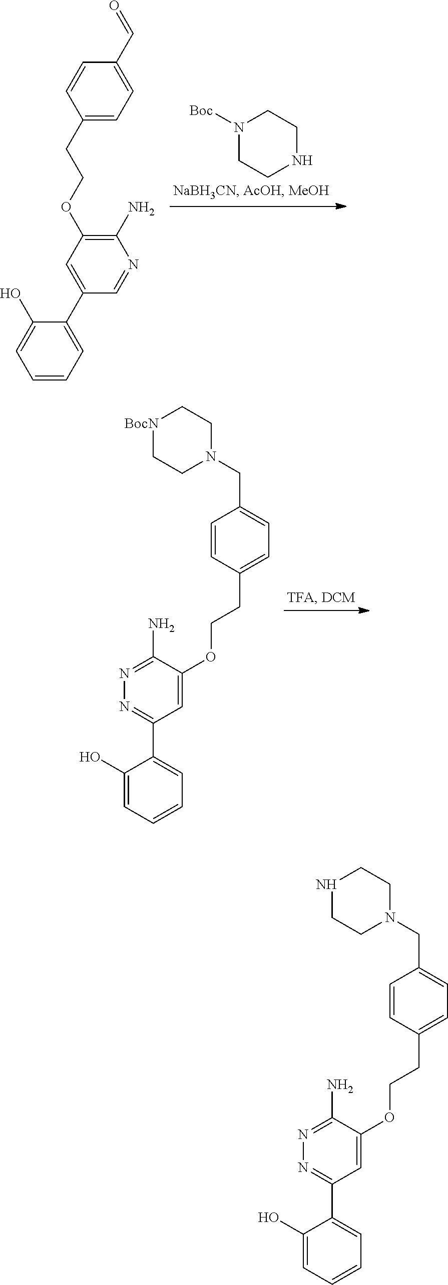 [SMILES: Nc1ncc(-c2ccccc2O)cc1OCCc1ccc(C=O)cc1 ; CC(C)(C)OC(=O)N1CCNCC1 ; CC(C)(C)OC(=O)N1CCN(Cc2ccc(CCOc3cc(-c4ccccc4O)nnc3N)cc2)CC1 ; Nc1nnc(-c2ccccc2O)cc1OCCc1ccc(CN2CCNCC2)cc1]

### Step 1: Preparation of tert-butyl 4-(4-(2-((3-amino-6-(2-hydroxyphenyl)pyridazin-4-yl)oxy)ethyl)benzyl)piperazine-1-carboxylate

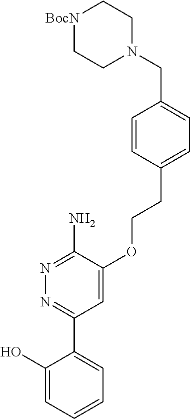 [SMILES: CC(C)(C)OC(=O)N1CCN(Cc2ccc(CCOc3cc(-c4ccccc4O)nnc3N)cc2)CC1]

To a stirred solution of 4-(2-[[3-amino-6-(2-hydroxyphenyl)pyridazin-4-yl]oxy]ethyl)benzaldehyde (80.00 mg, 0.24 mmol, 1.00 equiv) and tert-butyl piperazine-1-carboxylate (66.72 mg, 0.0368 mmol, 1.50 equiv) in a mixture of CH₂Cl₂ (4 mL) and methanol (4 mL) was added acetic acid until the solution reached pH 6. NaBH₃CN (60.00 mg, 0.960 mmol, 4.00 equiv) was then added in portions at room temperature. The resulting mixture was stirred for 2 h, then concentrated under reduced pressure. The residue was purified by reverse flash chromatography to afford the title compound (60 mg, 53.27%) as a white solid.

LCMS (ESI) m/z: [M+H]⁺=506.

### Step 2: Preparation of 2-(6-amino-5-(4-(piperazin-1-ylmethyl)phenethoxy)pyridazin-3-yl)phenol (K-87)

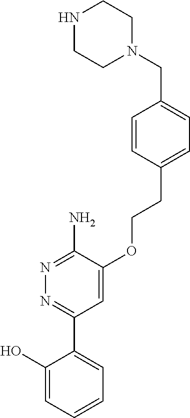 [SMILES: Nc1nnc(-c2ccccc2O)cc1OCCc1ccc(CN2CCNCC2)cc1]

To a stirred solution of tert-butyl 4-(4-(2-((3-amino-6-(2-hydroxyphenyl)pyridazin-4-yl)oxy)ethyl)benzyl)piperazine-1-carboxylate (60.00 mg, 0.12 mmol, 1.00 equiv) in DCM (6.00 mL) was added TFA (2.00 mL) dropwise at room temperature. The resulting mixture was stirred for 2 h, then concentrated in vacuo. The crude product mixture was used in the next step directly without further purification. This resulted in the title compound (68.00 mg, crude) as a brown yellow oil. LCMS (ESI) m/z: [M+H]⁺=405.

The following intermediates in Table A17 were prepared in a similar manner as described in the preparation of K-87.

TABLE A17

| | | | LCMS |
|---|---|---|---|
| | Intermediate | | (ESI)m/z: |
| Structure | No. | Name | [M + H]⁺ |
| 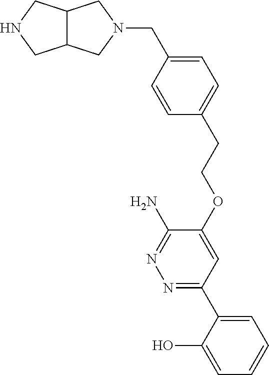 [SMILES: Nc1nnc(-c2ccccc2O)cc1OCCc1ccc(CN2CC3CNCC3C2)cc1] | K-88 | 2-(6-amino-5-(4- ((hexahydropyrrolo[3,4-c]pyrrol- 2(1H)- yl)methyl)phenethoxy)pyridazin-3- yl)phenol | 431.2 |

### Example 2. Preparation of N-(2-[[2-(2-[4-[3-amino-6-(2-hydroxyphenyl)pyridazin-4-yl]piperazin-1-yl]acetamido)ethyl](methyl)amino]ethyl)-2-[[2-(2,6-dioxopiperidin-3-yl)-1,3-dioxo-2,3-dihydro-1H-isoindol-4-yl]oxy]acetamide (compound 104)

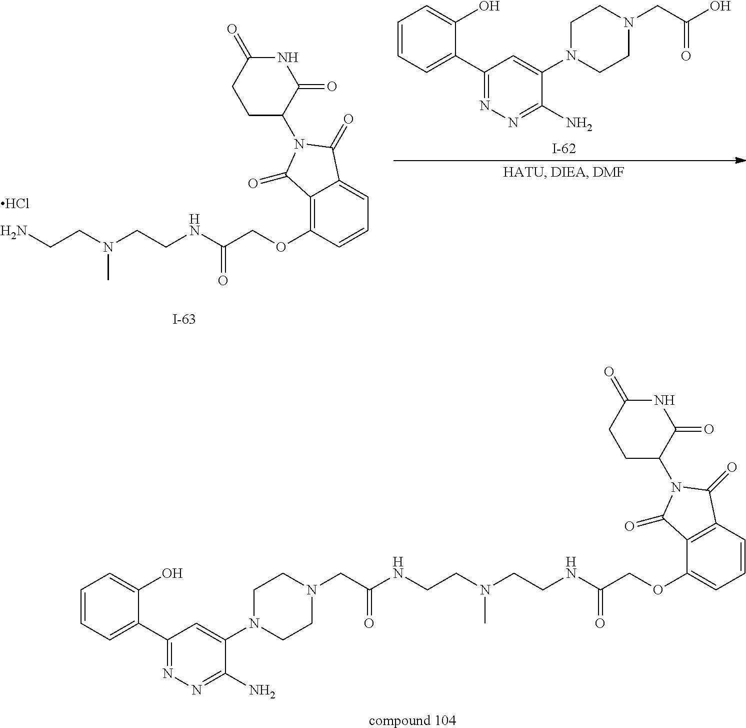

To a stirred mixture of 2-(4-(3-amino-6-(2-hydroxyphenyl)pyridazin-4-yl)piperazin-1-yl)acetic acid (I-62, 36.2 mg, 0.083 mmol, 1.20 equiv) in DMF (1 mL) was added DIEA (49.05 mg, 0.380 mmol, 5.00 equiv) and HATU (37.52 mg, 0.099 mmol, 1.10 equiv) at room temperature under an atmosphere of dry nitrogen and the resulting mixture was stirred for 30 minutes. To the above mixture was added N-(2-((2-aminoethyl) (methyl)amino)ethyl)-2-((2-(2,6-dioxopiperidin-3-yl)-1,3-dioxoisoindolin-4-yl)oxy)acetamide (I-63, 36.02 mg, 0.083 mmol, 1.10 equiv) portion wise. The resulting mixture was stirred for additional 1 h at room temperature. The reaction was quenched with water (5 mL) at room temperature and the resulting mixture was extracted with EtOAc (3×5 mL). The combined organic layers were washed with brine (10 mL), and dried over anhydrous Na₂SO₄. After filtration, the filtrate was concentrated under reduced pressure. The crude product was purified by reversed-phase preparative HPLC to afford compound 104 (4.7 mg, 8.00%) as a yellow solid. ¹H NMR (300 MHz, DMSO-d₆) δ 11.12 (s, 1H), 8.14 (s, 1H), 7.97 (d, J=5.9 Hz, 1H), 7.89 (d, J=8.0 Hz, 1H), 7.78 (t, J=7.8 Hz, 2H), 7.48 (d, J=6.6 Hz, 2H), 7.38 (d, J=8.6 Hz, 1H), 7.24 (t, J=7.8 Hz, 1H), 6.89 (d, J=7.7 Hz, 2H), 6.25 (s, 2H), 5.12 (dd, J=12.9, 5.3 Hz, 1H), 4.78 (s, 2H), 3.14 (s, 7H), 3.00-2.90 (m, 5H), 2.62-2.50 (m, 9H), 2.30 (s, 3H), 2.04 (dd, J=12.7, 6.5 Hz, 1H). LCMS (ESI) m/z [M+H]⁺=743.55.

The following compounds in Table B were prepared using procedures similar to those used for the preparation of compound 104.

TABLE B

| | | LCMS | |
|---|---|---|---|
| | | (ESI) | |
| No. | Name | m/z | ¹H NMR |
| 76 | 4-[[1-(2-[4-[3-amino-6-(2- | 655.4 | 1HNMR (300 MHz, DMSO-d6) δ 14.20 (brs, |
| | hydroxyphenyl)pyridazin-4- | | 1H), 11.11 (s, 1H), 7.98-7.69 (m, 2H), 7.65- |
| | yl]piperazin-1-yl]acetyl)azetidin- | | 7.43 (m, 3H), 7.24 (td, J = 7.6, 1.5 Hz, 1H), |
| | 3-yl]methoxy]-2-(2,6- | | 6.95-6.83 (m, 2H), 6.26 (brs, 2H), 5.09 (dd, |
| | dioxopiperidin-3-yl)isoindole-1,3- | | J = 12.8, 5.4 Hz, 1H), 4.38 (t, J = 7.0 Hz, 3H), |
| | dione | | 4.17 (dd, J = 9.1, 5.5 Hz, 1H), 4.02 (t, J = 9.1 |
| | | | Hz, 1H), 3.80 (dd, J = 9.7, 5.5 Hz, 1H), 3.11 |
| | | | (s, 7H), 2.97-2.74 (m, 2H), 2.61 (s, 4H), 2.51 |
| | | | (q, J = 1.8 Hz, 1H), 2.13-1.95 (m, 1H) |
| 79 | 2-[4-[3-amino-6-(2- | 720.15 | ¹H NMR (300 MHz, DMSO-d6) δ 14.19 (brs, |
| | hydroxyphenyl)pyridazin-4- | | 1H), 11.11 (s, 1H), 8.08 (t, J = 5.8 Hz, 1H), |
| | yl]piperazin-1-yl]-N-[2-(2-[[2-(2,6- | | 7.91 (dd, J = 8.4, 1.6 Hz, 1H), 7.62 (dd, J = |
| | dioxopiperidin-3-yl)-1,3- | | 8.5, 7.1 Hz, 1H), 7.48 (s, 1H), 7.25 (td, J = |
| | dioxoisoindol-4- | | 7.6, 1.5 Hz, 1H), 7.18 (d, J = 8.6 Hz, 1H), 7.08 |
| | yl]amino]ethane- | | (d, J = 7.0 Hz, 1H), 6.97-6.82 (m, 3H), 6.27 |
| | sulfonyl)ethyl]acetamide | | (s, 2H), 5.05 (dd, J = 12.8, 5.4 Hz, 1H), 3.79 |
| | | | (q, J = 6.6 Hz, 2H), 3.54 (dt, J = 19.3, 6.4 Hz, |
| | | | 5H), 3.15 (s, 1H), 3.10-3.04 (m, 4H), 3.02 (s, |
| | | | 2H), 2.97-2.77 (m, 1H), 2.68 (d, J = 4.9 Hz, |
| | | | 4H), 2.60 (d, J = 3.0 Hz, 2H), 2.14-1.89 (m, |
| | | | 1H) |
| 80 | 2-[4-[3-amino-6-(2- | 762.35 | ¹H NMR (300 MHz, DMSO-d6) δ 14.13 (brs, |
| | hydroxyphenyl)pyridazin-4- | | 1H), 11.11 (s, 1H), 8.01-7.79 (m, 2H), 7.58 |
| | yl]piperazin-1-yl]-N-[4-(3-[[2-(2,6- | | (dd, J = 8.6, 7.1 Hz, 1H), 7.50 (s, 1H), 7.24 |
| | dioxopiperidin-3-yl)-1,3- | | (td, J = 7.6, 1.5 Hz, 1H), 7.14 (d, J = 8.6 Hz, |
| | dioxoisoindol-4- | | 1H), 7.03 (d, J = 7.0 Hz, 1H), 6.97-6.83 (m, |
| | yl]amino]propane- | | 2H), 6.76 (t, J = 6.3 Hz, 1H), 6.29 (s, 2H), |
| | sulfonyl)butyl]acetamide | | 5.06 (dd, J = 12.8, 5.4 Hz, 1H), 3.46 (q, J = |
| | | | 6.8 Hz, 2H), 3.25-3.10 (m, 10H), 3.10-3.02 |
| | | | (m, 2H), 2.99-2.80 (m, 1H), 2.79-2.65 (m, |
| | | | 3H), 2.66-2.54 (m, 1H), 2.08-1.88 (m, 3H), |
| | | | 1.76-1.58 (m, 4H) |
| 81 | 5-((1-(2-(4-(3-amino-6-(2- | 655.15 | ¹H NMR (300 MHz, DMSO-d6) δ 14.14 (brs, |
| | hydroxyphenyl)pyridazin-4- | | 1H), 11.12 (s, 1H), 7.92 (dd, J = 8.3, 1.6 Hz, |
| | yl)piperazin-1-yl)acetyl)azetidin- | | 1H), 7.86 (d, J = 8.3 Hz, 1H), 7.53 (s, 1H), |
| | 3-yl)methoxy)-2-(2,6- | | 7.49 (d, J = 2.3 Hz, 1H), 7.39 (dd, J = 8.3, 2.3 |
| | dioxopiperidin-3-yl)isoindoline- | | Hz, 1H), 7.25-7.16 (m, 1H), 7.00-6.76 (m, |
| | 1,3-dione | | 2H), 6.31 (s, 2H), 5.13 (dd, J = 12.9, 5.4 Hz, |
| | | | 1H), 4.38-4.30 (m, 3H), 4.21-3.90 (m, 2H), |
| | | | 3.76 (dd, J = 9.8, 5.3 Hz, 1H), 3.18 (s, 6H), |
| | | | 2.99-2.66 (m, 5H), 2.66-2.55 (m, 2H), |
| | | | 2.08-1.98 (m, 2H) |
| 82 | 2-[4-[3-amino-6-(2- | 712.4 | ¹H NMR (300 MHz, DMSO-d6) δ 11.14 (s, |
| | hydroxyphenyl)pyridazin-4- | | 1H), 10.03 (brs, 1H), 8.77 (brs, 1H), 7.92 (d, |
| | yl]piperazin-1-yl]-N-[1-(2-[[2-(2,6- | | J = 8.3 Hz, 1H), 7.75 (d, J = 7.8 Hz, 1H), 7.66- |
| | dioxopiperidin-3-yl)-1,3- | | 7.52 (m, 2H), 7.43 (dd, J = 8.4, 2.3 Hz, 1H), |
| | dioxoisoindol-5- | | 7.38 (t, J = 8.0 Hz, 1H), 7.14 (brs, 2H), 7.06- |
| | yl]oxy]ethyl)piperidin-4- | | 6.91 (m, 2H), 5.14 (dd, J = 12.9, 5.4 Hz, 1H), |
| | yl]acetamide | | 4.58 (t, J = 4.6 Hz, 2H), 3.94 (s, 4H), 3.62 (d, |
| | | | J = 28.8 Hz, 7H), 3.12 (m, 3H), 2.91 (ddd, J = |
| | | | 18.2, 13.8, 5.4 Hz, 1H), 2.67-2.54 (m, 2H), |
| | | | 2.14-1.94 (m, 3H), 1.73 (d, J = 12.7 Hz, 2H) |
| 83 | 2-[4-[3-amino-6-(2- | 712.45 | ¹H NMR (300 MHz, Methanol-d4) δ 7.76 (dd, |
| | hydroxyphenyl)pyridazin-4- | | J = 8.3, 1.6 Hz, 1H), 7.59-7.46 (m, 2H), 7.29- |
| | yl]piperazin-1-yl]-N-(8-[[2-(2,6- | | 7.26 (m, 1H), 6.97-6.93 (m, 3H), 6.80 (dd, |
| | dioxopiperidin-3-yl)-1,3- | | J = 8.4, 2.2 Hz, 1H), 5.07-5.01 (m, 1H), 3.31- |
| | dioxoisoindol-5- | | 3.09 (m, 11H), 2.93-2.61 (m, 7H), 2.21- |
| | yl]amino]octyl)acetamide | | 1.99 (m, 1H), 1.78-1.16 (m, 14H) |
| 86 | (2S,4R)-1-[(2S)-2-[2-(2-[2-[2-(2- | 931.50 | ¹H NMR (300 MHz, Methanol-d4) δ 8.88 (d, |
| | [4-[3-amino-6-(2- | | J = 2.5 Hz, 1H), 7.76 (d, J = 7.8 Hz, 1H), 7.65 |
| | hydroxyphenyl)pyridazin-4- | | (d, J = 9.5 Hz, 1H), 7.54 (d, J = 2.6 Hz, 1H), |
| | yl]piperazin-1- | | 7.52-7.36 (m, 4H), 7.31 (t, J = 8.1 Hz, 1H), |
| | yl]acetamido)ethoxy]eth- | | 6.97 (dd, J = 8.0, 3.7 Hz, 2H), 4.71 (s, 1H), |
| | oxy]ethoxy)acetamido]-3,3- | | 4.63-4.48 (m, 3H), 4.36 (dd, J = 15.6, 2.6 |
| | dimethylbutanoyl]-4-hydroxy-N- | | Hz, 1H), 4.03 (s, 2H), 3.85 (d, J = 12.9 Hz, |
| | [[4-(4-methyl-1,3-thiazol-5- | | 2H), 3.75-3.63 (m, 9H), 3.62-3.53 (m, 2H), |
| | yl)phenyl]methyl]pyrrolidine-2- | | 3.44 (p, J = 4.0, 3.5 Hz, 2H), 3.30 (s, 3H), |
| | carboxamide | | 3.18 (d, J = 2.8 Hz, 2H), 2.82 (d, J = 5.6 Hz, |
| | | | 4H), 2.53-2.42 (m, 3H), 2.25 (t, J = 10.7 Hz, |
| | | | 1H), 2.09 (td, J = 13.2, 11.6, 4.2 Hz, 1H), 1.05 |
| | | | (d, J = 2.6 Hz, 9H) |
| 87 | 2-[4-[3-amino-6-(2- | 670.45 | ¹H NMR (300 MHz, Methanol-d₄) δ 7.77 (d, |
| | hydroxyphenyl)pyridazin-4- | | J = 8.3 Hz, 1H), 7.61-7.48 (m, 2H), 7.27 (t, J = |
| | yl]piperazin-1-yl]-N-(5-[[2-(2,6- | | 7.1 Hz, 1H), 7.04-6.87 (m, 3H), 6.84 (dd, J = |
| | dioxopiperidin-3-yl)-1,3-dioxo- | | 8.4, 2.2 Hz, 1H), 5.03-4.79 (m, 1H), 4.60 (s, |
| | 2,3-dihydro-1H-isoindol-5- | | 1H), 3.27-3.23 (m, 7H), 3.13 (s, 2H), 2.78- |
| | yl]amino]pentyl)acetamide; | | 2.73 (m, 5H), 2.08-2.05 (m, 1H), 1.74-1.63 |
| | formic acid | | (m, 4H), 1.54-1.46 (m, 2H) |
| 94 | (2S,4R)-1-((S)-2-(8-(2-(4-(3- | 883.50 | ¹H NMR (300 MHz, Methanol-d4) δ 8.94 (s, |
| | amino-6-(2- | | 1H), 7.73 (s, 1H), 7.64 (dd, J = 8.1, 1.7 Hz, |
| | hydroxyphenyl)pyridazin-4-yl) | | 1H), 7.55-7.35 (m, 5H), 7.15-7.00 (m, 2H), |
| | piperazin-1- | | 4.66 (s, 1H), 4.57 (dd, J = 16.4, 8.8 Hz, 4H), |
| | yl)acetamido)octanamido)-3,3- | | 4.38 (d, J = 15.5 Hz, 1H), 4.00 (s, 2H), 3.99- |
| | dimethylbutanoyl)-4-hydroxy-N- | | 3.75 (m, 2H), 3.77-3.53 (m, 9H), 3.33 (dt, |
| | (4-(4-methylthiazol-5- | | J = 3.3, 1.7 Hz, 2H), 2.42-2.18 (m, 3H), 2.10 |
| | yl)benzyl)pyrrolidine-2- | | (ddd, J = 13.7, 9.4, 4.7 Hz, 1H), 1.74-1.47 |
| | carboxamide | | (m, 5H), 1.38 (s, 5H), 1.05 (s, 9H) |
| 95 | 2-(4-(3-amino-6-(2- | 712.50 | ¹H NMR (400 MHz, Methanol-d4) δ 7.65 (d, |
| | hydroxyphenyl)pyridazin-4- | | J = 7.7 Hz, 2H), 7.59-7.51 (m, 1H), 7.45 (t, J = |
| | yl)piperazin-1-yl)-N-(8-((2-(2,6- | | 7.6 Hz, 1H), 7.11-6.98 (m, 4H), 5.07 (dd, J = |
| | dioxopiperidin-3-yl)-1,3- | | 12.4, 5.5 Hz, 1H), 4.01-3.36 (m, 6H), 2.99- |
| | dioxoisoindolin-4- | | 2.51 (m, 4H), 2.23-1.95 (m, 2H), 1.80-1.52 |
| | yl)amino)octyl)acetamide | | (m, 5H), 1.38 (d, J = 19.2 Hz, 13H) |
| 96 | 2-(4-(3-amino-6-(2- | 685.40 | ¹H NMR (300 MHz, Methanol-d₄) δ 7.73- |
| | hydroxyphenyl)pyridazin-4- | | 7.58 (m, 3H), 7.46 (t, J = 7.7 Hz, 1H), 7.30- |
| | yl)piperazin-1-yl)-N-(2-((2-((2- | | 7.11 (m, 2H), 7.13-6.97 (m, 2H), 5.19-4.98 |
| | (2,6-dioxopiperidin-3-yl)-1,3- | | (m, 1H), 4.03-3.76 (m, 4H), 3.79-3.50 (m, |
| | dioxoisoindolin-4- | | 8H), 3.54-3.33 (m, 5H), 3.06 (d, J = 1.9 Hz, |
| | yl)amino)ethyl)(meth- | | 3H), 2.79 (m, 4H), 2.15 (s, 1H) |
| | yl)amino)ethyl)acetamide | | |
| 100 | (2S,4R)-1-[(2S)-2-(2-[2-[2-(2-[4- | 887.50 | ¹H NMR (300 MHz, Methanol-d4) δ 8.88 (s, |
| | [3-amino-6-(2- | | 1H), 7.77 (dd, J = 19.4, 9.0 Hz, 2H), 7.52 (s, |
| | hydroxyphenyl)pyridazin-4- | | 1H), 7.49-7.32 (m, 4H), 7.29 (t, J = 7.8 Hz, |
| | yl]piperazin-1- | | 1H), 6.94 (q, J = 7.3, 6.8 Hz, 2H), 4.74 (s, |
| | yl]acetamido)ethoxy]eth- | | 1H), 4.66-4.47 (m, 3H), 4.36 (d, J = 15.4 Hz, |
| | oxy]acetamido)- | | 1H), 4.05 (s, 2H), 3.86 (d, J = 11.1 Hz, 1H), |
| | 3,3-dimethylbutanoyl]-4- | | 3.83-3.71 (m, 3H), 3.71-3.51 (m, 5H), 3.55- |
| | hydroxy-N-[[4-(4-methyl-1,3- | | 3.38 (m, 1H), 3.33 (s, 6H), 3.21 (d, J = 9.6 |
| | thiazol-5- | | Hz, 5H), 3.12 (d, J = 15.7 Hz, 1H), 2.84-2.75 |
| | yl)phenyl]methyl]pyrrolidine-2- | | (m, 4H), 2.48 (s, 3H), 2.26 (dd, J = 13.0, 7.8 |
| | carboxamide | | Hz, 1H), 2.10 (ddd, J = 13.4, 9.6, 4.4 Hz, 1H), |
| | | | 1.05 (s, 9H) |
| 101 | (2R,4S)-1-[(2R)-2-[6-(2-[4-[3- | 855.45 | ¹H NMR (300 MHz, Methanol-d4) δ 8.88 (s, |
| | amino-6-(2- | | 1H), 7.88-7.73 (m, 1H), 7.55 (s, 1H), 7.51- |
| | hydroxyphenyl)pyridazin-4- | | 7.49 (M, 4H), 7.30 (td, J = 7.7, 7.1, 1.6 Hz, |
| | yl]piperazin-1- | | 1H), 7.02-6.90 (m, 2H), 4.69-4.56 (m, 4H), |
| | yl]acetamido)hexanamido]-3,3- | | 4.42-4.32 (m, 1H), 3.95-3.86 (m, 1H), 3.84- |
| | dimethylbutanoyl]-4-hydroxy-N- | | 3.76 (m, 1H), 3.33-3.22 (m, 5H), 3.17 (s, |
| | [[4-(4-methyl-1,3-thiazol-5- | | 2H), 2.85-2.79 (m, 4H), 2.48 (s, 3H), 2.40- |
| | yl)phenyl]methyl]pyrrolidine-2- | | 2.17 (m, 3H), 2.18-2.00 (m, 1H), 1.74-1.58 |
| | carboxamide | | (m, 4H), 1.46-1.39 (m, 2H), 1.04 (s, 9H) |
| 102 | (2S,4R)-1-[(2S)-2-[10-(2-[4-[3- | 911.65 | ¹H NMR (300 MHz, DMSO-d6) δ 10.58 (s, |
| | amino-6-(2- | | 1H), 9.00 (s, 1H), 8.68 (t, J = 5.6 Hz, 1H), |
| | hydroxyphenyl)pyridazin-4- | | 8.60 (t, J = 6.1 Hz, 1H), 7.87 (d, J = 9.3 Hz, |
| | yl]piperazin-1- | | 1H), 7.71-7.57 (m, 2H), 7.48 (d, J = 24.2 Hz, |
| | yl]acetamido)decanamido]-3,3- | | 1H), 7.41-7.36 (m, 6H), 7.12-6.93 (m, 2H), |
| | dimethylbutanoyl]-4-hydroxy-N- | | 5.76 (s, 1H), 4.54 (d, J = 9.4 Hz, 1H), 4.50- |
| | [[4-(4-methyl-1,3-thiazol-5- | | 4.31 (m, 3H), 4.25-4.10 (m, 1H), 4.00 (s, |
| | yl)phenyl]methyl]pyrrolidine-2- | | 2H), 3.70-3.61 (m, 6H), 3.31-3.29 (m, 2H), |
| | carboxamide | | 3.14-3.08 (m, 4H), 2.45 (s, 3H), 2.27-2.21 |
| | | | (m, 1H), 2.08-2.03 (m, 2H), 1.97-1.85 m, |
| | | | 1H), 1.46-1.44 (m, 4H), 1.25 (d, J = 4.4 Hz, |
| | | | 10H), 0.94 (s, 9H) |
| 103 | 2-[4-[3-amino-6-(2- | 656.45 | ¹H NMR (300 MHz, DMSO-d₆) δ 14.23 (s, |
| | hydroxyphenyl)pyridazin-4- | | 1H), 11.06 (s, 1H), 7.92 (dd, J = 8.4, 1.6 Hz, |
| | yl]piperazin-1-yl]-N-(4-[[2-(2,6- | | 1H), 7.84 (t, J = 6.0 Hz, 1H), 7.56 (d, J = 8.4 |
| | dioxopiperidin-3-yl)-1,3-dioxo- | | Hz, 1H), 7.50 (s, 1H), 7.31-7.19 (m, 1H), |
| | 2,3-dihydro-1H-isoindol-5- | | 7.14 (t, J = 5.3 Hz, 1H), 6.99-6.81 (m, 4H), |
| | yl]amino]butyl)acetamide | | 6.26 (s, 2H), 5.02-4.95 (m, 1H), 3.16 (s, 8H), |
| | | | 3.01 (s, 2H), 2.96-2.78 (m, 1H), 2.67 (s, 4H), |
| | | | 2.60 (s, 1H), 2.01-1.98 (m, 1H), 1.56 (s, 4H) |

### Example 3. Preparation of 4-(2-[[3-amino-6-(2-hydroxyphenyl)pyridazin-4-yl]oxy]ethyl)-N-(5-[[2-(2,6-dioxopiperidin-3-yl)-1,3-dioxoisoindol-4-yl]oxy]pentyl)benzamide (compound 21)

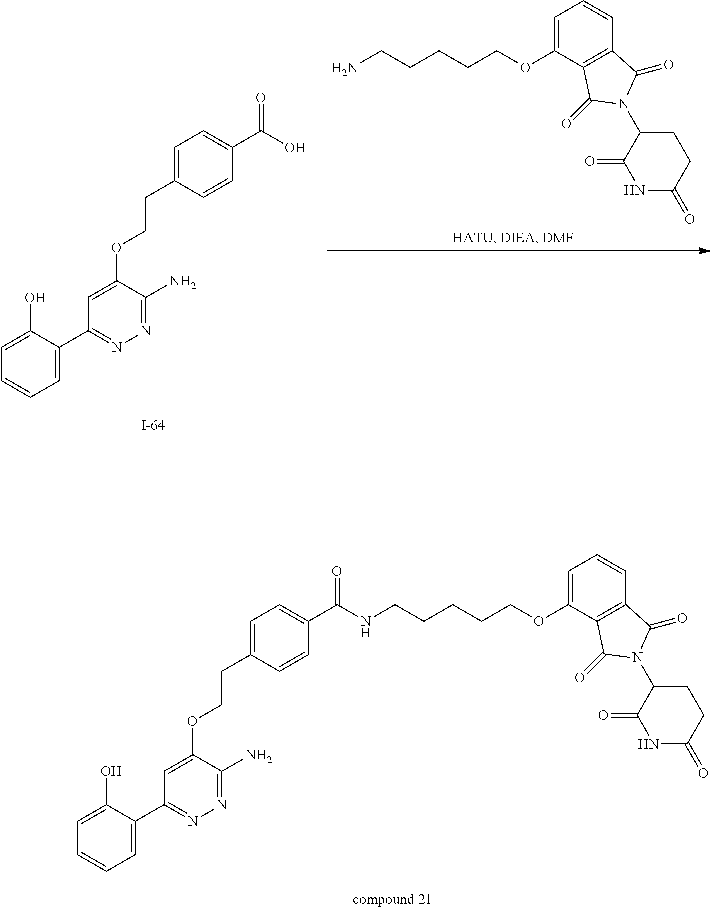

To a stirred mixture of 4-(2-((3-amino-6-(2-hydroxyphenyl)pyridazin-4-yl)oxy)ethyl)benzoic acid (I-64, 20.00 mg, 0.057 mmol, 1.00 equiv) and 4-[(5-aminopentyl)oxy]-2-(2,6-dioxopiperidin-3-yl)isoindole-1,3-dione (24.55 mg, 0.068 mmol, 1.20 equiv) in DMF (1.5 mL) was added HATU (28.14 mg, 0.074 mmol, 1.30 equiv) and DIEA (22.07 mg, 0.171 mmol, 3.00 equiv) at room temperature, and the reaction was stirred for 2 h under an atmosphere of dry nitrogen. The reaction mixture was directly purified by reversed-phase preparative HPLC to afford 4-(2-[[3-amino-6-(2-hydroxyphenyl)pyridazin-4-yl]oxy]ethyl)-N-(5-[[2-(2,6-dioxopiperidin-3-yl)-1,3-dioxoisoindol-4-yl]oxy]pentyl)benzamide (compound 21, 15.3 mg, 37.7 5%) as a white solid. ¹H NMR (300 MHz, DMSO-d₆) δ 7.92-7.81 (m, 1H), 7.78 (dd, J=8.0, 2.9 Hz, 3H), 7.57 (s, 1H), 7.53-7.36 (m, 4H), 7.33-7.20 (m, 1H), 6.90 (dt, J=7.1, 3.2 Hz, 2H), 6.62 (s, 1H), 5.06 (dd, J=12.8, 5.4 Hz, 1H), 4.49 (t, J=66.6 Hz, 2H), 4.20 (t, J=6.3 Hz, 2H), 3.23 (dt, J=21.8, 6.6 Hz, 4H), 2.94-2.71 (m, 1H), 2.55-2.40 (m, 1H), 2.10-1.94 (m, 1H), 1.83-1.74 (m, 2H), 1.68-1.40 (m, 4H), 1.27-1.06 (m, 1H). LCMS (ESI) m/z: [M+H]+=693.30.

The following compounds in Table C1 were prepared using procedures similar to those used for the preparation of compound 21.

TABLE C1

| | | LCMS | |
|---|---|---|---|
| | | (ESI) | |
| No. | Name | m/z | ¹H NMR |
| 18 | 2-[[4-(2-[[3-amino-6-(2- | 726.4 | ¹H NMR (400 MHz, DMSO-d6) δ 14.38 (s, 1H), |
| | hydroxyphenyl)pyridazin-4- | | 11.04 (s, 1H), 8.76 (t, J = 5.9 Hz, 1H), 8.47 (t, J = |
| | yl]oxy]ethyl)phenyl]formamido]- | | 6.0 Hz, 1H), 8.22 (s, 1H), 7.95 (dd, J = 8.3, 1.7 |
| | N-([4-[(3-chloro-1H-indol-7- | | Hz, 1H), 7.86 (d, J = 8.1 Hz, 2H), 7.74-7.66 (m, |
| | yl)sulfamoyl]phe- | | 2H), 7.62 (s, 1H), 7.52 (d, J = 8.0 Hz, 2H), 7.45 (s, |
| | nyl]methyl)acetamide | | 1H), 7.37 (d, J = 8.1 Hz, 2H), 7.24 (td, J = 7.6, 1.6 |
| | | | Hz, 1H), 7.17 (d, J = 7.9 Hz, 1H), 6.97-6.77 (m, |
| | | | 4H), 6.52 (s, 2H), 5.76 (s, 1H), 4.49 (t, J = 6.6 Hz, |
| | | | 2H), 4.31 (d, J = 5.8 Hz, 2H), 3.90 (d, J = 5.7 Hz, |
| | | | 2H), 3.21 (t, J = 6.6 Hz, 2H) |
| 24 | (2S,4R)-1-[(2S)-2-[4-[2-(2- | 937.25 | ¹H NMR (300 MHz, DMSO-d6) δ 14.34 (s, 1H), |
| | [[4-(2-[[3-amino-6-(2- | | 8.99 (s, 1H), 8.54 (dt, J = 27.7, 5.8 Hz, 2H), 7.98- |
| | hydroxyphenyl)pyridazin-4- | | 7.79 (m, 4H), 7.62 (s, 1H), 7.50 (d, J = 8.0 Hz, |
| | yl]oxy]ethyl)phe- | | 2H), 7.45-7.36 (m, 4H), 7.24 (td, J = 7.6, 1.5 Hz, |
| | nyl]formamido]ethoxy)eth- | | 1H), 6.92-6.84 (m, 2H), 6.55 (d, J = 6.8 Hz, 2H), |
| | oxy]butanamido]-3,3- | | 5.15 (d, J = 3.5 Hz, 1H), 4.57-4.33 (m, 6H), 4.22 |
| | dimethylbutanoyl]-4- | | (dd, J = 15.9, 5.3 Hz, 1H), 3.66 (s, 2H), 3.53 (p, |
| | hydroxy-N-[[4-(4-methyl-1,3- | | J = 3.5, 2.9 Hz, 4H), 3.49-3.43 (m, 3H), 3.42 (s, |
| | thiazol-5- | | 2H), 3.40 (s, 1H), 3.20 (t, J = 6.7 Hz, 2H), 2.44 (s, |
| | yl)phenyl]methyl]pyrrolidine- | | 3H), 2.22 (dq, J = 24.4, 7.2 Hz, 2H), 2.04 (t, J = |
| | 2-carboxamide | | 10.3 Hz, 1H), 1.95-1.84 (m, 1H), 1.69 (h, J = 6.7 |
| | | | Hz, 2H), 0.93 (s, 9H) |
| 41 | N-(8-[[4-(2-[[3-amino-6-(2- | 791.86 | ¹H NMR (300 MHz, DMSO-d6) δ 11.14 (s, 1H), |
| | hydroxyphenyl)pyridazin-4- | | 8.40 (t, J = 5.5 Hz, 1H), 8.02-7.87 (m, 1H), 7.80 |
| | yl]oxy]ethyl)phe- | | (d, J = 7.7 Hz, 3H), 7.69 (d, J = 10.3 Hz, 2H), 7.58- |
| | nyl]formamido]octyl)-2-[[2-(2,6- | | 7.45 (m, 3H), 7.44-7.18 (m, 3H), 6.97 (q, J = |
| | dioxopiperidin-3-yl)-1,3- | | 7.7, 7.2 Hz, 2H), 5.13 (dd, J = 13.1, 5.4 Hz, 1H), |
| | dioxoisoindol-4- | | 4.77 (s, 2H), 4.57 (t, J = 6.7 Hz, 2H), 3.17 (dt, J = |
| | yl]oxy]acetamide | | 27.0, 6.6 Hz, 6H), 3.02-2.76 (m, 2H), 2.68 (d, J = |
| | | | 33.9 Hz, 3H), 2.08 (m, 1H), 1.46 (m, 4H), 1.27 (m, |
| | | | 8H) |
| 42 | 4-(2-[[3-amino-6-(2- | 678.20 | ¹H NMR (300 MHz, Methanol-d4) δ 7.79-7.70 |
| | hydroxyphenyl)pyridazin-4- | | (m, 2H), 7.67-7.56 (m, 2H), 7.55-7.38 (m, 4H), |
| | yl]oxy]ethyl)-N-(4-[[2-(2,6- | | 7.11-6.97 (m, 3H), 6.85 (dd, J = 8.4, 2.5 Hz, 1H), |
| | dioxopiperidin-3-yl)-1,3- | | 5.03 (dd, J = 12.4, 5.5 Hz, 1H), 4.69 (t, J = 6.6 Hz, |
| | dioxoisoindol-5- | | 2H), 3.44 (d, J = 5.9 Hz, 2H), 3.33 (p, J = 1.7 Hz, |
| | yl]amino]butyl)benzamide | | 4H), 2.96-2.56 (m, 3H), 2.18-1.95 (m, 1H), |
| | | | 1.76 (p, J = 3.3 Hz, 4H) |
| 43 | 4-(2-[[3-amino-6-(2- | 738.20 | ¹H NMR (300 MHz, DMSO-d₆) δ 11.10 (s, 1H), |
| | hydroxyphenyl)pyridazin-4- | | 8.45 (t, J = 5.6 Hz, 1H), 7.81 (d, J = 8.0 Hz, 2H), |
| | yl]oxy]ethyl)-N-[2-[2-(2-[[2- | | 7.67 (d, J = 10.6 Hz, 2H), 7.63-7.55 (m, 1H), |
| | (2,6-dioxopiperidin-3-yl)-1,3- | | 7.48 (d, J = 8.1 Hz, 2H), 7.34 (d, J = 7.9 Hz, 3H), |
| | dioxoisoindol-4- | | 7.11 (d, J = 8.4 Hz, 1H), 7.09-6.85 (m, 3H), 6.60 |
| | yl]amino]ethoxy)eth- | | (s, 1H), 5.05 (dd, J = 12.6, 5.3 Hz, 1H), 4.56 (t, J = |
| | oxy]ethyl]benzamide | | 6.4 Hz, 2H), 3.70-3.49 (m, 11H), 3.21 (t, J = 6.7 |
| | | | Hz, 4H), 2.98-2.78 (m, 1H), 2.54 (s, 2H), 2.04 (t, |
| | | | J = 12.6 Hz, 1H) |
| 44 | 4-(2-((3-amino-6-(2- | 734.25 | ¹H NMR (300 MHz, DMSO-d6) δ 14.36 (brs, 1H), |
| | hydroxyphenyl)pyridazin-4- | | 11.10 (s, 1H), 8.38 (t, J = 5.6 Hz, 1H), 8.01-7.89 |
| | yl)oxy)ethyl)-N-(8-((2-(2,6- | | (m, 1H), 7.80 (d, J = 8.2 Hz, 2H), 7.66-7.53 (m, |
| | dioxopiperidin-3-yl)-1,3- | | 2H), 7.54-7.45 (m, 2H), 7.30-7.21 (m, 1H), 7.09 |
| | dioxoisoindolin-4- | | (d, J = 8.6 Hz, 1H), 7.02 (d, J = 7.0 Hz, 1H), 6.96- |
| | yl)amino)octyl)benzamide | | 6.80 (m, 2H), 6.73-6.42 (m, 3H), 5.05 (dd, J = |
| | | | 12.8, 5.4 Hz, 1H), 4.49 (t, J = 6.7 Hz, 2H), 3.32- |
| | | | 3.10 (m, 6H), 2.95-2.80 (m, 1H), 2.62-2.58 (m, |
| | | | 1H), 2.13-1.91 (m, 1H), 1.65-1.42 (d, J = 10.2 |
| | | | Hz, 4H), 1.31 (s, 9H) |
| 45 | 4-(2-[[3-amino-6-(2- | 747.40 | ¹H NMR (400 MHz, DMSO-d6) δ 14.27 (brs, |
| | hydroxyphenyl)pyridazin-4- | | 1H), 11.00 (s, 1H), 8.38 (d, J = 5.9 Hz, 1H), 8.30 |
| | yl]oxy]ethyl)-N-(8-[[3-(2,6- | | (d, J = 5.3 Hz, 1H), 7.93 (d, J = 7.7 Hz, 1H), 7.80 |
| | dioxopiperidin-3-yl)-2- | | (d, J = 8.0 Hz, 2H), 7.62 (s, 1H), 7.48 (t, J = 8.4 |
| | methyl-4-oxoquinazolin-5- | | Hz, 3H), 7.25 (t, J = 7.7 Hz, 1H), 6.88 (t, J = 7.8 |
| | yl]amino]octyl)benzamide | | Hz, 2H), 6.64 (s, 1H), 6.56 (s, 2H), 6.49 (d, J = 8.5 |
| | | | Hz, 1H), 5.18 (dd, J = 11.4, 5.7 Hz, 1H), 4.49 (t, |
| | | | J = 6.8 Hz, 2H), 3.30-3.04 (m, 6H), 2.81 (d, J = |
| | | | 15.7 Hz, 1H), 2.69-2.58 (m, 5H), 2.14 (d, J = |
| | | | 10.2 Hz, 1H), 1.77-1.49 (m, 4H), 1.49-1.06 (m, |
| | | | 8H) |
| 46 | 4-(2-[[3-amino-6-(2- | 706.20 | ¹H NMR (300 MHz, DMSO-d6) δ 11.07 (s, 1H), |
| | hydroxyphenyl)pyridazin-4- | | 8.41 (t, J = 5.6 Hz, 1H), 7.87 (d, J = 7.9 Hz, 1H), |
| | yl]oxy]ethyl)-N-(6-[[2-(2,6- | | 7.85-7.74 (m, 2H), 7.63 (s, 1H), 7.61-7.39 (m, |
| | dioxopiperidin-3-yl)-1,3- | | 3H), 7.32-7.22 (m, 1H), 7.11 (t, J = 5.4 Hz, 1H), |
| | dioxoisoindol-5- | | 6.99-6.81 (m, 4H), 6.75 (s, 2H), 5.03 (dd, J = |
| | yl]amino]hexyl)benzamide | | 12.8, 5.4 Hz, 1H), 4.51 (t, J = 6.7 Hz, 2H), 3.52- |
| | | | 3.09 (m, 7H), 2.95-2.82 (m, 1H), 2.65-2.55 (m, |
| | | | 1H), 2.05-1.94 (m, 1H), 1.69-1.47 (m, 4H), |
| | | | 1.46-1.29 (m, 4H), 1.26-1.18 (m, J = 6.4, 3.8, 2.2 |
| | | | Hz, 1H) |
| 47 | (2S,4R)-1-[(2S)-2-(10-[[4-(2- | 933.5 | ¹H NMR (300 MHz, DMSO-d6) δ 14.35 (s, 1H), |
| | [[3-amino-6-(2- | | 8.98 (s, 1H), 8.53 (t, J = 6.0 Hz, 1H), 8.36 (t, J = |
| | hydroxyphenyl)pyridazin-4- | | 5.5 Hz, 1H), 7.94 (dd, J = 8.4, 1.6 Hz, 1H), 7.80 |
| | yl]oxy]ethyl)phe- | | (d, J = 8.4 Hz, 3H), 7.61 (s, 1H), 7.54-7.45 (m, |
| | nyl]formamido]decanamido)-3,3- | | 2H), 7.46-7.33 (m, 4H), 7.24 (td, J = 7.5, 1.6 Hz, |
| | dimethylbutanoyl]-4-hydroxy- | | 1H), 6.91-6.85 (m, 2H), 6.49 (s, 2H), 5.10 (d, J = |
| | N-[[4-(4-methyl-1,3-thiazol-5- | | 3.5 Hz, 1H), 4.66-4.31 (m, 6H), 4.25-4.18 (m, |
| | yl)phenyl]methyl]pyrrolidine- | | 1H), 3.66 (d, J = 4.5 Hz, 2H), 3.21 (q, J = 6.8 Hz, |
| | 2-carboxamide | | 4H), 2.45 (s, 3H), 2.36-1.82 (m, 4H), 1.60-1.33 |
| | | | (m, 4H), 1.32-1.20 (m, 10H), 0.94 (s, 9H) |
| 48 | 4-(2-[[3-amino-6-(2- | 751.20 | ¹H NMR (300 MHz, DMSO-d6) δ 11.00 (s, 1H), |
| | hydroxyphenyl)pyridazin-4- | | 8.42 (q, J = 5.9 Hz, 2H), 7.94 (d, J = 7.5 Hz, 1H), |
| | yl]oxy]ethyl)-N-[2-[2-(2-[[3- | | 7.81 (d, J = 8.1 Hz, 2H), 7.61 (s, 1H), 7.56-7.35 |
| | (2,6-dioxopiperidin-3-yl)-2- | | (m, 3H), 7.24 (t, J = 7.1 Hz, 1H), 6.87 (dd, J = 8.0, |
| | methyl-4-oxoquinazolin-5- | | 6.7 Hz, 2H), 6.70-6.60 (m, 1H), 6.59-6.46 (m, |
| | yl]amino]ethoxy)etho- | | 3H), 5.17 (dd, J = 11.0, 5.7 Hz, 1H), 4.48 (t, J = |
| | xy]ethyl]benzamide | | 6.7 Hz, 2H), 3.63 (t, J = 5.5 Hz, 2H), 3.53 (d, J = |
| | | | 5.8 Hz, 3H), 3.41 (t, J = 5.8 Hz, 2H), 3.34 (s, 3H), |
| | | | 3.38-3.19 (m, 5H), 2.84-2.57 (m, 1H), 2.52 (m, |
| | | | 5H), 2.14-2.03 (m, 1H) |
| 49 | 4-(2-(1′-(4-(2-((3-amino-6-(2- | 816.25 | ¹H NMR (400 MHz, DMSO-d6) δ 14.38 (brs, 1H), |
| | hydroxyphenyl)pyridazin-4- | | 11.11 (s, 1H), 7.94 (d, J = 8.2 Hz, 1H), 7.77 (dd, |
| | yl)oxy)ethyl)benzoyl)-[4,4′- | | J = 8.6, 7.3 Hz, 1H), 7.61 (s, 1H), 7.46 (dd, J = 9.5, |
| | bipiperidin]-1-yl)-2- | | 7.6 Hz, 3H), 7.32 (d, J = 7.7 Hz, 2H), 7.28-7.20 |
| | oxoethoxy)-2-(2,6- | | (m, 1H), 6.94-6.80 (m, 2H), 6.53 (d, J = 4.9 Hz, |
| | dioxopiperidin-3- | | 2H), 5.18 (s, 1H), 5.10 (dd, J = 12.7, 5.4 Hz, 2H), |
| | yl)isoindoline-1,3-dione | | 4.51 (t, J = 6.8 Hz, 3H), 4.34 (d, J = 12.8 Hz, 1H), |
| | | | 3.83 (d, J = 13.0 Hz, 1H), 3.19 (t, J = 6.8 Hz, 2H), |
| | | | 2.93 (dd, J = 39.5, 12.8 Hz, 3H), 2.62-2.52 (m, |
| | | | 3H), 2.14-1.95 (m, 1H), 1.70-1.60 (m, 4H), 1.30- |
| | | | 1.28 (m, 4H), 1.23-0.94 (m, 4H) |
| 50 | 4-(2-((3-amino-6-(2- | 692.15 | ¹H NMR (400 MHz, DMSO-d6) δ 14.32 (brs, 1H), |
| | hydroxyphenyl)pyridazin-4- | | 11.10 (s, 1H), 8.41 (t, J = 5.7 Hz, 1H), 8.01-7.87 |
| | yl)oxy)ethyl)-N-(5-((2-(2,6- | | (m, 1H), 7.86-7.74 (m, 2H), 7.61 (s, 1H), 7.57 |
| | dioxopiperidin-3-yl)-1,3- | | (dd, J = 8.6, 7.1 Hz, 1H), 7.49 (d, J = 8.2 Hz, 2H), |
| | dioxoisoindolin-4- | | 7.24 (td, J = 7.6, 1.6 Hz, 1H), 7.10 (d, J = 8.6 Hz, |
| | yl)amino)pentyl)benzamide | | 1H), 7.02 (d, J = 7.0 Hz, 1H), 6.88 (dd, J = 7.9, 6.5 |
| | | | Hz, 2H), 6.63-6.37 (m, 3H), 5.05 (dd, J = 12.9, |
| | | | 5.4 Hz, 1H), 4.49 (t, J = 6.7 Hz, 2H), 3.25-3.18 (m, |
| | | | 6H), 2.89-2.82 (m, 2H), 2.74-2.53 (m, 3H), 2.17- |
| | | | 1.94 (m, 1H), 1.80-1.60 (m, 4H), 1.47-1.30 (m, |
| | | | 2H) |
| 51 | 4-(2-[[3-amino-6-(2- | 692.20 | ¹H NMR (300 MHz, DMSO-d6) δ 11.06 (brs, 1H), |
| | hydroxyphenyl)pyridazin-4- | | 8.41 (t, J = 5.6 Hz, 1H), 7.88-7.75 (m, 2H), 7.78- |
| | yl]oxy]ethyl)-N-(5-[[2-(2,6- | | 7.69 (m, 1H), 7.56 (d, J = 8.4 Hz, 1H), 7.52 (d, |
| | dioxopiperidin-3-yl)-1,3- | | J = 8.0 Hz, 1H), 7.49 (d, J = 8.2 Hz, 2H), 7.35 (t, J = |
| | dioxoisoindol-5- | | 7.6 Hz, 1H), 7.10 (d, J = 6.4 Hz, 1H), 6.96 (td, J = |
| | yl]amino]pentyl)benzamide. | | 7.9, 5.6 Hz, 3H), 6.85 (dd, J = 8.4, 2.1 Hz, 1H), |
| | | | 5.03 (dd, J = 12.8, 5.4 Hz, 1H), 4.56 (t, J = 6.6 Hz, |
| | | | 2H), 3.27 (m, 3H), 3.39-3.01 (m, 6H), 3.04- |
| | | | 2.80 (m, 1H), 2.51-2.49 (m, 1H), 2.10-1.90 (m, |
| | | | 1H), 1.60 (dt, J = 14.0, 7.0 Hz, 4H), 1.45-1.19 |
| | | | (m, 2H) |
| 52 | 4-(2-[[3-amino-6-(2- | 719.20 | ¹H NMR (300 MHz, DMSO-d6) δ 14.37 (s, 1H), |
| | hydroxyphenyl)pyridazin-4- | | 10.99 (s, 1H), 8.46-8.35 (m, 1H), 8.32 (t, J = 5.2 |
| | yl]oxy]ethyl)-N-(6-[[3-(2,6- | | Hz, 1H), 7.99-7.89 (m, 1H), 7.84-7.77 (m, 2H), |
| | dioxopiperidin-3-yl)-2- | | 7.62 (s, 1H), 7.48 (t, J = 7.9 Hz, 3H), 7.28-7.19 |
| | methyl-4-oxoquinazolin-5- | | (m, 1H), 6.93-6.81 (m, 2H), 6.63 (d, J = 7.8 Hz, |
| | yl]amino]hexyl)benzamide | | 1H), 6.50 (d, J = 9.4 Hz, 3H), 5.22-5.13 (m, 1H), |
| | | | 4.49 (t, J = 6.7 Hz, 2H), 3.27-3.12 (m, 6H), 2.94- |
| | | | 2.68 (m, 2H), 2.58 (d, J = 15.4 Hz, 4H), 2.23- |
| | | | 2.02 (m, 1H), 1.73-1.48 (m, 4H), 1.38 (s, 4H) |
| 53 | (2R,4S)-1-((R)-2-(6-(4-(2-((3- | 877.25 | ¹H NMR (400 MHz, DMSO-d6) δ 8.99 (s, 1H), |
| | amino-6-(2- | | 8.56 (t, J = 6.2 Hz, 1H), 8.38 (t, J = 5.7 Hz, 1H), |
| | hydroxyphenyl)pyridazin-4- | | 7.91-7.73 (m, 4H), 7.63 (s, 1H), 7.55-7.46 (m, |
| | yl)oxy)ethyl)benzamido)hexanamido)- | | 2H), 7.40 (q, J = 8.3 Hz, 4H), 7.29 (t, J = 7.6 Hz, |
| | 3,3-dimethylbutanoyl)-4- | | 1H), 7.00-6.83 (m, 2H), 6.80-6.63 (m, 1H), |
| | hydroxy-N-(4-(4- | | 5.13 (s, 1H), 4.60-4.48 (m, 3H), 4.48-4.39 (m, |
| | methylthiazol-5- | | 2H), 4.35 (s, 1H), 4.22 (dd, J = 15.9, 5.4 Hz, 1H), |
| | yl)benzyl)pyrrolidine-2- | | 3.74-3.57 (m, 2H), 3.36-3.18 (m, 3H), 2.45 (s, |
| | carboxamide | | 3H), 2.27 (dt, J = 14.6, 7.5 Hz, 1H), 2.13 (dt, J = |
| | | | 14.2, 7.1 Hz, 1H), 2.03 (t, J = 10.4 Hz, 1H), 1.90 |
| | | | (m, 1H), 1.60-1.43 (m, 4H), 1.28 (dt, J = 15.4, |
| | | | 7.7 Hz, 2H), 0.93 (s, 9H) |
| 54 | 4-(2-[[3-amino-6-(2- | 724.20 | ¹H NMR (300 MHz, DMSO-d6) δ 11.00 (s, 1H), |
| | hydroxyphenyl)pyridazin-4- | | 8.46 (t, J = 5.6 Hz, 1H), 7.91 (s, 1H), 7.85-7.73 |
| | yl]oxy]ethyl)-N-[2-[2-(2-[[2- | | (m, 2H), 7.62 (s, 1H), 7.49 (d, J = 8.2 Hz, 2H), |
| | (2,6-dioxopiperidin-3-yl)-1- | | 7.34-7.18 (m, 2H), 6.98-6.84 (m, 3H), 6.79 (d, |
| | oxo-3H-isoindol-4- | | J = 8.0 Hz, 1H), 6.64 (s, 1H), 5.58 (s, 1H), 5.11 |
| | yl]amino]ethoxy)eth- | | (dd, J = 13.2, 5.1 Hz, 1H), 4.50 (t, J = 6.7 Hz, 2H), |
| | oxy]ethyl]benzamide | | 4.17 (q, J = 17.2 Hz, 2H), 3.75-3.46 (m, 8H), |
| | | | 3.42 (t, J = 5.7 Hz, 4H), 3.20 (t, J = 6.7 Hz, 2H), |
| | | | 2.99-2.82 (m, 1H), 2.75-2.71 (m, 1H), 2.60 (d, |
| | | | J = 17.3 Hz, 1H), 2.40-2.16 (m, 1H), 2.11-1.92 |
| | | | (m, 1H) |
| 55 | 4-(2-[[3-amino-6-(2- | 742.10 | ¹H NMR (300 MHz, DMSO-d6) ¹H NMR (300 |
| | hydroxyphenyl)pyridazin-4- | | MHz, DMSO-d6) δ 11.10 (s, 1H), 8.68 (t, J = 5.7 |
| | yl]oxy]ethyl)-N-[2-(2-[[2-(2,6- | | Hz, 1H), 7.83-7.75 (m, 2H), 7.72 (d, J = 6.6 Hz, |
| | dioxopiperidin-3-yl)-1,3- | | 1H), 7.66 (s, 2H), 7.60 (dd, J = 8.5, 7.1 Hz, 1H), |
| | dioxoisoindol-4- | | 7.50 (d, J = 8.3 Hz, 1H), 7.35 (t, J = 7.8 Hz, 1H), |
| | yl]amino]ethane- | | 7.16 (d, J = 8.6 Hz, 1H), 7.08 (d, J = 7.0 Hz, 1H), |
| | sulfonyl)ethyl]benzamide | | 6.96 (dd, J = 13.2, 7.4 Hz, 2H), 6.86 (t, J = 6.2 Hz, |
| | | | 1H), 5.06 (dd, J = 12.8, 5.4 Hz, 1H), 4.56 (t, J = |
| | | | 6.7 Hz, 2H), 3.78 (t, J = 6.4 Hz, 2H), 3.69 (d, J = |
| | | | 6.2 Hz, 2H), 3.50-3.46 (m, 5H), 3.22 (t, J = 6.6 Hz, |
| | | | 2H), 3.04-2.80 (m, 1H), 2.61 (s, 1H), 2.17-1.8 |
| | | | (m, 1H) |
| 56 | (4-(2-[[3-amino-6-(2- | 720.20 | ¹H NMR (300 MHz, DMSO-d6) δ 11.01 (s, 1H), |
| | hydroxyphenyl)pyridazin-4- | | 8.58-8.21 (m, 1H), 8.05-7.88 (m, 1H), 7.80 (d, |
| | yl]oxy]ethyl)-N-(8-[[2-(2,6- | | J = 8.0 Hz, 2H), 7.61 (s, 1H), 7.49 (d, J = 8.0 Hz, |
| | dioxopiperidin-3-yl)-1-oxo- | | 2H), 7.36-7.12 (m, 2H), 7.01-6.77 (m, 3H), |
| | 3H-isoindol-4- | | 6.74 (d, J = 8.0 Hz, 1H), 6.51 (s, 2H), 5.55 (t, J = |
| | yl]amino]octyl)benzamide | | 5.5 Hz, 1H), 5.11 (dd, J = 13.2, 5.1 Hz, 1H), 4.49 |
| | | | (t, J = 6.7 Hz, 2H), 4.18 (q, J = 17.2 Hz, 2H), 3.27- |
| | | | 3.03 (m, 5H), 2.93 (ddd, J = 18.1, 13.5, 5.3 Hz, |
| | | | 1H), 2.78-2.56 (m, 2H), 2.40-2.19 (m, 1H), |
| | | | 2.13-1.94 (m, 1H), 1.55 (dt, J = 19.0, 6.7 Hz, |
| | | | 4H), 1.45-1.20 (m, 8H) |
| 57 | (2S,4R)-1-[(2S)-2-(8-[[4-(2- | 905.35 | ¹H NMR (400 MHz, DMSO-d6) δ 8.98 (s, 1H), |
| | [[3-amino-6-(2- | | 8.56 (t, J = 6.1 Hz, 1H), 8.38 (t, J = 5.7 Hz, 1H), |
| | hydroxyphenyl)pyridazin-4- | | 7.98-7.91 (m, 1H), 7.90-7.78 (m, 3H), 7.61 (s, |
| | yl]oxy]ethyl)phe- | | 1H), 7.49 (d, J = 8.1 Hz, 2H), 7.45-7.35 (m, 4H), |
| | nyl]formamido]octanamido)-3,3- | | 7.27-7.21 (m, 1H), 6.92-6.84 (m, 2H), 6.51 (s, |
| | dimethylbutanoyl]-4-hydroxy- | | 2H), 5.12 (d, J = 3.5 Hz, 1H), 4.65-4.08 (m, 7H), |
| | N-[[4-(4-methyl-1,3-thiazol-5- | | 3.81-3.58 (m, 2H), 3.28-3.16 (m, 4H), 2.45 (s, |
| | yl)phenyl]methyl]pyrrolidine- | | 3H), 2.20-2.30 (m, 1H), 2.15 (s, 2H), 1.95-1.87 |
| | 2-carboxamide | | (m, 1H), 1.58-1.41 (m, 4H), 1.33-1.18 (m, 6H), |
| | | | 0.93 (s, 9H) |
| 58 | N-[2-[(2-[[4-(2-[[3-amino-6- | 765.50 | ¹H NMR (300 MHz, Methanol-d4) δ 8.38 (s, |
| | (2-hydroxyphenyl)pyridazin- | | 0.72H), 7.79 (d, J = 8.0 Hz, 1H), 7.73-7.63 (m, |
| | 4-yl]oxy]ethyl)phe- | | 3H), 7.50-7.39 (m, 2H), 7.29 (dd, J = 8.6, 3.2 Hz, |
| | nyl]formamido]ethyl)(meth- | | 4H), 7.00-6.87 (m, 2H), 5.11 (dd, J = 12.6, 5.4 |
| | yl)amino]ethyl]-2-[[2-(2,6- | | Hz, 1H), 4.72-4.50 (m, 2H), 4.47 (t, J = 6.4 Hz, |
| | dioxopiperidin-3-yl)-1,3- | | 2H), 3.56 (d, J = 5.5 Hz, 4H), 3.17 (t, J = 6.4 Hz, |
| | dioxoisoindol-4- | | 2H), 2.91 (d, J = 5.0 Hz, 4H), 2.84-2.61 (m, 3H), |
| | yl]oxy]acetamide | | 2.60 (s, 3H), 2.10 (d, J = 12.7 Hz, 1H) |
| 59 | (2R,4S)-1-[(2R)-2-[2-[2-(2- | 909.55 | ¹H NMR (300 MHz, Methanol-d4) δ 8.86 (s, 1H), |
| | [[4-(2-[[3-amino-6-(2- | | 8.54 (d, J = 6.2 Hz, 1H), 7.84-7.71 (m, 3H), 7.49 |
| | hydroxyphenyl)pyridazin-4- | | (q, J = 3.5 Hz, 3H), 7.36 (s, 3H), 7.28 (td, J = 7.5, |
| | yl]oxy]ethyl)phe- | | 1.5 Hz, 1H), 7.02-6.83 (m, 2H), 4.80-4.70 (m, |
| | nyl]formamido]ethoxy)eth- | | 1H), 4.68-4.35 (m, 5H), 4.34-4.18 (m, 1H), |
| | oxy]acetamido]-3,3- | | 4.13-3.79 (m, 4H), 3.79-3.50 (m, 8H), 3.28 (t, |
| | dimethylbutanoyl]-4- | | J = 7.2 Hz, 2H), 2.46 (d, J = 8.6 Hz, 3H), 2.25-2.15 |
| | hydroxy-N-[[4-(4-methyl-1,3- | | (m, 1H), 2.08-2.01 (m, 1H), 1.02 (s, 9H) |
| | thiazol-5- | | |
| | yl)phenyl]methyl]pyrrolidine- | | |
| | 2-carboxamide | | |
| 60 | (2R,4S)-1-[(2R)-2-(2-[2-[2- | 953.45 | ¹H NMR (300 MHz, Methanol-d4) δ 8.87 (s, 1H), |
| | (2-[[4-(2-[[3-amino-6-(2- | | 7.79 (dd, J = 8.2, 2.5 Hz, 3H), 7.55-7.34 (m, 7H), |
| | hydroxyphenyl)pyridazin-4- | | 7.32-7.19 (m, 1H), 6.92 (d, J = 7.7 Hz, 2H), 4.75- |
| | yl]oxy]ethyl)phe- | | 4.65 (m, 1H), 4.65-4.46 (m, 5H), 4.34 (d, J = |
| | nyl]formamido]ethoxy)eth- | | 15.6 Hz, 1H), 4.10-3.76 (m, 4H), 3.66 (ddt, J = |
| | oxy]ethoxy]acetamido)-3,3- | | 10.7, 8.5, 3.7 Hz, 10H), 3.61-3.41 (m, 2H), 3.27 |
| | dimethylbutanoyl]-4-hydroxy- | | (d, J = 6.4 Hz, 2H), 2.48 (s, 3H), 2.24 (dd, J = |
| | N-[[4-(4-methyl-1,3-thiazol-5- | | 13.3, 7.5 Hz, 1H), 2.08 (ddd, J = 13.2, 9.2, 4.4 Hz, |
| | yl)phenyl]methyl]pyrrolidine- | | 1H), 1.04 (s, 9H) |
| | 2-carboxamide | | |
| 61 | 4-(2-[[3-amino-6-(2- | 707.40 | ¹H NMR (300 MHz, Methanol-d4) δ 7.83 (d, J = |
| | hydroxyphenyl)pyridazin-4- | | 8.2 Hz, 1H), 7.67 (d, J = 7.9 Hz, 2H), 7.57-7.44 |
| | yl]oxy]ethyl)-N-[2-[(2-[[2-(2,6- | | (m, 2H), 7.38 (d, J = 8.0 Hz, 2H), 7.26 (t, J = 7.6 |
| | dioxopiperidin-3-yl)-1,3- | | Hz, 1H), 7.05 (d, J = 8.5 Hz, 1H), 6.93 (q, J = 7.3, |
| | dioxoisoindol-4- | | 6.6 Hz, 3H), 5.01 (dd, J = 12.4, 5.4 Hz, 1H), 4.52 |
| | yl]amino]ethyl)(meth- | | (t, J = 6.6 Hz, 2H), 3.65-3.52 (m, 4H), 3.25 (t, |
| | yl)amino]ethyl]benzamide | | J = 6.7 Hz, 2H), 3.04 (d, J = 14.8 Hz, 4H), 2.84- |
| | | | 2.61 (m, 5H), 2.06 (m, 1H) |
| 64 | 4-(2-[[3-amino-6-(2- | 734.55 | ¹H NMR (300 MHz, Methanol-d4) δ 7.83-7.71 |
| | hydroxyphenyl)pyridazin-4- | | (m, 2H), 7.68-7.36 (m, 6H), 7.09-6.99 (m, 2H), |
| | yl]oxy]ethyl)-N-(8-[[2-(2,6- | | 6.96 (d, J = 2.1 Hz, 1H), 6.85-6.79 (m, 1H), 5.08- |
| | dioxopiperidin-3-yl)-1,3- | | 4.98 (m, 1H), 4.68 (t, J = 6.6 Hz, 2H), 3.38 (t, |
| | dioxoisoindol-5- | | J = 7.4 Hz, 3H), 3.19 (t, J = 7.0 Hz, 2H), 2.89-2.64 |
| | yl]amino]octyl)benzamide | | (m, 3H), 2.14-2.02 (m, 1H), 1.72-1.58 (m, 4H), |
| | | | 1.55-1.28 (m, 9H) |
| 105 | (2S,4S)-1-((S)-2-(10-(4-(2- | 467.55 | 1H NMR (300 MHz, DMSO-d6) δ 14.37 (s, 1H), |
| | ((3-amino-6-(2- | (M + 2)²⁺ | 8.99 (s, 1H), 8.63 (t, J = 6.1 Hz, 1H), 8.38 (t, J = |
| | hydroxyphenyl)pyridazin-4- | | 5.6 Hz, 1H), 7.94 (dd, J = 8.5, 1.6 Hz, 1H), 7.82 |
| | yl)oxy)ethyl)benzamido)decanamido)- | | (dd, J = 12.9, 8.5 Hz, 3H), 7.61 (s, 1H), 7.49 (d, |
| | 3,3-dimethylbutanoyl)-4- | | J = 8.1 Hz, 2H), 7.46-7.32 (m, 4H), 7.24 (td, J = |
| | hydroxy-N-(4-(4- | | 7.5, 1.5 Hz, 1H), 6.93-6.78 (m, 2H), 6.52 (s, 2H), |
| | methylthiazol-5- | | 5.44 (d, J = 7.2 Hz, 1H), 4.54-4.40 (m, 4H), 4.36 |
| | yl)benzyl)pyrrolidine-2- | | (dd, J = 8.5, 6.2 Hz, 1H), 4.28 (d, J = 5.5 Hz, 1H), |
| | carboxamide | | 4.26-4.14 (m, 1H), 3.94 (dd, J = 10.1, 5.6 Hz, |
| | | | 1H), 3.44 (dd, J = 10.1, 5.3 Hz, 1H), 3.33 (s, 4H), |
| | | | 3.22 (p, J = 6.7 Hz, 3H), 2.45 (s, 3H), 2.40-2.10 |
| | | | (m, 1H), 1.74 (dt, J = 12.4, 6.1 Hz, 1H), 1.49 (d, |
| | | | J = 11.1 Hz, 5H), 1.25 (s, 12H), 0.95 (s, 9H). |
| 149 | 4-(2-((3-amino-6-(2- | 718.9 | 1H NMR (400 MHz, DMSO-d6) δ 14.35 (s, 1H), |
| | hydroxyphenyl)pyridazin-4- | | 11.09 (s, 1H), 8.40 (t, J = 5.6 Hz, 1H), 7.99-7.91 |
| | yl)oxy)ethyl)-N-(2-(4-(2-(2,6- | | (m, 1H), 7.81 (d, J = 8.1 Hz, 2H), 7.70 (dd, J = |
| | dioxopiperidin-3-yl)-1,3- | | 8.4, 7.2 Hz, 1H), 7.61 (s, 1H), 7.51 (d, J = 8.1 Hz, |
| | dioxoisoindolin-4- | | 2H), 7.35 (t, J = 7.3 Hz, 2H), 7.23 (td, J = 7.6, 1.6 |
| | yl)piperazin-1- | | Hz, 1H), 6.93-6.83 (m, 2H), 6.51 (s, 2H), 5.09 |
| | yl)ethyl)benzamide | | (dd, J = 12.9, 5.4 Hz, 1H), 4.49 (t, J = 6.7 Hz, 2H), |
| | | | 3.43 (d, J = 6.6 Hz, 2H), 3.30 (s, 4H), 3.20 (t, J = |
| | | | 6.7 Hz, 2H), 2.94-2.81 (m, 1H), 2.62 (q, J = 4.9 |
| | | | Hz, 4H), 2.55 (t, J = 7.0 Hz, 4H), 2.08-1.96 (m, |
| | | | 1H). |
| 161 | 4-(2-((3-amino-6-(2- | 704.3 | 1H NMR (400 MHz, DMSO-d6) δ 14.37 (s, 1H), |
| | hydroxyphenyl)pyridazin-4- | | 11.07 (s, 1H), 8.43 (t, J = 5.6 Hz, 1H), 7.94 (d, J = |
| | yl)oxy)ethyl)-N-(3-(1-(2-(2,6- | | 7.6 Hz, 1H), 7.81 (d, J = 7.6 Hz, 2H), 7.61-7.48 |
| | dioxopiperidin-3-yl)-1,3- | | (m, 4H), 7.27-7.20 (m, 1H), 7.09 (d, J = 7.2 Hz, |
| | dioxoisoindolin-4-yl)azetidin- | | 1H), 6.89-6.85 (m, 2H), 6.76 (d, J = 8.4 Hz, 1H), |
| | 3-yl)propyl)benzamide | | 6.52 (s, 2H), 5.04 (dd, J = 12.8, 5.2 Hz, 1H), 4.48 |
| | | | (t, J = 6.8 Hz, 2H), 4.30 (t, J = 6.8 Hz, 2H), 3.80 (t, |
| | | | J = 6.8 Hz, 2H), 3.30-3.20 (m, 2H), 3.19 (t, J = |
| | | | 6.8 Hz, 2H), 2.90-2.80 (m, 2H), 2.78-2.70 (m, |
| | | | 2H), 2.06-1.92 (m, 1H), 1.70-1.59 (m, 2H), 1.58- |
| | | | 1.49 (m, 2H). |
| 162 | 14-(4-(2-((3-amino-6-(2- | 1019.2 | 1H NMR (400 MHz, DMSO-d6) δ 14.30 (s, 1H), |
| | hydroxyphenyl)pyridazin-4- | | 8.98 (s, 1H), 8.60 (t, J = 6.1 Hz, 1H), 8.46 (t, J = |
| | yl)oxy)ethyl)benzamido)-N- | | 5.5 Hz, 1H), 8.00-7.90 (m, 1H), 7.81 (d, J = 8.1 |
| | ((S)-1-((2S,4R)-4-hydroxy-2- | | Hz, 2H), 7.61 (s, 1H), 7.49 (d, J = 8.0 Hz, 2H), |
| | (((4-(4-methylthiazol-5- | | 7.40 (d, J = 5.9 Hz, 5H), 7.23 (td, J = 7.6, 1.6 Hz, |
| | yl)benzyl)amino)methyl)pyrrolidin- | | 1H), 6.87 (t, J = 7.5 Hz, 2H), 6.53 (d, J = 3.5 Hz, |
| | 1-yl)-3,3-dimethyl-1- | | 2H), 5.16 (d, J = 3.5 Hz, 1H), 4.61-4.31 (m, 6H), |
| | oxobutan-2-yl)-3,6,9,12- | | 4.25 (dd, J = 15.8, 5.6 Hz, 1H), 3.95 (s, 2H), 3.67 |
| | tetraoxatetradecanamide | | (dd, J = 10.7, 3.9 Hz, 1H), 3.62-3.54 (m, 5H), |
| | | | 3.51 (dd, J = 8.8, 5.0 Hz, 10H), 3.39 (q, J = 8.1, |
| | | | 6.8 Hz, 2H), 3.20 (t, J = 6.7 Hz, 2H), 2.44 (s, 3H), |
| | | | 2.06 (dd, J = 14.8, 6.0 Hz, 1H), 1.90 (ddd, J = |
| | | | 13.0, 8.8, 4.4 Hz, 1H), 0.94 (s, 9H). |
| 167 | (2S,4R)-1-((S)-1-(4-(2-((3- | 1041.6 | 1H NMR (400 MHz, DMSO-d6) δ 14.34 (s, 1H), |
| | amino-6-(2- | | 8.98 (d, J = 2.5 Hz, 1H), 8.59 (t, J = 6.1 Hz, 1H), |
| | hydroxyphenyl)pyridazin-4- | | 8.46 (t, J = 5.6 Hz, 1H), 7.91 (d, J = 8.0 Hz, 1H), |
| | yl)oxy)ethyl)phenyl)-21-(tert- | | 7.84-7.78 (m, 2H), 7.61 (s, 1H), 7.49 (d, J = 8.3 |
| | butyl)-1,19-dioxo- | | Hz, 2H), 7.42 (d, J = 9.9 Hz, 5H), 7.24 (t, J = 7.7 |
| | 5,8,11,14,17-pentaoxa-2,20- | | Hz, 1H), 6.88 (t, J = 7.9 Hz, 2H), 6.59 (s, 2H), δ |
| | diazadocosan-22-oyl)-4- | | 5.15 (s, 1H), 4.56 (d, J = 9.6 Hz, 1H), 4.53-4.24 |
| | hydroxy-N-(4-(4- | | (m, 5H), 4.23 (d, J = 5.5 Hz, 1H), 3.96 (s, 2H), |
| | methylthiazol-5- | | 3.67 (dd, J = 10.6, 3.8 Hz, 1H), 3.64-3.58 (m, 3H), |
| | yl)benzyl)pyrrolidine-2- | | 3.55 (d, J = 6.3 Hz, 3H), 3.51 (m, 10H), 3.46 (m, |
| | carboxamide | | 4H), 3.40 (d, J = 6.0 Hz, 2H), 3.20 (t, J = 6.7 Hz, |
| | | | 2H), 2.44 (s, 2H), 2.05 (t, J = 10.4 Hz, 1H), 1.96- |
| | | | 1.77 (m, 1H), 0.94 (s, 9H). |
| 163 | (2S,4R)-1-((S)-2-(2-(2-(4-(2- | 865.4 | 1H NMR (300 MHz, DMSO-d6) δ 14.44 (s, 1H), |
| | ((3-amino-6-(2- | | 9.05 (s, 1H), 8.66 (s, 2H), 7.96 (dd, J = 31.3, 7.8 |
| | hydroxyphenyl)pyridazin-4- | | Hz, 3H), 7.74-7.39 (m, 8H), 7.29 (d, J = 8.0 Hz, |
| | yl)oxy)ethyl)benzamido)eth- | | 1H), 6.95 (d, J = 7.9 Hz, 2H), 6.58 (s, 2H), 5.24 (s, |
| | oxy)acetamido)-3,3- | | 1H), 4.69-4.23 (m, 6H), 4.07 (s, 2H), 3.70 (s, |
| | dimethylbutanoyl)-4- | | 4H), 3.53 (s, 1H), 3.26 (s, 4H), 2.51 (s, 3H), 2.27- |
| | hydroxy-N-(4-(4- | | 1.88 (m, 2H), 0.98 (s, 9H). |
| | methylthiazol-5- | | |
| | yl)benzyl)pyrrolidine-2- | | |
| | carboxamide | | |
| 170 | (2S,4R)-1-((10-(4-(2-((3- | 919.5 | 1H NMR (400 MHz, DMSO-d6) δ 14.36 (s, 1H), |
| | amino-6-(2- | | 8.98 (s, 1H), 8.48 (t, J = 6.0 Hz, 1H), 8.38 (t, J = |
| | hydroxyphenyl)pyridazin-4- | | 5.6 Hz, 1H), 7.93 (t, J = 7.5 Hz, 2H), 7.80 (d, J = |
| | yl)oxy)ethyl)benzamido)decanoyl)- | | 8.1 Hz, 2H), 7.61 (s, 1H), 7.49 (d, J = 8.0 Hz, 2H), |
| | L-valyl)-4-hydroxy-N- | | 7.39 (q, J = 8.3 Hz, 4H), 7.28-7.19 (m, 1H), 6.89 |
| | (4-(4-methylthiazol-5- | | (d, J = 7.8 Hz, 2H), 6.55 (s, 2H), 5.11 (s, 1H), 4.49 |
| | yl)benzyl)pyrrolidine-2- | | (t, J = 6.8 Hz, 2H), 4.43-4.22 (m, 5H), 3.65 (d, J = |
| | carboxamide | | 4.0 Hz, 2H), 3.21 (dd, J = 16.1, 6.6 Hz, 4H), 2.45 |
| | | | (s, 3H), 2.23-1.80 (m, 5H), 1.49 (m, 4H), 1.24 (s, |
| | | | 10H), 0.85 (dd, J = 17.5, 6.6 Hz, 6H). |
| 171 | (2S,4R)-1-((S)-2-(10-(4-(2- | 946.9 | 1H NMR (400 MHz, DMSO-d6) δ 14.36 (s, 1H), |
| | ((3-amino-6-(2- | | 8.98 (s, 1H), 8.38 (t, J = 7.0 Hz, 2H), 7.97-7.90 |
| | hydroxyphenyl)pyridazin-4- | | (m, 1H), 7.79 (dd, J = 9.1, 7.5 Hz, 3H), 7.61 (s, |
| | yl)oxy)ethyl)benzamido)decanamido)- | | 1H), 7.52-7.49 (m, 2H), 7.47-7.44 (m, 2H), |
| | 3,3-dimethylbutanoyl)-4- | | 7.42-7.38 (m, 2H), 7.23 (td, J = 7.6, 1.6 Hz, 1H), |
| | hydroxy-N-((S)-1-(4-(4- | | 6.91-6.83 (m, 2H), 6.51 (s, 2H), 5.10 (d, J = 3.5 |
| | methylthiazol-5- | | Hz, 1H), 4.90 (q, J = 7.1 Hz, 1H), 4.55-4.45 (m, |
| | yl)phenyl)ethyl)pyrrolidine-2- | | 4H), 4.42 (t, J = 8.0 Hz, 1H), 4.27 (s, 1H), 3.60 (s, |
| | carboxamide | | 2H), 3.19 (t, J = 6.4 Hz, 4H), 2.45 (s, 3H), 2.24 (dt, |
| | | | J = 14.7, 7.6 Hz, 1H), 2.09 (dt, J = 14.2, 7.1 Hz, |
| | | | 1H), 2.01 (t, J = 10.3 Hz, 1H), 1.79 (ddd, J = 12.8, |
| | | | 8.5, 4.7 Hz, 1H), 1.48 (s, 4H), 1.42-1.37 (m, |
| | | | 3H), 1.36-1.25 (m, 10H), 0.92 (s, 9H). |

The following compounds in Table C2 were prepared using procedures similar to those used for the preparation of compound 21.

TABLE C2

| | | LCMS | |
|---|---|---|---|
| | | (ESI) | |
| No. | Name | m/z | ¹H NMR |
| 6 | 15-[[4-(2-[[3-amino-6-(2- | 908.4 | ¹H NMR (400 MHz, DMSO-d6) δ 11.02 (s, 1H), |
| | hydroxyphenyl)pyridazin-4- | | 9.99 (s, 1H), 8.36 (dt, J = 21.6, 5.8 Hz, 2H), 7.91 |
| | yl]oxy]ethyl)phenyl]formamido]- | | (d, J = 7.9 Hz, 1H), 7.85-7.74 (m, 2H), 7.74- |
| | N-([4-[(3-chloro-1H-indol- | | 7.65 (m, 2H), 7.62 (s, 1H), 7.48 (t, J = 5.3 Hz, 3H), |
| | 7-yl)sulfamoyl]phenyl]methyl) | | 7.34 (d, J = 8.0 Hz, 2H), 7.29-7.18 (m, 2H), 6.99- |
| | pentadecanamide | | 6.85 (m, 3H), 6.81 (d, J = 7.6 Hz, 1H), 6.62 (s, |
| | | | 2H), 4.50 (t, J = 6.7 Hz, 2H), 4.27 (d, J = 5.9 Hz, |
| | | | 2H), 3.21 (d, J = 8.6 Hz, 5H), 2.11 (t, J = 7.4 Hz, |
| | | | 2H), 1.49 (d, J = 8.0 Hz, 4H), 1.34-1.13 (m, 20H) |
| 11 | 11-[[4-(2-[[3-amino-6-(2- | 852.3 | ¹H NMR (400 MHz, DMSO-d6) δ 14.36 (s, 1H), |
| | hydroxyphenyl)pyridazin-4- | | 11.03 (s, 1H), 8.36 (dt, J = 24.0, 6.1 Hz, 2H), 7.94 |
| | yl]oxy]ethyl)phenyl]formamido]- | | (d, J = 7.8 Hz, 1H), 7.80 (d, J = 7.8 Hz, 2H), 7.69 |
| | N-([4-[(3-chloro-1H-indol- | | (d, J = 8.0 Hz, 2H), 7.61 (s, 1H), 7.57-7.42 (m, |
| | 7-yl)sulfamoyl]phenyl]methyl) | | 3H), 7.34 (d, J = 8.1 Hz, 2H), 7.22 (d, J = 8.1 Hz, |
| | undecanamide | | 2H), 7.00-6.75 (m, 4H), 6.53 (s, 2H), 4.49 (t, J = |
| | | | 6.8 Hz, 2H), 4.27 (d, J = 5.7 Hz, 2H), 3.22 (dq, J = |
| | | | 13.1, 7.0, 6.3 Hz, 3H), 2.11 (t, J = 7.4 Hz, 2H), |
| | | | 1.63-1.25 (m, 18H). |
| 12 | 10-[[4-(2-[[3-amino-6-(2- | 838.5 | ¹H NMR (400 MHz, DMSO-d6) δ 14.38 (brs, 1H), |
| | hydroxyphenyl)pyridazin-4- | | 11.05 (s, 1H), 8.39 (t, J = 5.0 Hz, 1H), 8.33 (t, J = |
| | yl]oxy]ethyl)phenyl]formamido]- | | 5.6 Hz, 1H), 7.95 (d, J = 7.6Hz, 1H), 7.80 (d, J = |
| | N-([4-[(3-chloro-1H-indol- | | 7.9 Hz, 2H), 7.69 (d, J = 8.0Hz, 2H), 7.61 (s, 1H), |
| | 7-yl)sulfamoyl]phenyl]methyl) | | 7.49 (d, J = 7.8 Hz, 2H), 7.45 (s, 1H), 7.33 (d, J = |
| | decanamide | | 8.0 Hz, 2H), 7.24 (t, J = 7.5Hz, 1H), 7.18 (d, J = |
| | | | 7.9 Hz, 1H), 6.96-6.84 (m, 2H), 6.81 (d, J = 7.5 |
| | | | Hz, 1H), 6.52 (s, 2H), 4.49 (t, J = 6.3 Hz, 2H), 4.27 |
| | | | (d, J = 5.3 Hz, 2H), 3.26-3.18 (m, 4H), 2.11 (t, J = |
| | | | 7.1 Hz, 2H), 1.53-1.47 (m, 4H), 1.36-1.15 (m, |
| | | | 12H) |
| 13 | 7-[[4-(2-[[3-amino-6-(2- | 796.75 | ¹H NMR (300 MHz, DMSO-d6) δ 14.26 (s, 1H), |
| | hydroxyphenyl)pyridazin-4- | | 11.04 (s, 1H), 10.00 (s, 1H), 8.42-8.33 (m, 2H), |
| | yl]oxy]ethyl)phenyl]formamido]- | | 7.92 (d, J = 7.9 Hz, 1H), 7.84-7.77 (m, 2H), 7.74- |
| | N-([4-[(3-chloro-1H-indol- | | 7.67 (m, 2H), 7.62 (s, 1H), 7.54-7.45 (m, 3H), |
| | 7-yl)sulfamoyl]phenyl]methyl) | | 7.38-7.32 (m, 2H), 7.30-7.19 (m, 2H), 6.99- |
| | heptanamide | | 6.78 (m, 4H), 6.62 (s, 1H), 4.50 (t, J = 6.7 Hz, 2H), |
| | | | 4.28 (d, J = 5.7 Hz, 2H), 3.21 (q, J = 6.4 Hz, 5H), |
| | | | 2.13 (t, J = 7.4 Hz, 2H), 1.50(s, 4H), 1.40-1.18 |
| | | | (m, 4H) |
| 14 | 6-[[4-(2-[[3-amino-6-(2- | 782.1 | ¹H NMR (400 MHz, DMSO-d6) δ 14.40 (brs, 1H), |
| | hydroxyphenyl)pyridazin-4- | | 11.03 (s, 1H), 8.40 (t, J = 5.4 Hz, 1H), 8.34 (t, J = |
| | yl]oxy]ethyl)phenyl]formamido]- | | 5.8 Hz, 1H), 7.95 (d, J = 7.5 Hz, 1H), 7.80 (d, J = |
| | N-([4-[(3-chloro-1H-indol- | | 8.1 Hz, 2H), 7.70 (d, J = 8.2 Hz, 2H), 7.61 (s, 1H), |
| | 7-yl)sulfamoyl]phenyl]methyl) | | 7.52-7.44 (m, 3H), 7.34 (d, J = 8.2 Hz, 2H), 7.23 |
| | hexanamide | | (dd, J = 18.4, 7.4 Hz, 2H), 6.97-6.78 (m, 4H), |
| | | | 6.52 (s, 2H), 4.49 (t, J = 6.6 Hz, 2H), 4.27 (d, J = |
| | | | 5.6 Hz, 2H), 3.28-3.16 (m, 5H), 2.14 (t, J = 7.3 |
| | | | Hz, 2H), 1.58-1.47 (m, 4H), 1.32-1.22 (m, 2H) |
| 15 | 4-(2-((3-amino-6-(2- | 768.35 | ¹H NMR (300 MHz, nDMSO-d6) δ 14.0 (s, 1H), |
| | hydroxyphenyl)pyridazin-4- | | 11.04 (s, 1H), 10.00 (s, 1H), 8.39 (dt, J = 17.5, 5.7 |
| | yl)oxy)ethyl)-N-(5-((4-(N-(3- | | Hz, 2H), 7.89 (d, J = 7.9 Hz, 1H), 7.84-7.78 (m, |
| | chloro-1H-indol-7- | | 2H), 7.70 (d, J = 8.3 Hz, 2H), 7.63 (s, 1H), 7.55- |
| | yl)sulfamoyl)benzyl)amino)- | | 7.44 (m, 3H), 7.35 (d, J = 8.3 Hz, 2H), 7.31-7.20 |
| | 5-oxopentyl)benzamide | | (m, 2H), 6.92 (dt, J = 11.9, 7.7 Hz, 3H), 6.81 (d, J = |
| | | | 7.7 Hz, 1H), 6.73 (s, 1H), 4.51 (t, J = 6.7 Hz, |
| | | | 2H), 4.28 (d, J = 5.6 Hz, 2H), 3.22 (q, J = 6.7 Hz, |
| | | | 5H), 2.17 (t, J = 6.8 Hz, 2H), 1.54 (t, J = 6.7 Hz, |
| | | | 4H) |
| 16 | 4-[[4-(2-[[3-amino-6-(2- | 754.1 | ¹H NMR (300 MHz, DMSO-d6) δ 14.00 (s, 1H), |
| | hydroxyphenyl)pyridazin-4- | | 11.04 (s, 1H), 10.00 (d, J = 2.6 Hz, 1H), 8.42 (dt, |
| | yl]oxy]ethyl)phenyl]formamido]- | | J = 12.0, 5.8 Hz, 2H), 7.91 (d, J = 7.9 Hz, 1H), |
| | N-([4-[(3-chloro-1H-indol- | | 7.84-7.77 (m, 2H), 7.70 (d, J = 8.1 Hz, 2H), 7.63 (s, |
| | 7-yl)sulfamoyl]phenyl]methyl) | | 1H), 7.54-7.44 (m, 3H), 7.36 (d, J = 8.1 Hz, 2H), |
| | butanamide | | 7.32-7.18 (m, 2H), 7.02-6.85 (m, 3H), 6.82 (d, |
| | | | J = 7.6 Hz, 1H), 6.66 (s, 1H), 4.50 (t, J = 6.7 Hz, |
| | | | 2H), 4.29 (d, J = 5.6 Hz, 2H), 3.23 (dt, J = 12.9, |
| | | | 6.3 Hz, 5H), 2.20 (t, J = 7.5 Hz, 2H), 1.77 (q, J = |
| | | | 7.3 Hz, 2H) |
| 17 | 3-[[4-(2-[[3-amino-6-(2- | 740.2 | ¹H NMR (400 MHz, DMSO-d6) δ 14.38 (s, 1H), |
| | hydroxyphenyl)pyridazin-4- | | 11.02 (s, 1H), 9.99 (s, 1H), 8.59-8.42 (m, 2H), |
| | yl]oxy]ethyl)phenyl]formamido]- | | 7.95 (d, J = 7.5 Hz, 1H), 7.81-7.75 (m, 2H), 7.69- |
| | N-([4-[(3-chloro-1H-indol- | | 7.60 (m, 3H), 7.50-7.44 (m, 3H), 7.39-7.30 |
| | 7-yl)sulfamoyl]phenyl]methyl) | | (m, 2H), 7.28-7.19 (m, 2H), 7.00-6.74 (m, 4H), |
| | propanamide | | 6.53 (s, 1H), 4.49 (t, J = 6.8 Hz, 2H), 4.30 (d, J = |
| | | | 5.8 Hz, 2H), 3.55-3.45 (m, 2H), 3.19 (t, J = 6.7 |
| | | | Hz, 2H), 2.59-2.41 (m, 3H) |
| 22 | 12-[[4-(2-[[3-amino-6-(2- | 866.35 | ¹H NMR (300 MHz, DMSO-d6) δ 11.03 (d, J = 2.8 |
| | hydroxyphenyl)pyridazin-4- | | Hz, 1H), 10.00 (s, 1H), 8.36 (dt, J = 17.0, 5.8 Hz, |
| | yl]oxy]ethyl)phenyl]formamido]- | | 2H), 7.80 (d, J = 8.2 Hz, 2H), 7.71-7.61 (m, 4H), |
| | N-([4-[(3-chloro-1H-indol- | | 7.54-7.28 (m, 6H), 7.24 (d, J = 7.7 Hz, 2H), 7.03- |
| | 7-yl)sulfamoyl]phenyl]methyl) | | 6.69 (m, 4H), 4.56 (t, J = 6.6 Hz, 2H), 4.27 (d, J = |
| | dodecanamide | | 5.7 Hz, 2H), 3.22 (d, J = 6.0 Hz, 6H), 2.11 (t, J = |
| | | | 7.4 Hz, 2H), 1.49 (s, 4H), 1.27 (d, J = 7.2 Hz, 4H), |
| | | | 1.23 (s, 10H) |
| 23 | 9-[[4-(2-[[3-amino-6-(2- | 824.1 | ¹H NMR (300 MHz, DMSO-d6) δ 11.03 (s, 1H), |
| | hydroxyphenyl)pyridazin-4- | | 10.10 (s, 1H), 8.37 (d, J = 19.9 Hz, 2H), 8.18- |
| | yl]oxy]ethyl)phenyl]formamido]- | | 7.57 (m, 7H), 7.56-7.11 (m, 7H), 6.86 (dt, J = |
| | N-([4-[(3-chloro-1H-indol- | | 23.3, 8.5 Hz, 4H), 6.54 (s, 2H), 4.49 (s, 2H), 4.27 |
| | 7-yl)sulfamoyl]phenyl]methyl) | | (d, J = 5.7 Hz, 2H), 3.28-3.04 (m, 4H), 2.12 (t, J = |
| | nonanamide | | 7.5 Hz, 2H), 1.50 (s, 4H), 1.26 (s, 8H) |
| 25 | 8-[[4-(2-[[3-amino-6-(2- | 810.2 | ¹H NMR (400 MHz, DMSO-d6) δ 11.02 (d, J = 2.8 |
| | hydroxyphenyl)pyridazin-4- | | Hz, 1H), 9.99 (s, 1H), 8.36 (dt, J = 24.0, 5.8 Hz, |
| | yl]oxy]ethyl)phenyl]formamido]- | | 2H), 7.91 (d, J = 7.9 Hz, 1H), 7.81 (d, J = 8.2 Hz, |
| | N-([4-[(3-chloro-1H-indol- | | 2H), 7.70 (d, J = 8.2 Hz, 2H), 7.62 (s, 1H), 7.56- |
| | 7-yl)sulfamoyl]phenyl]methyl) | | 7.45 (m, 3H), 7.35 (d, J = 8.2 Hz, 2H), 7.29-7.13 |
| | octanamide | | (m, 2H), 6.92 (dt, J = 21.8, 8.1 Hz, 3H), 6.82 (d, J = |
| | | | 7.6 Hz, 1H), 6.63 (s, 2H), 4.50 (t, J = 6.7 Hz, |
| | | | 2H), 4.27 (d, J = 5.8 Hz, 2H), 3.22 (dt, J = 17.1, |
| | | | 6.6 Hz, 4H), 2.12 (t, J = 7.4 Hz, 2H), 1.50 (p, J = |
| | | | 7.3 Hz, 4H), 1.26 (dq, J = 16.5, 7.6, 7.1 Hz, 7H) |

### Example 4. Preparation of N-(4-(2-((3-amino-6-(2-hydroxyphenyl)pyridazin-4-yl)oxy)ethyl)benzyl)-10-((2-(2,6-dioxopiperidin-3-yl)-1,3-dioxoisoindolin-5-yl)amino)decanamide (compound 75)

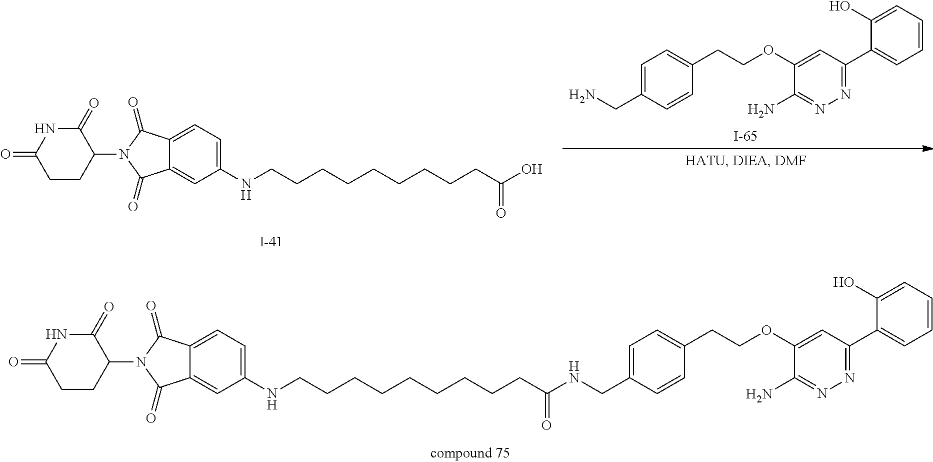

To a solution of 10-((2-(2,6-dioxopiperidin-3-yl)-1,3-dioxoisoindolin-5-yl)amino)decanoic acid (I-41, 25.00 mg, 0.056 mmol, 1.00 equiv) in DMF (1 mL) was added HATU (25.72 mg, 0.067 mmol, 1.20 equiv) and DIEA (21.81 mg, 0.169 mmol, 3.00 equiv) in portions at room temperature under an atmosphere of dry nitrogen. The resulting mixture was stirred for 30 min. To the above mixture was added 2-(6-amino-5-(4-(aminomethyl)phenethoxy)pyridazin-3-yl)phenol (I-65, 18.96 mg, 0.056 mmol, 1.00 equiv) in portions at room temperature. The resulting mixture was stirred for an additional hour then quenched with water (3 mL). The resulting mixture was extracted with EtOAc (3×3 mL). The combined organic layers were washed with brine (10 mL), dried over anhydrous Na₂SO₄, filtered, and concentrated under reduced pressure. The crude product was purified by reversed-phase preparative HPLC to afford compound 75 (14.6 mg, 34.00%) as a white solid. ¹H NMR (300 MHz, Methanol-d₄) δ 7.76 (dd, J=8.2, 1.6 Hz, 1H), 7.51 (d, J=8.2 Hz, 1H), 7.43 (s, 1H), 7.34 (d, J=8.1 Hz, 2H), 7.31-7.18 (m, 3H), 6.99 (d, J=2.1 Hz, 1H), 6.97-6.79 (m, 3H), 5.05 (dd, J=12.7, 5.5 Hz, 1H), 4.61 (s, 2H), 4.47 (t, J=6.6 Hz, 2H), 4.34 (s, 2H), 3.76 (dt, J=8.4, 3.8 Hz, 2H), 3.20 (t, J=6.6 Hz, 2H), 2.91-2.77 (m, 2H), 2.64 (dd, J=12.3, 6.8 Hz, 1H), 2.23 (t, J=7.3 Hz, 2H), 2.06 (q, J=6.7, 5.9 Hz, 1H), 1.58 (d, J=34.6 Hz, 2H), 1.30 (d, J=2.7 Hz, 10H). LCMS (ESI) m/z [M+H]⁺=762.55.

The following compounds in Table D1 were prepared using procedures similar to those used for the preparation of compound 75.

TABLE D1

| | | LCMS | |
|---|---|---|---|
| No. | Name | (ESI) m/z | ¹H NMR |
| 7 | N-[[4-(2-[[3-amino-6-(2- | 748.25 | ¹H NMR (400 MHz, DMSO-d6) δ 14.40 (s, 1H), |
| | hydroxyphenyl)pyridazin- | | 11.14 (s, 1H), 8.53 (s, 1H), 7.95 (s, 2H), 7.81 (s, |
| | 4-yl]oxy]ethyl)phenyl] | | 1H), 7.59 (s, 1H), 7.49 (d, J = 7.3 Hz, 1H), 7.41 (d, |
| | methyl]-2-[(2-[[2-(2,6- | | J = 8.5 Hz, 1H), 7.36-7.30 (m, 2H), 7.27-7.17 |
| | dioxopiperidin-3-yl)-1,3- | | (m, 3H), 6.87 (t, J = 8.5 Hz, 2H), 6.53 (d, J = 15.3 |
| | dioxoisoindol-4-yl] | | Hz, 2H), 5.12 (dd, J = 13.3, 5.4 Hz, 1H), 4.77 (s, |
| | oxy]acetamido)methyl] | | 2H), 4.42 (t, J = 6.9 Hz, 2H), 4.26 (d, J = 5.7 Hz, |
| | cyclopropane-1- | | 2H), 3.10 (t, J = 6.7 Hz, 2H), 2.87 (d, J = 12.5 Hz, |
| | carboxamide | | 2H), 2.69-2.66 (m, 1H), 2.61 (s, 2H), 2.33 (t, J = |
| | | | 1.9 Hz, 1H), 2.02 (d, J = 11.6 Hz, 1H), 1.73 (q, J = |
| | | | 7.6 Hz, 1H), 1.40-1.34 (m, 1H), 0.94-0.85 (m, |
| | | | 2H) |
| 19 | N-(4-(2-((3-amino-6-(2- | 747.95 | ¹H NMR (300 MHz, DMSO-d6) δ 11.13 (s, 1H), |
| | hydroxyphenyl)pyridazin- | | 8.32 (t, J = 5.9 Hz, 1H), 8.14 (s, 1H), 8.00-7.90 |
| | 4-yl)oxy)ethyl)benzyl)-1- | | (m, 1H), 7.85 (d, J = 8.3 Hz, 1H), 7.60 (s, 1H), |
| | (2-((2-(2,6-dioxopiperidin- | | 7.49 (d, J = 2.2 Hz, 1H), 7.37 (dd, J = 9.4, 7.5 Hz, |
| | 3-yl)-1,3-dioxoisoindolin- | | 3H), 7.29-7.14 (m, 3H), 6.93-6.81 (m, 2H), |
| | 5-yl)oxy)ethyl)piperidine- | | 6.52 (s, 2H), 5.13 (dd, J = 12.9, 5.4 Hz, 1H), 4.43 |
| | 4-carboxamide | | (t, J = 6.8 Hz, 2H), 4.35 (s, 2H), 4.24 (d, J = 5.8 |
| | | | Hz, 2H), 3.12 (t, J = 6.9 Hz, 4H), 2.88 (d, J = 13.4 |
| | | | Hz, 2H), 2.62-2.54 (m, 1H), 2.25-2.15 (m, 2H), |
| | | | 2.13-1.92 (m, 2H), 1.70-1.55 (m, 4H) |
| 27 | N′-[[4-(2-[[3-amino-6-(2- | 879 | ¹H NMR (400 MHz, Methanol-d4) δ 8.88 (s, 1H), |
| | hydroxyphenyl)pyridazin- | | 7.77 (d, J = 8.1 Hz, 1H), 7.49-7.37 (m, 5H), 7.35 |
| | 4-yl]oxy]ethyl)phenyl] | | (d, J = 8.1 Hz, 2H), 7.26 (dd, J = 7.6, 5.9 Hz, 3H), |
| | methyl]-N-[(2S)-1- | | 6.97-6.86 (m, 2H), 4.64 (d, J = 5.6 Hz, 1H), 4.61- |
| | [(2S,4R)-4-hydroxy-2-([[4- | | 4.54 (m, 1H), 4.54-4.44 (m, 4H), 4.36 (d, J = |
| | (4-methyl-1,3-thiazol-5- | | 15.7 Hz, 3H), 3.92 (d, J = 10.9 Hz, 1H), 3.80 (dd, |
| | yl)phenyl]methyl]carba- | | J = 11.0, 3.9 Hz, 1H), 3.26-3.15 (m, 2H), 2.48 (s, |
| | moyl)pyrrolidin-1-yl]-3,3- | | 3H), 2.37-2.15 (m, 5H), 2.15-1.99 (m, 1H), |
| | dimethyl-1-oxobutan-2- | | 1.65 (d, J = 4.0 Hz, 4H), 1.04 (s, 9H) |
| | yl]hexanediamide | | |
| 28 | N′-[[4-(2-[[3-amino-6-(2- | 947.3 | ¹H NMR (400 MHz, Methanol-d4) δ 8.88 (s, 1H), |
| | hydroxyphenyl)pyridazin- | | 7.79-7.74 (m, 1H), 7.50-7.39 (m, 5H), 7.35 (d, |
| | 4-yl]oxy]ethyl)phenyl] | | J = 8.0 Hz, 2H), 7.31-7.19 (m, 3H), 6.96-6.87 |
| | methyl]-N-[(2S)-1- | | (m, 2H), 4.66 (d, J = 5.7 Hz, 1H), 4.62-4.55 (m, |
| | [(2S,4R)-4-hydroxy-2-([[4- | | 2H), 4.50 (q, J = 6.7 Hz, 3H), 4.33 (s, 3H), 3.91 (d, |
| | (4-methyl-1,3-thiazol-5- | | J = 10.5 Hz, 1H), 3.81 (dd, J = 11.1,4.1 Hz, 1H), |
| | yl)phenyl]methyl]carba | | 3.21 (t, J = 6.5 Hz, 2H), 2.49 (d, J = 6.1 Hz, 3H), |
| | moyl)pyrrolidin-1-yl]-3,3- | | 2.25 (dt, J = 23.9, 7.7 Hz, 6H), 2.16-2.02 (m, |
| | dimethyl-1-oxobutan-2- | | 1H), 1.61 (s, 4H), 1.32 (d, J = 11.0 Hz, 13H), 1.05 |
| | yl]undecanediamide | | (d, J = 3.3 Hz, 9H) |
| 29 | N′-[[4-(2-[[3-amino-6-(2- | 849.2 | ¹H NMR (400 MHz, Methanol-d4) δ 8.88 (s, 1H), |
| | hydroxyphenyl)pyridazin- | | 7.77 (dd, J = 8.3, 1.6 Hz, 1H), 7.52-7.38 (m, 5H), |
| | 4-yl]oxy]ethyl)phenyl] | | 7.35 (d, J = 8.0 Hz, 2H), 7.31-7.20 (m, 3H), 6.92 |
| | methyl]-N-[(2S)-1- | | (dtd, J = 8.4, 3.7, 1.3 Hz, 2H), 4.68-4.54 (m, 2H), |
| | [(2S,4R)-4-hydroxy-2-([[4- | | 4.54-4.43 (m, 4H), 4.34 (s, 3H), 3.89 (d, J = 11.0 |
| | (4-methyl-1,3-thiazol-5- | | Hz, 1H), 3.79 (dd, J = 11.0, 3.9 Hz, 1H), 3.21 (t, J = |
| | yl)phenyl]methyl] | | 6.5 Hz, 2H), 2.71-2.58 (m, 4H), 2.58-2.50 |
| | carbamoyl)pyrrolidin-1-yl]- | | (m, 3H), 2.25-2.14 (m, 1H), 2.07 (ddd, J = 13.3, |
| | 3,3-dimethyl-1-oxobutan- | | 9.1,4.5 Hz, 1H), 1.04 (s, 9H) |
| | 2-yl]succinamide | | |
| 30 | N1-(4-(2-((3-amino-6-(2- | 905.55 | ¹H NMR (400 MHz, DMSO-d6) δ 14.37 (s, 1H), |
| | hydroxyphenyl)pyridazin- | | 8.99 (s, 1H), 8.55 (t, J = 6.0 Hz, 1H), 8.25 (t, J = |
| | 4-yl)oxy)ethyl)benzyl)- | | 5.9 Hz, 1H), 7.98-7.91 (m, 1H), 7.84 (d, J = 9.3 |
| | N15-((S)-1-((2S,4R)-4- | | Hz, 1H), 7.60 (s, 1H), 7.46-7.31 (m, 6H), 7.28- |
| | hydroxy-2-((4-(4- | | 7.16 (m, 3H), 6.87 (t, J = 7.6 Hz, 2H), 6.49 (s, 2H), |
| | methylthiazol-5- | | 5.12 (d, J = 3.6 Hz, 1H), 4.55 (d, J = 9.4 Hz, 1H), |
| | yl)benzyl)carbamoyl)pyrrol | | 4.47-4.38 (m, 4H), 4.35 (s, 1H), 4.22 (dd, J = |
| | idin-1-yl)-3,3-dimethyl-1- | | 11.8, 5.7 Hz, 3H), 3.66 (d, J = 4.3 Hz, 2H), 3.12 (t, |
| | oxobutan-2- | | J = 6.8 Hz, 2H), 2.45 (s, 3H), 2.25 (dt, J = 14.8, |
| | yl)pentadecanediamide | | 7.5 Hz, 1H), 2.11 (q, J = 6.7, 6.1 Hz, 3H), 2.03 (t, |
| | | | J = 10.4 Hz, 1H), 1.90-1.78 (m, 1H), 1.49-1.43 (m, |
| | | | 4H), 1.25-1.20 (m, 4H), 0.94 (s, 9H) |
| 31 | N1-(4-(2-((3-amino-6-(2- | 975.4 | ¹H NMR (400 MHz, DMSO-d6) δ 14.34 (brs, 1H), |
| | hydroxyphenyl)pyridazin- | | 8.98 (s, 1H), 8.58 (t, J = 6.0 Hz, 1H), 8.23 (t, J = |
| | 4-yl)oxy)ethyl)benzyl)- | | 6.0 Hz, 1H), 7.97-7.79 (m, 2H), 7.60 (s, 1H), |
| | N13-((S)-1-((2S,4R)-4- | | 7.47-7.31 (m, 6H), 7.28-7.15 (m, 3H), 6.92- |
| | hydroxy-2-((4-(4- | | 6.83 (m, 2H), 6.52 (s, 2H), 5.12 (d, J = 3.5 Hz, |
| | methylthiazol-5-yl)benzyl) | | 1H), 4.54 (d, J = 9.4 Hz, 1H), 4.43 (td, J = 7.1,4.5 |
| | carbamoyl)pyrrolidin- | | Hz, 4H), 4.35 (s, 1H), 4.22 (q, J = 5.0 Hz, 3H), |
| | 1-yl)-3,3-dimethyl-1- | | 3.72-3.60 (m, 2H), 3.12 (t, J = 6.8 Hz, 2H), 2.45 |
| | oxobutan-2-yl) | | (s, 3H), 2.26 (dt, J = 14.8, 7.7 Hz, 1H), 2.15-1.99 |
| | tridecanediamide | | (m, 4H), 1.91 (ddd, J = 12.9, 8.6, 4.6 Hz, 1H), |
| | | | 1.52-1.40 (m, 4H), 1.28-1.08 (m, 14H), 0.94 (s, |
| | | | 9H) |
| 32 | N1-(4-(2-((3-amino-6-(2- | | ¹H NMR (400 MHz, DMSO-d6) δ 14.36 (brs, 1H), |
| | hydroxyphenyl)pyridazin- | | 8.98 (s, 1H), 8.58 (t, J = 5.9 Hz, 1H), 8.28 (t, J = |
| | 4-yl)oxy)ethyl)benzyl)- | | 5.9 Hz, 1H), 7.98-7.79 (m, 2H), 7.60 (s, 1H), |
| | N17-((S)-1-((2S,4R)-4- | | 7.47-7.31 (m, 6H), 7.28-7.12 (m, 3H), 6.92- |
| | hydroxy-2-((4-(4- | | 6.83 (m, 2H), 6.50 (s, 2H), 5.12 (d, J = 3.6 Hz, |
| | methylthiazol-5-yl) | | 1H), 4.55 (d, J = 9.4 Hz, 1H), 4.49-4.39 (m, 4H), |
| | benzyl)carbamoyl)pyrrolidin- | | 4.35 (s, 1H), 4.22 (q, J = 5.4, 4.8 Hz, 3H), 3.72- |
| | 1-yl)-3,3-dimethyl-1- | | 3.60 (m, 2H), 3.12 (t, J = 6.9 Hz, 2H), 2.45 (s, 3H), |
| | oxobutan-2-yl) | | 2.26 (dt, J = 14.9, 7.7 Hz, 1H), 2.15-1.99 (m, |
| | heptadecanediamide | | 3H), 1.91-1.83 (m, 1H), 1.54-1.40 (m, 4H), 1.23- |
| | | | 1.03 (m, 22H), 0.94 (s, 9H) |
| 33 | N-[[4-(2-[[3-amino-6-(2- | 919.4 | ¹H NMR (300 MHz, Methanol-d4) δ 8.87 (s, 1H), |
| | hydroxyphenyl)pyridazin- | | 7.76 (dd, J = 8.3, 1.6 Hz, 1H), 7.53-7.37 (m, 5H), |
| | 4-yl]oxy]ethyl) | | 7.38-7.30 (m, 2H), 7.30-7.18 (m, 3H), 6.99- |
| | phenyl]methyl]-N- | | 6.87 (m, 2H), 4.69-4.54 (m, 6H), 4.39 (s, 1H), |
| | [(2S)-1-[(2S,4R)-4- | | 4.33 (d, J = 2.3 Hz, 2H), 3.98-3.74 (m, 2H), 3.21 |
| | hydroxy-2-([[4-(4-methyl- | | (t, J = 6.5 Hz, 2H), 2.48 (s, 3H), 2.39-2.16 (m, |
| | 1,3-thiazol-5-yl)phenyl] | | 5H), 2.18-2.00 (m, 1H), 1.74-1.52 (m, 4H), |
| | methyl]carbamoyl) | | 1.49-1.28 (s, 6H), 1.04 (s, 9H) |
| | pyrrolidin-1-yl]-3,3- | | |
| | dimethyl-1-oxobutan-2- | | |
| | yl]nonanediamide | | |
| 34 | N-[[4-(2-[[3-amino-6-(2- | 863.3 | ¹H NMR (300 MHz, Methanol-d4) δ 8.87 (s, 1H), |
| | hydroxyphenyl)pyridazin- | | 7.75 (dd, J = 8.3, 1.6 Hz, 1H), 7.49-7.34 (m, 5H), |
| | 4-yl]oxy]ethyl) | | 7.38-7.30 (m, 2H), 7.30-7.18 (m, 3H), 6.98- |
| | phenyl]methyl] | | 6.85 (m, 2H), 4.66-4.55 (m, 2H), 4.54-4.41 (m, |
| | -N-[(2S)-1-[(2S,4R)-4- | | 6H), 4.41-4.28 (m, 3H), 3.93 (d, J = 11.1 Hz, |
| | hydroxy-2-([[4-(4-methyl- | | 1H), 3.81 (dd, J = 11.0, 3.9 Hz, 1H), 3.24-3.12 |
| | 1,3-thiazol-5-yl)phenyl] | | (m, 2H), 2.50-2.42 (m, 3H), 2.38-2.17 (m, 5H), |
| | methyl]carbamoyl) | | 2.18-2.00 (m, 1H), 1.98 (m, 2H), 1.03(s, 9H) |
| | pyrrolidin-1-yl]-3,3- | | |
| | dimethyl-1-oxobutan-2- | | |
| | yl]pentanediamide | | |
| 35 | N1-(4-(2-((3-amino-6-(2- | 1003.65 | ¹H NMR (400 MHz, DMSO-d6) δ 8.98 (s, 1H), |
| | hydroxyphenyl)pyridazin- | | 8.55 (t, J = 6.1 Hz, 1H), 8.30-8.20 (m, 1H), 7.94 |
| | 4-yl)oxy)ethyl)benzyl)- | | (dd, J = 8.0, 1.6 Hz, 1H), 7.83 (d, J = 9.4 Hz, 1H), |
| | N15-((S)-1-((2S,4R)-4- | | 7.60 (s, 1H), 7.47-7.30 (m, 6H), 7.28-7.15 (m, |
| | hydroxy-2-((4-(4- | | 3H), 6.87 (t, J = 7.9 Hz, 2H), 6.49 (s, 2H), 5.11 (d, |
| | methylthiazol-5-yl)benzyl) | | J = 3.0 Hz, 1H), 4.54 (d, J = 9.4 Hz, 1H), 4.43 (td, |
| | carbamoyl)pyrrolidin-1- | | J = 7.2, 3.0 Hz, 4H), 4.35 (s, 1H), 4.22 (q, J = 5.4, |
| | yl)-3,3-dimethyl-1- | | 5.0 Hz, 3H), 3.77-3.57 (m, 2H), 3.12 (t, J = 6.9 |
| | oxobutan-2-yl) | | Hz, 2H), 2.45 (s, 3H), 2.26 (dt, J = 14.7, 7.6 Hz, |
| | pentadecanediamide | | 1H), 2.11 (t, J = 7.3 Hz, 4H), 1.95-1.85 (m, 1H), |
| | | | 1.60-1.48 (m, 4H), 1.23-1.03 (m, 18H), 0.94 (s, |
| | | | 9H) |
| 36 | N-[[4-(2-[[3-amino-6-(2- | 989.4 | ¹H NMR (400 MHz, Methanol-d4) δ 8.87 (s, 1H), |
| | hydroxyphenyl)pyridazin- | | 7.76 (dd, J = 8.2, 1.7 Hz, 1H), 7.52-7.39 (m, 5H), |
| | 4-yl]oxy]ethyl) | | 7.35 (d, J = 8.1 Hz, 2H), 6.99-6.85 (m, 2H), 4.69- |
| | phenyl]methyl]-N-[(2S)- | | 4.61 (m, 1H), 4.59 (td, J = 7.5, 3.0 Hz, 2H), 4.55- |
| | 1-[(2S,4R)-4-hydroxy-2- | | 4.43 (m, 7H), 4.36 (d, J = 17.8 Hz, 3H), 3.92 (d, |
| | ([[4-(4-methyl- | | J = 11.0 Hz, 1H), 3.81 (dd, J = 11.0, 3.9 Hz, 1H), |
| | 1,3-thiazol-5- | | 3.21 (t, J = 6.6 Hz, 2H), 2.56 (s, 3H), 2.35-2.16 |
| | yl)phenyl]methyl]carbamo | | (m, 4H), 2.09 (ddd, J = 18.3, 9.3, 4.8 Hz, 1H), 1.70- |
| | yl)pyrrolidin-1-yl]-3,3- | | 1.51 (m, 4H), 1.41-1.18 (m, 16H), 1.05 (s, 9H) |
| | dimethyl-1-oxobutan-2- | | |
| | yl]tetradecanediamide | | |
| 37 | N-[[4-(2-[[3-amino-6-(2- | 960.4 | ¹H NMR (400 MHz, Methanol-d4) δ 8.88 (s, 1H), |
| | hydroxyphenyl)pyridazin- | | 7.75 (dd, J = 8.3, 1.6 Hz, 1H), 7.45 (dt, J = 12.4, |
| | 4-yl]oxy]ethyl) | | 8.2 Hz, 5H), 7.35 (d, J = 7.8 Hz, 2H), 7.28 (td, J = |
| | phenyl]methyl]-N- | | 7.9, 1.9 Hz, 3H), 7.00-6.87 (m, 2H), 4.65 (d, J = |
| | [(2S)-1-[(2S,4R)-4- | | 3.3 Hz, 3H), 4.63-4.56 (m, 3H), 4.36 (d, J = 18.9 |
| | hydroxy-2-([[4-(4-methyl- | | Hz, 3H), 3.92 (d, J = 11.0 Hz, 1H), 3.81 (dd, J = |
| | 1,3-thiazol-5-yl)phenyl] | | 11.0, 3.9 Hz, 1H), 3.22 (t, J = 6.5 Hz, 2H), 2.48 |
| | methyl]carbamoyl) | | (s, 3H), 2.43-2.18 (m, 5H), 2.14-2.05 (m, 1H), |
| | pyrrolidin-1-yl]-3,3- | | 1.62 (d, J = 10.7 Hz, 4H), 1.42-1.23 (m, 12H), |
| | dimethyl-1-oxobutan-2-yl] | | 1.05 (s, 9H) |
| | dodecanediamide | | |
| 38 | (2S,4R)-1-[(2S)-2-[2-(2-[2- | 953.3 | ¹H NMR (400 MHz, Methanol-d4) δ 8.87 (s, 1H), |
| | [([[4-(2-[[3-amino-6-(2- | | 7.69 (dd, J = 8.2, 1.6 Hz, 1H), 7.53 (s, 1H), 7.50- |
| | hydroxyphenyl)pyridazin- | | 7.38 (m, 4H), 7.38-7.30 (m, 3H), 7.26 (d, J = 8.0 |
| | 4-yl]oxy]ethyl) | | Hz, 2H), 7.02-6.92 (m, 2H), 4.70 (s, 1H), 4.62- |
| | phenyl]methyl] | | 4.46 (m, 5H), 4.42 (d, J = 5.3 Hz, 2H), 4.39-4.29 |
| | carbamoyl)methoxy]ethox | | (m, 1H), 4.12-3.96 (m, 2H), 3.96-3.88 (m, 1H), |
| | y]ethoxy)acetamido]-3,3- | | 3.88-3.53 (m, 9H), 3.37 (s, 2H), 3.22 (t, J = 6.6 |
| | dimethylbutanoyl]-4-hy | | Hz, 2H), 2.47 (s, 3H), 2.22 (dd, J = 13.2, 7.7 Hz, |
| | droxy-N-[[4-(4-methyl- | | 1H), 2.15-2.03 (m, 1H), 1.03 (s, 9H) |
| | 1,3-thiazol-5-yl)phenyl] | | |
| | methyl]pyrrolidine- | | |
| | 2-carboxamide | | |
| 39 | N-[[4-(2-[[3-amino-6-(2- | 997.5 | ¹H NMR (400 MHz, Methanol-d4) δ 8.86 (s, 1H), |
| | hydroxyphenyl)pyridazin- | | 7.70 (dd, J = 8.2, 1.7 Hz, 1H), 7.52-7.21 (m, |
| | 4-yl]oxy]ethyl) | | 10H), 7.03-6.92 (m, 2H), 4.70 (s, 1H), 4.62- |
| | phenyl]methyl]-N- | | 4.47 (m, 6H), 4.40-4.30 (m, 3H), 4.03 (s, 2H), |
| | [(2S)-1-[(2S,4R)-4- | | 3.97 (d, J = 10.5 Hz, 2H), 3.88 (d, J = 11.0 Hz, |
| | hydroxy-2-([[4-(1,3- | | 1H), 3.80 (dd, J = 11.0, 3.8 Hz, 1H), 3.71-3.52 |
| | thiazol-5-yl)phenyl] | | (m, 11H), 3.22 (t, J = 6.4 Hz, 2H), 2.46 (s, 3H), |
| | methyl]carbamoyl) | | 2.28-2.20 (m, 1H), 2.17-2.03 (m, 1H), 1.03 (s, |
| | pyrrolidin-1-yl]-3,3- | | 9H) |
| | dimethyl-1-oxobutan- | | |
| | 2-yl]-3,6,9,12- | | |
| | tetraoxatetradecanediamide | | |
| 40 | N1-(4-(2-((3-amino-6-(2- | 1017.5 | ¹H NMR (300 MHz, DMSO-d6) δ 8.99 (s, 1H), |
| | hydroxyphenyl)pyridazin- | | 8.57 (t, J = 6.2 Hz, 1H), 8.27 (dd, J = 12.5, 6.4 Hz, |
| | 4-yl)oxy)ethyl)benzyl)- | | 1H), 7.95 (d, J = 7.9 Hz, 1H), 7.85 (d, J = 9.3 Hz, |
| | N16-((S)-1-((2S,4R)-4- | | 1H), 7.60 (s, 1H), 7.49-7.29 (m, 6H), 7.23-7.19 |
| | hydroxy-2-((4-(4- | | (m, 3H), 6.88 (d, J = 7.9 Hz, 2H), 6.51 (s, 2H), |
| | methylthiazol-5-yl) | | 5.15-5.10 (m, 1H), 4.55 (d, J = 9.4 Hz, 1H), 4.49- |
| | benzyl)carbamoyl)pyrrol | | 4.32 (m, 4H), 4.27-4.17 (m, 3H), 3.66 (d, J = |
| | idin-1-yl)-3,3-dimethyl- | | 4.6 Hz, 2H), 3.12 (t, J = 6.8 Hz, 2H), 2.45 (s, 3H), |
| | 1-oxobutan-2-yl) | | 2.25 (dd, J = 14.4, 7.0 Hz, 1H), 2.15-1.99 (m, |
| | hexadecanediamide | | 4H), 1.96-1.80 (m, 1H), 1.53-1.43 (m, 4H), |
| | | | 1.40-1.00 (m, 22H), 0.94 (s, 9H) |
| 62 | N-[[4-(2-[[3-amino-6-(2- | 651.25 | ¹H NMR (300 MHz, DMSO-d6) δ 14.33 (brs, 1H), |
| | hydroxyphenyl)pyridazin- | | 11.12 (s, 1H), 8.51 (t, J = 6.0 Hz, 1H), 7.93 (d, J = |
| | 4-yl]oxy]ethyl) | | 7.9 Hz, 1H), 7.81 (dd, J = 8.5, 7.3 Hz, 1H), 7.61 |
| | phenyl]methyl]-2- | | (s, 1H), 7.50 (d, J = 7.2 Hz, 1H), 7.46-7.32 (m, |
| | [[2-(2,6-dioxopiperidin- | | 3H), 7.31-7.17 (m, 3H), 6.88 (t, J = 7.5 Hz, 2H), |
| | 3-yl)-1,3-dioxoisoindol- | | 6.59 (brs, 2H), 5.12 (dd, J = 12.9, 5.4 Hz, 1H), |
| | 4-yl]oxy]acetamide | | 4.87 (s, 2H), 4.45 (t, J = 6.8 Hz, 2H), 4.38-4.23 |
| | | | (m, 2H), 3.13 (t, J = 6.8 Hz, 2H), 2.90 (ddd, J = |
| | | | 17.2, 13.9, 5.4 Hz, 1H), 2.80-2.50 (m, 2H), 2.12- |
| | | | 1.94 (m, 1H) |
| 63 | N-[[4-(2-[[3-amino-6-(2- | 707.45 | ¹H NMR (300 MHz, DMSO-d6) δ 14.38 (brs, 1H), |
| | hydroxyphenyl)pyridazin- | | 11.11 (s, 1H), 8.30 (q, J = 6.2 Hz, 1H), 8.01-7.90 |
| | 4-yl]oxy]ethyl) | | (m, 1H), 7.80 (dd, J = 8.5, 7.3 Hz, 1H), 7.59 (s, |
| | phenyl]methyl]-6- | | 1H), 7.51 (d, J = 8.5 Hz, 1H), 7.44 (d, J = 7.2 Hz, |
| | [[2-(2,6-dioxopiperidin- | | 1H), 7.35 (d, J = 8.0 Hz, 2H), 7.28-7.16 (m, 3H), |
| | 3-yl)-1,3-dioxoisoindol-4- | | 6.87 (t, J = 7.6 Hz, 2H), 6.51 (brs, 2H), 5.08 (dd, J = |
| | yl]oxy]hexanamide | | 12.9, 5.4 Hz, 1H), 4.43 (t, J = 6.8 Hz, 2H), 4.32- |
| | | | 4.08 (m, 4H), 3.11 (t, J = 6.8 Hz, 2H), 2.88 (ddd, |
| | | | J = 17.2, 13.9, 5.4 Hz, 1H), 2.79-2.57 (m, 2H), |
| | | | 2.17 (t, J = 7.3 Hz, 2H), 2.12-1.93 (m, 1H), 1.77 |
| | | | (p, J = 6.7 Hz, 2H), 1.61 (p, J = 7.2 Hz, 2H), 1.46 |
| | | | (q, J = 8.1 Hz, 2H) |
| 65 | N-[[4-(2-[[3-amino-6-(2- | 679.50 | ¹H NMR (300 MHz, DMSO-d6) δ 14.37 (brs, 1H), |
| | hydroxyphenyl)pyridazin- | | 11.11 (s, 1H), 8.37 (t, J = 5.9 Hz, 1H), 8.00-7.87 |
| | 4- | | (m, 1H), 7.81 (dd, J = 8.5, 7.2 Hz, 1H), 7.59 (s, |
| | yl]oxy]ethyl)phenyl]methyl] | | 1H), 7.51 (d, J = 8.6 Hz, 1H), 7.45 (d, J = 7.2 Hz, |
| | -4-[[2-(2,6-dioxopiperidin- | | 1H), 7.34 (d, J = 8.0 Hz, 2H), 7.30-7.13 (m, 3H), |
| | 3-yl)-1,3-dioxoisoindol-4- | | 6.97-6.80 (m, 2H), 6.50 (s, 2H), 5.08 (dd, J = |
| | yl]oxy]butanamide | | 12.9, 5.4 Hz, 1H), 4.43 (t, J = 6.8 Hz, 2H), 4.26 |
| | | | (m, 4H), 3.12 (t, J = 6.8 Hz, 2H), 2.88 (ddd, J = |
| | | | 17.3, 14.0, 5.5 Hz, 1H), 2.51 (m, 2H), 2.37 (t, J = |
| | | | 7.4 Hz, 2H), 2.03 (q, J = 6.9 Hz, 3H). |
| 66 | 4-(2-[[3-amino-6-(2- | 776.30 | ¹H NMR (300 MHz, Methanol-d4) δ 7.62-7.51 |
| | hydroxyphenyl)pyridazin- | | (m, 3H), 7.45 (ddd, J = 9.0, 7.6, 1.7 Hz, 1H), 7.34 |
| | 4-yl]oxy]ethyl)-N-[2-[2-(2- | | (d, J = 8.0 Hz, 2H), 7.26 (d, J = 8.0 Hz, 2H), 7.06 |
| | [[3-(2,6-dioxopiperidin-3- | | (t, J = 7.6 Hz, 2H), 6.96 (d, J = 2.1 Hz, 1H), 6.82 |
| | yl)-2-methyl-4- | | (dd, J = 8.4, 2.2 Hz, 1H), 5.04 (dd, J = 12.3, 5.4 |
| | oxoquinazolin-5-yl] | | Hz, 1H), 4.62 (t, J = 6.8 Hz, 2H), 4.34 (s, 2H), 3.22 |
| | amino]ethoxy)ethoxy] | | (dt, J = 13.8, 6.9 Hz, 4H), 2.98-2.64 (m, 3H), |
| | ethyl]benzamide | | 2.23 (t, J = 7.4 Hz, 2H), 2.09 (ddd, J = 11.4, 5.9, |
| | | | 3.2 Hz, 1H), 1.65 (q, J = 7.2 Hz, 3H), 1.33 (m, |
| | | | 16H) |
| 67 | N-[[4-(2-[[3-amino-6-(2- | 693.45 | ¹H NMR (300 MHz, Methanol-d4) δ 7.85-7.61 |
| | hydroxyphenyl)pyridazin- | | (m, 2H), 7.48 (s, 1H), 7.41 (dd, J = 7.9, 6.2 Hz, |
| | 4-yl]oxy]ethyl) | | 2H), 7.30 (q, J = 8.2 Hz, 5H), 6.95 (ddd, J = 8.3, |
| | phenyl]methyl]-5- | | 3.7, 1.9 Hz, 2H), 5.08 (dd, J = 12.6, 5.4 Hz, 1H), |
| | [[2-(2,6-dioxopiperidin- | | 4.51 (t, J = 6.6 Hz, 2H), 4.36 (s, 2H), 4.23 (d, J = |
| | 3-yl)-1,3-dioxoisoindol- | | 5.7 Hz, 2H), 3.21 (t, J = 6.6 Hz, 2H), 2.96-2.55 |
| | 4-yl]oxy]pentanamide | | (m, 3H), 2.49-2.30 (m, 2H), 2.09 (dtd, J = 11.6, |
| | | | 4.8, 4.0, 2.0 Hz, 1H), 1.89 (dt, J = 6.3, 3.4 Hz, 4H) |
| 68 | N-[[4-(2-[[3-amino-6-(2- | 721.35 | ¹H NMR (300 MHz, Methanol-d4) δ 7.72 (t, J = 7.6 |
| | hydroxyphenyl)pyridazin- | | Hz, 2H), 7.46 (s, 1H), 7.42-7.23 (m, 7H), 6.93 (d, |
| | 4-yl]oxy] | | J = 7.9 Hz, 2H), 5.08 (dd, J = 12.3, 5.4 Hz, 1H), |
| | ethyl)phenyl]methyl]-8- | | 4.51 (t, J = 6.6 Hz, 2H), 4.35 (d, J = 4.0 Hz, 2H), |
| | [[2-(2,6-dioxopiperidin- | | 4.14 (t, J = 6.4 Hz, 2H), 3.20 (t, J = 6.6 Hz, 2H), |
| | 3-yl)-1,3-dioxoisoindol- | | 2.92-2.63 (m, 3H), 2.26 (t, J = 7.2 Hz, 2H), 2.16 |
| | 4-yl]oxy]octanamide | | -2.00 (m, 1H), 1.73 (dq, J = 34.5, 7.3 Hz, 4H), |
| | | | 1.60-1.31 (m, 4H) |
| 69 | N-[[4-(2-[[3-amino-6-(2- | 735.30 | ¹H NMR (300 MHz, Methanol-d4) δ 7.70 (dd, J = |
| | hydroxyphenyl)pyridazin- | | 15.6, 7.7 Hz, 2H), 7.37 (dt, J = 12.6, 5.1 Hz, 4H), |
| | 4- | | 7.32-7.21 (m, 4H), 6.90 (t, J = 7.8 Hz, 2H), 5.09 |
| | yl]oxy]ethyl)phenyl]methyl] | | (dd, J = 12.2, 5.5 Hz, 1H), 4.47 (t, J = 6.6 Hz, 2H), |
| | -8-[[2-(2,6-dioxopiperidin- | | 4.34 (s, 2H), 4.14 (t, J = 6.4 Hz, 2H), 3.19 (t, J = |
| | 3-yl)-1,3-dioxoisoindol-4- | | 6.7 Hz, 2H), 2.78 (ddd, J = 20.0, 15.3, 10.6 Hz, |
| | yl]oxy]octanamide | | 3H), 2.25 (t, J = 7.1 Hz, 2H), 2.13 (s, 1H), 1.73 (dt, |
| | | | J = 40.8, 7.4 Hz, 4H), 1.44 (d, J = 37.9 Hz, 6H) |
| 70 | N-[[4-(2-[[3-amino-6-(2- | 752.45 | ¹H NMR (300 MHz, Methanol-d4) δ 7.75 (dd, J = |
| | hydroxyphenyl)pyridazin- | | 8.4, 1.6 Hz, 1H), 7.48 (d, J = 8.4 Hz, 1H), 7.41 (s, |
| | 4-yl]oxy]ethyl)phenyl] | | 1H), 7.36-7.12 (m, 5H), 7.00-6.83 (m, 3H), |
| | methyl]-3-[2-(2-[[2-(2,6- | | 6.77 (dd, J = 8.4, 2.2 Hz, 1H), 5.06-4.98 (m, 1H), |
| | dioxopiperidin-3-yl)-1,3- | | 4.44 (t, J = 6.5 Hz, 2H), 4.37 (s, 2H), 3.78 (t, J = |
| | dioxoisoindol-5- | | 5.8 Hz, 2H), 3.66-3.56 (d, J = 2.6 Hz, 6H), 3.30- |
| | yl]amino]ethoxy)ethoxy] | | 3.26 (m, 2H), 3.16 (t, J = 6.5 Hz, 2H), 2.91- |
| | propanamide | | 2.58 (m, 3H), 2.50 (t, J = 5.8 Hz, 2H), 2.18-1.91 |
| | | | (m, 1H) |
| 71 | N-[[4-(2-[[3-amino-6-(2- | 748.50 | ¹H NMR (300 MHz, Methanol-d4) d 7.64 (dd, J = |
| | hydroxyphenyl)pyridazin- | | 8.1, 1.6 Hz, 1H), 7.57-7.48 (m, 2H), 7.42-7.30 |
| | 4-yl]oxy]ethyl) | | (m, 4H), 7.30-7.22 (m, 2H), 7.02-6.99 (m, 4H), |
| | phenyl]methyl]-9- | | 5.04 (dd, J = 12.3, 5.4 Hz, 1H), 4.56 (t, J = 6.7 Hz, |
| | [[2-(2,6-dioxopiperidin- | | 2H), 4.34 (s, 2H), 3.30-3.16 (m, 4H), 2.95-2.60 |
| | 3-yl)-1,3-dioxoisoindol-4- | | (m, 3H), 2.23 (t, J = 7.3 Hz, 2H), 2.12 (m,1H), |
| | yl]amino]nonanamide | | 1.71-1.54 (m, 4H), 1.58-1.30 (m, 8H). |
| 72 | N-[[4-(2-[[3-amino-6-(2- | 776.55 | ¹H NMR (300 MHz, Methanol-d4) δ 7.65 (dd, J = |
| | hydroxyphenyl)pyridazin- | | 8.1, 1.7 Hz, 1H), 7.59-7.47 (m, 2H), 7.41-7.31 |
| | 4-yl]oxy]ethyl) | | (m, 3H), 7.30-7.21 (m, 2H), 7.09-6.89 (m, 4H), |
| | phenyl]methyl]-11- | | 5.05 (dd, J = 12.3, 5.5 Hz, 1H), 4.56 (t, J = 6.7 Hz, |
| | 2-(2,6- | | 2H), 4.34 (s, 2H), 3.24 (dd, J = 13.9, 7.3 Hz, 4H), |
| | dioxopiperidin-3-yl)-1,3- | | 2.93-2.44 (m, 3H), 2.22 (t, J = 7.3 Hz, 2H), 2.15- |
| | dioxoisoindol-4- | | 2.00 (m, 1H), 1.64 (q, J = 7.2 Hz, 4H), 1.28-1.50 |
| | yl]amino]undecanamide | | (m,12H) |
| 73 | N-(4-(2-((3-amino-6-(2- | 748.50 | ¹H NMR (300 MHz, methanol-d4) δ 7.78-7.45 |
| | hydroxyphenyl)pyridazin- | | (m, 5H), 7.43-7.15 (m, 5H), 7.08-6.89 (m, 3H), |
| | 4-yl)oxy)ethyl)benzyl)-9- | | 7.09-6.89 (m, 1H), 5.04 (dd, J = 12.3, 5.4 Hz, 1H), |
| | ((2-(2,6-dioxopiperidin-3- | | 4.56 (t, J = 6.7 Hz, 2H), 4.34 (s, 2H), 3.21 (q, J = |
| | yl)-1,3-dioxoisoindolin-5- | | 7.3 Hz, 3H), 2.97-2.59 (m, 4H), 2.24 (t, J = 7.3 |
| | yl)amino)nonanamide | | Hz, 2H), 2.11 (s, 1H), 1.78-1.54 (m, 4H), 1.50- |
| | | | 1.28 (m, 8H) |
| 74 | N-(4-(2-((3-amino-6-(2- | 752.15 | ¹H NMR (400 MHz, DMSO-d6) δ 7.67 (d, J = 7.8 |
| | hydroxyphenyl)pyridazin- | | Hz, 1H), 7.62 (s, 1H), 7.57 (dd, J = 8.6, 7.1 Hz, |
| | 4-yl)oxy)ethyl)benzyl)-3- | | 1H), 7.40-7.27 (m, 3H), 7.19 (d, J = 7.9 Hz, 2H), |
| | (2-(2-((2-(2,6- | | 7.11 (d, J = 8.6 Hz, 1H), 7.06-6.91 (m, 3H), 5.04 |
| | dioxopiperidin-3-yl)-1,3- | | (dd, J = 12.8, 5.4 Hz, 1H), 4.49 (t, J = 6.8 Hz, 2H), |
| | dioxoisoindolin-4- | | 4.23 (s, 2H), 3.61-3.41 (m, 9H), 3.12 (t, J = 6.7 |
| | yl)amino)ethoxy)ethoxy) | | Hz, 2H), 2.95-2.80 (m, 1H), 2.77 (s, 1H), 2.63- |
| | propanamide | | 2.53 (m, 2H), 2.36 (t, J = 6.3 Hz, 2H), 2.01-1.95 |
| | | | (m, 1H) |
| 106 | N-(4-(2-((3-amino-6-(2- | 678.3 | 1H NMR (300 MHz, DMSO-d6) δ 14.35 (s, 1H), |
| | hydroxyphenyl)pyridazin- | | 11.16 (s, 1H), 8.36 (s, 1H), 8.24 (s, 1H), 7.93 (d, J = |
| | 4-yl)oxy)ethyl)benzyl)-4- | | 8.1 Hz, 1H), 7.79 (t, J = 8.1 Hz, 1H), 7.59 (s, |
| | ((3-(2,6-dioxopiperidin-3- | | 1H), 7.34 (d, J = 8.0 Hz, 2H), 7.21 (dq, J = 6.3, |
| | yl)-4-oxo-3,4- | | 3.5, 2.4 Hz, 4H), 6.98 (d, J = 8.6 Hz, 1H), 6.88 (d, |
| | dihydrobenzo[d][1,2,3] | | J = 7.9 Hz, 2H), 6.52 (s, 2H), 5.83 (d, J = 5.8 Hz, |
| | triazin-5-yl)amino) | | 1H), 4.43 (t, J = 6.9 Hz, 2H), 4.24 (d, J = 5.7 Hz, |
| | butanamide | | 2H), 3.25 (t, J = 6.7 Hz, 2H), 3.11 (t, J = 6.7 Hz, |
| | | | 2H), 2.75-2.59 (m, 2H), 2.25 (t, J = 6.9 Hz, 4H), |
| | | | 1.90-1.79 (m, 2H). |
| 111 | N-(4-(2-((3-amino-6-(2- | 664.3 | 1H NMR (300 MHz, DMSO-d6) δ 14.45 (s, 1H), |
| | hydroxyphenyl)pyridazin- | | 11.25 (s, 1H), 8.53 (d, J = 6.0 Hz, 1H), 8.40 (s, |
| | 4-yl)oxy)ethyl)benzyl)-3- | | 1H), 8.01 (d, J = 7.9 Hz, 1H), 7.88 (t, J = 8.1 Hz, |
| | ((3-(2,6-dioxopiperidin-3- | | 1H), 7.66 (s, 1H), 7.38 (d, J = 7.9 Hz, 2H), 7.28 |
| | yl)-4-oxo-3,4- | | (dd, J = 9.9, 7.8 Hz, 4H), 7.10 (d, J = 8.6 Hz, 1H), |
| | dihydrobenzo[d][1,2,3] | | 6.95 (d, J = 7.8 Hz, 2H), 6.57 (s, 2H), 6.01-5.89 |
| | triazin-5-yl)amino) | | (m, 1H), 4.49 (t, J = 6.9 Hz, 2H), 4.33 (d, J = 5.5 |
| | propanamide | | Hz, 2H), 3.59 (d, J = 6.1 Hz, 2H), 3.18 (t, J = 6.9 |
| | | | Hz, 2H), 3.00 (d, J = 14.3 Hz, 1H), 2.82-2.68 (m, |
| | | | 3H), 2.35 (s, 2H). |
| 125 | N-(4-(2-((3-amino-6-(2- | 692.5 | 1H NMR (300 MHz, DMSO-d6) δ 8.32 (m, 2H), |
| | hydroxyphenyl)pyridazin- | | 8.04-7.80 (m, 2H), 7.59 (s, 1H), 7.41-7.14 (m, |
| | 4-yl)oxy)ethyl)benzyl)-5- | | 7H), 7.04 (s, 1H),6.92-6.87 (m, 2H), 6.51 (s, 2H), |
| | ((3-(2,6-dioxopiperidin-3- | | 5.85 (dd, J = 12.1,5.4 Hz, 1H), 4.46-4.42 (m,2H), |
| | yl)-4-oxo-3,4- | | 4.30-4.24 (m, 2H), 3.30-3.20 (m, 2H), 3.20-3.11 |
| | dihydrobenzo[d][1,2,3] | | (m, 2H),3.05-2.96 (m, 2H), 2.77-2.57 (m, 2H), |
| | triazin-6-yl)amino) | | 2.26-2.13 (m, 3H), 1.73-1.55 (m,4H). |
| | pentanamide | | |
| 154 | N-(4-(2-((3-amino-6-(2- | 691.8 | 1H NMR (400 MHz, DMSO-d6) δ 14.38 (s, 1H), |
| | hydroxyphenyl)pyridazin- | | 11.05 (s, 1H), 8.43-8.24 (m, 2H), 7.94 (dd, J = |
| | 4-yl)oxy)ethyl)benzyl)-5- | | 8.0, 1.6 Hz, 1H), 7.61-7.52 (m, 2H), 7.33 (d, J = |
| | ((2-(2,6-dioxopiperidin-3- | | 7.9 Hz, 2H), 7.28-7.17 (m, 3H), 7.13 (t, J = 5.4 |
| | yl)-1,3-dioxoisoindolin-5- | | Hz, 1H), 6.95 (d, J = 2.1 Hz, 1H), 6.91-6.81 (m, |
| | yl)amino)pentanamide | | 3H), 6.50 (s, 2H), 5.03 (dd, J = 12.9, 5.4 Hz, 1H), |
| | | | 4.42 (t, J = 6.9 Hz, 2H), 4.24 (d, J = 5.9 Hz, 2H), |
| | | | 3.21-3.14 (m, 2H), 3.14-3.07 (m, 2H), 2.90- |
| | | | 2.80 (m, 1H), 2.62-2.53 (m, 2H), 2.18 (t, J = 7.1 |
| | | | Hz, 2H), 2.04-1.93 (m, 1H), 1.69-1.52 (m, 4H). |
| 155 | N-(4-(2-((3-amino-6-(2- | 678.2 | 1H NMR (400 MHz, DMSO-d6) δ 14.37 (s, 1H), |
| | hydroxyphenyl)pyridazin- | | 10.97 (s, 1H), 8.30 (t, J = 6.2 Hz, 1H), 7.94 (dd, J = |
| | 4-yl)oxy)ethyl)benzyl)-5- | | 8.0, 1.7 Hz, 1H), 7.59 (s, 1H), 7.40-7.28 (m, |
| | ((2-(2,6-dioxopiperidin-3- | | 3H), 7.26-7.15 (m, 3H), 6.91-6.81 (m, 2H), |
| | yl)-3-oxoisoindolin-4- | | 6.65 (d, J = 7.4 Hz, 1H), 6.59 (dd, J = 7.0, 5.0 Hz, |
| | yl)amino)pentanamide | | 2H), 6.50 (s, 2H), 4.97 (dd, J = 13.2, 5.1 Hz, 1H), |
| | | | 4.42 (t, J = 6.9 Hz, 2H), 4.37-4.11 (m, 4H), 3.20 |
| | | | (q, J = 6.3 Hz, 2H), 3.11 (t, J = 6.8 Hz, 2H), 2.88 |
| | | | (ddd, J = 18.2, 12.7, 5.2 Hz, 1H), 2.70-2.54 (m, |
| | | | 1H), 2.42-2.29 (m, 1H), 2.18 (t, J = 6.9 Hz, 2H), |
| | | | 2.06-1.86 (m, 1H), 1.71-1.48 (m, 4H). |
| 152 | N-(4-(2-((3-amino-6-(2- | 679.1 | 1H NMR (400 MHz, DMSO-d6) δ 14.38 (s, 1H), |
| | hydroxyphenyl)pyridazin- | | 10.97 (s, 1H), 8.42-8.22 (m, 1H), 7.94 (dd, J = |
| | 4-yl)oxy)ethyl)benzyl)-5- | | 8.0, 1.6 Hz, 1H), 7.59 (s, 1H), 7.52 (t, J = 7.9 Hz, |
| | ((2-(2,6-dioxopiperidin-3- | | 1H), 7.33 (d, J = 7.9 Hz, 2H), 7.26-7.17 (m, 3H), |
| | yl)-3-oxoisoindolin-4- | | 7.10 (d, J = 7.5 Hz, 1H), 7.03 (d, J = 8.3 Hz, 1H), |
| | yl)oxy)pentanamide | | 6.91-6.82 (m, 2H), 6.50 (s, 2H), 4.98 (dd, J = |
| | | | 13.2, 5.1 Hz, 1H), 4.52-4.31 (m, 3H), 4.30-4.17 |
| | | | (m, 3H), 4.09 (d, J = 5.8 Hz, 2H), 3.11 (t, J = 6.9 |
| | | | Hz, 2H), 2.87 (ddd, J = 17.3, 13.7, 5.4 Hz, 1H), |
| | | | 2.72-2.54 (m, 1H), 2.43-2.30 (m, 1H), 2.29- |
| | | | 2.17 (m, 2H), 1.96 (dtd, J = 11.7, 6.6, 5.9, 3.3 Hz, |
| | | | 1H), 1.81-1.63 (m, 4H). |
| 143 | N-(4-(2-((3-amino-6-(2- | 719.3 | 1H NMR (400 MHz, DMSO-d6) δ 14.37 (s, 1H), |
| | hydroxyphenyl)pyridazin- | | 11.08 (s, 1H), 8.33 (t, J = 6.2 Hz, 1H), 7.94 (dd, J = |
| | 4-yl)oxy)ethyl)benzyl)-2- | | 8.0, 1.6 Hz, 1H), 7.70 (dd, J = 8.4, 7.2 Hz, 1H), |
| | (4-(2-(2,6-dioxopiperidin- | | 7.59 (s, 1H), 7.39-7.31 (m, 4H), 7.27-7.19 (m, |
| | 3-yl)-1,3-dioxoisoindolin- | | 3H), 6.91-6.82 (m, 2H), 6.50 (s, 2H), 5.09 (dd, J = |
| | 4-yl)piperazin-1- | | 12.9, 5.4 Hz, 1H), 4.43 (t, J = 6.9 Hz, 2H), 4.30 |
| | yl)acetamide | | (d, J = 6.1 Hz, 2H), 3.32 (s, 4H), 3.12 (t, J = 6.8 |
| | | | Hz, 2H), 3.06 (s, 2H), 2.94-2.80 (m, 1H), 2.65 (s, |
| | | | 4H), 2.59 (d, J = 16.5 Hz, 2H), 2.07-1.97 (m, |
| | | | 1H). |
| 164 | (2S,4R)-1-((S)-1-(4-(2-((3- | 908.8 | 1H NMR (300 MHz, DMSO-d6) δ 14.38 (s, 1H), |
| | amino-6-(2- | | 8.97 (s, 1H), 8.59 (s, 1H), 8.27 (t, J = 6.1 Hz, 1H), |
| | hydroxyphenyl)pyridazin- | | 7.94 (d, J = 7.9 Hz, 1H), 7.59 (s, 1H), 7.50 (d, J = |
| | 4-yl)oxy)ethyl)phenyl)-12- | | 9.4 Hz, 1H), 7.34 (d, J = 17.1 Hz, 6H), 7.22 (d, J = |
| | (tert-butyl)-3,10-dioxo-5,8- | | 8.0 Hz, 3H), 6.86 (t, J = 8.0 Hz, 2H), 6.50 (s, 2H), |
| | dioxa-2,11-diazatridecan- | | 5.18 (d, J = 3.5 Hz, 1H), 4.58 (d, J = 9.6 Hz, 1H), |
| | 13-oyl)-4-hydroxy-N-(4-(4- | | 4.40 (dd, J = 19.0, 11.7 Hz, 3H), 4.27 (d, J = 5.9 |
| | methylthiazol-5- | | Hz, 5H), 3.96 (d, J = 5.0 Hz, 4H), 3.64 (s, 6H), |
| | yl)benzyl)pyrrolidine-2- | | 3.11 (s, 2H), 2.43 (s, 3H), 2.05 (d, J = 10.2 Hz, |
| | carboxamide | | 1H), 1.92 (s, 1H), 0.92 (s, 9H). |
| 168 | N1-(4-(2-((3-amino-6-(2- | 1063.4 | 1H NMR (400 MHz, DMSO-d6) δ 14.38 (s, 1H), |
| | hydroxyphenyl)pyridazin- | | 8.97 (s, 1H), 8.59 (t, J = 6.0 Hz, 1H), 8.20 (t, J = |
| | 4-yl)oxy)ethyl)benzyl)- | | 6.2 Hz, 1H), 7.94 (dd, J = 8.0, 1.6 Hz, 1H), 7.59 |
| | N17-((S)-1-((2S,4R)-4- | | (s, 1H), 7.47-7.35 (m, 5H), 7.34 (d, J = 7.9 Hz, |
| | hydroxy-2-((4-(4- | | 2H), 7.27-7.18 (m, 3H), 6.91-6.82 (m, 2H), |
| | methylthiazol-5-yl) | | 6.50 (s, 2H), 5.16 (d, J = 3.5 Hz, 1H), 4.56 (d, J = |
| | benzyl)carbamoyl)pyrrolidin- | | 9.6 Hz, 1H), 4.48-4.34 (m, 5H), 4.30-4.20 (m, |
| | 1-yl)-3,3-dimethyl- | | 3H), 3.94 (d, J = 10.6 Hz, 4H), 3.65-3.58 (m, |
| | 1-oxobutan-2-yl)- | | 2H), 3.56-3.52 (m, 4H), 3.51-3.49 (m, 4H), |
| | 3,6,9,12,15- | | 3.48-3.46 (m, 2H), 3.45-3.34 (m, 6H), 3.11 (t, |
| | pentaoxaheptadecanedia | | J = 6.8 Hz, 2H), 2.44 (s, 3H), 2.05 (t, J = 10.5 Hz, |
| | | | 1H), 1.90 (ddd, J = 13.0, 8.8, 4.4 Hz, 1H), 0.94 (s, |
| | | | 9H). |
| 165 | (2S,4R)-1-((S)-2-(2-(2-((4- | 865.4 | 1H NMR (300 MHz, DMSO-d6) δ 14.45 (s, 1H), |
| | (2-((3-amino-6-(2- | | 9.05 (s, 1H), 8.66 (q, J = 6.2 Hz, 2H), 7.95 (dd, J = |
| | hydroxyphenyl)pyridazin-4- | | 39.7, 8.6 Hz, 2H), 7.58 (d, J = 51.4 Hz, 2H), 7.47 |
| | yl)oxy)ethyl)benzyl)amino)- | | (s, 3H), 7.42 (d, J = 8.0 Hz, 2H), 7.30 (d, J = 7.0 |
| | 2-oxoethoxy)acetamido)- | | Hz, 3H), 6.93 (t, J = 8.0 Hz, 2H), 6.58 (s, 2H), 5.24 |
| | 3,3-dimethylbutanoyl)-4- | | (s, 1H), 4.71-4.22 (m, 9H), 4.13 (d, J = 9.9 Hz, |
| | hydroxy-N-(4-(4- | | 4H), 3.70 (t, J = 10.2 Hz, 2H), 3.18 (t, J = 6.8 Hz, |
| | methylthiazol-5-yl) | | 2H),2.51 (s, 3H), 2.19-1.92 (m, 2H), 1.02 (s, |
| | benzyl)pyrrolidine-2- | | 9H). |
| | carboxamide | | |
| 169 | N1-(4-(2-((3-amino-6-(2- | 989.5 | 1H NMR (400 MHz, DMSO-d6) δ 14.39 (s, 1H), |
| | hydroxyphenyl)pyridazin- | | 8.98 (s, 1H), 8.48 (t, J = 6.0 Hz, 1H), 8.25 (t, J = |
| | 4-yl)oxy)ethyl)benzyl)- | | 6.1 Hz, 1H), 7.98-7.84 (m, 2H), 7.59 (s, 1H), |
| | N15-((S)-1-((2S,4R)-4- | | 7.45-7.29 (m, 6H), 7.28-7.14 (m, 3H), 6.86 (t, J = |
| | hydroxy-2-((4-(4- | | 7.9 Hz, 2H), 6.50 (s, 2H), 5.11 (s, 1H), 4.46- |
| | methylthiazol-5-yl) | | 4.25 (m, 7H), 4.22 (d, J = 5.9 Hz, 2H), 3.65 (d, J = |
| | benzyl)carbamoyl) | | 4.0 Hz, 2H), 3.12 (t, J = 6.8 Hz, 2H), 2.45 (s, 3H), |
| | pyrrolidin-1-yl)-3-methyl- | | 2.10 (t, J = 7.3 Hz, 5H), 1.99-1.61 (m, 2H), 1.49 |
| | 1-oxobutan-2-yl) | | (s, 4H), 1.22 (s, 18H), 0.85 (dd, J = 17.9, 6.7 Hz, |
| | pentadecanediamide | | 6H). |
| 172 | N1-(4-(2-((3-amino-6-(2- | 1045.6 | 1H NMR (300 MHz, DMSO-d6) δ 14.31 (s, 1H), |
| | hydroxyphenyl)pyridazin- | | 9.06 (s, 1H), 8.45 (d, J = 7.8 Hz, 1H), 8.33 (t, J = |
| | 4-yl)oxy)ethyl)benzyl)- | | 6.0 Hz, 1H), 7.99 (d, J = 7.9 Hz, 1H), 7.86 (d, J = |
| | N17-((S)-1-((2S,4R)-4- | | 9.2 Hz, 1H), 7.68 (s, 1H), 7.46 (dt, J = 17.8, 8.4 |
| | hydroxy-2-(((S)-1-(4-(4- | | Hz, 6H), 7.37-7.22 (m, 3H), 6.95 (t, J = 8.1 Hz, |
| | methylthiazoi-5- | | 2H), 6.67 (s, 2H), 5.17 (s, 1H), 4.99 (p, J = 6.8 Hz, |
| | yl)phenyl)ethyl)carbamoyl) | | 1H), 4.64-4.43 (m, 4H), 4.38-4.26 (m, 3H), |
| | pyrrolidin-1-yl)-3,3- | | 3.68 (d, J = 3.5 Hz, 2H), 3.19 (t, J = 6.9 Hz, 2H), |
| | dimethyl-1-oxobutan-2- | | 2.53 (s, 3H), 2.32 (dt, J = 14.4, 7.4 Hz, 1H), 2.24- |
| | yl)heptadecanediamide | | 2.01 (m, 3H), 1.86 (ddd, J = 12.9, 8.5, 4.6 Hz, 1H), |
| | | | 1.63-1.49 (m, 4H), 1.45 (d, J = 7.0 Hz, 3H), 1.30 |
| | | | (S,23H), 1.00 (s, 9H). |
| 173 | N1-(4-(2-((3-amino-6-(2- | 1017.6 | 1H NMR (400 MHz, DMSO-d6) δ 8.98 (s, 1H), |
| | hydroxyphenyl)pyridazin- | | 8.48 (t, J = 6.0 Hz, 1H), 8.26 (t, J = 6.0 Hz, 1H), |
| | 4-yl)oxy)ethyl)benzyl)- | | 7.94 (d, J = 8.9 Hz, 1H), 7.80 (s, 1H), 7.62 (s, 1H), |
| | N17-((S)-1-((2S,4R)-4- | | 7.44-7.26 (m, 7H), 7.19 (d, J = 7.9 Hz, 2H), 6.99- |
| | hydroxy-2-((4-(4- | | 6.86 (m, 3H), 5.11 (s, 1H), 4.47 (t, J = 6.9 Hz, |
| | methylthiazol-5-yl) | | 2H), 4.43-4.25 (m, 5H), 4.22 (d, J = 5.9 Hz, 2H), |
| | benzyl)carbamoyl) | | 3.73-3.54 (m, 2H), 3.13 (d, J = 6.9 Hz, 2H), 2.45 |
| | pyrrolidin-1-yl)-3-methyl- | | (s, 3H), 2.25-2.00 (m, 5H), 1.97-1.83 (m, 2H), |
| | 1-oxobutan-2-yl) | | 1.49 (t, J = 8.1 Hz, 4H), 1.22 (d, J = 2.7 Hz, 22H), |
| | heptadecanediamide | | 0.85 (dd, J = 18.0, 6.6 Hz, 6H). |

### Example 5. Preparation of N-[[4-(2-[[3-amino-6-(2-hydroxyphenyl)pyridazin-4-yl]oxy]ethyl)phenyl]methyl]-5-[[2-(2,6-dioxopiperidin-3-yl)-1,3-dioxoisoindol-4-yl]amino]pentanamide (compound 4)

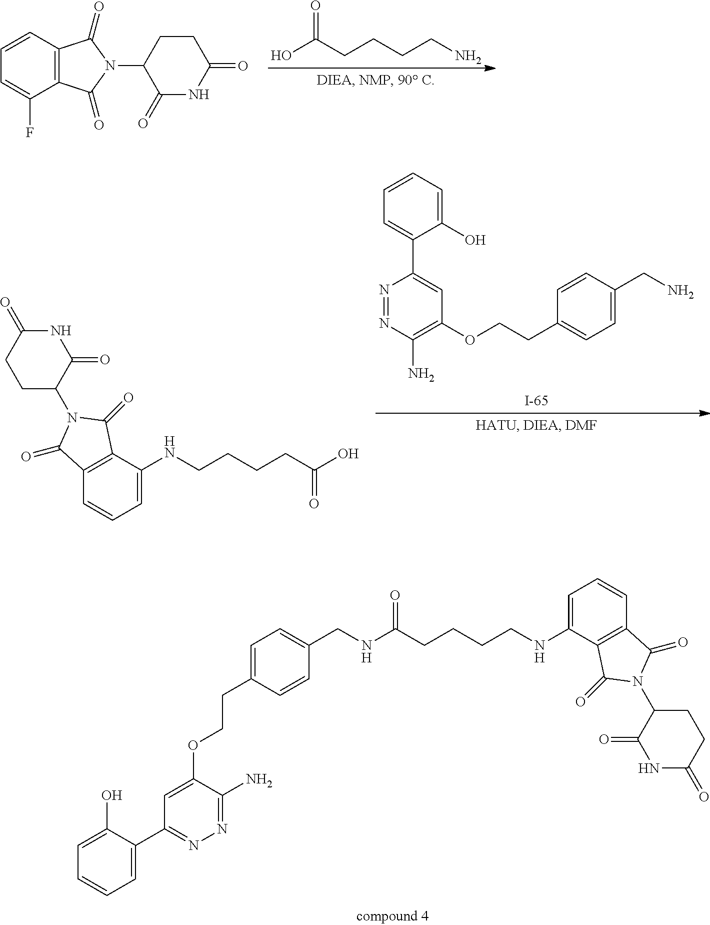

### Step 1: Preparation of 5-((2-(2,6-dioxopiperidin-3-yl)-1,3-dioxoisoindolin-4-yl)amino)pentanoic acid

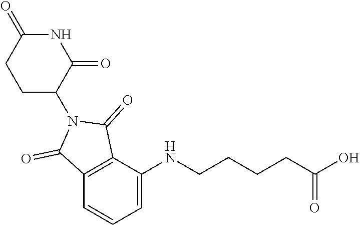 [SMILES: O=C(O)CCCCNc1cccc2c1C(=O)N(C1CCC(=O)NC1=O)C2=O]

To a stirred solution of 2-(2,6-dioxopiperidin-3-yl)-4-fluoroisoindole-1,3-dione (300.00 mg, 1.086 mmol, 1 equiv) and 5-aminovaleric acid (152.68 mg, 1.303 mmol, 1.20 equiv) in NMP (1.00 mL) was added DIEA (701.84 mg, 5.430 mmol, 5.00 equiv). The resulting mixture was stirred for 2 h at 90° C. under an atmosphere of dry nitrogen. The mixture was allowed to cool to room temperature and then purified by reversed phase flash chromatography to afford 5-((2-(2,6-dioxopiperidin-3-yl)-1,3-dioxoisoindolin-4-yl)amino)pentanoic acid (87 mg, 20.60%) as a white solid. LCMS (ESI) m/z: [M+H]⁺=373.

### Step 2: Preparation of N-[[4-(2-[[3-amino-6-(2-hydroxyphenyl)pyridazin-4-yl]oxy]ethyl)phenyl]methyl]-5-[[2-(2,6-dioxopiperidin-3-yl)-1,3-dioxoisoindol-4-yl]amino]pentanamide (compound 4)

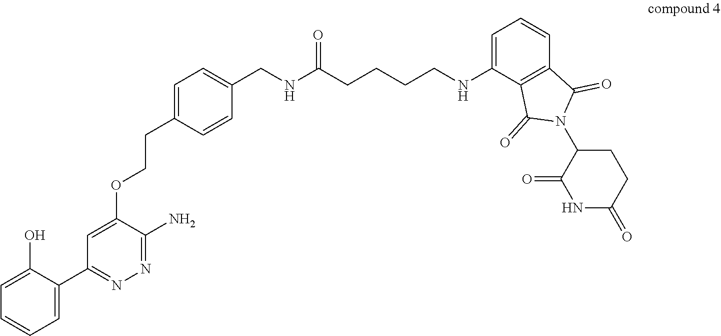 [SMILES: Nc1nnc(-c2ccccc2O)cc1OCCc1ccc(CNC(=O)CCCCNc2cccc3c2C(=O)N(C2CCC(=O)NC2=O)C3=O)cc1]

To a stirred solution of 5-((2-(2,6-dioxopiperidin-3-yl)-1,3-dioxoisoindolin-4-yl)amino)pentanoic acid (50.00 mg, 0.134 mmol, 1.00 equiv) and 2-(6-amino-5-[2-[4-(aminomethyl)phenyl]ethoxy]pyridazin-3-yl)phenol (I-65, 54.06 mg, 0.161 mmol, 1.20 equiv) in DMF (2.00 mL) were added DIEA (86.54 mg, 0.670 mmol, 5.00 equiv) and HATU (101.84 mg, 0.268 mmol, 2.00 equiv). The mixture was stirred for 2 h at room temperature under an atmosphere of dry nitrogen. The resulting mixture was diluted with water (10 mL) and extracted with EtOAc (3×10 mL). The combined organic layers were washed with brine (20 mL), and dried over anhydrous Na₂SO₄. After filtration, the filtrate was concentrated under reduced pressure to afford the crude product which was purified by reversed-phase preparative HPLC to afford compound 4 (13.2 mg, 14.2%) as a white solid. ¹H NMR (400 MHz, DMSO-d6) δ 14.29 (s, 1H), 11.11 (s, 1H), 8.32 (t, J=6.0 Hz, 1H), 7.94 (dd, J=8.0, 1.6 Hz, 1H), 7.62-7.48 (m, 2H), 7.34 (d, J=8.0 Hz, 2H), 7.29-7.16 (m, 3H), 7.09 (d, J=8.6 Hz, 1H), 7.02 (d, J=7.0 Hz, 1H), 6.87 (td, J=8.0, 7.3, 1.6 Hz, 2H), 6.63-6.48 (m, 3H), 5.05 (dd, J=12.9, 5.4 Hz, 1H), 4.43 (t, J=6.8 Hz, 2H), 4.24 (d, J=5.9 Hz, 2H), 3.31 (d, J=6.1 Hz, 2H), 3.11 (t, J=6.8 Hz, 2H), 2.94-2.80 (m, 1H), 2.63-2.53 (m, 2H), 2.18 (t, J=6.8 Hz, 2H), 2.02 (ddq, J=10.0, 5.5, 2.7 Hz, 1H), 1.67-1.48 (m, 4H). LCMS (ESI) m/z: [M+H]⁺=692.30.

### Example 6. Preparation of N-(4-(2-((3-amino-6-(2-hydroxyphenyl)pyridazin-4-yl)oxy)ethyl)benzyl)-5-((2-(2,6-dioxopiperidin-3-yl)-1,3-dioxoisoindolin-5-yl)oxy)pentanamide (compound 5)

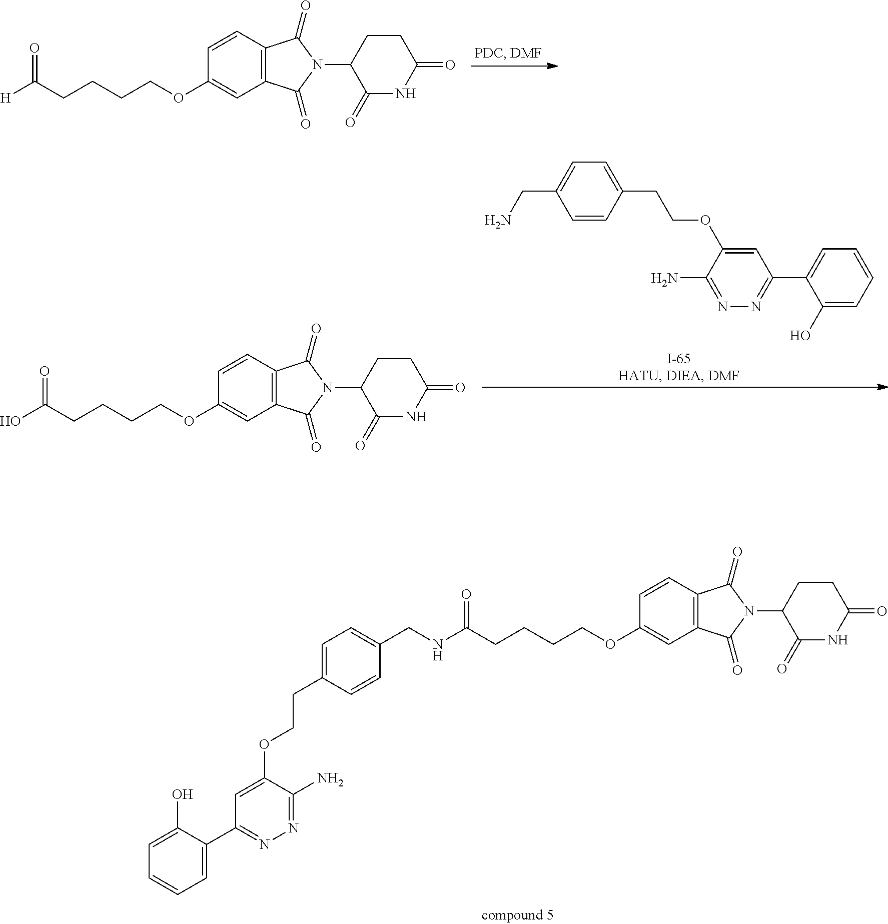 [SMILES: O=CCCCCOc1ccc2c(c1)C(=O)N(C1CCC(=O)NC1=O)C2=O ; O=C(O)CCCCOc1ccc2c(c1)C(=O)N(C1CCC(=O)NC1=O)C2=O ; NCc1ccc(CCOc2cc(-c3ccccc3O)nnc2N)cc1 ; "I-65HATU, DIEA, DMF" ; Nc1nnc(-c2ccccc2O)cc1OCCc1ccc(CNC(=O)CCCCOc2ccc3c(c2)C(=O)N(C2CCC(=O)NC2=O)C3=O)cc1]

### Step 1: Preparation of 5-((2-(2,6-dioxopiperidin-3-yl)-1,3-dioxoisoindolin-5-yl)oxy)pentanoic acid

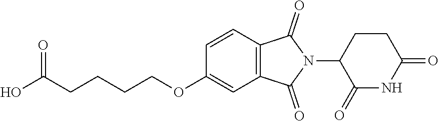 [SMILES: O=C(O)CCCCOc1ccc2c(c1)C(=O)N(C1CCC(=O)NC1=O)C2=O]

To a solution of 5-[[2-(2,6-dioxopiperidin-3-yl)-1,3-dioxoisoindol-5-yl]oxy]pentanal, 40.00 mg, 0.112 mmol, 1.00 equiv) in DMF (1.00 mL) was added PDC (83.99 mg, 0.223 mmol, 2.00 equiv) in portions at room temperature. The resulting mixture was stirred overnight at room temperature and then concentrated. The residue was purified by reversed phase flash chromatography to give 5-((2-(2,6-dioxopiperidin-3-yl)-1,3-dioxoisoindolin-5-yl)oxy)pentanoic acid (30 mg, 66.55%) as a light brown solid. LCMS (ESI) m/z: [M+H]⁺=375.

### Step 2: Preparation of N-(4-(2-((3-amino-6-(2-hydroxyphenyl)pyridazin-4-yl)oxy)ethyl)benzyl)-5-((2-(2,6-dioxopiperidin-3-yl)-1,3-dioxoisoindolin-5-yl)oxy)pentanamide (compound 5)

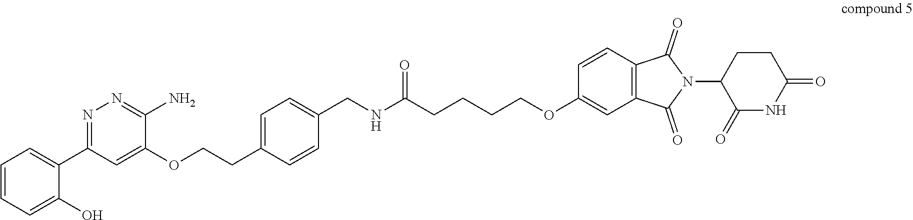 [SMILES: Nc1nnc(-c2ccccc2O)cc1OCCc1ccc(CNC(=O)CCCCOc2ccc3c(c2)C(=O)N(C2CCC(=O)NC2=O)C3=O)cc1]

To a stirred mixture of 5-((2-(2,6-dioxopiperidin-3-yl)-1,3-dioxoisoindolin-5-yl)oxy)pentanoic acid (30.00 mg, 0.080 mmol, 1.00 equiv) and 2-(6-amino-5-[2-[4-(aminomethyl)phenyl]ethoxy]pyridazin-3-yl)phenol (I-65, 29.65 mg, 0.088 mmol, 1.10 equiv) in DMF (1.00 mL) were added HATU (45.71 mg, 0.120 mmol, 1.50 equiv) and DIEA (41.43 mg, 0.321 mmol, 4.00 equiv) dropwise at room temperature. The resulting mixture was stirred for 1 h at room temperature. The resulting mixture was purified by reversed-phase preparative HPLC to afford compound 5 (6.6 mg, 11.69%) as an off-white solid. ¹H NMR (400 MHz, DMSO-d6) δ 11.12 (s, 1H), 8.33 (t, J=6.0 Hz, 1H), 7.94 (d, J=8.0 Hz, 1H), 7.83 (d, J=8.2 Hz, 2H), 7.59 (s, 1H), 7.42 (d, J=2.3 Hz, 1H), 7.34 (dd, J=8.3, 2.5 Hz, 3H), 7.22 (t, J=7.8 Hz, 3H), 6.87 (t, J=8.3 Hz, 2H), 6.51 (s, 2H), 5.12 (dd, J=12.9, 5.4 Hz, 1H), 4.43 (t, J=6.9 Hz, 2H), 4.24 (d, J=5.9 Hz, 2H), 4.18 (t, J=6.1 Hz, 2H), 3.12 (t, J=6.8 Hz, 2H), 2.87 (d, J=12.3 Hz, 1H), 2.60 (d, J=17.8 Hz, 2H), 2.22 (t, J=7.0 Hz, 2H), 2.12-1.97 (m, 1H), 1.72-1.63 (m, 4H). LCMS (ESI) m/z: [M+H]⁺=693.20.

### Example 7. Preparation of N-(4-(2-((3-amino-6-(2-hydroxyphenyl)pyridazin-4-yl) oxy)ethyl)benzyl)-5-((3-(2,6-dioxopiperidin-3-yl)-4-oxo-3,4-dihydrobenzo[d][1,2,3]triazin-5-yl)amino)pentanamide (compound 1)

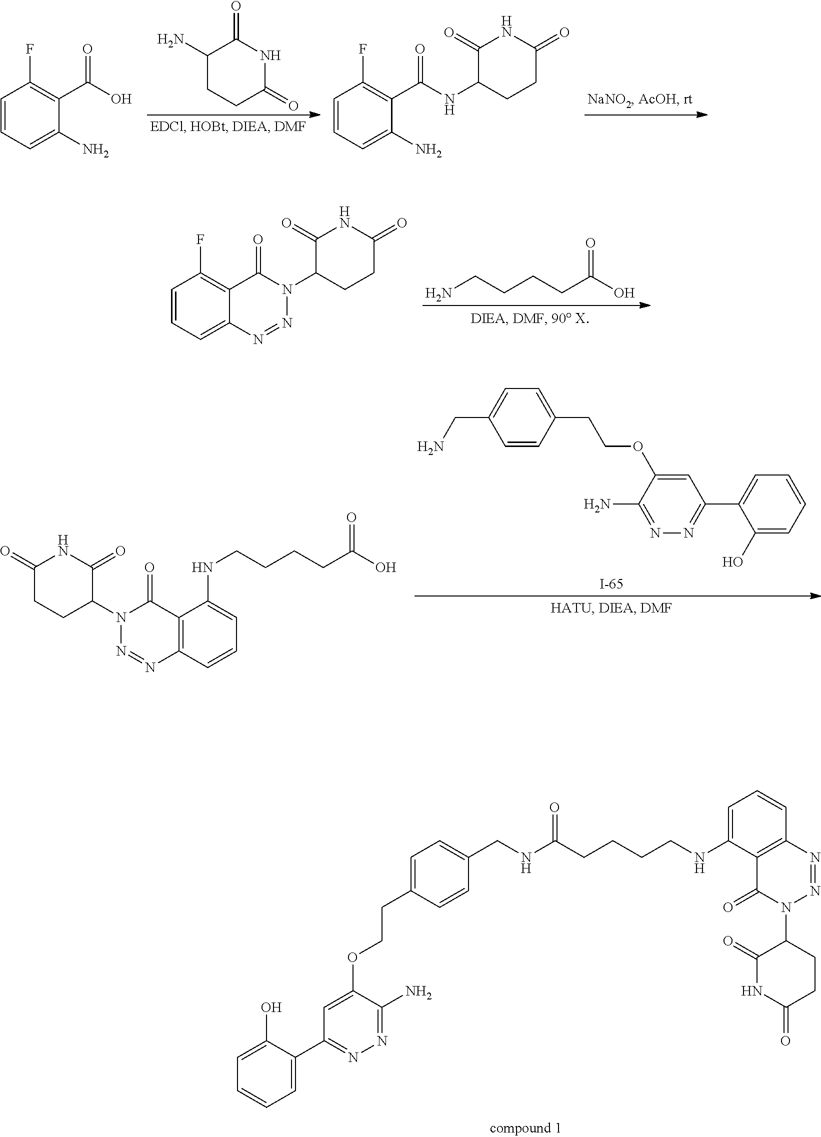

### Step 1: Preparation of 2-amino-N-(2,6-dioxopiperidin-3-yl)-6-fluorobenzamide

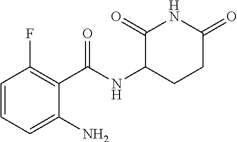 [SMILES: Nc1cccc(F)c1C(=O)NC1CCC(=O)NC1=O]

To a stirred mixture of 2-amino-6-fluorobenzoic acid (1.00 g, 6.446 mmol, 1.00 equiv), 3-aminopiperidine-2,6-dione (908.57 mg, 7.091 mmol, 1.10 equiv), HOBt (958.15 mg, 7.091 mmol, 1.1 equiv) and EDC HCl (1.36 g, 7.091 mmol, 1.1 equiv) in DMF (20 mL) was added DIEA (2.50 g, 19.339 mmol, 3.00 equiv) dropwise at room temperature under an atmosphere of dry nitrogen. The resulting mixture was stirred for 3 h then diluted with EtOAc (100 mL) and washed with brine (2×100 mL). The organic layer was dried over Na₂SO₄ and filtered. The filtrate was concentrated, and the residue purified by reversed phase flash chromatography to afford 2-amino-N-(2,6-dioxopiperidin-3-yl)-6-fluorobenzamide (1.2 g, 63.16%) as an off-white solid. LCMS (ESI) m/z [M+H]⁺=266.

### Step 2: Preparation of 3-(5-fluoro-4-oxobenzo[d][1,2,3]triazin-3(4H)-yl)piperidine-2,6-dione

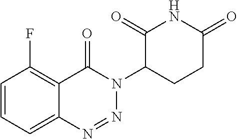 [SMILES: O=C1CCC(n2nnc3cccc(F)c3c2=O)C(=O)N1]

To a solution of 2-amino-N-(2,6-dioxopiperidin-3-yl)-6-fluorobenzamide (1.20 g, 4.524 mmol, 1.00 equiv) in AcOH (10 mL) was added NaNO₂ (530.65 mg, 7.691 mmol, 1.70 equiv) in portions at room temperature under an atmosphere of dry nitrogen. The resulting mixture was stirred for 3 h then filtered to afford 3-(5-fluoro-4-oxobenzo[d][1,2,3]triazin-3(4H)-yl)piperidine-2,6-dione (800 mg, 63.38%) as a white solid. LCMS (ESI) m/z [M+H]⁺=277.

### Step 3: Preparation of 5-((3-(2,6-dioxopiperidin-3-yl)-4-oxo-3,4-dihydrobenzo[d][1,2,3]triazin-5-yl)amino)pentanoic acid

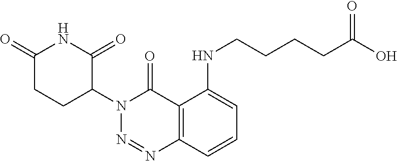 [SMILES: O=C(O)CCCCNc1cccc2nnn(C3CCC(=O)NC3=O)c(=O)c12]

To a stirred mixture of 3-(5-fluoro-4-oxobenzo[d][1,2,3]triazin-3(4H)-yl)piperidine-2,6-dione (200.00 mg, 0.724 mmol, 1.00 equiv) and 5-aminovaleric acid (127.23 mg, 1.086 mmol, 1.50 equiv) in DMF (5 mL) was added DIEA (280.73 mg, 2.172 mmol, 3.00 equiv) dropwise at room temperature under an atmosphere of dry nitrogen. The resulting mixture was stirred for 4 h at 90° C. then diluted with EtOAc (20 mL) and washed with brine (2×20 mL). The organic layer was dried over Na₂SO₄ and filtered. The filtrate was concentrated and the residue purified by reversed phase flash chromatography to afford 5-((3-(2,6-dioxopiperidin-3-yl)-4-oxo-3,4-dihydrobenzo[d][1,2,3]triazin-5-yl)amino)pentanoic acid (75 mg, 27.47%) as a yellow solid. LCMS (ESI) m/z [M+H]⁺=374.

### Step 4: Preparation of N-(4-(2-((3-amino-6-(2-hydroxyphenyl) pyridazin-4-yl)oxy)ethyl) benzyl)-5-((3-(2,6-dioxopiperidin-3-yl)-4-oxo-3,4-dihydrobenzo[d][1,2,3]triazin-5-yl)amino)pentanamide (compound 1)

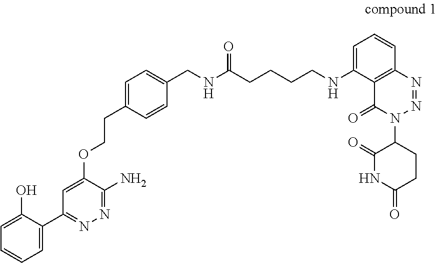 [SMILES: Nc1nnc(-c2ccccc2O)cc1OCCc1ccc(CNC(=O)CCCCNc2cccc3nnn(C4CCC(=O)NC4=O)c(=O)c23)cc1]

To a stirred mixture of 5-((3-(2,6-dioxopiperidin-3-yl)-4-oxo-3,4-dihydrobenzo[d][1,2,3]triazin-5-yl)amino)pentanoic acid (55.00 mg, 0.147 mmol, 1.00 equiv) and 2-(6-amino-5-[2-[4-(aminomethyl)phenyl]ethoxy]pyridazin-3-yl)phenol (I-65, 49.55 mg, 0.147 mmol, 1.00 equiv) in DMF (1.5 mL) were added HATU (67.21 mg, 0.177 mmol, 1.20 equiv) and DIEA (57.12 mg, 0.442 mmol, 3.00 equiv) at room temperature under an atmosphere of dry nitrogen. The resulting mixture was stirred for 2 h then purified by reversed-phase preparative HPLC to afford compound 1 (11.4 mg, 11.2%) as a yellow solid. ¹H NMR (300 MHz, DMSO-d₆) δ 14.32 (brs, 1H), 11.18 (s, 1H), 8.41-8.09 (m, 2H), 7.97-7.72 (m, 2H), 7.60 (s, 1H), 7.34 (d, J=7.9 Hz, 2H), 7.30-7.10 (m, 4H), 7.05-6.77 (m, 3H), 6.63 (s, 1H), 5.87 (dd, J=12.2, 5.3 Hz, 1H), 4.44 (t, J=6.8 Hz, 2H), 4.24 (d, J=5.9 Hz, 2H), 3.25 (d, J=5.6 Hz, 3H), 3.12 (t, J=6.9 Hz, 2H), 2.91 (d, J=13.0 Hz, 1H), 2.75-2.60 (m, 2H), 2.33-2.11 (m, 3H), 1.77-1.47 (m, 4H). LCMS (ESI) m/z [M+H]⁺=692.30.

The following compounds in Table D2 were prepared using procedures similar to those used for the preparation of compound 1.

TABLE D2

| | | LCMS | |
|---|---|---|---|
| No. | Name | (ESI) m/z | ¹H NMR |
| 141 | N-(4-(2-((3-amino-6-(2- | 691.2 | ¹H NMR (300 MHz, DMSO-d6) δ 11.19 (s, 1H), |
| | hydroxyphenyl)pyridazin- | | 8.43-8.15 (m, 1H), 8.33-8.24 (m, 1H), 7.96- |
| | 4-yl)amino)ethyl)benzyl)- | | 7.69 (m, 1H), 7.33-7.13 (m, 6H), 7.05-6.90 (m, |
| | 5-((3-(2,6-dioxopiperidin- | | 2H), 6.88-6.74 (m, 2H), 6.39-6.18 (m, 3H), |
| | 3-yl)-4-oxo-3,4- | | 5.88 (dd, J = 12.1,5.4 Hz, 1H), 4.22 (d, J = 5.9 |
| | dihydrobenzo[d][1,2,3] | | Hz, 2H), 3.55-3.46 (m, 2H), 3.28-3.22 (m, 3H), |
| | triazin-5-yl)amino) | | 2.99-2.86 (m, 3H), 2.78-2.57 (m, 2H), 2.32- |
| | pentanamide | | 2.12 (m, 3H), 1.61 (s, 4H). |
| 142 | N-(4-(2-((3-amino-6-(2- | 705.1 | ¹H NMR (300 MHz, DMSO-d6) δ14.09 (brs, 1H), |
| | hydroxyphenyl)pyridazin- | | 11.24 (s, 1H), 8.51-8.08 (m, 2H), 7.95 (d, J = 7.9 |
| | 4-yl)(methyl)amino)ethyl) | | Hz, 1H), 7.86 (t, J = 8.2 Hz, 1H), 7.50 (s, 1H), 7.37- |
| | benzyl)-5-((3-(2,6- | | 7.09 (m, 6H), 7.08-6.81 (m, 3H), 6.20 (s, 2H), |
| | dioxopiperidin-3-yl)-4- | | 5.94 (dd, J = 12.3, 5.3 Hz, 1H), 4.26 (d, J = 5.8 |
| | oxo-3,4-dihydrobenzo[d] | | Hz, 2H), 3.46 (s, 1H), 3.32 (d, J = 5.5 Hz, 3H), |
| | [1,2,3]triazin-5-yl) | | 3.01 (s, 4H), 2.85 (t, J = 7.7 Hz, 2H), 2.73 (d, J = |
| | amino)pentanamide | | 13.3 Hz, 2H), 2.30-2.12 (m, 3H), 1.69 (s, 4H). |

### Example 8. Preparation of N-(4-(2-((3-amino-6-(2-hydroxyphenyl)pyridazin-4-yl)oxy)ethyl)benzyl)-5-((2-(2,6-dioxopiperidin-3-yl)-1-oxoisoindolin-4-yl)amino)pentanamide (compound 2)

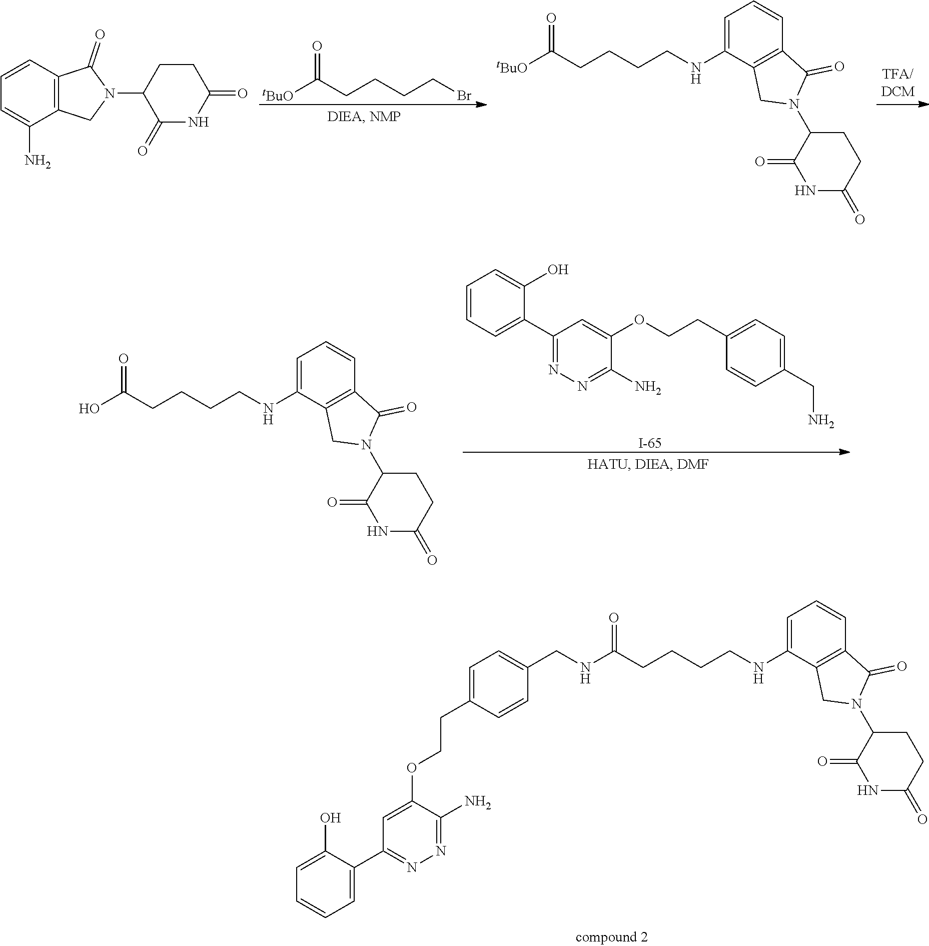

### Step 1: Preparation of tert-butyl 5-((2-(2,6-dioxopiperidin-3-yl)-1-oxoisoindolin-4-yl)amino)pentanoate

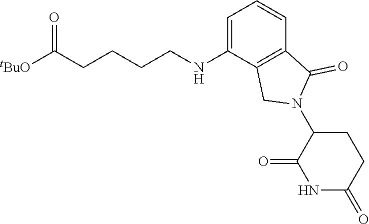 [SMILES: CC(C)(C)OC(=O)CCCCNc1cccc2c1CN(C1CCC(=O)NC1=O)C2=O]

To a stirred mixture of lenalidomide (100.00 mg, 0.386 mmol, 1.00 equiv) and tert-butyl 5-bromopentanoate (109.76 mg, 0.463 mmol, 1.20 equiv) in NMP (1.5 mL) was added DIEA (149.55 mg, 1.157 mmol, 3.00 equiv) dropwise at room temperature under an atmosphere of dry nitrogen. The resulting mixture was stirred overnight at 90° C. The mixture was diluted with EtOAc (10 mL) and it was washed with brine (2×10 mL). The organic layer was dried over Na₂SO₄ and filtered. The filtrate was concentrated and the residue purified by reversed phase flash chromatography to afford tert-butyl 5-((2-(2,6-dioxopiperidin-3-yl)-1-oxoisoindolin-4-yl)amino)pentanoate (93 mg, 53.39%) as a yellow solid. LCMS (ESI) m/z [M+H]⁺=416.

### Step 2: Preparation of 5-((2-(2,6-dioxopiperidin-3-yl)-1-oxoisoindolin-4-yl)amino)pentanoic acid

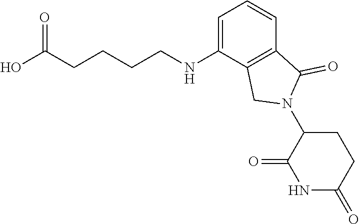 [SMILES: O=C(O)CCCCNc1cccc2c1CN(C1CCC(=O)NC1=O)C2=O]

To a solution of tert-butyl 5-((2-(2,6-dioxopiperidin-3-yl)-1-oxoisoindolin-4-yl)amino)pentanoate (93.00 mg, 0.22 mmol) in DCM was added TFA (0.50 mL) dropwise at room temperature. The resulting mixture was stirred for 1 then concentrated under reduced pressure. The residue was purified by reversed phase flash chromatography to afford 5-((2-(2,6-dioxopiperidin-3-yl)-1-oxoisoindolin-4-yl)amino)pentanoic acid (60 mg, 77.27%) as a yellow solid. LCMS (ESI) m/z [M+H]⁺=360.

### Step 3: Preparation of N-(4-(2-((3-amino-6-(2-hydroxyphenyl)pyridazin-4-yl)oxy)ethyl)benzyl)-5-((2-(2,6-dioxopiperidin-3-yl)-1-oxoisoindolin-4-yl)amino)pentanamide (compound 2)

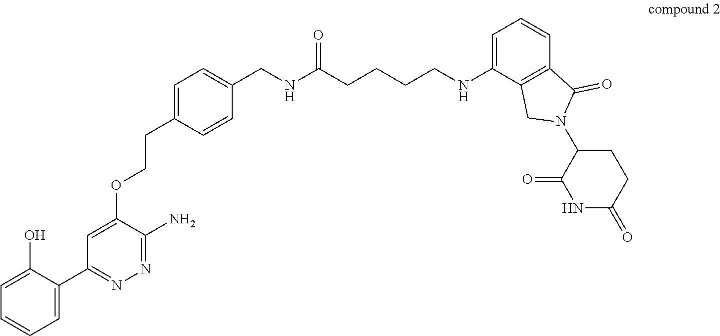 [SMILES: Nc1nnc(-c2ccccc2O)cc1OCCc1ccc(CNC(=O)CCCCNc2cccc3c2CN(C2CCC(=O)NC2=O)C3=O)cc1]

To a stirred mixture of 5-((2-(2,6-dioxopiperidin-3-yl)-1-oxoisoindolin-4-yl)amino)pentanoic acid (25.00 mg, 0.070 mmol, 1.00 equiv) and 2-(6-amino-5-[2-[4-(aminomethyl)phenyl]ethoxy]pyridazin-3-yl)phenol (I-65, 23.40 mg, 0.070 mmol, 1.00 equiv) in DMF (1.0 mL) were added HATU (31.74 mg, 0.083 mmol, 1.20 equiv) and DIEA (44.95 mg, 0.348 mmol, 5.00 equiv) in portions at room temperature. The resulting mixture was stirred for 1 h then purified by reversed-phase preparative HPLC to afford compound 2 (15.2 mg, 31.72%) as a white solid. ¹H NMR (300 MHz, DMSO-d₆) δ 11.02 (s, 1H), 8.34 (t, J=6.0 Hz, 1H), 7.72 (d, J=7.7 Hz, 1H), 7.61 (s, 1H), 7.37-7.25 (m, 4H), 7.18 (d, J=7.9 Hz, 3H), 6.95 (dd, J=11.8, 7.6 Hz, 3H), 6.74 (d, J=8.0 Hz, 1H), 5.10 (dd, J=13.2, 5.1 Hz, 1H), 4.48 (t, J=6.8 Hz, 2H), 4.26-4.14 (m, 4H), 3.12 (t, J=6.6 Hz, 4H), 2.96-2.87 (m, 2H), 2.67-2.56 (m, 1H), 2.40-2.26 (m, 1H), 2.18 (t, J=6.8 Hz, 2H), 2.08-1.97 (m, 1H), 1.72-1.53 (m, 4H). LCMS (ESI) m/z [M+H]⁺=678.20.

The following compounds in Table D3 were prepared using procedures similar to those used for the preparation of compound 2.

TABLE D3

| | | LCMS | |
|---|---|---|---|
| No. | Name | (ESI) m/z | ¹H NMR |
| 135 | N-[[4-(2-[[3-amino-6-(2- | 678.0 | ¹H NMR (300 MHz, DMSO-d6) δ 14.39 (s, 1H), |
| | hydroxyphenyl)pyridazin- | | 10.96 (s, 1H), 8.30 (t, J = 5.9 Hz, 1H), 7.95 (d, J = |
| | 4-yl]oxy]ethyl)phenyl] | | 7.9 Hz, 1H), 7.60 (s, 1H), 7.34 (d, J = 8.0 Hz, 2H), |
| | methyl]-5-[[2-(2,6- | | 7.29-7.12 (m, 4H), 6.97-6.81 (m, 3H), 6.78 (s, |
| | dioxopiperidin-3-yl)-3- | | 1H), 6.52 (d, 2H), 5.90 (t, 1H), 5.07 (dd, J = 13.1, |
| | oxo-1H-isoindol-5-yl] | | 5.0 Hz, 1H), 4.43 (t, J = 6.8 Hz, 2H), 4.33-4.10 |
| | amino]pentanamide | | (m, 4H), 3.17-3.00 (m, 4H), 2.99-2.80 (m, 1H), |
| | | | 2.61 (s, 1H), 2.38-2.32 (m, 1H), 2.18 (t, J = 7.0 |
| | | | Hz, 2H), 2.05-1.88 (m, 1H), 1.75-1.48 (m, 4H). |

### Example 9. Preparation of 4-[[5-([[4-(2-[[3-amino-6-(2-hydroxyphenyl)pyridazin-4-yl]oxy]ethyl)phenyl]methyl]amino)pentyl]oxy]-2-(2,6-dioxopiperidin-3-yl)isoindole-1,3-dione (compound 9)

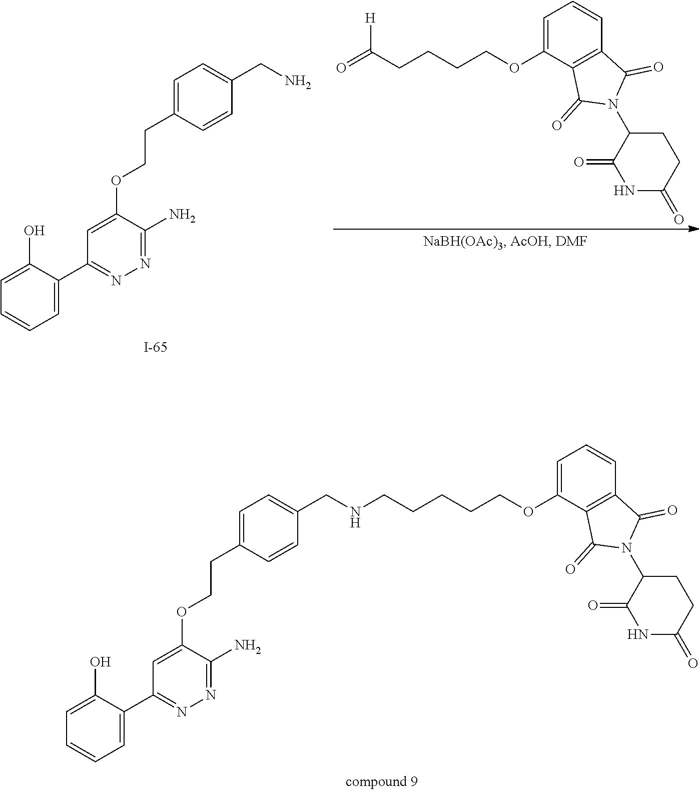

To a stirred solution of 2-(6-amino-5-[2-[4-(aminomethyl)phenyl]ethoxy]pyridazin-3-yl)phenol (I-65, 50.00 mg, 0.149 mmol, 1.00 equiv) and 5-[[2-(2,6-dioxopiperidin-3-yl)-1,3-dioxoisoindol-4-yl]oxy]pentanal (63.92 mg, 0.178 mmol, 1.20 equiv) in DMF (2.00 mL) were added NaBH(OAc)₃ (63.00 mg, 0.372 mmol, 2.50 equiv) and AcOH (one drop). The resulting mixture was stirred overnight at room temperature, then diluted with water (10 mL). The resulting mixture was extracted with EtOAc (3×10 mL). The combined organic layers were washed with brine (2×10 mL), and dried over anhydrous Na₂SO₄. After filtration, the filtrate was concentrated under reduced pressure to afford crude product. The residue was purified by reversed-phase preparative HPLC to afford compound 9 (6.2 mg, 6.13%) as a white solid. ¹H NMR (400 MHz, DMSO-d6) δ 14.36 (s, 1H), 11.12 (s, 1H), 7.96-7.92 (m, 1H), 7.82 (dd, J=8.5, 7.3 Hz, 1H), 7.60 (s, 1H), 7.52 (d, J=8.6 Hz, 1H), 7.48-7.40 (m, 3H), 7.37 (d, J=7.9 Hz, 2H), 7.24 (td, J=7.7, 1.6 Hz, 1H), 6.91-6.84 (m, 2H), 6.52 (s, 2H), 5.08 (dd, J=12.7, 5.4 Hz, 1H), 4.47 (t, J=6.7 Hz, 2H), 4.20 (t, J=6.2 Hz, 2H), 3.94 (s, 2H), 3.15 (t, J=6.6 Hz, 2H), 2.97-2.82 (m, 2H), 2.76 (t, J=7.4 Hz, 2H), 2.61 (dd, J=4.5, 2.5 Hz, 1H), 2.06-1.98 (m, 1H), 1.77 (p, J=6.8 Hz, 2H), 1.61 (q, J=7.6 Hz, 2H), 1.51 (q, J=7.1 Hz, 2H). LCMS (ESI) m/z: [M+H]⁺=679.20.

The following compounds in Table D4 were prepared using procedures similar to those used for the preparation of compound 9.

TABLE D4

| | | LCMS | |
|---|---|---|---|
| | | (ESI) | |
| No. | Name | m/z | ¹H NMR |
| 114 | 4-(3-(3-((4-(2-((3-amino-6-(2- | 690.4 | ¹H NMR (400 MHz, DMSO-d6) δ 14.38 (brs, |
| | hydroxyphenyl)pyridazin-4- | | 1H), 11.06 (s, 1H), 7.93 (dd, J = 8.0, 1.6 Hz, |
| | yl)oxy)ethyl)benzyl)amino) | | 1H), 7.58-7.54 (m, 2H), 7.36 (d, J = 7.6 Hz, |
| | propyl)azetidin-1-yl)-2- | | 2H), 7.31 (d, J = 7.8 Hz, 2H), 7.23 (t, J = 7.1, |
| | (2,6-dioxopiperidin-3-yl) | | Hz, 1H), 7.09 (m, 1H), 6.91-6.80 (m, 2H), |
| | isoindoline-1,3-dione | | 6.75-6.78 (m, 1H), 6.52-6.43 (m, 3H), 5.04 |
| | | | (m, 1H), 4.45 (t, J = 6.9 Hz, 2H), 4.28 (t, J = 8.3 |
| | | | Hz, 2H), 4.12 (s, 2H), 3.86-3.76 (m, 2H), 3.13 |
| | | | (t, J = 6.8 Hz, 2H), 2.90-2.83 (m, 3H), 2.68- |
| | | | 2.58 (m, 3H), 2.46-2.42 (m, 1H), 2.03-1.96 |
| | | | (m, 1H), 1.68-1.55 (m, 4H). |
| 110 | 4-[4-[2-([[4-(2-[[3-amino-6-(2- | 705.3 | ¹H NMR (300 MHz, DMSO-d6) δ 8.24 (s, 2H), |
| | hydroxyphenyl)pyridazin-4- | | 7.93 (dd, J = 8.4, 1.5 Hz, 1H), 7.70 (dd, J = 8.1, |
| | yl]oxy]ethyl)phenyl]methyl] | | 1.6 Hz, 1H), 7.58 (s, 1H), 7.48-7.28 (m, 6H), |
| | amino)ethyl]piperazin- | | 7.24 (td, J = 8.5, 1.6 Hz, 1H), 6.94-6.82 (m, |
| | 1-yl]-2-(2,6-dioxopiperidin- | | 2H), 5.08 (dd, J = 8.4, 1.5 Hz, 1H), 4.46 (t, J = |
| | 3-yl)isoindole-1,3-dione | | 6.6 Hz, 2H), 3.85 (s, 2H), 3.29 (s, 4H), 3.15 (t, |
| | | | J = 6.6 Hz, 2H), 2.81 (d, J = 6.6 Hz, 3H), 2.62 |
| | | | (s, 7H), 2.10-1.94 (m, 1H). |
| 123 | 5-[3-[3-([[4-(2-[[3-amino-6-(2- | 690.3 | ¹H NMR (300 MHz, DMSO-d6) δ 14.25 (brs, |
| | hydroxyphenyl)pyridazin-4- | | 1H), 11.06 (s, 1H), 8.32 (s, 1H, FA), 8.31 (s, |
| | yl]oxy]ethyl)phenyl]methyl] | | 1H), 7.92 (d, J = 8.1 Hz, 1H), 7.66-7.55 (m, |
| | amino)propyl]azetidin- | | 2H), 7.40-7.28 (m, 4H), 7.23 (t, J = 7.7 Hz, |
| | 1-yl]-2-(2,6-dioxopiperidin- | | 1H), 6.95-6.81 (m, 2H), 6.74 (d, J = 2.1 Hz, |
| | 3-yl)isoindole-1,3-dione | | 1H), 6.61 (dd, J = 8.4, 2.1 Hz, 1H), 6.50 (s, |
| | | | 1H), 5.05 (dd, J = 12.9, 5.5 Hz, 1H), 4.60 (s, |
| | | | 1H), 4.45 (t, J = 6.7 Hz, 2H), 4.10 (t, J = 8.0 |
| | | | Hz, 2H), 3.72 (s, 3H), 3.66-3.59 (m, 4H), 3.13 |
| | | | (t, J = 6.8 Hz, 2H), 2.89-2.80 (m, 1H), 2.74- |
| | | | 2.66 (m, 1H), 2.60 (s, 1H), 2.03-1.95 (m, 1H), |
| | | | 1.69-1.57 (m, 2H), 1.53-1.39 (m, 2H). |
| 124 | 4-((5-((4-(2-((3-amino-6-(3- | 713.4 | ¹H NMR (300 MHz, DMSO-d6) δ 15.83 (s, 1H), |
| | chloro-2-hydroxyphenyl) | | 11.12 (s, 1H), 8.83 (s, 1H), 7.96 (dd, J= 8.2, |
| | pyridazin-4-yl)oxy)ethyl) | | 1.6 Hz, 1H), 7.82 (t, J= 7.9 Hz, 1H), 7.66 (s, |
| | benzyl)amino)pentyl)oxy)- | | 1H), 7.56-7.35 (m, 7H), 6.88 (t, J = 7.9 Hz, |
| | 2-(2,6-dioxopiperidin-3-yl) | | 1H), 6.68 (s, 2H), 5.08 (dd, J= 12.9, 5.3 Hz, |
| | isoindoline-1,3-dione | | 1H), 4.49 (t, J = 6.6 Hz, 2H), 4.21 (t, J = 6.1 |
| | | | Hz, 2H), 4.13 (s, 2H), 3.18 (t, J= 6.6 Hz, 2H), |
| | | | 3.00-2.81 (m, 3H), 2.76-2.59 (m, 2H), 2.09- |
| | | | 1.96 (m, 1H), 1.88-1.60 (m, 4H), 1.59- |
| | | | 1.41 (m, 2H). |
| 118 | 4-[[5-([[4-(2-[[3-amino-6-(5- | 697.4 | ¹H NMR (400 MHz, DMSO-d6) δ 14.20 (s, 1H), |
| | fluoro-2-hydroxyphenyl) | | 11.10 (s, 1H), 8.29 (s, 1H, FA), 7.93-7.75 (m, |
| | pyridazin-4-yl]oxy]ethyl) | | 2H), 7.62 (s, 1H), 7.51 (d, J = 8.5 Hz, 1H), 7.45 |
| | phenyl]methyl]amino) | | (d, J = 7.3 Hz, 1H), 7.35 (q, J = 7.8 Hz, 4H), |
| | pentyl]oxy]-2-(2,6- | | 7.12-7.03 (m, 1H), 6.92-6.84 (m, 1H), 6.58 |
| | dioxopiperidin-3-yl)isoindole- | | (s, 2H), 5.08 (dd, J = 12.8, 5.4 Hz, 1H), 4.45 (t, |
| | 1,3-dione;formic acid salt | | J = 6.9 Hz, 2H), 4.20 (t, J = 6.4 Hz, 2H), 3.78 |
| | | | (s, 2H), 3.14 (t, J = 6.9 Hz, 2H), 2.91-2.81 (m, |
| | | | 1H), 2.66-2.54 (m, 4H), 2.07-1.96 (m, 1H), |
| | | | 1.85-1.65 (m, 2H), 1.65-1.37 (m, 4H). |
| 119 | 4-((5-((4-(2-((3-amino-6-(3- | 731.4 | ¹H NMR (400 MHz, DMSO-d6) δ 11.08 (s, 1H), |
| | chloro-5-fluoro-2- | | 8.27 (s, 1H, FA), 7.94 (dd, J= 10.5, 3.0 Hz, |
| | hydroxyphenyl)pyridazin-4- | | 1H), 7.80 (t, J = 7.9 Hz, 1H), 7.67 (s, 1H), 7.50 |
| | yl)oxy)ethyl)benzyl)amino) | | (d, J = 8.5 Hz, 1H), 7.44 (d, J = 7.2 Hz, 1H), |
| | pentyl)oxy)-2-(2,6- | | 7.41-7.25 (m, 5H), 6.74 (s, 2H), 5.07 (dd, J = |
| | dioxopiperidin-3-yl) | | 12.8, 5.4 Hz, 1H), 4.46 (t, J = 6.9 Hz, 2H), 4.19 |
| | isoindoline-1,3-dione; | | (t, J = 6.3 Hz, 2H), 3.77 (s, 2H), 3.14 (t, J = 6.9 |
| | formic acid salt | | Hz, 2H), 2.92-2.82 (m, 2H), 2.63-2.55 (m, |
| | | | 3H), 2.06-1.97 (m, 1H), 1.80-1.71 (m, 2H), |
| | | | 1.59-1.43 (m, 4H). |
| 150 | 4-((5-((4-(2-((3-amino-6-(2- | 692.3 | ¹H NMR (300 MHz, DMSO-d6) δ 11.11 (s, 1H), |
| | hydroxyphenyl)pyridazin-4- | | 8.14 (s, 1H), 7.88-7.76 (m, 2H), 7.48 (dd, J = |
| | yl)amino)ethyl)benzyl)(methyl) | | 19.5, 7.8 Hz, 2H), 7.33 (s, 4H), 7.20 (t, J = 7.9 |
| | amino)pentyl)oxy)-2-(2,6- | | Hz, 1H), 6.97 (s, 1H), 6.83 (dt, J = 7.3, 3.1 Hz, |
| | dioxopiperidin-3-yl) | | 2H), 6.54 (s, 1H), 6.40 (s, 1H), 6.31 (s, 2H), |
| | isoindoline-1,3-dione, | | 5.07 (dd, J = 12.8, 5.4 Hz, 1H), 4.21 (t, J = 6.3 |
| | formic acid salt | | Hz, 2H), 3.80 (s, 2H), 3.54 (dd, J = 12.6, 6.2 |
| | | | Hz, 4H), 2.96 (t, J = 7.2 Hz, 2H), 2.92-2.78 |
| | | | (m, 1H), 2.73 (d, J = 2.3 Hz, 3H), 2.63 (d, J = |
| | | | 12.5 Hz, 2H), 2.31 (s, 3H), 2.02 (dd, J= 10.5, |
| | | | 5.1 Hz, 1H), 1.76 (q, J = 6.8 Hz, 2H), 1.65 (d, J |
| | | | = 8.7 Hz, 2H), 1.47 (q, J = 7.6 Hz, 2H). |
| 145 | 4-((5-((4-(2-((3-amino-6-(2- | 706.2 | ¹H NMR (400 MHz, DMSO-d6) δ 11.11 (s, 1H), |
| | hydroxyphenyl)pyridazin-4- | | 8.23 (s, 1H, FA), 7.89-7.75 (m, 2H), 7.54- |
| | yl)(methyl)amino)ethyl)benzyl) | | 7.31 (m, 3H), 7.26-7.18 (m, 1H), 7.14 (s, 4H), |
| | (methyl)amino)pentyl)oxy)-2- | | 6.92-6.83 (m, 2H), 6.08 (s, 2H), 5.08 (dd, J = |
| | (2,6-dioxopiperidin-3- | | 12.8, 5.4 Hz, 1H), 4.19 (t, J = 6.4 Hz, 2H), 3.31 |
| | yl)isoindoline-1,3- | | (s, 4H), 2.93 (s, 3H), 2.91-2.75 (m, 3H), 2.65- |
| | dione formate | | 2.52 (m, 2H), 2.27 (t, J = 6.7 Hz, 2H), 2.01 (s, |
| | | | 4H), 1.80-1.69 (m, 2H), 1.49-1.43 (m, 4H). |
| 148 | 5-((5-((4-(2-((3-amino-6-(2- | 692.9 | ¹H NMR (400 MHz, DMSO-d₆) 14.40 (s, 1H), |
| | hydroxyphenyl)pyridazin-4- | | 11.11 (s, 1H), 8.22 (s, 1H), 7.93 (dd, J = 8.0, |
| | yl)oxy)ethyl)benzyl)(methyl)ami | | 1.6 Hz, 1H), 7.82 (d, J = 8.3 Hz, 1H), 7.59 (s, |
| | no)pentyl)oxy)-2-(2,6- | | 1H), 7.42 (d, J = 2.3 Hz, 1H), 7.39-7.30 (m, |
| | dioxopiperidin-3-yl)isoindoline- | | 3H), 7.27-7.18 (m, 3H), 6.91-6.81 (m, 2H), |
| | 1,3-dioneformate | | 6.50 (s, 2H), 5.16-5.07 (m, 1H), 4.45 (t, J = |
| | | | 7.0 Hz, 2H), 4.16 (t, J = 6.5 Hz, 2H), 3.42 (s, |
| | | | 2H), 3.12 (t, J = 6.9 Hz, 2H), 2.95-2.82 (m, |
| | | | 1H), 2.64-2.52 (m, 2H), 2.32 (t, J = 7.0 Hz, |
| | | | 2H), 2.12-1.98 (m, 4H), 1.80-1.68 (m, 2H), |
| | | | 1.58-1.42 (m, 2H), 1.47-1.38 (m, 2H). |
| 153 | 4-(4-(2-((4-(2-((3-amino-6-(2- | 719.4 | ¹H NMR (400 MHz, DMSO-d6) δ 14.21 (s, 1H), |
| | hydroxyphenyl)pyridazin-4- | | 11.01 (s, 1H), 8.06 (s, 1H), 7.84 (d, J= 8.0 Hz, |
| | yl)oxy)ethyl)benzyl)amino)ethyl | | 1H), 7.62 (t, J = 7.8 Hz, 1H), 7.50 (s, 1H), 7.33- |
| | )-3-oxopiperazin-1-yl)-2-(2,6- | | 7.23 (m, 6H), 7.14 (t, J= 7.8 Hz, 1H), 6.77 (t, |
| | dioxopiperidin-3-yl)isoindoline- | | J = 7.9 Hz, 2H), 6.45 (s, 1H), 6.40 (s, 2H), 5.01 |
| | 1,3-dione | | (dd, J = 12.8, 5.4 Hz, 1H), 4.36 (t, J = 6.9 Hz, |
| | | | 2H), 3.87 (s, 2H), 3.79 (s, 2H), 3.60 (s, 3H), |
| | | | 3.54 (s, 1H), 3.47 (q, J = 7.3, 6.7 Hz, 4H), 3.05 |
| | | | (d, J = 6.7 Hz, 2H), 2.78 (s, 3H), 2.53 (dd, J = |
| | | | 22.8, 16.6 Hz, 2H), 2.45 (s, 1H), 1.94 (d, J = |
| | | | 12.8 Hz, 1H). |
| 191 | (2S,4R)-1-[(2R)-2-[3-(2-[6- | 999.0 | ¹H NMR (400 MHz, DMSO-d6) δ 14.38 (s, 1H), |
| | [([[4-(2-[[3-amino-6-(2- | | 8.98 (s, 1H), 8.41 (d, J = 7.7 Hz, 1H), 8.13 (t, J = |
| | hydroxyphenyl)pyridazin-4- | | 6.2 Hz, 1H), 7.95 (d, J = 7.7 Hz, 1H), 7.59 (s, |
| | yl]oxy]ethyl)phenyl]methyl] | | 1H), 7.43 (d, J = 8.4 Hz, 2H), 7.39-7.31 (m, |
| | carbamoyl)methyl]-2,6- | | 4H), 7.27-7.19 (m, 1H), 7.18 (d, J = 7.9 Hz, |
| | diazaspiro[3.3]heptan-2-yl] | | 2H), 6.91-6.82 (m, 2H), 6.50 (s, 2H), 6.05 (s, |
| | ethoxy)-1,2-oxazol-5-yl]-3- | | 1H), 5.10 (d, J = 3.7 Hz, 1H), 4.91 (q, J = 7.0 |
| | methylbutanoyl]-4-hydroxy-N- | | Hz, 1H), 4.40 (dt, J = 26.7, 7.4 Hz, 3H), 4.32- |
| | [(1S)-1-[4-(4-methyl-1,3-thiazol- | | 4.17 (m, 3H), 4.09-3.56 (m, 4H), 3.23 (s, 4H), |
| | 5-yl)phenyl]ethyl]pyrrolidine-2- | | 3.11 (t, J = 6.8 Hz, 2H), 3.00 (s, 2H), 2.64 (s, |
| | carboxamide | | 0H), 2.45 (s, 3H), 2.37-2.13 (m, 2H), 2.01 (d, |
| | | | J = 10.4 Hz, 1H), 1.44 (d, J = 7.1 Hz, 1H), 1.37 |
| | | | (d, J = 7.0 Hz, 3H), 0.96 (t, J = 6.3 Hz, 3H), |
| | | | 0.80 (, J = 14.5, 6.7 Hz, 3H). |
| 192 | (2S,4R)-1-[(2R)-2-[3-(2-[6-[2- | 1013.4 | ¹H NMR (400 MHz, DMSO-d₆) δ 14.37 (s, 1H), |
| | ([[4-(2-[[3-amino-6-(2- | | 8.99 (d, J = 2.1 Hz, 1H), 8.41 (d, J = 7.7 Hz, |
| | hydroxyphenyl)pyridazin-4- | | 1H), 8.35 (s, 1H), 7.97-7.91 (m, 1H), 7.59 (s, |
| | yl]oxy]ethyl)phenyl]methyl] | | 1H), 7.49-7.40 (m, 2H), 7.40-7.31 (m, 4H), |
| | carbamoyl)ethyl]-2,6- | | 7.27-7.18 (m, 3H), 6.87 (t, J= 7.5 Hz, 2H), |
| | diazaspiro[3.3]heptan-2-yl] | | 6.50 (s, 2H), 6.05 (s, 1H), 5.10 (s, 1H), 4.91 (p, |
| | ethoxy)-1,2-oxazol-5-yl]-3- | | J = 6.7 Hz, 1H), 4.43 (t, J = 6.8 Hz, 2H), 4.36 |
| | methylbutanoyl]-4-hydroxy-N- | | (t, J = 7.9 Hz, 1H), 4.30-4.20 (m, 3H), 4.05 (t, |
| | [(1S)-1-[4-(4-methyl-1,3-thiazol- | | J = 5.2 Hz, 2H), 3.73-3.60 (m, 2H), 3.43 (d, J |
| | 5-yl)phenyl]ethyl]pyrrolidine-2- | | = 10.7 Hz, 1H), 3.26-3.09 (m, 9H), 2.45 (s, |
| | carboxamide | | 3H), 2.60-2.51 (m, 2H), 2.48-2.37 (m, 4H), |
| | | | 2.28-2.17 (m, 1H), 2.13 (s, 2H), 2.07-1.99 |
| | | | (m, 1H), 1.78 (ddd, J= 12.8, 8.1,4.8 Hz, 1H), |
| | | | 1.44 (d, J = 7.0 Hz, 1H), 1.37 (d, J = 7.0 Hz, |
| | | | 2H), 0.96 (t, J = 6.3 Hz, 3H), 0.80 (dd, J = |
| | | | 14.5, 6.7 Hz, 3H). |
| 174 | (2S,4R)-1-((R)-2-(3-(2-(4-(2- | 987.5 | ¹H NMR (400 MHz, DMSO-d⁶) δ 14.38 (s, 1H), |
| | ((4-(2-((3-amino-6-(2- | | 8.98 (s, 1H), 8.41 (d, J= 7.7 Hz, 1H), 8.20 (s, |
| | hydroxyphenyl)pyridazin-4- | | 1H), 7.98-7.89 (m, 1H), 7.59 (s, 1H), 7.49- |
| | yl)oxy)ethyl)benzyl)amino)-2- | | 7.40 (m, 2H), 7.40-7.29 (m, 4H), 7.26-7.18 |
| | oxoethyl)piperazin-1- | | (m, 3H), 6.92-6.82 (m, 2H), 6.50 (s, 2H), 6.09 |
| | yl)ethoxy)isoxazol-5-yl)-3- | | (s, 1H), 5.23-4.73 (m, 2H), 4.40 (dt, J = 28.1, |
| | methylbutanoyl)-4-hydroxy-N- | | 7.4 Hz, 3H), 4.28-4.22 (m, 5H), 3.72-3.60 |
| | ((S)-1-(4-(4-methylthiazol-5- | | (m, 2H), 3.44 (d, J= 10.9 Hz, 1H), 3.12 (t, J = |
| | yl)phenyl)ethyl)pyrrolidine-2- | | 6.8 Hz, 2H), 2.95 (s, 2H), 2.67 (s, 2H), 2.50- |
| | carboxamide | | 2.38 (m, 11H), 2.39-2.11 (m, 1H), 2.06-1.90 |
| | | | (m, 1H), 1.78 (ddd, J = 12.6, 8.1,4.9 Hz, 1H), |
| | | | 1.41 (dd, J = 29.3, 7.0 Hz, 3H), 0.96 (t, J = 6.4 |
| | | | Hz, 3H), 0.81 (dd, J = 14.2, 6.7 Hz, 3H). |
| 175 | (2S,4R)-1-[(2R)-2-[3-(2-[4-[2- | 1001.40 | ¹H NMR (400 MHz, DMSO-d⁶) δ 14.37 (s, 1H), |
| | ([[4-(2-[[3-amino-6-(2- | | 8.99 (d, J = 1.6 Hz, 1H), 8.41 (d, J = 7.8 Hz, |
| | hydroxyphenyl)pyridazin-4- | | 2H), 7.94 (d, J= 8.2 Hz, 1H), 7.59 (s, 1H), 7.49- |
| | yl]oxy]ethyl)phenyl]methyl] | | 7.40 (m, 2H), 7.40-7.32 (m, 4H), 7.27- |
| | carbamoyl)ethyl]piperazin-1- | | 7.18 (m, 3H), 6.87 (d, J = 8.0 Hz, 2H), 6.49 (s, |
| | yl]ethoxy)-1,2-oxazol-5-yl]-3- | | 2H), 6.08 (s, 1H), 5.10 (d, J= 3.7 Hz, 1H), 4.91 |
| | methylbutanoyl]-4-hydroxy-N- | | (q, J = 7.5 Hz, 1H), 4.43-4.36 (m, 3H), 4.28- |
| | [(1S)-1-[4-(4-methyl-1,3-thiazol- | | 4.20 (m, 5H), 3.74-3.60 (m, 2H), 3.44 (d, J = |
| | 5-yl)phenyl]ethyl]pyrrolidine-2- | | 10.6 Hz, 1H), 3.12 (t, J = 6.8 Hz, 2H), 2.70- |
| | carboxamide | | 2.62 (m, 2H), 2.45 (s, 3H), 2.44-2.33 (m, |
| | | | 4H), 2.30-2.25 (m, 4H), 2.02 (s, 1H), 1.83- |
| | | | 1.72 (m, 1H), 1.37 (d, J = 7.0 Hz, 3H), 0.96 (t, |
| | | | J = 6.4 Hz, 3H), 0.81 (dd, J = 14.9, 6.6 Hz, 3H). |
| 176 | N-(4-(2-((3-amino-6-(2- | 972.2 | ¹H NMR (400 MHz, DMSO-d⁶) δ 14.38 (s, 1H), |
| | hydroxyphenyl)pyridazin-4- | | 8.98 (s, 1H), 8.40 (t, J = 10.2 Hz, 1H), 8.26 (d, |
| | yl)oxy)ethyl)benzyl)-1-(2-((5- | | J = 6.4 Hz, 1H), 7.94 (d, J = 7.9 Hz, 1H), 7.59 |
| | ((R)-1-((2S,4R)-4-hydroxy-2- | | (s, 1H), 7.44 (d, J = 8.3 Hz, 2H), 7.36 (q, J = |
| | (((S)-1-(4-(4-methylthiazol-5- | | 7.5, 6.1 Hz, 4H), 7.20 (dd, J = 16.3, 7.7 Hz, |
| | yl)phenyl)ethyl)carbamoyl)pyrro | | 3H), 6.88 (d, J = 8.0 Hz, 2H), 6.50 (s, 2H), 6.09 |
| | lidin-1-yl)-3-methyl-1-oxobutan- | | (s, 1H), 5.10 (s, 1H), 4.99-4.83 (m, 1H), 4.40 |
| | 2-yl)isoxazol-3-yl) | | (dt, J = 25.4, 7.5 Hz, 3H), 4.32-4.13 (m, 5H), |
| | oxy)ethyl)piperidine-4- | | 3.79-3.60 (m, 1H), 3.51 (d, J = 23.9 Hz, 1H), |
| | carboxamide | | 3.47-3.42 (m, 1H), 3.12 (t, J = 7.0 Hz, 2H), |
| | | | 2.91 (d, J= 10.8 Hz, 2H), 2.65 (t, J= 5.9 Hz, |
| | | | 2H), 2.45 (s, 3H), 2.24 (s, 1H), 2.13 (s, 1H), |
| | | | 1.99 (t, J = 11.8 Hz, 3H), 1.79 (dd, J = 8.6, 4.9 |
| | | | Hz, 1H), 1.70-1.56 (m, 4H), 1.37 (d, J = 7.0 |
| | | | Hz, 3H), 0.95 (d, J = 6.6 Hz, 3H), 0.81 (t, J = |
| | | | 8.9 Hz, 3H). |
| 177 | (2S,4R)-1-((R)-2-(3-(2-(4-(2- | 986.5 | ¹H NMR (300 MHz, DMSO-d⁶) δ 9.06 (s, 1H), |
| | ((4-(2-((3-amino-6-(2- | | 8.57-8.23 (m, 3H), 8.12-7.98 (m, 1H), 7.67 |
| | hydroxyphenyl)pyridazin-4- | | (s, 1H), 7.58-7.38 (m, 6H), 7.28 (t, J = 7.5 Hz, |
| | yl)oxy)ethyl)benzyl)amino)- | | 3H), 7.05-6.89 (m, 2H), 6.58 (s, 2H), 6.17 (s, |
| | 2-oxoethyl)piperidin-1- | | 1H), 4.99 (q, J = 7.1 Hz, 1H), 4.56-4.39 (m, |
| | yl)ethoxy)isoxazol-5-yl)-3- | | 3H), 4.39-4.23 (m, 6H), 3.81-3.67 (m, 3H), |
| | methylbutanoyl)-4-hydroxy-N- | | 3.19 (t, J = 6.9 Hz, 2H), 2.93 (d, J = 11.0 Hz, |
| | ((S)-1-(4-(4-methylthiazol-5- | | 2H), 2.71 (t, J = 5.6 Hz, 2H), 2.53 (s, 3H), 2.30 |
| | yl)phenyl)ethyl)pyrrolidine-2- | | (s, 2H), 2.16-1.98 (m, 5H), 1.92-1.78 (m, |
| | carboxamide | | 1H), 1.66 (d, J = 13.0 Hz, 2H), 1.49 (dd, J = |
| | | | 22.1,7.0 Hz, 3H), 1.35-1.15 (m, 2H), 1.03 (d, |
| | | | J = 6.3 Hz, 3H), 0.88 (dd, J = 10.9, 6.6 Hz, 3H), |
| | | | 0.07 (s, 1H). |
| 178 | (2S,4R)-1-((R)-2-(3-(2-(3- | 944.15 | ¹H NMR (400 MHz, DMSO-d₆) δ 9.01-8.96 |
| | ((4-(2-((3-amino-6-(2- | | (m, 1H), 8.44-8.32 (m, 1H), 8.20-8.15 (m, |
| | hydroxyphenyl)pyridazin-4- | | 2H), 8.13 (s, 1H), 7.98-7.91 (m, 1H), 7.62- |
| | yl)oxy)ethyl)benzyl)carbamoyl) | | 7.57 (m, 1H), 7.50-7.40 (m, 2H), 7.40-7.31 |
| | azetidin-1-yl)ethoxy)isoxazol- | | (m, 4H), 7.28-7.16 (m, 3H), 6.91-6.82 (m, |
| | 5-yl)-3-methylbutanoyl)-4- | | 2H), 6.53-6.49 (m, 2H), 6.06 (s, 1H), 4.96- |
| | hydroxy-N-((S)-1-(4-(4- | | 4.86 (m, 1H), 4.47-4.40 (m, 2H), 4.40-4.32 |
| | methylthiazol-5-yl)phenyl) | | (m, 1H), 4.32-4.20 (m, 4H), 4.12-4.04 (m, |
| | ethyl)pyrrolidine-2- | | 1H), 3.72-3.60 (m, 4H), 3.47-3.40 (m, 2H), |
| | carboxamide | | 3.21-3.17 (m, 2H), 3.14-3.10 (m, 2H), 2.75- |
| | | | 2.69 (m, 1H), 2.45 (s, 3H), 2.24-2.20 (m, |
| | | | 2H), 2.07-1.98 (m, 1H), 1.82-1.73 (m, 1H), |
| | | | 1.47-1.35 (m, 3H), 0.99-0.92 (m, 3H), 0.86- |
| | | | 0.76 (m, 3H). |
| 193 | (2S,4R)-1-[2-[3-(2-[3-[([[4-(2- | 958.30 | ¹H NMR (400 MHz, DMSO-d₆) δ 14.38 (s, 1H), |
| | [[3-amino-6-(2- | | 8.99 (d, J = 1.9 Hz, 1H), 8.41 (d, J = 7.6 Hz, |
| | hydroxyphenyl)pyridazin-4- | | 1H), 8.35 (s, 1H), 7.94 (d, J = 7.8 Hz, 1H), 7.59 |
| | yl]oxy]ethyl)phenyl]methyl] | | (s, 1H), 7.49-7.40 (m, 2H), 7.39-7.29 (m, |
| | carbamoyl)methyl]azetidin-1- | | 4H), 7.28-7.15 (m, 3H), 6.87 (t, J = 7.9 Hz, |
| | yl]ethoxy)-1,2-oxazol-5-yl]-3- | | 2H), 6.50 (s, 2H), 6.08 (s, 1H), 5.10 (d, J= 3.6 |
| | methylbutanoyl]-4-hydroxy-N- | | Hz, 1H), 4.95-4.87 (m, 1H), 4.47-4.32 (m, |
| | [(1S)-1-[4-(4-methyl-1,3-thiazol- | | 3H), 4.28 (s, 1H), 4.22 (d, J = 5.9 Hz, 2H), |
| | 5-yl)phenyl]ethyl]pyrrolidine- | | 4.19-4.08 (m, 2H), 3.73-3.61 (m, 2H), 3.59- |
| | 2-carboxamide | | 3.50 (m, 1H), 3.48-3.42 (m, 1H), 2.45 (s, |
| | | | 3H), 2.44-2.37 (m, 2H), 2.31-2.13 (m, 2H), |
| | | | 2.02 (t, J= 10.0 Hz, 1H), 1.78 (dd, J= 10.9, 6.1 |
| | | | Hz, 1H), 1.47-1.33 (m, 3H), 1.30-1.19 (m, |
| | | | 1H), 1.17-1.09 (m, 1H), 0.96 (t, J = 6.1 Hz, |
| | | | 3H), 0.81 (dd, J = 14.2, 6.6 Hz, 3H). |
| 205 | (2S,4R)-1-[(2R)-2-[3-[2- | 861.35 | ¹H NMR (400 MHz, DMSO-d6) δ 14.39 (s, 1H), |
| | ([[4-(2-[[3-amino-6-(2- | | 8.99 (s, 1H), 8.42 (d, J = 7.7 Hz, 1H), 7.98- |
| | hydroxyphenyl)pyridazin-4- | | 7.91 (m, 1H), 7.60 (s, 1H), 7.49-7.40 (m, 2H), |
| | yl]oxy]ethyl)phenyl]methyl] | | 7.40-7.23 (m, 7H), 6.87 (t, J = 7.6 Hz, 2H), |
| | amino)ethoxy]-1,2-oxazol- | | 6.52 (s, 2H), 6.08 (s, 1H), 5.10 (d, J = 3.6 Hz, |
| | 5-yl]-3-methylbutanoyl]-4- | | 1H), 4.91 (p, J = 7.3 Hz, 1H), 4.45 (t, J = 6.9 |
| | hydroxy-N-[(1S)-1-[4-(4- | | Hz, 2H), 4.36 (t, J = 7.9 Hz, 1H), 4.28 (s, 1H), |
| | methyl-1,3-thiazol-5-yl)phenyl] | | 4.24-4.16 (m, 2H), 3.76-3.61 (m, 4H), 3.44 |
| | ethyl]pyrrolidine-2-carboxamide | | (d, J = 10.8 Hz, 1H), 3.13 (t, J = 6.9 Hz, 2H), |
| | | | 2.85 (s, 2H), 2.45 (s, 3H), 2.29-2.14 (m, 1H), |
| | | | 2.07-1.98 (m, 1H), 1.83-1.72 (m, 1H), 1.41 |
| | | | (d, J = 29.9, 7.0 Hz, 3H), 0.95 (d, J = 6.5 Hz, |
| | | | 3H), 0.81 (d, J = 14.6, 6.7 Hz, 3H). |
| 194 | 1R,5S,6S)-N-[[4-(2-[[3-amino- | 970.40 | ¹H NMR (400 MHz, DMSO-d⁶) δ 14.39 (s, 1H), |
| | 6-(2-hydroxyphenyl)pyridazin- | | 8.99 (s, 1H), 8.50 (t, J = 6.1 Hz, 1H), 8.42 (d, J = |
| | 4-yl]oxy]ethyl)phenyl]methyl]- | | 7.6 Hz, 1H), 7.95 (d, J = 7.9 Hz, 1H), 7.60 (s, |
| | 3-[2-([5-[(2R)-1-[(2S,4R)-4- | | 1H), 7.49-7.40 (m, 2H), 7.36 (dt, J = 7.9, 4.3 |
| | hydroxy-2-[[(1S)-1-[4-(4- | | Hz, 4H), 7.27-7.15 (m, 3H), 6.87 (t, J = 7.8 Hz, |
| | methyl-1,3-thiazol-5-yl)phenyl] | | 2H), 6.51 (s, 2H), 6.08 (s, 1H), 5.14-5.08 (m, |
| | ethyl]carbamoyl]pyrrolidin-1- | | 1H), 4.91 (q, J = 6.8 Hz, 1H), 4.39 (dd, J = |
| | yl]-3-methyl-1-oxobutan- | | 28.1,7.4 Hz, 3H), 4.25-4.08 (m, 4H), 3.76- |
| | 2-yl]-1,2-oxazol-3-yl]oxy)ethyl]- | | 3.60 (m, 2H), 3.59-3.41 (m, 2H), 3.18-2.99 (m, |
| | 3-azabicyclo[3.1.0]hexane-6- | | 4H), 2.80-2.65 (m, 2H), 2.41 (d, J= 8.4 Hz, |
| | carboxamide | | 3H), 2.33 (q, J = 1.9 Hz, 2H), 2.30-2.11 (m, |
| | | | 1H), 2.04 (dd, J = 20.9, 10.0 Hz, 1H), 1.93 (t, J = |
| | | | 2.8 Hz, 1H), 1.81-1.70 (m, 3H), 1.37 (d, J = |
| | | | 7.0 Hz, 3H), 0.96 (t, J = 6.2 Hz, 3H), 0.81 (dd, |
| | | | J = 14.0, 6.6 Hz, 3H). |
| 206 | N-[[4-(2-[[3-amino-6-(2- | | ¹H NMR (400 MHz, DMSO-d6) δ 14.38 (s, 1H), |
| | hydroxyphenyl)pyridazin-4- | | 8.99 (s, 1H), 8.42 (d, J= 7.5 Hz, 1H), 8.28 (s, |
| | yl]oxy]ethyl)phenyl]methyl]-4- | | 1H), 7.95 (d, J= 7.9 Hz, 1H), 7.60 (s, 1H), 7.44 |
| | hydroxy-1-[2-([5-[(2R)-1- | | (d, J = 8.2 Hz, 2H), 7.41-7.31 (m, 4H), 7.21 (t, |
| | [(2S,4R)-4-hydroxy-2-[[(1S)-1- | | J = 7.8 Hz, 3H), 6.87 (t, J = 8.0 Hz, 2H), 6.51 (s, |
| | [4-(4-methyl-1,3-thiazol-5- | | 2H), 6.11 (s, 1H), 5.30(s, 1H),5.11 (d, J = 3.6 |
| | yl)phenyl]ethyl]carbamoyl] | | Hz, 1H), 4.95-4.87 (m, 1H), 4.40 (dt, J = 26.7, |
| | pyrrolidin-1-yl]-3-methyl- | | 7.4 Hz, 3H), 4.26 (d, J = 6.4 Hz, 5H), 3.77-3.56 |
| | 1-oxobutan-2-yl]-1,2-oxazol-3- | | (m, 2H), 3.33 (d, J = 1.2 Hz, 3H), 3.12 (t, J = 6.8 |
| | yl]oxy)ethyl]piperidine-4- | | Hz, 3H), 2.50 (p, J = 1.9 Hz, 4H), 2.28 (d, J = |
| | carboxamide | | 38.6 Hz, 2H), 1.97 (dd, J = 34.7, 11.9 Hz, 3H), |
| | | | 1.78 (dt, J= 12.8, 5.7 Hz, 1H), 1.45 (d, J = 6.9 |
| | | | Hz, 2H), 1.38 (d, J= 7.0 Hz, 3H), 1.24 (s, 1H), |
| | | | 0.96 (d, J = 6.5 Hz, 3H), 0.82 (dd, J = 14.5, 6.6 |
| | | | Hz, 3H). |
| 195 | N-(4-(2-((3-amino-6-(2- | 1012.40 | ¹H NMR (300 MHz, DMSO-d6) δ 14.38 (s, 1H), |
| | hydroxyphenyl)pyridazin-4- | | 8.99 (d, J = 1.6 Hz, 1H), 8.42 (d, J = 7.7 Hz, 1H), |
| | yl)oxy)ethyl)benzyl)-7-(2-((5- | | 8.16 (t, J = 6.0 Hz, 1H), 7.98-7.92 (m, 1H), |
| | ((R)-1-((2S,4R)-4-hydroxy-2- | | 7.60 (s, 1H), 7.44 (d, J = 8.0 Hz, 2H), 7.40-7.31 |
| | (((S)-1-(4-(4-methylthiazol-5- | | (m, 4H), 7.27-7.16 (m, 3H), 6.87 (dd, J = 8.1, |
| | yl)phenyl)ethyl)carbamoyl) | | 6.8 Hz, 2H), 6.51 (s, 2H), 6.09 (s, 1H), 5.11 (d, |
| | pyrrolidin-1-yl)-3-methyl-1- | | J = 3.6 Hz, 1H), 4.97-4.86 (m, 1H), 4.47-4.33 |
| | oxobutan-2-yl)isoxazol-3-yl) | | (m, 3H), 4.32-4.26 (m, 1H), 4.25-4.16 (m, |
| | oxy)ethyl)-7-azaspiro[3.5] | | 4H), 3.75-3.40 (m, 3H), 3.12 (t, J = 6.8 Hz, 2H), |
| | nonane-2-carboxamide | | 3.02-2.94 (m, 1H), 2.66-2.58 (m, 2H), 2.46 |
| | | | (s, 3H), 2.41-2.18 (m, 5H), 2.07-1.76 (m, 7H), |
| | | | 1.59-1.52 (m, 2H), 1.50-1.44 (m, 2H), 1.38 |
| | | | (d, J = 7.0 Hz, 2H), 0.96 (d, J = 6.4 Hz, 3H), 0.80 |
| | | | (d, 3H). |
| 207 | (2S,4R)-1-[(2R)-2-[3-[2- | 998.60 | ¹H NMR (400 MHz, DMSO-d⁶) δ 14.36 (s, 1H), |
| | (4-[1-[4-(2-[[3-amino-6-(2- | | 8.98 (s, 1H), 8.41 (d, J = 7.6 Hz, 1H), 7.93 (d, |
| | hydroxyphenyl)pyridazin-4- | | J = 7.8 Hz, 1H), 7.59 (d, J = 8.3 Hz, 3H), 7.52- |
| | yl]oxy]ethyl)benzoyl]azetidin- | | 7.40 (m, 4H), 7.36 (d, J = 7.9 Hz, 2H), 7.23 (t, |
| | 3-yl]piperidin-1-yl)ethoxy]-1,2- | | J = 7.7 Hz, 1H), 6.91-6.83 (m, 2H), 6.52 (s, 2H), |
| | oxazol-5-yl]-3-methylbutanoyl]- | | 6.09 (s, 1H), 5.10 (d, J = 3.6 Hz, 1H), 4.96-4.85 |
| | 4-hydroxy-N-[(1S)-1-[4-(4- | | (m, 1H), 4.49 (t, J = 6.8 Hz, 2H), 4.40-4.14 (m, |
| | methyl-1,3-thiazol-5-yl) | | 5H), 4.10-3.92 (m, 2H), 3.79-3.59 (m, 3H), |
| | phenyl]ethyl]pyrrolidine-2- | | 3.59-3.40 (m, 2H), 3.19 (t, J = 6.7 Hz, 2H), |
| | carboxamide | | 2.97-2.78 (m, 2H), 2.72-2.58 (m, 1H), 2.45 |
| | | | (s, 3H), 2.39-2.14 (m, 2H), 2.07-1.85 (m, 3H), |
| | | | 1.83-1.72 (m, 1H), 1.69-1.50 (m, 2H), 1.49- |
| | | | 1.42 (m, 1H), 1.37 (d, J = 7.0 Hz, 3H), 1.13- |
| | | | 1.00 (m, 2H), 0.95 (d, J = 6.5 Hz, 3H), 0.81 |
| | | | (d, J = 6.5Hz, 3H). |
| 220 | (2S,4R)-1-((2R)-2-(3-(2-(8-(4- | 970.40 | ¹H NMR (300 MHz, DMSO-d6) δ 14.38 (s, 1H), |
| | (2-((3-amino-6-(2- | | 8.99 (s, 1H), 8.42 (d, J = 7.8 Hz, 1H), 7.99-7.90 |
| | hydroxyphenyl)pyridazin-4- | | (m, 1H), 7.61 (s, 1H), 7.52-7.32 (m, 8H), 7.27- |
| | yl)oxy)ethyl)benzoyl)-3,8- | | 7.19 (m, 1H), 6.92-6.84 (m, 2H), 6.54 (s, 2H), |
| | diazabicyclo[3.2.1]octan-3- | | 6.10 (s, 1H), 5.11 (d, J = 3.6 Hz, 1H), 4.91 (t, |
| | yl)ethoxy)isoxazol-5-yl)-3- | | J =7.1 Hz, 1H), 4.51 (t, J = 6.9 Hz, 3H), 4.42- |
| | methylbutanoyl)-4-hydroxy-N- | | 4.33 (m, 1H), 4.32-4.20 (m, 3H), 3.89 (s, 1H), |
| | ((S)-1-(4-(4-methylthiazol-5- | | 3.75-3.61 (m, 2H), 3.51-3.43 (m, 1H), 3.20 |
| | yl)phenyl)ethyl)pyrrolidine-2- | | (t, J = 6.8 Hz, 2H), 2.80-2.65 (m, 4H), 2.46 (s, |
| | carboxamide | | 3H), 2.42-2.36 (m, 1H), 2.30-2.21 (m, 2H), |
| | | | 2.09-1.98 (m, 1H), 1.84-1.67 (m, 5H), 1.38 |
| | | | (d, J = 7.0 Hz, 3H), 0.96 (d, J = 6.4 Hz, 3H), 0.80 |
| | | | (d, J = 6.7 Hz, 3H). |
| 208 | (2S,4R)-1-[(2R)-2-[3-(2-[6-[4- | 956.45 | ¹H NMR (400 MHz, DMSO-d6) δ 14.38 (s, 1H), |
| | (2-[[3-amino-6-(2- | | 8.99 (s, 1H), 8.42 (d, J = 7.7 Hz, 1H), 7.97-7.90 |
| | hydroxyphenyl)pyridazin-4- | | (m, 1H), 7.63-7.54 (m, 3H), 7.51-7.40 (m, |
| | yl]oxy]ethyl)benzoyl]-2,6- | | 4H), 7.40-7.33 (m, 2H), 7.28-7.19 (m, 1H), |
| | diazaspiro[3.3]heptan-2- | | 6.87 (td, J = 7.4, 1.2 Hz, 2H), 6.52 (s, 2H), 6.07 |
| | yl]ethoxy)-1,2-oxazol-5-yl]-3- | | (s, 1H),5.10(d, J = 3.6 Hz, 1H),4.91 (p, J = 6.9 |
| | methylbutanoyl]-4-hydroxy-N- | | Hz, 1H), 4.49 (t, J = 6.7 Hz, 2H), 4.40-4.23 (m, |
| | [(1S)-1-[4-(4-methyl-1,3-thiazol- | | 4H), 4.14-4.01 (m, 4H), 3.73-3.61 (m, 2H), |
| | 5-yl)phenyl]ethyl]pyrrolidine-2- | | 3.44 (d, J = 11.0 Hz, 1H), 3.31 (s, 4H), 3.20 (t, J |
| | carboxamide | | = 6.6 Hz, 2H), 2.69 (s, 2H), 2.45 (s, 3H), 2.29- |
| | | | 2.12 (m, 1H), 2.02 (t, J = 9.2 Hz, 1H), 1.83- |
| | | | 1.72 (m, 1H), 1.37 (d, J = 7.0 Hz, 3H), 0.96 (d, J |
| | | | = 6.3 Hz, 3H), 0.81 (d, J = 14.2, 6.7 Hz, 3H). |
| 209 | (2S,4R)-1-[(2R)-2-[3-(2-[9-[4- | 1012.40 | ¹H NMR (400 MHz, DMSO-d6) δ 14.5 (s,1H), |
| | (2-[[3-amino-6-(2- | | 8.99 (d, J = 3.3 Hz, 1H), 8.42 (d, J = 7.7 Hz, 1H), |
| | hydroxyphenyl)pyridazin-4- | | 8.14 (d, J = 1.6 Hz, 1H), 8.00-7.83 (m, 1H), |
| | yl]oxy]ethyl)benzoyl]-3,9- | | 7.59 (s, 1H), 7.45 (dd, J = 11.1,8.1 Hz, 4H), 7.34 |
| | diazaspiro[5.5]undecan-3- | | (dd, J= 19.5, 8.0 Hz, 4H), 7.28-7.19 (m, 1H), |
| | yl]ethoxy)-1,2-oxazol-5-yl]-3- | | 6.93-6.80 (m, 2H), 6.53 (s, 2H), 6.11 (s, 1H), |
| | methylbutanoyl]-4-hydroxy-N- | | 5.11 (d, J=3.6 Hz, 1H),4.92(q, J = 7.1 Hz, 1H), |
| | [(1S)-1-[4-(4-methyl-1,3-thiazol- | | 4.50 (t, J = 6.8 Hz, 2H), 4.39-4.21 (m, 4H), |
| | 5-yl)phenyl]ethyl]pyrrolidine-2- | | 3.77-3.48 (m, 5H), 3.18 (t, J = 6.7 Hz, 4H), |
| | carboxamide | | 2.82 (s, 2H), 2.70-2.50 (m, 4H), 2.46 (s, 3H), |
| | | | 2.30-2.11 (m, 1H), 2.09-1.95 (m, 1H), 1.82- |
| | | | 1.73 (m, 1H), 1.59-1.42 (m, 6H), 1.37 (d, J = |
| | | | 7.0 Hz, 5H), 0.96 (d, J = 6.5 Hz, 3H), 0.81 (dd, |
| | | | J = 14.6, 6.5 Hz, 3H). |
| 210 | (2S,4R)-1-((R)-2-(3-(2-(4-((1- | 1042.40 | ¹H NMR (300 MHz, DMSO-d6) δ 14.38 (s, 1H), |
| | (4-(2-((3-amino-6-(2- | | 8.99 (d, J = 1.6 Hz, 1H), 8.43 (d, J = 7.6 Hz, 1H), |
| | hydroxyphenyl)pyridazin-4- | | 7.94 (dd, J = 8.4, 1.6 Hz, 1H), 7.60 (s, 1H), 7.45 |
| | yl)oxy)ethyl)benzoyl)piperidin- | | (t, J = 8.2 Hz, 4H), 7.40-7.30 (m, 4H), 7.24 (td, |
| | 4-yl)oxy)piperidin-1- | | J = 7.6, 1.5 Hz, 1H), 6.92-6.84 (m, 2H), 6.53 |
| | yl)ethoxy)isoxazol-5-yl)-3- | | (s, 2H), 6.10 (s, 1H), 5.12 (d, J = 3.6 Hz, 1H), |
| | methylbutanoyl)-4-hydroxy-N- | | 4.97-4.86 (m, 1H), 4.50 (t, J = 6.8 Hz, 2H), |
| | ((S)-1-(4-(4-methylthiazol-5- | | 4.37 (t, J = 7.9 Hz, 1H), 4.28 (s, 1H), 4.22 (t, J = |
| | yl)phenyl)ethyl)pyrrolidine-2- | | 5.6 Hz, 2H), 3.96 (s, 1H), 3.73-3.60 (m, 3H), |
| | carboxamide | | 3.23-3.16 (m, 4H), 2.81-2.61 (m, 5H), 2.46 |
| | | | (s, 3H), 2.31-2.11 (m, 4H), 2.05-1.98 (m, 1H), |
| | | | 1.85-1.70 (m, 5H), 1.48-1.30 (m, 8H), 0.96 |
| | | | (d, J = 6.5 Hz, 3H), 0.81 (dd, J = 10.7, 6.6 Hz, |
| | | | 3H). |
| 190 | (2S,4R)-1-((2R)-2-(3-(2-(5-(4- | 970.15 | ¹H NMR (400 MHz, DMSO-d6) δ 14.38 (s, 1H), |
| | (2-((3-amino-6-(2-hydroxyphenyl) | | 8.99 (s, 1H), 8.42 (d, J = 7.6 Hz, 1H), 7.98-7.89 |
| | pyridazin-4-yl)oxy)ethyl) | | (m, 1H), 7.60 (s, 1H), 7.52-7.39 (m, 6H), 7.40- |
| | benzoyl)hexahydropyrrolo | | 7.32 (m, 2H), 7.29-7.16 (m, 1H), 6.87 (dd, J = |
| | [3,4-c]pyrrol-2(1H)-yl) | | 8.3, 6.5 Hz, 2H), 6.53 (s, 2H), 6.10 (s, 1H), |
| | ethoxy)isoxazol-5-yl)-3- | | 5.11 (d, J = 3.6 Hz, 1H), 4.90 (q, J = 7.3 Hz, 1H), |
| | methylbutanoyl)-4-hydroxy-N- | | 4.50 (t, J = 6.8 Hz, 2H), 4.37 (t, J = 7.9 Hz, 1H), |
| | ((S)-1-(4-(4-methylthiazol-5- | | 4.25 (d, J = 24.1 Hz, 3H), 3.79-3.61 (m, 3H), |
| | yl)phenyl)ethyl)pyrrolidine-2- | | 3.61-3.50 (m, 1H), 3.45 (d, J = 10.9 Hz, 2H), |
| | carboxamide | | 3.19 (t, J = 6.7 Hz, 4H), 2.70 (d, J = 25.5 Hz, |
| | | | 4H), 2.45 (s, 4H), 2.24 (dt, J = 9.6, 6.5 Hz, 2H), |
| | | | 2.02 (t, J = 10.5 Hz, 1H), 1.77 (ddd, J = 12.7, |
| | | | 8.0, 4.7 Hz, 1H), 1.41 (dd, J = 31.6, 7.0 Hz, 4H), |
| | | | 0.96 (t, J = 6.3 Hz, 3H), 0.79 (d, J = 6.6 Hz, 3H). |
| 211 | (2S,4R)-1-[(2R)-2-[3-[2-(3- | 997.45 | ¹H NMR (300 MHz, DMSO-d6) δ 14.45 (s, 1H), |
| | [1-[4-(2-[[3-amino-6-(2- | | 9.06 (d, J = 1.9 Hz, 1H), 8.49 (d, J = 7.6 Hz, 1H), |
| | hydroxyphenyl)pyridazin-4- | | 8.06-7.96 (m, 1H), 7.67 (s, 1H), 7.52 (t, J = 7.8 |
| | yl]oxy]ethyl)benzoyl]piperidin-4- | | Hz, 4H), 7.47-7.35 (m, 4H), 7.36-7.25 (m, |
| | yl]azetidin-1-yl)ethoxy]-1,2- | | 1H), 7.00-6.89 (m, 2H), 6.60 (s, 2H), 6.14 (s, |
| | oxazol-5-yl]-3-methylbutanoyl]- | | 1H), 5.18 (d, J= 3.6 Hz, 1H), 4.98 (t, J= 7.1 Hz, |
| | 4-hydroxy-N-[(1S)-1-[4-(4- | | 1H), 4.57 (t, J = 6.9 Hz, 2H), 4.44 (t, J = 7.8 Hz, |
| | methyl-1,3-thiazol-5- | | 1H), 4.35 (s, 1H), 4.15 (s, 2H), 3.93-3.68 (m, |
| | yl)phenyl]ethyl]pyrrolidine-2- | | 2H), 3.65-3.44 (m, 2H), 3.24 (d, J = 6.8 Hz, |
| | carboxamide | | 2H), 2.94 (s, 4H), 2.79 (s, 3H), 2.53 (s, 3H), |
| | | | 2.39-1.91 (m, 4H), 1.85 (ddd, J = 12.9, 8.1,4.7 Hz, |
| | | | 1H), 1.70 (s, 2H), 1.52 (d, J = 6.9 Hz, 1H), 1.45 |
| | | | (d, J = 7.0 Hz, 3H), 1.03 (d, J = 6.4 Hz, 4H), 0.86 |
| | | | (d, J = 6.7 Hz, 3H). |
| 221 | (2S,4R)-1-[(2R)-2-[3-(2-[3-[4- | | ¹H NMR (300 MHz, DMSO-d6) δ 14.39 (s, 1H), |
| | (2-[[3-amino-6-(2- | | 8.99 (s, 1H), 8.42 (d, J = 7.7 Hz, 1H), 7.94 (d, |
| | hydroxyphenyl)pyridazin-4- | | J = 7.9 Hz, 1H), 7.61 (s, 1H), 7.46 (t, J = 8.0 Hz, |
| | yl]oxy]ethyl)benzoyl]-3,8- | | 4H), 7.37 (d, J = 8.3 Hz, 2H), 7.31 (d, J = 7.7 Hz, |
| | diazabicyclo[3.2.1]octan-8- | | 2H), 7.24 (t, J = 7.7 Hz, 1H), 6.88 (d, J = 7.7 Hz, |
| | yl]ethoxy)-1,2-oxazol-5-yl]-3- | | 2H), 6.54 (s, 2H), 6.11 (s, 1H), 5.11 (d, J = 3.7 |
| | methylbutanoyl]-4-hydroxy-N- | | Hz, 1H), 4.91 (t, J = 7.1 Hz, 1H), 4.51 (t, J = 6.9 |
| | [(1S)-1-[4-(4-methyl-1,3-thiazol- | | Hz, 2H), 4.37 (t, J = 7.7 Hz, 1H), 4.25 (d, J = |
| | 5-yl)phenyl]ethyl]pyrrolidine-2- | | 23.4 Hz, 4H), 3.76-3.39 (m, 3H), 3.28-3.03 |
| | carboxamide | | (m, 6H), 2.92 (s, 1H), 2.65 (s, 2H), 2.46 (s, 3H), |
| | | | 2.24 (s, 1H), 2.03 (s, 1H), 1.81 (s, 3H), 1.54 (s, |
| | | | 1H), 1.42 (dd, J = 23.3, 7.0 Hz, 4H), 0.96 (d, J = |
| | | | 6.4 Hz, 3H), 0.80 (d, J = 6.7 Hz, 3H). |
| 239 | (2S,4R)-1-((R)-2-(3-(2-((9-((4- | 1016.45 | ¹H NMR (300 MHz, DMSO-d6) δ 14.38 (s, 1H), |
| | (2-((3-amino-6-(2- | | 8.99 (d, J = 1.8 Hz, 1H), 8.42 (d, J = 7.6 Hz, 1H), |
| | hydroxyphenyl)pyridazin-4- | | 8.28 (d, J = 6.2 Hz, 1H), 7.95 (d, J = 7.8 Hz, 1H), |
| | yl)oxy)ethyl)benzyl)amino)-9- | | 7.60 (s, 1H), 7.50-7.41 (m, 2H), 7.37 (dt, J = |
| | oxononyl)amino)ethoxy)isoxazol- | | 8.0, 3.8 Hz, 4H), 7.21 (t, J = 7.8 Hz, 3H), 6.88 |
| | 5-yl)-3-methylbutanoyl)-4- | | (d, J = 7.9 Hz, 2H), 6.51 (s, 2H), 6.09 (d, J = 5.6 |
| | hydroxy-N-((S)-1-(4-(4- | | Hz, 1H), 5.12 (d, J = 3.8 Hz, 1H), 4.91 (t, J = 7.1 |
| | methylthiazol-5- | | Hz, 1H), 4.49-4.33 (m, 3H), 4.32-4.14 (m, |
| | yl)phenyl)ethyl)pyrrolidine-2- | | 5H), 4.00 (s, 1H), 3.76-3.63 (m, 2H), 3.56- |
| | carboxamide | | 3.44 (m, 2H), 3.12 (t, J = 6.8 Hz, 2H), 3.06- |
| | | | 2.97 (m, 1H), 2.70-2.62 (m, 1H), 2.46 (s, 3H), |
| | | | 2.30-2.21 (m, 1H), 2.12 (t, J = 7.4 Hz, 2H), |
| | | | 2.02 (d, J = 9.8 Hz, 1H), 1.84-1.73 (m, 1H), |
| | | | 1.56-1.43 (m, 4H), 1.38 (d, J = 7.0 Hz, 3H), |
| | | | 1.25 (s, 8H), 1.17-1.08 (m, 1H), 0.96 (d, J = |
| | | | 6.4 Hz, 3H), 0.80 (d, J = 6.7 Hz, 3H). 0.95 (s, |
| | | | 9H). |
| 212 | (2S,4R)-1-[(2R)-2-[3-(2-[[10- | 1044.4 | ¹H NMR (400 MHz, DMSO-d6) δ 14.38 (s, 1H), |
| | ([[4-(2-[[3-amino-6-(2- | | 8.99 (d, J = 2.3 Hz, 1H), 8.42 (d, J = 7.7 Hz, 1H), |
| | hydroxyphenyl)pyridazin-4- | | 8.26 (t, J = 6.0 Hz, 1H), 8.07-7.86 (m, 1H), |
| | yl]oxy]ethyl)phenyl]methyl] | | 7.59 (s, 1H), 7.52-7.30 (m, 6H), 7.29-7.08 |
| | carbamoyl)decyl]amino]ethoxy)- | | (m, 3H), 6.87 (t, J = 7.7 Hz, 2H), 6.51 (s, 2H), |
| | 1,2-oxazol-5-yl]-3-methylbutanoyl | | 6.09 (d, J = 1.4 Hz, 1H), 5.11 (d, J = 3.6 Hz, 1H), |
| | ]-4-hydroxy-N-[(1S)-1-[4-(4- | | 4.91 (q, J = 7.0 Hz, 1H), 4.39 (dt, J = 26.4, 7.4 |
| | methyl-1,3-thiazol-5- | | Hz, 3H), 4.22 (d, J = 6.0 Hz, 5H), 3.78-3.60 |
| | yl)phenyl]ethyl]pyrrolidine-2- | | (m, 1H), 3.62-3.37 (m, 2H), 3.12 (t, J = 6.9 Hz, |
| | carboxamide | | 3H), 2.95 (s, 2H), 2.71-2.58 (m, 2H), 2.45 (s, |
| | | | 3H), 2.29-2.16 (m, 1H), 2.11 (t, J = 7.4 Hz, 2H), |
| | | | 2.03 (t, J= 10.0 Hz, 1H), 1.78 (dd, J= 12.7, 8.2 |
| | | | Hz, 1H), 1.55-1.44 (m, 4H), 1.37 (d, J = 7.0 Hz, |
| | | | 3H), 1.24 (s, 12H), 0.96 (t, J = 6.5 Hz, 3H), 0.81 |
| | | | (dd, J= 15.0, 6.7 Hz, 3H). |
| 240 | (2S,4R)-1-((2R)-2-(3-(2- | 955.90 | ¹H NMR (400 MHz, DMSO-d₆) δ 14.20 (s, 1H), |
| | (6-(4-(2-((3-amino-6-(2- | | 6 10.96 (s, 1H),8.99 (d, J= 2.5 Hz, 1H), 8.40 (d, |
| | hydroxyphenyl)pyridazin-4- | | J = 7.7 Hz, 1H), 7.86 (d, J = 6.7 Hz, 1H), 7.68- |
| | yl)oxy)ethyl)benzoyl)-3,6- | | 7.60 (m, 3H),7.54-7.41 (m, 4H),7.40-7.34 (m, |
| | diazabicyclo[3.2.0]heptan-3- | | 2H), 733-7.22 (m, 1H), 6.90 (dd, J = 11.1,7.9 |
| | yl)ethoxy)isoxazol-5-yl)-3- | | Hz, 2H), 6.76 (s, 1H), 6.18 (s, 1H), 5.12-5.04 |
| | methylbutanoyl)-4-hydroxy-N- | | (m, 2H), 4.91 (p, J = 7.0 Hz, 1H), 4.59-4.48 (m, |
| | ((S)-1-(4-(4-methylthiazol-5- | | 4H), 4.36 (t, J= 7.9 Hz, 1H), 4.29 (s, 2H), 4.00- |
| | yl)phenyl)ethyl)pyrrolidine-2- | | 3.89 (m, 2H), 3.75-3.65 (m, 3H), 3.47 (s, 2H), |
| | carboxamide | | 3.21 (s, 2H), 2.45 (s, 3H), 2.21-2.19 (m, 1H) |
| | | | 2.02 (d, J = 9.4 Hz, 1H), 1.78 (ddd, J = 12.7, 8.1, |
| | | | 4.7 Hz, 1H), 1.48-1.35 (m, 3H), 1.24 (s, 1H), |
| | | | 0.97 (t, J = 6.1 Hz, 3H), 0.82 (dd, J = 14.4, 6.6 |
| | | | Hz, 3H). |
| 213 | (2S,4R)-1-((R)-2-(3-(2-(5- | 966.40 | ¹H NMR (400 MHz, DMSO-d6) δ 14.36 (s, 1H), |
| | (4-(2-((3-amino-6-(2- | | 8.99 (d, J = 2.2 Hz, 1H), 8.41 (d, J = 7.7 Hz, 1H), |
| | hydroxyphenyl)pyridazin-4- | | 8.13 (s, 0.4H, FA), 7.97-7.91 (m, 1H), 7.61 (s, |
| | yl)oxy)ethyl)benzoyl)-3,4,5,6- | | 1H), 7.55-7.46 (m, 4H), 7.46-7.33 (m, 4H), |
| | tetrahydropyrrolo[3,4-c]pyrrol- | | 7.27-7.19 (m, 1H), 6.91-6.83 (m, 2H), 6.53 |
| | 2(1H)-yl)ethoxy)isoxazol-5-yl)- | | (d, J = 3.0 Hz, 2H),6.11 (s, 1H),5.11 (d, J = 3.6 |
| | 3-methylbutanoyl)-4-hydroxy- | | Hz, 1H), 4.96-4.85 (m, 1H), 4.50 (t, J = 6.7 Hz, |
| | N-((S)-1-(4-(4-methylthiazol-5- | | 2H), 4.42-4.20 (m, 6H), 4.14 (s, 2H), 3.73- |
| | yl)phenyl)ethyl)pyrrolidine-2- | | 3.62 (m, 4H), 3.59-3.41 (m, 3H), 3.20 (t, J = |
| | carboxamide | | 6.7 Hz, 2H), 2.56-2.51 (m, 2H), 2.46 (s, 3H), |
| | | | 2.25-2.15 (m, 1H), 2.07-1.98 (m, 1H), 1.83- |
| | | | 1.73 (m, 1H), 1.37 (d, J = 7.0 Hz, 3H), 0.96 |
| | | | (d, J = 6.4 Hz, 3H), 0.82 (d, J = 6.4 Hz, 3H). |
| 222 | (2S,4R)-1-((R)-2-(3-(2-(1′- | 970.42 | ¹H NMR (400 MHz, DMSO-d6) δ 14.37 (s, 1H), |
| | (4-(2-((3-amino-6-(2- | | 8.99 (s, 1H), 8.42 (d, J = 7.7 Hz, 1H), 7.94 (d, |
| | hydroxyphenyl)pyridazin-4- | | J = 7.9 Hz, 1H), 7.63-7.55 (m, 3H), 7.46 (dd, J = |
| | yl)oxy)ethyl)benzoyl)-[3,3'- | | 18.8, 8.1 Hz, 4H), 7.36 (d, J = 7.8 Hz, 2H), 7.23 |
| | biazetidin]-1-yl)ethoxy)isoxazol- | | (t, J = 7.9 Hz, 1H), 6.88 (d, J = 7.9 Hz, 2H), 6.53 |
| | 5-yl)-3-methylbutanoyl)-4- | | (s, 2H), 6.07 (s, 1H), 5.11 (d, J = 3.6 Hz, 1H), |
| | hydroxy-N-((S)-1-(4-(4- | | 4.91 (q, J = 7.0 Hz, 1H), 4.49 (t, J = 6.9 Hz, 2H), |
| | methylthiazol-5-yl) | | 4.40-4.25 (m, 3H), 4.09 (d, J = 8.2 Hz, 3H), |
| | phenyl)ethyl)pyrrolidine-2- | | 3.95 (s, 1H), 3.75-3.61 (m, 3H), 3.59-3.41 |
| | carboxamide | | (m, 2H), 3.20 (t, J = 6.9 Hz, 2H), 2.93 (s, 0H), |
| | | | 3.05-2.82 (m, 2H), 2.75 (s, 2H), 2.67 (s, 2H), |
| | | | 2.45 (s, 3H), 2.26-2.18 (m, 2H), 2.03 (t, J = |
| | | | 10.0 Hz, 1H), 1.77 (ddd, J = 12.8, 8.0, 4.7 Hz, |
| | | | 1H), 1.41 (dd, J = 29.4, 7.0 Hz, 3H), 0.95 (d, J = |
| | | | 6.5 Hz, 3H), 0.80 (dd, J = 14.6, 6.7 Hz, 3H). |
| 225 | (2S,4R)-1-((2R)-2-(3-(2- | 956.12 | ¹H NMR (300 MHz, DMSO-d6) δ 14.43 (s,1H), |
| | (3-(4-(2-((3-amino-6-(2- | | 8.99 (s, 1H), 8.42 (d, J = 7.6 Hz, 1H), 8.14 (s, |
| | hydroxyphenyl)pyridazin-4- | | 1H), 7.95 (d, J = 8.3 Hz, 1H), 7.64-7.61 (m, |
| | yl)oxy)ethyl)benzoyl)-3,6- | | 1H), 7.47 (s, 5H), 7.43 (s, 1H), 7.35 (s, 2H), 7.24 |
| | diazabicyclo[3.2.0]heptan-6- | | (t, J = 7.7 Hz, 1H), 6.88 (d, J = 7.8 Hz, 2H), 6.54 |
| | yl)ethoxy)isoxazol-5-yl)-3- | | (s, 2H), 6.05 (d, J = 21.6 Hz, 1H), 5.11 (d, J = |
| | methylbutanoyl)-4-hydroxy-N- | | 3.5 Hz, 1H), 4.91 (t, J = 7.1 Hz, 1H), 4.51 (s, |
| | ((S)-1-(4-(4-methylthiazol-5- | | 2H), 4.38 (t, J = 8.0 Hz, 1H), 4.28 (s, 1H), 4.09 |
| | yl)phenyl)ethyl)pyrrolidine-2- | | (s, 3H), 4.01 (s, 2H), 3.67 (s, 4H), 3.59-3.45 |
| | carboxamide | | (m, 2H), 3.19 (d, J = 6.4 Hz, 4H), 2.96 (s, 3H), |
| | | | 2.46 (s, 3H), 2.34-1.93 (m, 1H), 1.77 (d, J = |
| | | | 8.1 Hz, 0H), 1.37 (d, J = 6.9 Hz, 3H), 1.24 (s, |
| | | | 0H), 0.96 (s, 3H), 0.79 (s, 3H). |
| 217 | (2S,4R)-1-((R)-2-(3-(2- | 796.40 | ¹H NMR (400 MHz, DMSO-d6) δ 14.24 (s, 1H), |
| | (4-(3-amino-6-(2- | | 8.99 (d, J = 1.1 Hz, 1H), 8.42 (d, J = 7.6 Hz, 1H), |
| | hydroxyphenyl)pyridazin-4- | | 7.95-7.87 (m, 1H), 7.50 (s, 1H), 7.49-7.40 |
| | yl)piperazin-1-yl) | | (m, 2H), 7.40-7.33 (m, 2H), 7.24 (tt, J = 7.2, |
| | ethoxy)isoxazol-5-yl)-3- | | 2.3 Hz, 1H), 6.93-6.83 (m, 2H), 6.26 (s, 2H), |
| | methylbutanoyl)-4-hydroxy-N- | | 6.12 (s, 1H), 5.11 (d, J = 3.6 Hz, 1H), 4.91 (p, |
| | ((S)-1-(4-(4-methylthiazol-5- | | J = 7.1, 6.5 Hz, 1H), 4.41-4.27 (m, 4H), 3.74- |
| | yl)phenyl)ethyl)pyrrolidine-2- | | 3.62 (m, 2H), 3.49-3.42 (m, 1H), 3.11 (s, 4H), |
| | carboxamide | | 2.78 (t, J = 5.5 Hz, 2H), 2.71 (s, 4H), 2.46 (s, |
| | | | 3H), 2.36-2.10 (m, 1H), 2.10-1.98 (m, 1H), |
| | | | 1.83-1.72 (m, 1H), 1.38 (d, J = 7.0 Hz, 3H), |
| | | | 1.00-0.93 (m, 3H), 0.82 (dd, J = 13.8, 6.6 Hz, |
| | | | 3H). |
| 229 | (2S,4R)-1-((R)-2-(3-(2-((7-((4- | 988.45 | ¹H NMR (400 MHz, DMSO-d6) δ 14.42-14.33 |
| | (2-((3-amino-6-(2- | | (m, 1H), 8.99 (d, J = 2.5 Hz, 1H), 8.41 (d, J = 7.9 |
| | hydroxyphenyl)pyridazin-4- | | Hz, 1H), 8.27 (t, J = 5.9 Hz, 1H), 7.94 (d, J = 7.7 |
| | yl)oxy)ethyl)benzyl)amino)-7- | | Hz, 1H), 7.59 (s, 1H), 7.49-7.41 (m, 2H), 7.40- |
| | oxoheptyl)amino)ethoxy)isoxazol- | | 7.32 (m, 4H), 7.27-7.17 (m, 3H), 6.90-6.84 |
| | 5-yl)-3-methylbutanoyl)-4- | | (m, 2H), 6.52-6.47 (m, 2H), 6.11 (s, 1H), 5.12 |
| | hydroxy-N-((S)-1-(4-(4- | | (d, J = 3.6 Hz, 1H), 4.95-4.87 (m, 1H), 4.43 (t, |
| | methylthiazol-5- | | J = 6.8 Hz, 2H), 4.36 (t, J = 7.8 Hz, 1H), 4.32- |
| | yl)phenyl)ethyl)pyrrolidine- | | 4.25 (m, 3H), 4.23 (d, J = 5.8 Hz, 2H), 3.73- |
| | 2-carboxamide | | 3.63 (m, 2H), 3.60-3.50 (m, 2H), 3.50-3.39 |
| | | | (m, 2H), 3.12 (t, J = 6.8 Hz, 2H), 2.79-2.69 (m, |
| | | | 1H), 2.45 (s, 3H), 2.30-2.15 (m, 2H), 2.11 (t, |
| | | | J = 7.4 Hz, 2H), 2.07-1.98 (m, 1H), 1.83-1.73 |
| | | | (m, 1H), 1.57-1.41 (m, 4H), 1.37 (d, J = 7.0 Hz, |
| | | | 3H), 1.32-1.25 (m, 4H), 0.96 (d, J = 6.5 Hz, |
| | | | 3H), 0.81 (d, J= 6.5 Hz, 3H). |
| 218 | (2S,4R)-1-((R)-2-(3-(2-((5- | 960.0 | ¹H NMR (400 MHz, DMSO-d6) δ 14.38 (s, 1H), |
| | ((4-(2-((3-amino-6-(2- | | 8.99 (d, J = 1.8 Hz, 1H), 8.41 (d, J = 7.7 Hz, 1H), |
| | hydroxyphenyl)pyridazin-4- | | 8.27 (s, 1H), 7.94 (d, J= 7.9 Hz, 1H), 7.59 (s, |
| | yl)oxy)ethyl)benzyl)amino)-5- | | 1H), 7.43 (d, J = 8.3 Hz, 2H), 7.39-7.33 (m, |
| | oxopentyl)amino)ethoxy)isoxaz | | 4H), 7.27-7.17 (m, 3H), 6.87 (t, J = 7.7 Hz, 2H), |
| | ol-5-yl)-3-methylbutanoyl)-4- | | 6.50 (s, 2H), 6.08 (s, 1H), 5.10 (d, J = 3.5 Hz, |
| | hydroxy-N-((S)-1-(4-(4- | | 1H), 4.91 (q, J = 7.3 Hz, 1H), 4.40 (dt, J = 27.1, |
| | methylthiazol-5-yl) | | 7.4 Hz, 3H), 4.30-4.16 (m, 5H), 3.86-3.54 (m, |
| | phenyl)ethyl)pyrrolidine-2- | | 2H), 3.57-3.38 (m, 1H), 3.12 (t, J = 6.7 Hz, 2H), |
| | carboxamide | | 2.88 (s, 2H), 2.69-2.64 (m, 2H), 2.45 (s, 3H), |
| | | | 2.33 (q, J = 1.9 Hz, 1H), 2.17-2.09 (m, 2H), |
| | | | 2.01 (d, J = 8.8 Hz, 1H), 1.82-1.74 (m, 1H), |
| | | | 1.53 (d, J = 7.7 Hz, 2H), 1.37 (d, J = 7.0 Hz, 2H), |
| | | | 1.24 (m, 3H), 0.98 (m, 1H), 0.95 (d, J = 6.5 Hz, |
| | | | 3H), 0.81 (dd, J= 15.2, 6.6 Hz, 3H). |
| 230 | (2S,4R)-1-((R)-2-(3-(2-((3- | 932.41 | 1H NMR (300 MHz, DMSO-d6) δ 8.99 (s, 1H), |
| | ((4-(2-((3-amino-6-(2- | | 8.43 (d, J = 7.3 Hz, 2H), 8.28 (s, 2H), 7.95 (d, |
| | hydroxyphenyl)pyridazin-4- | | J = 7.7 Hz, 1H), 7.60 (s, 1H), 7.50-7.40 (m, 2H), |
| | yl)oxy)ethyl)benzyl)amino)-3- | | 7.40-7.31 (m, 4H), 7.24 (m, J = 7.8, 1.8 Hz, |
| | oxopropyl)amino)ethoxy)isoxazol- | | 3H), 6.87 (m, J = 8.2, 6.8 Hz, 2H), 6.51 (s, 2H), |
| | 5-yl)-3-methylbutanoyl)-4- | | 6.07 (s, 1H), 4.91 (t, J = 7.1 Hz, 1H), 4.40 (m, |
| | hydroxy-N-((S)-1-(4-(4- | | J = 18.9, 7.4 Hz, 3H), 4.25 (d, J = 5.8 Hz, 2H), |
| | methylthiazol-5-yl) | | 4.18 (t, J = 5.5 Hz, 2H), 3.77-3.57 (m, 1H), |
| | phenyl)ethyl)pyrrolidine-2- | | 3.12 (t, J = 6.9 Hz, 2H), 2.83 (m, J = 26.1, 6.1 |
| | carboxamide | | Hz, 3H), 2.46 (s, 3H), 2.33-2.27 (m, 2H), 2.22 |
| | | | (d, J = 7.9 Hz, 1H), 2.03 (t, J = 10.2 Hz, 1H), |
| | | | 1.78 (m, J = 12.8, 8.0, 4.7 Hz, 1H), 1.41 (m, J = |
| | | | 22.1,7.0 Hz, 3H), 0.95 (d, J = 6.3 Hz, 3H), 0.81 |
| | | | (m, J = 11.4, 6.6 Hz, 3H). |
| 233 | (2S,4R)-1-((2S)-2-(2-(4-(2-((4- | 973.50 | ¹H NMR (400 MHz, Methanol-d⁴) δ 8.83 (s, 1H), |
| | (3-(3-amino-6-(2-hydroxyphenyl) | | 7.79-7.70 (m, 2H), 7.48-7.42 (m, 3H), 7.42- |
| | pyridazin-4-yl)-3,8- | | 7.37 (m, 2H), 7.25-7.19 (m, 1H), 6.91-6.84 |
| | diazabicyclo[3.2.1] | | (m, 2H), 6.53 (dd, J= 6.2, 2.1 Hz, 1H), 6.18 (d, |
| | octan-8-yl)pyridin-2- | | J = 2.1 Hz, 1H), 4.61 (s, 1H), 4.58-4.51 (m, |
| | yl)oxy)ethyl)piperazin-1- | | 2H), 4.51-4.45 (m, 3H), 4.37-4.30 (m, 3H), |
| | yl)acetamido)-3,3- | | 3.90-3.84 (m, 1H), 3.82-3.76 (m, 1H), 3.36- |
| | dimethylbutanoyl)-4-hydroxy- | | 3.32 (m, 2H), 3.15-2.99 (m, 4H), 2.84-2.77 |
| | N-(4-(4-methylthiazol-5- | | (m, 2H), 2.76-2.55 (m, 8H), 2.46 (d, J = 9.6 Hz, |
| | yl)benzyl)pyrrolidine-2- | | 3H), 2.26-2.17 (m, 3H), 2.17-2.02 (m, 3H), |
| | carboxamide | | 1.03 (s, 9H). |
| 243 | (2S,4R)-1-((2S)-2-(2-(4- | 1014.50 | ¹H NMR (400 MHz, DMSO-d⁶, with a drop of |
| | ((1r,3S)-3-((4-(3-(3-amino-6-(2- | | D₂O) 58.97 (s, 1H), 8.61 (t, J = 6.2 Hz, 1H),8.19 |
| | hydroxyphenyl)pyridazin-4-yl)- | | (s, 3H, FA), 7.91 (d, J = 7.9 Hz, 1H), 7.84-7.74 |
| | 3,8-diazabicyclo[3.2.1]octan-8- | | (m, 1H), 7.49 (s, 1H), 7.44-7.37 (m, 4H), 7.23 |
| | yl)pyridin-2-yl)oxy) | | (t, J = 7.7 Hz, 1H), 6.91-6.81 (m, 2H), 6.55- |
| | cyclobutoxy)piperidin-1- | | 6.50 (m, 1H), 6.15-6.11 (m, 1H), 5.97-5.91 |
| | yl)acetamido)-3,3- | | (m, 1H), 5.22-5.11 (m, 1H), 4.52-4.32 (m, |
| | dimethylbutanoyl)-4-hydroxy- | | 6H), 4.32-4.22 (m, 2H), 3.69-3.56 (m, 4H), |
| | N-(4-(4-methylthiazol-5- | | 3.28-3.17 (m, 2H), 3.05-2.97 (m, 3H), 2.94- |
| | yl)benzyl)pyrrolidine-2- | | 2.86 (m, 1H), 2.75-2.64 (m, 2H), 2.44 (s, 3H), |
| | carboxamide, tri-formic acid | | 2.36-2.12 (m, 8H), 2.09-1.75 (m, 6H), 1.54- |
| | | | 1.37 (m, 2H), 0.94 (s, 9H). |
| 244 | (2S,4R)-1-((S)-2-(2-(4-((1-(3- | 1045.53 | ¹H NMR (300 MHz, DMSO-d6) δ 14.38 (s, 1H), |
| | ((4-(2-((3-amino-6-(2- | | 8.98 (s, 1H), 8.60 (s, 1H), 8.50(s,1H) 7.94 (d, J = |
| | hydroxyphenyl)pyridazin-4- | | 7.9 Hz, 1H), 7.80 (s, 1H), 7.59 (s, 1H), 7.38 (d, |
| | yl)oxy)ethyl)benzyl)amino)-3- | | J = 18.5 Hz, 6H), 7.23 (d, J = 7.9 Hz, 3H), 6.88 |
| | oxopropyl)piperidin-4- | | (d, J = 7.8 Hz, 2H), 6.52 (d, J = 9.3 Hz, 2H), 5.15 |
| | yl)oxy)piperidin-1- | | (d, J = 3.4 Hz, 1H), 4.54-4.33 (m, 6H), 4.33- |
| | yl)acetamido)-3,3- | | 4.18 (m, 3H), 3.74-3.52 (m, 2H), 3.34 (s, 3H), |
| | dimethylbutanoyl)-4-hydroxy- | | 3.13 (t, J = 6.7 Hz, 2H), 2.95 (d, J = 24.1 Hz, |
| | N-(4-(4-methylthiazol-5- | | 4H), 2.78-2.61 (m, 2H), 2.45 (s, 3H), 2.33- |
| | yl)benzyl)pyrrolidine-2- | | 2.17 (m, 1H), 2.14-2.00(m,5H), 1.68 (s, 6H), |
| | carboxamide | | 1.45 (s, 5H), 0.94 (s, 9H). |
| 237 | (2S,4R)-1-((R)-2-(3-(2-(3-((1- | 1014.15 | ¹H NMR (300 MHz, DMSO-d6) δ 14.30 ( br s, |
| | (4-(2-((3-amino-6-(2- | | 1H), 9.00 (s, 1H), 8.41 (d, J = 7.7 Hz, 1H), 8.14 |
| | hydroxyphenyl)pyridazin-4- | | (s, 0.4H, FA), 7.98-7.89 (m, 1H), 7.61 (s, 1H), |
| | yl)oxy)ethyl)benzoyl)piperidin- | | 7.52-7.41 (m, 4H), 7.40-7.30 (m, 4H), 7.24 |
| | 4-yl)oxy)azetidin-1- | | (td, J = 7.6, 1.5 Hz, 1H), 6.93-6.83 (m, 2H), |
| | yl)ethoxy)isoxazol-5-yl)-3- | | 6.54 (s, 2H), 6.12 (s, 1H), 5.12 (d, J = 3.5 Hz, |
| | methylbutanoyl)-4-hydroxy-N- | | 1H), 4.91 (t, J = 7.2 Hz, 1H), 4.50 (t, J = 6.8 Hz, |
| | ((S)-1-(4-(4-methylthiazol-5- | | 2H), 4.42-4.18 (m, 5H), 4.02 (s, 3H), 3.75- |
| | yl)phenyl)ethyl)pyrrolidine-2- | | 3.57 (m, 5H), 3.54-3.40 (m, 2H), 3.19 (t, J = |
| | carboxamide | | 6.8 Hz, 5H), 2.46 (s, 3H), 2.33-2.15 (m, 1H), |
| | | | 2.11-1.96 (m, 1H), 1.87-1.69 (m, 3H), 1.54- |
| | | | 1.29 (m, 5H), 0.97 (dd, J = 6.6, 4.1 Hz, 3H), 0.82 |
| | | | (dd, J = 10.8, 6.6 Hz, 3H). |
| 253 | (2S,4R)-1-((R)-2-(3-(2-(4-((1- | 508.00 | IH NMR (300 MHz, DMSO-d6) δ 14.38 (s, 1H), |
| | (4-(2-((3-amino-6-(2- | (M+2)²⁺ | 8.99 (s, 1H), 8.42 (d, J = 7.7 Hz, 1H), 7.98-7.88 |
| | hydroxyphenyl)pyridazin-4- | | (m, 1H), 7.65-7.56 (m, 3H), 7.52-7.41 (m, 4H), |
| | yl)oxy)ethyl)benzoyl)azetidin-3- | | 7.41-7.34 (m, 2H), 7.24 (td, J = 7.6, 1.5 Hz, 1H), |
| | yl)oxy)piperidin-1- | | 6.96-6.81 (m, 2H), 6.53 (s, 2H), 6.10 (s, 1H), |
| | yl)ethoxy)isoxazol-5-yl)-3- | | 5.11 (d, J = 3.6 Hz, 1H), 4.97-4.85 (m, 1H),4.57- |
| | methylbutanoyl)-4-hydroxy-N- | | 4.06 (m, 10H), 3.90-3.40 (m, 4H), 3.21 (t, J = |
| | ((S)-1-(4-(4-methylthiazol-5- | | 6.8 Hz, 2H), 2.85-2.70 (m, 2H), 2.65 (1, J = 5.6 |
| | yl)phenyl)ethyl)pyrrolidine-2- | | Hz, 2H), 2.46 (s, 3H), 2.33-1.95 (m, 5H), 1.86- |
| | carboxamide | | 1.68 (m, 3H), 1.53-1.28 (m, 5H), 0.97 (d, J = 6.6, |
| | | | 4.5 Hz, 3H), 0.80 (d, J = 6.6 Hz, 3H). |
| 263 | (2S,4R)-1-((S)-2-(11-(4-(4-(2- | 1016.57 | ¹H NMR (300 MHz, DMSO-d6) δ 14.39 (s, 1H), |
| | ((3-amino-6-(2- | | 8.99 (s, 1H), 8.38 (d, J = 7.8 Hz, 1H), 7.99-7.90 |
| | hydroxyphenyl)pyridazin-4- | | (m, 1H), 7.79 (d, J = 9.2 Hz, 1H), 7.60 (s, 1H), |
| | yl)oxy)ethyl)benzyl)piperazin-1- | | 7.52-7.27 (m, 7H), 7.23 (m, J = 7.8, 2.7 Hz, |
| | yl)undecanamido)-3,3- | | 3H), 6.87 (m, J = 8.1,6.8 Hz, 2H), 6.52 (s, 2H), |
| | dimethylbutanoyl)-4-hydroxy-N- | | 5.10 (d, J = 3.5 Hz, 1H), 4.97-4.87 (m, 1H), |
| | ((S)-1-(4-(4-methylthiazol-5- | | 4.57-4.36 (m, 4H), 4.28 (s, 1H), 3.60 (s, 2H), |
| | yl)phenyl)ethyl)pyrrolidine-2- | | 3.41 (s, 2H), 3.13 (t, J = 6.8 Hz, 2H), 2.46 (s, |
| | carboxamide | | 2H), 2.32 (s, 5H), 2.21 (t, J = 6.9 Hz, 3H), 2.15- |
| | | | 1.95 (m, 3H), 1.87-1.74 (m, 1H), 1.47 (d, J = |
| | | | 7.2 Hz, 2H), 1.38 (d, J = 6.9 Hz, 5H), 1.24 (s, |
| | | | 14H), 0.94 (s, 9H). |
| 264 | (2S,4R)-1-((2S)-2-(11-(5-(4-(2- | 1042.59 | ¹H NMR (300 MHz, DMSO-d6,drop of D₂O) δ |
| | ((3-amino-6-(2- | | 8.96 (s, 1H), 8.39 (d, J = 7.8 Hz, 1H), 8.28 (s, |
| | hydroxyphenyl)pyridazin-4- | | 2H), 7.94-7.85 (m, 1H), 7.79 (d, J = 9.2 Hz, |
| | yl)oxy)ethyl)benzyl)hexahydrop | | 1H), 7.54 (s, 1H), 7.47-7.29 (m, 6H), 7.24 (d, |
| | yrrolo[3,4-c]pyrrol-2(1H)- | | J = 7.9 Hz, 3H), 6.93-6.83 (m, 2H), 4.89 (t, J = |
| | yl)undecanamido)-3,3- | | 7.2 Hz, 1H), 4.54-4.35 (m, 4H), 4.27 (s, 1H), |
| | dimethylbutanoyl)-4-hydroxy-N- | | 3.57-3.54 (m, 1H), 3.51 (s, 2H), 3.12 (t, J = 6.7 |
| | ((S)-1-(4-(4-methylthiazol-5- | | Hz, 2H), 2.74 (p, J = 1.9 Hz, 1H), 2.65-2.58 |
| | yl)phenyl)ethyl)pyrrolidine-2- | | (m, 2H), 2.53 (m, 7H), 2.44-2.31 (s, 1H), 2.22 |
| | carboxamide | | (t, J = 7.2 Hz, 1H), 2.07 (m, J = 19.2, 9.8 Hz, |
| | | | 2H), 1.86-1.75 (m, 1H), 1.44 (s, 4H), 1.36 (d, |
| | | | J = 6.9 Hz, 3H), 1.23 (s, 12H), 0.92 (s, 9H). |
| 251 | (2S,4R)-1-((R)-2-(3-(2-((4-(2- | 875.30 | ¹H NMR (300 MHz, DMSO-d6) δ 14.39 (s, 1H), |
| | ((3-amino-6-(2- | | 8.99 (s, 1H), 8.43 (d, J = 7.6 Hz, 1H), 7.95 (d, |
| | hydroxyphenyl)pyridazin-4- | | J = 7.8 Hz, 1H), 7.61 (s, 1H), 7.50-7.40 (m, 2H), |
| | yl)oxy)ethyl)benzyl)(methyl) | | 7.40-7.31 (m, 4H), 7.30-7.18 (m, 3H), 6.87 (dd, |
| | amino)ethoxy)isoxazol-5-yl)-3- | | J = 8.0, 6.6 Hz, 2H), 6.51 (s, 2H), 6.10 (s, 1H), |
| | methylbutanoyl)-4-hydroxy-N- | | 5.11 (d, J = 3.6 Hz, 1H), 4.93 (m, 1H), 4.46 |
| | ((S)-1-(4-(4-methylthiazol-5- | | (m,2H), 4.38 (t, J = 7.9 Hz, 1H), 4.27 (d, J = 5.8 |
| | yl)phenyl)ethyl)pyrrolidine-2- | | Hz, 3H), 3.68 (dd, J = 15.9, 8.0 Hz, 2H), 3.53 (s, |
| | carboxamide | | 2H), 3.49-3.41 (m, 1H), 3.14 (t, J = 6.9 Hz, 2H), |
| | | | 2.73 (s, 2H), 2.31-2.10 (m, 4H), 2.03 (t, J = 10.3 |
| | | | Hz, 1H), 1.78 (ddd, J = 12.9, 8.1, 4.9 Hz, 1H), |
| | | | 1.38 (d, J = 7.0 Hz, 3H), 1.32-1.14 (m, 3), 0.96 |
| | | | (d, J = 6.4 Hz, 3H), 0.88-0.63 (m, 3H). |

### Example 10. Preparation of 5-(4-(2-((4-(2-((3-amino-6-(2-hydroxyphenyl)pyridazin-4-yl)oxy)ethyl)benzyl)amino)ethyl)piperazin-1-yl)-2-(2,6-dioxopiperidin-3-yl)isoindoline-1,3-dione (compound 3)

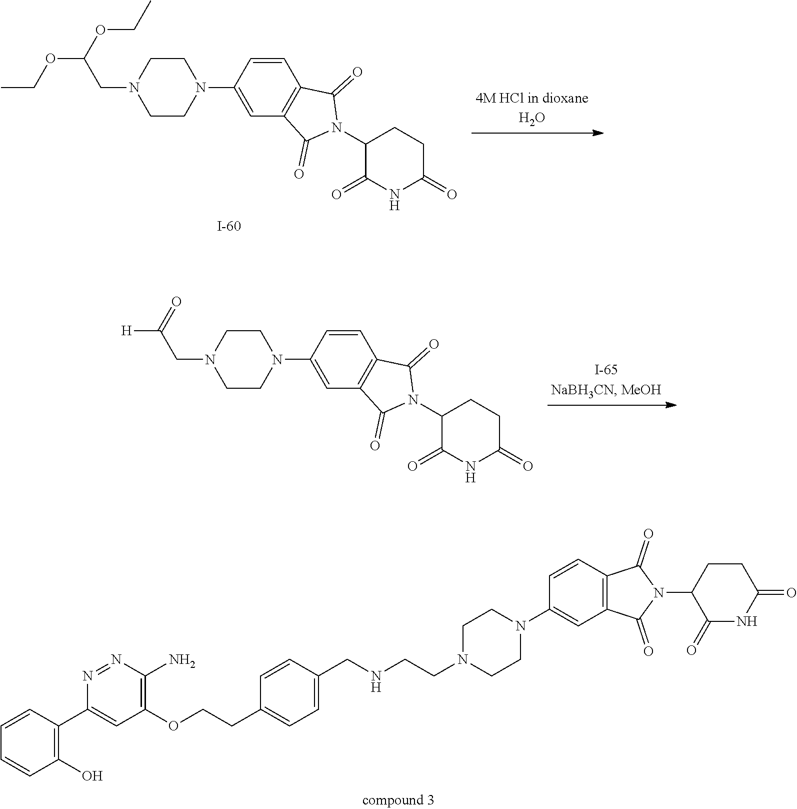

### Step 1: Preparation of 2-(4-(2-(2,6-dioxopiperidin-3-yl)-1,3-dioxoisoindolin-5-yl)piperazin-1-yl)acetaldehyde

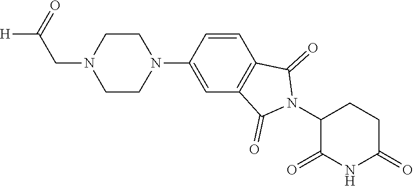 [SMILES: O=CCN1CCN(c2ccc3c(c2)C(=O)N(C2CCC(=O)NC2=O)C3=O)CC1]

To a stirred solution of 5-[4-(2,2-diethoxyethyl)piperazin-1-yl]-2-(2,6-dioxopiperidin-3-yl)isoindole-1,3-dione (I-60, 60.00 mg, 0.131 mmol, 1.00 equiv) in 4 M HCl in dioxane (1.50 mL) was added water (1.50 mL). The resulting mixture was stirred for 16 h at 50° C. The mixture was basified to pH 8 with saturated aqueous NaHCO₃. The resulting mixture was extracted with EtOAc (3×10 mL). The combined organic layers were washed with brine (20 mL) and dried over anhydrous Na₂SO₄. After filtration, the filtrate was concentrated under reduced pressure. This resulted in 2-(4-(2-(2,6-dioxopiperidin-3-yl)-1,3-dioxoisoindolin-5-yl)piperazin-1-yl)acetaldehyde (50 mg, 96.22%) as a yellow solid that was used in the next step without further purification. LCMS (ESI) m/z: [M+H]⁺=385.

### Step 2: Preparation of 5-(4-(2-((4-(2-((3-amino-6-(2-hydroxyphenyl)pyridazin-4-yl)oxy)ethyl)benzyl)amino)ethyl)piperazin-1-yl)-2-(2,6-dioxopiperidin-3-yl)isoindoline-1,3-dione (compound 3)

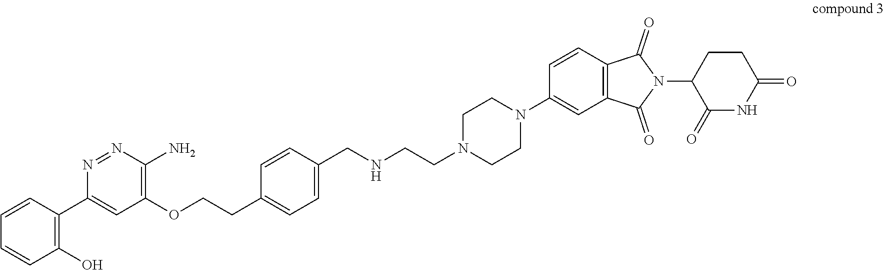 [SMILES: Nc1nnc(-c2ccccc2O)cc1OCCc1ccc(CNCCN2CCN(c3ccc4c(c3)C(=O)N(C3CCC(=O)NC3=O)C4=O)CC2)cc1]

To a stirred mixture of 2-[4-[2-(2,6-dioxopiperidin-3-yl)-1,3-dioxoisoindol-5-yl]piperazin-1-yl]acetaldehyde (2, 50.00 mg, 0.130 mmol, 1.00 equiv) and 2-(6-amino-5-[2-[4-(aminomethyl)phenyl]ethoxy]pyridazin-3-yl)phenol (I-65, 43.76 mg, 0.130 mmol, 1.00 equiv) in MeOH (1.00 mL) was added NaBH₃CN (32.70 mg, 0.520 mmol, 4.00 equiv) in portions at room temperature. The resulting mixture was stirred for 2 h then concentrated under reduced pressure. The crude product was purified by reversed-phase preparative HPLC to afford compound 3 (2.5 mg, 2.67%) as a yellow solid. ¹H NMR (400 MHz, Methanol-d4) δ 8.57 (s, 2H), 7.78-7.70 (m, 1H), 7.66 (d, J=8.5 Hz, 1H), 7.51-7.31 (m, 6H), 7.29-6.86 (m, 4H), 5.13-5.03 (m, 1H), 4.62 (s, 2H), 4.56 (t, J=6.3 Hz, 2H), 3.98 (s, 2H), 3.42 (t, J=5.1 Hz, 4H), 3.26 (t, J=6.3 Hz, 2H), 2.99-2.65 (m, 6H), 2.57 (dd, J=13.8, 5.9 Hz, 6H), 2.17-2.07 (m, 1H). LCMS (ESI) m/z: [M+H]⁺=705.45.

### Example 11. Preparation of 1-(4-(2-((3-amino-6-(2-hydroxyphenyl)pyridazin-4-yl)oxy)ethyl)benzyl)-3-(3-((2-(2,6-dioxopiperidin-3-yl)-1,3-dioxoisoindolin-4-yl)oxy)propyl)urea (compound 8)

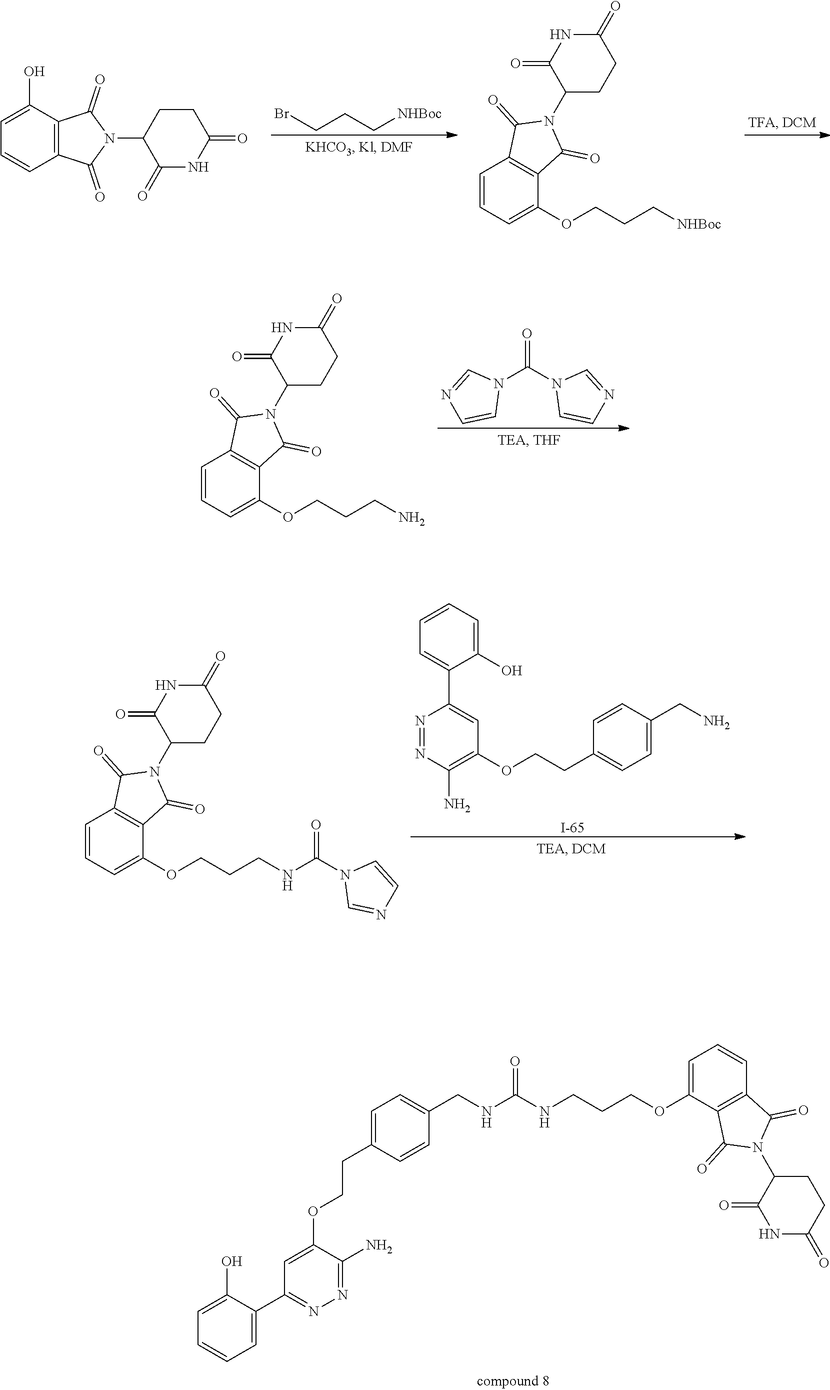

### Step 1: Preparation of tert-butyl (3-((2-(2,6-dioxopiperidin-3-yl)-1,3-dioxoisoindolin-4-yl)oxy)propyl)carbamate

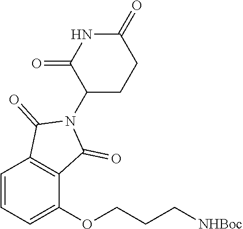 [SMILES: CC(C)(C)OC(=O)NCCCOc1cccc2c1C(=O)N(C1CCC(=O)NC1=O)C2=O]

To a stirred mixture of 2-(2,6-dioxopiperidin-3-yl)-4-hydroxyisoindole-1,3-dione (100.00 mg, 0.365 mmol, 1.00 equiv) and tert-butyl N-(3-bromopropyl)carbamate (95.52 mg, 0.401 mmol, 1.10 equiv) in DMF (3.0 mL) were added KI (6.05 mg, 0.036 mmol, 0.10 equiv) and KHCO₃ (73.01 mg, 0.729 mmol, 2.00 equiv) in portions at room temperature under an atmosphere of dry nitrogen. The resulting mixture was stirred overnight at 65° C. then diluted with EtOAc (20 mL) and washed with brine (2×20 mL). The organic layer was dried over Na₂SO₄ and filtered. The filtrate was concentrated and the residue purified by reversed phase flash chromatography to afford tert-butyl (3-((2-(2,6-dioxopiperidin-3-yl)-1,3-dioxoisoindolin-4-yl)oxy)propyl)carbamate (94 mg, 58.55%) as a yellow solid. LCMS (ESI) m/z [M+H]⁺=432.

### Step 2: Preparation of 4-(3-aminopropoxy)-2-(2,6-dioxopiperidin-3-yl)isoindoline-1,3-dione

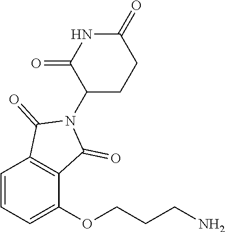 [SMILES: NCCCOc1cccc2c1C(=O)N(C1CCC(=O)NC1=O)C2=O]

To a solution of tert-butyl (3-((2-(2,6-dioxopiperidin-3-yl)-1,3-dioxoisoindolin-4-yl)oxy)propyl)carbamate (94.00 mg, 0.218 mmol, 1.00 equiv) in DCM (2.0 mL) was added TFA (1.00 mL) dropwise at room temperature. The resulting mixture was stirred for 1 h then concentrated under reduced pressure. The residue was purified by reversed phase flash chromatography to afford 4-(3-aminopropoxy)-2-(2,6-dioxopiperidin-3-yl)isoindoline-1,3-dione (40 mg, 62.34%) as a brown oil. LCMS (ESI) m/z [M+H]⁺=332.

### Step 3: Preparation of N-(3-((2-(2,6-dioxopiperidin-3-yl)-1,3-dioxoisoindolin-4-yl)oxy)propyl)-1H-imidazole-1-carboxamide

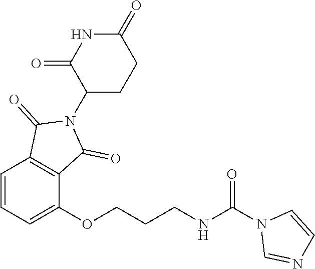 [SMILES: O=C1CCC(N2C(=O)c3cccc(OCCCNC(=O)n4ccnc4)c3C2=O)C(=O)N1]

A solution of 4-(3-aminopropoxy)-2-(2,6-dioxopiperidin-3-yl)isoindoline-1,3-dione (40.00 mg, 0.121 mmol, 1.00 equiv) in THF (1.0 mL) was treated with carbonyldiimidazole (39.15 mg, 0.241 mmol, 2.00 equiv) at 0° C. under an atmosphere of dry nitrogen followed by addition of TEA (12.22 mg, 0.121 mmol, 1.00 equiv). The resulting mixture was stirred for 4 h at 0° C. and then concentrated under reduced pressure to afford crude N-(3-((2-(2,6-dioxopiperidin-3-yl)-1,3-dioxoisoindolin-4-yl)oxy)propyl)-1H-imidazole-1-carboxamide (80 mg) as a white solid. This product was used in the next step without further purification. LCMS (ESI) m/z [M+H]⁺=426.

### Step 4: Preparation of 1-(4-(2-((3-amino-6-(2-hydroxyphenyl)pyridazin-4-yl)oxy)ethyl)benzyl)-3-(3-((2-(2,6-dioxopiperidin-3-yl)-1,3-dioxoisoindolin-4-yl)oxy)propyl)urea (compound 8)

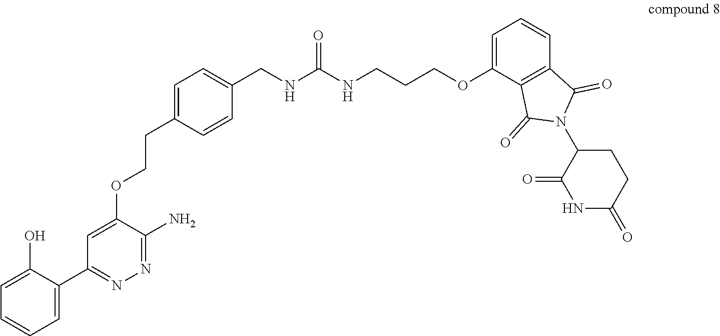 [SMILES: Nc1nnc(-c2ccccc2O)cc1OCCc1ccc(CNC(=O)NCCCOc2cccc3c2C(=O)N(C2CCC(=O)NC2=O)C3=O)cc1]

To a stirred mixture of N-(3-((2-(2,6-dioxopiperidin-3-yl)-1,3-dioxoisoindolin-4-yl)oxy)propyl)-1H-imidazole-1-carboxamide (80.00 mg, 0.188 mmol, 1.00 equiv, crude) and 2-(6-amino-5-[2-[4-(aminomethyl)phenyl]ethoxy]pyridazin-3-yl)phenol (I-65, 40 mg, 0.188 mmol, 1.00 equiv) in DCM (4.0 mL) was added TEA (40 mg, 0.564 mmol, 3.0 equiv) dropwise at room temperature under an atmosphere of dry nitrogen. The resulting mixture was stirred for 3 h at 50° C. and then concentrated under reduced pressure. The crude product was purified by reversed-phase preparative HPLC to afford compound 8 (20.4 mg) as an off-white solid. ¹H NMR (300 MHz, DMSO-d₆) δ 11.13 (s, 1H), 7.82 (t, J=7.9 Hz, 1H), 7.73 (d, J=7.8 Hz, 1H), 7.65 (s, 1H), 7.49 (dd, J=13.6, 7.9 Hz, 2H), 7.33 (d, J=7.8 Hz, 4H), 7.20 (d, J=7.7 Hz, 3H), 6.96 (q, J=7.7 Hz, 2H), 6.20 (d, J=39.4 Hz, 2H), 5.09 (dd, J=12.9, 5.4 Hz, 1H), 4.50 (t, J=6.8 Hz, 2H), 4.31-4.09 (m, 4H), 3.25-3.17 (m, 5H), 2.94-2.73 (m, 2H), 2.65-2.59 (m, 1H), 2.09-1.81 (m, 3H). LCMS (ESI) m/z [M+H]⁺=694.20.

### Example 12. Preparation of (2S,4R)-1-[(2S)-2-(10-[[4-(2-[[3-amino-6-(2-hydroxyphenyl)pyridazin-4-yl](methyl)amino]ethyl)phenyl]formamido]decanamido)-3,3-dimethylbutanoyl]-4-hydroxy-N-[[4-(4-methyl-1,3-thiazol-5-yl)phenyl]methyl]pyrrolidine-2-carboxamide (compound 20)

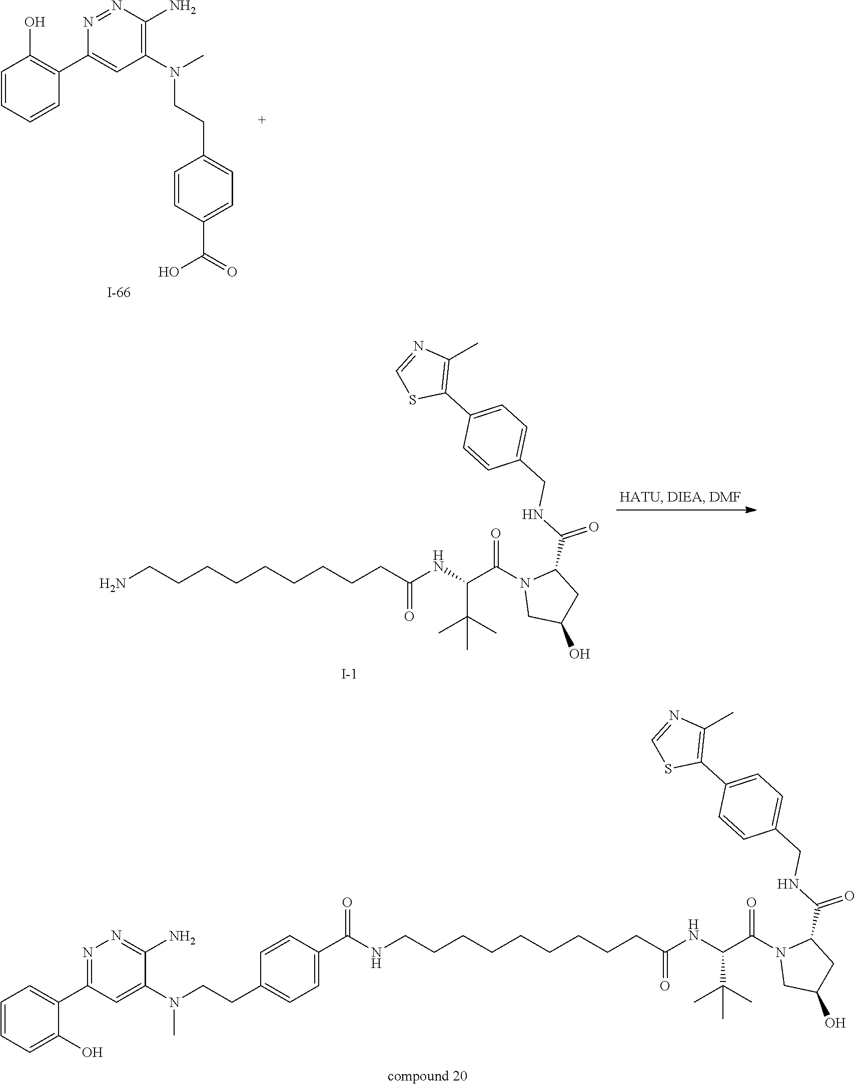

To a stirred solution of (2S,4R)-1-((S)-2-(10-aminodecanamido)-3,3-dimethylbutanoyl)-4-hydroxy-N-(4-(4-methylthiazol-5-yl)benzyl)pyrrolidine-2-carboxamide (I-1, 48 mg, 0.08 mmol, 1.2 equiv) in DMF (1.0 mL) was added HATU (30 mg, 0.080 mmol, 1.20 equiv). The resulting mixture was stirred for 30 min at room temperature followed by addition of 4-(2-[[3-amino-6-(2-hydroxyphenyl)pyridazin-4-yl](methyl)amino]ethyl)benzoic acid (I-66, 40 mg, 0.07 mmol, 1.0 equiv) and DIEA (26 mg, 0.2 mmol, 3.0 equiv). The resulting mixture was stirred for an additional 2 h at room temperature. Then purified directly by reversed-phase preparative HPLC to afford compound 20 (3.6 mg, 5.71%) as a white solid. ¹H NMR (400 MHz, DMSO-d6) δ 14.31 (s, 1H), 8.98 (s, 1H), 8.58 (t, J=6.1 Hz, 1H), 8.34 (t, J=5.7 Hz, 1H), 7.91-7.84 (m, 2H), 7.76-7.74 (m, 2H), 7.47 (s, 1H), 7.43-7.37 (m, 4H), 7.32 (d, J=8.2 Hz, 2H), 7.26-7.24 (m, 1H), 6.91-6.87 (m, 2H), 6.12 (s, 2H), 5.14 (d, J=3.5 Hz, 1H), 4.54 (d, J=9.3 Hz, 1H), 4.47-4.39 (m, 2H), 4.35 (s, 1H), 4.21 (m, 1H), 3.66 (s, 2H), 3.22-3.20 (m, 2H), 2.94 (s, 3H), 2.86 (t, J=7.8 Hz, 2H), 2.44 (s, 3H), 2.29-2.22 (m, 1H), 2.14-2.09 (m, 1H), 2.06-2.00 (m, 1H), 1.98-1.87 (m, 1H), 1.49-1.47 (m, 4H), 1.27-1.24 (m, 12H), 0.93 (s, 9H). LCMS (ESI) m/z: [M+H]⁺=946.7

The following compounds in Table D5 were prepared using procedures similar to those used for the preparation of compound 20.

TABLE D5

| | | LCMS | |
|---|---|---|---|
| | | (ESI) | |
| No. | Name | m/z | ¹H NMR |
| 134 | (2S,4R)-1-[(2S)-2-(10-[[4-(2-[[3- | 932.5 | ¹H NMR (400 MHz, DMSO-d6) δ 14.03 (s, 1H), |
| | amino-6-(2- | | 10.52 (s, 1H), 8.98 (s, 1H), 8.56 (t, J = 6.0 Hz, 1H), |
| | hydroxyphenyl)pyridazin-4- | | 8.37 (t, J = 5.6 Hz, 1H), 7.98 (s, 1H), 7.83 (d, J = |
| | yl]amino]ethyl)phe- | | 9.3 Hz, 1H), 7.82-7.67 (m, 2H), 7.47-7.41 (m, |
| | nyl]formamido]decanamido)-3,3- | | 3H), 7.40-7.36 (m, 4H), 7.16 (s, 2H), 7.04 (d, J = |
| | dimethylbutanoyl]-4-hydroxy-N- | | 8.2 Hz, 1H), 7.01-6.84 (m, 2H), 5.12 (s, 1H), 4.54 |
| | [[4-(4-methyl-1,3-thiazol-5- | | (d, J = 9.4 Hz, 1H), 4.49-4.32 (m, 3H), 4.31- |
| | yl)phenyl]methyl]pyrrolidine-2- | | 4.07 (m, 1H), 3.75-3.64 (m, 4H), 3.27-3.20 (m, |
| | carboxamide | | 2H), 3.02 (t, J = 7.3 Hz, 2H), 2.44 (s, 3H), 2.32- |
| | | | 2.19 (m, 1H), 2.18-1.99 (m, 2H), 1.96-1.84 (m, |
| | | | 1H), 1.49 (s, 4H), 1.33-1.19 (m, 10H), 0.93 (s, |
| | | | 9H). |
| 144 | (2S,4R)-1-((S)-2-(10-(4-(2-((3- | 960.6 | ¹H NMR (400 MHz, DMSO-d6) δ 14.26 (s, 1H), |
| | amino-6-(2- | | 8.98 (s, 1H), 8.56 (t, J = 6.0 Hz, 1H), 8.32 (t, J = |
| | hydroxyphenyl)pyridazin-4- | | 5.7 Hz, 1H), 7.94-7.81 (m, 2H), 7.74 (d, J = 7.9 |
| | yl)(ethyl)amino)eth- | | Hz, 2H), 7.58 (s, 1H), 7.40 (q, J = 8.1 Hz, 4H), 7.32- |
| | yl)benzamido)decanamido)-3,3- | | 7.19 (m, 3H), 6.93-6.85 (m, 2H), 6.12 (s, 2H), |
| | dimethylbutanoyl)-4-hydroxy-N- | | 5.12 (d, J = 3.5 Hz, 1H), 4.54 (d, J = 9.3 Hz, 1H), |
| | (4-(4-methylthiazol-5- | | 4.48-4.38 (m, 2H), 4.37-4.32 (m, 1H), 4.26- |
| | yl)benzyl)pyrrolidine-2- | | 4.16 (m, 1H), 3.68-3.60 (m, 2H), 3.44 (t, J = 7.7 |
| | carboxamide | | Hz, 2H), 3.38-3.35 (m, 2H), 3.24-3.16 (m, 2H), |
| | | | 2.78 (t, J = 7.7 Hz, 2H), 2.44 (s, 3H), 2.31-2.19 |
| | | | (m, 1H), 2.16-1.97 (m, 2H), 1.92-1.87 (m, 1H), |
| | | | 1.50-1.46 (m, 4H), 1.27-1.22 (m, 10H), 1.04 (t, |
| | | | J = 7.0 Hz, 3H), 0.93 (s, 9H). |
| 242 | (2S,4R)-1-((2S)-2-(4-(4-(2-((4- | 1001.55 | ¹H NMR (300 MHz, DMSO-d6) δ 14.14 (s, 1H), |
| | (3-(3-amino-6-(2- | | 8.98 (s, 1H), 8.57 (t, J = 6.1 Hz, 1H), 7.95-7.82 |
| | hydroxyphenyl)pyridazin-4-yl)- | | (m, 2H), 7.79 (d, J = 6.0 Hz, 1H), 7.50 (s, 1H), 7.47- |
| | 3,8-diazabicyclo[3.2.1]octan-8- | | 7.34 (m, 4H), 7.28-7.17 (m, 1H), 6.91-6.79 |
| | yl)pyridin-2- | | (m, 2H), 6.54 (dd, J = 6.1, 2.1 Hz, 1H), 6.16 (d, J = |
| | yl)oxy)ethyl)piperazin-1- | | 2.1 Hz, 1H), 5.98 (s, 2H), 5.13 (s, 1H), 4.59-4.32 |
| | yl)butanamido)-3,3- | | (m, 6H), 4.32-4.15 (m, 3H), 3.66 (d, J = 4.5 Hz, |
| | dimethylbutanoyl)-4-hydroxy-N- | | 2H), 3.28-3.22 (m, 2H), 3.01 (d, J = 11.6 Hz, 2H), |
| | (4-(4-methylthiazol-5- | | 2.62 (t, J = 5.9 Hz, 2H), 2.45 (s, 4H), 2.40-2.33 |
| | yl)benzyl)pyrrolidine-2- | | (m, 5H), 2.30-1.79 (m, 12H), 1.69-1.58 (m, 2H), |
| | carboxamide | | 0.94 (s, 9H). |
| 234 | (2S,4R)-1-((2S)-2-(5-(4-(2-((4- | 1015.0 | ¹H NMR (300 MHz, DMSO-d6) δ 14.15 (s, 1H), |
| | (3-(3-amino-6-(2- | | 8.99 (s, 1H), 8.57 (t, J = 6.0 Hz, 1H), 7.96-7.83 |
| | hydroxyphenyl)pyridazin-4-yl)- | | (m, 2H), 7.79 (d, J = 6.0 Hz, 1H), 7.50 (s, 1H), 7.47- |
| | 3,8-diazabicyclo[3.2.1]octan-8- | | 7.34 (m, 4H), 7.23 (ddd, J = 8.5, 7.1, 1.5 Hz, 1H), |
| | yl)pyridin-2- | | 6.92-6.79 (m, 2H), 6.54 (dd, J = 6.0, 2.0 Hz, 1H), |
| | yl)oxy)ethyl)piperazin-1- | | 6.16 (d, J = 2.0 Hz, 1H), 5.97 (d, J = 5.1 Hz, 2H), |
| | yl)pentanamido)-3,3- | | 5.13 (s, 1H), 4.75-4.10 (m, 9H), 3.66 (d, J = 4.6 |
| | dimethylbutanoyl)-4-hydroxy-N- | | Hz, 2H), 3.31 (s, 2H), 3.25 (d, J = 11.5 Hz, 2H), |
| | (4-(4-methylthiazol-5- | | 3.01 (d, J = 11.6 Hz, 2H), 2.63 (s, 2H), 2.45 (s, 5H), |
| | yl)benzyl)pyrrolidine-2- | | 2.39-2.03 (m, 10H), 2.00-1.93 (m, 4H), 1.44 (d, |
| | carboxamide | | J = 24.1 Hz, 4H), 0.94 (s, 9H). |
| 260 | (2S,4R)-1-((S)-2-(5-(4-(3-((4-(2- | 989.40 | ¹H NMR (400 MHz, DMSO-d₆) δ 14.37 (s, 1H), 8.98 |
| | ((3-amino-6-(2- | | (s, 1H), 8.56 (t, J = 6.1 Hz, 1H), 8.39 (t, J = 5.9 Hz, |
| | hydroxyphenyl)pyridazin-4- | | 1H), 7.97-7.90 (m, 1H), 7.84 (d, J = 9.3 Hz, 1H), |
| | yl)oxy)ethyl)benzyl)amino)-3- | | 7.59 (s, 1H), 7.42-7.31 (m, 6H), 7.27-7.19 (m, |
| | oxopropyl)piperazin-1- | | 3H), 6.87 (t, J = 7.6 Hz, 2H), 6.49 (s, 2H), 5.13 (d, |
| | yl)pentanamido)-3,3- | | J = 3.5 Hz, 1H), 4.54 (d, J = 9.3 Hz, 1H), 4.43 (td, J = |
| | dimethylbutanoyl)-4-hydroxy-N- | | 7.1, 4.0 Hz, 4H), 4.35 (s, 1H), 4.22 (dd, J = 18.4, |
| | (4-(4-methylthiazol-5- | | 5.5 Hz, 3H), 3.66-3.61 (m, 2H), 3.14-3.09 (m, |
| | yl)benzyl)pyrrolidine-2- | | 2H), 2.44 (s, 3H), 2.38-1.90 (m, 15H), 1.52-1.34 |
| | carboxamide | | (m, 4H), 0.93 (s, 9H). |
| 254 | (2S,4R)-1-((S)-2-(15-(4-(2-((3- | 1017.35 | ¹H NMR (300 MHz, DMSO-d6) δ 14.37 (br s, 1H), |
| | amino-6-(2- | | 8.99 (s, 1H), 8.38 (d, J = 7.4 Hz, 2H), 8.00-7.89 (m, |
| | hydroxyphenyl)pyridazin-4- | | 1H), 7.80 (dd, J = 8.8, 5.1 Hz, 3H), 7.61 (s, 1H), 7.52- |
| | yl)oxy)ethyl)benzamido)penta- | | 7.32 (m, 6H), 7.24 (td, J = 7.6, 1.5 Hz, 1H), 6.94- |
| | decanamido)-3,3- | | 6.83 (m, 2H), 6.52 (s, 2H), 5.10 (d, J = 3.5 Hz, 1H), |
| | dimethylbutanoyl)-4-hydroxy-N- | | 4.98-4.86 (m, 1H), 4.57-4.36 (m, 4H), 4.28 (s, |
| | ((S)-1-(4-(4-methylthiazol-5- | | 1H), 3.60 (s, 2H), 3.27-3.15 (m, 4H), 2.46 (s, 3H), |
| | yl)phenyl)ethyl)pyrrolidine-2- | | 2.32-2.19 (m, 1H), 2.16-1.92 (m, 2H), 1.87-1.73 |
| | carboxamide | | (m, 1H), 1.58-1.43 (m, 4H), 1.38 (d, J = 7.0 Hz, |
| | | | 3H), 1.25 (s, 20H), 0.94 (s, 9H). |
| 246 | N1-(4-(2-((3-amino-6-(2- | 1003.30 | ¹H NMR (400 MHz, DMSO-d6) δ 14.39 (s, 1H), |
| | hydroxyphenyl)pyridazin-4- | | 8.98 (s, 1H), 8.37 (d, J = 7.8 Hz, 1H), 8.26 (t, J = |
| | yl)oxy)ethyl)benzyl)-N15-((S)-1- | | 6.0 Hz, 1H), 7.98-7.88 (m, 2H), 7.59 (s, 1H), 7.46- |
| | ((2S,4R)-4-hydroxy-2-(((S)-1- | | 7.40 (m, 2H), 7.40-7.31 (m, 4H), 7.27-7.16 |
| | (4-(4-methylthiazol-5- | | (m, 3H), 6.86 (t, J = 8.0 Hz, 2H), 6.51 (s, 2H), 5.08 |
| | yl)phenyl)ethyl)carbamoyl)pyrrolidin- | | (d, J = 3.6 Hz, 1H), 4.90 (p, J = 7.0 Hz, 1H), 4.47- |
| | 1-yl)-3-methyl-1-oxobutan- | | 4.37 (m, 3H), 4.33 (t, J = 8.3 Hz, 1H), 4.28 (q, J = |
| | 2-yl)pentadecanediamide | | 3.7 Hz, 1H), 4.22 (d, J = 5.9 Hz, 2H), 3.60 (d, J = |
| | | | 3.3 Hz, 2H), 3.12 (t, J = 6.9 Hz, 2H), 2.45 (s, 3H), |
| | | | 2.23-2.07 (m, 4H), 2.06-1.87 (m, 2H), 1.83- |
| | | | 1.72 (m, 1H), 1.52-1.42 (m, 4H), 1.37 (d, J = 7.0 |
| | | | Hz, 3H), 1.22 (s, 18H), 0.85 (dd, J = 19.4, 6.6 Hz, |
| | | | 6H). |
| 262 | (2S,4R)-1-((S)-2-(5-(4-(3-((4-(2- | 1003.50 | ¹H NMR (400 MHz, DMSO-d₆) δ 14.38 (s, 1H), 8.98 |
| | ((3-amino-6-(2- | | (s, 1H), 8.42-8.34 (m, 2H), 7.97-7.91 (m, 1H), |
| | hydroxyphenyl)pyridazin-4- | | 7.78 (d, J = 9.2 Hz, 1H), 7.59 (s, 1H), 7.49-7.41 |
| | yl)oxy)ethyl)benzyl)amino)-3- | | (m, 2H), 7.40-7.31 (m, 4H), 7.27-7.19 (m, 3H), |
| | oxopropyl)piperazin-1- | | 6.87 (t, J = 7.9 Hz, 2H), 6.49 (s, 2H), 5.10 (d, J = |
| | yl)pentanamido)-3,3- | | 3.5 Hz, 1H), 4.90 (q, J = 7.3 Hz, 1H), 4.51 (d, J = |
| | dimethylbutanoyl)-4-hydroxy-N- | | 9.3 Hz, 1H), 4.42 (q, J = 7.7, 7.1 Hz, 3H), 4.30- |
| | ((S)-1-(4-(4-methylthiazol-5- | | 4.21 (m, 3H), 3.60 (d, J = 4.4 Hz, 2H), 3.12 (t, J = |
| | yl)phenyl)ethyl)pyrrolidine-2- | | 6.8 Hz, 2H), 2.45 (s, 3H), 2.43-1.98 (m, 15H), 1.79 |
| | carboxamide | | (ddd, J = 12.8, 8.4, 4.6 Hz, 1H), 1.50-1.42 (m, |
| | | | 2H), 1.37-1.30 (m, 5H), 0.93 (s, 9H). |

### Example 13. Preparation of N-(2-[4-[3-amino-6-(2-hydroxyphenyl)pyridazin-4-yl]piperazin-1-yl]ethyl)-9-[[2-(2,6-dioxopiperidin-3-yl)-1,3-dioxoisoindol-4-yl]amino]nonanamide (compound 97)

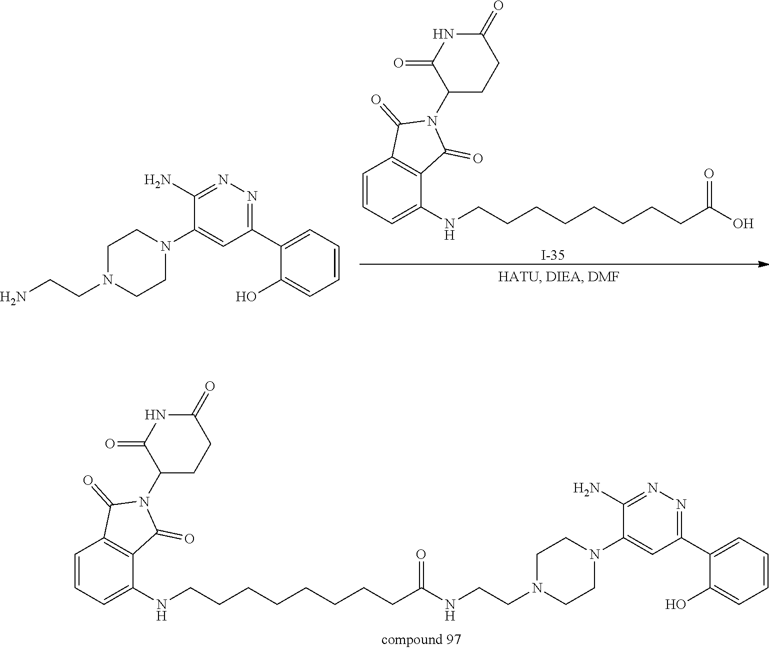

To a solution of 9-[[2-(2,6-dioxopiperidin-3-yl)-1,3-dioxoisoindol-4-yl]amino]nonanoic acid (I-35, 110.00 mg, 0.256 mmol, 1.00 equiv) in DMF (2.00 mL) were added HATU (126.60 mg, 0.333 mmol, 1.30 equiv), 2-[6-amino-5-[4-(2-aminoethyl)piperazin-1-yl]pyridazin-3-yl]phenol (I-67, 96.63 mg, 0.307 mmol, 1.20 equiv), and DIEA (99.31 mg, 0.768 mmol, 3.00 equiv). The mixture was stirred at 25° C. for 1 h. The resulting mixture was then purified by preparative HPLC to yield compound 97 (59.4 mg, 31.95%) as a yellow solid. ¹H NMR (300 MHz, DMSO-d6) δ 14.54-13.60 (m, 1H), 11.11 (s, 1H), 7.92 (dd, J=8.3, 1.7 Hz, 1H), 7.72 (t, J=5.7 Hz, 1H), 7.57 (dd, J=8.6, 7.0 Hz, 1H), 7.49 (s, 1H), 7.24 (td, J=7.6, 1.5 Hz, 1H), 7.18-6.98 (m, 2H), 6.98-6.80 (m, 2H), 6.51 (t, J=6.0 Hz, 1H), 6.25 (s, 2H), 5.05 (dd, J=12.8, 5.4 Hz, 1H), 3.33-3.20 (m, 1H), 3.10 (s, 3H), 3.00-2.82 (m, 1H), 2.72-2.57 (m, 5H), 2.50-2.40 (m, 2H), 2.06 (t, J=7.4 Hz, 3H), 1.66-1.37 (m, 4H), 1.39-1.14 (m, 9H). LCMS (ESI) m/z: [M+H]⁺=726.30

The following compounds in Table E1 were prepared using procedures similar to those used for the preparation of compound 97.

TABLE E1

| | | LCMS | |
|---|---|---|---|
| | | (ESI) | |
| No. | Name | m/z | ¹H NMR |
| 84 | N-(2-(4-(3-amino-6-(2- | 657.40 | ¹H NMR (400 MHz, DMSO-d6) δ 11.10 (s, 1H), |
| | hydroxyphenyl)pyridazin-4- | | 8.14 (s, 0.37H), 7.91 (dd, J = 8.4, 1.7 Hz, 1H), |
| | yl)piperazin-1-yl)ethyl)-4-((2- | | 7.87-7.73 (m, 2H), 7.60-7.34 (m, 3H), 7.25 |
| | (2,6-dioxopiperidin-3-yl)-1,3- | | (td, J = 7.6, 1.6 Hz, 1H), 6.90 (ddd, J = 7.0, 3.7, |
| | dioxoisoindolin-4- | | 2.4 Hz, 2H), 6.23 (s, 2H), 5.08 (dd, J = 12.7, |
| | yl)oxy)butanamide | | 5.4 Hz, 1H), 4.24 (t, J = 6.4 Hz, 2H), 3.24 (t, |
| | | | J = 6.4 Hz, 2H), 3.10 (s, 4H), 2.89 (ddd, J = 16.9, |
| | | | 13.8, 5.3 Hz, 1H), 2.73-2.59 (m, 4H), 2.56 (d, |
| | | | J = 11.4 Hz, 1H), 2.53 (m, 1H), 2.49 (s, 2H), |
| | | | 2.32 (t, J = 7.3 Hz, 2H), 2.02-1.94 (m, 3H) |
| 85 | N-(2-[4-[3-amino-6-(2- | 699.40 | ¹H NMR (400 MHz, Methanol-d4) δ 7.76 (dd, |
| | hydroxyphenyl)pyridazin-4- | | J = 8.5, 7.3 Hz, 1H), 7.71-7.62 (m, 2H), 7.43 |
| | yl]piperazin-1-yl]ethyl)-7-[[2- | | (dd, J = 8.0, 4.7 Hz, 3H), 7.04 (t, J = 7.7 Hz, |
| | (2,6-dioxopiperidin-3-yl)-1,3- | | 2H), 5.10 (dd, J = 12.7, 5.5 Hz, 1H), 4.24 (t, J = |
| | dioxoisoindol-4- | | 6.1 Hz, 2H), 3.89-3.40 (m, 10H), 2.96-2.64 |
| | yl]oxy]heptanamide | | (m, 3H), 2.32 (t, J = 7.4 Hz, 2H), 2.17-2.11 |
| | | | (m, J = 13.0, 5.5, 2.6 Hz, 1H), 1.91-1.85 (m, |
| | | | 2H), 1.76-1.68 (m, 2H), 1.65-1.53 (m, 2H), |
| | | | 1.48-1.42 (m, 2H), 1.42-1.36 (m, 2H) |
| 88 | N-(2-[4-[3-amino-6-(2- | 754.75 | ¹H NMR (300 MHz, Methanol-d4) δ 8.33 (s, |
| | hydroxyphenyl)pyridazin-4- | | 0.33H, FA), 7.79 (dd, J = 8.3, 1.6 Hz, 1H), 7.59- |
| | yl]piperazin-1-yl]ethyl)-11-[[2- | | 7.47 (m, 2H), 7.27 (ddd, J = 8.6, 7.3, 1.6 Hz, |
| | (2,6-dioxopiperidin-3-yl)-1,3- | | 1H), 7.07-6.89 (m, 4H), 5.07 (dd, J = 12.3, |
| | dioxoisoindol-4- | | 5.4 Hz, 1H), 4.62 (s, 3H), 4.61 (s, 2H), 3.54- |
| | yl]amino]undecanamide | | 3.13 (m, 5H), 2.93-2.53 (m, 8H), 2.24 (t, J = |
| | | | 7.3 Hz, 2H), 2.22 (t, J = 7.3 Hz, 1H), 1.63 (s, |
| | | | 4H), 1.34 (s, 11H) |
| 89 | N-(2-[4-[3-amino-6-(2- | 671.50 | ¹H NMR (300 MHz, Methanol-d4) δ 8.38 (s, |
| | hydroxyphenyl)pyridazin-4- | | 0.33H, FA), 7.75-7.60 (m, 2H), 7.44-7.32 |
| | yl]piperazin-1-yl]ethyl)-5-[[2- | | (m, 3H), 7.27 (td, J = 7.6, 1.5 Hz, 1H), 6.99- |
| | (2,6-dioxopiperidin-3-yl)-1,3- | | 6.86 (m, 2H), 5.08 (dd, J = 12.4, 5.5 Hz, 1H), |
| | dioxoisoindol-4- | | 4.63 (s, 1H), 4.26 (t, J = 5.7 Hz, 1H), 3.44 (t, |
| | yl]oxy]pentanamide(7.7 mg, 17.37%)4-[4- | | J = 6.3 Hz, 2H), 3.27-3.07 (m, 3H), 2.95-2.70 |
| | [(dimethylamino)methyl]-3,5- | | (m, 9H), 2.41 (t, J = 6.6 Hz, 2H), 2.21-2.04 |
| | dimethoxyphenyl]-2-methyl-1,2- | | (m, 1H), 1.98-1.89 (m, 4H) |
| | dihydro-2,7-naphthyridin-1-one | | |
| 90 | N-(2-[4-[3-amino-6-(2- | 713.45 | ¹H NMR (300 MHz, Methanol-d4) δ 7.82-7.66 |
| | hydroxyphenyl)pyridazin-4- | | (m, 2H), 7.52 (s, 1H), 7.37 (dd, J = 18.7, 7.9 |
| | yl]piperazin-1-yl]ethyl)-8-[[2- | | Hz, 2H), 7.26 (t, J = 7.7 Hz, 1H), 6.93 (d, J = |
| | (2,6-dioxopiperidin-3-yl)-1,3- | | 7.8 Hz, 2H), 5.09 (dd, J = 12.3, 5.5 Hz, 1H), |
| | dioxo-2,3-dihydro-1H-isoindol- | | 4.18 (t, J = 6.3 Hz, 2H), 3.42 (t, J = 6.4 Hz, |
| | 4-yl]oxy]octanamide | | 2H), 3.24 (s, 4H), 2.81 (d, J = 15.4 Hz, 6H), |
| | | | 2.69 (q, J = 8.5, 7.0 Hz, 3H), 2.25 (t, J = 7.2 |
| | | | Hz, 2H), 2.14 (d, J = 8.2 Hz, 1H), 1.91-1.77 |
| | | | (m, 2H), 1.76-1.53 (m, 4H), 1.44 (s, 4H) |
| 91 | N-(2-[4-[3-amino-6-(2- | 730.40 | ¹H NMR (300 MHz, Methanol-d4) δ 8.21 (s, |
| | hydroxyphenyl)pyridazin-4- | | 0.4H), 7.77 (d, J = 8.0 Hz, 1H), 7.61-7.48 (m, |
| | yl]piperazin-1-yl]ethyl)-3-[2-(2- | | 2H), 7.29 (t, J = 7.7 Hz, 1H), 7.05 (d, J = 2.0 |
| | [[2-(2,6-dioxopiperidin-3-yl)-1,3- | | Hz, 1H), 6.96-6.89 (m, 2H), 6.92-6.87 (m, 1H), |
| | dioxo-2,3-dihydro-1H-isoindol-5- | | 5.02 (dd, J = 12.4, 5.4 Hz, 1H), 3.78 (t, J = 5.9 |
| | yl]amino]ethoxy)ethoxy]propanamide | | Hz, 2H), 3.78-3.62 (m, 2H), 3.67 (s, 4H), 3.50- |
| | | | 3.43 (m, 4H), 3.41-3.30 (m, 4H), 3.01-2.90 (m, |
| | | | 4H), 2.87-2.60 (m, 4H), 2.59-2.51 (m, 1H), |
| | | | 2.50 (t, J = 5.9 Hz, 2H), 2.07-2.00 (m, 1H) |
| 92 | N-(2-[4-[3-amino-6-(2- | 726.40 | ¹H NMR (300 MHz, Methanol-d4) δ 8.44 (s, |
| | hydroxyphenyl)pyridazin-4- | | 0.22H, FA), 7.80 (dd, J = 8.3, 1.6 Hz, 1H), 7.61- |
| | yl]piperazin-1-yl]ethyl)-11-[[2- | | 7.47 (m, 2H), 7.27 (ddd, J = 8.6, 7.3, 1.6 Hz, |
| | (2,6-dioxopiperidin-3-yl)-1,3- | | 1H), 7.03-6.89 (m, 3H), 6.81 (dd, J = 8.4, 2.2 |
| | dioxoisoindol-4- | | Hz, 1H), 5.04 (dd, J = 12.3, 5.4 Hz, 1H), 4.61 |
| | yl]amino]undecanamide | | (s, 2H), 3.41 (t, J = 6.6 Hz, 2H), 3.19 (dd, J = |
| | | | 13.3, 6.3 Hz, 6H), 2.95-2.67 (m, 7H), 2.63 (t, |
| | | | J = 6.6 Hz, 2H), 2.23 (t, J = 7.3 Hz, 1H), 1.72- |
| | | | 1.60 (m, 4H), 1.50-1.32 (m, 8H) |
| 93 | N-(2-[4-[3-amino-6-(2- | 730.35 | ¹H NMR (300 MHz, Methanol-d4) δ 8.37 (s, |
| | hydroxyphenyl)pyridazin-4- | | 0.36 H, FA), 7.79 (d, J = 7.9 Hz, 1H), 7.50 (d, |
| | yl]piperazin-1-yl]ethyl)-11-[[2- | | J = 6.5 Hz, 2H), 7.26 (d, J = 8.2 Hz, 1H), 7.03 (d, |
| | (2,6-dioxopiperidin-3-yl)-1,3- | | J = 7.6 Hz, 2H), 6.94 (d, J = 7.9 Hz, 2H), 5.06 |
| | dioxoisoindol-5- | | (d, J = 12.9 Hz, 1H), 3.81-3.69 (m, 9H), 3.55- |
| | yl]amino]undecanamide | | 3.49 (m, 3H), 3.49-3.40 (m, 3H), 3.20 (s, |
| | | | 4H), 2.89-2.57 (m, 8H), 2.50 (d, J = 7.1 Hz, |
| | | | 1H), 2.13 (s, 1H) |
| 98 | N-(2-[4-[3-amino-6-(2- | 740.45 | ¹H NMR (300 MHz, Methanol-d4) δ 7.75 (d, J = |
| | hydroxyphenyl)pyridazin-4- | | 7.8 Hz, 1H), 7.61 (s, 1H), 7.55 (t, J = 7.8 Hz, |
| | yl]piperazin-1-yl]ethyl)-10-[[2- | | 1H), 7.35 (t, J = 7.6 Hz, 1H), 7.06-6.96 (m, |
| | (2,6-dioxopiperidin-3-yl)-1,3- | | 4H), 5.07 (dd, J = 12.3, 5.4 Hz, 1H), 3.56- |
| | dioxo-2,3-dihydro-1H-isoindol- | | 3.46 (m, 6H), 3.35-3.25 (m, 6H), 3.12-3.06 |
| | 4-yl]amino]decanamide | | (m, 2H), 2.97-2.68 (m, 3H), 2.26 (t, J = 7.4 |
| | | | Hz, 2H), 2.14 (s, 1H), 1.70-1.62 (m, 4H), 1.42- |
| | | | 1.30 (m, 10H) |
| 99 | N-(2-[4-[3-amino-6-(2- | 754.50 | ¹H NMR (300 MHz, Methanol-d4) δ 7.77 (dd, |
| | hydroxyphenyl)pyridazin-4- | | J = 8.3, 1.7 Hz, 1H), 7.65-7.51 (m, 2H), 7.31 |
| | yl]piperazin-1-yl]ethyl)-11-[[2- | | (ddd, J = 8.6, 7.3, 1.6 Hz, 1H), 7.02-6.91 (m, |
| | (2,6-dioxopiperidin-3-yl)-1,3- | | 3H), 6.82 (dd, J = 8.4, 2.2 Hz, 1H), 5.11-5.01 |
| | dioxo-2,3-dihydro-1H-isoindol- | | (m, 1H), 3.49 (t, J = 6.3 Hz, 2H), 3.36 (s, 3H), |
| | 5-yl]amino]undecanamide | | 3.19 (t, J = 7.0 Hz, 2H), 3.09 (d, J = 5.8 Hz, |
| | | | 4H), 2.90 (d, J = 6.4 Hz, 2H), 2.90-2.61 (m, |
| | | | 3H), 2.25 (t, J = 7.4 Hz, 2H), 2.17-2.11 (m, |
| | | | 1H), 1.64 (t, J = 6.8 Hz, 4H), 1.50-1.31 (m, |
| | | | 13H) |

### Example 14. Preparation of N-(4-(2-((3-amino-6-(2-hydroxyphenyl)pyridazin-4-yl)oxy)ethyl)benzyl)-5-((2-(2,6-dioxopiperidin-3-yl)-1,3-dioxoisoindolin-4-yl)oxy)-N-methylpentanamide (compound 10)

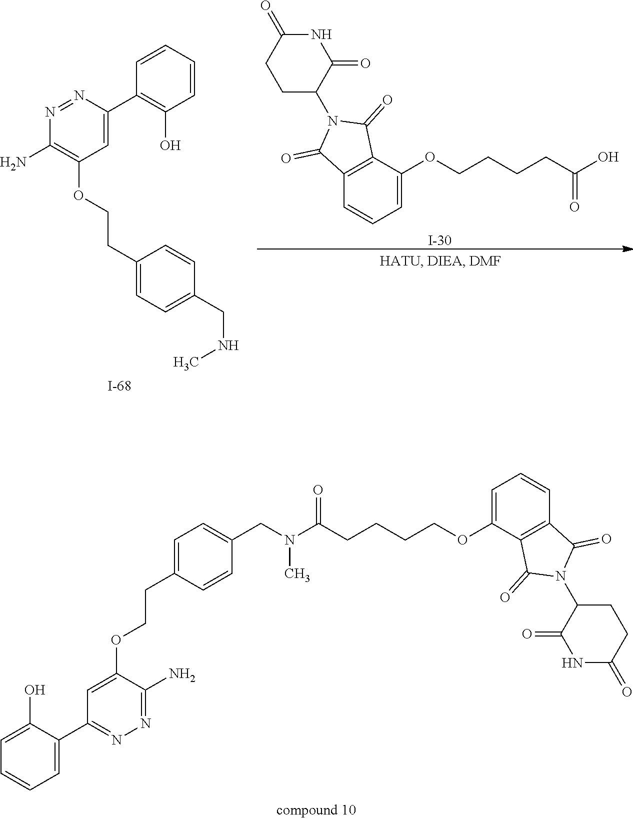

To a stirred mixture of I-68 (25.00 mg, 0.071 mmol, 1.00 equiv) and 5-[[2-(2,6-dioxopiperidin-3-yl)-1,3-dioxoisoindol-4-yl]oxy]pentanoic acid (I-30, 26.71 mg, 0.071 mmol, 1.00 equiv) in DMF (1.0 mL) were added DIEA (46.10 mg, 0.357 mmol, 5.00 equiv) and HATU (32.55 mg, 0.086 mmol, 1.2 equiv) at room temperature under an atmosphere of dry nitrogen. The resulting mixture was stirred for 1 h, then purified by reversed phase flash chromatography to afford compound 10 (16.5 mg, 32.87%) as a white solid. ¹H NMR (300 MHz, DMSO-d₆) δ 14.05 (s, 1H), 11.12 (s, 1H), 7.96-7.74 (m, 2H), 7.61 (s, 1H), 7.57-7.32 (m, 4H), 7.27 (t, J=7.7 Hz, 1H), 7.15 (t, J=8.3 Hz, 2H), 6.89 (t, J=8.6 Hz, 2H), 6.74 (s, 2H), 5.08 (dd, J=12.8, 5.4 Hz, 1H), 4.69-4.41 (m, 4H), 4.36-4.11 (m, 2H), 3.13 (t, J=6.9 Hz, 2H), 2.84 (d, J=33.9 Hz, 4H), 2.61 (d, J=2.9 Hz, 1H), 2.45 (d, J=8.9 Hz, 3H), 2.09-2.01 (m, 1H), 1.89-1.65 (m, 4H). LCMS (ESI) m/z [M+H]⁺=707.25.

The following compounds in Table E2 were prepared using procedures similar to those used for the preparation of compound 10.

TABLE E2

| | | LCMS | |
|---|---|---|---|
| | | (ESI) | |
| No. | Name | m/z | ¹H NMR |
| 146 | N-(4-(2-((3-amino-6-(2- | 706.2 | ¹H NMR (400 MHz, DMSO-d6) δ 14.33 (s, 1H), |
| | hydroxyphenyl)pyridazin-4- | | 11.10 (s, 1H), 8.34-8.17 (m, 1H), 7.89 (dd, |
| | yl)(methyl)amino)ethyl)benzyl)- | | J = 8.3, 1.7 Hz, 1H), 7.80 (dd, J = 8.6, 7.3 Hz, |
| | 5-((2-(2,6-dioxopiperidin-3-yl)- | | 1H), 7.50 (d, J = 8.5 Hz, 1H), 7.44 (t, J = 3.6 |
| | 1,3-dioxoisoindolin-4- | | Hz, 2H), 7.23 (td, J = 7.6, 7.1, 1.6 Hz, 1H), |
| | yl)oxy)pentanamide | | 7.20-7.11 (m, 4H), 6.93-6.82 (m, 2H), 6.09 |
| | | | (s, 2H), 5.08 (dd, J = 12.8, 5.4 Hz, 1H), 4.31- |
| | | | 4.09 (m, 4H), 3.38 (d, J = 6.3 Hz, 2H), 2.93 (s, |
| | | | 3H), 2.90-2.83 (m, 1H), 2.81-2.74 (m, 2H), |
| | | | 2.64-2.53 (m, 2H), 2.21 (t, J = 6.9 Hz, 2H), |
| | | | 2.02 (ddd, J = 11.9, 6.3, 3.8 Hz, 1H), 1.81- |
| | | | 1.64 (m, 4H). |
| 147 | N-(4-(2-((3-amino-6-(2- | 692.2 | ¹H NMR (400 MHz, DMSO-d6) δ 11.10 (s, 1H), |
| | hydroxyphenyl)pyridazin-4- | | 8.36 (s, 1H), 8.30 (t, J = 5.9 Hz, 1H), 7.88 (dd, |
| | yl)amino)ethyl)benzyl)-5-((2- | | J = 8.3, 1.7 Hz, 1H), 7.85-7.77 (m, 1H), 7.51 |
| | (2,6-dioxopiperidin-3-yl)-1,3- | | (d, J = 8.6 Hz, 1H), 7.44 (d, J = 7.3 Hz, 1H), |
| | dioxoisoindolin-4- | | 7.27 (d, J = 7.9 Hz, 2H), 7.19 (dd, J = 7.8, 6.0 |
| | yl)oxy)pentanamide | | Hz, 3H), 6.99 (s, 1H), 6.88-6.78 (m, 2H), 6.33 |
| | | | (s, 1H), 6.28 (s, 2H), 5.07 (dd, J = 12.8, 5.4 Hz, |
| | | | 1H), 4.31-4.16 (m, 4H), 3.49 (d, J = 10.0 Hz, |
| | | | 2H), 2.93 (t, J = 7.5 Hz, 2H), 2.90-2.77 (m, |
| | | | 1H), 2.57 (dd, J = 18.9, 4.6 Hz, 2H), 2.22 (t, J = |
| | | | 6.9 Hz, 2H), 2.07-1.97 (m, 1H), 1.74 (d, J = |
| | | | 5.5 Hz, 4H). |

### Example 15. Preparation of N-[(2-[4-[3-amino-6-(2-hydroxyphenyl)pyridazin-4-yl]piperazine-1-carbonyl]cyclopropyl)methyl]-2-[[2-(2,6-dioxopiperidin-3-yl)-1,3-dioxoisoindol-5-yl]oxy]acetamide (compound 78)

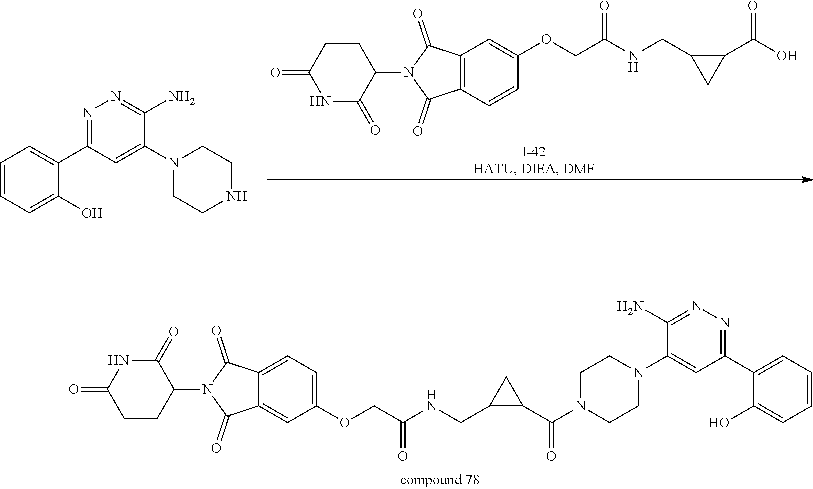 [SMILES: Nc1nnc(-c2ccccc2O)cc1N1CCNCC1 ; O=C(COc1ccc2c(c1)C(=O)N(C1CCC(=O)NC1=O)C2=O)NCC1CC1C(=O)O ; "I-42HATU, DIEA, DMF" ; Nc1nnc(-c2ccccc2O)cc1N1CCN(C(=O)C2CC2CNC(=O)COc2ccc3c(c2)C(=O)N(C2CCC(=O)NC2=O)C3=O)CC1]

To a stirred mixture of 2-[6-amino-5-(piperazin-1-yl)pyridazin-3-yl]phenol (25.60 mg, 0.094 mmol, 1.00 equiv) and 2-[(2-[[2-(2,6-dioxopiperidin-3-yl)-1,3-dioxoisoindol-5-yl]oxy]acetamido)methyl]cyclopropane-1-carboxylic acid (I-42, 44.56 mg, 0.104 mmol, 1.10 equiv) in DMF (1.4 mL) were added HATU (43.05 mg, 0.113 mmol, 1.20 equiv) and DIEA (60.97 mg, 0.472 mmol, 5.00 equiv) in portions at room temperature. The mixture was stirred for 1 h, then purified by reversed-phase preparative HPLC to afford compound 78 (21 mg, 31.53%) as an off-white solid. ¹H NMR (300 MHz, DMSO-d6) δ 14.07 (brs, 1H), 11.13 (s, 1H), 8.10-7.62 (m, 3H), 7.62-7.04 (m, 4H), 6.87 (dd, J=12.4, 7.5 Hz, 2H), 6.44 (s, 2H), 5.11 (dd, J=12.5, 5.5 Hz, 1H), 4.77 (s, 2H), 3.83 (d, J=18.8 Hz, 3H), 3.62-3.11 (s, 8H), 3.25-2.69 (m, 1H), 2.10 (d, J=12.4 Hz, 3H), 1.53 (s, 1H), 0.95 (d, J=25.2 Hz, 2H). LCMS (ESI) m/z: [M+H]+=683.30.

### Example 16. Preparation of N-[(2-[4-[3-amino-6-(2-hydroxyphenyl)pyridazin-4-yl]piperazine-1-carbonyl]cyclopropyl)methyl]-2-[[2-(2,6-dioxopiperidin-3-yl)-1,3-dioxoisoindol-4-yl]oxy]acetamide (compound 77)

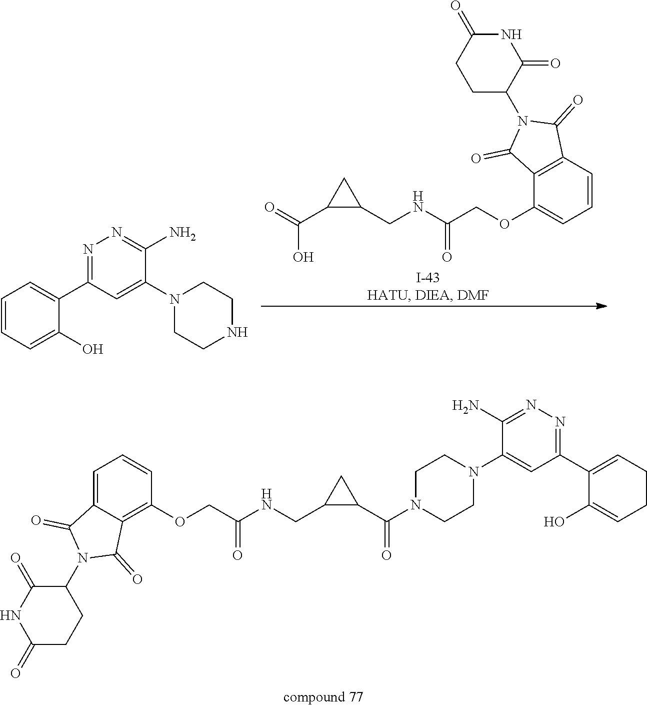

To a stirred mixture of 2-[6-amino-5-(piperazin-1-yl)pyridazin-3-yl]phenol (25.60 mg, 0.094 mmol, 1.00 equiv) and 2-[(2-[[2-(2,6-dioxopiperidin-3-yl)-1,3-dioxoisoindol-4-yl]oxy]acetamido)methyl]cyclopropane-1-carboxylic acid (I-43, 44.56 mg, 0.104 mmol, 1.10 equiv) in DMF (1.4 mL) were added HATU (43.05 mg, 0.113 mmol, 1.20 equiv) and DIEA (60.97 mg, 0.472 mmol, 5.00 equiv) in portions at room temperature. The mixture was stirred for 1 h under an atmosphere of dry nitrogen, then directly purified by reversed-phase preparative HPLC to afford compound 77 (12.5 mg, 18.73%) as a white solid. ¹H NMR (300 MHz, DMSO-d6) δ 14.14 (s, 1H), 11.12 (s, 1H), 8.29 (s, 1H), 7.84 (d, J=8.0 Hz, 1H), 7.72 (d, J=8.2 Hz, 1H), 7.45 (s, 1H), 7.36 (d, J=2.2 Hz, 1H), 7.25 (dd, J=21.1, 8.1 Hz, 2H), 6.87 (d, J=8.1 Hz, 2H), 6.41 (s, 2H), 5.10 (dd, J=12.9, 5.4 Hz, 1H), 4.71 (s, 2H), 3.88 (s, 3H), 3.68 (s, 1H), 3.57-3.42 (m, 3H), 3.31-3.14 (m, 4H), 2.95-2.80 (m, 1H), 2.68 (d, J=30.8 Hz, 1H), 2.10 (d, J=8.6 Hz, 2H), 1.55 (q, J=7.4 Hz, 1H), 0.99 (d, J=5.3 Hz, 1H), 0.95-0.74 (m, 1H). LCMS (ESI) m/z: [M+H]+=683.15.

### Example 17. Preparation of (2S,4R)-1-((S)-2-(10-((4-(2-((3-amino-6-(2-hydroxyphenyl)pyridazin-4-yl)oxy)ethyl)benzyl)amino)decanamido)-3,3-dimethylbutanoyl)-4-hydroxy-N-(4-(4-methylthiazol-5-yl)benzyl)pyrrolidine-2-carboxamide (compound 26)

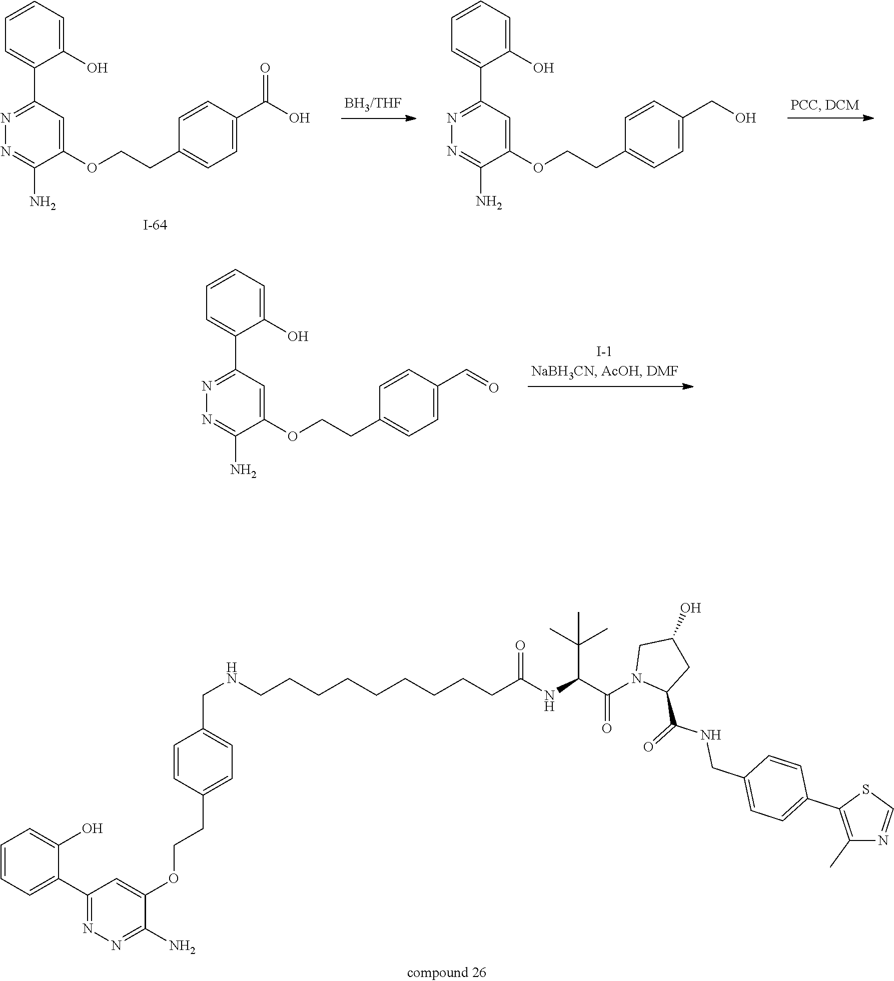

### Step 1: Preparation of 2-(6-amino-5-(4-(hydroxymethyl)phenethoxy)pyridazin-3-yl)phenol

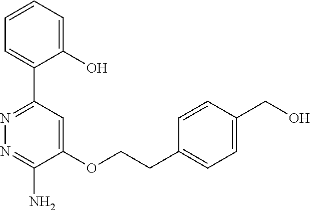 [SMILES: Nc1nnc(-c2ccccc2O)cc1OCCc1ccc(CO)cc1]

To a solution of 4-(2-((3-amino-6-(2-hydroxyphenyl)pyridazin-4-yl)oxy)ethyl)benzoic acid (I-64, 100.00 mg, 0.28 mmol, 1.00 equiv) in THF (5 mL) was added a solution of borane in THF (10.00 mL, 104.49 mmol, 367.14 equiv). This was stirred at 25° C. for 8 h and quenched with water. The mixture was filtered and the filter cake was washed with water (10 mL) to give 2-(6-amino-5-(4-(hydroxymethyl)phenethoxy)pyridazin-3-yl)phenol (87.0 mg, 90%) as a brown solid. This material was used in the next step without further purification. LCMS (ESI) m/z: [M+H]⁺=337.10.

### Step 2: Preparation of 4-(2-((3-amino-6-(2-hydroxyphenyl)pyridazin-4-yl)oxy)ethyl)benzaldehyde

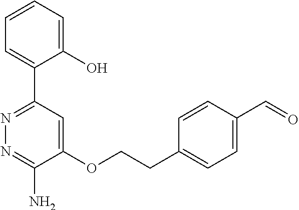 [SMILES: Nc1nnc(-c2ccccc2O)cc1OCCc1ccc(C=O)cc1]

A mixture of 2-(6-amino-5-(4-(hydroxymethyl)phenethoxy)pyridazin-3-yl)phenol (50.00 mg, 0.15 mmol, 1.0 equiv) in DCM (10 mL) was prepared and DMP (12.57 mg, 0.030 mmol, 2.0 equiv) was added. The mixture stirred at 25° C. overnight, then extracted with EtOAc (3×10 mL). The combined organic layers were washed with brine (15 mL) and dried over anhydrous sodium sulfate, filtered, and concentrated to give the crude product. This was purified by chromatography on silica gel, eluting with petroleum ether/EtOAc from 50% to 100% to give 4-(2-((3-amino-6-(2-hydroxyphenyl)pyridazin-4-yl)oxy)ethyl)benzaldehyde (25.1 mg, 50%) as an off-white solid. LCMS (ESI) m/z: [M+H]⁺=336.25.

### Step 3: Preparation of (2S,4R)-1-((S)-2-(10-((4-(2-((3-amino-6-(2-hydroxyphenyl)pyridazin-4-yl)oxy)ethyl)benzyl)amino)decanamido)-3,3-dimethylbutanoyl)-4-hydroxy-N-(4-(4-methylthiazol-5-yl)benzyl)pyrrolidine-2-carboxamide (compound 26)

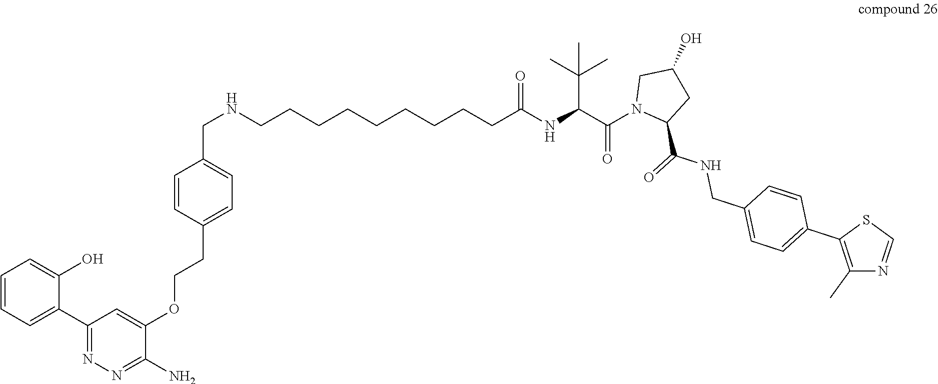 [SMILES: Cc1ncsc1-c1ccc(CNC(=O)[C@@H]2C[C@@H](O)CN2C(=O)[C@@H](NC(=O)CCCCCCCCCNCc2ccc(CCOc3cc(-c4ccccc4O)nnc3N)cc2)C(C)(C)C)cc1]

To a stirred solution of 4-(2-((3-amino-6-(2-hydroxyphenyl)pyridazin-4-yl)oxy)ethyl)benzaldehyde (25.00 mg, 0.075 mmol, 1.0 equiv) in DMF (2 mL) was added (2S,4R)-1-[(2S)-2-(10-aminodecanamido)-3,3-dimethylbutanoyl]-4-hydroxy-N-[[4-(4-methyl-1,3-thiazol-5-yl)phenyl]methyl]pyrrolidine-2-carboxamide (I-1, 49.19 mg, 0.082 mmol, 1.10 equiv) and AcOH (0.23 mg, 0.14 mmol, 0.05 equiv) under an atmosphere of dry nitrogen. The mixture was stirred for 2 hand NaBH₃CN (9.42 mg, 0.15 mmol, 2.00 equiv) was then added. The solution was stirred at room temperature for 2 h, then water (5 mL) was added and the resulting mixture was extracted with DCM (3×20 mL). The organic layers were combined and washed with brine (15 mL), then dried over anhydrous sodium sulfate, filtered, and concentrated to give a crude product which was purified by prep-HPLC to give (2S,4R)-1-((S)-2-(10-((4-(2-((3-amino-6-(2-hydroxyphenyl)pyridazin-4-yl)oxy)ethyl)benzyl)amino)decanamido)-3,3-dimethylbutanoyl)-4-hydroxy-N-(4-(4-methylthiazol-5-yl)benzyl)pyrrolidine-2-carboxamide (compound 26, 10.3 mg, 15%) as a white solid. ¹H NMR (300 MHz, DMSO-d6) δ 14.37 (brs, 1H), 8.99 (s, 1H), 8.59 (t, J=8.6 Hz, 1H), 7.99 (d, J=7.2 Hz, 1H), 7.89 (d, J=7.2 Hz, 1H), 7.59 (s, 1H), 7.50-7.31 (m, 7H), 7.30-7.12 (m, 1H), 6.94-6.81 (m, 2H), 6.50 (s, 2H), 5.12 (s, 1H), 4.78-4.02 (m, 7H), 3.81 (s, 1H), 3.71-3.60 (m, 1H), 3.13 (t, J=6.7 Hz, 2H), 2.68-2.60 (m, 2H), 2.45 (s, 3H), 2.36-1.79 (m, 5H), 1.60-1.46 (m, 4H), 1.24-1.03 (m, 11H), 0.94 (s, 9H). LCMS (ESI) m/z: [M+2H]²⁺=460.45.

The following compounds in Table E3 were prepared using procedures similar to those used for the preparation of compound 26.

TABLE E3

| | | LCMS | |
|---|---|---|---|
| | | (ESI) | |
| No. | Name | m/z | ¹H NMR |
| 122 | 4-[3-([[4-(2-[[3-amino-6-(2- | 651.35 | ¹H NMR (400 MHz, DMSO-d6) δ 14.36 (s, 1H), |
| | hydroxyphenyl)pyridazin-4- | | 11.11 (s, 1H), 8.21 (s, 1H), 8.00-7.89 (m, |
| | yl]oxy]ethyl)phenyl]meth- | | 1H), 7.81 (dd, J = 8.5, 7.3 Hz, 1H), 7.59 (s, |
| | yl]amino)propoxy]- | | 1H), 7.52 (d, J = 8.5 Hz, 1H), 7.45 (d, J = 7.3 |
| | 2-(2,6-dioxopiperidin- | | Hz, 1H), 7.38-7.30 (m, 4H), 7.27-7.19 (m, |
| | 3-yl)isoindole-1,3-dione | | 1H), 6.87 (t, J = 7.3 Hz, 2H), 6.50 (s, 2H), 5.08 |
| | | | (dd, J = 12.7, 5.4 Hz, 1H), 4.44 (t, J = 6.8 Hz, |
| | | | 2H), 4.28 (t, J = 6.2 Hz, 2H), 3.75 (s, 2H), 3.12 |
| | | | (t, J = 6.8 Hz, 2H), 2.95-2.80 (m, 1H), 2.75 (t, |
| | | | J = 6.2 Hz, 2H), 2.68-2.53 (m, 1H), 2.52- |
| | | | 2.46 (m, 1H), 2.08-1.89 (m, 3H) |
| 115 | 4-[2-([[4-(2-[[3-amino-6-(2- | 637.15 | ¹H NMR (300 MHz, DMSO-d₆) δ 14.01-14.61 |
| | hydroxyphenyl)pyridazin-4- | | (s, 1H), δ 11.11 (s, 1H), 8.22 (s, 1H), 7.99- |
| | yl]oxy]ethyl)phenyl]meth- | | 7.91 (m, 1H), 7.81 (m, 1H), 7.60 (s, 1H), 7.53 |
| | yl]amino)ethoxy]- | | (d, J = 8.6 Hz, 1H), 7.46 (d, J = 7.2 Hz, 1H), |
| | 2-(2,6-dioxopiperidin- | | 7.39-7.28 (m, 4H), 7.23 (m, 1H), 6.87 (m, |
| | 3-yl)isoindole-1,3-dione | | 2H), 6.51 (s, 2H), 5.09 (m, 1H), 4.45 (t, J = 6.9 |
| | | | Hz, 2H), 4.29 (t, J = 5.6 Hz, 2H), 3.80 (s, 2H), |
| | | | 3.13 (t, J = 6.9 Hz, 2H), 2.96-2.82 (m, 3H), |
| | | | 2.62 (s, 1H), 2.37 (m, 1H), 2.03 (m, 1H). |
| 112 | 4-[[5-([[4-(2-[[3-amino-6-(2- | 678.45 | ¹H NMR (400 MHz, DMSO-d6) δ 14.32 (s, |
| | hydroxyphenyl)pyridazin-4- | | 1H), 11.10 (s, 1H), 8.30 (s, 2H), 7.93 (d, J = 7.8 |
| | yl]oxy]ethyl)phenyl]meth- | | Hz, 1H), 7.62-7.54 (m, 2H), 7.39-7.28 (m, |
| | yl]amino)pentyl]amino]- | | 4H), 7.28-7.19 (m, 1H), 7.09 (d, J = 8.6 Hz, |
| | 2-(2,6-dioxopiperidin- | | 1H), 7.02 (d, J = 7.0 Hz, 1H), 6.87 (t, J = 7.8 |
| | 3-yl)isoindole-1,3-dione | | Hz, 2H), 6.56-6.46 (m, 3H), 5.05 (dd, J = |
| | | | 12.8, 5.4 Hz, 1H), 4.45 (t, J = 6.8 Hz, 2H), 3.74 |
| | | | (s, 2H), 3.51 (s, 1H), 3.29 (s, 2H), 3.13 (t, J = |
| | | | 6.8 Hz, 2H), 2.92-2.88 (m, 1H), 2.71-2.53 |
| | | | (m, 4H), 2.03 (ddt, J = 12.7, 7.6, 4.2 Hz, 1H), |
| | | | 1.54 (dp, J = 27.9, 7.3 Hz, 4H), 1.42-1.29 (m, |
| | | | 2H). |
| 107 | 4-[[4-([[4-(2-[[3-amino-6-(2- | 664.1 | ¹H NMR (400 MHz, DMSO-d6) δ 14.34 (s, 1H), |
| | hydroxyphenyl)pyridazin-4- | | 11.09 (s, 1H), 8.18 (s, 1H), 7.96-7.90 (m, |
| | yl]oxy]ethyl)phenyl]meth- | | 1H), 7.65-7.50 (m, 2H), 7.40 (d, J = 7.9 Hz, |
| | yl]amino)butyl]amino]- | | 2H), 7.35 (d, J = 7.9 Hz, 2H), 7.23 (td, J = 7.6, |
| | 2-(2,6-dioxopiperidin- | | 1.6 Hz, 1H), 7.10 (d, J = 8.7 Hz, 1H), 7.02 (d, |
| | 3-yl)isoindole-1,3-dione | | J = 7.0 Hz, 1H), 6.91-6.83 (m, 2H), 6.57 (t, J = |
| | | | 6.0 Hz, 1H), 6.49 (s, 2H), 5.05 (dd, J = 12.8, |
| | | | 5.4 Hz, 1H), 4.46 (t, J = 6.7 Hz, 2H), 3.87 (s, |
| | | | 2H), 3.14 (t, J = 6.7 Hz, 2H), 2.95-2.83 (m, |
| | | | 1H), 2.71 (s, 2H), 2.63-2.51 (m, 2H), 2.10- |
| | | | 1.95 (m, 1H), 1.60 (s, 4H). |
| 108 | 4-[[3-([[4-(2-[[3-amino-6-(2- | 650.2 | ¹H NMR (400 MHz, DMSO-d6) δ 14.35 (s, 1H), |
| | hydroxyphenyl)pyridazin-4- | | 11.10 (s, 1H), 8.22 (s, 1H), 7.97-7.90 (m, |
| | yl]oxy]ethyl)phenyl]meth- | | 1H), 7.63-7.54 (m, 2H), 7.35 (q, J = 8.0 Hz, |
| | yl]amino)propyl]amino]- | | 4H), 7.23 (td, J = 7.6, 1.5 Hz, 1H), 7.11 (d, J = |
| | 2-(2,6-dioxopiperidin- | | 8.6 Hz, 1H), 7.02 (d, J = 7.0 Hz, 1H), 6.87 (dd, |
| | 3-yl)isoindole-1,3-dione, | | J = 8.1, 6.8 Hz, 2H), 6.76 (t, J = 6.0 Hz, 1H), |
| | formic acid salt | | 6.50 (s, 2H), 5.05 (dd, J = 12.9, 5.4 Hz, 1H), |
| | | | 4.45 (t, J = 6.8 Hz, 2H), 3.75 (d, J = 15.0 Hz, |
| | | | 2H), 3.51 (s, 2H), 3.13 (t, J = 6.8 Hz, 2H), 2.88 |
| | | | (ddd, J = 17.6, 13.9, 5.2 Hz, 1H), 2.68-2.55 |
| | | | (m, 2H), 2.54 (s, 2H), 2.07-1.98 (m, 1H), 2.10- |
| | | | 2.09 (m, 1H), 1.77 (t, J = 6.8 Hz, 2H). |
| 109 | 4-[[2-([[4-(2-[[3-amino-6-(2- | 636.2 | ¹H NMR (400 MHz, DMSO-d6) δ 14.36 (s, 1H), |
| | hydroxyphenyl)pyridazin-4- | | 11.11 (d, J = 8.1 Hz, 1H), 8.19 (s, 1H), 7.93 |
| | yl]oxy]ethyl)phenyl]meth- | | (ddd, J = 8.2, 4.6, 1.6 Hz, 1H), 7.61-7.47 (m, |
| | yl]amino)ethyl]amino]- | | 2H), 7.37-7.26 (m, 4H), 7.23 (ddd, J = 8.5, |
| | 2-(2,6-dioxopiperidin- | | 7.1, 1.6 Hz, 1H), 7.11-6.93 (m, 2H), 6.91- |
| | 3-yl)isoindole-1,3-dione, | | 6.72 (m, 3H), 6.49 (d, J = 5.7 Hz, 2H), 5.08 (td, |
| | formic acid salt | | J = 13.5, 5.4 Hz, 1H), 4.43 (q, J = 7.1 Hz, 2H), |
| | | | 3.72 (s, 1H), 3.58 (d, J = 3.6 Hz, 1H), 3.36 (d, |
| | | | J = 6.4 Hz, 2H), 3.10 (dt, J = 9.2, 6.9 Hz, 2H), |
| | | | 2.89 (ddd, J = 17.4, 13.5, 5.7 Hz, 1H), 2.73 (t, |
| | | | J = 6.0 Hz, 1H), 2.64-2.52 (m, 2H), 2.45 (t, J = |
| | | | 7.2 Hz, 1H), 1.50 (d, J = 7.8 Hz, 1H), 1.41 (q, |
| | | | J = 7.5, 6.8 Hz, 1H). |
| 113 | 4-((5-((4-(2-((3-amino-6-(2- | 693.5 | 1H NMR (400 MHz, DMSO-d6) δ 14.32 (s, 1H), |
| | hydroxyphenyl)pyridazin-4- | | 11.09 (s, 1H), 8.15 (s, 1H), 7.93 (dd, J = 7.9, |
| | yl)oxy)ethyl)benzyl)(meth- | | 1.6 Hz, 1H), 7.80 (dd, J = 8.5, 7.3 Hz, 1H), |
| | yl)amino)pentyl)oxy)- | | 7.59 (s, 1H), 7.50 (d, J = 8.5 Hz, 1H), 7.43 (d, |
| | 2-(2,6-dioxopiperidin- | | J = 7.2 Hz, 1H), 7.33 (d, J = 7.8 Hz, 2H), 7.24 |
| | 3-yl)isoindoline-1,3-dione | | (dd, J = 7.7, 2.1 Hz, 2H), 6.92-6.82 (m, 2H), |
| | | | 6.48 (s, 2H), 5.07 (dd, J = 12.9, 5.3 Hz, 1H), |
| | | | 4.45 (t, J = 6.9 Hz, 2H), 4.19 (t, J = 6.4 Hz, |
| | | | 2H), 3.44 (s, 3H), 3.12 (t, J = 6.8 Hz, 2H), 2.88 |
| | | | (ddd, J = 18.5, 14.2, 5.5 Hz, 1H), 2.71-2.53 |
| | | | (m, 2H), 2.33 (d, J = 8.6 Hz, 2H), 2.09 (d, J = |
| | | | 1.9 Hz, 2H), 2.07-1.96 (m, 1H), 1.75 (p, J = |
| | | | 6.7 Hz, 2H), 1.61-1.37 (m, 4H). |
| 116 | 5-((5-((4-(2-((3-amino-6-(2- | 679.5 | ¹H NMR (300 MHz, DMSO-d6) δ 14.30 (s, 1H), |
| | hydroxyphenyl)pyridazin-4- | | 11.10 (s, 1H), 8.34 (s, 2H, FA), 7.98-7.89 (m, |
| | yl)oxy)ethyl)benzyl)amino)pentyl)oxy)- | | 1H), 7.83 (d, J = 8.3 Hz, 1H), 7.59 (s, 1H), 7.42 |
| | 2-(2,6-dioxopiperidin-3- | | (d, J = 2.2 Hz, 1H), 7.39-7.28 (m, 5H), 7.27- |
| | yl)isoindoline-1,3-dione; | | 7.18 (m, 1H), 6.92-6.91 (m, 2H), 6.49 (s, 2H), |
| | diformic acid | | 5.12 (dd, J = 12.8, 5.4 Hz, 1H), 4.46 (t, J = 6.9 |
| | | | Hz, 2H), 4.17 (t, J = 6.4 Hz, 2H), 3.73 (s, 2H), |
| | | | 3.13 (t, J = 6.8 Hz, 2H), 2.96-2.81 (m, 1H), |
| | | | 2.66-2.53 (m, 4H), 2.10-2.00 (m, 1H), 1.82- |
| | | | 1.67 (m, 2H), 1.60-1.36 (m, 4H). |
| 117 | 5-[[5-([[4-(2-[[3-amino-6-(2- | 678.4 | ¹H NMR (300 MHz, DMSO-d6) δ 11.06 (s, 1H), |
| | hydroxyphenyl)pyridazin-4- | | 8.19 (s, 1H), 7.99-7.89 (m, 1H), 7.63-7.52 |
| | yl]oxy]ethyl)phenyl]meth- | | (m, 2H), 7.48-7.34 (m, 4H), 7.24 (td, J = 7.5, |
| | yl]amino)pentyl]amino]- | | 1.6 Hz, 1H), 7.12 (t, J = 5.3 Hz, 1H), 6.95 (d, |
| | 2-(2,6-dioxopiperidin- | | J = 2.1 Hz, 1H), 6.92-6.80 (m, 3H), 6.50 (s, |
| | 3-yl)isoindole-1,3-dione | | 2H), 5.03 (dd, J = 12.8, 5.4 Hz, 1H), 4.47 (t, J = |
| | | | 6.7 Hz, 2H), 3.93 (s, 2H), 3.15 (t, J = 6.2 Hz, |
| | | | 4H), 2.97-2.79 (m, 1H), 2.73 (t, J = 7.4 Hz, |
| | | | 2H), 2.61 (s, 1H), 2.55 (s, 1H), 2.04-1.94 (m, |
| | | | 1H), 1.58 (s, 4H), 1.41 (d, J = 7.9 Hz, 2H). |
| 127 | 4-[3-(1-[[4-(2-[[3-amino-6-(2- | 691.2 | ¹H NMR (400 MHz, DMSO-d6) δ 14.35 (s, 1H), |
| | hydroxyphenyl)pyridazin-4- | | 11.10 (s, 1H), 8.16 (s, 1H, FA salt), 7.94 (d, J = |
| | yl]oxy]ethyl)phenyl]meth- | | 8.0 Hz, 1H), 7.85-7.76 (m, 1H), 7.59 (s, 1H), |
| | yl]azetidin-3-yl)propoxy]- | | 7.50 (d, J = 8.5 Hz, 1H), 7.44 (d, J = 7.2 Hz, |
| | 2-(2,6-dioxopiperidin- | | 1H), 7.33 (d, J = 7.8 Hz, 2H), 7.27-7.18 (m, |
| | 3-yl)isoindole-1,3-dione | | 3H), 6.87 (d, J = 8.0 Hz, 2H), 6.50 (s, 2H), 5.08 |
| | | | (dd, J = 12.9, 5.4 Hz, 1H), 4.44 (t, J = 6.9 Hz, |
| | | | 2H), 4.18 (s, 2H), 3.54 (s, 2H), 3.41-3.35 (m, |
| | | | 2H), 3.12 (t, J = 6.9 Hz, 2H), 2.90-2.81 (m, |
| | | | 1H), 2.81 (t, 2H), 2.64-2.53 (m, 1H), 2.48- |
| | | | 2.36 (m, 2H), 2.06-1.99 (m, 1H), 1.71-1.65 |
| | | | (m, 4H). |
| 126 | 4-[2-(4-[[4-(2-[[3-amino-6-(2- | 706.3 | ¹H NMR (400 MHz, DMSO-d6) δ 11.11 (s, 1H), |
| | hydroxyphenyl)pyridazin-4- | | 9.86 (br s, 1H), 7.93-7.69 (m, 2H), 7.63 (s, |
| | yl]oxy]ethyl)phenyl]meth- | | 1H), 7.58-7.23 (m, 7H), 7.23-6.86 (m, 4H), |
| | yl]piperazin-1-yl)ethoxy]- | | 5.09 (dd, J = 12.8, 5.4 Hz, 1H), 4.78-4.01 (m, |
| | 2-(2,6-dioxopiperidin- | | 4H), 4.02-3.47 (m, 4H), 3.34-3.23 (m, 4H), |
| | 3-yl)isoindole-1,3-dione | | 3.18 (t, J = 6.8 Hz, 4H), 2.94-2.82 (m, 1H), |
| | | | 2.69-2.53 (m, 3H), 2.46 (d, J = 5.4 Hz, 1H), |
| | | | 2.07-1.97 (m, 1H). |
| 120 | 3-(5-[[5-([[4-(2-[[3-amino-6-(2- | 678.4 | ¹H NMR (400 MHz, DMSO-d6) δ 14.35 (s, 1H), |
| | hydroxyphenyl)pyridazin-4- | | 11.17 (s, 1H), 8.19 (m, 1H), 7.96-7.89 (m, |
| | yl]oxy]ethyl)phenyl]meth- | | 1H), 7.80 (t, J = 8.1 Hz, 1H), 7.59 (s, 1H), 7.42 |
| | yl]amino)pentyl]amino]- | | (d, J = 7.9 Hz, 2H), 7.37 (d, J = 7.9 Hz, 2H), |
| | 4-oxo-1,2,3-benzotriazin- | | 7.23 (m, 2H), 6.96 (d, J = 8.5 Hz, 1H), 6.88 (m, |
| | 3-yl)piperidine-2,6-dione | | 2H), 6.50 (s, 2H), 5.85 (dd, J = 12.2, 5.4 Hz, |
| | | | 1H), 4.46 (t, J = 6.7 Hz, 2H), 3.93 (s, 2H), 3.26- |
| | | | 3.18 (m, 2H), 3.15 (t, J = 6.7 Hz, 2H), 2.99- |
| | | | 2.86 (m, 1H), 2.74 (m, 2H), 2.66 (d, J = 14.8 |
| | | | Hz, 1H), 2.45-2.31(m, 2H), 2.23 (m, 1H), 1.60 |
| | | | (m, 4H), 1.44-1.36 (m, 2H). |
| 121 | 4-(4-((4-(2-((3-amino-6-(2- | 665.2 | ¹H NMR (400 MHz, DMSO-d6) δ 14.35 (s, 1H), |
| | hydroxyphenyl)pyridazin-4- | | 11.08 (s, 1H), 8.17 (s, 1H), 7.96-7.89 (m, |
| | yl)oxy)ethyl)benzyl)amino)butoxy)- | | 1H), 7.81 (dd, J = 8.5, 7.3 Hz, 1H), 7.58 (s, |
| | 2-(2,6-dioxopiperidin-3- | | 1H), 7.50 (d, J = 8.5 Hz, 1H), 7.45 (d, J = 7.2 |
| | yl)isoindoline-1,3-dione | | Hz, 1H), 7.45-7.40 (m, 2H), 7.38-7.32 (m, |
| | | | 2H), 7.23 (td, J = 7.6, 1.6 Hz, 1H), 6.92-6.82 |
| | | | (m, 2H), 6.48 (s, 2H), 5.07 (dd, J = 12.6, 5.5 |
| | | | Hz, 1H), 4.45 (t, J = 6.7 Hz, 2H), 4.21 (t, J = |
| | | | 6.1 Hz, 2H), 3.87 (s, 2H), 3.13 (t, J = 6.7 Hz, |
| | | | 2H), 2.88 (ddd, J = 16.7, 13.6, 5.3 Hz, 1H), |
| | | | 2.78 (d, J = 7.6 Hz, 2H), 2.70-2.53 (m, 1H), |
| | | | 2.42-2.27 (m, 1 H), 2.12-1.94 (m, 1H), 1.81 |
| | | | (t, J = 7.1 Hz, 2H), 1.71 (d, J = 7.3 Hz, 2H). |
| 151 | 5-[[5-([[4-(2-[[3-amino-6-(2- | 691.9 | ¹H NMR (400 MHz, DMSO-d₆) δ 7.93 (dd, J = |
| | hydroxyphenyl)pyridazin-4- | | 8.0, 1.6 Hz, 1H), 7.59 (s, 1H), 7.55 (d, J = 8.4 |
| | yl]oxy]ethyl)phenyl]meth- | | Hz, 1H), 7.36-7.29 (m, 2H), 7.27-7.18 (m, |
| | yl](methyl)amino)pentyl]amino]- | | 3H), 7.14-7.07 (m, 1H), 6.97-6.92 (m, 1H), |
| | 2-(2,6-dioxopiperidin- | | 6.92-6.80 (m, 3H), 6.50 (s, 2H), 5.02 (dd, J = |
| | 3-yl)isoindole-1,3-dione | | 12.9, 5.4 Hz, 1H), 4.45 (t, J = 6.9 Hz, 2H), 3.40 |
| | | | (s, 2H), 3.18-3.08 (m, 4H), 2.90-2.80 (m, |
| | | | 1H), 2.62-2.51 (m, 2H), 2.30 (t, J = 7.1 Hz, |
| | | | 2H), 2.07 (s, 3H), 2.04-1.93 (m, 1H), 1.62- |
| | | | 1.43 (m, 4H), 1.43-1.29 (m, 2H). |
| 156 | 4-((5-((4-(2-((3-amino-6-(2- | 692.2 | ¹H NMR (400 MHz, DMSO-d₆) δ14.36 (s, 1H), |
| | hydroxyphenyl)pyridazin-4- | | 11.09 (s, 1H), 8.20 (s, 1H), 7.93 (dd, J = 8.0, |
| | yl)oxy)ethyl)benzyl)(meth- | | 1.6 Hz, 1H), 7.61-7.53 (m, 2H), 7.36-7.29 |
| | yl)amino)pentyl)amino)- | | (m, 2H), 7.27-7.18 (m, 3H), 7.08 (d, J = 8.6 |
| | 2-(2,6-dioxopiperidin- | | Hz, 1H), 7.01 (d, J = 7.0 Hz, 1H), 6.91-6.82 |
| | 3-yl)isoindoline-1,3-dione | | (m, 2H), 6.56-6.51 (m, 1H), 6.50 (s, 2H), 5.05 |
| | formate | | (dd, J = 12.9, 5.4 Hz, 1H), 4.45 (t, J = 7.0 Hz, |
| | | | 2H), 3.40 (s, 2H), 3.31-3.23 (m, 2H), 3.12 (t, |
| | | | J = 6.9 Hz, 2H), 2.94-2.81 (m, 1H), 2.62- |
| | | | 2.53 (m, 2H), 2.34-2.26 (m, 2H), 2.07 (s, 3H), |
| | | | 2.05-1.96 (m, 1H), 1.62-1.43 (m, 4H), 1.40- |
| | | | 1.32 (m, 2H). |

### Preparation of ((2S,4R)-1-((S)-2-(10-((4-(2-((3-amino-6-(2-hydroxyphenyl)pyridazin-4-yl)(methyl)amino)ethyl)benzyl)amino)decanamido)-3,3-dimethylbutanoyl)-4-hydroxy-N-(4-(4-methylthiazol-5-yl)benzyl)pyrrolidine-2-carboxamide (compound 131)

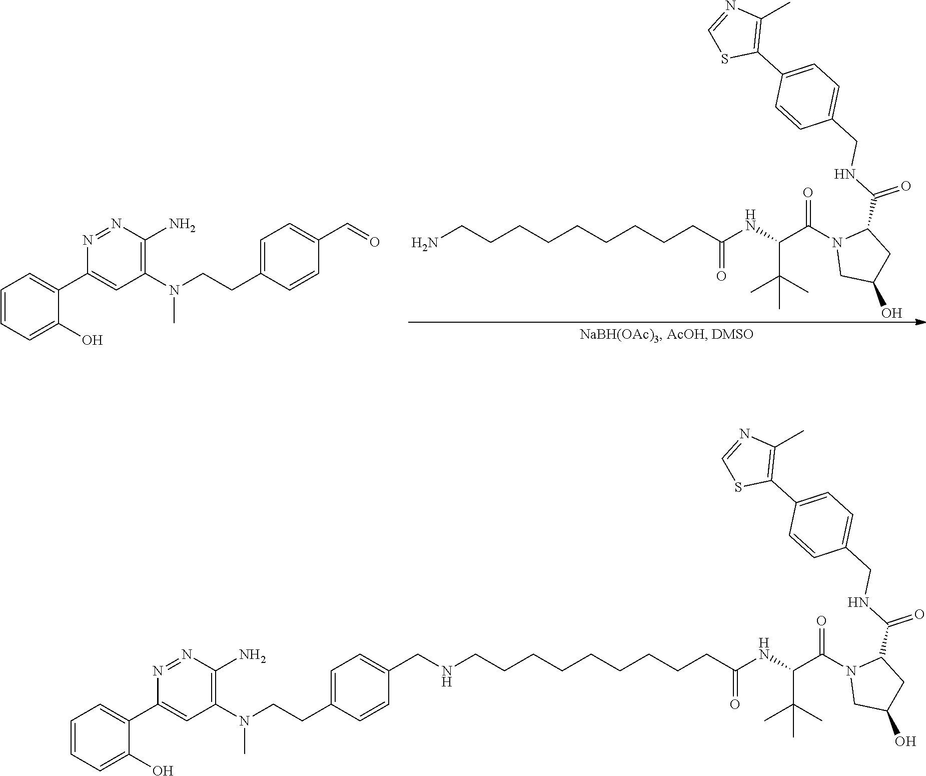

### Step 1: Preparation of (2S,4R)-1-((S)-2-(10-((4-(2-((3-amino-6-(2-hydroxyphenyl)pyridazin-4-yl)(methyl)amino)ethyl)benzyl)amino)decanamido)-3,3-dimethylbutanoyl)-4-hydroxy-N-(4-(4-methylthiazol-5-yl)benzyl)pyrrolidine-2-carboxamide (Compound 131)

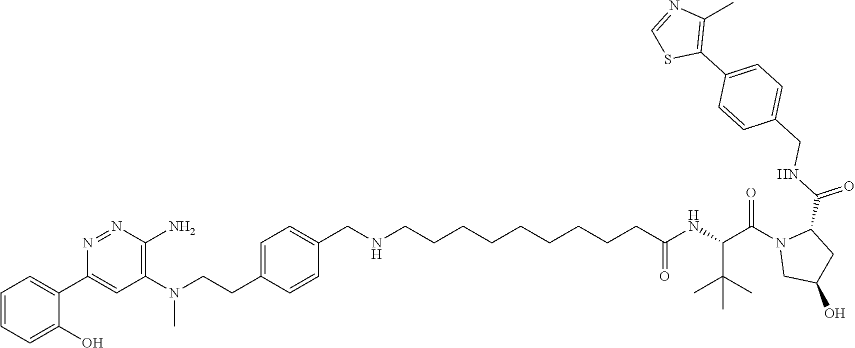 [SMILES: Cc1ncsc1-c1ccc(CNC(=O)[C@@H]2C[C@@H](O)CN2C(=O)[C@@H](NC(=O)CCCCCCCCCNCc2ccc(CCN(C)c3cc(-c4ccccc4O)nnc3N)cc2)C(C)(C)C)cc1]

To a stirred mixture of 4-(2-((3-amino-6-(2-hydroxyphenyl)pyridazin-4-yl)(methyl)amino)ethyl)benzaldehyde (20.00 mg, 0.057 mmol, 1.00 equiv) and (2S,4R)-1-[(2S)-2-(10-aminodecanamido)-3,3-dimethylbutanoyl]-4-hydroxy-N-[[4-(4-methyl-1,3-thiazol-5-yl)phenyl]methyl]pyrrolidine-2-carboxamide (41.32 mg, 0.069 mmol, 1.20 equiv) in DMSO (1.00 mL) was added AcOH (10.34 mg, 0.172 mmol, 3.00 equiv) at room temperature. The resulting mixture was stirred for 10 min at room temperature, and NaBH(OAc)₃ (73.00 mg, 0.344 mmol, 6.00 equiv) was then added. The resulting mixture was stirred overnight at room temperature, then filtered and concentrated in vacuo. The residue was purified by reverse flash chromatography Column (XBridge Shield RP18 OBD Column, 5 um, 19*150 mm; Mobile Phase A:Water (10 MMOL/L NH₄HCO₃), Mobile Phase B:ACN; Flow rate:25 mL/min; Gradient: 40 B to 50 B in 10 min; 254/220 nm; RT₁:9.03). This provided the title compound (2.1 mg, 3.62%) as a while solid. 1H NMR (400 MHz, DMSO-d6) δ 14.34 (br s, 1H), 8.98 (s, 1H), 8.56 (t, J=6.0 Hz, 1H), 7.90-7.79 (m, 2H), 7.47-7.34 (m, 5H), 7.28-7.15 (m, 5H), 6.91-6.85 (m, 2H), 6.09 (s, 2H), 5.12 (s, 1H), 4.54 (d, J=9.3 Hz, 1H), 4.48-4.39 (m, 2H), 4.35 (s, 1H), 4.21 (dd, J=15.9, 5.4 Hz, 1H), 3.80-3.58 (m, 4H), 3.47-3.35 (m, 2H), 2.94 (s, 3H), 2.80 (t, J=7.7 Hz, 2H), 2.44 (s, 4H), 2.35-1.82 (m, 5H), 1.52-1.39 (m, 3H), 1.23 (s, 11H), 0.93 (s, 9H). LCMS (ESI) m/z [M+H]⁺=932.50.

The following compounds in Table E4 were prepared using procedures similar to those used for the preparation of compound 131.

TABLE E4

| | | LCMS | |
|---|---|---|---|
| | | (ESI) | |
| No. | Name | m/z | ¹H NMR |
| 139 | (2S,4R)-1-((S)-2-(10-((4-(2-((3- | 918.5 | ¹H NMR (300 MHz, Methanol-d4) δ 8.88 (s, |
| | amino-6-(2- | | 1H), 7.63 (dd, J = 8.4, 1.6 Hz, 1H), 7.50-7.37 |
| | hydroxyphenyl)pyridazin-4- | | (m, 4H), 7.36-7.25 (m, 4H), 7.24-7.12 (m, |
| | yl)amino)ethyl)benzyl)ami- | | 1H), 6.94-6.76 (m, 3H), 4.73 (s, 1H), 4.61- |
| | no)decanamido)-3,3- | | 4.55 (m, 1H), 4.54-4.46 (m, 2H), 4.39-4.26 |
| | dimethylbutanoyl)-4-hydroxy-N- | | (m, 1H), 3.93-3.86 (m, 1H), 3.85-3.73 (m, |
| | (4-(4-methylthiazol-5- | | 3H), 3.68-3.55 (m, 2H), 3.07-2.96 (m, 2H), |
| | yl)benzyl)pyrrolidine-2- | | 2.65-2.53 (m, 2H), 2.46 (s, 3H), 2.37-2.16 |
| | carboxamide | | (m, 3H), 2.14-1.99 (m. 1H), 1.69-1.55 (m, |
| | | | 2H), 1.54-1.43 (m, 2H), 1.30 (s, 10H), 1.05 |
| | | | (s, 9H). |
| 166 | 4-(4-(2-((4-(2-((3-amino-6-(2- | 704.1 | ¹H NMR (400 MHz, DMSO-d6) δ 15.16 (s, 1H), |
| | hydroxyphenyl)pyridazin-4- | | 11.09 (s, 1H), 7.95-7.84 (m, 1H), 7.74-7.63 |
| | yl)amino)ethyl)benzyl)amino)eth- | | (m, 1H), 7.43-7.31 (m, 2H), 7.28 (s, 4H), 7.19 |
| | yl)piperazin-1-yl)-2-(2,6- | | (t, J = 7.3 Hz, 1H), 6.98 (s, 1H), 6.82 (ddd, J = |
| | dioxopiperidin-3-yl)isoindoline- | | 7.4, 3.9, 2.6 Hz, 2H), 6.31 (d, J = 5.2 Hz, 1H), |
| | 1,3-dione | | 6.27 (s, 2H), 5.09 (dd, J = 12.8, 5.3 Hz, 1H), |
| | | | 3.69 (s, 2H), 3.53 (dd, J = 13.1, 6.4 Hz, 2H), |
| | | | 3.35-3.29 (m, 4H), 2.94 (t, J = 7.5 Hz, 2H), |
| | | | 2.85 (d, J = 12.7 Hz, 1H), 2.71-2.57 (m, 3H), |
| | | | 2.54 (s, 6H), 2.47 (s, 1H), 2.11-1.93 (m, 1H). |
| 160 | 4-(4-(2-((4-(2-((3-amino-6-(2- | 718.2 | ¹H NMR (300 MHz, DMSO-d6) δ 11.10 (s, 1H), |
| | hydroxyphenyl)pyridazin-4- | | 8.31 (s, 2H), 7.93-7.84 (m, 1H), 7.77-7.64 |
| | yl)(methyl)amino)eth- | | (m, 1H), 7.36 (dd, J = 15.4, 8.3 Hz, 3H), 7.28- |
| | yl)benzyl)amino)ethyl)piperazin- | | 7.11 (m, 5H), 6.94-6.81 (m, 2H), 6.09 (s, 2H), |
| | 1-yl)-2-(2,6-dioxopiperidin-3- | | 5.09 (dd, J = 12.8, 5.4 Hz, 1H), 3.65 (s, 2H), |
| | yl)isoindoline-1,3-dione | | 3.21 (s, 7H), 2.94 (s, 3H), 2.81 (t, J = 7.8 Hz, |
| | diformate | | 4H), 2.59 (d, J = 5.9 Hz, 5H), 2.45 (s, 4H), 2.02 |
| | | | (d, J = 12.1 Hz, 1H). |
| 158 | 4-(5-(2-((4-(2-((3-amino-6-(2- | 717.3 | ¹H NMR (400 MHz, DMSO-d6) δ 11.08 (d, J = |
| | hydroxyphenyl)pyridazin-4- | | 2.3 Hz, 1H), 7.85 (d, J = 7.8 Hz, 1H), 7.64- |
| | yl)oxy)ethyl)benzyl)amino)ethyl)- | | 7.57 (m, 2H), 7.49 (d, J = 8.0 Hz, 2H), 7.43 |
| | 2,5-diazabicyclo[2.2.1]heptan- | | (dd, J = 8.5, 2.5 Hz, 2H), 7.27 (t, J = 7.7 Hz, |
| | 2-yl)-2-(2,6-dioxopiperidin-3- | | 1H), 7.20 (s, 1H), 7.15 (d, J = 8.6 Hz, 1H), 6.94- |
| | yl)isoindoline-1,3-dione | | 6.86 (m, 2H), 6.75 (s, 2H), 5.08 (dd, J = 12.6, |
| | | | 5.4 Hz, 1H), 4.93 (s, 1H), 4.49 (t, J = 6.5 Hz, |
| | | | 2H), 4.14 (s, 2H), 4.00 (s, 1H), 3.51 (s, 4H), |
| | | | 3.25 (s, 4H), 3.17 (t, J = 6.5 Hz, 3H), 2.91- |
| | | | 2.83 (m, 1H), 2.64-2.55 (m, 1H), 2.14-2.00 |
| | | | (m, 3H). |
| 143 | 4-(4-(1-(4-(2-((3-amino-6-(2- | 717.3 | ¹H NMR (400 MHz, DMSO-d6) δ 14.35 (s, 1H), |
| | hydroxyphenyl)pyridazin-4- | | 11.09 (s, 1H), 7.97-7.90 (m, 1H), 7.71 (dd, |
| | yl)oxy)ethyl)benzyl)azetidin-3- | | J = 8.4, 7.2 Hz, 1H), 7.59 (s, 1H), 7.43 (d, J = 7.7 |
| | yl)piperazin-1-yl)-2-(2,6- | | Hz, 2H), 7.40-7.31 (m, 4H), 7.24 (td, J = 7.6, |
| | dioxopiperidin-3-yl)isoindoline- | | 1.6 Hz, 1H), 6.91-6.83 (m, 2H), 6.51 (s, 2H), |
| | 1,3-dione | | 5.08 (dd, J = 12.7, 5.4 Hz, 1H), 4.47 (t, J = 6.7 |
| | | | Hz, 2H), 3.99 (s, 2H), 3.77 (s, 2H), 3.51 (s, |
| | | | 2H), 3.33 (s, 5H), 3.15 (t, J = 6.7 Hz, 2H), 2.94- |
| | | | 2.80 (m, 1H), 2.64-2.50 (m, 2H), 2.49 (s, |
| | | | 4H), 2.02 (dd, J = 10.0, 5.1 Hz, 1H). |
| 261 | (2S,4R)-1-((S)-2-(15-((4-(2-((3- | 1003.70 | ¹H NMR (300 MHz, DMSO-d6) δ 14.39 (s, 1H), |
| | amino-6-(2- | | 8.99 (s, 1H), 8.38 (d, J = 7.8 Hz, 1H), 8.00- |
| | hydroxyphenyl)pyridazin-4- | | 7.89 (m, 1H), 7.79 (d, J = 9.2 Hz, 1H), 7.60 (s, |
| | yl)oxy)ethyl)benzyl)amino)penta- | | 1H), 7.44 (d, J = 8.3 Hz, 2H), 7.41-7.30 (m, |
| | decanamido)-3,3- | | 4H), 7.30-7.15 (m, 3H), 6.96-6.73 (m, 2H), |
| | dimethylbutanoyl)-4-hydroxy-N- | | 6.51 (s, 2H), 5.11 (d, J = 3.5 Hz, 1H), 5.00- |
| | ((S)-1-(4-(4-methylthiazol-5- | | 4.78 (m, 1H), 4.59-4.33 (m, 4H), 4.28 (s, 1H), |
| | yl)phenyl)ethyl)pyrrolidine-2- | | 3.73-3.45 (m, 4H), 3.12 (t, J = 6.9 Hz, 2H), |
| | carboxamide | | 2.46-2.40 (m, 6H), 2.23 (t, J = 7.4 Hz, 1H), |
| | | | 2.15-1.89 (m, 2H), 1.80 (td, J = 8.4, 4.3 Hz, |
| | | | 1H), 1.58-1.42(m, 3H), 1.38 (d, J = 7.0 Hz, 4H), |
| | | | 1.23 (s, 20H), 0.94 (s, 9H). |

### Preparation of N′-[[4-(2-[[3-amino-6-(2-hydroxyphenyl)pyridazin-4-yl](methyl)amino]ethyl)phenyl]methyl]-N-[(2S)-1-[(2S,4R)-4-hydroxy-2-([[4-(4-methyl-1,3-thiazol-5-yl)phenyl]methyl]carbamoyl)pyrrolidin-1-yl]-3,3-dimethyl-1-oxobutan-2-yl]decanediamide (compound 132)

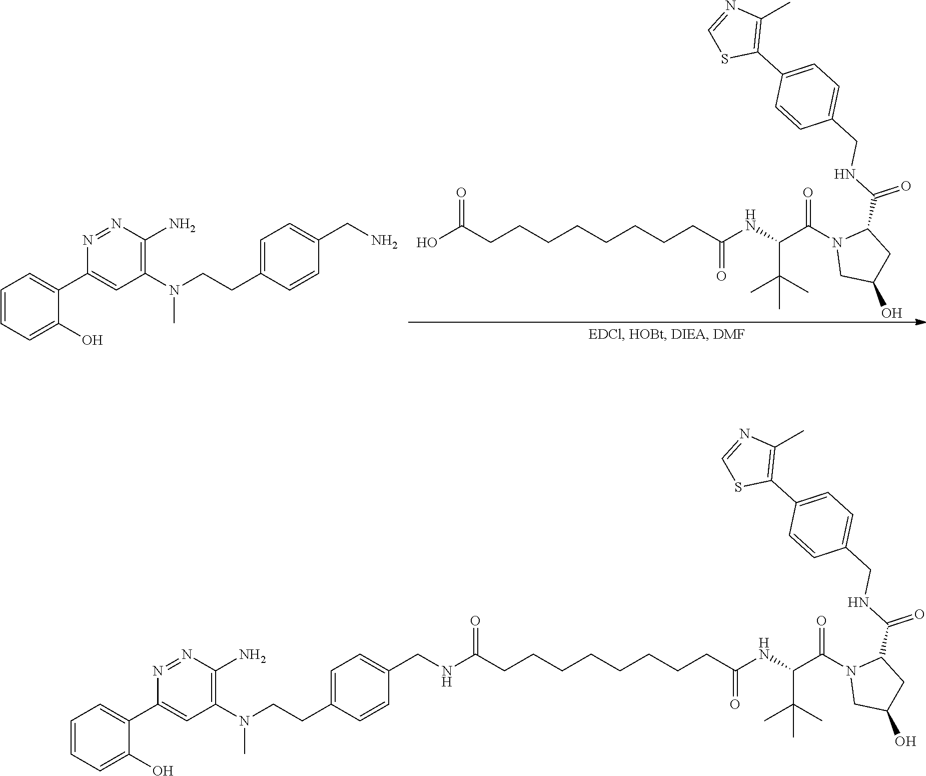

### Step 1: Preparation of N′-[[4-(2-[[3-amino-6-(2-hydroxyphenyl)pyridazin-4-yl](methyl)amino]ethylphenyl]methyl]-N-[(2S)-1-[(2S,4R)-4-hydroxy-2-([[4-(4-methyl-1,3-thiazol-5-yl)phenyl]methyl]carbamoyl)pyrrolidin-1-yl]-3,3-dimethyl-1-oxobutan-2-yl]decanediamide (Compound 132)

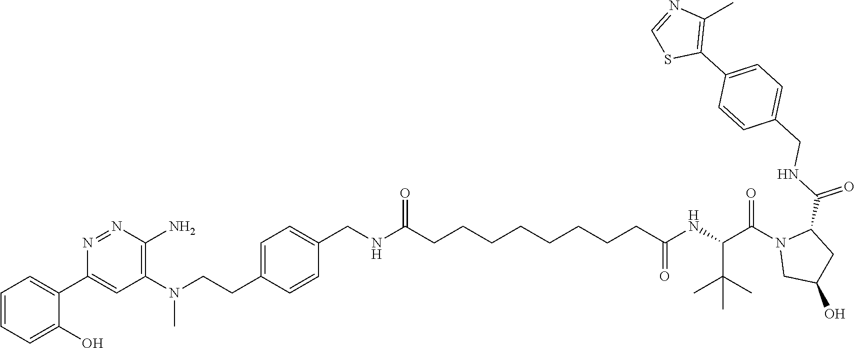 [SMILES: Cc1ncsc1-c1ccc(CNC(=O)[C@@H]2C[C@@H](O)CN2C(=O)[C@@H](NC(=O)CCCCCCCCC(=O)NCc2ccc(CCN(C)c3cc(-c4ccccc4O)nnc3N)cc2)C(C)(C)C)cc1]

A solution of 10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecanoic acid (9.99 mg, 0.016 mmol, 1.00 equiv), EDCI (6.23 mg, 0.033 mmol, 2.00 equiv), HOBt (4.39 mg, 0.033 mmol, 2.00 equiv), and DIEA (6.30 mg, 0.049 mmol, 3.00 equiv) in DMF (1.00 mL) was stirred for 20 min at 25 degrees C. 2-[6-amino-5-([2-[4-(aminomethyl)phenyl]ethyl](methyl)amino)pyridazin-3-yl] phenol (5.68 mg, 0.016 mmol, 1.00 equiv) in DMF (0.5 mL) was then added dropwise at 25 degrees C. The resulting mixture was stirred for 3 h at 25 degrees C. The reaction mixture was filtered and concentrated in vacuo. The residue was purified by prep-HPLC (Column: XSelect CSH Prep C18 OBD Column, 19*250 mm, 5 um; Mobile Phase A:Water (0.05% FA), Mobile Phase B:ACN; Flow rate: 25 mL/min.) to afford N′-[[4-(2-[[3-amino-6-(2-hydroxyphenyl)pyridazin-4-yl](methyl)amino]ethylphenyl]methyl]-N-[(2S)-1-[(2S,4R)-4-hydroxy-2-([[4-(4-methyl-1,3-thiazol-5-yl)phenyl]methyl]carbamoyl)pyrrolidin-1-yl]-3,3-dimethyl-1-oxobutan-2-yl]decanediamide (compound 132, 4.2 mg, 27.31%) as a white solid. 1H NMR (300 MHz, DMSO-d6) δ 14.34 (brs, 1H), 8.99 (s, 1H), 8.57 (t, J=6.0 Hz, 1H), 8.20 (t, J=5.9 Hz, 1H), 7.95-7.80 (m, 2H), 7.48-7.34 (m, 5H), 7.30-7.09 (m, 5H), 6.95-6.83 (m, 2H), 6.10 (s, 2H), 5.13 (s, 1H), 4.55-4.35 (m, 4H), 4.25-4.21 (m, 3H), 3.66 (s, 2H), 3.39 (d, J=8.3 Hz, 2H), 2.94 (s, 3H), 2.79 (t, J=7.8 Hz, 2H), 2.45 (s, 3H), 2.34-2.18 (m, 1H), 2.11-2.01 (m, 4H), 1.97-1.83 (m, 1H), 1.49 (s, 4H), 1.27-1.20 (m, 8H), 0.94 (s, 9H). LCMS (ESI) m/z [M+H]+=946.45.

The following compounds in Table E5 were prepared using procedures similar to those used for the preparation of compound 132.

TABLE E5

| | | LCMS | |
|---|---|---|---|
| | | (ESI) | |
| No. | Name | m/z | ¹H NMR |
| 133 | N1-(4-(2-((3-amino-6-(2- | 1044.6 | 1H NMR (300 MHz, DMSO-d6) δ 8.99 (s, 1H), |
| | hydroxyphenyl)pyridazin-4- | | 8.62-8.50 (m, 1H), 8.25-8.11 (m, 1H), 7.85 |
| | yl)(methyl)amino)ethyl)benzyl)- | | (d, J = 9.3 Hz, 1H), 7.66 (brs, 1H), 7.39-7.36 |
| | N17-((S)-1-((2S,4R)-4-hydroxy- | | (m, 6H), 7.19-7.10 (m, 4H), 6.96 (t, J = 8.1 |
| | 2-((4-(4-methylthiazol-5- | | Hz, 2H), 6.54 (brs, 1H), 5.13 (s, 1H), 4.59- |
| | yl)benzyl)carbamoyl)pyrrolidin- | | 4.32 (m, 4H), 4.21 (dd, J = 21.4, 5.7 Hz, 3H), |
| | 1-yl)-3,3-dimethyl-1-oxobutan- | | 3.66-3.61 (m, 4H), 3.05 (s, 3H), 2.85 (dd, J = |
| | 2-yl)heptadecanediamide | | 18.0, 9.9 Hz, 2H), 2.45 (s, 3H), 2.27-2.24 (m, |
| | | | 1H), 2.08 (q, J = 11.0, 9.1 Hz, 4H), 2.03-1.83 |
| | | | (m, 1H), 1.48 (s, 4H), 1.28-1.26 (m, 22H), |
| | | | 0.94 (s, 9H). |
| 136 | N′-[[4-(2-[[3-amino-6-(2- | 1030.4 | ¹H NMR (400 MHz, DMSO-d6) δ 8.98 (s, 1H), |
| | hydroxyphenyl)pyridazin-4- | | 8.55 (t, J = 6.0 Hz, 1H), 8.24 (t, J = 6.0 Hz, |
| | yl]amino]ethyl)phenyl]methyl]- | | 1H), 7.94-7.75 (m, 2H), 7.52-7.33 (m, 4H), |
| | N-[(2S)-1-[(2S,4R)-4-hydroxy- | | 7.32-7.12 (m, 5H), 6.99 (s, 1H), 6.86-6.77 |
| | 2-([[4-(4-methyl-1,3-thiazol-5- | | (m, 2H), 6.45-6.11 (m, 3H), 5.11 (s, 1H), 4.54 |
| | yl)phenyl]methyl]carbamoyl)pyrrolidin- | | (d, J = 9.4 Hz, 1H), 4.49-4.38 (m, 2H), 4.35 |
| | 1-yl]-3,3-dimethyl-1-oxobutan-2- | | (s, 1H), 4.28-4.15 (m, 3H), 3.71-3.60 (m, |
| | yl]heptadecanediamide | | 2H), 3.55-3.46 (m, 2H), 2.93 (t, J = 7.5 Hz, |
| | | | 2H), 2.44 (s, 3H), 2.30-2.19 (m, 1H), 2.13- |
| | | | 2.08 (m, 3H), 2.05-1.96 (m, 1H), 1.96-1.85 |
| | | | (m, 1H), 1.55-1.38 (m, 4H), 1.29-1.18 (m, |
| | | | 22H), 0.97-0.85 (m, 9H). |
| 140 | N′-[[4-(2-[[3-amino-6-(2- | 932.5 | ¹H NMR (300 MHz, DMSO-d6) δ 8.95 (s, 1H), |
| | hydroxyphenyl)pyridazin-4- | | 8.56 (t, J = 6.0 Hz, 1H), 8.32-8.17 (m, 1H), |
| | yl]amino]ethyl)phenyl]methyl]- | | 7.98-7.75 (m, 2H), 7.50-7.34 (m, 4H), 7.33- |
| | N-[(2S)-1-[(2S,4R)-4-hydroxy- | | 7.24 (m, 2H), 7.23-7.13 (m, 3H), 7.00 (s, |
| | 2-([[4-(4-methyl-1,3-thiazol-5- | | 1H), 6.89-6.76 (m, 2H), 6.40-6.19 (m, 3H), |
| | yl)phenyl]methyl]carbamoyl)pyrrolidin- | | 5.14 (s, 1H), 4.56 (d, J = 9.2 Hz, 1H), 4.49- |
| | 1-yl]-3,3-dimethyl-1-oxobutan-2- | | 4.32 (m, 3H), 4.31-4.16 (m, 3H), 3.75-3.59 |
| | yl]decanediamide | | (m, 2H), 3.58-3.41 (m, 2H), 2.93 (t, J = 7.5 |
| | | | Hz, 2H), 2.43 (s, 3H), 2.21-2.05 (m, 4H), 2.03- |
| | | | 1.97 (m, 1H), 1.96-1.84 (m, 1H), 1.62- |
| | | | 1.40 (m, 4H), 1.23 (s, 8H), 0.92 (s, 9H). |
| 138 | N1-(4-(2-((3-amino-6-(2- | 933.3 | ¹H NMR (400 MHz, DMSO-d6) δ 14.39 (s, 1H), |
| | hydroxyphenyl)pyridazin-4- | | 8.98 (s, 1H), 8.57 (t, J = 6.1 Hz, 1H), 8.26 (t, |
| | yl)oxy)ethyl)benzyl)-N10-((S)-1- | | J = 5.9 Hz, 1H), 7.94 (d, J = 7.9 Hz, 1H), 7.85 (d, |
| | ((2S,4R)-4-hydroxy-2-((4-(4- | | J = 9.3 Hz, 1H), 7.59 (s, 1H), 7.45-7.30 (m, |
| | methylthiazol-5- | | 6H), 7.20 (dd, J = 12.1, 7.7 Hz, 3H), 6.87 (t, J = |
| | yl)benzyl)carbamoyl)pyrrolidin- | | 8.0 Hz, 2H), 6.51 (s, 2H), 5.13 (d, J = 3.5 Hz, |
| | 1-yl)-3,3-dimethyl-1-oxobutan-2- | | 1H), 4.54 (d, J = 9.3 Hz, 1H), 4.48-4.38 (m, |
| | yl)decanediamide | | 4H), 4.35 (s, 1H), 4.26-4.16 (m, 3H), 3.71- |
| | | | 3.60 (m, 2H), 3.12 (t, J = 6.9 Hz, 2H), 2.44 (s, |
| | | | 3H), 2.25 (dt, J = 14.7, 7.5 Hz, 1H), 2.10 (dd, |
| | | | J = 13.4, 5.9 Hz, 4H), 1.95-1.84 (m, 1H), 1.59- |
| | | | 1.38 (m, 4H), 1.23 (s, 8H), 0.93 (s, 9H). |
| 159 | N1-(4-(2-((3-amino-6-(2- | 1017.3 | ¹H NMR (300 MHz, DMSO-d6) δ 14.39 (s, 1H), |
| | hydroxyphenyl)pyridazin-4- | | 8.99 (s, 1H), 8.58 (d, J = 6.0 Hz, 1H), 7.94 (d, |
| | yl)oxy)ethyl)benzyl)-N15-((S)-1- | | J = 8.0 Hz, 1H), 7.84 (d, J = 9.5 Hz, 1H), 7.59 (s, |
| | ((2S,4R)-4-hydroxy-2-((4-(4- | | 1H), 7.38 (dd, J = 13.6, 6.1 Hz, 6H), 7.24 (t, J = |
| | methylthiazol-5- | | 7.4 Hz, 1H), 7.14 (t, J = 7.6 Hz, 2H), 6.88 (d, |
| | yl)benzyl)carbamoyl)pyrrolidin- | | J = 7.8 Hz, 2H), 6.52 (d, J = 6.8 Hz, 2H), 5.13 (d, |
| | 1-yl)-3,3-dimethyl-1-oxobutan-2- | | J = 3.5 Hz, 1H), 4.55 (d, J = 8.7 Hz, 2H), 4.44 |
| | yl)-N1-methylpentadecanediamide | | (d, J = 11.6 Hz, 5H), 4.35 (s, 1H), 4.21 (dd, J = |
| | | | 15.9, 5.4 Hz, 1H), 3.65 (s, 2H), 3.13 (s, 2H), |
| | | | 2.87 (s, 2H), 2.79 (s, 1H), 2.45 (s, 3H), 2.38- |
| | | | 2.20 (m, 3H), 2.15-1.97 (m, 2H), 1.92 (dd, J = |
| | | | 8.6, 4.4 Hz, 1H), 1.48 (s, 4H), 1.22 (d, J = 11.2 |
| | | | Hz, 18H), 0.93 (s, 9H). |
| 197 | N1-(4-(2-((3-amino-6-(2- | 1031.7 | ¹H NMR (300 MHz, DMSO-d6) δ 14.46 (s, 1H), |
| | hydroxyphenyl)pyridazin-4- | | 9.06 (s, 1H), 8.45 (d, J = 7.7 Hz, 1H), 8.01 (d, |
| | yl)oxy)ethyl)benzyl)-N15-((S)-1- | | J = 8.2 Hz, 1H), 7.86 (d, J = 9.2 Hz, 1H), 7.66 (s, |
| | ((2S,4R)-4-hydroxy-2-(((S)-1- | | 1H), 7.54-7.41 (m, 6H), 7.30 (ddd, J = 8.6, |
| | (4-(4-methylthiazol-5- | | 7.2, 1.5 Hz, 1H), 7.22 (t, J = 7.5 Hz, 2H), 6.94 |
| | yl)phenyl)ethyl)carbamoyl)pyrrolidin- | | (dd, J = 8.1, 6.9 Hz, 2H), 6.59 (s, 2H), 5.21(d, |
| | 1-yl)-3,3-dimethyl-1-oxobutan-2- | | J = 3.5 Hz, 1H), 5.00 (p, J = 6.9 Hz, 1H), 4.72- |
| | yl)-N1-methylpentadecanediamide | | 4.51 (m, 6H), 4.35 (s, 1H), 3.67 (s, 2H), 3.41 |
| | | | (s, 2H), 3.19 (t, J = 6.0 Hz, 2H), 2.86 (s, 1H), |
| | | | 2.57 (s, 3H), 2.52 (ddd, J = 22.3, 14.8, 7.5 Hz, |
| | | | 3H), 2.40-2.15 (m, 2H), 1.86 (ddd, J = 12.9, |
| | | | 8.5, 4.6 Hz, 1H), 1.53 (d, J = 7.0 Hz, 4H), 1.44 |
| | | | (d, J = 7.0 Hz, 3H), 1.29 (d, J = 10.6 Hz, 18H), |
| | | | 1.00 (s, 9H). |
| 214 | N1-(4-(2-((3-amino-6-(2- | 1031.45 | ¹H NMR (400 MHz, DMSO-d6) δ 14.38 (s, 1H), |
| | hydroxyphenyl)pyridazin-4- | | 8.98 (s, 1H), 8.37 (d, J = 7.8 Hz, 1H), 8.25 (t, |
| | yl)oxy)ethyl)benzyl)-N16-((S)-1- | | J = 5.9 Hz, 1H), 7.94 (d, J = 8.0, 1.6 Hz, 1H), |
| | ((2S,4R)-4-hydroxy-2-(((S)-1- | | 7.78 (d, J = 9.3 Hz, 1H), 7.59 (s, 1H), 7.47- |
| | (4-(4-methylthiazol-5- | | 7.40 (m, 2H), 7.40-7.30 (m, 4H), 7.27-7.14 |
| | yl)phenyl)ethyl)carbamoyl)pyrrolidin- | | (m, 3H), 6.91-6.82 (m, 2H), 6.50 (s, 2H), 5.09 |
| | 1-yl)-3,3-dimethyl-1-oxobutan-2- | | (d, J = 3.6 Hz, 1H), 4.97-4.85 (m, 1H), 4.51 |
| | yl)hexadecanediamide | | (d, J = 9.3 Hz, 1H), 4.47-4.37 (m, 3H), 4.27 |
| | | | (s, 1H), 4.22 (d, J = 5.9 Hz, 2H), 3.65-3.57 |
| | | | (m, 2H), 3.12 (t, J = 6.9 Hz, 2H), 2.45 (s, 3H), |
| | | | 2.30-2.18 (m, 1H), 2.15-2.04 (m, 3H), 2.07- |
| | | | 1.96 (m, 1H), 1.84-1.73 (m, 1H), 1.52- |
| | | | 1.47 (m, 4H), 1.37 (d, J = 7.0 Hz, 3H), 1.22 (s, |
| | | | 20H), 0.93 (s, 9H). |
| 215 | N1-(4-(2-((3-amino-6-(2- | 1045.45 | ¹H NMR (400 MHz, DMSO-d6) δ 14.38 (s, 1H), |
| | hydroxyphenyl)pyridazin-4- | | 8.98 (s, 1H), 8.37 (d, J = 7.8 Hz, 1H), 7.93 (d, |
| | yl)oxy)ethyl)benzyl)-N16-((S)-1- | | J = 8.1 Hz, 1H), 7.78 (d, J = 9.3 Hz, 1H), 7.59 (s, |
| | ((2S,4R)-4-hydroxy-2-(((S)-1- | | 1H), 7.47-7.30 (m, 6H), 7.27-7.18 (m, 1H), |
| | (4-(4-methylthiazol-5- | | 7.18-7.10 (m, 2H), 6.91-6.82 (m, 2H), 6.51 |
| | yl)phenyl)ethyl)carbamoyl)pyrrolidin- | | (s, 2H), 5.10 (d, J = 3.5 Hz, 1H), 4.98-4.85 |
| | 1-yl)-3,3-dimethyl-1-oxobutan-2- | | (m, 1H), 4.55-4.37 (m, 6H), 4.27 (s, 1H), 3.63- |
| | yl)-N1-methylhexadecanediamide | | 3.57 (m, 2H), 3.16-3.08 (m, 2H), 2.87 (s, |
| | | | 2H), 2.78 (s, 1H), 2.45 (s, 3H), 2.37-2.18 (m, |
| | | | 3H), 2.15-1.94 (m, 2H), 1.84-1.73 (m, 1H), |
| | | | 1.53-1.43 (m, 4H), 1.37 (d, J = 7.0 Hz, 3H), |
| | | | 1.25-1.18 (m, 20H), 0.93 (s, 9H). |
| 223 | N1-(4-(2-((3-amino-6-(2- | 1031.35 | ¹H NMR (300 MHz, DMSO-d6) δ 14.39 (br s, |
| | hydroxyphenyl)pyridazin-4- | | 1H), 8.99 (s, 1H), 8.57 (t, J = 6.1 Hz, 1H), 7.94 |
| | yl)oxy)ethyl)benzyl)-N16-((S)-1- | | (d, J = 8.0 Hz, 1H), 7.84 (d, J = 9.4 Hz, 1H), |
| | ((2S,4R)-4-hydroxy-2-((4-(4- | | 7.59 (s, 1H), 7.46-7.32 (m, 6H), 7.27-7.19 |
| | methylthiazol-5- | | (m, 1H), 7.19-7.11 (m, 2H), 6.91-6.83 (m, |
| | yl)benzyl)carbamoyl)pyrrolidin- | | 2H), 6.52 (s, 2H), 5.13 (s, 1H), 4.59-4.30 (m, |
| | 1-yl)-3,3-dimethyl-1-oxobutan-2- | | 8H), 4.28-4.15 (m, 1H), 3.66 (d, J = 4.3 Hz, |
| | yl)-N1-methylhexadecanediamide | | 2H), 3.13 (t, J = 6.3 Hz, 2H), 2.87 (s, 3H), 2.45 |
| | | | (s, 3H), 2.38-2.18 (m, 3H), 2.16-1.98 (m, |
| | | | 2H), 1.96-1.84 (m, 1H), 1.49 (s, 4H), 1.22 (d, |
| | | | J = 8.9 Hz, 20H), 0.94 (s, 9H). |
| 198 | N1-(4-(2-((3-amino-6-(2- | 961.30 | ¹H NMR (400 MHz, DMSO-d6) δ 8.96 (s, 1H), |
| | hydroxyphenyl)pyridazin-4- | | 8.41 (d, J = 7.7 Hz, 1H), 8.34 (t, J = 6.0 Hz, |
| | yl)oxy)ethyl)benzyl)-N11-((S)-1- | | 1H), 7.91 (dd, J = 8.4, 1.6 Hz, 1H), 7.80 (d, J = |
| | ((2S,4R)-4-hydroxy-2-(((S)-1- | | 9.3 Hz, 1H), 7.55 (s, 1H), 7.46-7.31 (m, 6H), |
| | (4-(4-methylthiazol-5- | | 7.29-7.16 (m, 3H), 6.95-6.87 (m, 2H), 4.94- |
| | yl)phenyl)ethyl)carbamoyl)pyrrolidin- | | 4.86 (m, 1H), 4.51 (d, J = 9.3 Hz, 1H), 4.47- |
| | 1-yl)-3,3-dimethyl-1-oxobutan-2- | | 4.40 (m, 3H), 4.29 (d, J = 4.5 Hz, 1H), 4.23 (d, |
| | yl)undecanediamide | | J = 5.9 Hz, 2H), 3.61 (d, J = 3.1 Hz, 2H), 3.13 |
| | | | (t, J = 6.6 Hz, 2H), 2.45 (s, 3H), 2.29-2.18 (m, |
| | | | 1H), 2.16-2.00 (m, 4H), 1.85-1.75 (m, 1H), |
| | | | 1.58-1.42 (m, 4H), 1.38 (d, J = 7.0 Hz, 3H), |
| | | | 1.23 (s, 10H), 0.93 (s, 9H). |
| 227 | N1-(4-(2-((3-amino-6-(2- | 530.70 | ¹H NMR (400 MHz, DMSO-d6) δ 8.99 (s, 1H), |
| | hydroxyphenyl)pyridazin-4- | (M/2 + H) | 8.38 (d, J = 7.8 Hz, 1H), 7.78 (d, J = 9.3 Hz, |
| | yl)oxy)ethyl)benzyl)-N17-((S)-1- | | 1H), 7.66 (s, 1H), 7.62 (d, J = 7.6 Hz, 1H), 7.59- |
| | ((2S,4R)-4-hydroxy-2-(((S)-1- | | 7.49 (m, 1H), 7.43 (d, J = 8.3 Hz, 2H), 7.41- |
| | (4-(4-methylthiazol-5- | | 7.29 (m, 5H), 7.15 (q, J = 9.3, 8.3 Hz, 2H), 7.04 |
| | yl)phenyl)ethyl)carbamoyl)pyrrolidin- | | (d, J = 8.6 Hz, 1H), 6.96 (t, J = 7.6 Hz, 1H), |
| | 1-yl)-3,3-dimethyl-1-oxobutan-2- | | 4.92 (q, J = 7.0 Hz, 1H), 4.61-4.48 (m, 4H), |
| | yl)-N1-methylheptadecanediamide | | 4.46 (s, 1H), 4.42 (t, J = 8.0 Hz, 1H), 4.28 (d, |
| | | | J = 4.8 Hz, 1H), 3.64-3.55 (m, 2H), 3.51 (s, |
| | | | 1H), 3.14 (t, J = 6.7 Hz, 2H), 2.87 (s, 2H), 2.78 |
| | | | (d, J = 4.3 Hz, 1H), 2.45 (s, 3H), 2.34-2.21 |
| | | | (m, 3H), 2.12-1.98 (m, 2H), 1.83-1.75 (m, |
| | | | 1H), 1.55-1.42 (m, 4H), 1.37 (d, J = 7.0 Hz, |
| | | | 3H), 1.31-1.13 (m, 22H), 0.93 (s, 9H). |
| 228 | N1-(4-(2-((3-amino-6-(2- | 1045.60 | ¹H NMR (400 MHz, DMSO-d6) δ 14.38 (s, 1H), |
| | hydroxyphenyl)pyridazin-4- | | 8.98 (s, 1H), 8.56 (t, J = 6.1 Hz, 1H), 7.93 (d, |
| | yl)oxy)ethyl)benzyl)-N17-((S)-1- | | J = 7.9 Hz, 1H), 7.84 (d, J = 9.3 Hz, 1H), 7.59 (s, |
| | ((2S,4R)-4-hydroxy-2-((4-(4- | | 1H), 7.45-7.33 (m, 6H), 7.23 (td, J = 8.3, 7.8, |
| | methylthiazol-5- | | 1.6 Hz, 1H), 7.14 (t, J = 8.8 Hz, 2H), 6.86 (t, |
| | yl)benzyl)carbamoyl)pyrrolidin- | | J = 8.2 Hz, 2H), 6.52 (d, J = 9.0 Hz, 2H), 5.12 (d, |
| | 1-yl)-3,3-dimethyl-1-oxobutan-2- | | J = 3.6 Hz, 1H), 4.54 (d, J = 9.4 Hz, 2H), 4.49- |
| | yl)-N1-methylheptadecanediamide | | 4.38 (m, 5H), 4.35 (s, 1H), 4.21 (dd, J = 15.9, |
| | | | 5.5 Hz, 1H), 3.65 (d, J = 6.4 Hz, 2H), 3.12 (t, |
| | | | J = 6.6 Hz, 2H), 2.86 (s, 2H), 2.78 (s, 1H), 2.44 |
| | | | (s, 3H), 2.33-2.23 (m, 3H), 2.12-2.00 (m, |
| | | | 2H), 1.93-1.86 (m, 1H), 1.55-1.41 (m, 4H), |
| | | | 1.31-1.15 (m, 22H), 0.93 (s, 9H). |
| 255 | N1-(4-(2-((3-amino-6-(2- | 1029 | ¹H NMR (300 MHz, DMSO-d6) δ 14.34 (s, 1H), |
| | hydroxyphenyl)pyridazin-4- | | 8.99 (s, 1H), 8.38 (d, J = 7.7 Hz, 1H), 8.20 (t, |
| | yl)(methyl)amino)ethyl)benzyl)- | | J = 5.9 Hz, 1H), 7.90 (dd, J = 8.4, 1.6 Hz, 1H), |
| | N15-((S)-1-((2S,4R)-4-hydroxy- | | 7.79 (d, J = 9.2 Hz, 1H), 7.50-7.09 (m, 10H), |
| | 2-(((S)-1-(4-(4-methylthiazol-5- | | 6.95-6.83 (m, 2H), 6.10 (s, 2H), 5.11 (d, J = |
| | yl)phenyl)ethyl)carbamoyl)pyrrolidin- | | 3.5 Hz, 1H), 4.92 (p, J = 7.0 Hz, 1H), 4.52 (d, |
| | 1-yl)-3,3-dimethyl-1-oxobutan-2- | | J = 9.3 Hz, 1H), 4.42 (t, J = 8.0 Hz, 1H), 4.28 (s, |
| | yl)pentadecanediamide | | 1H), 4.18 (d, J = 5.9 Hz, 2H), 3.60 (s, 2H), 3.37 |
| | | | (d, J = 3.0 Hz, 1H), 2.94 (s, 3H), 2.79 (t, J = 7.8 |
| | | | Hz, 2H), 2.46 (s, 3H), 2.22 (t, J = 7.4 Hz, 1H), |
| | | | 2.16-1.94 (m, 5H), 1.79 (m, J = 12.9, 8.6, 4.6 |
| | | | Hz, 1H), 1.55-1.43 (m, 4H), 1.38 (d, J = 7.0 |
| | | | Hz, 3H), 1.23 (s, 19H), 0.94 (s, 9H). |
| 247 | N1-(4-(2-((3-amino-6-(2- | 975.4 | ¹H NMR (400 MHz, DMSO-d6) δ 14.38 (s, 1H), |
| | hydroxyphenyl)pyridazin-4- | | 8.98 (s, 1H), 8.37 (d, J = 7.8 Hz, 1H), 8.25 (t, |
| | yl)oxy)ethyl)benzyl)-N12-((S)-1- | | J = 5.9 Hz, 1H), 7.94 (dd, J = 8.1, 1.6 Hz, 1H), |
| | ((2S,4R)-4-hydroxy-2-(((S)-1- | | 7.77 (d, J = 9.3 Hz, 1H), 7.59 (s, 1H), 7.48- |
| | (4-(4-methylthiazol-5- | | 7.28 (m, 6H), 7.28-7.13 (m, 3H), 6.91-6.80 |
| | yl)phenyl)ethyl)carbamoyl)pyrrolidin- | | (m, 2H), 6.50 (s, 2H), 5.09 (d, J = 3.4 Hz, 1H), |
| | 1-yl)-3,3-dimethyl-1-oxobutan-2- | | 4.97-4.82 (m, 1H), 4.51 (d, J = 9.3 Hz, 1H), |
| | yl)dodecanediamide | | 4.47-4.38 (m, 3H), 4.27 (s, 1H), 4.22 (d, J = |
| | | | 5.9 Hz, 2H), 3.65-3.55 (m, 2H), 3.12 (t, J = |
| | | | 6.8 Hz, 2H), 2.45 (s, 3H), 2.24 (m, 1H), 2.10 |
| | | | (m, 3H), 2.05-1.96 (m, 1H), 1.87-1.70 (m, |
| | | | 1H), 1.56-1.41 (m, 4H), 1.37 (d, J = 7.0 Hz, |
| | | | 3H), 1.23 (s, 12H), 0.93 (s, 9H). |
| 257 | N1-(4-(2-((3-amino-6-(2- | 947.30 | ¹H NMR (300 MHz, DMSO-d6) δ 14.37 (br s, |
| | hydroxyphenyl)pyridazin-4- | | 1H), 8.99 (s, 1H), 8.39 (d, J = 7.8 Hz, 1H), 8.31- |
| | yl)oxy)ethyl)benzyl)-N10-((S)-1- | | 8.23 (m, 1H), 8.00-7.90 (m, 1H), 7.79 (d, J = |
| | ((2S,4R)-4-hydroxy-2-(((S)-1- | | 9.2 Hz, 1H), 7.60 (s, 1H), 7.47-7.31 (m, 6H), |
| | (4-(4-methylthiazol-5- | | 7.27-7.14 (m, 3H), 6.94-6.80 (m, 2H), 6.51 (s, |
| | yl)phenyl)ethyl)carbamoyl)pyrrolidin- | | 2H), 5.11 (br s, 1H), 4.96-4.87 (m, 1H), 4.57- |
| | 1-yl)-3,3-dimethyl-1-oxobutan- | | 4.36 (m, 4H), 4.32-4.19 (m, 3H), 3.60 (s, 2H), |
| | 2-yl)decanediamide | | 3.12 (t, J = 6.8 Hz, 2H), 2.46 (s, 3H), 2.33-2.16 |
| | | | (m, 1H), 2.16-2.07 (m, 3H), 2.04-1.92 (m, |
| | | | 1H), 1.88-1.69 (m, 1H), 1.58-1.42 (m, 4H), |
| | | | 1.37 (d, J = 7.0 Hz, 3H), 1.24 (s, 8H), 0.93 (s, |
| | | | 9H). |
| 248 | N1-(4-(2-((3-amino-6-(2- | 1003.54 | ¹H NMR (300 MHz, DMSO-d6) δ 14.4 (s, 1H), |
| | hydroxyphenyl)pyridazin-4- | | 8.99 (s, 1H), 8.38 (d, J = 7.9 Hz, 1H), 8.25 (d, |
| | yl)oxy)ethyl)benzyl)-N14-((S)-1- | | J = 5.8 Hz, 1H), 8.14 (s, 1H), 7.94 (d, J = 7.6 Hz, |
| | ((2S,4R)-4-hydroxy-2-(((S)-1- | | 1H), 7.78 (d, J = 9.1 Hz, 1H), 7.60 (s, 1H), 7.39 |
| | (4-(4-methylthiazol-5- | | (dt, J = 17.1, 8.2 Hz, 6H), 7.21 (t, J = 8.3 Hz, |
| | yl)phenyl)ethyl)carbamoyl)pyrrolidin- | | 3H), 6.87 (t, J = 7.7 Hz, 2H), 6.52 (s, 2H), 5.10 |
| | 1-yl)-3,3-dimethyl-1-oxobutan-2- | | (d, J = 3.5 Hz, 1H), 4.97-4.87 (m, 1H), 4.52 |
| | yl)tetradecanediamide | | (d, J = 9.4 Hz, 1H), 4.43 (d, J = 4.3 Hz, 3H), |
| | | | 4.28 (s, 1H), 4.23 (d, J = 5.9 Hz, 2H), 3.60 (s, |
| | | | 2H), 3.12 (t, J = 6.8 Hz, 2H), 2.46 (s, 3H), 2.24 |
| | | | (d, J = 7.8 Hz, 1H), 2.11 (t, J = 7.2 Hz, 3H), |
| | | | 2.01 (s, 2H), 1.79 (s, 1H), 1.48 (s, 4H), 1.38 (d, |
| | | | J = 7.0 Hz, 3H), 1.23 (s, 16H), 0.94 (s, 9H). |
| 265 | (2S,4R)-1-((S)-2-(3-(4-(5-((4-(2- | 1003.55 | ¹H NMR (400 MHz, DMSO-d₆) δ 14.38 (s, 1H), |
| | ((3-amino-6-(2- | | 8.98 (s, 1H), 8.40 (dd, J = 11.2, 8.6 Hz, 2H), |
| | hydroxyphenyl)pyridazin-4- | | 8.26 (t, J = 5.9 Hz, 1H), 7.94 (dd, J = 8.0, 1.6 |
| | yl)oxy)ethyl)benzyl)amino)-5- | | Hz, 1H), 7.59 (s, 1H), 7.47-7.40 (m, 2H), 7.40- |
| | oxopentyl)piperazin-1- | | 7.31 (m, 4H), 7.27-7.16 (m, 3H), 6.86 (t, J = |
| | yl)propanamido)-3,3- | | 7.8 Hz, 2H), 6.50 (s, 2H), 5.10 (d, J = 3.5 Hz, |
| | dimethylbutanoyl)-4-hydroxy-N- | | 1H), 4.90 (p, J = 7.1 Hz, 1H), 4.51 (d, J = 9.4 |
| | ((S)-1-(4-(4-methylthiazol-5- | | Hz, 1H), 4.42 (td, J = 7.9, 7.4, 3.7 Hz, 3H), |
| | yl)phenyl)ethyl)pyrrolidine-2- | | 4.31-4.18 (m, 3H), 3.65-3.54 (m, 2H), 3.12 |
| | carboxamide | | (t, J = 6.8 Hz, 2H), 2.47-2.43 (m, 4H), 2.42- |
| | | | 2.33 (m, 4H), 2.28-2.19 (m, 4H), 2.12 (t, J = |
| | | | 7.3 Hz, 2H), 2.06-1.96 (m, 1H), 1.78 (ddd, J = |
| | | | 12.9, 8.4, 4.6 Hz, 1H), 1.49 (dt, J = 15.1, 7.3 |
| | | | Hz, 2H), 1.37 (d, J = 7.0 Hz, 4H), 0.94 (s, 9H). |

### Preparation of N-(4-(2-((3-amino-6-(2-hydroxyphenyl)pyridazin-4-yl)oxy)ethyl)benzyl)-5-((2-(2,6-dioxopiperidin-3-yl)-3-oxoisoindolin-5-yl)oxy)pentanamide (Compound 128) and N-(4-(2-((3-amino-6-(2-hydroxyphenyl)pyridazin-4-yl)oxy)ethyl)benzyl)-5-((2-(2,6-dioxopiperidin-3-yl)-1-oxoisoindolin-5-yl)oxy)pentanamide (Compound 129)

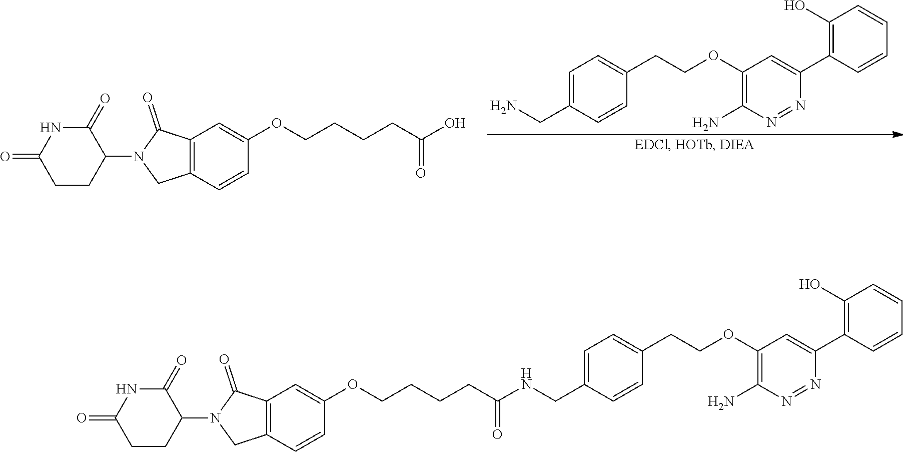

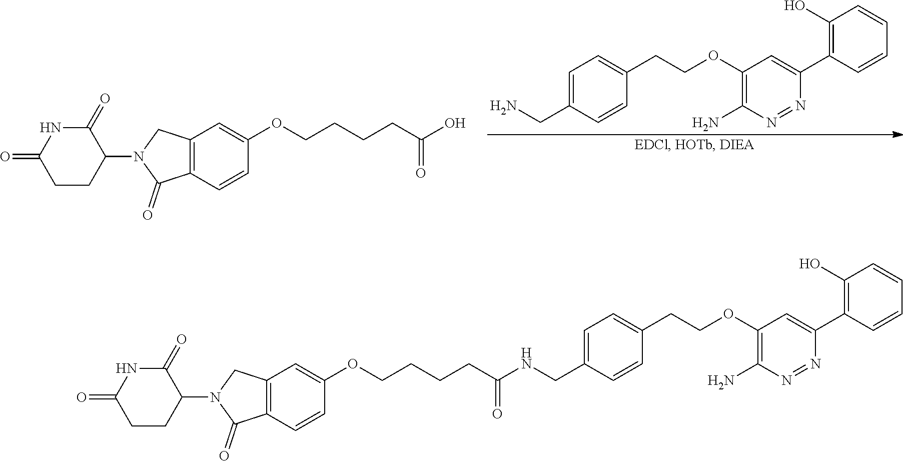

### Step 1: Preparation of N-(4-(2-((3-amino-6-(2-hydroxyphenyl)pyridazin-4-yl)oxy)ethyl)benzyl)-5-((2-(2,6-dioxopiperidin-3-yl)-3-oxoisoindolin-5-yl)oxy)pentanamide (Compound 128) and N-(4-(2-((3-amino-6-(2-hydroxyphenyl)pyridazin-4-yl)oxy)ethyl) benzyl)-5-((2-(2,6-dioxopiperidin-3-yl)-1-oxoisoindolin-5-yl)oxy)pentanamide (Compound 129)

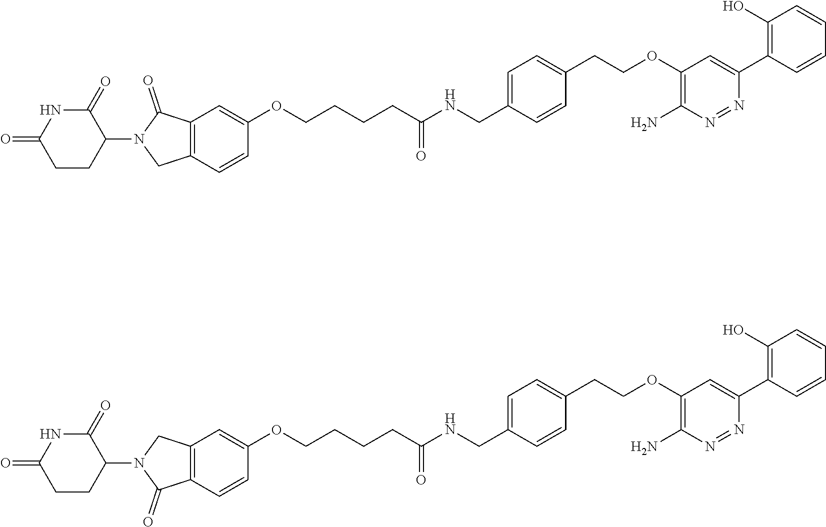 [SMILES: Nc1nnc(-c2ccccc2O)cc1OCCc1ccc(CNC(=O)CCCCOc2ccc3c(c2)C(=O)N(C2CCC(=O)NC2=O)C3)cc1 ; Nc1nnc(-c2ccccc2O)cc1OCCc1ccc(CNC(=O)CCCCOc2ccc3c(c2)CN(C2CCC(=O)NC2=O)C3=O)cc1]

To a solution of 5-((2-(2,6-dioxopiperidin-3-yl)-3-oxoisoindolin-5-yl)oxy)pentanoic acid (20.0 mg, 0.055 mmol, 1.00 equiv) in DMF (1.00 mL) was added HOBT (15.0 mg, 0.111 mmol, 2.00 equiv) and EDCI (21.2 mg, 0.111 mmol, 2.00 equiv). After stirring for 0.5 h at room temperature, 2-(6-amino-5-[2-[4-(aminomethyl)phenyl]ethoxy]pyridazin-3-yl)phenol (18.6 mg, 0.055 mmol, 1.00 equiv) and DIEA (21.5 mg, 0.166 mmol, 3.00 equiv) were added. The resulting mixture was stirred for 2 hrs at room temperature. The reaction mixture was filtered and concentrated in vacuo. The residue was purified by Prep-HPLC with follow conditions: Column: Xcelect CSH F-pheny OBD Column, 19*250 mm, 5 um; Mobile Phase A:Water (0.05% FA), Mobile Phase B:ACN; Flow rate: 25 mL/min; Gradient: 24% B to 40% B in 10 min 254/220 nm; RT: 8.60 to afford N-(4-(2-((3-amino-6-(2-hydroxyphenyl)pyridazin-4-yl)oxy)ethyl)benzyl)-5-((2-(2,6-dioxopiperidin-3-yl)-3-oxoisoindolin-5-yl)oxy)pentanamide (Compound 128, 10.4 mg, 26.2%) as a white solid. ¹H NMR (300 MHz, DMSO-d₆) δ 14.37 (s, 1H), 10.98 (s, 1H), 8.32 (t, J=6.3 Hz, 1H), 7.95 (d, J=7.9 Hz, 1H), 7.60 (s, 1H), 7.49 (d, J=8.3 Hz, 1H), 7.35 (d, J=7.9 Hz, 2H), 7.20 (m, 5H), 6.87 (m, 2H), 6.51 (s, 2H), 5.11 (dd, J=13.3, 5.1 Hz, 1H), 4.49-4.37 (m, 2H), 4.34 (s, 1H), 4.30-4.18 (m, 3H), 4.11-4.01 (m, 2H), 3.12 (t, J=6.8 Hz, 2H), 3.01-2.82 (m, 1H), 2.66-2.54 (m, 1H), 2.44-2.30 (m, 1H), 2.22 (t, J=6.7 Hz, 2H), 2.05-1.93 (m, 1H), 1.84-1.61 (m, 4H). LCMS (ESI) m/z: [M+H]⁺=679.35.

To a solution of 5-((2-(2,6-dioxopiperidin-3-yl)-1-oxoisoindolin-5-yl)oxy)pentanoic acid (15.0 mg, 0.042 mmol, 1.00 equiv) in DMF (1.00 mL) was added HOBT (11.2 mg, 0.083 mmol, 2.00 equiv) and EDCI (15.9 mg, 0.083 mmol, 2.00 equiv). After stirring for 0.5 h at room temperature, 2-(6-amino-5-[2-[4-(aminomethyl)phenyl]ethoxy]pyridazin-3-yl)phenol (14.0 mg, 0.042 mmol, 1.00 equiv) and DIEA (16.1 mg, 0.125 mmol, 3.00 equiv) were added. The resulting mixture was stirred for 2 hrs at room temperature. The reaction mixture was filtered and concentrated in vacuo. The residue was purified by Prep-HPLC with follow conditions: Column: Column: Gemini-NX C₁₈ AXAI Packed, 21.2*150 mm Sum; Mobile Phase A:Water (0.05% FA), Mobile Phase B:ACN; Flow rate: 25 mL/min; Gradient: 35% B to 53% B in 10 min; 254/220 nm; RT: 9.85 to afford compound N-(4-(2-((3-amino-6-(2-hydroxyphenyl)pyridazin-4-yl)oxy)ethyl)benzyl)-5-((2-(2,6-dioxopiperidin-3-yl)-1-oxoisoindolin-5-yl)oxy)pentanamide (Compound 129, 10.3 mg, 35.5%) as a white solid. ¹H NMR (300 MHz, DMSO-d₆) δ 10.97 (s, 1H), 8.35 (t, J=5.9 Hz, 1H), 7.69-7.58 (m, 3H), 7.48 (s, 1H), 7.43-7.29 (m, 4H), 7.24-7.12 (m, 3H), 7.04 (dd, J=8.3, 2.8 Hz, 2H), 6.96 (t, J=7.5 Hz, 1H), 5.08 (dd, J=13.2, 5.0 Hz, 1H), 4.52 (t, J=6.8 Hz, 2H), 4.39 (d, J=17.2 Hz, 1H), 4.32-4.20 (m, 3H), 4.12-4.02 (m, 2H), 3.14 (t, J=6.8 Hz, 2H), 3.00-2.82 (m, 1H), 2.66-2.55 (m, 1H), 2.43-2.32 (m, 1H), 2.22 (t, J=6.7 Hz, 2H), 2.03-1.91 (m, 1H), 1.81-1.60 (m, 4H). LCMS (ESI) m/z: [M+H]⁺=679.40.

The following compounds in Table E6 were prepared using procedures similar to those used for the preparation of compound 129.

TABLE E6

| | | LCMS | |
|---|---|---|---|
| | | (ESI) | |
| No. | Name | m/z | ¹H NMR |
| 130 | N-(4-(2-((3-amino-6-(2- | 679.4 | ¹H NMR (400 MHz, DMSO-d6) δ 10.97 (s, 1H), |
| | hydroxyphenyl)pyridazin-4- | | 8.34 (s, 1H), 7.66 (s, 2H), 7.59 (s, 1H), 7.47 (t, |
| | yl)oxy)ethyl)benzyl)-5-((2-(2,6- | | J = 7.8 Hz, 1H), 7.39 (t, J = 7.8 Hz, 1H), 7.35- |
| | dioxopiperidin-3-yl)-1- | | 7.28 (m, 3H), 7.25-7.15 (m, 3H), 7.10-7.02 |
| | oxoisoindolin-4- | | (m, 1H), 6.97 (t, J = 7.5 Hz, 1H), 5.14-5.06 |
| | yl)oxy)pentanamide | | (m, 1H), 4.53 (t, J = 6.9 Hz, 2H), 4.41-4.33 |
| | | | (m, 1H), 4.27-4.17 (m, 3H), 4.12 (t, J = 5.9 |
| | | | Hz, 2H), 3.13 (t, J = 6.8 Hz, 2H), 2.98-2.83 |
| | | | (m, 1H), 2.70-2.53 (m, 1H), 2.47-2.36 (m, |
| | | | 1H), 2.21 (t, J = 6.9 Hz, 2H), 2.02-1.93 (m, |
| | | | 1H), 1.79-1.64 (m, 4H). |
| 137 | N-(4-(2-((3-amino-6-(2- | 678.4 | ¹H NMR (300 MHz, DMSO-d6) δ 14.38 (s, 1H), |
| | hydroxyphenyl)pyridazin-4- | | 10.93 (s, 1H), 8.34-8.23 (m, 1H), 8.00-7.90 |
| | yl)oxy)ethyl)benzyl)-5-((2-(2,6- | | (m, 1H), 7.60 (s, 1H), 7.36 (dd, J = 13.5, 8.2 |
| | dioxopiperidin-3-yl)-1- | | Hz, 3H), 7.29-7.14 (m, 3H), 6.94-6.81 (m, |
| | oxoisoindolin-5- | | 2H), 6.69-6.59 (m, 2H), 6.51 (s, 2H), 6.37 (m, |
| | yl)amino)pentanamide | | J = 5.6 Hz, 1H), 5.02 (dd, J = 13.3, 5.1 Hz, 1H), |
| | | | 4.43 (t, J = 6.8 Hz, 2H), 4.32-4.08 (m, 4H), |
| | | | 3.17-3.04 (m, 4H), 2.95-2.81 (m, 1H), 2.76- |
| | | | 2.68 (m, 1H), 2.39-2.26 (m, 1H), 2.18 (t, J = |
| | | | 6.9 Hz, 2H), 2.00-1.87 (m, 1H), 1.70-1.46 |
| | | | (m, 4H). |

### Preparation of (2S,4R)-1-((S)-2-(3-(6-(2-((4-(2-((3-amino-6-(2-hydroxyphenyl)pyridazin-4-yl)oxy)ethyl)benzyl)amino)-2-oxoethyl)-2,6-diazaspiro[3.3]heptan-2-yl)propanamido)-3,3-dimethylbutanoyl)-4-hydroxy-N-(4-(4-methylthiazol-5-yl)benzyl)pyrrolidine-2-carboxamide (Compound 196)

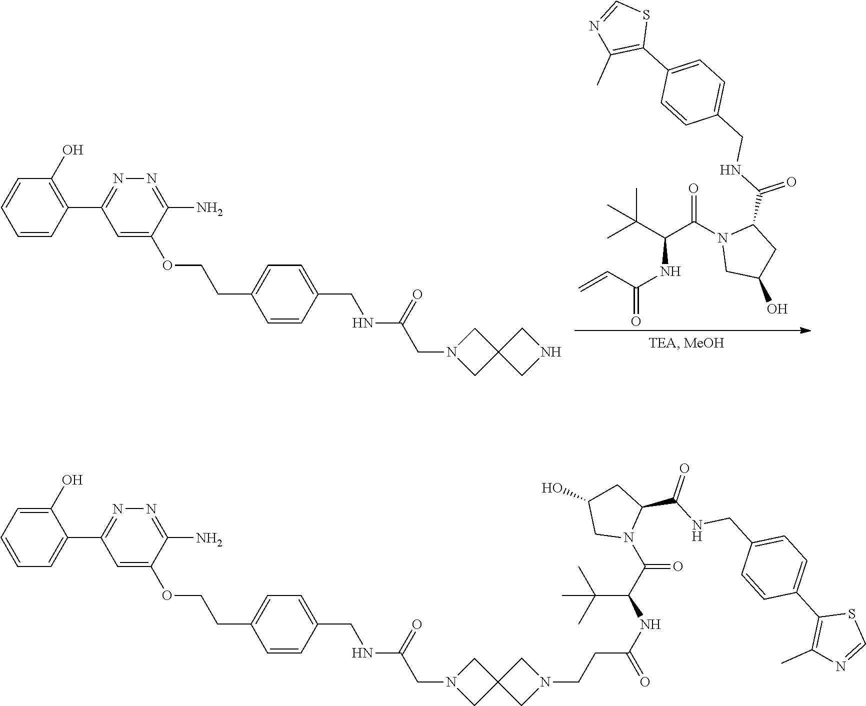 [SMILES: Nc1nnc(-c2ccccc2O)cc1OCCc1ccc(CNC(=O)CN2CC3(CNC3)C2)cc1 ; C=CC(=O)N[C@H](C(=O)N1C[C@H](O)C[C@H]1C(=O)NCc1ccc(-c2scnc2C)cc1)C(C)(C)C ; Cc1ncsc1-c1ccc(CNC(=O)[C@@H]2C[C@@H](O)CN2C(=O)[C@@H](NC(=O)CCN2CC3(C2)CN(CC(=O)NCc2ccc(CCOc4cc(-c5ccccc5O)nnc4N)cc2)C3)C(C)(C)C)cc1]

### Step 1: Preparation of (2S,4R)-1-((S)-2-(3-(6-(2-((4-(2-((3-amino-6-(2-hydroxyphenyl)pyridazin-4-yl)oxy)ethyl)benzyl)amino)-2-oxoethyl)-2,6-diazaspiro[3.3]heptan-2-yl)propanamido)-3,3-dimethylbutanoyl)-4-hydroxy-N-(4-(4-methylthiazol-5-yl)benzyl)pyrrolidine-2-carboxamide (Compound 196)

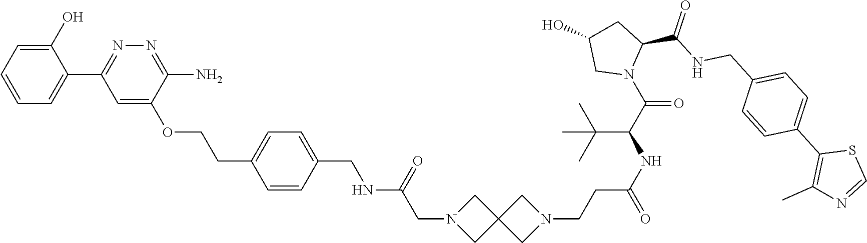 [SMILES: Cc1ncsc1-c1ccc(CNC(=O)[C@@H]2C[C@@H](O)CN2C(=O)[C@@H](NC(=O)CCN2CC3(C2)CN(CC(=O)NCc2ccc(CCOc4cc(-c5ccccc5O)nnc4N)cc2)C3)C(C)(C)C)cc1]

To a solution of N-(4-(2-((3-amino-6-(2-hydroxyphenyl)pyridazin-4-yl)oxy)ethyl)benzyl)-2-(2,6-diazaspiro[3.3]heptan-2-yl)acetamide (20.00 mg, 0.042 mmol, 1.00 equiv) and (2S,4R)-1-((S)-2-acrylamido-3,3-dimethylbutanoyl)-4-hydroxy-N-(4-(4-methylthiazol-5-yl)benzyl)pyrrolidine-2-carboxamide (20.42 mg, 0.042 mmol, 1.00 equiv) in MeOH (3.00 mL) was added TEA (0.20 mL) dropwise at 60° C. After stirring for 10 h the resulting mixture was concentrated under reduced pressure. The residue was purified by reverse phase flash with the following conditions (Column: XBridge Shield RP18 OBD Column, 5 um, 19*150 mm; Mobile Phase A:Water (10 mM NH₄HCO₃), Mobile Phase B:ACN; Flow rate: 25 mL/min; Gradient: 32 B to 47 B in 10 min, 47 B to B in min, B to B in min, B to B in min, B to B in min; 254/220 nm/) to afford the title compound (11.5 mg, 27.97%) as a yellow solid. ¹H NMR (400 MHz, DMSO-d⁶, with a drop of D₂O) δ 9.02-8.88 (m, 1H), 7.96-7.88 (m, 1H), 7.60-7.50 (m, 1H), 7.47-7.30 (m, 4H), 7.37-7.21 (m, 2H), 7.23 (d, J=1.5 Hz, 1H), 7.21-7.11 (m, 2H), 6.96-6.82 (m, 2H), 4.53-4.28 (m, 6H), 4.27-4.16 (m, 3H), 3.73-3.63 (m, 2H), 3.63-3.59 (m, 2H), 3.34-3.21 (m, 4H), 3.23-3.10 (m, 6H), 3.10-2.92 (m, 2H), 2.75-2.52 (m, 3H), 2.49-2.28 (m, 2H), 2.23-2.00 (m, 1H), 1.99-1.80 (3, 1H), 0.93 (s, 9H). LCMS (ESI) m/z: [M+H]⁺=959.

The following compounds in Table E7 were prepared using procedures similar to those used for the preparation of compound 196.

TABLE E7

| | | LCMS | |
|---|---|---|---|
| | | (ESI) | |
| No. | Name | m/z | ¹H NMR |
| 179 | (2S,4R)-1-[(2S)-2-(3-[4-[([[4-(2- | 947.5 | ¹H NMR (400 MHz, DMSO-d⁶) δ 14.38 (s, 1H), |
| | [[3-amino-6-(2- | | 8.96 (s, 1H), 8.58 (t, J = 6.1 Hz, 1H), 8.42 (d, |
| | hydroxyphenyl)pyridazin-4- | | J = 9.4 Hz, 1H), 8.19 (s, 1H), 7.94 (dd, J = 8.1, |
| | yl]oxy]ethyl)phenyl]meth- | | 1.6 Hz, 1H), 7.59 (s, 1H), 7.48-7.31 (m, 6H), |
| | yl]carbamoyl)methyl]piperazin- | | 7.27-7.16 (m, 3H), 6.86 (t, J = 7.8 Hz, 2H), |
| | 1-yl]propanamido)-3,3- | | 6.50 (s, 2H), 5.13 (d, J = 3.5 Hz, 1H), 4.54 (d, |
| | dimethylbutanoyl]-4-hydroxy-N- | | J = 9.4 Hz, 1H), 4.47-4.38 (m, 4H), 4.35 (s, |
| | [[4-(4-methyl-1,3-thiazol-5- | | 1H), 4.29-4.18 (m, 3H), 3.69-3.59 (m, 2H), |
| | yl)phenyl]methyl]pyrrolidine-2- | | 3.31 (s, 3H), 3.12 (t, J = 6.8 Hz, 2H), 2.93 (s, |
| | carboxamide | | 2H), 2.51 (s, 2H), 2.44 (s, 3H), 2.40 (s, 4H), |
| | | | 2.30 (s, 2H), 2.35-2.21 (m, 1H), 2.04 (dd, J = |
| | | | 19.9, 8.1 Hz, 1H), 1.90 (ddd, J = 12.8, 8.6, 4.5 |
| | | | Hz, 1H), 0.94 (s, 9H). |
| 180 | N-(4-(2-((3-amino-6-(2- | 932.15 | ¹H NMR (400 MHz, DMSO-d⁶) δ 14.38 (s, 1H), |
| | hydroxyphenyl)pyridazin-4- | | 8.96 (s, 1H), 8.57 (t, J = 6.1 Hz, 1H), 8.42 (d, |
| | yl)oxy)ethyl)benzyl)-1-(3-(((S)- | | J = 9.4 Hz, 1H), 8.24 (t, J = 6.0 Hz, 1H), 7.94 (d, |
| | 1-((2S,4R)-4-hydroxy-2-((4-(4- | | J = 7.9 Hz, 1H), 7.59 (s, 1H), 7.47-7.37 (m, |
| | methylthiazol-5- | | 4H), 7.33 (d, J = 7.8 Hz, 2H), 7.27-7.20 (m, |
| | yl)benzyl)carbamoyl)pyrrolidin- | | 1H), 7.16 (d, J = 7.8 Hz, 2H), 6.86 (t, J = 8.1 |
| | 1-yl)-3,3-dimethyl-1-oxobutan- | | Hz, 2H), 6.50 (s, 2H), 5.12 (d, J = 3.3 Hz, 1H), |
| | 2-yl)amino)-3- | | 4.53 (d, J = 9.4 Hz, 1H), 4.40 (dt, J = 22.3, 9.2 |
| | oxopropyl)piperidine-4- | | Hz, 5H), 4.30-4.09 (m, 3H), 3.76-3.57 (m, |
| | carboxamide | | 2H), 3.11 (t, J = 6.8 Hz, 2H), 3.04-2.81 (m, |
| | | | 2H), 2.67 (s, 1H), 2.43 (s, 4H), 2.29 (dt, J = |
| | | | 14.2, 7.2 Hz, 1H), 2.14 (d, J = 10.5 Hz, 1H), |
| | | | 2.08-1.98 (m, 1H), 1.97-1.84 (m, 4H), 1.66 |
| | | | (q, J = 13.0, 10.1 Hz, 4H), 0.95 (s, 9H). |
| 181 | (2S,4R)-1-((S)-2-(3-(4-(2-((4-(2- | 946.5 | ¹H NMR (300 MHz, DMSO-d⁶) δ 9.04 (s, 1H), |
| | ((3-amino-6-(2- | | 8.66 (t, J = 6.0 Hz, 1H), 8.57 (d, J = 9.3 Hz, |
| | hydroxyphenyl)pyridazin-4- | | 1H), 8.39 (t, J = 6.1 Hz, 1H), 8.28 (s, 1H), 8.02 |
| | yl)oxy)ethyl)benzyl)amino)-2- | | (d, J = 7.9 Hz, 1H), 7.67 (s, 1H), 7.45 (dd, J = |
| | oxoethyl)piperidin-1- | | 14.9, 6.1 Hz, 6H), 7.28 (t, J = 8.3 Hz, 3H), 6.95 |
| | yl)propanamido)-3,3- | | (d, J = 7.9 Hz, 2H), 6.58 (s, 2H), 4.61 (d, J = |
| | dimethylbutanoyl)-4-hydroxy-N- | | 9.3 Hz, 1H), 4.47 (dt, J = 17.8, 6.9 Hz, 5H), |
| | (4-(4-methylthiazol-5- | | 4.37-4.23 (m, 4H), 3.80-3.65 (m, 4H), 3.19 |
| | yl)benzyl)pyrrolidine-2- | | (t, J = 6.8 Hz, 2H), 2.99 (t, J = 12.9 Hz, 2H), |
| | carboxamide | | 2.63 (d, J = 11.9 Hz, 1H), 2.51 (s, 3H), 2.43- |
| | | | 2.32 (m, 1H), 2.18-1.90 (m, 6H), 1.70 (d, J = |
| | | | 13.6 Hz, 3H), 1.31 (t, J = 11.7 Hz, 2H), 1.02 (s, |
| | | | 9H). |
| 182 | (2S,4R)-1-[(2S)-2-[3-[3-([[4-(2- | 903.15 | ¹H NMR (300 MHz, DMSO-d⁶) δ14.38 (s, 1H), |
| | [[3-amino-6-(2- | | 8.98 (s, 1H), 8.57 (t, J = 6.0 Hz, 1H), 8.38- |
| | hydroxyphenyl)pyridazin-4- | | 8.23 (m, 2H), 7.95 (d, J = 7.9 Hz, 1H), 7.60 (s, |
| | yl]oxy]ethyl)phenyl]meth- | | 1H), 7.47-7.31 (m, 6H), 7.21 (t, J = 8.4 Hz, |
| | yl]carbamoyl)azetidin-1- | | 3H), 6.93-6.81 (m, 2H), 6.51 (s, 2H), 5.14 (s, |
| | yl]propanamido]-3,3- | | 1H), 4.56-4.32 (m, 6H), 4.30-4.17 (m, 3H), |
| | dimethylbutanoyl]-4-hydroxy-N- | | 3.70-3.62 (m, 2H), 3.40 (s, 2H), 3.22-3.07 |
| | [[4-(4-methyl-1,3-thiazol-5- | | (m, 6H), 2.59-2.51 (m, 1H), 2.45 (s, 3H), 2.29- |
| | yl)phenyl]methyl]pyrrolidine-2- | | 1.97 (m, 3H), 1.97-1.83 (m, 1H), 0.95 (s, |
| | carboxamide | | 9H). |
| 183 | (2S,4R)-1-[(2S)-2-(3-[3-[([[4-(2- | 918.45 | ¹H NMR (400 MHz, DMSO-d⁶) δ 14.38 (s, 1H), |
| | [[3-amino-6-(2- | | 8.97 (s, 1H), 8.57 (t, J = 6.1 Hz, 1H), 8.39 (d, |
| | hydroxyphenyl)pyridazin-4- | | J = 9.3 Hz, 1H), 8.30 (t, J = 5.9 Hz, 1H), 7.94 |
| | yl]oxy]ethyl)phenyl]meth- | | (dd, J = 8.1, 1.6 Hz, 1H), 7.59 (s, 1H), 7.47- |
| | yl]carbamoyl)methyl]azetidin-1- | | 7.30 (m, 6H), 7.26-7.14 (m, 3H), 6.91-6.82 |
| | yl]propanamido)-3,3- | | (m, 2H), 6.51 (s, 2H), 5.13 (s, 1H), 4.55-4.31 |
| | dimethylbutanoyl]-4-hydroxy-N- | | (m, 6H), 4.28-4.17 (m, 3H), 3.71-3.59 (m, |
| | [[4-(4-methyl-1,3-thiazol-5- | | 2H), 3.32-3.30 (m, 2H), 3.11 (t, J = 6.8 Hz, |
| | yl)phenyl]methyl]pyrrolidine-2- | | 2H), 2.85-2.81 (m, 2H), 2.69-2.52 (m, 3H), |
| | carboxamide | | 2.47-2.36 (m, 5H), 2.30-2.16 (m, 1H), 2.18- |
| | | | 2.07 (m, 1H), 2.08-1.97 (m, 1H), 1.95- |
| | | | 1.83 (m, 1H), 0.94 (s, 9H). |
| 184 | (1R,5S,6S)-N-[[4-(2-[[3-amino- | 930.20 | ¹H NMR (400 MHz, DMSO-d⁶) δ 14.39 (s, 1H), |
| | 6-(2-hydroxyphenyl)pyridazin- | | 8.98 (s, 1H), 8.55 (t, J = 5.9 Hz, 1H), 8.32 (t, |
| | 4-yl]oxy]ethyl)phenyl]methyl]-3- | | J = 6.0 Hz, 1H), 8.14 (d, J = 9.4 Hz, 1H), 7.94 (d, |
| | (2-[[(2S)-1-[(2S,4R)-4-hydroxy- | | J = 7.9 Hz, 1H), 7.59 (s, 1H), 7.35 (d, J = 19.2 |
| | 2-([[4-(4-methyl-1,3-thiazol-5- | | Hz, 6H), 7.23 (t, J = 7.7 Hz, 3H), 6.86 (t, J = |
| | yl)phenyl]methyl]carbamoyl)pyrrolidin- | | 8.8 Hz, 2H), 6.51 (s, 2H), 5.18 (d, J = 3.4 Hz, |
| | 1-yl]-3,3-dimethyl-1-oxobutan-2- | | 1H), 4.55 (d, J = 9.4 Hz, 1H), 4.42 (t, J = 7.2 |
| | yl]carbamoyl]ethyl)- | | Hz, 3H), 4.36 (d, J = 6.5 Hz, 2H), 4.29-4.15 |
| | 3-azabicyclo[3.1.0]hexane-6- | | (m, 3H), 3.70-3.59 (m, 2H), 3.11 (t, J = 6.7 Hz, |
| | carboxamide | | 2H), 2.97 (t, J = 9.0 Hz, 2H), 2.72-2.63 (m, |
| | | | 2H), 2.62-2.59 (m, 3H), 2.36 (d, J = 10.4 Hz, |
| | | | 2H), 2.30-2.21 (m, 2H), 2.03 (t, J = 10.4 Hz, |
| | | | 1H), 1.96 (s, 1H), 1.90 (d, J = 6.8 Hz, 2H), 1.72 |
| | | | (s, 1H), 0.93 (s, 9H). |
| 185 | N-[[4-(2-[[3-amino-6-(2- | 948.50 | ¹H NMR (400 MHz, DMSO-d⁶) δ 14.56 (s, 1H), |
| | hydroxyphenyl)pyridazin-4- | | 8.96 (s, 1H), 8.57 (t, J = 6.2 Hz, 1H), 8.47 (d, |
| | yl]oxy]ethyl)phenyl]methyl]-4- | | J = 9.3 Hz, 1H), 8.22 (dd, J = 11.9, 5.0 Hz, 2H), |
| | hydroxy-1-(2-[[(2S)-1-[(2S,4R)- | | 7.95 (dd, J = 8.1, 1.6 Hz, 1H), 7.59 (s, 1H), |
| | 4-hydroxy-2-([[4-(4-methyl-1,3- | | 7.40 (s, 4H), 7.32 (d, J = 8.0 Hz, 2H), 7.26- |
| | thiazol-5-yl)phenyl]meth- | | 7.13 (m, 3H), 6.86 (t, J = 8.1 Hz, 2H), 6.51 (s, |
| | yl]carbamoyl)pyrrolidin-1- | | 2H), 5.19 (d, J = 48.3 Hz, 2H), 4.52 (d, J = 9.4 |
| | yl]-3,3-dimethyl-1-oxobutan-2- | | Hz, 1H), 4.47-4.33 (m, 5H), 4.29-4.12 (m, |
| | yl]carbamoyl]ethyl)piperidine-4- | | 3H), 3.66 (dd, J = 10.1, 6.2 Hz, 2H), 3.51 (s, |
| | carboxamide | | 2H), 3.11 (t, J = 6.9 Hz, 2H), 2.70 (s, 2H), 2.43 |
| | | | (s, 5H), 2.25 (d, J = 16.9 Hz, 2H), 2.11-1.85 |
| | | | (m, 4H), 1.45 (d, J = 12.8 Hz, 2H), 0.95 (s, 9H). |
| 186 | N-[[4-(2-[[3-amino-6-(2- | 972.50 | ¹H NMR (400 MHz, DMSO-d6) δ 14.34 (s, 1H), |
| | hydroxyphenyl)pyridazin-4- | | 8.94 (s, 1H), 8.68 (d, J = 9.5 Hz, 1H), 8.62 (t, |
| | yl]oxy]ethyl)phenyl]methyl]-7- | | J = 6.1 Hz, 1H), 8.24 (s, 1H), 8.15 (t, J = 6.0 Hz, |
| | (2-[[(2S)-1-[(2S,4R)-4-hydroxy- | | 1H), 7.94 (dd, J = 8.0, 1.6 Hz, 1H), 7.59 (s, |
| | 2-([[4-(4-methyl-1,3-thiazol-5- | | 1H), 7.43 (d, J = 8.1 Hz, 2H), 7.40-7.31 (m, |
| | yl)phenyl]methyl]carbamoyl)pyrrolidin- | | 4H), 7.27-7.23 (m, 1H), 7.22-7.13 (m, 2H), |
| | 1-yl]-3,3-dimethyl-1-oxobutan-2- | | 6.87 (t, J = 8.1 Hz, 2H), 6.51 (s, 2H), 5.68- |
| | yl]carbamoyl]ethyl)-7- | | 4.83 (m, 1H), 4.54 (d, J = 9.5 Hz, 1H), 4.48- |
| | azaspiro[3.5]nonane-2- | | 4.39 (m, 4H), 4.38-4.32 (m, 1H), 4.25-4.16 |
| | carboxamide | | (m, 3H), 3.66-3.60 (m, 3H), 3.11 (t, J = 6.9 |
| | | | Hz, 2H), 2.96 (q, J = 8.6 Hz, 1H), 2.47-2.14 |
| | | | (m, 10H), 2.08-1.99 (m, 1H), 1.95-1.79 (m, |
| | | | 5H), 1.68-1.45 (m, 4H), 0.94 (s, 9H). |
| 188 | (2S,4R)-1-[(2S)-2-(3-[6-[2-([[4- | 973.20 | ¹H NMR (400 MHz, DMSO-d⁶) δ 8.93 (s, 1H), |
| | (2-[[3-amino-6-(2- | | 8.59 (t, J = 6.1 Hz, 1H), 8.29 (s, 1H), 7.88 (dd, |
| | hydroxyphenyl)pyridazin-4- | | J = 8.3, 1.6 Hz, 1H), 7.52 (s, 1H), 7.44-7.29 |
| | yl]oxy]ethyl)phenyl]meth- | | (m, 6H), 7.28-7.16 (m, 3H), 6.88 (td, J = 6.2, |
| | yl]carbamoyl)ethyl]-2,6- | | 5.7, 2.7 Hz, 2H), 5.68 (s, 1H), 4.49-4.32 (m, |
| | diazaspiro[3.3]heptan-2- | | 6H), 4.22 (d, J = 9.3 Hz, 3H), 3.36 (d, J = 9.0 |
| | yl]propanamido)-3,3- | | Hz, 8H), 3.11 (t, J = 6.4 Hz, 2H), 2.71-2.60 |
| | dimethylbutanoyl]-4-hydroxy-N- | | m, 4H), 2.42 (s, 3H), 2.29-2.12 (m, 4H), 2.04 |
| | [[4-(4-methyl-1,3-thiazol-5- | | (s, 2H), 1.95-1.84 (m, 1H), 0.91 (s, 9H). |
| | yl)phenyl]methyl]pyrrolidine-2- | | |
| | carboxamide | | |
| 189 | (2S,4R)-1-[(2S)-2-(3-[4-[2-([[4- | 961.50 | ¹H NMR (400 MHz, DMSO-d₆) δ 14.39 (s, 1H), |
| | (2-[[3-amino-6-(2- | | 8.97 (s, 1H), 8.59 (t, J = 6.2 Hz, 1H), 8.48- |
| | hydroxyphenyl)pyridazin-4- | | 8.36 (m, 2H), 8.14 (s, 1H), 7.97-7.90 (m, 1H), |
| | yl]oxy]ethyl)phenyl]meth- | | 7.59 (s, 1H), 7.48-7.32 (m, 7H), 7.22 (d, J = |
| | yl]carbamoyl)ethyl]piperazin-1- | | 7.8 Hz, 3H), 6.87 (t, J = 7.9 Hz, 2H), 6.50 (s, |
| | yl]propanamido)-3,3- | | 2H), 5.14 (d, J = 3.5 Hz, 1H), 4.54 (d, J = 9.3 |
| | dimethylbutanoyl]-4-hydroxy-N- | | Hz, 1H), 4.43 (p, J = 6.1, 5.2 Hz, 4H), 4.35 (s, |
| | [[4-(4-methyl-1,3-thiazol-5- | | 1H), 4.22 (dd, J = 19.9, 5.7 Hz, 3H), 3.69- |
| | yl)phenyl]methyl]pyrrolidine-2- | | 3.58 (m, 2H), 3.13-3.07 (m, 2H), 2.63-2.53 |
| | carboxamide | | (m, 5H), 2.44 (s, 4H), 2.41 (s, 2H), 2.28 (s, 3H), |
| | | | 2.02 (d, J = 8.9 Hz, 1H), 1.94-1.86 (m, |
| | | | 1H), 1.24 (s, 1H), 0.94 (s, 9H). 0.93 (s, 1H). |
| 187 | (2S,4R)-1-((S)-2-(3-((4-(2-((3- | 821.35 | ¹H NMR (400 MHz, DMSO-d6) δ 14.422- |
| | amino-6-(2- | | 14.352 (m, 1H), 8.98 (s, 1H), 8.58 (t, J = 6.1 |
| | hydroxyphenyl)pyridazin-4- | | Hz, 1H), 8.36 (d, J = 9.3 Hz, 1H), 8.20 (s, 1H), |
| | yl)oxy)ethyl)benzyl)amino)propanamido)- | | 7.94 (d, J = 7.9 Hz, 1H), 7.59 (s, 1H), 7.38 (dt, |
| | 3,3-dimethylbutanoyl)-4-hydroxy-N- | | J = 23.4, 7.9 Hz, 8H), 7.23 (t, J = 7.7 Hz, 1H), |
| | (4-(4-methylthiazol-5- | | 6.87 (t, J = 8.3 Hz, 2H), 6.51 (s, 2H), 5.13 (s, |
| | yl)benzyl)pyrrolidine-2- | | 1H), 4.54 (d, J = 9.2 Hz, 1H), 4.43 (t, J = 7.5 |
| | carboxamide | | Hz, 4H), 4.35 (s, 1H), 4.22 (dd, J = 15.9, 5.5 |
| | | | Hz, 1H), 3.76 (s, 2H), 3.72-3.56 (m, 3H), 3.11 |
| | | | (t, J = 6.9 Hz, 2H), 2.75 (d, J = 8.2 Hz, 2H), |
| | | | 2.46-2.31 (m, 5H), 2.03 (t, J = 9.9 Hz, 1H), |
| | | | 1.91 (dt, J = 13.3, 6.9 Hz, 1H), 0.93 (s, 9H). |
| 199 | (2S,4R)-1-((S)-2-(3-(3-(1-(4-(2- | 958.46 | ¹H NMR (400 MHz, DMSO-d6) δ 14.37 (s, 1H), |
| | ((3-amino-6-(2- | | 8.96 (s, 1H), 8.57 (t, J = 6.1 Hz, 1H), 8.44 (d, |
| | hydroxyphenyl)pyridazin-4- | | J = 9.3 Hz, 1H), 7.93 (dd, J = 8.1, 1.6 Hz, 1H), |
| | yl)oxy)ethyl)benzoyl)piperidin- | | 7.60 (s, 1H), 7.48-7.35 (m, 6H), 7.33-7.27 |
| | 4-yl)azetidin-1- | | (m, 2H), 7.23 (ddd, J = 8.5, 7.2, 1.6 Hz, 1H), |
| | yl)propanamido)-3,3- | | 6.88 (d, J = 7.9 Hz, 2H), 6.52 (s, 2H), 5.12 (d, |
| | dimethylbutanoyl)-4-hydroxy-N- | | J = 3.5 Hz, 1H), 4.57-4.31 (m, 7H), 4.23 (dd, |
| | (4-(4-methylthiazol-5- | | J = 15.8, 5.5 Hz, 1H), 3.69-3.59 (m, 2H), 3.45 |
| | yl)benzyl)pyrrolidine-2- | | (s, 2H), 3.31 (s, 3H), 3.18 (t, J = 6.8 Hz, 2H), |
| | carboxamide | | 3.08-2.81, (m, 3H), 2.81-2.59 (m, 3H), 2.43 |
| | | | (s, 3H), 2.18 (d, J = 31.9 Hz, 3H), 2.01 (d, J = |
| | | | 8.8 Hz, 1H), 1.90 (ddd, J = 12.8, 8.8, 4.6 Hz, |
| | | | 1H), 1.65 (s, 3H), 0.94 (s, 9H). |
| 216 | (2S,4R)-1-((2S)-2-(3-(3-(4-(2- | 930.45 | ¹H NMR (400 MHz, DMSO-d6) δ 14.39 (s, 1H), |
| | ((3-amino-6-(2- | | 8.96 (s, 1H), 8.84-8.50 (m, 2H), 7.94 (d, J = |
| | hydroxyphenyl)pyridazin-4- | | 7.9 Hz, 1H), 7.60 (s, 1H), 7.45-7.33 (m, 6H), |
| | yl)oxy)ethyl)benzoyl)-3,8- | | 7.27-7.17 (m, 3H), 6.87 (t, J = 7.8 Hz, 2H), |
| | diazabicyclo[3.2.1]octan-8- | | 6.54 (s, 2H), 5.15 (s, 1H), 4.59 (d, J = 9.4 Hz, |
| | yl)propanamido)-3,3- | | 1H), 4.53-4.27 (m, 5H), 4.23 (dd, J = 16.1, |
| | dimethylbutanoyl)-4-hydroxy-N- | | 5.5 Hz, 2H), 3.66 (dd, J = 15.7, 11.9 Hz, 2H), |
| | (4-(4-methylthiazol-5- | | 3.15 (s, 5H), 2.97 (s, 1H), 2.42 (s, 3H), 2.24 (s, |
| | yl)benzyl)pyrrolidine-2- | | 2H), 2.03 (s, 1H), 1.98-1.71 (m, 4H), 1.53 (s, |
| | carboxamide | | 2H), 1.28 (d, J = 37.8 Hz, 2H), 0.96 (s, 9H). |
| 200 | (2S,4R)-1-((S)-2-(3-(6-(4-(2-((3- | 916.40 | ¹H NMR (400 MHz, DMSO-d6) δ 14.38 (s, 1H), |
| | amino-6-(2- | | 8.97 (s, 1H), 8.57 (t, J = 6.0 Hz, 1H), 8.25 (d, |
| | hydroxyphenyl)pyridazin-4- | | J = 9.3 Hz, 1H), 7.94 (dd, J = 8.2, 1.5 Hz, 1H), |
| | yl)oxy)ethyl)benzoyl)-2,6- | | 7.62-7.53 (m, 3H), 7.47 (d, J = 8.3 Hz, 2H), |
| | diazaspiro[3.3]heptan-2- | | 7.44-7.35 (m, 4H), 7.23 (td, J = 7.7, 7.1, 1.6 |
| | yl)propanamido)-3,3- | | Hz, 1H), 6.87 (t, J = 7.6 Hz, 2H), 6.53 (s, 2H), |
| | dimethylbutanoyl)-4-hydroxy-N- | | 5.13 (d, J = 3.5 Hz, 1H), 4.56-4.17 (m, 10H), |
| | (4-(4-methylthiazol-5- | | 4.08 (s, 2H), 3.71-3.59 (m, 2H), 3.30-3.11 |
| | yl)benzyl)pyrrolidine-2- | | (m, 7H), 2.44 (s, 3H), 2.27-2.17 (m, 1H), 2.16- |
| | carboxamide | | 2.06 (m, 1H), 2.07-1.98 (m, 1H), 1.95- |
| | | | 1.84 (m, 1H), 0.94 (s, 9H). |
| 201 | (2S,4R)-1-((S)-2-(3-(9-(4-(2-((3- | 972.50 | 1H NMR (400 MHz, DMSO-d6) δ 14.39 (s, 1H), |
| | amino-6-(2- | | 8.92 (s, 1H), 8.67 (d, J = 46.2 Hz, 2H), 7.93 (d, |
| | hydroxyphenyl)pyridazin-4- | | J = 7.9 Hz, 1H), 7.59 (s, 1H), 7.45 (dd, J = |
| | yl)oxy)ethyl)benzoyl)-3,9- | | 16.2, 7.9 Hz, 4H), 7.32 (dd, J = 19.7, 7.8 Hz, |
| | diazaspiro[5.5]undecan-3- | | 4H), 7.26-7.18 (m, 1H), 6.87 (t, J = 7.9 Hz, |
| | yl)propanamido)-3,3- | | 2H), 6.54 (s, 2H), 5.14 (d, J = 3.4 Hz, 1H), 4.48 |
| | dimethylbutanoyl)-4-hydroxy-N- | | (m, 5H), 4.36 (s, 1H), 4.18 (dd, J = 16.0, 5.4 |
| | (4-(4-methylthiazol-5- | | Hz, 1H), 3.64 (q, J = 10.7, 9.6 Hz, 3H), 3.51 |
| | yl)benzyl)pyrrolidine-2- | | (m, 2H), 3.24-3.12 (m, 5H), 2.67 (s, 2H), 2.41 |
| | carboxamide | | (s, 7H), 2.03 (t, J = 10.5 Hz, 1H), 1.89 (d, J = |
| | | | 15.2 Hz, 1H), 1.63-1.27 (m, 8H), 0.95 (s, 9H). |
| 202 | (2S,4R)-1-((S)-2-(3-(4-((1-(4-(2- | 1002.45 | ¹H NMR (300 MHz, DMSO-d6) δ 14.39 (s, 1H), |
| | ((3-amino-6-(2- | | 8.94 (s, 1H), 8.78 (d, J = 9.5 Hz, 1H), 8.63 (t, |
| | hydroxyphenyl)pyridazin-4- | | J = 6.1 Hz, 1H), 7.99-7.90 (m, 1H), 7.61 (s, |
| | yl)oxy)ethyl)benzoyl)piperidin- | | 1H), 7.50-7.42 (m, 4H), 7.40-7.30 (m, 4H), |
| | 4-yl)oxy)piperidin-1- | | 7.24 (td, J = 7.6, 1.5 Hz, 1H), 6.91-6.83 (m, |
| | yl)propanamido)-3,3- | | 2H), 6.54 (s, 2H), 5.15 (s, 1H), 4.59-4.40 (m, |
| | dimethylbutanoyl)-4-hydroxy-N- | | 5H), 4.39-4.33 (m, 1H), 4.24-4.14 (m, 1H), |
| | (4-(4-methylthiazol-5- | | 3.99-3.83 (m, 1H), 3.70-3.57 (m, 3H), 3.48- |
| | yl)benzyl)pyrrolidine-2- | | 3.41 (m, 2H), 3.24-3.16 (m, 3H), 3.15- |
| | carboxamide | | 3.01 (m, 2H), 2.80-2.65 (m, 2H), 2.47-2.40 |
| | | | (m, 5H), 2.39-2.32 (m, 1H), 2.28-2.14 (m, |
| | | | 2H), 2.12-2.01 (m, 2H), 1.92-1.77 (m, 3H), |
| | | | 1.71-1.63 (m, 1H), 1.59-1.46 (m, 2H), 1.34- |
| | | | 1.22 (m, 2H), 0.95 (s, 9H). |
| 203 | (2S,4R)-1-((2S)-2-(3-(5-(4-(2- | 930.20 | ¹H NMR (400 MHz, DMSO-d6) δ 14.38 (s, 1H), |
| | ((3-amino-6-(2- | | 8.97 (s, 1H), 8.51 (s, 1H), 8.32 (d, J = 9.4 Hz, |
| | hydroxyphenyl)pyridazin-4- | | 1H), 7.93 (d, J = 7.9 Hz, 1H), 7.60 (s, 1H), 7.51- |
| | yl)oxy)ethyl)benzoyl)hexahydro- | | 7.29 (m, 8H), 7.28-7.16 (m, 1H), 6.86 (t, J = |
| | pyrrolo[3,4-c]pyrrol-2(1H)- | | 8.1 Hz, 2H), 6.54 (s, 2H), 5.14 (s, 1H), 4.56 (d, |
| | yl)propanamido)-3,3- | | J = 9.5 Hz, 1H), 4.52-4.38 (m, 3H), 4.31 (d, |
| | dimethylbutanoyl)-4-hydroxy-N- | | J = 28.8 Hz, 3H), 3.84 (s, 1H), 3.71-3.48 (m, |
| | (4-(4-methylthiazol-5- | | 4H), 3.17 (t, J = 6.7 Hz, 4H), 2.74 (s, 2H), 2.44 |
| | yl)benzyl)pyrrolidine-2- | | (d, J = 12.0 Hz, 9H), 2.30-2.19 (m, 1H), 1.96 |
| | carboxamide | | (dd, J = 47.9, 6.4 Hz, 2H), 0.94 (s, 9H). |
| 226 | (2S,4R)-1-((2S)-2-(3-(8-(4-(2- | 930.40 | ¹H NMR (400 MHz, DMSO-d6) δ 14.37 (s, 1H), |
| | ((3-amino-6-(2- | | 8.98 (s, 1H), 8.57 (s, 1H), 8.02 (d, J = 9.5 Hz, |
| | hydroxyphenyl)pyridazin-4- | | 1H), 7.96-7.91 (m, 1H), 7.60 (s, 1H), 7.48 (d, |
| | yl)oxy)ethyl)benzoyl)-3,8- | | J = 8.0 Hz, 2H), 7.44-7.36 (m, 6H), 7.23 (td, |
| | diazabicyclo[3.2.1]octan-3- | | J = 7.6, 1.6 Hz, 1H), 6.87 (dd, J = 8.0, 6.9 Hz, |
| | yl)propanamido)-3,3- | | 2H), 6.53 (s, 2H), 5.12 (d, J = 3.3 Hz, 1H), 4.60- |
| | dimethylbutanoyl)-4-hydroxy-N- | | 4.34 (m, 7H), 4.20 (dd, J = 15.8, 5.4 Hz, 1H), |
| | (4-(4-methylthiazol-5- | | 3.89 (s, 1H), 3.68 (dd, J = 10.5, 4.2 Hz, 1H), |
| | yl)benzyl)pyrrolidine-2- | | 3.63-3.57 (m, 1H), 3.19 (t, J = 6.8 Hz, 2H), |
| | carboxamide | | 2.86-2.75 (m, 1H), 2.63-2.54 (m, 2H), 2.46- |
| | | | 2.39 (m, 4H), 2.30-2.17 (m, 3H), 2.05- |
| | | | 1.98 (m, 1H), 1.90-1.69 (m, 5H), 1.34-1.27 |
| | | | (m, 1H), 0.95 (s, 9H). |
| 204 | (2S,4R)-1-((S)-2-(3-(4-(1-(4-(2- | 958.15 | ¹H NMR (400 MHz, DMSO-d6) δ 14.38 (s, 1H), |
| | ((3-amino-6-(2- | | 8.97 (s, 1H), 8.51 (s, 1H), 8.32 (d, J = 9.4 Hz, |
| | hydroxyphenyl)pyridazin-4- | | 1H), 7.93 (d, J = 7.9 Hz, 1H), 7.60 (s, 1H), 7.51- |
| | yl)oxy)ethyl)benzoyl)azetidin-3- | | 7.29 (m, 8H), 7.28-7.16 (m, 1H), 6.86 (t, J = |
| | yl)piperidin-1-yl)propanamido)- | | 8.1 Hz, 2H), 6.54 (s, 2H), 5.14 (s, 1H), 4.56 (d, |
| | 3,3-dimethylbutanoyl)-4- | | J = 9.5 Hz, 1H), 4.52-4.38 (m, 3H), 4.31 (d, |
| | hydroxy-N-(4-(4-methylthiazol- | | J = 28.8 Hz, 3H), 3.84 (s, 1H), 3.71-3.48 (m, |
| | 5-yl)benzyl)pyrrolidine-2- | | 4H), 3.17 (t, J = 6.7 Hz, 4H), 2.74 (s, 2H), 2.44 |
| | carboxamide | | (d, J = 12.0 Hz, 9H), 2.30-2.19 (m, 1H), 1.96 |
| | | | (dd, J = 47.9, 6.4 Hz, 2H), 0.94 (s, 9H). |
| 231 | (2S,4R)-1-((2S)-2-(3-(3-(4-(2- | 916.41 | ¹H NMR (300 MHz, DMSO-d6) δ 14.38 (s, 1H), |
| | ((3-amino-6-(2- | | 8.98 (s, 1H), 8.57 (t, J = 5.7 Hz, 1H), 8.04 (s, |
| | hydroxyphenyl)pyridazin-4- | | 1H), 7.95 (d, J = 7.9 Hz, 1H), 7.62 (s, 1H), 7.47 |
| | yl)oxy)ethyl)benzoyl)-3,6- | | (s, 4H), 7.40 (d, J = 4.3 Hz, 4H), 7.29-7.18 |
| | diazabicyclo[3.2.0]heptan-6- | | (m, 1H), 6.87 (m, J = 8.1, 6.9 Hz, 2H), 6.54 (s, |
| | yl)propanamido)-3,3- | | 2H), 5.12 (d, J = 3.4 Hz, 1H), 4.57-4.31 (m, |
| | dimethylbutanoyl)-4-hydroxy-N- | | 6H), 4.23 (d, J = 16.3 Hz, 1H), 3.98 (s, 1H), |
| | (4-(4-methylthiazol-5- | | 3.65 (s, 2H), 3.50 (s, 2H), 3.19 (t, J = 6.9 Hz, |
| | yl)benzyl)pyrrolidine-2- | | 3H), 3.00 (d, J = 41.0 Hz, 3H), 2.77-2.70 (m, |
| | carboxamide | | 1H), 2.44 (d, J = 3.0 Hz, 4H), 2.21 (s, 1H), 2.09- |
| | | | 1.82 (m, 3H), 0.93 (d, J = 9.8 Hz, 9H). |
| 241 | (2S,4R)-1-((2S)-2-(3-(6-(4-(2- | 915.90 | ¹H NMR (400 MHz, DMSO-d₆) δ 14.35 (s, 1H), |
| | ((3-amino-6-(2- | | 9.00-8.93 (m, 1H), 8.54 (s, 1H), 8.14 (s, 2H), |
| | hydroxyphenyl)pyridazin-4- | | 7.93 (s, 1H), 7.65-7.52 (m, 3H), 7.49-7.42 |
| | yl)oxy)ethyl)benzoyl)-3,6- | | (m, 1H), 7.41-7.31 (m, 4H), 7.23 (t, J = 7.8 |
| | diazabicyclo[3.2.0]heptan-3- | | Hz, 1H), 6.87 (t, J = 7.6 Hz, 2H), 6.50 (s, 2H), |
| | yl)propanamido)-3,3- | | 5.12-5.09 (m, 1H), 5.03-4.75 (m, 1H), 4.61- |
| | dimethylbutanoyl)-4-hydroxy-N- | | 4.31 (m, 6H), 4.26-4.12 (m, 2H), 4.01- |
| | (4-(4-methylthiazol-5- | | 3.90 (m, 1H), 3.65 (s, 2H), 3.17 (d, J = 6.9 Hz, |
| | yl)benzyl)pyrrolidine-2- | | 3H), 3.04 (s, 2H), 2.89 (s, 1H), 2.38 (t, J = 8.7 |
| | carboxamide | | Hz, 3H), 2.12-1.88 (m, 5H), 1.25 (s, 1H), 1.05- |
| | | | 0.68 (m, 9H). |
| 219 | (2S,4R)-1-((S)-2-(3-(5-(4-(2-((3- | 928.35 | ¹H NMR (400 MHz, DMSO-d⁶) δ 14.37 (s, 1H), |
| | amino-6-(2- | | 8.97 (s, 1H), 8.57 (t, J = 6.1 Hz, 1H), 8.28 (d, |
| | hydroxyphenyl)pyridazin-4- | | J = 9.4 Hz, 1H), 7.93 (d, J = 8.2 Hz, 1H), 7.61 (s, |
| | yl)oxy)ethyl)benzoyl)-3,4,5,6- | | 1H), 7.54-7.44 (m, 4H), 7.44-7.35 (m, 4H), |
| | tetrahydropyrrolo[3,4-c]pyrrol- | | 7.23 (td, J = 7.6, 1.5 Hz, 1H), 6.92-6.84 (m, |
| | 2(1H)-yl)propanamido)-3,3- | | 2H), 6.53 (s, 2H), 5.12 (d, J = 3.5 Hz, 1H), 4.57- |
| | dimethylbutanoyl)-4-hydroxy-N- | | 4.46 (m, 3H), 4.45-4.31 (m, 3H), 4.30- |
| | (4-(4-methylthiazol-5- | | 4.17 (m, 3H), 4.10 (s, 2H), 3.71-3.59 (m, 2H), |
| | yl)benzyl)pyrrolidine-2- | | 3.55-3.45 (m, 2H), 3.43-3.37 (m, 2H), 3.19 |
| | carboxamide | | (t, J = 6.7 Hz, 2H), 2.92-2.79 (m, 2H), 2.44 (s, |
| | | | 3H), 2.42-2.25 (m, 2H), 2.07-1.97 (m, 1H), |
| | | | 1.95-1.84 (m, 1H), 0.93 (s, 9H). |
| 224 | (2S,4R)-1-((S)-2-(3-(1′-(4-(2- | 930.43 | |
| | ((3-amino-6-(2- | | |
| | hydroxyphenyl)pyridazin-4- | | |
| | yl)oxy)ethyl)benzoyl)-[3,3′- | | |
| | biazetidin]-1-yl)propanamido)- | | |
| | 3,3-dimethylbutanoyl)-4- | | |
| | hydroxy-N-(4-(4-methylthiazol- | | |
| | 5-yl)benzyl)pyrrolidine-2- | | |
| | carboxamide | | |
| 232 | (2S,4R)-1-((2S)-2-(3-(4-(2-((4- | 985.55 | ¹H NMR (400 MHz, DMSO-d⁶) δ 14.14 (s, 1H), |
| | (3-(3-amino-6-(2- | | 8.95 (s, 1H), 8.60 (t, J = 6.1 Hz, 1H), 8.52 (d, |
| | hydroxyphenyl)pyridazin-4-yl)- | | J = 9.4 Hz, 1H), 7.90 (dd, J = 8.2, 1.6 Hz, 1H), |
| | 3,8-diazabicyclo[3.2.1]octan-8- | | 7.77 (d, J = 6.0 Hz, 1H), 7.51-7.32 (m, 5H), |
| | yl)pyridin-2- | | 7.26-7.17 (m, 1H), 6.90-6.79 (m, 2H), 6.53 |
| | yl)oxy)ethyl)piperazin-1- | | (dd, J = 6.1, 2.1 Hz, 1H), 6.12 (d, J = 2.0 Hz, |
| | yl)propanamido)-3,3- | | 1H), 5.97 (s, 2H), 5.14 (d, J = 3.5 Hz, 1H), 4.58- |
| | dimethylbutanoyl)-4-hydroxy-N- | | 4.38 (m, 5H), 4.38-4.32 (m, 1H), 4.31- |
| | (4-(4-methylthiazol-5- | | 4.13 (m, 3H), 3.72-3.54 (m, 2H), 3.30-3.21 |
| | yl)benzyl)pyrrolidine-2- | | (m, 4H), 3.00 (d, J = 11.6 Hz, 2H), 2.62-2.57 |
| | carboxamide | | (m, 2H), 2.56-2.51 (m, 4H), 2.46-2.41 (m, |
| | | | 5H), 2.40-2.31 (m, 3H), 2.31-2.21 (m, 1H), |
| | | | 2.21-2.12 (m, 2H), 2.08-1.85 (m, 4H), 0.94 |
| | | | (s, 9H). |
| 252 | (2S,4R)-1-((S)-2-(3-(9-(3-((4-(2- | 1029 | ¹H NMR (300 MHz, DMSO-d6) δ 8.97 (s, 1H), |
| | ((3-amino-6-(2- | | 8.80 (d, J = 9.4 Hz, 1H), 8.63 (t, J = 6.1 Hz, |
| | hydroxyphenyl)pyridazin-4- | | 1H), 8.42 (t, J = 5.8 Hz, 1H), 7.99-7.90 (m, |
| | yl)oxy)ethyl)benzyl)amino)-3- | | 1H), 7.60 (s, 1H), 7.45 (d, J = 8.1 Hz, 2H), 7.39- |
| | oxopropyl)-3,9- | | 7.30 (m, 4H), 7.24 (dq, J = 7.0, 2.8, 2.2 Hz, |
| | diazaspiro[5.5]undecan-3- | | 3H), 6.87 (t, J = 7.6 Hz, 2H), 6.50 (s, 2H), 5.11 |
| | yl)propanamido)-3,3- | | (s, 1H), 4.59-4.32 (m, 6H), 4.29-4.11 (m, |
| | dimethylbutanoyl)-4-hydroxy-N- | | 3H), 3.75-3.56 (m, 3H), 3.12 (t, J = 6.8 Hz, |
| | (4-(4-methylthiazol-5- | | 2H), 2.43 (s, 4H), 2.36 (s, 5H), 2.28 (d, J = 6.3 |
| | yl)benzyl)pyrrolidine-2- | | Hz, 8H), 2.04 (t, J = 10.5 Hz, 1H), 1.91 (ddd, |
| | carboxamide | | J = 12.9, 8.6, 4.4 Hz, 1H), 1.44 (s, 4H), 1.39- |
| | | | 1.23 (m, 5H), 0.95 (s, 9H). |
| 238 | (2S,4R)-1-((S)-2-(3-(3-((1-(4-(2- | 974.25 | ¹H NMR (300 MHz, DMSO-d6) δ 14.39 (br s, |
| | ((3-amino-6-(2- | | 1H), 8.98 (s, 1H), 8.57 (t, J = 6.0 Hz, 1H), 8.28 (d, |
| | hydroxyphenyl)pyridazin-4- | | J = 9.3 Hz, 1H), 7.98-7.91 (m, 1H), 7.61 (s, |
| | yl)oxy)ethyl)benzoyl)piperidin- | | 1H), 7.51-7.30 (m, 8H), 7.24 (td, J = 7.6, 1.5 |
| | 4-yl)oxy)azetidin-1- | | Hz, 1H), 6.95-6.81 (m, 2H), 6.54 (s, 2H), 5.13 |
| | yl)propanamido)-3,3- | | (s, 1H), 4.58-4.05 (m, 8H), 3.95 (s, 1H), 3.73- |
| | dimethylbutanoyl)-4-hydroxy-N- | | 3.60 (m, 2H), 3.59-3.47 (m, 3H), 3.22-3.02 |
| | (4-(4-methylthiazol-5- | | (m, 5H), 2.84-2.69 (m, 1H), 2.58 (d, J = 6.3 Hz, |
| | yl)benzyl)pyrrolidine-2- | | 1H), 2.44 (s, 4H), 2.32-1.83 (m, 5H), 1.75 (s, |
| | carboxamide | | 2H), 1.37 (s, 2H), 0.94 (s, 9H). |
| 245 | (2S,4R)-1-((S)-2-(3-(4-((1-(4-(2- | 974.20 | ¹H NMR (300 MHz, DMSO-d6) δ 14.38 (s, 1H), |
| | ((3-amino-6-(2- | | 8.95 (s, 1H), 8.75-8.56 (m, 2H), 8.00-7.89 (m, |
| | hydroxyphenyl)pyridazin-4- | | 1H), 7.61 (s, 1H), 7.54 (s, 2H), 7.51-7.44 (m, |
| | yl)oxy)ethyl)benzoyl)azetidin-3- | | 2H), 7.44-7.36 (m, 4H), 7.29-7.19 (m, 1H), |
| | yl)oxy)piperidin-1- | | 6.94-6.83 (m, 2H), 6.53 (s, 2H), 5.14 (d, J = 3.4 |
| | yl)propanamido)-3,3- | | Hz, 1H), 4.60-4.30 (m, 8H), 4.29-4.12 (m, |
| | dimethylbutanoyl)-4-hydroxy-N- | | 2H), 4.08-3.97 (m, 1H), 3.83-3.54 (m, 3H), |
| | (4-(4-methylthiazol-5- | | 3.32-3.29 (m, 4H), 3.21 (t, J = 6.7 Hz, 2H), 2.90- |
| | yl)benzyl)pyrrolidine-2- | | 2.68 (m, 2H), 2.44 (s, 4H), 2.12-1.98 (m, 3H), |
| | carboxamide | | 1.97-1.74 (m, 3H), 1.68-1.37 (m, 2H), 0.95 (s, |
| | | | 9H). |
| 256 | (2S,4R)-1-((S)-2-(3-((11-((4-(2- | 1018.50 | ¹H NMR (300 MHz, DMSO-d6) δ 14.59 (s, 1H), |
| | ((3-amino-6-(2- | | 8.99 (s, 1H), 8.46-8.35 (m, 2H), 8.26 (t, J = |
| | hydroxyphenyl)pyridazin-4- | | 5.8 Hz, 1H), 7.95 (d, J = 8.1 Hz, 1H), 7.60 (s, |
| | yl)oxy)ethyl)benzyl)amino)-11- | | 1H), 7.48-7.30 (m, 6H), 7.28-7.14 (m, 3H), |
| | oxoundecyl)amino)propanamido)- | | 6.87 (dd, J = 8.1, 6.8 Hz, 2H), 6.51 (s, 2H), |
| | 3,3-dimethylbutanoyl)-4- | | 5.00-4.84 (m, 1H), 4.54-4.49 (d, 1H), 4.49- |
| | hydroxy-N-((S)-1-(4-(4- | | 4.38 (m, 3H), 4.28 (s, 1H), 4.23 (d, J = 5.9 Hz, |
| | methylthiazol-5- | | 2H), 3.59 (d, J = 7.2 Hz, 2H), 3.12 (t, J = 6.8 |
| | yl)phenyl)ethyl)pyrrolidine-2- | | Hz, 2H), 2.73 (s, 1H), 2.46 (s, 4H), 2.28 (p, J = |
| | carboxamide | | 1.8 Hz, 2H), 2.11 (t, J = 7.4 Hz, 2H), 2.00 (d, |
| | | | J = 9.2 Hz, 1H), 1.80 (dt, J = 8.8, 4.3 Hz, 1H), |
| | | | 1.58-1.44 (m, 4H), 1.43-1.54 (m, 4H), 1.24 |
| | | | (s, 14H), 0.94 (s, 9H). |
| 259 | (2S,4R)-1-((2S)-2-(3-(5-(3-((4- | 1001.50 | ¹H NMR (300 MHz, DMSO-d6) δ 14.39 (s, 1H), |
| | (2-((3-amino-6-(2- | | 8.99 (s, 1H), 8.54-8.23 (m, 3H), 8.01-7.86 (m, |
| | hydroxyphenyl)pyridazin-4- | | 1H), 7.60 (s, 1H), 7.48-7.30 (m, 6H), 7.30- |
| | yl)oxy)ethyl)benzyl)amino)-3- | | 7.16 (m, 3H), 6.98-6.66 (m, 2H), 6.51 (s, 2H), |
| | oxopropyl)hexahydropyrrolo[3,4- | | 5.11 (s, 1H), 5.01-4.83 (m, 1H), 4.51 (d, J = |
| | c]pyrrol-2(1H)- | | 9.4 Hz, 1H), 4.43 (d, J = 6.9 Hz, 3H), 4.25 (d, |
| | yl)propanamido)-3,3- | | J = 6.0 Hz, 3H), 3.60 (d, J = 4.2 Hz, 2H), 3.12 (t, |
| | dimethylbutanoyl)-4-hydroxy-N- | | J = 6.8 Hz, 2H), 2.57 (dd, J = 18.8, 6.0 Hz, 8H), |
| | ((S)-1-(4-(4-methylthiazol-5- | | 2.46 (s, 3H), 2.38 (d, J = 17.7 Hz, 2H), 2.33- |
| | yl)phenyl)ethyl)pyrrolidine-2- | | 2.11 (m, 7H), 2.05-1.92 (m, 1H), 1.88-1.72 (m, |
| | carboxamide | | 1H), 1.37 (d, J = 7.0 Hz, 3H), 0.94 (s, 9H). |
| 249 | (2S,4R)-1-((S)-2-(3-(4-((1-(3- | 1073.56 | ¹H NMR (400 MHz, DMSO-d6) δ 14.38 (s, 1H), |
| | ((4-(2-((3-amino-6-(2- | | 8.98 (s, 1H), 8.42 (t, J = 22.0 Hz, 3H), 7.94 (d, |
| | hydroxyphenyl)pyridazin-4- | | J = 8.0 Hz, 1H), 7.59 (s, 1H), 7.43 (d, J = 7.4 |
| | yl)oxy)ethyl)benzyl)amino)-3- | | Hz, 2H), 7.35 (d, J = 8.2 Hz, 4H), 7.23 (s, 3H), |
| | oxopropyl)piperidin-4- | | 6.88 (d, J = 8.4 Hz, 2H), 6.50 (s, 2H), 5.11 (s, |
| | yl)oxy)piperidin-1- | | 1H), 4.90 (s, 1H), 4.55-4.38 (m, 4H), 4.26 (t, |
| | yl)propanamido)-3,3- | | J = 7.6 Hz, 3H), 3.58 (s, 10H), 2.67-2.50 (m, |
| | dimethylbutanoyl)-4-hydroxy-N- | | 5H), 2.23-2.18 (m, 9H), 1.78 (s, 6H), 1.60- |
| | ((S)-1-(4-(4-methylthiazol-5- | | 1.29 (m, 7H), 1.24 (s, 1H), 0.94 (s, 9H). |
| | yl)phenyl)ethyl)pyrrolidine-2- | | |
| | carboxamide | | |
| 250 | (2S,4R)-1-((S)-2-(3-(9-(3-((4-(2- | 1043 | ¹H NMR (300 MHz, DMSO-d6) δ 14.4 (s, 1H), |
| | ((3-amino-6-(2- | | 8.99 (s, 1H), 8.61 (d, J = 9.3 Hz, 1H), 8.49- |
| | hydroxyphenyl)pyridazin-4- | | 8.34 (m, 2H), 7.99-7.90 (m, 1H), 7.60 (s, 1H), |
| | yl)oxy)ethyl)benzyl)amino)-3- | | 7.46-7.32 (m, 6H), 7.23 (dd, J = 8.5, 3.2 Hz, |
| | oxopropyl)-3,9- | | 3H), 6.87 (dd, J = 8.2, 6.9 Hz, 2H), 6.50 (s, |
| | diazaspiro[5.5]undecan-3- | | 2H), 5.11 (s, 1H), 4.91 (t, J = 7.1 Hz, 1H), 4.56- |
| | yl)propanamido)-3,3- | | 4.34 (m, 4H), 4.25 (d, J = 5.6 Hz, 3H), 3.60 |
| | dimethylbutanoyl)-4-hydroxy-N- | | (s, 2H), 3.13 (t, J = 6.8 Hz, 2H), 2.61 (s, 2H), |
| | ((S)-1-(4-(4-methylthiazol-5- | | 2.46 (s, 4H), 2.44-2.27 (m, 12H), 2.02 (t, J = |
| | yl)phenyl)ethyl)pyrrolidine-2- | | 10.8 Hz, 1H), 1.79 (s, 1H), 1.40 (t, J = 12.7 Hz, |
| | carboxamide | | 11H), 0.95 (s, 9H). |

### Preparation of (2S,4R)-1-((S)-2-(2-(9-(3-((4-(2-((3-amino-6-(2-hydroxyphenyl)pyridazin-4-yl)oxy)ethyl)benzyl)amino)-3-oxopropyl)-3,9-diazaspiro[5.5]undecan-3-yl)acetamido)-3,3-dimethylbutanoyl)-4-hydroxy-N-(4-(4-methylthiazol-5-yl)benzyl)pyrrolidine-2-carboxamide (Compound 266)

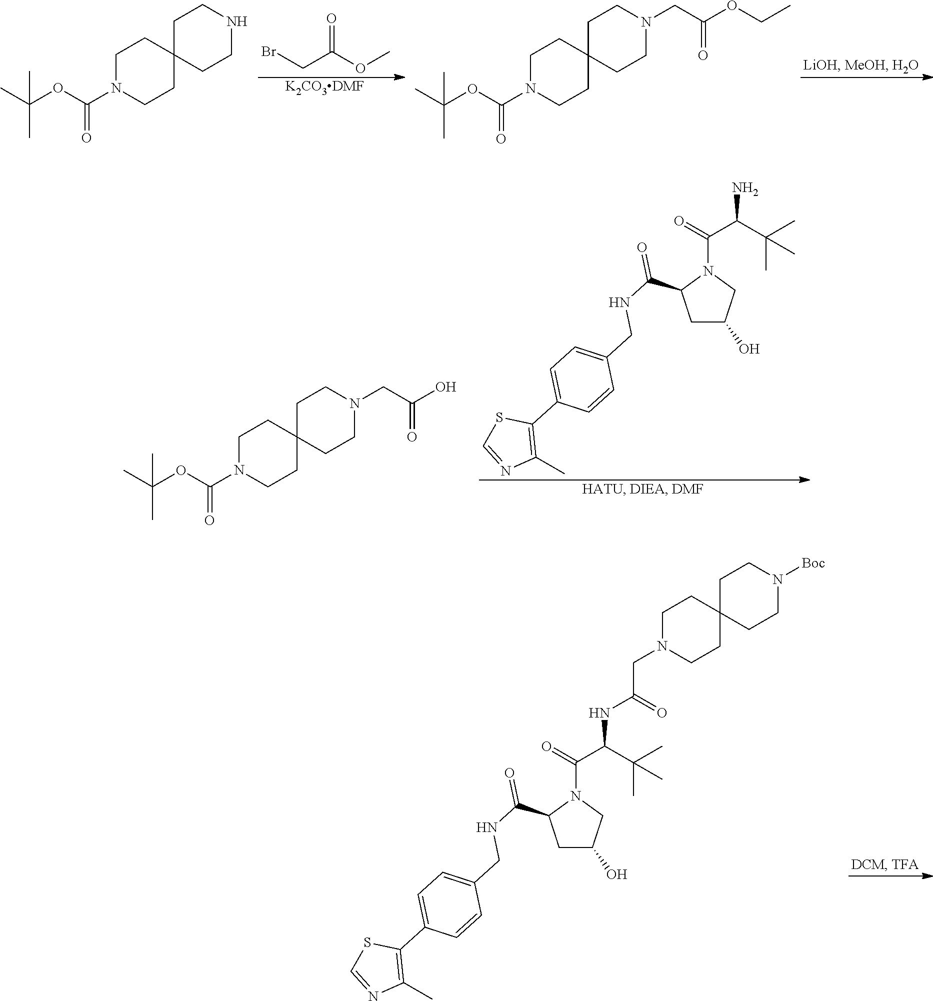

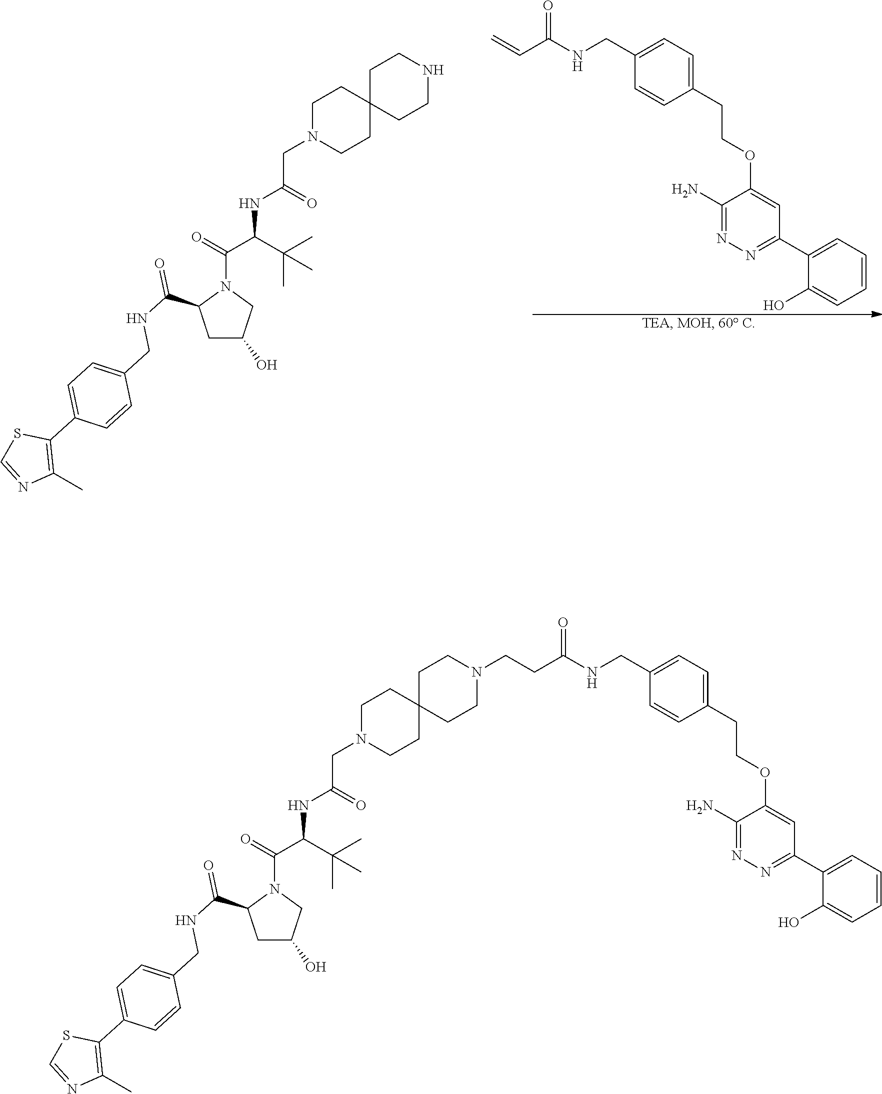 [SMILES: Cc1ncsc1-c1ccc(CNC(=O)[C@@H]2C[C@@H](O)CN2C(=O)[C@@H](NC(=O)CN2CCC3(CCNCC3)CC2)C(C)(C)C)cc1 ; C=CC(=O)NCc1ccc(CCOc2cc(-c3ccccc3O)nnc2N)cc1 ; "TEA, MOH, 60° C." ; Cc1ncsc1-c1ccc(CNC(=O)[C@@H]2C[C@@H](O)CN2C(=O)[C@@H](NC(=O)CN2CCC3(CCN(CCC(=O)NCc4ccc(CCOc5cc(-c6ccccc6O)nnc5N)cc4)CC3)CC2)C(C)(C)C)cc1]

### Step 1: Preparation of tert-butyl 9-(2-ethoxy-2-oxoethyl)-3,9-diazaspiro[5.5]undecane-3-carboxylate

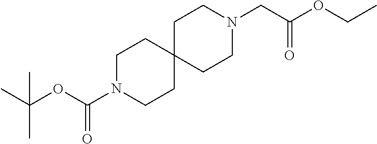 [SMILES: CCOC(=O)CN1CCC2(CC1)CCN(C(=O)OC(C)(C)C)CC2]

To a stirred mixture of tert-butyl 3,9-diazaspiro[5.5]undecane-3-carboxylate (200.00 mg, 0.786 mmol, 1.00 equiv) and ethyl bromoacetate (131.30 mg, 0.786 mmol, 1.00 equiv) in DMF (6.00 mL) was added K₂CO₃ (217.33 mg, 1.572 mmol, 2.00 equiv) at room temperature. The resulting mixture was stirred for 3 h at 60 degrees C. The mixture was allowed to cool down to room temperature and water (100.00 ml) was added. Following extraction with EtOAc (3×100 mL), the combined organic layers were washed with brine (2×30 mL), and dried over anhydrous Na₂SO₄. After filtration, the filtrate was concentrated under reduced pressure to afford the title compound (310 mg, crude) as a light yellow oil. The crude product was used in the next step directly without further purification. LCMS (ESI) m/z: [M+H]⁺=340.46

### Step 2: Preparation of 2-(9-(tert-butoxycarbonyl)-3,9-diazaspiro[5.5]undecan-3-yl)acetic acid

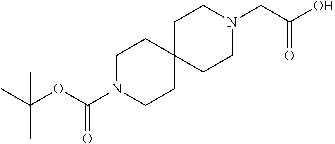 [SMILES: CC(C)(C)OC(=O)N1CCC2(CCN(CC(=O)O)CC2)CC1]

To a stirred solution of tert-butyl 9-(2-ethoxy-2-oxoethyl)-3,9-diazaspiro[5.5]undecane-3-carboxylate (312.00 mg, 0.916 mmol, 1.00 equiv) and LiOH (219.46 mg, 9.164 mmol, 10.00 equiv) in MeOH (5.00 mL) was added H₂O (2.50 mL) at room temperature. The resulting mixture was stirred for 3 h. The mixture was acidified to pH 6 with 0.5M HCl. The resulting mixture was filtered, the filter cake was triturated with EA (3×10 mL) to afford the title compound (132 mg, 46.11%) as a white solid. LCMS (ESI) m/z: [M+H]⁺=312.41.

### Step 3: Preparation of tert-butyl 9-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethyl)-3,9-diazaspiro[5.5]undecane-3-carboxylate

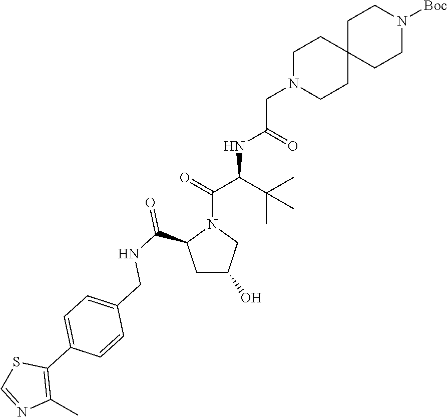 [SMILES: Cc1ncsc1-c1ccc(CNC(=O)[C@@H]2C[C@@H](O)CN2C(=O)[C@@H](NC(=O)CN2CCC3(CC2)CCN(C(=O)OC(C)(C)C)CC3)C(C)(C)C)cc1]

To a stirred mixture 2-(9-(tert-butoxycarbonyl)-3,9-diazaspiro[5.5]undecan-3-yl)acetic acid (100.00 mg, 0.320 mmol, 1.00 equiv) and (2S,4R)-1-[(2S)-2-amino-3,3-dimethylbutanoyl]-4-hydroxy-N-[[4-(4-methyl-1,3-thiazol-5-yl)phenyl]methyl]pyrrolidine-2-carboxamide (137.82 mg, 0.320 mmol, 1.00 equiv) in DMF (4.00 mL) were added HATU (146.05 mg, 0.384 mmol, 1.20 equiv) and DIEA (206.85 mg, 1.600 mmol, 5.00 equiv) at room temperature. The resulting mixture was stirred for 2 h at room temperature. The resulting mixture was filtered, the filtrate concentrated and purified by reverse flash chromatography (eluting with 0-100% acetonitrile in water over 20 min.) to afford the title compound (75 mg, 17.45%) as a white solid. LCMS (ESI) m/z: [M+H]⁺=724.96.

### Step 4: Preparation of (2S,4R)-1-((S)-2-(2-(3,9-diazaspiro[5.5]undecan-3-yl)acetamido)-3,3-dimethylbutanoyl)-4-hydroxy-N-(4-(4-methylthiazol-5-yl)benzyl)pyrrolidine-2-carboxamide

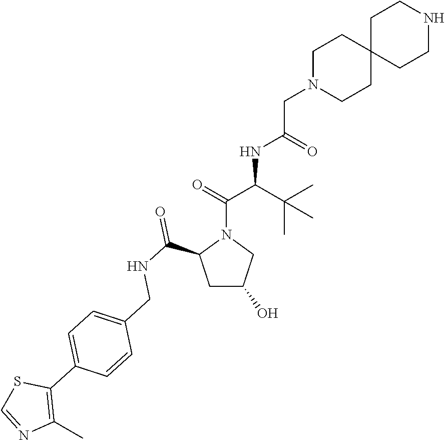 [SMILES: Cc1ncsc1-c1ccc(CNC(=O)[C@@H]2C[C@@H](O)CN2C(=O)[C@@H](NC(=O)CN2CCC3(CCNCC3)CC2)C(C)(C)C)cc1]

To a stirred solution of tert-butyl 9-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethyl)-3,9-diazaspiro[5.5]undecane-3-carboxylate (75.00 mg, 0.103 mmol, 1.00 equiv) in DCM (3.00 mL) was added TFA (1.00 mL, 13.463 mmol, 130.14 equiv) at room temperature. The resulting mixture was stirred for 3 h at room temperature. The resulting mixture was concentrated under reduced pressure. The residue was dissolved in DMF (2.00 mL) and was purified by reverse flash chromatography (eluting with 0-100% acetonitrile in water over 20 min.) to afford the title compound (43 mg, 46.56%) as a white solid. LCMS (ESI) m/z: [M+H]⁺=624.85

### Step 5: Preparation of (2S,4R)-1-((S)-2-(2-(9-(3-((4-(2-((3-amino-6-(2-hydroxyphenyl)pyridazin-4-yl)oxy)ethyl)benzyl)amino)-3-oxopropyl)-3,9-diazaspiro[5.5]undecan-3-yl)acetamido)-3,3-dimethylbutanoyl)-4-hydroxy-N-(4-(4-methylthiazol-5-yl)benzyl)pyrrolidine-2-carboxamide (Compound 266)

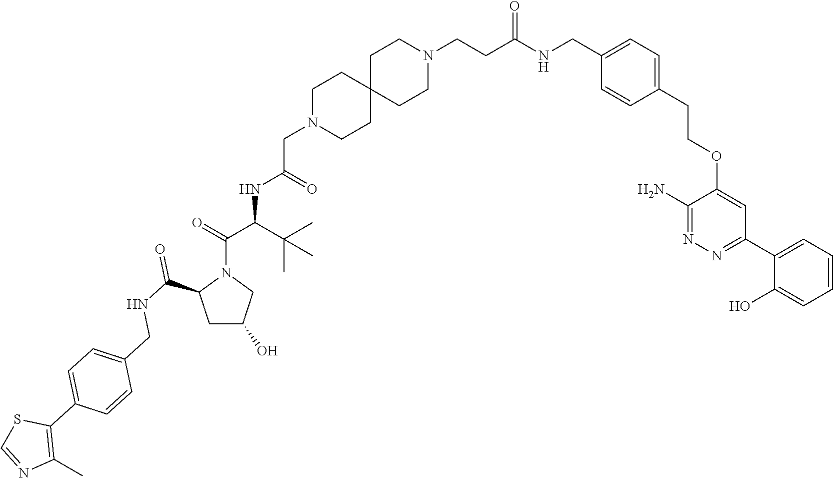 [SMILES: Cc1ncsc1-c1ccc(CNC(=O)[C@@H]2C[C@@H](O)CN2C(=O)[C@@H](NC(=O)CN2CCC3(CCN(CCC(=O)NCc4ccc(CCOc5cc(-c6ccccc6O)nnc5N)cc4)CC3)CC2)C(C)(C)C)cc1]

To a stirred mixture of (2S,4R)-1-((S)-2-(2-(3,9-diazaspiro[5.5]undecan-3-yl)acetamido)-3,3-dimethylbutanoyl)-4-hydroxy-N-(4-(4-methylthiazol-5-yl)benzyl)pyrrolidine-2-carboxamide (25.00 mg, 0.040 mmol, 1.00 equiv) and N-(4-(2-((3-amino-6-(2-hydroxyphenyl)pyridazin-4-yl)oxy)ethyl)benzyl)acrylamide (15.62 mg, 0.040 mmol, 1.00 equiv) in MeOH (1.00 mL) was added TEA (12.15 mg, 0.120 mmol, 3.00 equiv) at room temperature. The resulting mixture was stirred overnight at 60 degrees C. The resulting mixture was filtered and the filtrate was purified by preparative HPLC (XBridge Prep C18 OBD column, eluting with 35-50% acetonitrile in 10 mM aqueous ammonium carbonate), to provide the title compound (7.2 mg, 17.42%) as a white solid. ¹H NMR (400 MHz, DMSO-d6) δ 14.37 (s, 1H), 8.98 (s, 1H), 8.60 (t, J=6.0 Hz, 1H), 8.41 (t, J=5.9 Hz, 1H), 7.94 (dd, J=8.0, 1.6 Hz, 1H), 7.79 (d, J=9.7 Hz, 1H), 7.59 (s, 1H), 7.41 (q, J=8.3 Hz, 4H), 7.34 (d, J=8.0 Hz, 2H), 7.26-7.18 (m, 3H), 6.86 (t, J=7.9 Hz, 2H), 6.49 (s, 2H), 5.14 (d, J=3.4 Hz, 1H), 4.52-4.33 (m, 6H), 4.26 (dd, J=13.2, 5.7 Hz, 3H), 3.67-3.56 (m, 2H), 3.12 (t, J=6.8 Hz, 2H), 3.00-2.84 (m, 2H), 2.60-2.53 (m, 2H), 2.46-2.42 (m, 4H), 2.42-2.35 (m, 3H), 2.32-2.23 (m, 6H), 2.09-2.01 (m, 1H), 1.94-1.85 (m, 1H), 1.48-1.31 (m, 8H), 0.93 (s, 9H). LCMS (ESI) m/z: [M+H]⁺=1015.65.

The following compounds in Table E8 were prepared using procedures similar to those used for the preparation of compound 266.

TABLE E8

| | | LCMS | |
|---|---|---|---|
| | | (ESI) | |
| No. | Name | m/z | ¹H NMR |
| 235 | (2S,4R)-1-((2S)-2-(3-(5-(3-((4- | 987.48 | ¹H NMR (400 MHz, DMSO-d⁶) δ 14.38 (s, 1H), |
| | (2-((3-amino-6-(2- | | 8.96 (s, 1H), 8.58 (t, J = 6.1 Hz, 1H), 8.42 (d, |
| | hydroxyphenyl)pyridazin-4- | | J = 9.4 Hz, 1H), 8.19 (s, 1H), 7.94 (dd, J = 8.1, |
| | yl)oxy)ethyl)benzyl)amino)-3- | | 1.6 Hz, 1H), 7.59 (s, 1H), 7.48-7.31 (m, 6H), |
| | oxopropyl)hexahydropyrrolo[3,4- | | 7.27-7.16 (m, 3H), 6.86 (t, J = 7.8 Hz, 2H), |
| | c]pyrrol-2(1H)- | | 6.50 (s, 2H), 5.13 (d, J = 3.5 Hz, 1H), 4.54 (d, |
| | yl)propanamido)-3,3- | | J = 9.4 Hz, 1H), 4.47-4.38 (m, 4H), 4.35 (s, |
| | dimethylbutanoyl)-4-hydroxy-N- | | 1H), 4.29-4.18 (m, 3H), 3.69-3.59 (m, 2H), |
| | (4-(4-methylthiazol-5- | | 3.31 (s, 3H), 3.12 (t, J = 6.8 Hz, 2H), 2.93 (s, |
| | yl)benzyl)pyrrolidine-2- | | 2H), 2.51 (s, 2H), 2.44 (s, 3H), 2.40 (s, 4H), |
| | carboxamide | | 2.30 (s, 2H), 2.35-2.21 (m, 1H), 2.04 (dd, J = |
| | | | 19.9, 8.1 Hz, 1H), 1.90 (ddd, J = 12.8, 8.6, 4.5 |
| | | | Hz, 1H), 0.94 (s, 9H). |
| 236 | (2S,4R)-1-((S)-2-(3-(4-((1-(3- | 1059.54 | ¹H NMR (400 MHz, DMSO-d6) δ 14.38 (s, 1H), |
| | ((4-(2-((3-amino-6-(2- | | 8.97 (s, 1H), 8.72 (s, 1H), 8.61 (s, 1H), 8.41 (s, |
| | hydroxyphenyl)pyridazin-4- | | 1H), 7.94 (d, J = 7.9 Hz, 1H), 7.59 (s, 1H), |
| | yl)oxy)ethyl)benzyl)amino)-3- | | 7.47-7.31 (m, 6H), 7.27-7.19 (m, 3H), 6.87 (t, |
| | oxopropyl)piperidin-4- | | J = 8.0 Hz, 2H), 6.50 (s, 2H), 5.14 (d, J = 3.4 |
| | yl)oxy)piperidin-1- | | Hz, 1H), 4.55 (d, J = 9.4 Hz, 1H), 4.50-4.39 |
| | yl)propanamido)-3,3- | | (m, 3H), 4.35 (s, 1H), 4.23 (dd, J = 12.6, 5.5 |
| | dimethylbutanoyl)-4-hydroxy-N- | | Hz, 3H), 3.67 (dd, J = 10.5, 3.9 Hz, 3H), 3.61 |
| | (4-(4-methylthiazol-5- | | (d, J = 10.7 Hz, 4H), 3.12 (t, J = 6.8 Hz, 3H), |
| | yl)benzyl)pyrrolidine-2- | | 2.67 (dd, J = 3.7, 1.9 Hz, 3H), 2.44 (s, 5H), |
| | carboxamide | | 2.29 (s, 3H), 2.04 (t, J = 10.5 Hz, 4H), 1.95- |
| | | | 1.85 (m, 2H), 1.80 (s, 2H), 1.69 (s, 2H), 1.48 |
| | | | (s, 2H), 1.34 (s, 2H), 0.94 (s, 9H). |
| 267 | (2S,4R)-1-((S)-2-(2-(9-(3-((4-(2- | 1029.75 | ¹H NMR (400 MHz, DMSO-d6) δ 14.37 (s, 1H), |
| | ((3-amino-6-(2- | | 8.98 (s, 1H), 8.47-8.37 (m, 2H), 7.97-7.90 |
| | hydroxyphenyl)pyridazin-4- | | (m, 1H), 7.72 (d, J = 9.7 Hz, 1H), 7.60 (s, 1H), |
| | yl)oxy)ethyl)benzyl)amino)-3- | | 7.46-7.41 (m, 2H), 7.39-7.31 (m, 4H), 7.26- |
| | oxopropyl)-3,9- | | 7.19 (m, 3H), 6.86 (t, J = 7.6 Hz, 2H), 6.49 (s, |
| | diazaspiro[5.5]undecan-3- | | 2H), 5.12 (d, J = 3.4 Hz, 1H), 4.89 (q, J = 7.1 |
| | yl)acetamido)-3,3- | | Hz, 1H), 4.52-4.38 (m, 4H), 4.30-4.21 (m, |
| | dimethylbutanoyl)-4-hydroxy-N- | | 3H), 3.62-3.52 (m, 2H), 3.12 (t, J = 6.8 Hz, |
| | ((S)-1-(4-(4-methylthiazol-5- | | 2H), 2.99-2.82 (m, 2H), 2.58-2.54 (m, 2H), |
| | yl)phenyl)ethyl)pyrrolidine-2- | | 2.47-2.43 (m, 4H), 2.43-2.33 (m, 6H), 2.32- |
| | carboxamide | | 2.25 (m, 3H), 2.09-2.01 (m, 1H), 1.79- |
| | | | 1.70 (m, 1H), 1.50-1.29 (m, 11H), 0.92 (s, |
| | | | 9H). |
| 258 | (2S,4R)-1-((S)-2-(2-(4-((1-(3- | 1059.94 | ¹H NMR (300 MHz, DMSO-d6) δ 14.39 (s, 1H), |
| | ((4-(2-((3-amino-6-(2- | | 8.99 (s, 1H), 8.49-8.35 (m, 2H), 8.00-7.90 |
| | hydroxyphenyl)pyridazin-4- | | (m, 1H), 7.77 (d, J = 9.7 Hz, 1H), 7.60 (s, 1H), |
| | yl)oxy)ethyl)benzyl)amino)-3- | | 7.44 (d, J = 8.3 Hz, 2H), 7.40-7.31 (m, 4H), |
| | oxopropyl)piperidin-4- | | 7.23 (dd, J = 8.4, 3.6 Hz, 3H), 6.93-6.81 (m, |
| | yl)oxy)piperidin-1- | | 2H), 6.50 (s, 2H), 5.13 (d, J = 3.4 Hz, 1H), 4.90 |
| | yl)acetamido)-3,3- | | (t, J = 7.2 Hz, 1H), 4.53-4.38 (m, 4H), 4.25 |
| | dimethylbutanoyl)-4-hydroxy-N- | | (d, J = 6.0 Hz, 3H), 3.58 (s, 2H), 3.41 (s, 2H), |
| | ((S)-1-(4-(4-methylthiazol-5- | | 3.13 (t, J = 6.8 Hz, 2H), 3.01 (d, J = 16.3 Hz, |
| | yl)phenyl)ethyl)pyrrolidine-2- | | 2H), 2.87 (d, J = 16.1 Hz, 3H), 2.68 (s, 1H), |
| | carboxamide | | 2.46 (s, 3H), 2.28 (dd, J = 4.8, 2.4 Hz, 5H), |
| | | | 2.07 (s, 3H), 1.76(m, 5H) 1.42 (s, 3H), 1.37 (s, |
| | | | 4H), 1.24 (s, 1H), 0.93 (s, 9H). |

### Example 18. Degradation of BRM and BRG1 by Compounds of the Invention

This example demonstrates the ability of the compounds of the disclosure to degrade a HiBit-BRM or HiBit-BRG1 fusion protein in a cell-based degradation assay.

Procedure: A stable HeLa cell line expressing HiBiT-BRM was generated. On day 0, 5000 cells were seeded in 40 μL of media into each well of 384-well cell culture plates. On day 1, cells were treated with 120 nL DMSO or 120 nL of 3-fold serially DMSO-diluted compounds (10 points in duplicate with 30 μM as final top dose). Subsequently plates were incubated for 24 h in a standard tissue culture incubator and equilibrated at room temperature for 15 minutes. Nano-Glo HiBiT Lytic Detection System (Promega N3050) reagent was freshly prepared and 20 ul was added to each well. Upon addition of this LgBit-containing reagent, the HiBiT and LgBiT proteins associate to form the luminescent NanoBiT luciferase. The plates were shaken for 10 minutes at room temperature and the bioluminescence read using an EnVision plate reader (PerkinElmer).

For measurement of BRG1 degradation, a stable HeLa cell line expressing HiBit-BRG1 and LgBit was generated. The same protocol as above was then followed.

The degradation % was calculated using the following formula: % degradation=100%−100%×(Lum_{Sample}−Lum_{LC})/(Lum_{HC}−Lum_{LC}). DMSO treated cells are employed as High Control (HC) and 2 μM of a known BRM/BRG1 degrader standard treated cells are employed as Low Control (LC). The data was fit to a four parameter, non-linear curve fit to calculate IC₅₀ (μM) values as shown in Table 3.

Results: As shown in Table 3 below, the compounds of the invention degraded both BRM and BRG1.

TABLE 3

| | | BRM HiBit | | BRG1 HiBit |
|---|---|---|---|---|
| | BRM HiBit | Degradation | BRG1 HiBit | Degradation |
| Compound | Degradation | Maximum | Degradation | Maximum |
| No. | IC₅₀ (nM) | (%) | IC₅₀ (nM) | (%) |
| 1 | ++ | B | + | C |
| 2 | + | C | + | C |
| 3 | + | C | + | C |
| 4 | ++ | B | + | C |
| 5 | + | C | + | C |
| 6 | + | C | + | C |
| 7 | + | C | + | C |
| 8 | + | B | + | C |
| 9 | ++ | A | + | C |
| 10 | + | C | + | C |
| 11 | + | C | + | C |
| 12 | + | C | + | C |
| 13 | + | C | + | C |
| 14 | + | C | + | C |
| 15 | + | C | + | C |
| 16 | + | C | + | C |
| 17 | + | C | + | C |
| 18 | + | C | + | C |
| 19 | + | C | + | C |
| 20 | +++ | A | ++ | A |
| 21 | ++ | B | + | C |
| 22 | + | C | + | C |
| 23 | + | C | + | C |
| 24 | + | C | + | C |
| 25 | + | C | + | C |
| 26 | ++ | A | + | C |
| 27 | + | A | + | C |
| 28 | ++ | A | + | C |
| 29 | + | B | + | C |
| 30 | ++ | A | + | C |
| 31 | +++ | B | + | C |
| 32 | +++ | A | + | C |
| 33 | ++ | A | ++ | B |
| 34 | + | B | + | C |
| 35 | +++ | A | + | C |
| 36 | +++ | B | + | C |
| 37 | ++ | B | + | C |
| 38 | + | B | + | C |
| 39 | + | C | + | C |
| 40 | ++ | A | + | C |
| 41 | + | C | + | C |
| 42 | + | C | + | C |
| 43 | + | C | + | C |
| 44 | ++ | B | + | C |
| 45 | + | C | + | C |
| 46 | + | C | + | C |
| 47 | ++ | B | + | C |
| 48 | + | C | + | C |
| 49 | + | C | + | C |
| 50 | + | C | + | C |
| 51 | + | C | + | C |
| 52 | + | C | + | C |
| 53 | + | C | + | C |
| 54 | + | C | + | C |
| 55 | + | C | + | C |
| 56 | + | C | + | C |
| 57 | ++ | B | + | C |
| 58 | + | C | + | C |
| 59 | + | B | + | C |
| 60 | + | B | + | C |
| 61 | ++ | B | + | C |
| 62 | + | C | ++ | B |
| 63 | ++ | B | + | C |
| 64 | + | C | + | C |
| 65 | + | C | + | C |
| 66 | + | C | + | C |
| 67 | ++ | A | + | C |
| 68 | NT | NT | NT | NT |
| 69 | + | C | + | C |
| 70 | + | C | + | C |
| 71 | + | C | + | C |
| 72 | + | C | + | C |
| 73 | + | C | + | C |
| 74 | + | C | + | C |
| 75 | + | C | + | C |
| 76 | + | C | + | C |
| 77 | + | C | + | C |
| 78 | + | C | + | C |
| 79 | + | C | + | C |
| 80 | + | C | + | C |
| 81 | + | C | + | C |
| 82 | + | C | + | C |
| 83 | ++ | B | + | C |
| 84 | + | C | + | C |
| 85 | NT | NT | NT | NT |
| 86 | + | C | + | C |
| 87 | + | B | + | B |
| 88 | + | C | + | C |
| 89 | + | C | + | C |
| 90 | + | C | + | C |
| 91 | + | C | + | C |
| 92 | + | C | + | C |
| 93 | + | C | + | C |
| 94 | + | A | + | C |
| 95 | + | C | + | C |
| 96 | + | C | + | C |
| 97 | + | C | + | C |
| 98 | + | C | + | C |
| 99 | + | C | + | C |
| 100 | + | C | + | C |
| 101 | + | B | + | C |
| 102 | ++ | A | + | C |
| 103 | + | C | + | C |
| 104 | + | C | + | C |
| 105 | + | C | + | C |
| 106 | + | C | + | C |
| 107 | + | C | + | B |
| 108 | +++ | B | + | B |
| 109 | + | C | + | C |
| 110 | +++ | A | ++ | B |
| 111 | + | C | + | C |
| 112 | +++ | B | + | B |
| 113 | +++ | A | + | C |
| 114 | +++ | B | + | B |
| 115 | + | C | + | C |
| 116 | + | C | + | C |
| 117 | + | B | + | A |
| 118 | ++ | B | + | C |
| 119 | + | C | + | B |
| 120 | ++ | B | + | B |
| 121 | + | C | + | C |
| 122 | ++ | B | + | B |
| 123 | +++ | B | + | C |
| 124 | + | C | + | C |
| 125 | + | C | + | B |
| 126 | + | C | + | C |
| 127 | + | C | + | C |
| 128 | + | C | + | C |
| 129 | + | C | + | C |
| 130 | + | C | + | C |
| 131 | ++ | A | ++ | B |
| 132 | ++ | A | + | B |
| 133 | ++ | B | + | C |
| 134 | ++ | B | + | C |
| 135 | + | C | + | C |
| 136 | ++ | B | + | C |
| 137 | + | C | + | C |
| 138 | ++ | A | + | C |
| 139 | ++ | A | + | B |
| 140 | ++ | A | + | C |
| 141 | + | C | + | C |
| 142 | + | C | + | C |
| 143 | + | C | + | C |
| 144 | ++ | A | + | C |
| 145 | + | C | + | C |
| 146 | + | C | + | C |
| 147 | + | C | + | C |
| 148 | + | C | + | C |
| 149 | ++ | A | ++ | B |
| 150 | + | C | + | C |
| 151 | + | C | + | B |
| 152 | ++ | A | + | C |
| 153 | + | C | + | C |
| 154 | + | C | + | C |
| 155 | ++ | A | + | C |
| 156 | +++ | B | ++++ | B |
| 158 | ++ | B | ++ | B |
| 159 | +++ | A | + | C |
| 160 | + | B | + | B |
| 161 | NT | | NT | |
| 162 | + | B | + | C |
| 163 | + | B | + | C |
| 164 | + | B | + | C |
| 165 | + | A | + | C |
| 166 | + | C | + | C |
| 167 | + | B | + | C |
| 168 | + | C | + | C |
| 169 | ++ | B | + | C |
| 170 | + | C | + | C |
| 171 | ++ | A | + | B |
| 172 | +++ | A | ++ | B |
| 173 | ++ | B | + | C |
| 174 | +++ | A | ++ | A |
| 175 | +++ | A | ++ | B |
| 176 | +++ | A | ++ | A |
| 177 | ++ | A | + | A |
| 178 | ++ | A | ++ | A |
| 179 | + | C | + | C |
| 180 | + | B | + | C |
| 181 | + | C | + | C |
| 182 | + | C | + | C |
| 183 | + | C | + | C |
| 184 | + | B | + | C |
| 185 | + | C | + | C |
| 186 | + | C | + | C |
| 187 | + | C | + | C |
| 188 | + | C | + | C |
| 189 | + | C | + | C |
| 190 | +++ | A | ++ | A |
| 191 | ++ | A | + | A |
| 192 | ++ | A | + | B |
| 193 | ++ | B | + | C |
| 194 | +++ | A | ++ | A |
| 195 | +++ | A | ++ | A |
| 196 | + | C | + | C |
| 197 | +++ | A | + | C |
| 198 | +++ | A | + | C |
| 199 | + | C | + | C |
| 200 | + | C | + | C |
| 201 | + | B | + | C |
| 202 | + | B | + | C |
| 203 | + | B | + | C |
| 204 | + | B | + | C |
| 205 | ++++ | A | ++ | A |
| 206 | +++ | A | ++ | A |
| 207 | +++ | A | + | C |
| 208 | +++ | A | ++ | A |
| 209 | +++ | A | ++ | A |
| 210 | ++++ | A | ++ | B |
| 211 | +++ | A | + | B |
| 212 | +++ | A | + | C |
| 213 | +++ | A | ++ | A |
| 214 | +++ | A | + | C |
| 215 | +++ | A | + | C |
| 216 | + | C | + | C |
| 217 | +++ | A | + | C |
| 218 | ++ | B | + | C |
| 219 | + | B | + | C |
| 220 | ++++ | A | +++ | A |
| 221 | +++ | A | ++++ | A |
| 222 | ++ | A | ++ | A |
| 223 | ++ | A | + | C |
| 224 | + | C | + | C |
| 225 | ++ | A | ++ | A |
| 226 | + | B | + | C |
| 227 | +++ | A | ++ | A |
| 228 | ++ | A | ++ | A |
| 229 | + | B | + | C |
| 230 | ++ | A | + | A |
| 231 | + | C | + | C |
| 232 | ++ | A | + | C |
| 233 | +++ | A | ++ | A |
| 234 | ++ | A | + | C |
| 235 | + | C | + | C |
| 236 | + | B | + | C |
| 237 | +++ | A | ++ | B |
| 238 | + | C | + | C |
| 239 | ++ | A | + | C |
| 240 | ++ | A | ++ | A |
| 241 | + | C | + | C |
| 242 | ++ | A | + | C |
| 243 | ++++ | A | +++ | A |
| 244 | + | A | + | C |
| 245 | + | B | + | C |
| 246 | +++ | A | + | C |
| 247 | +++ | A | + | C |
| 248 | +++ | A | + | C |
| 249 | + | A | + | C |
| 250 | + | B | + | C |
| 251 | ++++ | A | ++++ | A |
| 252 | + | C | + | C |
| 253 | ++++ | A | + | C |
| 254 | +++ | B | + | C |
| 255 | +++ | A | + | C |
| 256 | + | C | + | C |
| 257 | +++ | A | + | C |
| 258 | ++ | A | + | B |
| 259 | + | C | + | C |
| 260 | + | C | + | C |
| 261 | +++ | A | + | B |
| 262 | + | B | + | C |
| 263 | +++ | A | + | C |
| 264 | +++ | A | ++ | B |
| 265 | + | B | + | C |
| 266 | + | A | + | C |
| 267 | + | A | + | C |

“+” indicates inhibitory effect of ≥1000 nM;
“++” indicates inhibitory effect of ≥100 nM;
“+++” indicates inhibitory effect of ≥10 nM;
“++++” indicates inhibitory effect of <10 nM;
“NT” indicates not tested;
“A” indicates maximum degradation ≥75%;
“B” indicates maximum degradation ≥50%;
and “C” indicates maximum degradation <50%

### OTHER EMBODIMENTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

1. A compound having the structure of Formula I: 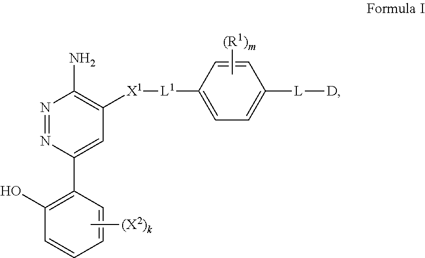
wherein
X¹ is O or NR²;
each X² is independently a halogen;
k is 0, 1, 2, 3, or 4;
m is 0, 1, 2, 3, or 4;
R¹ is halo or optionally substituted C₁-C₆ alkyl;
R² is H or optionally substituted C₁-C₆ alkyl;
L¹ is optionally substituted C₁-C₆ alkylene;
L is a linker comprising the structure of 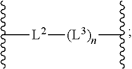
n is 0, 1, 2, or 3;
L² is optionally substituted C₁-C₆ alkylene, optionally substituted C₁-C₂₀ heteroalkylene, or optionally substituted C₂-C₉ heterocyclylene;
each L³ is, independently, —O—, optionally substituted C₁-C₂₀ heteroalkylene, optionally substituted C₃-C₁₀ carbocyclylene, optionally substituted C₃-C₁₀ carbocyclylene-C₁-C₂₀ alkylene, optionally substituted C₂-C₉ heterocyclylene, or optionally substituted C₂-C₉ heterocyclylene-C₁-C₂₀ alkylene; and
D is a degradation moiety,
or a pharmaceutically acceptable salt thereof.

2. The compound of claim 1, wherein m is 0.

3. The compound of claim 1, wherein X¹ is O.

4. The compound of claim 1, wherein X¹ is NR².

5. The compound of claim 4, wherein R² is optionally substituted C₁-C₆ alkyl.

6. (canceled)

7. The compound of claim 1, wherein L¹ is

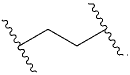 [SMILES: *CC*]

8. (canceled)

9. (canceled)

10. The compound of claim 1, wherein L² is

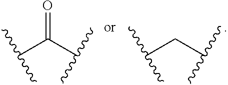

11. (canceled)

12. The compound of claim 1, wherein L² is

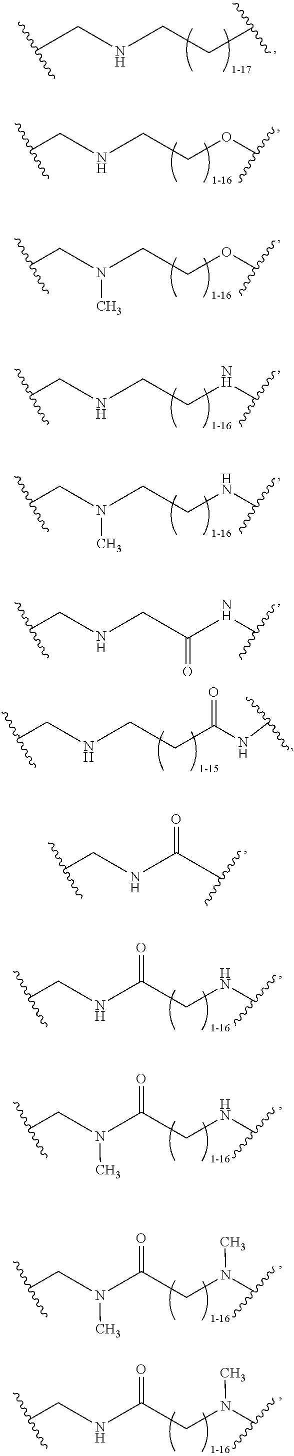

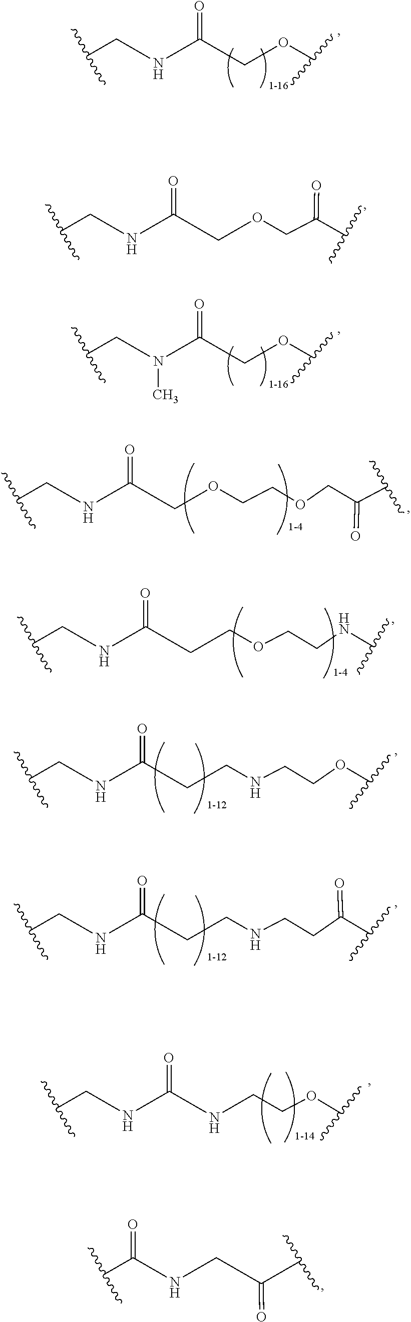

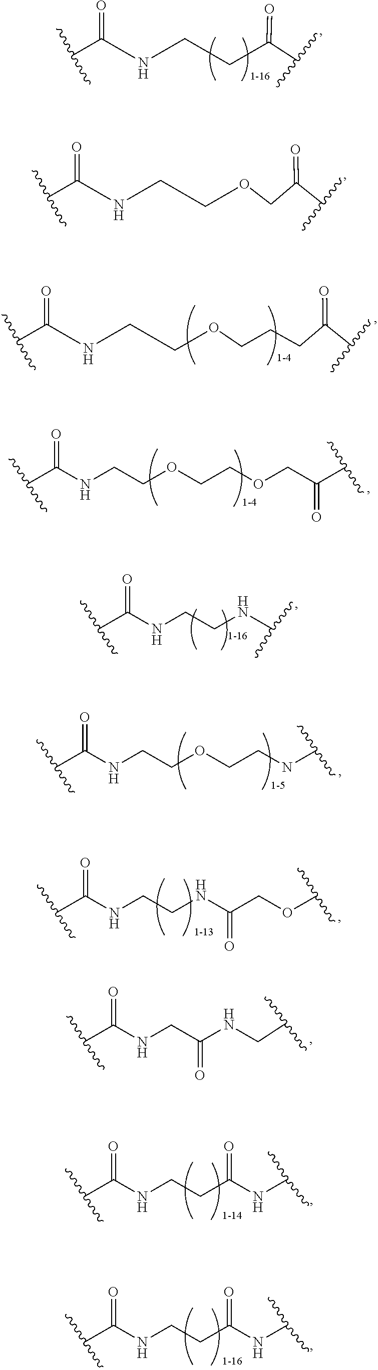

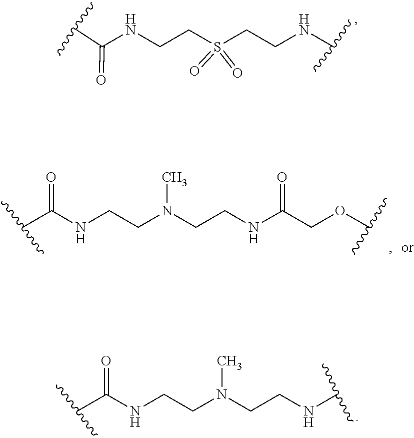 [SMILES: *NCCS(=O)(=O)CCNC(*)=O ; *OCC(=O)NCCN(C)CCNC(*)=O ; *NCCN(C)CCNC(*)=O]

13. The compound of claim 1, wherein n is 1, 2, or 3.

14.-16. (canceled)

17. The compound of claim 1, wherein each L³ is, independently, optionally substituted C₃-C₁₀ carbocyclylene-C₁-C₆ alkylene, optionally substituted C₂-C₉ heterocyclylene, or optionally substituted C₂-C₉ heterocyclylene-C₁-C₆ alkylene.

18. The compound of claim 1, wherein each L³ is, independently,

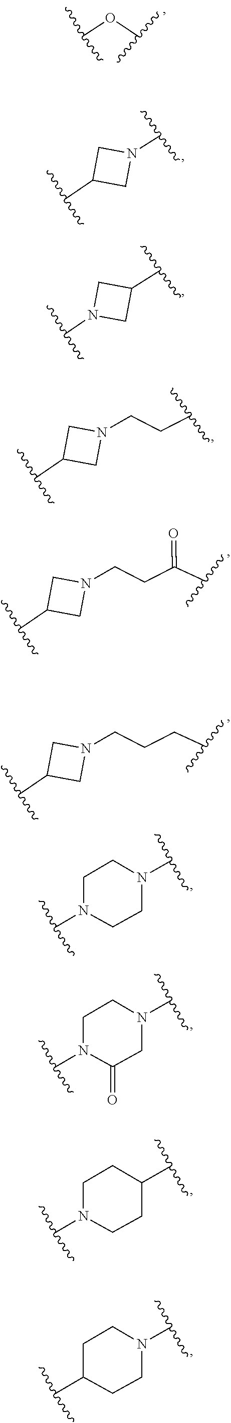

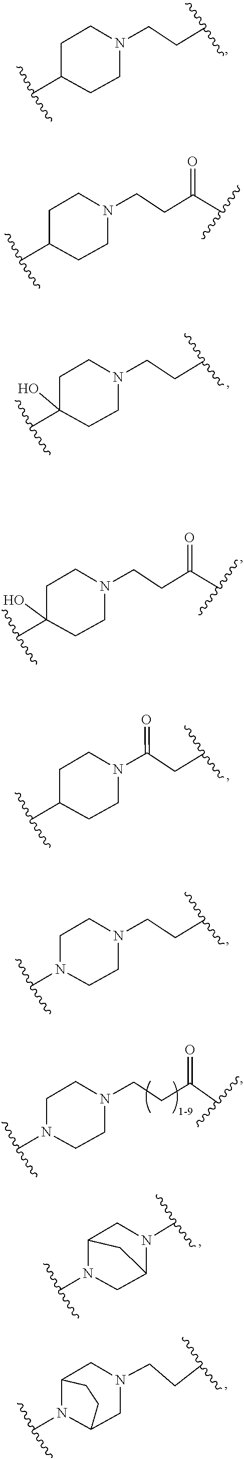

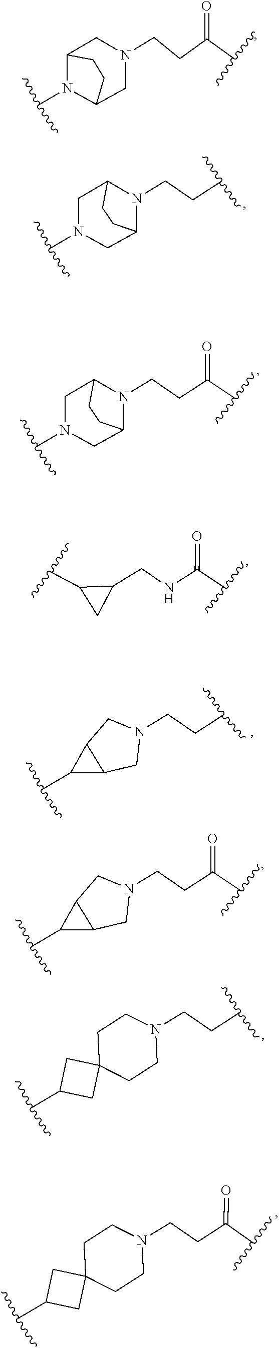

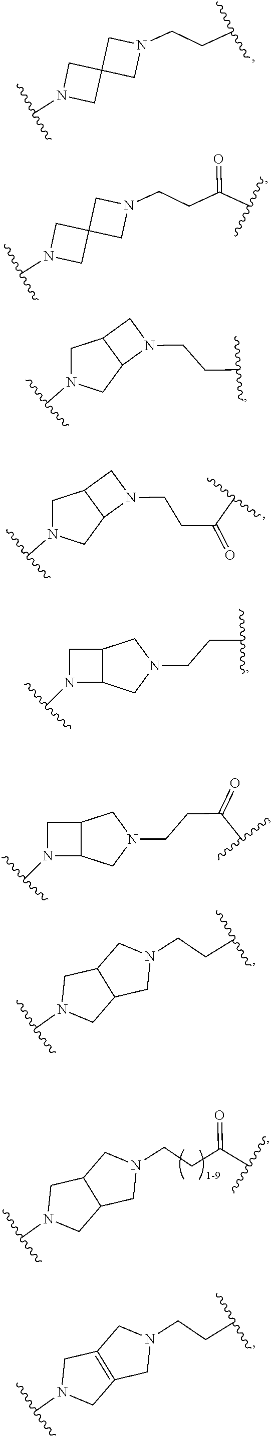

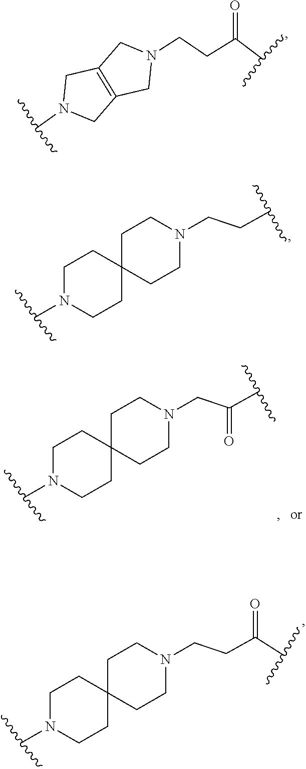 [SMILES: *C(=O)CCN1CC2=C(CN(*)C2)C1 ; *CCN1CCC2(CCN(*)CC2)CC1 ; *C(=O)CN1CCC2(CCN(*)CC2)CC1 ; *C(=O)CCN1CCC2(CCN(*)CC2)CC1]

19. The compound of claim 1, wherein n is 0.

20. The compound of claim 1, wherein k is 0, 1, or 2, and when k is 1 or 2, each X² is independently fluorine or chlorine.

21.-40. (canceled)

41. The compound of claim 1, wherein D is the degradation moiety is a ubiquitin ligase binding moiety.

42. The compound of claim 41, wherein the ubiquitin ligase binding moiety comprises a Cereblon ligand, an IAP (Inhibitors of Apoptosis) ligand, a mouse double minute 2 homolog (MDM2), or a von Hippel-Lindau ligand, or derivatives or analogs thereof.

43. The compound of claim 41, wherein degradation moiety comprises the structure of Formula A: 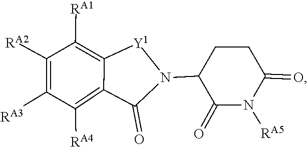
wherein
Y¹ is 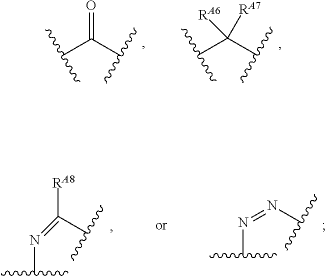
R^{A5} is H, optionally substituted C₁-C₆ alkyl, or optionally substituted C₁-C₆ heteroalkyl;
R^{A6} is H or optionally substituted C₁-C₆ alkyl; and R^{A7} is H or optionally substituted C₁-C₆ alkyl; or R^{A6} and R^{A7}, together with the carbon atom to which each is bound, combine to form optionally substituted C₃-C₆ carbocyclyl or optionally substituted C₂-C₅ heterocyclyl; or R^{A6} and R^{A7}, together with the carbon atom to which each is bound, combine to form optionally substituted C₃-C₆ carbocyclyl or optionally substituted C₂-C₅ heterocyclyl;
R^{A8} is H, optionally substituted C₁-C₆ alkyl, or optionally substituted C₁-C₆ heteroalkyl;
each of R^{A1}, R^{A2}, R^{A3}, and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form 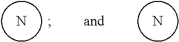
is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A²,
where one of R^{A1}, R^{A2}, R^{A3}, and R^{A4} is A², or 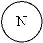
is substituted with A²; and
A² is a bond between the degradation moiety and the linker,
or a pharmaceutically acceptable salt thereof.

44. The compound of claim 43, wherein R^{A5} is H or

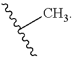

45. (canceled)

46. The compound of claim 43, wherein each of R^{A1}, R^{A2}, R^{A3}, and R^{A4} is, independently, H or A².

47. The compound of claim 46, wherein
R^{A1} is A² and each of R^{A2}, R^{A3}, and R^{A4} is H, or
R^{A2} is A² and each of R^{A1}, R^{A3}, and R^{A4} is H, or
R^{A3} is A² and each of R^{A1}, R^{A2}, and R^{A4} is H, or
R^{A4} is A² and each of R^{A1}, R^{A2}, and R^{A3} is H.

48.-50. (canceled)

51. The compound of claim 43, wherein Y¹ is

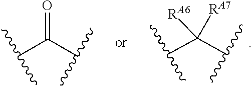

52. The compound of claim 47, wherein R^{A6} is H.

53. The compound of claim 47, wherein R^{A7} is H.

54. The compound of claim 47, wherein Y¹ is

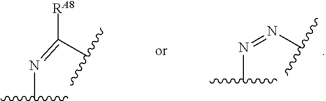

55. The compound of claim 54, wherein R^{A8} is H or optionally substituted C₁-C₆ alkyl.

56. The compound of claim 55, wherein R^{A8} is H or

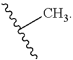

57. The compound of claim 56, wherein R^{A8} is

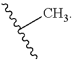

58. The compound of claim 43, wherein the degradation moiety comprises the structure of Formula A2, Formula A4, Formula A5, Formula A6, Formula A8, or Formula A10: 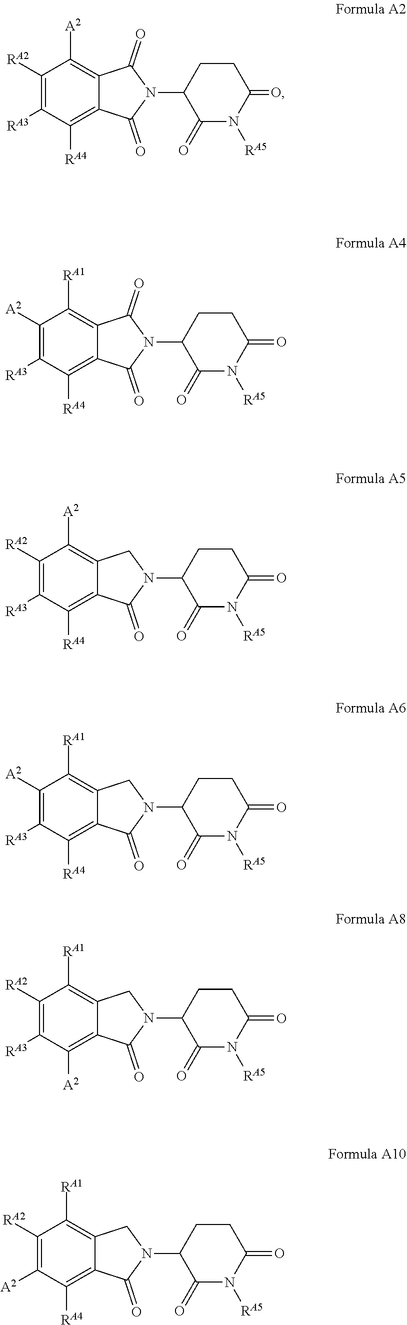
or a pharmaceutically acceptable salt thereof.

59.-63. (canceled)

64. The compound of claim 43, wherein the degradation moiety comprises the structure of

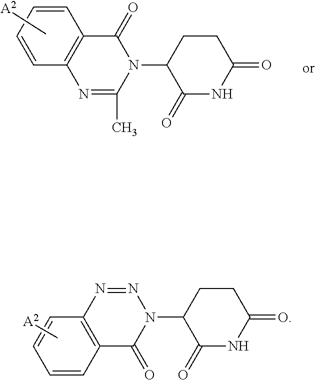

65. (canceled)

66. The compound of claim 41, or a pharmaceutically acceptable salt thereof, wherein the degradation moiety has the structure of Formula C: 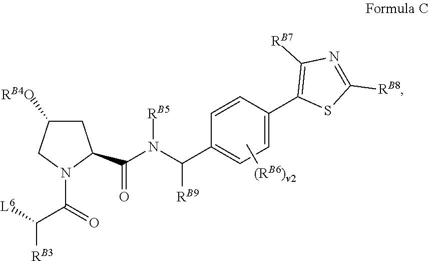
wherein
L⁶ is —N(R^{B1})(R^{B2}), 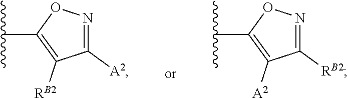
R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl; and
A² is a bond between the degradation moiety and the linker;
wherein one and only one of R^{B1}, R^{B3}, and R^{B6} is A².

67. The compound of claim 66, or a pharmaceutically acceptable salt thereof, wherein the degradation moiety has the structure of Formula C1 or Formula C2:

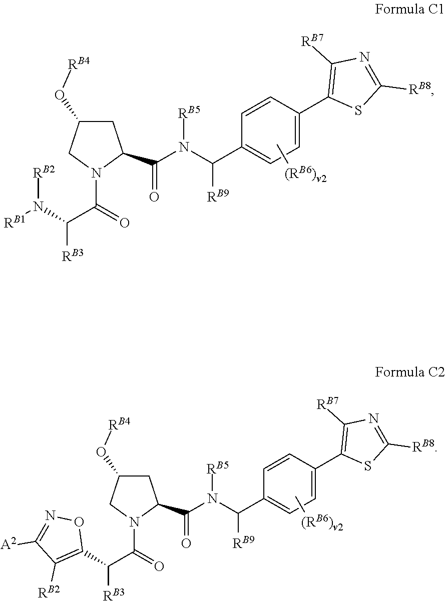

68. (canceled)

69. The compound of claim 66, or a pharmaceutically acceptable salt thereof, wherein R^{B9} is optionally substituted C₁-C₆ alkyl.

70. The compound of claim 69, or a pharmaceutically acceptable salt thereof, wherein R^{B9} is methyl.

71. The compound of claim 66, or a pharmaceutically acceptable salt thereof, wherein R^{B9} is bonded to (S)-stereogenic center.

72. The compound of claim 66, or a pharmaceutically acceptable salt thereof, wherein R^{B9} is hydrogen.

73. The compound of claim 66, or a pharmaceutically acceptable salt thereof, wherein the degradation moiety has the structure:

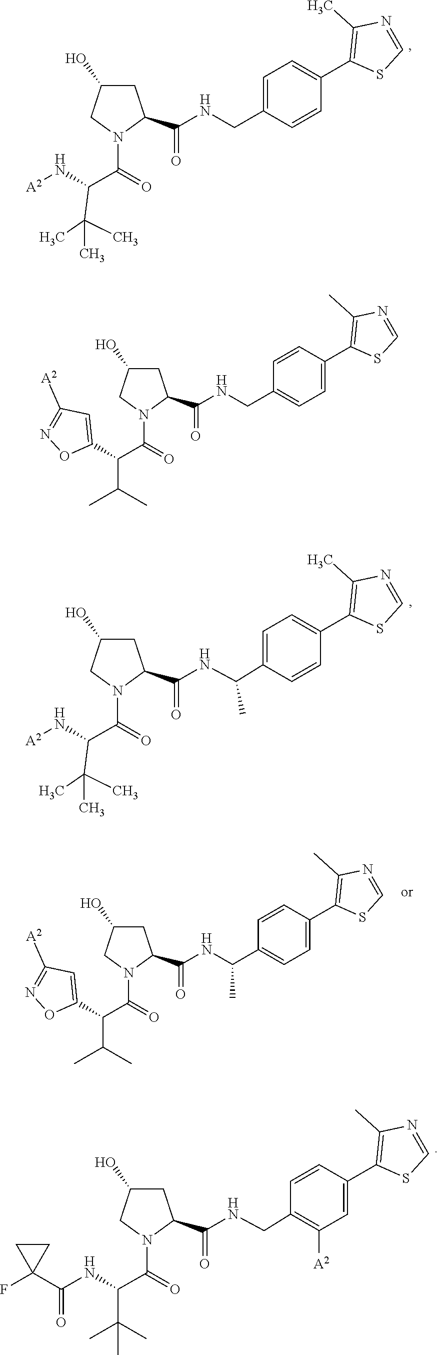

74.-77. (canceled)

78. The compound of claim 1, or a pharmaceutically acceptable salt thereof, wherein the degradation moiety comprises the structure of 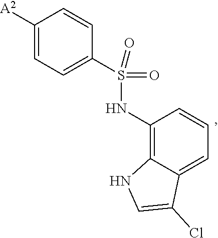
wherein A² is a bond between the degradation moiety and the linker.

79. A compound selected from the group consisting of compounds 1-75 in Table 1, and pharmaceutically acceptable salts thereof.

80. A compound selected from the group consisting of compounds 105-272 in Table 2, and pharmaceutically acceptable salts thereof.

81. The compound of claim 80, wherein the compound is any one of compounds 76-104 in Table 2, or a pharmaceutically acceptable salt thereof.

82. A pharmaceutical composition comprising a compound of claim 1 and a pharmaceutically acceptable excipient.

83. (canceled)

84. (canceled)

85. A method of treating a BAF complex-related disorder in a subject in need thereof, the method comprising administering to the subject an effective amount of a compound of claim 1.

86. The method of claim 85, wherein the BAF complex-related disorder is cancer or a viral infection.

87. (canceled)

88. (canceled)

89. A method of treating a disorder related to a BRG1 loss of function mutation in a subject in need thereof, the method comprising administering to the subject an effective amount of a compound of claim 1.

90. The method of claim 89, wherein the disorder related to a BRG1 loss of function mutation is cancer.

91. (canceled)

92. (canceled)

93. A method of treating cancer in a subject in need thereof, the method comprising administering to the subject an effective amount of a compound of claim 1.

94. The method of claim 93, wherein the cancer is non-small cell lung cancer, colorectal cancer, bladder cancer, cancer of unknown primary, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.

95. The method of claim 93, wherein the cancer is non-small cell lung cancer, colorectal cancer, bladder cancer, cancer of unknown primary, glioma, breast cancer, melanoma, non-melanoma skin cancer, endometrial cancer, or penile cancer.

96. (canceled)

97. (canceled)

98. A method of treating a cancer selected from the group consisting of melanoma, prostate cancer, breast cancer, bone cancer, renal cell carcinoma, and a hematologic cancer in a subject in need thereof, the method comprising administering to the subject an effective amount of a pharmaceutical composition of claim 82.

99. (canceled)

100. (canceled)

101. (canceled)

102. A method of reducing the level and/or activity of BRG1 and/or BRM in a cancer selected from the group consisting of melanoma, prostate cancer, breast cancer, bone cancer, renal cell carcinoma, and hematologic cancer cell, the method comprising contacting the cell with an effective amount a compound of claim 1.

103.-124. (canceled)

125. The method of claim 102, wherein the breast cancer is an ER positive breast cancer, an ER negative breast cancer, triple positive breast cancer, or triple negative breast cancer.

126.-131. (canceled)
